(19) United States
(12) Patent Application Publication
(10) Pub. No.: US 2024/0336909 A1
(43) Pub. Date: Oct. 10, 2024

(54) CONDITIONAL ACTIVATION OF IMMUNOGLOBULIN MOLECULES

(71) Applicant: Harpoon Therapeutics, Inc., South San Francisco, CA (US)

(72) Inventors: Shuoyen Jack LIN, San Bruno, CA (US); Richard J. AUSTIN, San Francisco, CA (US); Bryan D. LEMON, Mountain View, CA (US); Kathryn KWANT, San Bruno, CA (US); Sony ROCHA, San Francisco, CA (US); Holger WESCHE, San Francisco`, CA (US); Kevin WRIGHT, Belmont, CA (US)

(21) Appl. No.: 18/635,533

(22) Filed: Apr. 15, 2024

Related U.S. Application Data
(63) Continuation of application No. PCT/US2022/078178, filed on Oct. 14, 2022.
(60) Provisional application No. 63/256,122, filed on Oct. 15, 2021.

(51) Int. Cl. C12N 9/64; A61K 39/00; A61P 35/00; C07K 14/725; C07K 16/18

(52) U.S. Cl. CPC C12N 9/6491; A61P 35/00; C07K 14/7051; C07K 16/18; A61K 2039/505; C07K 2317/24; C07K 2317/565; C07K 2317/569

(57) ABSTRACT

Disclosed herein are binding moieties that comprise non-CDR loops for masking the binding of a binding molecule to its target and CDRs for binding bulk serum proteins. Conditionally active target binding proteins that contain the binding moieties are also provided. Cleavable linkers used in the conditionally active target binding proteins are also disclosed.

### CROSS-REFERENCE

This application is a continuation of International Application No. PCT/US2022/078178, filed Oct. 14, 2022, and claims the benefit of U.S. Provisional Application No. 63/256,122, filed Oct. 15, 2021, all of which are incorporated herein by reference in their entirety.

### SEQUENCE LISTING

The instant application contains a Sequence Listing which has been filed electronically in XML format and is hereby incorporated by reference in its entirety. Said XML copy, created on May 29, 2024, is named 47517_747_301.xml and is 1,264,298 bytes in size.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if set forth in their entireties.

### BACKGROUND OF THE DISCLOSURE

T cell engagers transiently tether T cells to tumor cells and mediate T cell-directed tumor killing. T cell engagers, such as blinatumomab (BLINCYTO®), have demonstrated clinical activity in several hematological malignancies. Adoption of T cell engagers in solid tumors is limited by the scarcity of tumor antigens with sufficient differential expression between tumor and normal tissue. T cell engagers that are preferentially active in the tumor microenvironment may enable the safe targeting of more solid tumor antigens.

There is a need to extend the half-life of a therapeutic, diagnostic, or imaging molecule in circulation and also improve its ability to reach its target within an intended location (e.g., a tumor cell) without non-specific binding.

### SUMMARY OF THE DISCLOSURE

Provided herein is a conditionally active binding protein comprising: a binding moiety (M) which comprises a non-CDR loop, a cleavable linker (L), and a first target antigen binding domain (T1), wherein the binding moiety is capable of masking the binding of the first target antigen binding domain (T1) to its target, wherein the cleavable linker comprises a protease cleavage site that is recognized by at least one protease selected from the group consisting of: Neutrophil elastase, MMP-12, and MMP-13, and wherein the protease cleavage site is not recognized by furin.

In some embodiments, the protease cleavage site is further recognized by at least one protease selected from the group consisting of: a matriptase and a Urokinase-type Plasminogen Activator (uPA). In some embodiments, the protease cleavage site is further recognized by a matriptase but not by a uPA. In some embodiments, the protease cleavage site is not recognized by at least one protease selected from the group consisting of: MMP-14, Cathepsin-G, a matriptase, and a uPA. In some embodiments, the protease cleavable site is not recognized by at least one protease selected from the group consisting of: a matriptase and a uPA. In some embodiments, the protease cleavage site is further recognized by at least one protease selected from the group consisting of: MMP-2, MMP-7, MMP-8, and MMP-9. In some embodiments, the protease cleavage site is further recognized by at least one protease selected from the group consisting of: MMP-1, MMP-2, MMP-8, and MMP-9. In some embodiments, the protease cleavage site is further recognized by at least one protease selected from the group consisting of: MMP-2 and MMP-9. In some embodiments, the protease cleavage site is further recognized by at least one protease selected from the group consisting of: MMP-2, MMP-7, and MMP-9. In some embodiments, the protease cleavage site is further recognized by at least one protease selected from the group consisting of: MMP-2, MMP-8, and MMP-9. In some embodiments, the protease cleavage site is further recognized by at least one protease selected from the group consisting of: MMP-2 and MMP-7. In some embodiments, the protease cleavage site recognition by a protease is assayed by determining a percent cleavage of the cleavable linker comprising the protease cleavage site, in an assay wherein the cleavable linker is incubated with the protease for a period for about 1 hour at a temperature of about 37° C.

Provided herein is a conditionally active binding protein comprising a binding moiety (M) which comprises a non-CDR loop, a cleavable linker (L), and a first target antigen binding domain (T1), wherein the binding moiety is capable of masking the binding of the first target antigen binding domain to its target, wherein the cleavable linker comprises an amino acid sequence selected from the group consisting of: SEQ ID NOs: 910-931 and 985-996, or an amino acid sequence comprising one or more mutations in a sequence selected from the group consisting of SEQ ID NOs: 910-931 and 985-996.

In some embodiments, the conditionally active binding protein further comprises a second target antigen binding domain (T2). In some embodiments, the first (T1) or the second (T2) target antigen binding domain independently binds to a tumor antigen. In some cases, the tumor antigen is selected from the group consisting of: EGFR, PSMA, EpCAM, BCMA, 5T4, AFP, Axl, B7-H3, Cadherin-6, CAIX, CD117, CD123, CD138, CD166, CD19, CD20, CD205, CD22, CD30, CD33, CD352, CD37, CD38, CD44, CD52, CD56, CD70, CD71, CD74, CD79b, CEACAM5, c-MET, DLL3, EphA2, FAP, FGFR2, FGFR3, glypican-3, FLT-3, FOLRI, gpNMB, HER2, HPV-16 E6, HPV-16 E7, ITGA3, SLC39A6, Mesothelin, Muc1, Muc16, NaPi2b, Nectin-4, P-cadherin, Prolactin R, PSCA, PTK7, ROR1, SLC44A4, SLTRK5, SLTRK6, STEAP1, TIM1, Trop2, and WT1. In some embodiments, the first (T1) or the second (T2) target antigen binding domain independently binds to an immune modulatory protein. In some cases, the immune modulatory protein is selected from the group consisting of: CTLA-4, CD27, CD137, 2B4, TIGIT, CD155, ICOS, HVEM, CD40L, LIGHT, TIM-1, OX40, DNAM-1, PD-L1, PD1, PD-L2, CD8, CD40, CEACAM1, CD48, CD70, A2AR, CD39, CD73, B7-H3, B7-H4, BTLA, IDO1, IDO2, TDO, KIR, LAG-3, TIM-3, VISTA, IL6-R, IL-6, TNFα, CD19, CD20, CD22, CD52, integrin a4, integrin a4b7, CD11a, CTLA4-Ig fusion, IL-17, IL12/23, IL12, IL23, and TGF-beta. In some cases, the first (T1) or the second (T2) target antigen binding domain independently binds to an immune cell. In some cases, the first (T1) or the second (T2) target antigen binding domain independently binds to a T-cell. In some cases, the first (T1) or the second (T2) target antigen binding domain independently binds to CD3. In some embodiments, the binding moiety (M), the cleavable linker (L), the first target antigen binding domain (T1), and the second target antigen binding domain (T2) are in one of the following configurations: M:L:T1:T2, M:L:T2:T1, T1:T2:L:M, and T2:T1:L:M.

In some embodiments, the binding moiety is capable of binding to a half-life extending protein. In some embodiments, the binding moiety is a natural peptide, a synthetic peptide, an engineered scaffold, or an engineered bulk serum protein. In some cases, the engineered scaffold comprises an sdAb, an scFv, a Fab, a VHH, a fibronectin type III domain, an immunoglobulin-like scaffold, a DARPin, a cystine knot peptide, a lipocalin, a three-helix bundle scaffold, a protein G-related albumin-binding module, a DNA aptamer scaffold, or an RNA aptamer scaffold. In some embodiments, the non-CDR loop is from a variable domain, a constant domain, a C1-set domain, a C2-set domain, an I-domain, or any combinations thereof. In some embodiments, the binding moiety further comprises complementarity determining regions (CDRs). In some embodiments, the binding moiety comprises a binding site specific for a bulk serum protein. In some cases, the bulk serum protein is selected from the group consisting of: albumin, transferrin, IgG1, IgG2, IgG4, IgG3, IgA monomer, Factor XIII, Fibrinogen, IgE, and pentameric IgM. In some embodiments, the binding moiety further comprises a binding site specific for an immunoglobulin light chain. In some cases, the immunoglobulin light chain is an Igκ free light chain. In some embodiments, the CDRs provide the binding site specific for the bulk serum protein or the immunoglobulin light chain, or any combinations thereof. In some embodiments, the binding moiety is capable of masking the binding of the first target antigen binding domain (T1) to its target via specific intermolecular interactions between the binding moiety and the first target antigen binding domain. In some embodiments, the non-CDR loop provides a binding site specific for binding of the binding moiety to the first target antigen binding domain.

In some embodiments, the conditionally active binding protein further comprises a half-life extension domain bound to the binding moiety, wherein the half-life extension domain provides the binding protein with a safety switch, and wherein upon cleavage of the linker the binding protein is activated by separation of the binding moiety and the half-life extension domain from the first target antigen binding domain, and the binding protein is thereby separated from the safety switch. In some cases, the cleavage of the cleavable linker is in a tumor microenvironment. In some embodiments, the non-CDR loop comprises a CC′ loop of at least one of: a camelid VHH domain, a human VH domain, a humanized VH domain, or a single domain antibody. In some embodiments, the binding moiety comprises a masking sequence, wherein the masking sequence comprises an amino acid sequence selected from the group consisting of SEQ ID NOs: 259-301 and 795, or a sequence comprising one or more mutations in a sequence selected from the group consisting of: SEQ ID NOs: 259-301 and 795. In some embodiments, the cleavable linker comprises a sequence selected from the group consisting of SEQ ID NOs: 910-931, or a sequence comprising one or more mutations in a sequence selected from the group consisting of SEQ ID NOs: 910-931. In some embodiments, the cleavable linker comprises a sequence selected from the group consisting of: SEQ ID NOs: 985-996, or a sequence comprising one or more mutations in a sequence selected from the group consisting of SEQ ID NOs: 985-996.

Provided herein is a method of treating a disease comprising administering to a subject an effective amount of a conditionally active binding protein. In some cases, the subject is a human. In some cases, the disease is a tumorous disease or an inflammatory disease. In some cases, the disease is the tumorous disease, and wherein the tumorous disease is characterized by overexpression of at least one of EpCAM, PSMA, EGFR, BCMA, DLL3, FLT3, or combinations thereof.

Provided herein is a cleavable linker comprising a sequence selected from the group consisting of: SEQ ID NOs: 915-917, 919, 920 and 929-931.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which.

FIG. 1 illustrates a variable domain of an exemplary immunoglobulin domain, comprising complementarity determining regions (CDR1, CDR2, and CDR3), and non-CDR loops connecting the beta strand (AB, CC′, C″ D, EF, and DE).

FIGS. 2A-2B provide exemplary arrangements of various domains of a conditionally active binding protein of this disclosure. FIG. 2A: Version 1. FIG. 2B: Version 2.

FIG. 3 shows an exemplary conditionally active target binding protein of this disclosure.

FIGS. 4A-4C show activation and possible mode of action of trispecific molecules (ProTriTAC). FIG. 4A shows ProTriTAC molecules in circulation, in tumor environment, and in circulation. FIG. 4B shows an exemplary sequence for the protease cleavable site in a linker tethered to an anti-albumin binding moiety and FIG. 4C shows an SDS-PAGE gel showing the ProTriTAC in its activatable (prodrug) and activated (active drug) states.

FIGS. 5A-B illustrate a process for making and purifying molecules described herein.

FIG. 5A shows a schematic flowchart for manufacturing a ProTriTAC molecule and FIG. 5B shows an SDS-PAGE gel showing three purified ProTriTAC molecules.

FIGS. 6A-B show analytical size exclusion chromatograms on a ProTriTAC molecule exposed to different stress conditions in graph form in FIG. 6A with the corresponding data in FIG. 6B. FIG. 6B provides the data for FIG. 6A.

FIG. 7 shows protease-dependent, anti-tumor activity of exemplary ProTriTAC molecules in HCT116 Colorectal Tumor Xenograft Model in NSG Mice.

FIGS. 8A-8D show various designs of exemplary ProTriTAC and control molecules.

FIG. 8A: Control #1; FIG. 8B: Control #2; FIG. 8C: ProTriTAC; and FIG. 8D: Activated ProTriTAC.

FIG. 9 shows pharmacokinetic profiles for exemplary ProTriTAC and control molecules.

FIG. 10 shows conversion and half-life of exemplary ProTriTAC molecule.

FIG. 11 shows plasma clearance of an exemplary ProTriTAC molecule and its converted active drug format.

FIG. 12 shows CD3 binding potential of an exemplary ProTriTAC molecule, its converted active drug format, and a control non-cleavable ProTriTAC molecule.

FIG. 13 shows human primary T cell binding potential of an exemplary ProTriTAC molecule, its converted active drug format, and a control non-cleavable ProTriTAC molecule.

FIG. 14 shows T cell killing potential of an exemplary ProTriTAC molecule, its converted active drug format, and a control non-cleavable ProTriTAC molecule.

FIG. 15 shows the schematic structure of an exemplary trispecific molecule containing a binding moiety as described herein (also referred to herein as ProTriTAC or activatable ProTriTAC).

FIGS. 16A-16E shows exemplary schematic structures for Prodrug molecules combining functional masking and half-life extension. FIG. 16A shows a ProDrug molecule comprising an anti-albumin moiety which includes a masking moiety, and a cleavable linker connecting the anti-albumin moiety and the drug. FIG. 16B shows a ProDrug molecule comprising an anti-albumin moiety comprising two peptide motifs linked by linker, one of which includes a masking moiety, and a cleavable linker connecting the albumin binding moiety to a drug. FIG. 16C shows a ProDrug molecule comprising a modified albumin (containing a masking moiety) linked to a drug by a cleavable linker. FIG. 16D shows a ProDrug molecule comprising a modified albumin (containing a masking moiety and a protease cleavage site) linked to a drug. FIG. 16E shows an activated ProDrug. In each schematic structure (FIGS. 16A-16D) the drug molecule is functionally masked by the anti-albumin moiety or the modified albumin from binding its target or from being activated at an undesired site or from binding at non-target sites and thereby creating a drug sink.

FIGS. 17A-17E show exemplary schematic structures for ProTriTAC molecules combining functional masking and half-life extension. FIG. 17A shows a ProTriTAC molecule comprising an anti-albumin moiety which includes a masking moiety, and a cleavable linker connecting the anti-albumin moiety and a T cell engager molecule. FIG. 17B shows a ProTriTAC molecule comprising an anti-albumin moiety comprising two peptide motifs linked by linker, one of which includes a masking moiety, and a cleavable linker connecting the albumin binding moiety to a T cell engager molecule. FIG. 17C shows a ProTriTAC molecule comprising a modified albumin (containing a masking moiety) linked to a T cell engager by a cleavable linker. FIG. 17D shows a ProTriTAC molecule comprising a modified albumin (containing a masking moiety and a protease cleavage site) linked to a T cell engager. FIG. 17E shows an activated ProTriTAC. In each schematic structure (FIGS. 17A-17D) a target binding interface within the ProTriTAC molecule is functionally masked by the anti-albumin moiety or the modified albumin from binding its target or from being activated at an undesired site or from binding at non-target sites and thereby creating a sink.

FIG. 18 shows anti-tumor activity of exemplary ProTriTAC molecules and TriTAC molecules of this disclosure.

FIG. 19 shows pharmacokinetic profile of exemplary ProTriTAC molecules and TriTAC molecules of this disclosure.

FIGS. 20A-20F show admix xenograft individual tumor volumes following administering exemplary ProTriTAC molecules or TriTAC molecules of this disclosure. FIG. 20A illustrates results from a GFP TriTAC. FIG. 20B. illustrates results from EGFR ProTriTAC (NCLV). FIG. 20C illustrates results from EGFR ProTriTAC (L001). FIG. 20D illustrates results from EGFR ProTriTAC (L041). FIG. 20E illustrates results from EGFR ProTriTAC (L040). FIG. 20F illustrates results from EGFR ProTriTAC (L045).

FIGS. 21A-21C shows cytokine levels (IFN-gamma (FIG. 21A), IL-6 (FIG. 21B), and IL-10; FIG. 21C) following administering exemplary ProTriTAC molecules or TriTAC molecules of this disclosure.

FIGS. 22A-22E show body weight percent change in mice, following administering exemplary ProTriTAC molecules and TriTAC molecules of this disclosure. FIG. 22A illustrates results of 30 μg/kg; FIG. 22B illustrates results of 100 μg/kg; FIG. 22C illustrates results of 300 μg/kg; FIG. 22D illustrates results of 1000 μg/kg; and FIG. 22E provides fold protection at the various concentrations.

FIGS. 23A-23C show body weight percent change in mice, following administering varying concentrations of exemplary ProTriTAC molecules of this disclosure, containing non-cleavable or cleavable linkers. FIG. 23A illustrates results of 300 μg/kg; FIG. 23B illustrates results of 1000 μg/kg; FIG. 23C provides fold protection at the various concentrations.

FIGS. 24A-24C show serum concentrations of aspartate aminotransferase (AST), in mice, following administering varying concentrations of a ProTriTAC molecule containing a non-cleavable linker (ProTriTAC (NCLV)) (FIG. 24C), a TriTAC molecule (FIG. 24A), or a ProtriTAC molecule (FIG. 24B) containing a cleavable linker.

FIGS. 25A-25C show serum concentrations of alanine aminotransferase (ALT), in mice, following administering varying concentrations of a ProTriTAC molecule containing a non-cleavable linker (ProTriTAC (NCLV)) (FIG. 25C), a TriTAC molecule (FIG. 25A), or a ProtriTAC molecule (FIG. 25B) containing a cleavable linker.

FIGS. 26A-26B show show serum concentrations of ALT (right panel; FIG. 26B) or AST (left panel; FIG. 26A), in cynomolgus monkeys, following administering varying concentrations of an EGFR ProTriTAC molecule, or an EGFR ProTriTAC (NCLV) molecule.

FIGS. 27A-27D show tumor volume in mice following administration of a GFP TriTAC molecule, an EGFR TriTAC molecule, or an EGFR ProTriTAC molecule, in varying concentrations. FIG. 27A shows GFP TriTAC (at 300 μg/kg) and an EGFR TriTAC (at 10 μg/kg). FIG. 27B shows the EGFR TriTAC (at 30 μg/kg and at 100 μg/kg). FIG. 27C shows the EGFR TriTAC (at 300 μg/kg) and an EGFR ProTriTAC (at 30 μg/kg and 100 μg/kg). FIG. 27D shows the EGFR ProTriTAC (at 300 μg/kg and 1000 μg/kg)

FIG. 28 shows serum concentrations of ALT and AST, in mice, following administration of varying concentrations of a GFP TriTAC, an EGFR TriTAC, and an EGFR ProTriTAC molecule.

FIG. 29 shows grafting of a CD36 epitope into the CC′ loop of a binding moiety of this disclosure. HuCD3e: SEQ ID NO: 901; CC10: SEQ ID NO: 260; CC12: SEQ ID NO: 259; and CC16: SEQ ID NO: 261.

FIG. 30 shows separation of a binding moiety of this disclosure, from a ProTriTAC molecule that contained the binding moiety, upon tumor associated protease activation by matriptase.

FIG. 31 shows CD3 binding of ProTriTAC molecules, with or without activation, containing an exemplary binding moiety of this disclosure.

FIG. 32 shows cell killing potential of a ProTriTAC molecule, with or without activation, containing an exemplary binding moiety of this disclosure.

FIG. 33 shows the soft library mutagenesis approach carried out to explore the non-CDR loops within an exemplary binding moiety of this disclosure.

FIG. 34 illustrates the results of the soft library mutagenesis approach carried out to explore the non-CDR loops within an exemplary binding moiety of this disclosure, after panning against HSA (human serum albumin or also referred to herein as albumin).

FIG. 35 illustrates that a binding moiety of this disclosure is able to expand the therapeutic window of a molecule containing it (e.g., a ProTriTAC molecule) by both steric masking and specific masking.

FIG. 36 provides results from a representative T cell dependency cellular cytotoxicity assay with NCI-H508 cells using exemplary fusion proteins of this disclosure containing an anti-EpCAM domain as described herein and an anti-CD3 domain.

FIG. 37 provides results from a representative T cell dependency cellular cytotoxicity assay using exemplary fusion proteins of this disclosure containing an anti-EpCAM domain as described herein and an anti-CD3 domain.

FIG. 38 provides results from a representative T cell dependency cellular cytotoxicity assay using exemplary fusion proteins of this disclosure containing an anti-EpCAM domain as described herein and an anti-CD3 domain.

FIG. 39 provides results from a representative T cell dependency cellular cytotoxicity assay using exemplary fusion proteins of this disclosure containing an anti-EpCAM domain as described herein and an anti-CD3 domain.

FIG. 40 provides results from a representative T cell dependency cellular cytotoxicity assay using exemplary fusion proteins of this disclosure containing a humanized anti-EpCAM domain as described herein and an anti-CD3 domain.

FIGS. 41A-41C show body weight percent change in mice, following administering exemplary EpCAM ProTriTAC molecules and EpCAM TriTAC molecules of this disclosure.

FIG. 42 shows body weight percent change in mice, following administering exemplary EGFR ProTriTAC molecules and EGFR TriTAC molecules of this disclosure. In particular, the linkers involved were NCLV (non-cleavable), L001, L040, L042, and L043.

FIGS. 43A-43C shows body weight percent change in mice, following administering exemplary EGFR ProTriTAC molecules and EGFR TriTAC molecules of this disclosure. FIG. 43A shows the results using linker sequences NCLV (non-cleavable) and L001 as controls, and linker sequences L054, L055, L059, L064, L065, L069. FIG. 43B shows the results using linker sequences NCLV and L001 as controls, and linker sequences L060, L061, L062, L063, L070, L071, L072, L073. FIG. 43C demonstrates the results conducted using NCLV, L001, and L040 as controls, and linker sequences L074 and L075.

FIG. 44 demonstrates ProTriTAChas substantially reduced pre-cleaved active drug in CHO productions with L276 versus L040 cleavable linker.

FIG. 45 provides protease substrates by sequence and selectivity to metalloproteinases.

FIGS. 46A-F illustrate exemplary ProCAR constructs. FIG. 46A illustrates construct C2483 which includes an anti-human serum albumin sdAb, a FLAG epitope, a CD8 hinge/transmembrane domain, a 4-1BB intracellular domain, and a CD3 zeta intracellular domain (SEQ ID NO: 1075). FIG. 46B illustrates construct C3544 which includes an anti-human serum albumin sdAb, an anti-human EpCAM sdAb, a FLAG epitope, a CD8 hinge/transmembrane domain, a 4-1BB intracellular domain, and a CD3 zeta intracellular domain (SEQ ID NO: 1076). FIG. 46C illustrates construct C3546 which includes an anti-human serum albumin sdAb that contains a mask in the CC′ loop, an anti-human EpCAM sdAb, a protease cleavable linker 4 (SEQ ID NO: 1077), a FLAG epitope, a CD8 hinge/transmembrane domain, a 4-1BB intracellular domain, and a CD3 zeta intracellular domain (SEQ ID NO: 1078).

FIG. 46D illustrates construct C3548 which includes an anti-human serum albumin sdAb that contains a mask in the CC′ loop, an anti-human EpCAM sdAb, a protease cleavable linker 5 (SEQ ID NO: 1079), a FLAG epitope, a CD8 hinge/transmembrane domain, a 4-1BB intracellular domain, and a CD3 zeta intracellular domain (SEQ ID NO: 1080). FIG. 46E illustrates construct C3549 which includes an anti-human serum albumin sdAb,an anti-human EpCAM sdAb, a protease cleavable linker 6 (SEQ ID NO: 1081), a FLAG epitope, a CD8 hinge/transmembrane domain, a 4-1BB intracellular domain, and a CD3 zeta intracellular domain (SEQ ID NO: 1082). FIG. 46F illustrates construct C3550 which includes an anti-human serum albumin sdAb that contains a mask in the CC′ loop, an anti-human EpCAM sdAb, a protease cleavable linker 7 (SEQ ID NO: 1083), a FLAG epitope, a CD8 hinge/transmembrane domain, a 4-1BB intracellular domain, and a CD3 zeta intracellular domain (SEQ ID NO: 1084).

FIGS. 47A-D illustrate that pan-MMP substrates are not stable when expressed on T cells in ProCAR format. CAR-T cells made from the constructs of FIG. 46 were prepared. Shown are dot plots of M2 staining (x-axis; BV421 channel) and EpCAM-Fc staining (y-axis; AlexaFluor 647 channel) of CAR-T cells. FIG. 47A provides results for constructs C2483, C3546, and C3544. FIG. 47B provides results for constructs C2483, C3548, and C3544. FIG. 47C provides results for constructs C2483, C3549, and C3544. FIG. 47D provides results for constructs C2483, C3550, and C3544.

FIG. 48 illustrates that MMP2 and MMP7 are expressed at very low levels in T cells compared to MMP9.

FIG. 49A-E illustrate exemplary ProCAR constructs. FIG. 49A illustrates construct C2483 which includes an anti-human serum albumin sdAb, a FLAG epitope, a CD8 hinge/transmembrane domain, a 4-1BB intracellular domain, and a CD3 zeta intracellular domain (SEQ ID NO: 1075). FIG. 49B illustrates construct C3269 includes an anti-human serum albumin sdAb, an anti-human EpCAM sdAb, a FLAG epitope, a CD8 hinge/transmembrane domain, a 4-1BB intracellular domain, and a CD3 zeta intracellular domain (SEQ ID NO: 1085). FIG. 49C illustrates construct C3440 which includes an anti-human serum albumin sdAb that contains a mask in the CC′ loop, an anti-human EpCAM sdAb, a protease cleavable linker 8 (SEQ ID NO: 1086), a FLAG epitope, a CD8 hinge/transmembrane domain, a 4-1BB intracellular domain, and a CD3 zeta intracellular domain (SEQ ID NO: 1087).

FIG. 49D illustrates construct C3444 which includes an anti-human serum albumin sdAb that contains a mask in the CC′ loop, an anti-human EpCAM sdAb, a protease cleavable linker 9 (SEQ ID NO: 1088), a FLAG epitope, a CD8 hinge/transmembrane domain, a 4-1BB intracellular domain, and a CD3 zeta intracellular domain (SEQ ID NO: 1102). FIG. 49E illustrates construct C3445 which includes an anti-human serum albumin sdAb, an anti-human EpCAM sdAb, a protease cleavable linker 10 (SEQ ID NO: 1089), a FLAG epitope, a CD8 hinge/transmembrane domain, a 4-1BB intracellular domain, and a CD3 zeta intracellular domain (SEQ ID NO: 1103).

FIG. 50 A-C illustrate that MMP2-selective substrates show increased stability when expressed on T cells in ProCAR format. CAR-T cells made from the constructs of FIG. 33 were prepared. Shown are dot plots of M2 staining (x-axis; BV421 channel) and EpCAM-Fc staining (y-axis; AlexaFluor 647 channel) of CAR-T cells. FIG. 50A provides results for constructs C3440 and C3269 for MMP2-selective substrate 1. FIG. 50B provides results for constructs C3444 and C3269 for MMP2-selective substrate 2. FIG. 50C provides results for constructs C3445 and C3269 for MMP2-selective substrate 3.

FIG. 51A-E illustrate exemplary ProCAR constructs. FIG. 51A illustrates construct C2483 which includes an anti-human serum albumin sdAb, a FLAG epitope, a CD8 hinge/transmembrane domain, a 4-1BB intracellular domain, and a CD3 zeta intracellular domain (SEQ ID NO: 1075). FIG. 51B illustrates construct C3543 includes an anti-human serum albumin sdAb, an anti-human EpCAM sdAb, a FLAG epitope, a CD8 hinge/transmembrane domain, a 4-1BB intracellular domain, and a CD3 zeta intracellular domain (SEQ ID NO: 1090). FIG. 51C illustrates construct C3558 which includes an anti-human serum albumin sdAb that contains a mask in the CC′ loop, an anti-human EpCAM sdAb, a protease cleavable linker 11 (SEQ ID NO: 1091), a FLAG epitope, a CD8 hinge/transmembrane domain, a 4-1BB intracellular domain, and a CD3 zeta intracellular domain (SEQ ID NO: 1092). FIG. 51D illustrates construct C3559 which includes an anti-human serum albumin sdAb that contains a mask in the CC′ loop, an anti-human EpCAM sdAb, a protease cleavable linker 12 (SEQ ID NO: 1093), a FLAG epitope, a CD8 hinge/transmembrane domain, a 4-1BB intracellular domain, and a CD3 zeta intracellular domain (SEQ ID NO: 1094). FIG. 51E illustrates construct C3560 which includes an anti-human serum albumin sdAb, an anti-human EpCAM sdAb, a protease cleavable linker 13 (SEQ ID NO: 1095), a FLAG epitope, a CD8 hinge/transmembrane domain, a 4-1BB intracellular domain, and a CD3 zeta intracellular domain (SEQ ID NO: 1096).

FIGS. 52A-C illustrate that MMP-7-selective substrates show increased stability when expressed on T cells in ProCAR format. CAR-T cells made from the constructs of FIG. 51 were prepared. Shown are dot plots of M2 staining (x-axis; BV421 channel) and EpCAM-Fc staining (y-axis; AlexaFluor 647 channel) of CAR-T cells. FIG. 52A provides results for constructs C2483, C3558, and C3543 for MMP7-selective substrate 1. FIG. 52B provides results for constructs C2483, C3559, and C3543 for MMP7-selective substrate 2. FIG. 52C provides results for constructs C2483, C3560, and C3543 for MMP7-selective substrate 3.

FIG. 53A-C illustrate that MMP7-selective substrates are cleavable with recombinant MMP7. CAR-T cells made from the constructs of FIG. 51 were prepared. Shown are dot plots of M2 staining (x-axis; BV421 channel) and EpCAM-Fc staining (y-axis; AlexaFluor 647 channel) of CAR-T cells. FIG. 53A provides results for constructs C3558 and C3558+MMP7 for MMP7-selective substrate 1. FIG. 53B provides results for constructs C3559 and C3559+MMP7 for MMP7-selective substrate 2. FIG. 53C provides results for constructs C3560 and C3560+MMP7 for MMP7-selective substrate 3.

FIG. 54A-E illustrate exemplary ProCAR constructs. FIG. 54A illustrates construct C2446 which includes an anti-human serine albumin sdAb, a protease cleavage site 3, an anti-human EGFR sdAb, a FLAG epitope, a CD8 hinge/transmembrane domain, a 4-1BB intracellular domain, and a CD3 zeta intracellular domain (SEQ ID NO: 1097). FIG. 54B illustrates construct C2510 which includes an anti-human serine albumin sdAb, a protease cleavage site, an anti-human EGFR sdAb, a FLAG epitope, a dimerization-deficient CD8a hinge/transmembrane domain, a 4-1BB intracellular domain, and a CD3 zeta intracellular domain (SEQ ID NO: 1098). FIG. 54C illustrates construct C2513 which includes an anti-human serine albumin sdAb, a protease cleavage site 3, an anti-human EGFR sdAb, a FLAG epitope, a dimerization-deficient CD8a hinge/transmembrane domain, a 4-1BB intracellular domain, and a CD3 zeta intracellular domain (SEQ ID NO: 1099). FIG. 54D illustrates construct C2514 which includes an anti-human serine albumin sdAb, an anti-human EGFR sdAb, a FLAG epitope, a dimerization-deficient CD8a hinge/transmembrane domain, a 4-1BB intracellular domain, and a CD3 zeta intracellular domain (SEQ ID NO: 1100). FIG. 54E illustrates construct C2448, which includes an anti-human EGFR sdAb, a FLAG epitope, a dimerization-deficient CD8a hinge/transmembrane domain, a 4-1BB intracellular domain, and a CD3 zeta intracellular domain (SEQ ID NO: 1101).

FIG. 55A-B illustrate that the level of protease site-dependent cell killing activity of the ProCARs constructs. The level of protease site-dependent cell killing activity of the ProCARs constructs was reduced by eliminating the dimerization activity of the CD8a transmembrane domain (FIG. 55B) compared to wild-type (FIG. 55A)

FIG. 56 illustrates that the dimerization deficient EGFR CAR ad similar cell killing activity compared to wild-type.

FIG. 57 shows all Epcam targeting ProTriTAC molecules comprising a cleavable linker were able to reduce tumor growth at both 0.1 mg./kg and 0.3 mg/kg dose.

### DETAILED DESCRIPTION OF THE DISCLOSURE

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Provided herein in certain embodiments are ProTriTAC molecules (also referred to herein as protrisecific molecules) that are T cell engager prodrugs designed to be conditionally active in a tumor microenvironment. In some cases, this enables targeting of a wider selection of tumor antigens (e.g., solid tumor antigens). The ProTriTAC molecules, in some examples, combine the desirable attributes of several prodrug approaches, including, but not limited to: combination of steric and specific masking, wherein the steric masking is, in some cases, is through albumin that is recognized by an anti-albumin domain in a ProTriTAC molecule, and the specific masking, in some cases, is through specific intermolecular interactions between an anti-albumin domain (in some examples) and a target antigen binding domain of the ProTriTAC molecule (such as, an anti-CD3 scFv domain, in some examples); additional safety imparted by half-life differential of prodrug versus an active drug, derived by activation of the conditionally activated ProTriTAC molecule; ability to plug-and-play with different tumor target binders.

### Certain Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

The terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker).

A “single chain Fv” or “scFv”, as used herein, refers to a binding protein in which the variable domains of the heavy chain and of the light chain of a traditional two chain antibody are joined to form one chain. Typically, a linker peptide is inserted between the two chains to allow for proper folding and creation of an active binding site.

A “cleavage site for a protease,” or “protease cleavage site”, as meant herein, is an amino acid sequence that can be cleaved by a protease, such as, for example, a matrix metalloproteinase or a furin. Examples of such sites include Gly-Pro-Leu-Gly-Ile-Ala-Gly-Gln (SEQ ID NO: 1120) or Ala-Val-Arg-Trp-Leu-Leu-Thr-Ala (SEQ ID NO: 1121), which can be cleaved by metalloproteinases, and Arg-Arg-Arg-Arg-Arg-Arg, which is cleaved by a furin. In therapeutic applications, the protease cleavage site can be cleaved by a protease that is produced by target cells, for example cancer cells or infected cells, or pathogens.

As used herein, “elimination half-time” is used in its ordinary sense, as is described in Goodman and Gillman's The Pharmaceutical Basis of Therapeutics 21-25 (Alfred Goodman Gilman, Louis S. Goodman, and Alfred Gilman, eds., 6th ed. 1980). Briefly, the term is meant to encompass a quantitative measure of the time course of drug elimination. The elimination of most drugs is exponential (i.e., follows first-order kinetics), since drug concentrations usually do not approach those required for saturation of the elimination process. The rate of an exponential process may be expressed by its rate constant, k, which expresses the fractional change per unit of time, or by its half-time, t_{1/2} the time required for 50% completion of the process. The units of these two constants are time⁻¹ and time, respectively. A first-order rate constant and the half-time of the reaction are simply related (k×t_{1/2}=0.693) and may be interchanged accordingly. Since first-order elimination kinetics dictates that a constant fraction of drug is lost per unit time, a plot of the log of drug concentration versus time is linear at all times following the initial distribution phase (i.e., after drug absorption and distribution are complete). The half-time for drug elimination can be accurately determined from such a graph.

A “therapeutic agent,” as used herein, includes a “binding molecule.”

The term “binding molecule,” as used herein is any molecule, or portion or fragment thereof, that can bind to a target molecule, cell, complex and/or tissue, and which includes proteins, nucleic acids, carbohydrates, lipids, low molecular weight compounds, and fragments thereof, each having the ability to bind to one or more of a soluble protein, a cell surface protein, a cell surface receptor protein, an intracellular protein, a carbohydrate, a nucleic acid, a hormone, or a low molecular weight compound (small molecule drug), or a fragment thereof. The binding molecule, in some instances, is a protein belonging to the immunoglobulin superfamily, or a non-immunoglobulin binding molecule. The “binding molecule” does do not include a cytokine.

The term “proteins belonging to immunoglobulin superfamily,” or “immunoglobulin molecules,” as used herein, include proteins that comprise an immunoglobulin fold, such as antibodies and target antigen binding fragments thereof, antigen receptors, antigen presenting molecules, receptors on natural killer cells, antigen receptor accessory molecules, receptors on leukocytes, IgSF cellular adhesion molecules, growth factor receptors, and receptor tyrosine kinases/phosphatases.

The term “antibodies” include antibodies or immunoglobulins of any isotype, fragments of antibodies that retain specific binding to antigen, including, but not limited to, Fab, Fv, scFv, and Fd fragments, chimeric antibodies, humanized antibodies, single-chain antibodies (scAb), single domain antibodies (dAb), single domain heavy chain antibodies, a single domain light chain antibodies, bi-specific antibodies, multi-specific antibodies, and fusion proteins comprising an antigen-binding (also referred to herein as antigen binding) portion of an antibody and a non-antibody protein. The antibodies, in some examples, are detectably labeled, e.g., with a radioisotope, an enzyme that generates a detectable product, a fluorescent protein, and the like. The antibodies, in some cases, are further conjugated to other moieties, such as members of specific binding pairs, e.g., biotin (member of biotin-avidin specific binding pair), and the like. The antibodies, in some cases, are bound to a solid support, including, but not limited to, polystyrene plates or beads, and the like. Also encompassed by the term are Fab′, Fv, F(ab′)2, and or other antigen binding fragments that retain specific binding to antigen, and monoclonal antibodies. As used herein, a monoclonal antibody is an antibody produced by a group of identical cells, all of which were produced from a single cell by repetitive cellular replication. That is, the clone of cells only produces a single antibody species. While a monoclonal antibody can be produced using hybridoma production technology, other production methods known to those skilled in the art can also be used (e.g., antibodies derived from antibody phage display libraries). An antibody, in some instances, is monovalent or bivalent. An antibody, in some instances, is an Ig monomer, which is a “Y-shaped” molecule that consists of four polypeptide chains: two heavy chains and two light chains connected by disulfide bonds.

The term “non-immunoglobulin binding molecules,” as used herein, include, but is not limited to examples such as a growth factor, a hormone, a signaling protein, an inflammatory mediator, ligand, a receptor, or a fragment thereof, a native hormone or a variant thereof being able to bind to its natural receptor; a nucleic acid or polynucleotide sequence being able to bind to complementary sequence or a soluble cell surface or intracellular nucleic acid/polynucleotide binding proteins, a carbohydrate binding moiety being able to bind to other carbohydrate binding moieties, cell surface or intracellular proteins, a low molecular weight compound (drug) that binds to a soluble or cell surface or intracellular target protein. The non-immunoglobulin binding molecules, in some cases, include coagulation factors, plasma proteins, fusion proteins, and imaging agents. The non-immunoglobulin binding molecules do not include a cytokine.

A “cytokine,” as meant herein, refers to intercellular signaling molecules, and active fragments and portions thereof, which are involved in the regulation of mammalian somatic cells. A number of families of cytokines, for example, interleukins, interferons, and transforming growth factors are included.

As used herein, “non-CDR loops” within immunoglobulin (Ig) molecules are regions of a polypeptide other than the complementarity determining regions (CDRs) of an antibody. These regions may be derived from an antibody or an antibody fragment. These regions may also be synthetically or artificially derived, such as through mutagenesis or polypeptide synthesis.

In an Ig, Ig-like, or beta-sandwich scaffold that has 9 beta-strands (e.g., a VH, a VL, a camelid VHH, a sdAb), the non-CDR loops can refer to the AB, CC′, C″D, EF loops or loops connecting beta-strands proximal to the C-terminus. In an Ig, Ig-like, or beta-sandwich scaffold that has 7 beta-strands (e.g., a CH, a CL, an adnectin, a Fn-III), the non-CDR loops can refer to the AB, CD, and EF loops or loops connecting beta-strands proximal the C-terminus. In other Ig-like or beta-sandwich scaffolds, the non-CDR loops are the loops connecting beta-strands proximal to the C-terminus or topologically equivalent residues using the framework established in the Halaby 1999 publication (Prot Eng Des Sel 12:563-571).

In a non-beta-sandwich scaffold (e.g., a DARPin, an affimer, an affibody), the “non-CDR loops” refer to an area that is (1) amenable for sequence randomization to allow engineered specificities to a second antigen, and (2) distal to the primary specificity determining region(s) typically used on the scaffold to allow simultaneous engagement of the scaffold to both antigens without steric interference. For this purpose, the primary specificity determining region(s) can be defined using the framework established in the Skrlec 2015 publication (Trends in Biotechnol, 33:408-418). An excerpt of the framework is listed below.

| Scaffold | Primary specificity determining region(s) |
|---|---|
| Affibody | 13 residues in two helices |
| Affimer | 12-36 residues |
| Anticalin | Four loops (up to 24 aa) |
| Avimer | 11 residues |
| Centyrin | 13 residues |
| DARPin | 7 residues in each n-repeat, or 8 residues in each n-repeat |
| Fynomer | 6 residues in the RT- and n-Src-loop |
| Kunitz domain | 1-2 loops |

“Target antigen binding domain”, as used herein, refers to a region which targets a specific antigen. A target antigen binding domain comprises, for example an sdAb, an scFv, a variable heavy chain antibody (VHH), a variable heavy (VH) or a variable light domain (VL), a full-length antibody, or any other peptide that has a binding affinity towards a specific antigen. The target antigen binding domain does do not include a cytokine.

“TriTAC,” as used herein refers to a trispecific binding protein that is not conditionally activated.

### Binding Moiety, Cleavable Linker and Conditionally Active Binding Proteins

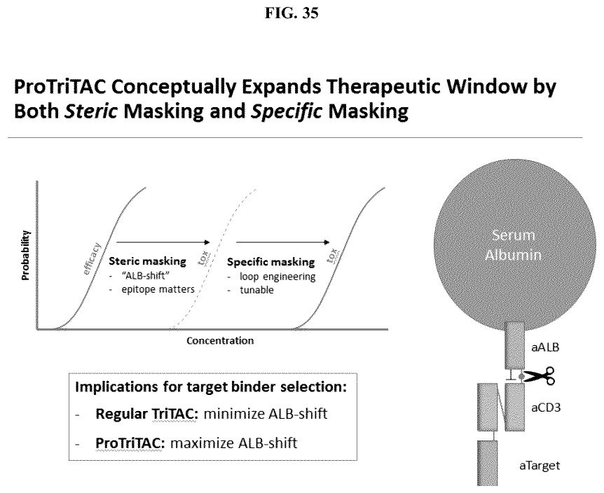

This disclosure provides, in some embodiments, binding moieties that are capable of masking the interaction of binding molecules with their targets. In some embodiments, a binding moiety of this disclosure comprises a masking moiety and a cleavable linker, such as a protease cleavable linker. In some embodiments, a binding moiety of this disclosure comprises a masking moiety (e.g., a modified non-CDR loop sequence) and a non-cleavable linker. As illustrated in FIG. 35, the binding moiety is capable of synergistically expanding a therapeutic window of a molecule that comprises the moiety, by both steric masking and specific masking. In some examples, the binding molecule is a protein belonging to an immunoglobulin superfamily, such as a target antigen binding domain comprising an immunoglobulin fold. In some embodiments, the binding molecule is a non-immunoglobulin protein. In some embodiments, the binding moiety combines both steric masking (for example, via binding to a bulky serum albumin) and specific masking (for example, via non-CDR loops binding to the CDRs of an anti-CD3 scFv domain). In some cases, modifying the non-CDR loops within the binding moiety does not affect albumin binding. The protease cleavable linker, in some cases, enables activation of a prodrug molecule comprising the binding moiety (such as a ProTriTAC molecule comprising a binding moiety as described herein, a CD3 binding domain, and an albumin binding domain), in a single proteolytic event, thereby allowing more efficient conversion of the prodrug molecule in tumor microenvironment. Further, tumor-associated proteolytic activation, in some cases, reveals active T cell engager (such as a ProTriTAC molecule comprising a binding moiety as described herein, a CD3 binding domain, and an albumin binding domain) with minimal off-tumor activity after activation. The present disclosure, in some embodiments, provides a half-life extended T cell engager format (ProTriTAC) comprising a binding moiety as described herein, which in some cases represents a new and improved approach to engineer conditionally active T cell engagers.

In some embodiments, the ProTriTAC molecule is protease-activated. In some embodiments, the protease-activated ProTriTAC molecule is about 2 times, 5 times, 10 times, 20 times, 30 times, 40 times, 50 times, 60 times, 70 times, 80 times, 90 times, 100 times, 120 times, 140 times, 150 times, 160 times, 180 times, 200 times, 220 times, 250 times, 270 times, 300 times, 325 times, 350 times, 375 times, 400 times, 425 times, 450 times, 475 times, or 500 times more potent in binding CD3 as compared to a non-cleavable ProTriTAC molecule in CD3 binding and T cell mediated cell killing. In some embodiments, the protease-activated ProTriTAC molecule is about 2 times, 5 times, 10 times, 20 times, 30 times, 40 times, 50 times, 60 times, 70 times, 80 times, 90 times, 100 times, 120 times, 140 times, 150 times, 160 times, 180 times, 200 times, 220 times, 250 times, 270 times, 300 times, 325 times, 350 times, 375 times, 400 times, 425 times, 450 times, 475 times, or 500 times more potent in binding CD3 as compared to a recombinant active drug fragment mimicking the protease-activated ProTriTAC molecule, in CD3 binding and T cell mediated cell killing. In some embodiments, the ProTriTAC molecules target EGFR. In some embodiments, an EGFR targeting protease-activated cleavable ProTriTAC molecule is about 1.1 times, 1.2 times, 1.3. times, 1.5 times, 2 times, 3 times, 4 times, 5 times, or about 10 times more potent in arresting tumor growth compared to an EGFR targeting non-cleavable ProTriTAC molecule. IN some embodiments, the protease-activated cleavable ProTriTAC molecule has about 2 fold, 5 fold, 10 fold, 20 fold, 30 fold, 40 fold, 50 fold, 60 fold, 70 fold, 80 fold, 90 fold, 100 fold, 120 fold, 140 fold, 160 fold, 180 fold, 200 fold or 250 fold longer circulating exposure compared to a non-cleavable ProTriTAC molecule.

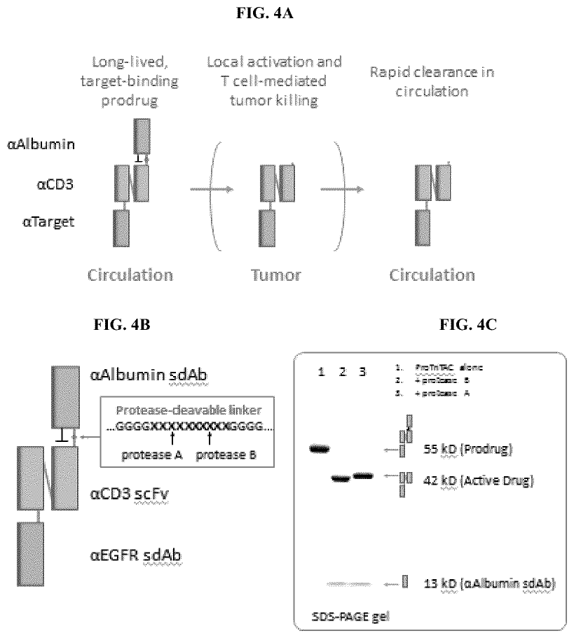

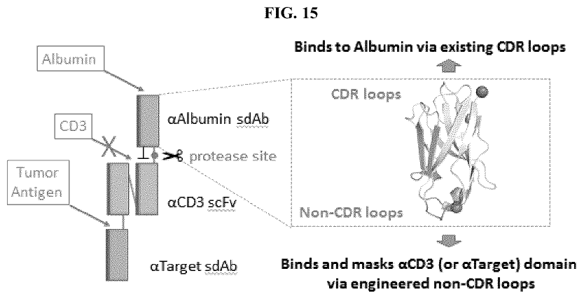

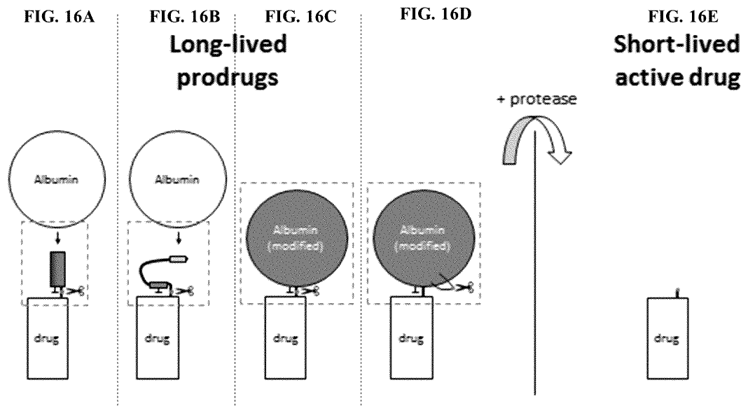

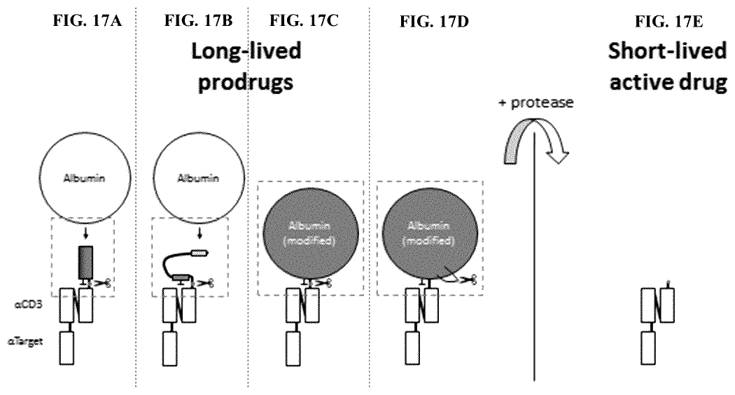

FIG. 4B provides a schematic for an exemplary ProTriTAC molecule comprising an exemplary binding moiety as described herein (the a albumin sdAb) and a gel showing the ProTriTAC before and after activation by cleaving of the protease cleavable linker, and FIG. 4A shows a possible mode of action of the same. FIG. 15 shows the schematic structure of an exemplary trispecific molecule containing a binding moiety as described herein (also referred to herein as ProTriTAC or activatable ProTriTAC), with engineered non-CDR loops. The exemplary trispecific molecule contains an anti-albumin domain comprising a cleavable linker (such a linker comprising a protease cleavable site, also referred to herein as a substrate linker) and a masking domain; an anti-CD3 binding domain; and an anti-target domain (specific for a tumor antigen) which in some cases is a non-immunoglobulin molecule. In some cases, non-CDR loops in the anti-albumin domain is capable of binding and masking the anti-target domain. In some cases, non-CDR loops in the anti-albumin domain is capable of binding and masking the anti-CD3 domain. The binding moiety, in some embodiments, comprises a CDR loop specific for binding albumin.

Provided herein, in a first embodiment, is a binding moiety that masks the binding of a target antigen binding domain and is capable of binding to a bulk-serum protein, such as a half-life extending protein. The binding moiety of the first embodiment, in certain instances, further comprises a cleavable linker attached to it. The cleavable linker, for example, comprises a protease cleavage site or a pH dependent cleavage site. The cleavable linker, in certain instances, is cleaved only in a tumor micro-environment. Thus, in some examples, the binding moiety of the first embodiment, bound to the half-life extending protein, connected to the cleavable linker, and further bound to the target antigen binding domain, maintains the target antigen binding domain in an inert state in circulation until the cleavable linker is cleaved off in a tumor microenvironment. The half-life of the target antigen binding domain, such as an antibody or an antigen binding fragment thereof, is thus extended in systemic circulation by using the binding moiety of the first embodiment which acts as a safety switch that keeps the target antigen binding moiety in an inert state until it reaches the tumor microenvironment where it is conditionally activated by cleavage of the linker and is able to bind its target antigen. In some embodiments, a protease cleavage site comprises an amino acid sequence selected from a group consisting of SEQ ID NOs: 1108-1117, 1120, and/or 1121.

In a second embodiment is provided a binding moiety that masks the interaction between a non-immunoglobulin binding molecule and its target. The binding moiety of the second embodiment, in certain instances, is capable of binding to a bulk serum protein. In some instances, the binding moiety of the second embodiment further comprises a cleavable linker attached to it. The cleavable linker, for example, comprises a protease cleavage site or a pH dependent cleavage site. In some embodiments, the cleavable linker comprises a protease cleavage site comprising an amino acid sequence selected from the group consisting of SEQ ID NOs: 1108-1117, 1120, and/or 1121. The cleavable linker, in certain instances, is cleaved only in a tumor micro-environment. The non-immunoglobulin binding molecule is, in some cases, maintained in an inert state by the binding moiety of the second embodiment and activated by cleavage of the linker, for example in a target environment. In some instances, the cleavable linker is cleaved off in a tumor microenvironment and in such cases the tumor microenvironment is the target environment. The half-life of the non-immunoglobulin binding molecule is thus extended in systemic circulation by using the binding moiety of the second embodiment which acts as a safety switch that keeps the non-immunoglobulin binding molecule in an inert state until it reaches the target environment where it is conditionally activated by cleavage of the linker. In some examples of the second embodiment where the non-immunoglobulin binding molecule is an imaging agent, said agent is activated in a target environment upon cleavage of the cleavable linker. The target environment, in such cases, is a tissue or a cell or any biological environment that is to be imaged using the imaging agent.

The safety switch described above provides several advantages: some examples including (i) expanding the therapeutic window of the immunoglobulin molecule, such as a target antigen binding domain, a non-immunoglobulin binding molecule; (ii) reducing target-mediated drug disposition by maintaining the immunoglobulin molecule, such as a target antigen binding domain, the non-immunoglobulin binding molecule, in an inert state when a conditionally active protein comprising a binding moiety according to the first or second embodiments is in systemic circulation; (iii) reducing the concentration of undesirable activated proteins in systemic circulation, thereby minimizing the spread of chemistry, manufacturing, and controls related impurities, e.g., pre-activated drug product, endogenous viruses, host-cell proteins, DNA, leachables, anti-foam, antibiotics, toxins, solvents, heavy metals; (iv) reducing the concentration of undesirable activated proteins in systemic circulation, thereby minimizing the spread of product related impurities, aggregates, breakdown products, product variants due to: oxidation, deamidation, denaturation, loss of C-term Lys in MAbs; (v) preventing aberrant activation of the immunoglobulin molecule, such as a target antigen binding domain, or the non-immunoglobulin binding molecule in circulation; (vi) reducing the toxicities associated with the leakage of activated species from diseased tissue or other pathophysiological conditions, e.g., tumors, autoimmune diseases, inflammations, viral infections, tissue remodeling events (such as myocardial infarction, skin wound healing), or external injury (such as X-ray, CT scan, UV exposure); and (vii) reducing non-specific binding of the immunoglobulin molecule, such as a target antigen binding domain, or the non-immunoglobulin binding molecule. Furthermore, post-activation, or in other words post breaking of the safety switch, the immunoglobulin molecule, such as a target antigen binding domain, the non-immunoglobulin binding molecule is separated from the safety switch which provided extended half-life, and thus is cleared from circulation.

In addition, the binding moieties of the first, second, and the third embodiments, in some cases, are used to generate a “biobetter” version of a biologic. Generally, preparing a biobetter form of a molecule, e.g., an antibody or an antigen binding fragment thereof, involves taking the originator molecule and making specific alterations in it to improve its parameters and thereby make it a more efficacious, less frequently dosed, better targeted, and/or a better tolerated drug. Thus, a target antigen binding domain masked by the binding moiety of the first embodiment which is bound to a half-life extending protein, and conditionally activated in a tumor microenvironment by cleavage of the cleavable linker, gives the target antigen binding domain a significantly longer serum half-life and reduces the likelihood of its undesirable activation in circulation, thereby producing a “biobetter” version of the target antigen binding domain. Similarly, the binding moieties of the second embodiment are, in some cases, utilized to generate biobetter versions of the non-immunoglobulin binding molecules. Accordingly, in various embodiments, biobetter versions of immunoglobulin molecules, non-immunoglobulin binding molecules are provided, wherein the biobetter function is attributed to a binding moiety, respectively, according to the first or second embodiments.

The binding moieties described herein comprise at least one non-CDR loop. In some embodiments, a non-CDR loop provides a binding site for binding of the binding moiety of the first embodiment to a target antigen binding domain. In some examples of the first embodiment, a non-CDR loop provides a binding site for binding of the binding moiety of the first embodiment to an immunoglobulin molecule, such as a target antigen binding domain. In some examples of the second embodiment, a non-CDR loop provides a binding site for binding of the binding moiety of the second embodiment to a non-immunoglobulin binding molecule. In some cases, the binding moiety of the first embodiment masks binding of the target binding domain to the target antigen, e.g., via steric occlusion, via specific intermolecular interactions, or a combination of both. The binding moieties of the second embodiment also, in some cases, mask binding of a non-immunoglobulin binding molecule to their targets, via steric occlusion, via specific intermolecular interactions, or a combination of both.

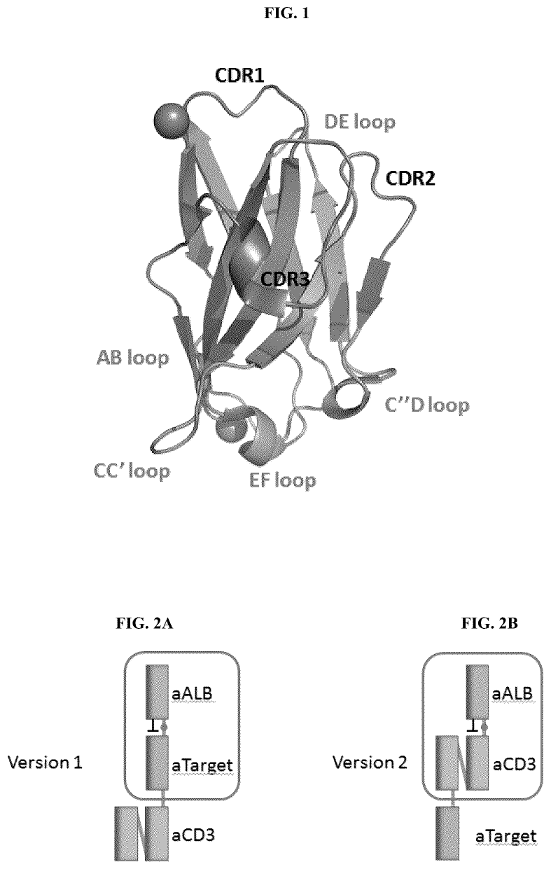

In some embodiments, the binding moieties described herein further comprise complementarity determining regions (CDRs). In some instances, the binding moieties are domains derived from an immunoglobulin molecule (Ig molecule). The Ig may be of any class or subclass (IgG1, IgG2, IgG3, IgG4, IgA, IgE, IgM etc). A polypeptide chain of an Ig molecule folds into a series of parallel beta strands linked by loops. In the variable region, three of the loops constitute the “complementarity determining regions” (CDRs) which determine the antigen binding specificity of the molecule. An IgG molecule comprises at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDRs) with are hypervariable in sequence and/or involved in antigen recognition and/or usually form structurally defined loops, interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In some embodiments of this disclosure, at least some or all of the amino acid sequences of FR1, FR2, FR3, and FR4 are part of the “non-CDR loop” of the binding moieties described herein. As shown in FIG. 1, a variable domain of an immunoglobulin molecule has several beta strands that are arranged in two sheets. The variable domains of both light and heavy immunoglobulin chains contain three hypervariable loops, or complementarity-determining regions (CDRs). The three CDRs of a V domain (CDR1, CDR2, CDR3) cluster at one end of the beta barrel. The CDRs are the loops that connect beta strands B-C, C′-C″, and F-G of the immunoglobulin fold, whereas the bottom loops that connect beta strands AB, CC′, C″-D and E-F of the immunoglobulin fold, and the top loop that connects the D-E strands of the immunoglobulin fold are the non-CDR loops. In some embodiments of this disclosure, at least some amino acid residues of a constant domain, CH1, CH2, or CH3, are part of the “non-CDR loop” of the binding moieties described herein. Non-CDR loops comprise, in some embodiments, one or more of AB, CD, EF, and DE loops of a C1-set domain of an Ig or an Ig-like molecule; AB, CC′, EF, FG, BC, and EC′ loops of a C2-set domain of an Ig or an Ig-like molecule; DE, BD, GF, A(A1A2)B, and EF loops of I(Intermediate)-set domain of an Ig or Ig-like molecule.

Within the variable domain, the CDRs are believed to be responsible for antigen recognition and binding, while the FR residues are considered a scaffold for the CDRs. However, in certain cases, some of the FR residues play an important role in antigen recognition and binding. Framework region residues that affect Ag binding are divided into two categories. The first are FR residues that contact the antigen, thus are part of the binding-site, and some of these residues are close in sequence to the CDRs. Other residues are those that are far from the CDRs in sequence but are in close proximity to it in the 3-D structure of the molecule, e.g., a loop in heavy chain.

In some embodiments, the non-CDR loop is modified to generate an antigen binding site specific for a bulk serum protein, such as albumin. It is contemplated that various techniques can be used for modifying the non-CDR loop, e.g., site-directed mutagenesis, random mutagenesis, insertion of at least one amino acid that is foreign to the non-CDR loop amino acid sequence, amino acid substitution. An antigen peptide is inserted into a non-CDR loop, in some examples. In some examples, an antigenic peptide is substituted for the non-CDR loop. The modification, to generate an antigen binding site, is in some cases in only one non-CDR loop. In other instances, more than one non-CDR loop are modified. For instance, the modification is in any one of the non-CDR loops shown in FIG. 1, i.e., AB, CC′, C″ D, EF, and D-E. In some cases, the modification is in the DE loop. In other cases, the modifications are in all four of AB, CC′, C″-D, E-F loops. In certain examples, the binding moieties described herein are bound to the immunoglobulin molecules, e.g., a target antigen binding domain, the non-immunoglobulin binding molecules via their AB, CC′, C″ D, or EF loop and are bound to a bulk-serum protein, such as albumin, via their B-C, C′-C″, or F-G loop. In certain examples, the binding moiety of the first embodiment is bound to the target antigen binding domain via its AB, CC′, C″ D, and EF loop and is bound to a bulk-serum protein, such as albumin, via its BC, C′C″, and FG loop.

In certain examples, the binding moiety of the first embodiment is bound to a target antigen binding domain via one or more of AB, CC′, C″ D, and E-F loop and is bound to a bulk-serum protein, such as albumin, via one or more of BC, C′C″, and FG loop. In certain examples, the binding moiety of the first embodiment is bound to a bulk serum protein, such as albumin, via its AB, CC′, C″ D, or EF loop and is bound to a target antigen binding domain via its BC, C′C″, or FG loop. In certain examples, the binding moiety of the first embodiment is bound to a bulk serum protein, such as albumin, via its AB, CC′, C″ D, and EF loop and is bound to a target antigen binding domain via its BC, C′C″, and FG loop. In certain examples, the binding moiety of the first embodiment is bound to a bulk serum protein, such as albumin, via one or more of AB, CC′, C″ D, and E-F loop and is bound to the target antigen binding protein, via one or more of BC, C′C″, and FG loop.

In certain examples, the binding moiety of the second embodiment is bound to a non-immunoglobulin molecule via one or more of AB, CC′, C″ D, and E-F loop and is bound to a bulk-serum protein, such as albumin, via one or more of BC, C′C″, and FG loop. In certain examples, the binding moiety of the second embodiment is bound to a bulk serum protein, such as albumin, via its AB, CC′, C″ D, or EF loop and is bound to a non-immunoglobulin molecule via its BC, C′C″, or FG loop. In certain examples, the binding moiety of the second embodiment is bound to a bulk serum protein, such as albumin, via its AB, CC′, C″ D, and EF loop and is bound to a non-immunoglobulin molecule via its BC, C′C″, and FG loop. In certain examples, the binding moiety of the second embodiment is bound to a bulk serum protein, such as albumin, via one or more of AB, CC′, C″ D, and E-F loop and is bound to a non-immunoglobulin molecule, via one or more of BC, C′C″, and FG loop.

The binding moieties are any kinds of polypeptides. For example, in certain instances the binding moieties are natural peptides, synthetic peptides, or fibronectin scaffolds, or engineered bulk serum proteins. The bulk serum protein comprises, for example, albumin, fibrinogen, or a globulin. In some embodiments, the binding moieties are engineered scaffolds. Engineered scaffolds comprise, for example, sdAb, a scFv, a Fab, a VHH, a fibronectin type III domain, immunoglobulin-like scaffold (as suggested in Halaby et al., 1999. Prot Eng 12(7):563-571), DARPin, cystine knot peptide, lipocalin, three-helix bundle scaffold, protein G-related albumin-binding module, or a DNA or RNA aptamer scaffold.

In some cases, the binding moiety of the first embodiment binds to at least one target antigen binding domain. In further embodiments, the non-CDR loops within the binding moiety of the first embodiment provide a binding site for the at least one target antigen binding domain. The target antigen binding domain, in some cases, binds to target antigens expressed on the surface of a diseased cell or tissue, for example a tumor or a cancer cell. Target antigens include but are not limited to EpCAM, EGFR, HER-2, HER-3, c-Met, FoIR, PSMA, CD38, BCMA, and CEA. 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

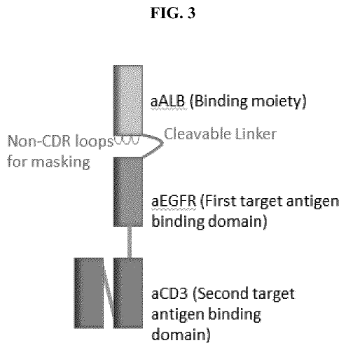

In some cases, the binding moiety of the first embodiment is bound to a first target antigen binding domain via its non-CDR loops and the first target antigen binding domain is further connected to a second target antigen binding domain. Examples of first and second target antigen binding domains include, but are not limited to, a T cell engager, a bispecific T cell engager, a dual-affinity re-targeting antibody, a variable heavy domain (VH), a variable light domain (VL), a scFv comprising a VH and a VL domain, a soluble TCR fragment comprising a Valpha and Vbeta domain, a single domain antibody (sdAb), or a variable domain of camelid derived nanobody (VHH), a non-Ig binding domain, i.e., antibody mimetic, such as anticalins, affilins, affibody molecules, affimers, affitins, alphabodies, avimers, DARPins, fynomers, kunitz domain peptides, and monobodies, a ligand or peptide. In some examples, the first or the second target antigen binding domain is a VHH domain. In some examples, the first or the second target antigen binding domain is a sdAb. In some instances, the first target antigen binding domain is specific for a tumor antigen, such as EGFR, and the second target antigen binding domain is specific for CD3. The binding of the first target antigen binding domain to its target, e.g., a tumor antigen such as EGFR, is masked by the binding moiety of the first embodiment, via its non-CDR loops. One exemplary conditionally active protein, comprising a binding moiety according to the first embodiment, is shown in FIG. 3.

In some cases, the non-CDR loops within the binding moiety of the second embodiment provide a binding site for a non-immunoglobulin binding molecule.

In some cases, the binding moieties comprise a binding site for a bulk serum protein. In some embodiments, the CDRs within the binding moieties provide a binding site for the bulk serum protein. The bulk serum protein is, in some examples, a globulin, albumin, transferrin, IgG1, IgG2, IgG4, IgG3, IgA monomer, Factor XIII, Fibrinogen, IgE, or pentameric IgM. In some embodiments, the binding moieties comprise a binding site for an immunoglobulin light chain. In some embodiments, the CDRs provide a binding site for the immunoglobulin light chain. The immunoglobulin light chain is, in some examples, an IgK free light chain or an Ig, free light chain.

In some examples, the binding moieties comprise any type of binding domain, including but not limited to, domains from a monoclonal antibody, a polyclonal antibody, a recombinant antibody, a human antibody, a humanized antibody. In some embodiments, the binding moiety is a single chain variable fragment (scFv), a soluble TCR fragment, a single-domain antibody such as a heavy chain variable domain (VH), a light chain variable domain (VL) and a variable domain (VHH) of camelid derived nanobody. In other embodiments, the binding moieties are non-Ig binding domains, i.e., antibody mimetic, such as anticalins, affilins, affibody molecules, affimers, affitins, alphabodies, avimers, DARPins, fynomers, kunitz domain peptides, and monobodies.

TABLE 1
Exemplary Sequences for Masking Sequences within the Binding Moieties of this
Disclosure are Provided In SEQ ID Nos. 259-301 And 795.

| MaskID | Sequence | Alt. Names | SEQ ID No. |
|---|---|---|---|
| MASK001 | APGKG | WT | SEQ ID No. 795 |
| MASK002 | GGQDGNEEMGGG | CC12 | SEQ ID No. 259 |
| MASK004 | GGQDGNEEGG | CC10 | SEQ ID No. 260 |
| MASK006 | GGGGQDGNEEMGGGGG | CC16 | SEQ ID No. 261 |
| MASK007 | APFGSEM | | SEQ ID No. 262 |
| MASK008 | AWNGPYE | | SEQ ID No. 263 |
| MASK009 | AQDNGDTKTG | | SEQ ID No. 264 |
| MASK010 | AEAKETQG | | SEQ ID No. 265 |
| MASK011 | ATRREQVEG | | SEQ ID No. 266 |
| MASK012 | AQAPSQP | | SEQ ID No. 267 |
| MASK013 | ATRSRTRNDG | | SEQ ID No. 268 |
| MASK014 | ADVDAPDGLG | | SEQ ID No. 269 |
| MASK015 | AADISDPGG | | SEQ ID No. 270 |
| MASK016 | ALSVDPSG | | SEQ ID No. 271 |
| MASK017 | ARLSVDPG | | SEQ ID No. 272 |
| MASK018 | AVEAADRG | | SEQ ID No. 273 |
| MASK020 | GGPDGNEEMGGG | CC12-Q1P | SEQ ID No. 274 |
| MASK021 | GGFDGNEEMGGG | CC12-Q1F | SEQ ID No. 275 |
| MASK022 | GGGDGNEEMGGG | CC12-Q1G | SEQ ID No. 276 |
| MASK023 | GGEMDGEGQNGG | CC12-scramble | SEQ ID No. 277 |
| MASK024 | GGGGGPDGNEEPGG | | SEQ ID No. 278 |
| MASK025 | GGGGSLDGNEEPGG | | SEQ ID No. 279 |
| MASK026 | GGGGALDGNEEPGG | | SEQ ID No. 280 |
| MASK027 | GGGGGLDGNEEPGG | | SEQ ID No. 281 |
| MASK028 | GGGALDGNEEPGG | | SEQ ID No. 282 |
| MASK029 | GGGGGPDGNEEPGGG | | SEQ ID No. 283 |
| MASK030 | GGSGALDGNEEPGG | | SEQ ID No. 284 |
| MASK031 | GGSGSLDGNEEPGG | | SEQ ID No. 285 |
| MASK038 | GGSGGPDGNEEPGG | | SEQ ID No. 286 |
| MASK039 | GGVRDGPDGNEEPGG | | SEQ ID No. 287 |
| MASK040 | GGSGGPDGNEEPGGGG | | SEQ ID No. 288 |
| MASK041 | GGGRGPDGNEEPGG | | SEQ ID No. 289 |
| MASK042 | GGSGGLDGNEEPGG | | SEQ ID No. 290 |
| MASK043 | GGGVGPDGNEEPGG | | SEQ ID No. 291 |
| MASK044 | GGGEGPDGNEEPGG | | SEQ ID No. 292 |
| MASK046 | GGGVALDGNEEPGG | | SEQ ID No. 293 |
| MASK047 | GGGRALDGNEEPGG | | SEQ ID No. 294 |
| MASK048 | GGYAGLDGNEEPGG | | SEQ ID No. 295 |
| MASK049 | GGAGGPDGNEEPGG | | SEQ ID No. 296 |
| MASK051 | GGRGGPDGNEEPGG | | SEQ ID No. 297 |
| MASK052 | GGGGPDGNEEPGGGG | | SEQ ID No. 298 |
| MASK053 | GGGEALDGNEEPGG | | SEQ ID No. 299 |
| MASK054 | GGDASLDGNEEPGG | | SEQ ID No. 300 |
| MASK055 | GGRDAPDGNEEGG | | SEQ ID No. 301 |

It is contemplated herein that in some embodiments of this disclosure the binding moieties described herein comprise at least one cleavable linker. In one aspect, the cleavable linker comprises a polypeptide having a sequence recognized and cleaved in a sequence-specific manner. The cleavage, in certain examples, is enzymatic, based on pH sensitivity of the cleavable linker, or by chemical degradation.

In some embodiments, the cleavable linker, for example, comprises a protease cleavage site that is not cleavable by an endogenous protease (e.g., an endogenous protease from a T cell). In some instances, the endogenous protease is MMP9. In some instances, the cleavable site is a selective substrate for MMP2, MMP7, or a combination thereof. The cleavable linker, in certain instances, is cleaved only in a tumor microenvironment. Thus, the binding moiety, connected to the cleavable linker, and further bound to the target antigen binding domain, in some examples, maintains the target antigen binding domain in an inert state in circulation until the cleavable linker is cleaved off in a tumor microenvironment. In some embodiments, the binding moiety binds to the target antigen binding domain. In some embodiments, a non-CDR loop provides a binding site for binding of the moiety to the target antigen binding domain. In some embodiments, the binding moiety masks binding of the target binding domain to the target antigen, e.g., via steric occlusion, via specific intramolecular interactions, such as interactions within the different domains of the polypeptide comprising the binding moiety. In some embodiments, the binding moiety further comprises complimentary determining regions (CDRs). In some embodiments, the linker is not cleavable by activated T cells.

In some embodiments, the cleavable linker is not cleavable by an endogenous protease in-vitro. In some embodiments, the endogenous protease is MMP9. In some embodiments, the cleavable linker is cleavable by an endogenous protease in-vitro. In some embodiments, the endogenous protease is MMP7. In some embodiments, the cleavable linker is cleavable by MMP7 in vitro but not cleavable by MMP9 in vitro. In some embodiments, the linker is L077 e.g., (SEQ ID NO: 1095). In some embodiments, the linker is L276 e.g., (SEQ ID NO: 1119). In some embodiments, the linker may further comprise an amino acid sequence selected from G4S or G4T at the N terminal. In some embodiments, the linker may further comprise an amino acid sequence selected from G4S or G4T at the C terminal. In some embodiments, the linker may comprise an amino acid sequence selected from G4S or G4T at the N terminal and may further an amino acid sequence selected from G4S or G4T at the C terminal. In some embodiments, the linker may further comprise an G4S amino acid sequence at the N terminal. In some embodiments, the linker may further comprise an G4S amino acid sequence at the C terminal. In some embodiments, the linker may comprise an G4S amino acid sequence at the N terminal and may further an G4S amino acid sequence at the C terminal.

In some embodiments, the linker sequence lacks an arginine. In some embodiments, the arginine is substituted by glycine. In some embodiments, a ProTriTAC molecule comprising a linker lacking an arginine exhibit improved folding and stability of compared to a ProTriTAC molecule comprising a linker comprising an arginine. In some embodiments, a linker lacking an arginine improve expression of a ProTriTAC molecule compared to a ProTriTAC molecule comprising a linker comprising an arginine. In some embodiments, a linker lacking an arginine improve bioactivity of a ProTriTAC molecule compared to a ProTriTAC molecule comprising a linker comprising an arginine. In some embodiments, a ProTriTAC molecule comprising a linker lacking an arginine exhibit greater serum stability compared to a comparable ProTriTAC molecule comprising a linker comprising an arginine. In some embodiments, a ProTriTAC molecule comprising a linker lacking an arginine exhibit is more stable in a cell culture, e.g., CHO cell culture compared to a comparable ProTriTAC molecule comprising a linker comprising an arginine. In some embodiments, a ProTriTAC molecule comprising a linker lacking an arginine has improved manufacturing compared to a comparable ProTriTAC molecule comprising a linker comprising an arginine. In some embodiments, a ProTriTAC molecule comprising a linker lacking an arginine is more stable compared to a comparable ProTriTAC molecule comprising a linker comprising an arginine when the ProTriTAC molecules are expressed in a T cell. In some embodiments, the linker lacks a serine protease.

In some embodiments, a ProTriTAC molecule comprising a linker lacking an arginine exhibit superior therapeutic activity, compared to a comparable ProTriTAC molecule comprising a linker comprising an arginine. In some embodiments a ProTriTAC molecule comprising a linker lacking an arginine exhibit more stability compared to a comparable ProTriTAC molecule comprising a linker comprising an arginine. In some embodiments a ProTriTAC molecule comprising a linker lacking an arginine is less cleavable compared to a comparable ProTriTAC molecule comprising a linker comprising an arginine. In some embodiments a ProTriTAC molecule comprising a linker lacking an arginine exhibit higher titer (mg/ml) compared to a comparable ProTriTAC molecule comprising a linker comprising an arginine.

The conditionally active binding proteins contemplated herein, in some cases, comprise a protease cleavable linker recognized in a sequence-specific manner by a matrix metalloprotease (MMP), for example a MMP9. In some cases, the protease cleavable linker is recognized by a MMP9 comprises a polypeptide having an amino acid sequence PR(S/T)(L/I)(S/T). In some cases, the protease cleavable linker recognized by a MMP9 comprises a polypeptide having an amino acid sequence LEATA. In some cases, the protease cleavable linker is recognized in a sequence-specific manner by a MMP11. In some cases, the protease cleavable linker recognized by a MMP11 comprises a polypeptide having an amino acid sequence GGAANLVRGG (SEQ IN NO: 3). In some cases, the protease cleavable linker is recognized by a protease disclosed in Table 3. In some cases, the protease cleavable linker is recognized by a protease disclosed in Table 3 comprises a polypeptide having an amino acid sequence selected from a sequence disclosed in Table 3 (SEQ ID NOS: 1-42, 53, and 58-62). In some cases, the protease cleavable linker comprises a polypeptide having an amino acid sequence selected from a sequence disclosed in Table 16 (SEQ ID NOS: 11, 53, 58-60, 62, 909-996). In some cases, the cleavable linker has an amino acid sequence as set forth in SEQ ID No. 59. In some cases, the cleavable linker is recognized by MMP9, matriptase, Urokinase plasminogen activator (uPA) and has an amino acid sequence as set forth in SEQ ID No. 59.

The cleavability of linker sequences by different proteases can be determined using an in vitro assay that shows the percentage of linkers cleaved. In some cases, the percentage cleaved was calculated after incubating the molecules at 37° C. for 1 hr. If the percentage of linkers cleaved under this condition for a certain protease is above zero, then this protease cleavage site is recognized by the corresponding protease.

In some cases, the cleavable linker comprises a protease cleavage site that is recognized by at least one protease selected from the group consisting of: Neutrophil elastase, MMP-12, and MMP-13, and wherein the protease cleavage site is not recognized by furin. In some cases, the protease cleavage site is further recognized by at least one protease selected from the group consisting of: a matriptase and a Urokinase-type Plasminogen Activator (uPA). In some cases, the protease cleavage site is recognized by a matriptase but not by a uPA. In some cases, the protease cleavage site is not recognized by at least one protease selected from the group consisting of: a matriptase and a uPA. In some cases, the protease cleavage site is recognized by at least one protease selected from the group consisting of: MMP-2, MMP-7, MMP-8, and MMP-9. In some cases, the protease cleavage site is recognized by at least one protease selected from the group consisting of: MMP-1, MMP-2, MMP-8, and MMP-9. In some cases, the protease cleavage site is recognized by at least one protease selected from the group consisting of: MMP-2 and MMP-9. In some cases, the protease cleavage site is recognized by at least one protease selected from the group consisting of: MMP-2, MMP-7, and MMP-9. In some cases, the protease cleavage site is recognized by at least one protease selected from the group consisting of: MMP-2, MMP-8, and MMP-9. In some cases, the protease cleavage site is recognized by at least one protease selected from the group consisting of: MMP-2 and MMP-7.

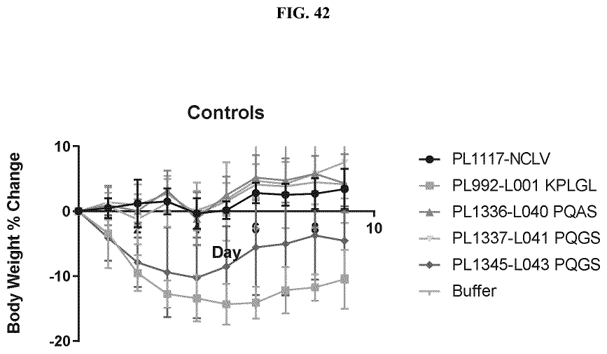

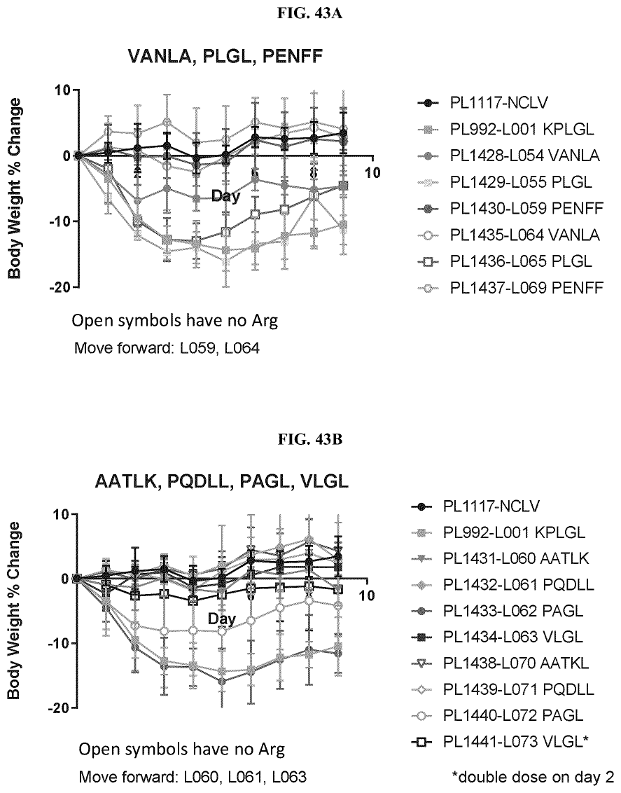

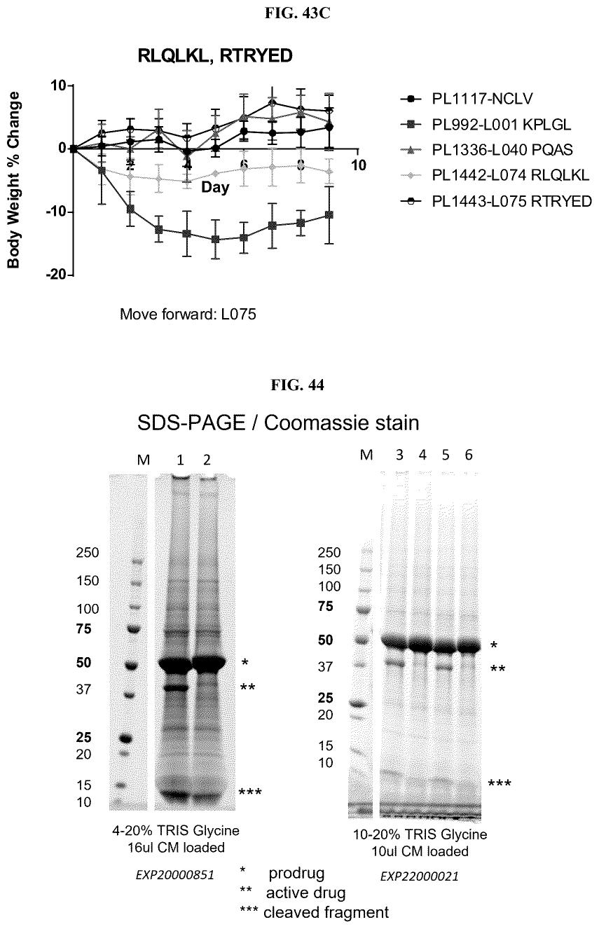

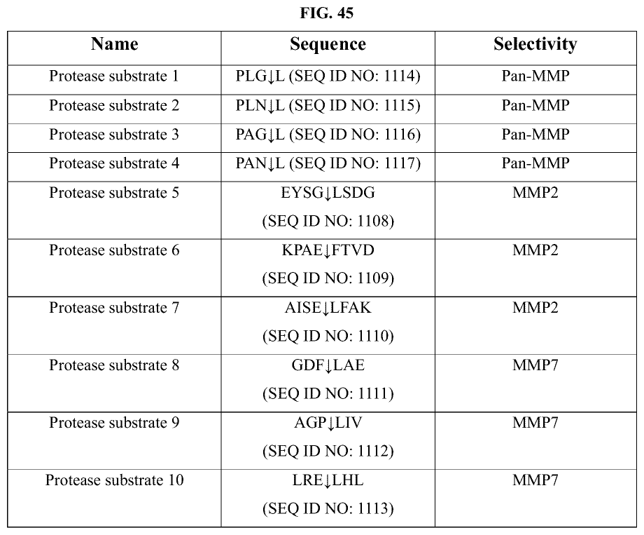

In some cases, the cleavable linkers are less toxic than others. A panel toxicity experiment was performed using control linkers and EGFR-targeting ProTriTAC molecules, and the percentage of change in body weight in mice was monitored, less body weight loss in mice indicates less toxicity. As shown in FIG. 42, linkers L040 and L041 show less body weight loss compared to controls. As shown in FIG. 43A-C, linkers L059, L060, L061, L063, L064, L069, L073 and L075 show less body weight loss compared to other linkers. Linkers with less toxicity were selected for the next efficacy study in EpCAM-targeting ProTriTAC molecules.

In some embodiments of this disclosure the binding moieties described herein comprise at least one non-cleavable linker. The non-cleavable linker comprises, in some examples, a sequence as set forth in SEQ ID No. 51, SEQ ID No. 302, SEQ ID No. 303, SEQ ID No. 304, or SEQ ID No. 305.

TABLE 2
Exemplary Non-cleavable Linker Sequences

| Non-cleavable | | |
|---|---|---|
| Linker ID | Sequence | SEQ ID No. |
| L002 | SGGGGSGGVV | SEQ ID No. 302 |
| L016 | SGGGGSGGGGSGGGGS | SEQ ID No. 303 |
| L017 | SGGGGSGGGGSGGGGGS | SEQ ID No. 304 |
| L046 | SGGGGSGGGS | SEQ ID No. 305 |

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, cathepsins, Cathepsin B, Cathepsin C, Cathepsin D, Cathepsin E, Cathepsin K, Cathepsin L, kallikreins, hK1, hK10, hK15, plasmin, collagenase, Type IV collagenase, stromelysin, Factor Xa, chymotrypsin-like protease, trypsin-like protease, elastase-like protease, subtilisin-like protease, actinidain, bromelain, calpain, caspases, caspase-3, Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP7, MMP8, MMP9, MMP13, MMP11, MMP14, urokinase plasminogen activator (uPA), enterokinase, prostate-specific antigen (PSA, hK3), interleukin-1β converting enzyme, thrombin, FAP (FAP-α), dipeptidyl peptidase, type II transmembrane serine proteases (TTSP), neutrophil serine protease, cathepsin G, proteinase 3, neutrophil serine protease 4, mast cell chymase, and mast cell tryptases.

In some embodiments, the protease cleavage site is recognized by a serine protease, a cysteine protease, an aspartate protease, a threonine protease, a glutamic acid protease, a metalloproteinase, a gelatinase, or a asparagine peptide lyase, and the protease is not an endogenous protease (e.g., an endogenous protease from a T cell). In some instances, the endogenous protease is MMP9. In some instances, the cleavable site is a selective substrate for MMP2, MMP7, or a combination thereof. In some embodiments, the protease cleavage site is recognized by a Cathepsin B, a Cathepsin C, a Cathepsin D, a Cathepsin E, a Cathepsin K, a Cathepsin L, a kallikrein, a hK1, a hK10, a hK15, a plasmin, a collagenase, a Type IV collagenase, a stromelysin, a Factor Xa, a chymotrypsin-like protease, a trypsin-like protease, a elastase-like protease, a subtilisin-like protease, an actinidain, a bromelain, a calpain, a caspase, a caspase-3, a Mir1-CP, a papain, a HIV-1 protease, a HSV protease, a CMV protease, a chymosin, a renin, a pepsin, a matriptase, a legumain, a plasmepsin, a nepenthesin, a metalloexopeptidase, a metalloendopeptidase, a matrix metalloprotease (MMP), a MMP1, a MMP2, a MMP3, a MMP7, a MMP8, a MMP10, a MMP11, a MMP12, a MMP13, a MMP14, an ADAM10, an ADAM12, an urokinase plasminogen activator (uPA), an enterokinase, a prostate-specific target (PSA, hK3), an interleukin-1o converting enzyme, a thrombin, a FAP (FAP-α), a type II transmembrane serine protease (TTSP), a neutrophil elatase, a cathepsin G, a proteinase 3, a neutrophil serine protease 4, a mast cell chymase, a mast cell tryptase, a dipeptidyl peptidase, or a dipeptidyl peptidase IV (DPPIV/CD26). In some embodiments, the target antigen binding domains comprises an sdAb, a scFv, a Fab, a variable heavy chain domain (VHH), or a combination thereof.

TABLE 3
Exemplary Proteases and Protease Recognition
Sequences

| | Cleavage Domain | SEQ ID |
|---|---|---|
| Protease | Sequence | NO: |
| MMP7 | KRALGLPG | 1 |
| MMP7 | (DE)₈RPLALWRS(DR)₈ | 2 |
| MMP9 | PR(S/T)(L/I)(S/T) | 3 |
| MMP9 | LEATA | 4 |
| MMP11 | GGAANLVRGG | 5 |
| MMP14 | SGRIGFLRTA | 6 |
| MMP | PLGLAG | 7 |
| MMP | PLGLAX | 8 |
| MMP | PLGC(me)AG | 9 |
| MMP | ESPAYYTA | 10 |
| MMP | RLQLKL | 11 |
| MMP | RLQLKAC | 12 |
| MMP2, MMP9, MMP14 | EP(Cit)G(Hof) YL | 13 |
| Urokinase plasminogen | SGRSA | 14 |
| activator (uPA) | | |
| Urokinase plasminogen | DAFK | 15 |
| activator (uPA) | | |
| Urokinase plasminogen | GGGRR | 16 |
| activator (uPA) | | |
| Lysosomal Enzyme | GFLG | 17 |
| Lysosomal Enzyme | ALAL | 18 |
| Lysosomal Enzyme | FK | 19 |
| Cathepsin B | NLL | 20 |
| Cathepsin D | PIC(Et)FF | 21 |
| Cathepsin K | GGPRGLPG | 22 |
| Prostate Specific | HSSKLQ | 23 |
| Antigen | | |
| Prostate Specific | HSSKLQL | 24 |
| Antigen | | |
| Prostate Specific | HSSKLQEDA | 25 |
| Antigen | | |
| Herpes Simplex Virus | LVLASSSFGY | 26 |
| Protease | | |
| HIV Protease | GVSQNYPIVG | 27 |
| CMV Protease | GVVQASCRLA | 28 |
| Thrombin | F(Pip)RS | 29 |
| Thrombin | DPRSFL | 30 |
| Thrombin | PPRSFL | 31 |
| Caspase-3 | DEVD | 32 |
| Caspase-3 | DEVDP | 33 |
| Caspase-3 | KGSGDVEG | 34 |
| Interleukin 1ß | GWEHDG | 35 |
| converting enzyme | | |
| Enterokinase | EDDDDKA | 36 |
| FAP | KQEQNPGST | 37 |
| Kallikrein 2 | GKAFRR | 38 |
| Plasmin | DAFK | 39 |
| Plasmin | DVLK | 40 |
| Plasmin | DAFK | 41 |
| TOP | ALLLALL | 42 |
| MMP9 + matriptase | KPLGLQARVV | 58 |
| MMP9 + matriptase + | PQASTGRSGG | 59 |
| uPA | | |
| MMP9 + matriptase + | PQGSTGRAAG | 60 |
| uPA | | |
| Matriptase + uPA | PPASSGRAGG | 61 |
| MMP9 + matriptase | PIPVQGRAH | 62 |
| MMP9 + matriptase | PQGSTARSAG | 909 |

Proteases are known to be secreted by some diseased cells and tissues, for example tumor or cancer cells, creating a microenvironment that is rich in proteases or a protease-rich microenvironment. In some case, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen binding proteins to have additional specificity because T cells will not be bound by the antigen binding protein except in the protease rich microenvironment of the targeted cells or tissue.

The binding moiety comprising the cleavable linker thus masks the binding of a first or a second target antigen binding domain to their respective targets. In some embodiments, the binding moiety is bound to a first target antigen binding domain, which is further bound to a second target antigen binding domain, in the following order: binding moiety (M): cleavable linker (L): first target antigen binding domain (T1): second antigen binding domain (T2). In other examples, the domains are organized in any one of the following orders: M:L:T2:T1; T2:T1:L:M, T1:T2:L:M. The binding moiety is further bound to a half-life extending protein, such as albumin or any other of its targets as described below. In some instances, the binding moiety is albumin or comprises a binding site for albumin. In some instances the binding moiety comprises a binding site for IgE. In some embodiments, the binding moiety comprises a binding site for Igκ free light chain.

TABLE 4
Exemplary sequences for the binding moieties
comprising a cleavable linker are provided in
SEQ ID Nos. 796-800.
Sequence

SEQ ID No. 796
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSSGGGGKPLGLQARVVGGGGT
SEQ ID No. 797
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSS
GGGGPQASTGRSGGGGGGT
SEQ ID No. 798
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSS
GGGGPQGSTGRAAGGGGGT
SEQ ID No. 799
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSS
GGGGPPASSGRAGGGGGT
SEQ ID No. 800
EVQLVESGGGLVQPGNSLRLSCAASGFTFSKFGMSWVRQGGGGGLDGNE
EPGGLEWVSSISGSGRDTLYADSVKGRFTISRDNAKTTLYLQMNSLRPE
DTAVYYCTIGGSLSVSSQGTLVTVSS
GGGGPIPVQGRAHGGGGT

### Targets of Conditionally Active Binding Proteins

The conditionally active binding proteins described herein are activated by cleavage of the at least one cleavable linker attached to the binding moieties within said conditionally active proteins. It is contemplated that in some cases the activated binding protein binds to a target antigen involved in and/or associated with a disease, disorder or condition. In particular, target antigens associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease are contemplated to be the target for the activated binding proteins disclosed herein.

In some embodiments, the target antigen is a tumor antigen expressed on a tumor cell. Tumor antigens are well known in the art and include, for example, EpCAM, EGFR, HER-2, HER-3, c-Met, FoIR, PSMA, CD38, BCMA, and CEA. 5T4, AFP, B7-H3, CDH-6, CAIX, CD117, CD123, CD138, CD166, CD19, CD20, CD205, CD22, CD30, CD33, CD352, CD37, CD44, CD52, CD56, CD70, CD71, CD74, CD79b, DLL3, EphA2, FAP, FGFR2, FGFR3, GPC3, gpA33, FLT-3, gpNMB, HPV-16 E6, HPV-16 E7, ITGA2, ITGA3, SLC39A6, MAGE, mesothelin, Muc1, Muc16, NaPi2b, Nectin-4, CDH-3, CDH-17, EPHB2, ITGAV, ITGB6, NY-ESO-1, PRLR, PSCA, PTK7, ROR1, SLC44A4, SLITRK5, SLITRK6, STEAP1, TIM1, Trop2, or WT1.

In some embodiments, the target antigen is an immune checkpoint protein. Examples of immune checkpoint proteins include but are not limited to CD27, CD137, 2B4, TIGIT, CD155, ICOS, HVEM, CD40L, LIGHT, OX40, DNAM-1, PD-L1, PD1, PD-L2, CTLA-4, CD8, CD40, CEACAM1, CD48, CD70, A2AR, CD39, CD73, B7-H3, B7-H4, BTLA, IDO1, IDO2, TDO, KIR, LAG-3, TIM-3, or VISTA.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, inflamed or fibrotic tissue cell.

In some embodiments, the target antigen comprises an immune response modulator that is not a cytokine. Examples of immune response modulator include but are not limited to B7-1 (CD80), B7-2 (CD86), CD3, or GITR.

In some embodiments, the first target antigen binding domain or the second target antigen binding domain comprises an anti-EGFR domain, an anti-EpCAM domain, an anti-DLL3 domain, an anti-MSLN domain, an anti-PSMA domain, an anti-BDMA domain, or any combinations thereof. In some embodiments, the first target antigen binding domain or the second target antigen binding domain comprises an anti-EGFR sdAb, an anti-EpCAM sdAb, an anti-DLL3 sdAb, an anti-MSLN sdAb, an anti-PSMA sdAb, an anti-BDMA sdAb, or any combinations thereof.

In some embodiments, an anti-EGFR domain of this disclosure comprises an amino acid selected from the group consisting of SEQ ID Nos. 55, and 737-785. In some embodiments, an anti-PSMA domain of this disclosure comprises an amino acid selected from the group consisting of SEQ ID Nos. 57-73. In some embodiments, an anti-BCMA domain of this disclosure comprises an amino acid selected from the group consisting of SEQ ID Nos. 91-214.

In some embodiments, an anti-MSLN domain of this disclosure comprises an amino acid selected from the group consisting of SEQ ID Nos. 215-258. In some embodiments, an anti-DLL3 domain of this disclosure comprises an amino acid selected from the group consisting of SEQ ID Nos. 306-736. In some embodiments, an anti-EpCAM domain of this disclosure comprises an amino acid selected from the group consisting of SEQ ID Nos. 804-841.

In some embodiments, the first target antigen binding domain or the second target antigen binding domain comprises an anti-CD3 domain. In some embodiments, the anti-CD3 domain comprises an anti-CD3 scFV. In some embodiments, the anti-CD3 scFv comprises an amino acid sequence selected from the group consisting of: SEQ ID Nos. 74-90, and 794.

### Binding Protein Variants

As used herein, the term “binding protein variants” refers to variants and derivatives of the conditionally active target-binding proteins described herein, containing a binding moiety as described above, comprising non-CDR loops that bind to an immunoglobulin binding molecule, such as a first or a second target antigen binding domain, a non-immunoglobulin binding molecule. In certain embodiments, amino acid sequence variants of the conditionally active target-binding proteins described herein are contemplated. For example, in certain embodiments amino acid sequence variants of the conditionally active target-binding proteins described herein are contemplated to improve the binding affinity and/or other biological properties of the binding proteins. Exemplary method for preparing amino acid variants include, but are not limited to, introducing appropriate modifications into the nucleotide sequence encoding the antibody, or by peptide synthesis. Such modifications include, for example, deletions from, and/or insertions into and/or substitutions of residues within the amino acid sequences of the antibody.

Any combination of deletion, insertion, and substitution can be made to the various domains to arrive at the final construct, provided that the final construct possesses the desired characteristics, e.g., antigen-binding. In certain embodiments, binding protein variants having one or more amino acid substitutions are provided. Sites of interest for substitution mutagenesis include the CDRs and framework regions. Amino acid substitutions may be introduced into the variable domains of a conditionally active protein of interest and the products screened for a desired activity, e.g., retained/improved antigen binding, decreased immunogenicity, or improved antibody-dependent cell mediated cytotoxicity (ADCC) or complement dependent cytotoxicity (CDC). Both conservative and non-conservative amino acid substitutions are contemplated for preparing the antibody variants.

In another example of a substitution to create a variant conditionally active antibody, one or more hypervariable region residues of a parent antibody are substituted. In general, variants are then selected based on improvements in desired properties compared to a parent antibody, for example, increased affinity, reduced affinity, reduced immunogenicity, increased pH dependence of binding. For example, an affinity matured variant antibody can be generated, e.g., using phage display-based affinity maturation techniques such as those described herein and known in the field.

In another example, substitutions are made in hypervariable regions (HVR) of a parent conditionally active antibody to generate variants and variants are then selected based on binding affinity, i.e., by affinity maturation. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. Substitutions can be in one, two, three, four, or more sites within a parent antibody sequence.

In some embodiments, a conditionally active binding protein, as described herein comprises a VL domain, or a VH domain, or both, with amino acid sequences corresponding to the amino acid sequence of a naturally occurring VL or VH domain, respectively, but that has been “humanized”, i.e., by replacing one or more amino acid residues in the amino acid sequence of said naturally occurring VL or VH domains (and in particular in the framework sequences) by one or more of the amino acid residues that occur at the corresponding position(s) in a VL or VH domain from a conventional 4-chain antibody from ahuman being (e.g., as indicated above). This can be performed in a manner known in the field, which will be clear to the skilled person, for example on the basis of the further description herein. Again, it should be noted that such humanized conditionally active target-binding antibodies of the disclosure are obtained in any suitable manner known per se and thus are not strictly limited to polypeptides that have been obtained using a polypeptide that comprises a naturally occurring VL and/or VH domain as a starting material. In some additional embodiments, an conditionally active target-binding antibody, as described herein, comprises a VL and a VH domain with amino acid sequences corresponding to the amino acid sequence of a naturally occurring VL or VH domain, respectively, but that has been “camelized”, i.e., by replacing one or more amino acid residues in the amino acid sequence of a naturally occurring VL or VH domain from a conventional 4-chain antibody by one or more of the amino acid residues that occur at the corresponding position(s) in a VL or a VH domain of a heavy chain antibody. Such “camelizing” substitutions are preferably inserted at amino acid positions that form and/or are present at the VH-VL interface, and/or at the so-called Camelidae hallmark residues (see for example WO 94/04678 and Davies and Riechmann (1994 and 1996)). Preferably, the VH sequence that is used as a starting material or starting point for generating or designing the camelized single domain is preferably a VH sequence from a mammal, more preferably the VH sequence of a human being, such as a VH3 sequence. However, it should be noted that such camelized conditionally active antibodies of the disclosure, in certain embodiments, are obtained in any suitable manner known in the field and thus are not strictly limited to polypeptides that have been obtained using a polypeptide that comprises a naturally occurring VL and/or VH domain as a starting material. For example, both “humanization” and “camelization” is performed by providing a nucleotide sequence that encodes a naturally occurring VL and/or VH domain, respectively, and then changing, one or more codons in said nucleotide sequence in such a way that the new nucleotide sequence encodes a “humanized” or “camelized” conditionally active antibody, respectively. This nucleic acid can then be expressed, so as to provide the desired target-antigen binding capability. Alternatively, in other embodiments, a “humanized” or “camelized” conditionally active antibody is synthesized de novo using known peptide synthesis technique from the amino acid sequence of a naturally occurring antibody comprising a VL and/or VH domain. In some embodiments, a “humanized” or “camelized” conditionally active antibody is synthesized de novo using known peptide synthesis technique from the amino acid sequence or nucleotide sequence of a naturally occurring antibody comprising a VL and/or VH domain, respectively, a nucleotide sequence encoding the desired humanized or camelized conditionally active domain antibody of the disclosure, respectively, is designed and then synthesized de novo using known techniques for nucleic acid synthesis, after which the nucleic acid thus obtained is expressed in using known expression techniques, so as to provide the desired conditionally active antibody of the disclosure.

Other suitable methods and techniques for obtaining the conditionally active binding protein of the disclosure and/or nucleic acids encoding the same, starting from naturally occurring sequences for VL or VH domains for example comprises combining one or more parts of one or more naturally occurring VL or VH sequences (such as one or more framework (FR) sequences and/or complementarity determining region (CDR) sequences), and/or one or more synthetic or semi-synthetic sequences, and/or a naturally occurring sequence for a CH2 domain, and a naturally occurring sequence for a CH3 domain comprising amino acid substitutions that favor formation of heterodimer over homodimer, in a suitable manner, so as to provide a conditionally active binding protein of the disclosure or a nucleotide sequence or nucleic acid encoding the same.

In some embodiments, the disclosure provides a conditionally active chimeric antigen receptor that comprises a single polypeptide chain, comprising a binding moiety comprising a non-CDR loop and a cleavable linker, a target antigen binding domain; a transmembrane domain; and an intracellular signaling domain. In some embodiments, the cleavable linker is not cleavable by an endogenous protease. In some embodiments, the binding moiety is capable of masking the binding of the target antigen binding domain to its target.

### Affinity Maturation

In designing conditionally active binding proteins for therapeutic applications, it is desirable to create proteins that, for example, modulate a functional activity of a target, and/or improved binding proteins such as binding proteins with higher specificity and/or affinity and/or and binding proteins that are more bioavailable, or stable or soluble in particular cellular or tissue environments.

The conditionally active binding proteins described in the present disclosure exhibit improved the binding affinities towards the target, for example a tumor antigen expressed on a cell surface. In some embodiments, the conditionally active binding protein of the present disclosure is affinity matured to increase its binding affinity to the target, using any known technique for affinity-maturation (e.g., mutagenesis, chain shuffling, CDR amino acid substitution). Amino acid substitutions may be conservative or semi-conservative. For example, the amino acids glycine, alanine, valine, leucine and isoleucine can often be substituted for one another (amino acids having aliphatic side chains). Of these possible substitutions, typically glycine and alanine are used to substitute for one another since they have relatively short side chains and valine, leucine and isoleucine are used to substitute for one another since they have larger aliphatic side chains which are hydrophobic. Other amino acids which may often be substituted for one another include but are not limited to: phenylalanine, tyrosine and tryptophan (amino acids having aromatic side chains); lysine, arginine and histidine (amino acids having basic side chains); aspartate and glutamate (amino acids having acidic side chains); asparagine and glutamine (amino acids having amide side chains); and cysteine and methionine (amino acids having sulphur-containing side chains). In some embodiments, the conditionally active target-binding proteins are isolated by screening combinatorial libraries, for example, by generating phage display libraries and screening such libraries for antibodies possessing the desired binding characteristics towards a target antigen, such as a tumor antigen expressed on a cell surface.

### Conditionally Active Binding Protein Modifications

The conditionally active binding proteins described herein encompass derivatives or analogs in which (i) an amino acid is substituted with an amino acid residue that is not one encoded by the genetic code, (ii) the mature polypeptide is fused with another compound such as polyethylene glycol, or (iii) additional amino acids are fused to the protein, such as a leader or secretory sequence or a sequence to block an immunogenic domain and/or for purification of the protein.

Typical modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristylation, oxidation, proteolytic processing, phosphorylation, prenylation, racemization, selenoylation, sulfation, transfer-RNA mediated addition of amino acids to proteins such as arginylation, and ubiquitination.

Modifications are made anywhere in the conditionally active binding proteins described herein, including the peptide backbone, the amino acid side-chains, and the amino or carboxyl termini. Certain common peptide modifications that are useful for modification of the conditionally active binding proteins include glycosylation, lipid attachment, sulfation, gamma-carboxylation of glutamic acid residues, hydroxylation, blockage of the amino or carboxyl group in a polypeptide, or both, by a covalent modification, and ADP-ribosylation.

In some embodiments, the conditionally active binding proteins of the disclosure are conjugated with drugs to form antibody-drug conjugates (ADCs). In general, ADCs are used in oncology applications, where the use of antibody-drug conjugates for the local delivery of cytotoxic or cytostatic agents allows for the targeted delivery of the drug moiety to tumors, which can allow higher efficacy, lower toxicity, etc.

### Polynucleotides Encoding the Binding Moieties or the Conditionally Active Binding Proteins

Also provided, in some embodiments, are polynucleotide molecules encoding the binding moieties as described herein. In some embodiments, the polynucleotide molecules are provided as a DNA construct. In other embodiments, the polynucleotide molecules are provided as a messenger RNA transcript.

Also provided, in some embodiments, are polynucleotide molecules encoding the conditionally active binding proteins as described herein. In some embodiments, the polynucleotide molecules are provided as a DNA construct. In other embodiments, the polynucleotide molecules are provided as a messenger RNA transcript.

The polynucleotide molecules are constructed by known methods such as by combining the genes encoding the various domains (e.g. binding moiety, target antigen binding domain, etc.) either separated by peptide linkers or, in other embodiments, directly linked by a peptide bond, into a single genetic construct operably linked to a suitable promoter, and optionally a suitable transcription terminator, and expressing it in bacteria or other appropriate expression system such as, for example CHO cells. Depending on the vector system and host utilized, any number of suitable transcription and translation elements, including constitutive and conditionally active promoters, may be used. The promoter is selected such that it drives the expression of the polynucleotide in the respective host cell.

In some embodiments, the polynucleotides described herein are inserted into vectors, such as expression vectors, which represent further embodiments. This recombinant vector can be constructed according to known methods. Vectors of particular interest include plasmids, phagemids, phage derivatives, virii (e.g., retroviruses, adenoviruses, adeno-associated viruses, herpes viruses, lentiviruses, and the like), and cosmids.

A variety of expression vector/host systems may be utilized to contain and express the polynucleotide encoding the polypeptide of the described conditionally active binding protein. Examples of expression vectors for expression in E. coli are pSKK (Le Gall et al., J Immunol Methods. (2004) 285(1):111-27) or pcDNA5 (Invitrogen) for expression in mammalian cells.

Thus, the binding moieties or the conditionally active binding proteins comprising the binding moieties as described herein, in some embodiments, are produced by introducing vectors encoding the binding moieties or the binding proteins as described above into host cells and culturing said host cells under conditions whereby the binding moieties or the binding proteins, or domains thereof are expressed.

### Pharmaceutical Compositions

Also provided, in some embodiments, are pharmaceutical compositions comprising a therapeutically effective amount of a conditionally active binding protein of the present disclosure, and at least one pharmaceutically acceptable carrier. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms is, in some cases, ensured by the inclusion of various antibacterial and antifungal agents.

The conditionally active binding proteins described herein are contemplated for use as medicaments. Administration is effected by different ways, e.g., by intravenous, intraperitoneal, subcutaneous, intramuscular, topical or intradermal administration. In some embodiments, the route of administration depends on the kind of therapy and the kind of compound contained in the pharmaceutical composition. The dosage regimen will be determined by the attending physician and other clinical factors. Dosages for any one patient depends on many factors, including the patient's size, body surface area, age, sex, the particular compound to be administered, time and route of administration, the kind of therapy, general health and other drugs being administered concurrently. An “effective dose” refers to amounts of the active ingredient that are sufficient to affect the course and the severity of the disease, leading to the reduction or remission of such pathology and may be determined using known methods.

### Methods of Treatment

Also provided herein, in some embodiments, are methods and uses for stimulating the immune system of an individual in need thereof comprising administration of a conditionally active binding protein as described herein. In some instances, administration induces and/or sustains cytotoxicity towards a cell expressing a target antigen. In some instances, the cell expressing a target antigen is a cancer or tumor cell, a virally infected cell, a bacterially infected cell, an autoreactive T or B cell, damaged red blood cells, arterial plaques, or fibrotic tissue. In some embodiments, the target antigen is an immune checkpoint protein.

Also provided herein are methods and uses for a treatment of a disease, disorder or condition associated with a target antigen comprising administering to an individual in need thereof a conditionally active binding protein as described herein. Diseases, disorders or conditions associated with a target antigen include, but are not limited to, viral infection, bacterial infection, auto-immune disease, transplant rejection, atherosclerosis, or fibrosis. In other embodiments, the disease, disorder or condition associated with a target antigen is a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In some embodiment, the disease, disorder or condition associated with a target antigen is cancer. In some embodiments, the cancer is an EGFR over-expressing cancer. In some embodiments, the cancer is an CTLA4 over-expressing cancer. In some embodiments, the cancer is a cancer exhibiting low expression of a matrix metalloprotease, e.g., MMP. In one instance, the cancer is a hematological cancer. In another instance, the cancer is a melanoma. In a further instance, the cancer is non-small cell lung cancer. In yet further instance, the cancer is breast cancer. In yet further instance, the cancer is an ovarian cancer, e.g., epithelial ovarian cancer.

As used herein, in some embodiments, “treatment” or “treating” or “treated” refers to therapeutic treatment wherein the object is to slow (lessen) an undesired physiological condition, disorder or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In other embodiments, “treatment” or “treating” or “treated” refers to prophylactic measures, wherein the object is to delay onset of or reduce severity of an undesired physiological condition, disorder or disease, such as, for example is a person who is predisposed to a disease (e.g., an individual who carries a genetic marker for a disease such as breast cancer).

In some embodiments of the methods described herein, the conditionally active binding proteins described herein are administered in combination with an agent for treatment of the particular disease, disorder or condition. Agents include but are not limited to, therapies involving antibodies, small molecules (e.g., chemotherapeutics), hormones (steroidal, peptide, and the like), radiotherapies (γ-rays, X-rays, and/or the directed delivery of radioisotopes, microwaves, UV radiation and the like), gene therapies (e.g., antisense, retroviral therapy and the like) and other immunotherapies. In some embodiments, the conditionally active binding proteins described herein are administered in combination with anti-diarrheal agents, anti-emetic agents, analgesics, opioids and/or non-steroidal anti-inflammatory agents. In some embodiments, the conditionally active binding proteins described herein is administered before, during, or after surgery.

In some embodiments, the disclosure provides a kit comprising a ProTriTAC molecule described herein. In some embodiments, the kit is used to treat a disease described herein.

### EXAMPLES

The examples below further illustrate the described embodiments without limiting the scope of the disclosure.

### Example 1: Construction of an Exemplary Binding Moiety which Binds to Albumin and a Target Antigen Binding Domain Whose Target is EGFR

The sequence of an engineered protein scaffold comprising CDR loops capable of binding albumin and non-CDR loops is obtained. Overlapping PCR is used to introduce random mutations in the non-CDR loop regions, thereby generating a library. The resultant sequences are cloned into a phage display vector, thereby generating a phage display library. Escherichia coli cells are transformed with the library and used to construct a phage display library. ELISA is performed using an immobilized target antigen binding domain with specificity for EGFR. A clone with high specificity for EGFR is selected. Affinity maturation is performed by re-randomizing residues in the non-CDR loop regions as before.

Sequence alignment of non-CDR loop regions of the resultant proteins is performed to determine sequence conservation between proteins with high affinity for the EGFR binding target antigen binding domain. Site directed mutagenesis of one or more amino acids within these regions of sequence conservation is performed to generate additional proteins. Binding of the resultant proteins to an immobilized target antigen binding domain whose target is EGFR is measured in an ELISA. A protein with the highest affinity for the target antigen binding domain is selected.

The sequence of this binding moiety is cloned into a vector comprising a sequence for a cleavable linker, and sequences for a second target antigen binding domain that binds to a second target antigen, e.g., CD3. The resultant vector is expressed in a heterologous expression system to obtain a conditionally active target binding protein that comprises a binding moiety comprising a cleavable linker and non-CDR loops which provide a binding site specific for the target antigen binding domain whose target is EGFR, and CDR loops which are specific for albumin.

### Example 2: Construction of an Exemplary Binding Moiety which Binds to Albumin and a Target Antigen Binding Domain Whose Target is CD3

The sequence of an engineered protein scaffold comprising CDR loops capable of binding albumin and non-CDR loops is obtained. Overlapping PCR is used to introduce random mutations in the non-CDR loop regions, thereby generating a library. The resultant sequences are cloned into a phage display vector, thereby generating a phage display library. Escherichia coli cells are transformed with the library and used to construct a phage display library. ELISA is performed using an immobilized target antigen binding domain with specificity for CD3. A clone with high specificity for CD3 is selected. Affinity maturation is performed by re-randomizing residues in the non-CDR loop regions as before.

Sequence alignment of non-CDR loop regions of the resultant proteins is performed to determine sequence conservation between proteins with high affinity for the EGFR binding target antigen binding domain. Site directed mutagenesis of one or more amino acids within these regions of sequence conservation is performed to generate additional proteins. Binding of the resultant proteins to an immobilized target antigen binding domain whose target is CD3 is measured in an ELISA. A protein with the highest affinity for the target antigen binding domain is selected.

The sequence of this binding moiety is cloned into a vector comprising a sequence for a cleavable linker, and sequences for a second target antigen binding domain that binds to a second target antigen, e.g., EGFR. The resultant vector is expressed in a heterologous expression system to obtain a conditionally active target binding protein that comprises a binding moiety comprising a cleavable linker and non-CDR loops which provide a binding site specific for the target antigen binding domain whose target is CD3, and CDR loops which are specific for albumin.

### Example 3: A Conditionally Active Binding Protein of this Disclosure Exhibit Reduced Specificity Toward Cell Line which Overexpresses EGFR but is Protease Deficient

Cells overexpressing EGFR and exhibiting low expression of a matrix metalloprotease are separately incubated with an exemplary conditionally active binding protein according to the present disclosure and a non-conditionally active control binding protein. Cells expressing normal levels of EGFR and proteases are also incubated with a conditionally active binding protein according to the present disclosure and a non-conditionally active control binding protein. Both proteins comprise a target antigen binding domain with specificity toward PSMA.

Results indicate that in the absence of protease secretion, the conditionally active binding protein of the present disclosure interacts with the protease expressing cells but does not interact with the EGFR expressed on the surface of the protease deficient cells. In contrast, the non-conditionally active control binding protein lacks the ability to selectively bind the protease expressing cells over the protease deficient ones. Thus, the exemplary conditionally active binding protein receptor of the present disclosure is advantageous, for example, in terms of reducing off-tumor toxicity.

### Example 4: Treatment with an Exemplary Conditionally Active Binding Protein of the Present Disclosure Inhibits In Vivo Tumor Growth

Murine tumor line CT26 is implanted subcutaneously in Balb/c mice and on day 7 post-implantation the average size of the tumor is measured. Test mice are treated with an exemplary conditionally active binding protein which has a target antigen binding domain specific for CTLA4 and another target antigen binding domain specific for CD3, wherein either the CTLA4 or the CD3 specific domain is bound to a binding moiety via its non-CDR loops, the binding moiety comprises a cleavable linker, and is bound to albumin. Control mice are treated with binding protein that contains CD3/CTLA4 specific domains but do not contain the binding moiety or the cleavable linker, and are not conditionally active. Results show that treatment with the exemplary conditionally active binding protein of the present disclosure inhibits tumor more efficiently than the comparator binding protein which does not contain the moiety with non-CDR loops.

### Example 5: Exemplary Conditionally Active Binding Protein Exhibits Reduced Specificity Toward Cell Line which Overexpresses Antigen but is Protease Deficient

Cells overexpressing CTLA-4 and exhibiting low expression of a matrix metalloprotease are separately incubated with an exemplary CTLA4 specific conditionally active binding protein of this disclosure, containing a binding moiety that binds to a CTLA4 binding domain via its non-CDR loops and albumin via its CDRs; or a control CTLA-4 binding antibody which does not contain the binding moiety which binds to a CTLA4 binding domain via its non-CDR loops and to albumin via its CDRs. Cells expressing normal levels of antigens and proteases are also incubated with the exemplary CTLA4 specific conditionally active binding protein, or the control CTLA4 binding antibody.

Results indicate that in the absence of protease secretion, the conditionally active binding protein of the present disclosure binds the protease expressing cells but does not bind the protease-deficient antigen expressing cells. In contrast, the control antibody lacks the ability to selectively bind the protease expressing cells over the protease deficient ones. Thus, the exemplary conditionally active binding protein of the present disclosure is advantageous, for example, in terms of reducing off-tumor toxicity.

### Example 6: Purification, Pharmacokinetic Analysis, and Potency Assays for Exemplary ProTriTAC Molecules

### A) Expression, Purification and Stability of Exemplary ProTriTAC Molecules

### Protein Production

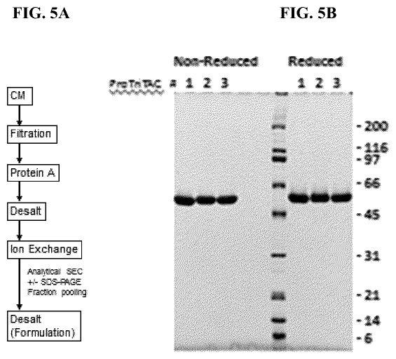

Sequences of exemplary ProTriTAC (also referred to as Pro-trispecific) molecules were cloned into mammalian expression vector pcDNA 3.4 (Invitrogen) preceded by a leader sequence and followed by a 6× Histidine Tag. Expi293F cells (Life Technologies A14527) were maintained in suspension in Optimum Growth Flasks (Thomson) between 0.2 to 8×1e6 cells/ml in Expi 293 media. Purified plasmid DNA was transfected into Expi293 cells in accordance with Expi293 Expression System Kit (Life Technologies, A14635) protocols, and maintained for 4-6 days post transfection. Alternatively, sequences of trispecific molecules were cloned into mammalian expression vector pDEF38 (CMC ICOS) transfected into CHO-DG44 dhfr- cells, stable pools generated, and cultured in production media for up to 12 days prior to purification. The amount of the exemplary trispecific proteins in conditioned media was quantitated using an Octet RED 96 instrument with Protein A tips (ForteBio/Pall) using a control trispecific protein for a standard curve. Conditioned media from either host cell was filtered and partially purified by affinity and desalting chromatography. Trispecific proteins were subsequently polished by ion exchange and upon fraction pooling formulated in a neutral buffer containing excipients. Final purity was assessed by SDS-PAGE and analytical SEC using an Acquity BEH SEC 200 1.7u 4.6×150 mm column (Waters Corporation) resolved in an aqueous/organic mobile phase with excipients at neutral pH on a 1290 LC system and peaks integrated with Chemstation CDS software (Agilent). Trispecific proteins purified from CHO host cells were analyzed by running an SDS-PAGE, as shown in FIG. 5.

### Stability Assessment

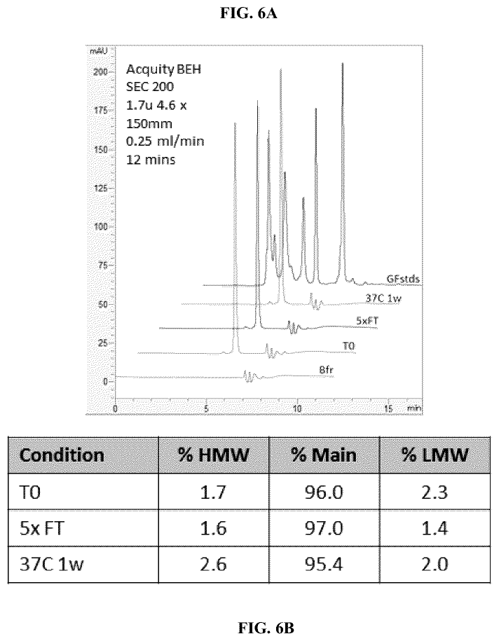

Purified Protrispecific proteins in two formulations were sub-aliquoted into sterile tubes and stressed by five freeze-thaw cycles each comprising greater than 1 hour at −80° C. and room temperature or by incubation at 37° C. for 1 week. Stressed samples were evaluated for concentration and turbidity by UV spectrometry using UV transparent 96 well plates (Corning 3635) with a SpectraMax M2 and SoftMaxPro Software (Molecular Devices), SDS-PAGE, and analytical SEC and compared to the same analysis of control non-stressed samples. An overlay of chromatograms from analytical SEC of control and stressed samples for a single exemplary trispecific ProTriTAC molecule purified from 293 host cells is depicted in FIG. 6.

### B) ProTriTAC Exhibits Potent, Protease-Dependent, Anti-Tumor Activity in a Rodent Tumor Xenograft Model

An exemplary ProTriTAC molecule (SEQ ID NO: 46) containing an EGFR binding domain as the target binding domain, a CD3 binding domain and an albumin binding domain comprising a masking moiety (SEQ ID NO: 50) and a cleavable linker (SEQ ID NO: 53) was evaluated for anti-tumor activity in vivo in an HCT116 subcutaneous xenograft tumor admixed with expanded human T cells in immunocompromised NCG mice. A non-cleavable EGFR targeting ProTriTAC molecule (SEQ ID NO: 47) and a GFP targeting ProTriTAC molecule (SEQ ID NO: 49) were also used in the study. Specifically, 5×10⁶ HCT116 cells were admixed with 2.5×10⁶ expanded T cells per mouse on day 0. Dosing of the test molecules (EGFR targeting ProTriTAC, non-cleavable EGFR targeting Pro-TriTAC, and GFP targeting ProTriTAC) were performed starting on the following day with a q.d.×10 (single daily dose for 10 days) schedule via intraperitoneal injection, at a dose of 0.03 mg/kg. Tumor volumes were determined using caliper measurements and calculated using the formula V=(length×width×width)/2, at the indicated times. Results shown in FIG. 7 indicate that following final dose of test molecules, on day 10, tumor growth was arrested in mice administered the activatable EGFR targeting ProTriTAC molecule. Whereas, administering the GFP targeting ProTriTAC molecule was not effective in inhibiting tumor growth and the administration of the EGFR targeting non-cleavable ProTriTAC molecule was not as potent as the activatable ProTriTAC, in arresting tumor growth.

### C) Demonstration of Functional Masking and Stability of ProTriTAC In Vivo in a Three-Week Cynomolgus Monkey Pharmacokinetic Study

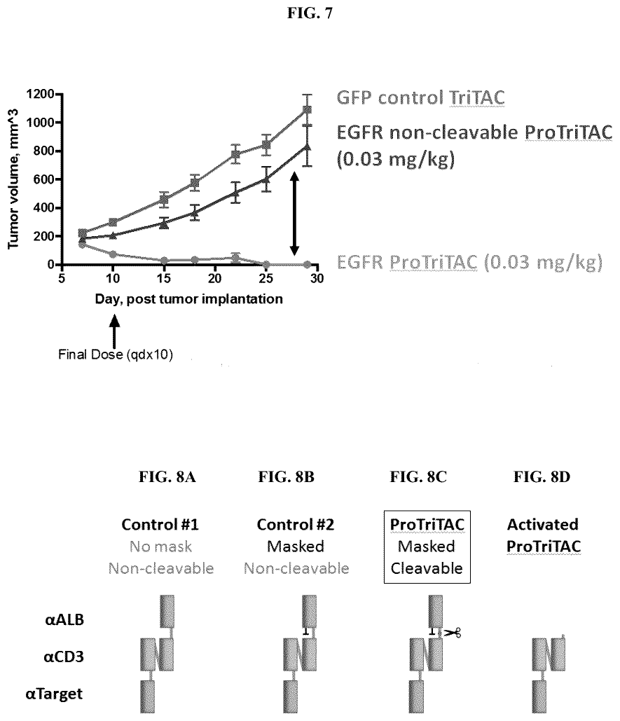

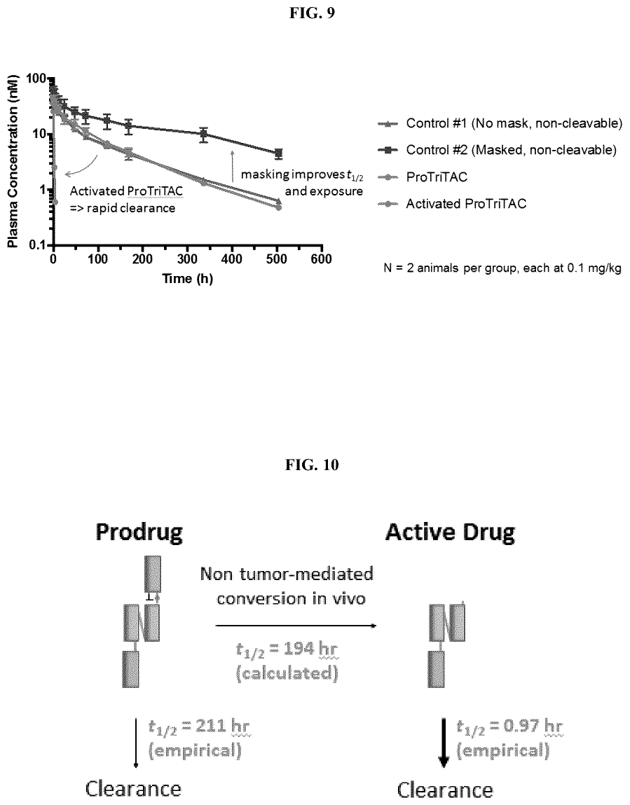

Single doses of PSMA targeting ProTriTAC (SEQ ID NO: 43) containing a PSMA binding domain as the target binding domain, a CD3 binding domain, and an albumin binding domain comprising a masking moiety (SEQ ID NO: 50) and a cleavable linker (SEQ ID NO: 53), non-cleavable PSMA targeting ProTriTAC (SEQ ID NO: 44), non-masked/non-cleavable TriTAC (SEQ ID NO: 52), and active drug mimicking protease-activated PSMA targeting ProTriTAC (SEQ ID NO: 45) were dosed into cynomolgus monkeys at 0.1 mg/kg via intravenous injection. Plasma samples were collected at the time points indicated in FIG. 9. The designs of the above described test molecules are shown in FIG. 8. Concentrations of the various test molecules, as described above, were determined using ligand binding assays with biotinylated recombinant human PSMA (R&D systems) and sulfo-tagged anti-CD3 idiotype antibody cloned 11D3 in a MSD assay (Meso Scale Diagnostic, LLC). Pharmacokinetic parameters were estimated using Phoenix WinNonlin pharmacokinetic software using a non-compartmental approach consistent with the intravenous bolus route of administration.

To calculate the rate of in vivo conversion of the test molecules (i.e., conversion of PSMA targeting ProTriTAC, non-cleavable PSMA targeting ProTriTAC, non-masked/non-cleavable PSMA targeting ProTriTAC) the concentration of active drug in circulation was estimated by solving the following system of differential equations where P is the concentration of prodrug, A is the concentration of active drug, kₐ is the rate of prodrug activation in circulation, k_{c,P} is the clearance rate of the prodrug, and k_{c,A} is the clearance rate of the active drug.

$$\frac{dP}{dt} = - {k}_{c , P} P \\ \frac{dA}{dt} = {k}_{a} P - {k}_{c , A} A$$

The clearance rates of the prodrug, active drug, non-masked non-cleavable prodrug control, and a non-cleavable prodrug control (k_{c,NCLV}) were determined empirically in cynomolgus monkeys. To estimate the rate of prodrug activation in circulation, it was assumed that the difference between the clearance rate of cleavable prodrug and the non-cleavable prodrug arose solely from non-specific activation in circulation. Therefore, the rate of prodrug conversion to active drug in circulation was estimated by subtracting the clearance rate of the cleavable prodrug from the non-cleavable prodrug.

$${k}_{a} = {k}_{c , NCLV} - {k}_{c , P}$$

The initial concentration of prodrug in circulation was determined empirically and the initial concentration of active drug was assumed to be zero. Further calculations showed that the ProTriTAC comprising the protease cleavable linker was sufficiently stable in circulation, with 50% non-tumor mediated conversion every 194 hours and the t_{1/2} of the molecule was determined, empirically, to be around 211 hours. This indicated that ProTriTAC molecules are sufficiently stable and protected against off-tumor effects. In contrast, the t_{1/2} of the active drug fragment mimicking the activated ProTRITAC molecule was determined, empirically, to be 0.97 hours. Thus, active drug was rapidly cleared from circulation. Results are shown in FIG. 10.

FIG. 11 shows that because the active drug mimicking the activated ProTriTAC molecule was rapidly cleared from circulation and that the ProTriTAC and the control non-masked non-cleavable ProTriTAC had longer half-lives, there is a significant >200-fold differential between the ProTriTAC and the activated ProTriTAC circulating exposure. It was also observed that the masked but non-cleavable ProTriTAC control molecule was present in circulation for a longer time than the non-masked non-cleavable ProTriTAC control, thereby indicating that masking plays a role in increased circulation half-life (t_{1/2}) and limited peripheral T cell binding. The combination of the functional masking that renders ProTriTAC inert outside the tumor environment and the half-life differential that renders any aberrantly activated ProTriTAC in circulation to be rapidly cleared in vivo works together to ensure on-target, off-tumor activity of ProTriTAC is minimized. Supporting pharmacokinetic parameters are shown in Table 5.

TABLE 5
Pharmacokinetics of ProTriTAC, Activated
ProTriTAC and Control Molecules

| | Terminal | Cₘₐₓ | AUC, 0-last | Clearance |
|---|---|---|---|---|
| Test Article | t_{1/2} (hr) | (nM) | (hr*nM) | (mL/hr/kg) |
| Control #1 (No mask, | 118 | 48.2 | 2490 | 0.735 |
| non-cleavable) | | | | |
| Control #2 (Masked, | 211 | 58.0 | 7000 | 0.238 |
| non-cleavable) | | | | |
| ProTriTAC | 101 | 42.7 | 2670 | 0.686 |
| Activated ProTriTAC | 0.969 | 66.6 | 41.5 | 58.5 |

### D) Protease Activation of ProTriTAC Molecule Leads to Significantly Enhanced Activity In Vitro

The aim of this study was to assess the relative potency of protease activatable ProTriTAC molecules, non-cleavable ProTriTAC molecules and recombinant active drug fragment mimicking the protease-activated ProTriTAC molecule, in CD3 binding and T cell mediated cell killing. The active drug fragment mimicking the protease activated ProTriTAC molecule contained the CD3 binding domain and the target antigen binding domain but lacked the albumin binding domain. Whereas the protease activatable ProTriTAC molecule contained the albumin binding domain comprising a masking domain and a protease cleavable site, the CD3 binding domain, and the target antigen binding domain. The non-cleavable ProTriTAC molecule lacked the protease cleavable site but was otherwise identical to the protease activatable ProTriTAC molecule.

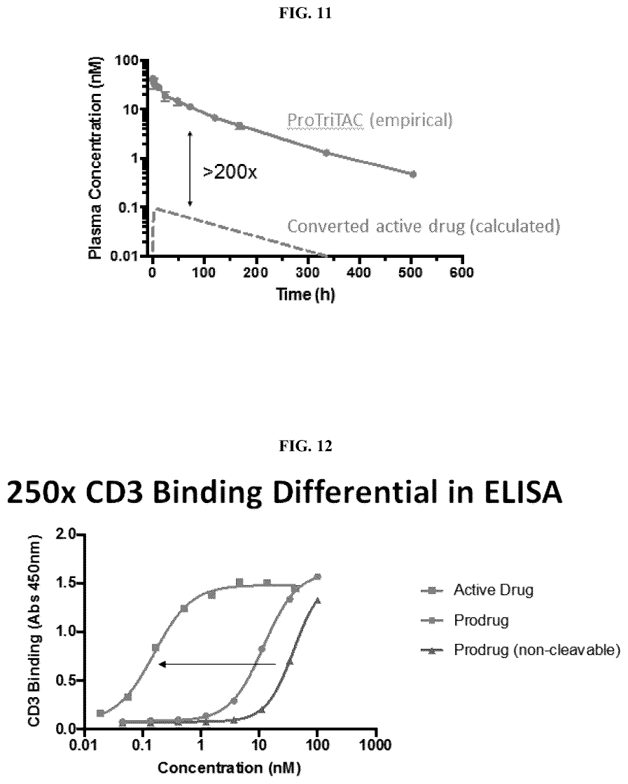

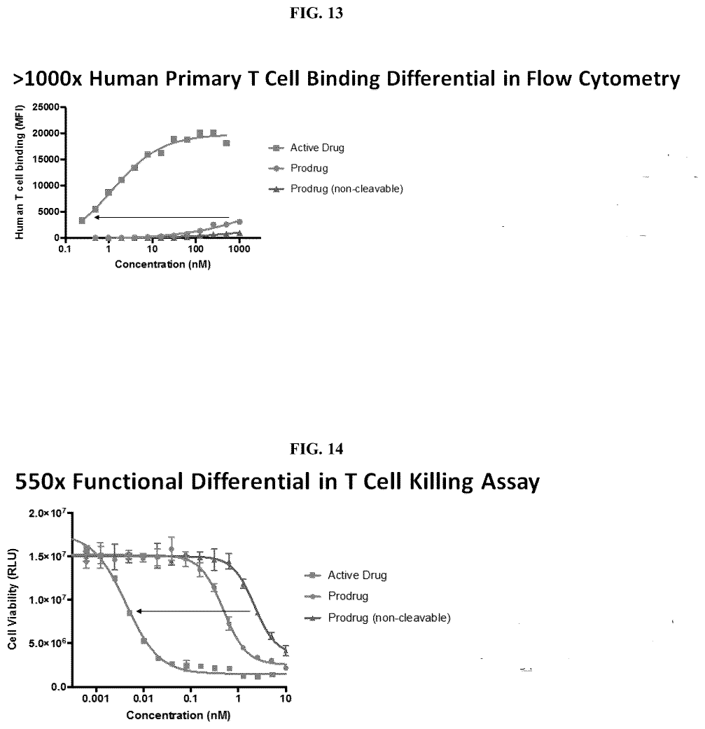

Purified ProTriTAC (labeled as prodrug in FIGS. 12-14), non-cleavable ProTriTAC (labeled as prodrug (non-cleavable) in FIGS. 12-14), and recombinant active drug fragment mimicking the protease-activated ProTriTAC (labeled as active drug in FIGS. 12-14) were tested for binding to recombinant human CD3 in an ELISA assay (FIG. 12), binding to purified human primary T cells in a flow cytometry assay (FIG. 13), and functional potency in a T cell-dependent cellular cytotoxicity assay (FIG. 14).

For ELISA, soluble test molecules (i.e., active drug, prodrug, and prodrug (non-cleavable) at the indicated concentrations were incubated, in multi-well plates, with immobilized recombinant human CD3ε (R&D Systems) for 1 hour at room temperature in PBS supplemented with 15 mg/mL human serum albumin. Plates were blocked using SuperBlock (Thermo Fisher), washed using PBS with 0.05% Tween-20, and detected using a non-competitive anti-CD3 idiotype monoclonal antibody 11D3 followed by peroxidase-labeled secondary antibody and TMB-ELISA substrate solution (Thermo Fisher). Results shown in FIG. 12 demonstrate that the active drug fragment mimicking the protease-activated ProTriTAC molecule was about 250 times more potent in binding CD3 as compared to prodrug non-cleavable. The EC₅₀ values are provided in Table 6. The masking ratio is the ratio between the prodrug EC₅₀ over the active drug EC₅₀: the higher the number, the higher the fold-shift between prodrug and active drug and thus greater functional masking.

TABLE 6
CD3 binding potential

| | EC₅₀ (nM) | Masking Ratio |
|---|---|---|
| Active Drug | 0.16 | — |
| Prodrug | 11.91 | 74 |
| Prodrug (non-cleavable) | 39.44 | 247 |

For binding to human primary T cells, determined by flow cytometry, soluble test molecules (i.e., active drug, prodrug, and prodrug (non-cleavable)) at the indicated concentrations (shown in FIG. 13) were incubated, in multi-well plates, with purified human primary T cells for 1 h at 4° C. in the presence of PBS with 2% fetal bovine serum and 15 mg/ml human serum albumin. Plates were washed with PBS with 2% fetal bovine serum, detected using AlexaFluor 647-labeled non-competitive anti-CD3 idiotype monoclonal antibody 11D3, and data was analyzed using FlowJo 10 (FlowJo, LLC). Results shown in FIG. 13 demonstrate that the active drug fragment mimicking the protease-activated ProTriTAC molecule was greater than 1000 times more potent in binding human primary T cells as compared to prodrug non-cleavable. The EC₅₀ values are provided in Table 7.

TABLE 7
Human primary T cell binding potential

| | EC₅₀ (nM) | Masking Ratio |
|---|---|---|
| Active Drug | 1.19 | — |
| Prodrug | >1000 | n/a |
| Prodrug (non-cleavable) | >1000 | n/a |

For functional potency in a T cell-dependent cellular cytotoxicity assays, soluble test molecules (i.e., active drug, prodrug, and prodrug (non-cleavable)) at the indicated concentrations, shown in FIG. 14, were incubated, in multi-well plates, with purified resting human T cells (effector cell) and HCT116 cancer cell (target cell) at 10:1 effector: target cell ratio for 48 h at 37° C. The HCT116 target cell line had been stably transfected with a luciferase reporter gene to allow specific T cell-mediated cell killing measurement by ONE-Glo (Promega). Results shown in FIG. 14 demonstrate that the active drug fragment mimicking the protease-activated ProTriTAC molecule was about 500 times more potent in T cell mediated killing of cancer cells, as compared to prodrug non-cleavable. The EC₅₀ values are provided in Table 8.

TABLE 8
T cell mediated cell killing potential

| | EC₅₀ (nM) | Masking Ratio |
|---|---|---|
| Active Drug | 0.004 | — |
| Prodrug | 0.485 | 121 |
| Prodrug (non-cleavable) | 2.197 | 549 |

### Example 7: Anti-Tumor Activity of Exemplary ProTriTAC Molecules Containing Various Exemplary Linkers, in an Admixed Mouse Tumor Model

The aim of this study was to explore the anti-tumor activity of ProTriTAC molecules containing different linkers. NSG female mice, 7 weeks old, were used for this study. At the commencement of the study, on day 0, the NSG female mice were injected with 2.5×10⁶ expanded human T cells, and 5×10⁶ HCT116 (human colorectal carcinoma) tumor cells. The following day, on day 1, the mice were divided into groups and each group was treated with at least one of the ProTriTAC molecules listed in Table 9 (SEQ ID Nos. 786-790), or with a control GFP TriTAC molecule (SEQ ID No. 792), or with a ProTriTAC molecule that contains a non cleavable linker (NCLV) (SEQ ID No. 791).

The ProTriTAC molecules and the ProTriTAC NCLV molecule used in the following examples were targeted to EGFR and had the following orientation of the individual domains: (anti-albumin binding domain (sdAb): anti-CD3 domain (scFV): anti-EGFR domain (sdAb)). The only differences between the ProTriTAC molecules listed in Table 6 were in the linker sequences. The ProTriTAC molecules, ProTriTAC NCLV molecule, or the GFP TriTAC molecule (the GFP TriTAC molecule had the following orientation of individual domains: anti-GFP sdAb: anti-Alb sdAb: anti-CD3 scFv) were administered daily for a period of 10 days (i.e., final dose was administered on day 10 following injection of tumor cells and expanded cells to the animals) and tumor volumes were measured at regular intervals, beginning a few days prior to the administration of the final dose at day 10.

TABLE 9
ProTriTAC sequences and linkers

| | Linker in the | | Cleavability/Recognition of |
|---|---|---|---|
| ProTriTAC | ProTriTAC | Linker | the linker sequence by |
| molecule sequence | molecule | Sequence | enzymes |
| SEQ ID No. 786 | L001 | KPLGLQARVV | MMP9 + matriptase |
| SEQ ID No. 787 | L040 | PQASTGRSGG | MMP9 + matriptase + uPA |
| SEQ ID No. 788 | L041 | PQGSTGRAAG | MMP9 + matriptase + uPA |
| SEQ ID No. 789 | L042 | PPASSGRAGG | matriptase + uPA |
| SEQ ID No. 790 | L045 | PIPVQGRAH | MMP9 + matriptase |
| SEQ ID No. 909 | L043 | PQGSTARSAG | MMP9 + matriptase |

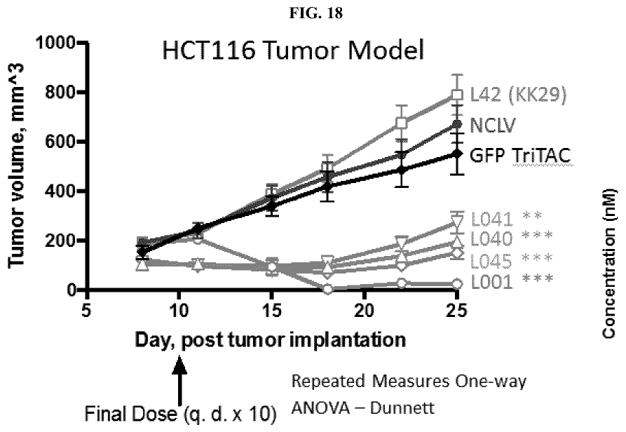

As shown in FIG. 18, the ProTriTAC molecules containing linker sequences L001, L045, L040, and L041 demonstrated more potent anti-tumor activity as compared to the GFP TriTAC control of the ProTriTAC NCLV molecule. The statistical significance of the data was determined by repeated measures one-way ANOVA-Dunnett post-test. The mean tumor volume of each group of mice were compared to the mean tumor volume of the mice group that received the NCLV molecule.

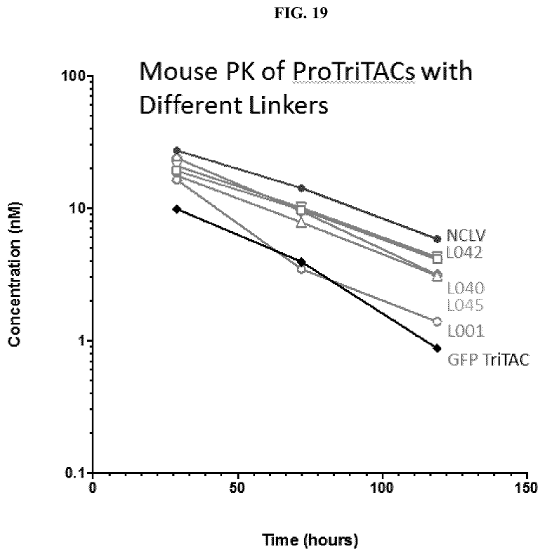

The pharmacokinetics following administration of the various molecules, as described above, were also assessed and the data is shown in FIG. 19. The control GFP TriTAC molecule was rapidly cleared from circulation, following administration, whereas the NCLV molecule remained in circulation for the longest time. The pharmacokinetic clearance profile of the test ProTriTAC molecules were in between the GFP TriTAC and NCLV, except for the ProTriTAC containing the linker sequence L001, which was cleared almost as rapidly as the control GFP TriTAC.

### Example 8: Individual Tumor Volumes of Admixed Xenograft Tumors, Following Treatment with Exemplary ProTriTAC Molecules Containing Various Exemplary Linkers

The ProTriTAC molecules listed in Table 9, the control GFP TriTAC molecule, and the ProTriTAC NCLV molecule were evaluated in an admixed xenograft model, in order to determine the efficacy of the ProTriTAC molecules containing different linkers, in vivo. As described in previous example (Example 7), the xenograft tumor model was generated by injecting 7-week-old NSG mice with 2.5×10⁶ expanded human T cells, and 5×10⁶ HCT116 (human colorectal carcinoma) tumor cells. The mice were divided into groups and each group was treated with at least one of the ProTriTAC molecules listed in Table 9, with the control GFP TriTAC molecule, or with the ProTriTAC NCLV molecule. Tumor volumes were measured at regular intervals, starting from day 10 post injection of tumor cells and expanded T cells.

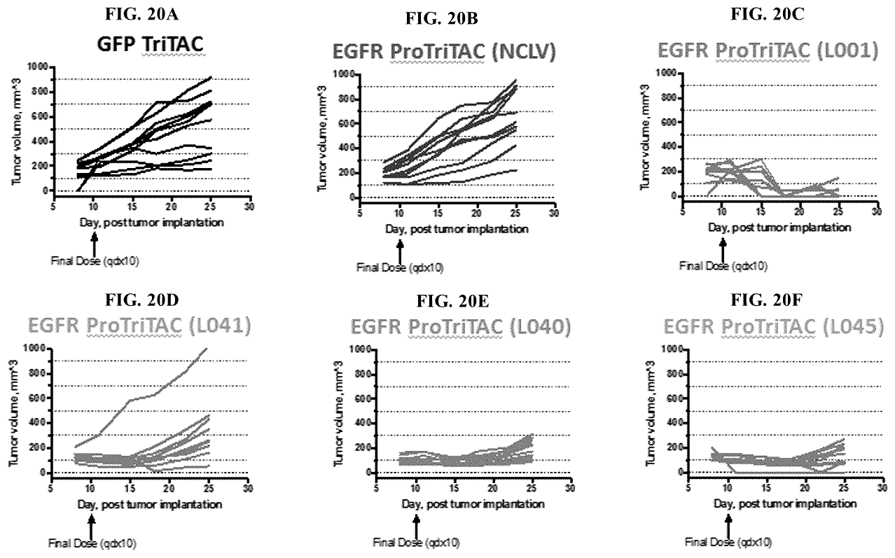

It was observed that in animals treated with the exemplary ProTriTAC molecules containing linker L040 there was a statistically significant delay in tumor growth as compared to the mice group which was treated with the control GFP TriTAC molecule, or the mice group that was treated with the ProTriTAC NCLV molecule. Similar observation was also made for the ProTriTAC molecules containing linker sequences L001, L041, and L045. The data is shown in FIG. 20.

It is also possible to carry out a similar study with xenograft models using other cell lines, such as A549 (non-small cell lung carcinoma) cells, DU-145 (prostate) cells, MCF-7 (breast) cells, Colo 205 (colon) cells, 3T3/]GF-IR (mouse fibroblast) cells, NCI H441 cells, HEP G2 (hepatoma) cells, MDA MB 231 (breast) cells, HT-29 (colon) cells, MDA-MB-435s (breast) cells, U266 cells, SH-SYSY cells, Sk-Mel-2 cells, NCI-H929, RPM18226, and A431 cells.

### Example 9: Demonstration of Reduced Cytokine Levels in Cynomolgus Monkeys, Correlated with Masking of TriTAC Molecules

In this study, cynomolgus monkeys were treated with three different concentrations (30 μg/kg; 300 μg/kg; and 1000 μg/kg) of an exemplary EGFR targeting ProTriTAC molecule containing a non-cleavable linker (ProTriTAC (NCLV), or with three different concentrations (10 μg/kg; 30 μg/kg; and 100 μg/kg) of an exemplary EGFR targeting TriTAC molecule (SEQ ID No. 793).

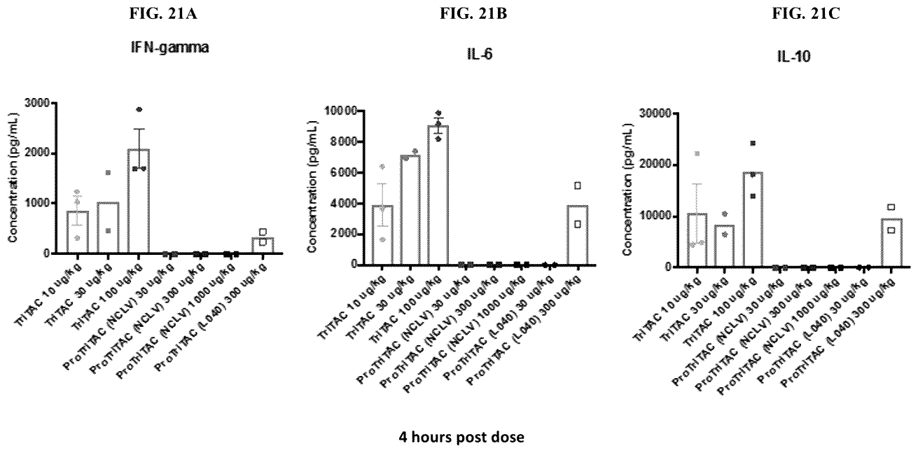

As shown in FIG. 21, after 4 hours following administering the ProTriTAC (NCLV) molecule, IFN-gamma IL-6 and IL-10 levels were significantly lower in comparison with administering the EGFR targeting TriTAC molecule.

### Example 10: Demonstration of Improved Tolerability in Mouse, Conferred by an Exemplary EGFR Targeting ProTriTAC Molecule

In this study, the tolerability of an exemplary EGFR targeting ProTriTAC molecule was assessed. Seven weeks old NSG female tumor free mice were intraperitoneally injected with 2×10⁷ expanded human T cells at the commencement of the study, i.e., at day 0. On day 2, treatment was started by dividing the mice into various groups and administering to them varying concentrations of the exemplary EGFR targeting ProTriTAC molecule, containing the linker sequence L001, an EGFR targeting TriTAC molecule, and an EGFR targeting ProTriTAC molecule containing a non-cleavable linker (ProTriTAC (NCLV). The molecules were administered once daily for 10 days, at the following dosages: 30 μg/kg, 100 μg/kg, 300 μg/kg. Starting from day 2, body weight of the animals was recorded daily.

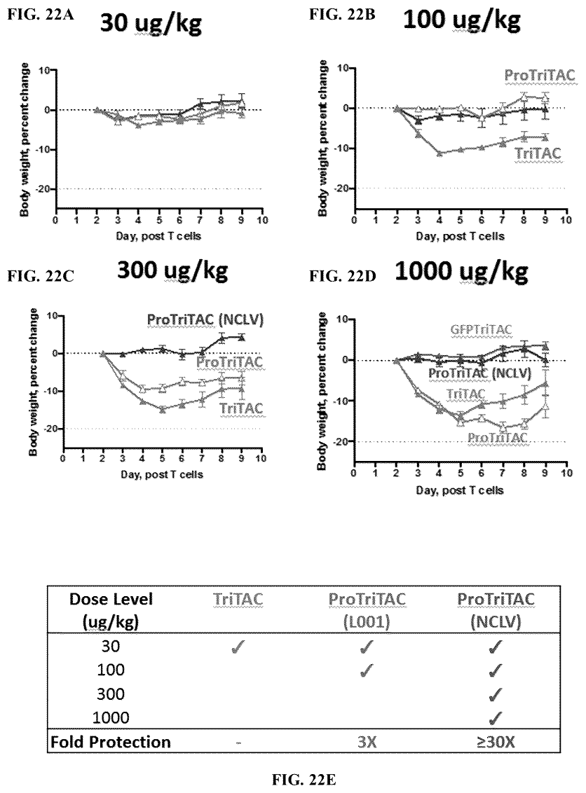

As shown in FIG. 22, the EGFR targeting ProTriTAC molecule containing a non-cleavable linker (ProTriTAC (NCLV)) and a GFP TriTAC (used as a negative control) were very well tolerated in mice even at the highest dose of 1000 μg/kg. The EGFR targeted ProTriTAC molecule containing the linker sequence of L001 was well tolerated at the dosage of 100 μg/kg, whereas the EGFR targeted TriTAC was well tolerated at 30 μg/kg. It was thus observed that the ProTriTAC containing the L001 linker sequence conferred about 3-fold increase in tolerability and the ProTriTAC (NCLV) conferred about a 30 fold increase in tolerability, in mouse. The mouse maximum tolerated dose for the ProTriTAC (NCLV) and TriTAC molecules was consistent with what was observed in cynomolgus monkeys.

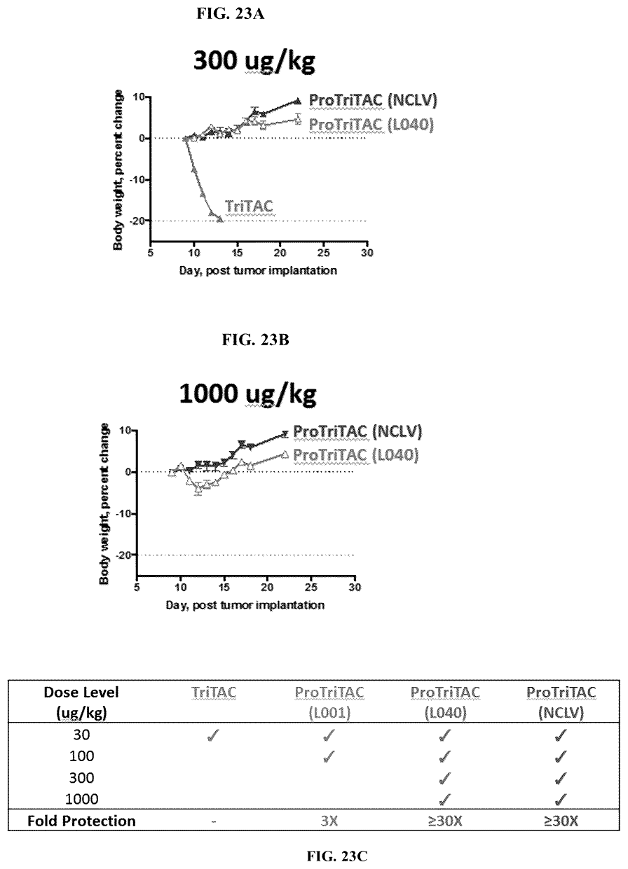

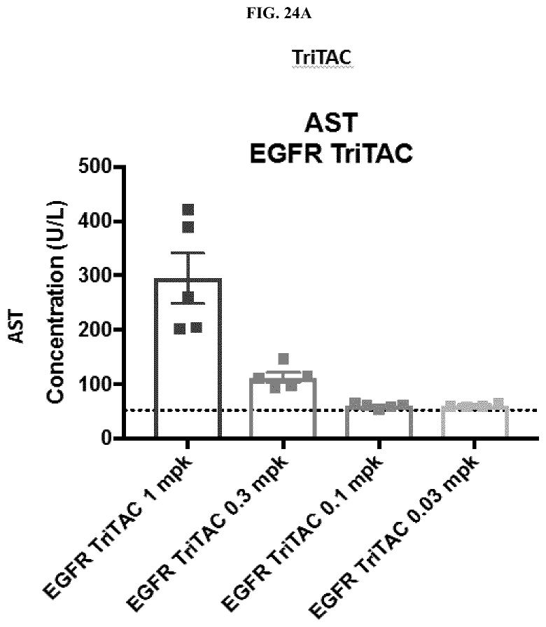

To further explore the role of the linker in tolerability of the EGFR targeting ProTriTAC molecule in mouse, the linker sequence was changed from L001 to that of L040. In this experiment, seven weeks old NSG female tumor free mice were subcutaneously injected with 5×10⁶ HCT116 tumor cells, at the commencement of the study, i.e., at day 0. At day 7 following the tumor cell injection, when the tumor volumes were about 180-200 mm³ (e.g., 183 mm³), the mice were injected intraperitoneally with 2×10⁷ expanded human T cells. Treatment was started on day 9, by dividing the mice into various groups and each group was administered an EGFR targeting TriTAC molecule, an EGFR targeting ProTriTAC molecule with linker sequence L040 (ProTriTAC(L040), and a ProTriTAC molecule containing a non-cleavable linker (ProTriTAC(NCLV). The molecules were administered once daily for 10 days, at the following dosages: 300 μg/kg and 1000 μg/kg. Starting from day 2, body weight of the animals was recorded daily. The results shown in FIG. 23 provide that the EGFR targeting ProTriTAC molecule with the linker sequence L040 conferred better tolerability than when the linker sequence L001 was used. The ProTriTAC (L040) was well-tolerated at 300 μg/kg and at 1000 μg/kg, with body weight percentage change comparable to that with the ProTriTAC (NCLV) molecule. It was thus observed that compared to the TriTAC molecule, the ProTriTAC containing the L040 linker sequence conferred about a 30 fold increase in tolerability, in mouse, similar to the 30 fold increase in tolerability observed with the ProTriTAC (NCLV) molecule.

### Example 11: Clinical Pathology Studies in Mice and Cynomolgus Monkeys

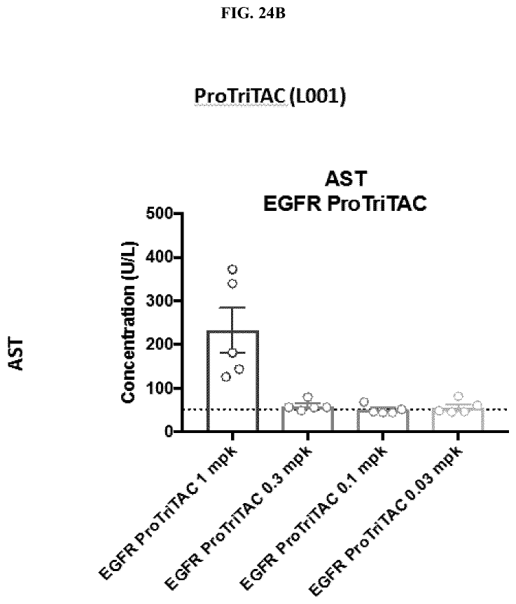

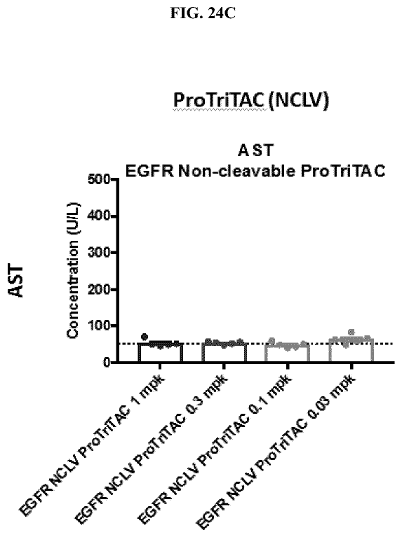

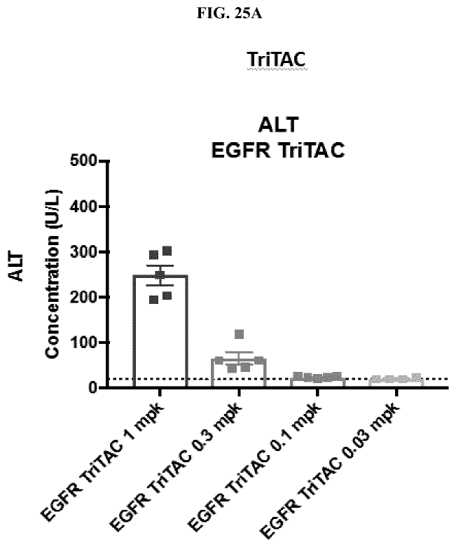

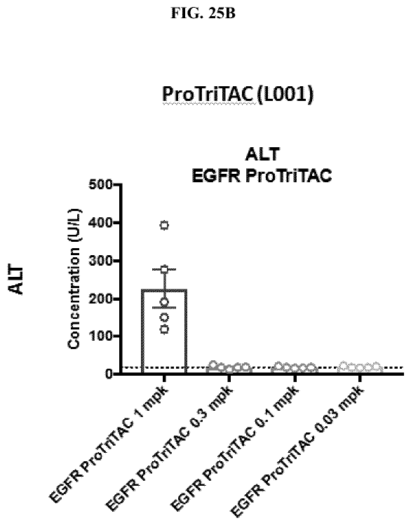

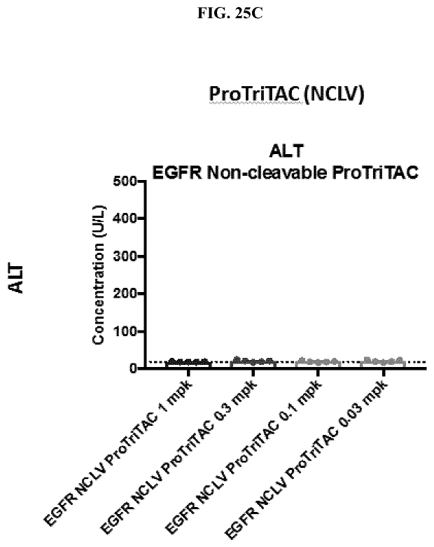

In this study, mice were treated with various concentrations of an EFGR targeting TriTAC molecule, an EGFR targeting ProTriTAC molecule containing the linker sequence L001 (ProTriTAC (L001), and an EGFR targeting ProTriTAC molecule containing a non-cleavable linker (ProTriTAC(NCLV)). Tolerability was assessed by measuring serum concentration of ALT (alanine aminotransferase) and AST (aspartate aminotransferase). Results are shown in FIGS. 24A, 24B, and 24C and FIGS. 25A, 25B, and 25C. It was observed that serum concentrations of AST and ALT were not elevated following administering the ProTriTAC (L001) at dosages up to 0.3 mg/kg, and that the serum concentrations of AST and ALT were not elevated following administering the ProTriTAC(NCLV) at dosages up to 1 mg/kg. In contrast, serum concentration of AST and ALT were not elevated following administering the TriTAC molecule at a dosage of 0.3 mg/kg.

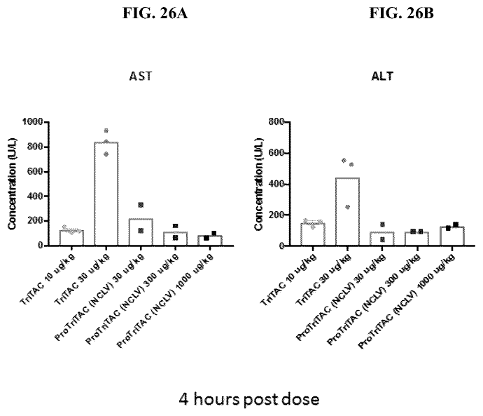

In another study, cynomolgus monkeys were treated with various concentrations of an EFGR targeting TriTAC molecule, and an EGFR targeting ProTriTAC molecule containing a non-cleavable linker (ProTriTAC(NCLV)). Tolerability was assessed by measuring serum concentration of ALT (alanine aminotransferase) and AST (aspartate aminotransferase). Results are shown in FIG. 26. It was observed that serum concentrations of AST and ALT were not elevated following administering the ProTriTAC (NCLV) at dosages up to 1000 μg/kg. In contrast, serum concentration of AST and ALT were not elevated following administering the TriTAC molecule at a dosage of 10 μg/kg.

### Example 12: Demonstration of Therapeutic Window Expansion with an Exemplary ProTriTAC Molecule of this Disclosure in a Tumor-Bearing Mouse Model

The aim of this study was to evaluate the expansion of therapeutic window by measuring anti-tumor activity and observable on-target toxicity in the same tumor-bearing mice. NSG female mice, 7 weeks old, were used for this study.

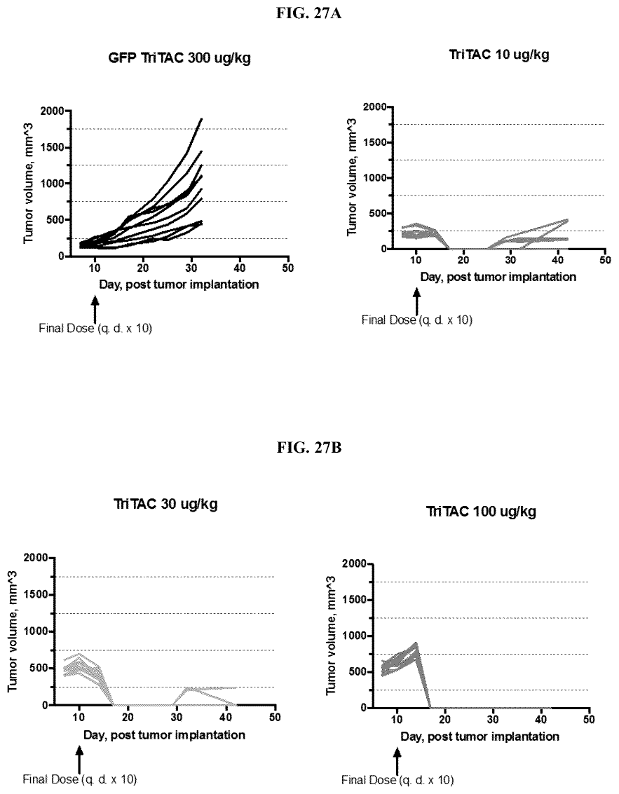

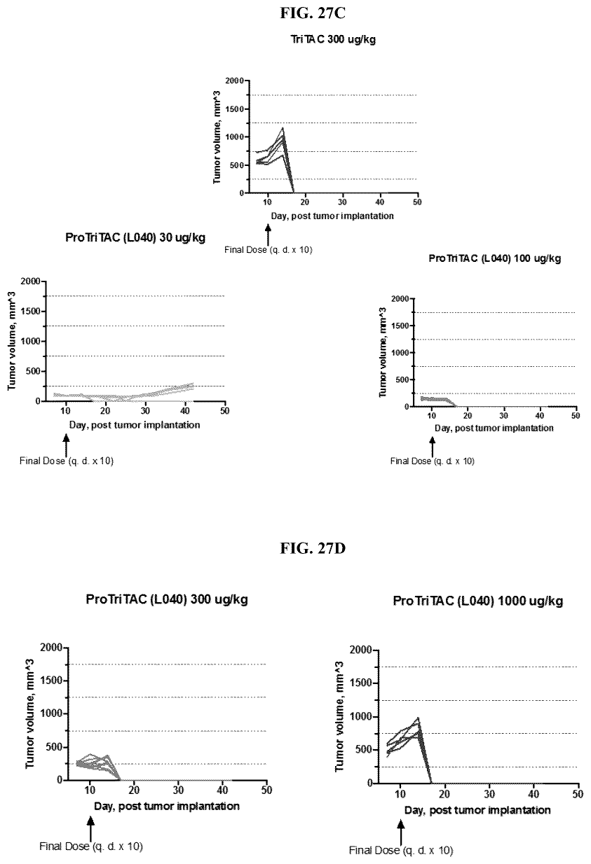

At the commencement of the study, on day 0, the NSG female mice were injected with 2.5×10⁶ expanded human T cells, and 5×10⁶ HCT116 (human colorectal carcinoma) tumor cells. The following day, on day 1, the mice were divided into groups and each group was treated with either GFP TriTAC molecule (SEQ ID No. 792), EGFR TriTAC molecule (SEQ ID No. 793), or an EGFR targeting ProTriTAC molecule containing linker L040, (SEQ ID No. 787) at the indicated dose levels in FIGS. 27A-297D (for GFP TriTAC the dosage was 300 μg/kg; for EGFR TriTAC dosages were 10 μg/kg, 30 μg/kg, 100 μg/kg, and 300 μg/kg; for EGFR ProTriTAC dosages were 30 μg/kg, 100 μg/kg, 300 μg/kg, and 1000 μg/kg) and administered daily for a period of 10 days (i.e., final dose was administered on day 10 following injection of tumor cells and expanded cells to the animals) and tumor volumes were measured at regular intervals, beginning a few days prior to the administration of the final dose at day 10. Results shown in FIGS. 27A-27D.

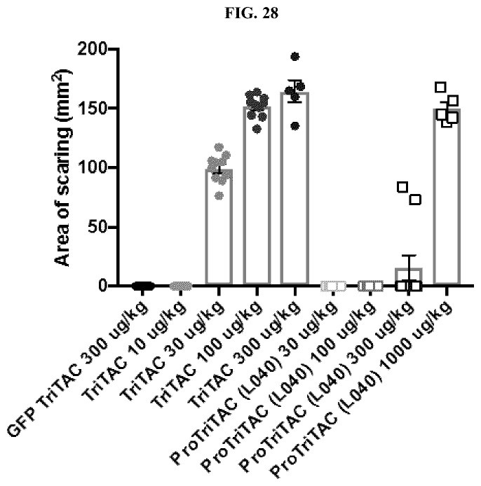

On-target EGFR-related toxicity was determined by measuring the radius of the red scarring skin lesion above the original tumor implantation site with a caliper and applying the equation Area=π*(radius oflesion)² on day 14. Results provided in FIG. 28 show that an exemplary EGFR ProTriTAC has 30× better tolerability compared to an EGFR TriTAC in the same HCT116 tumor-bearing mice as measured by the onset of red scarring skin lesions above the original tumor implantation site. Therapeutic window is defined as the difference between the minimal dose level required for anti-tumor activity and the highest skin lesion-free dose level.

Results (from FIGS. 27 and 28) show that the exemplary protease-cleavable EGFR ProTriTAC is 3× less potent but 30× more tolerated (i.e., has a 10× improved therapeutic window) than the EGFR TriTAC when efficacy and toxicity are measured on the same tumor-bearing mice, as summarized in below Table. This would make it possible to dose the ProTriTAC at a dose that is about 3× higher than the TriTAC, to get at least the same efficiency and better tolerability.

| | Min efficacious | Max scar- | Therapeutic |
|---|---|---|---|
| | dose | free dose | Window |
| TriTAC | 10 μg/kg | 10 μg/kg | 1 |
| ProTriTAC | 30 μg/kg | 300 μg/kg | 10 |

### Example 13: An Exemplary ProTriTAC Molecule Containing a Binding Moiety with Extended Non-CDR Loop into which a Human CD3ε Epitope is 2Rafted

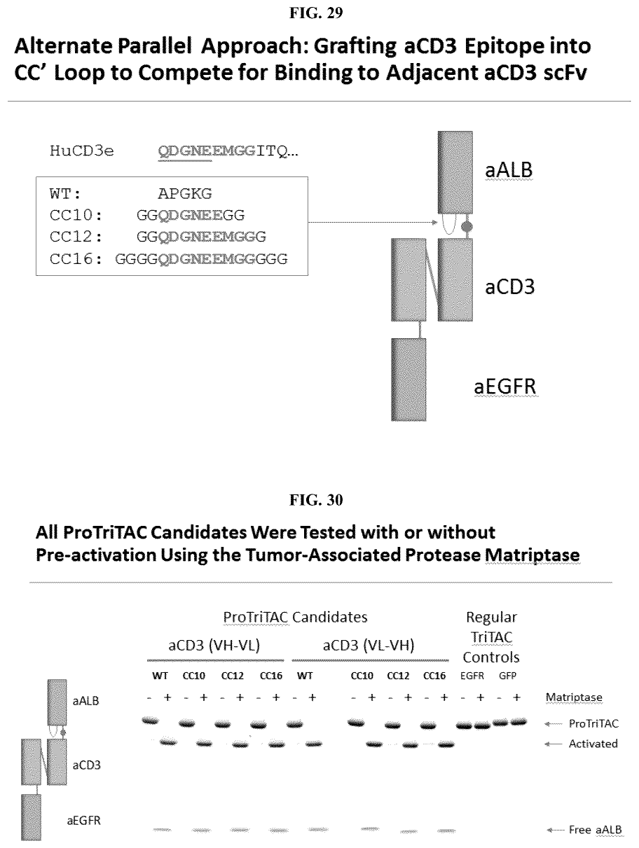

The sequence of a binding moiety comprising non-CDR loops (AB, EF, C″D, and CC′) was obtained. A portion of the human CD3ε sequence was grafted into the CC′ loop of the non-CDR loops within the binding moiety, along with glycine residues to further extend the CC′ loop. FIG. 29 illustrates three different variants comprising 10, 12, or 16 amino acid extensions to the CC′ loop. In case of the variant CC10, the portion of human CD3ε sequence grafted into the CC′ loop, to replace the wild type sequence of APGKG was QDGNEE (SEQ ID NO. 801), and in addition 4 glycine residues were inserted to extend the CC′ loop. In case of the variant CC12, the portion of human CD3ε sequence grafted into the CC′ loop, to replace the wild type sequence of APGKG was QDGNEEMGG (SEQ ID No. 802), and in addition 3 glycine residues were inserted to extend the CC′ loop. In case of the variant CC12, the portion of human CD3E sequence grafted into the CC′ loop, to replace the wild type sequence of APGKG was QDGNEEMGG (SEQ ID No. 803), and in addition 7 glycine residues were inserted to extend the CC′ loop. The binding moiety comprising extended non-CDR loops comprising the CD3E sequences, as described above, were cloned into a vector further comprising coding sequences for a protease cleavable linker, a scFv containing a CD3 binding domain, and an EGFR binding domain, to express a ProTriTAC molecule. The ProTriTAC molecule contained an exemplary binding moiety of this disclosure, a CD3 binding scFv, and an EGFR binding domain. The ProTriTAC molecule was subsequently exposed to a tumor associated protease, matriptase, to assay activation of the molecule upon cleavage of the protease cleavable linker, which separates the binding moiety (depicted as aALB in FIGS. 29 and 30) comprising the cleavable linker from the rest of the molecule, i.e., the scFv containing the CD3 binding domain (depicted as aCD3 in FIGS. 29 and 30) and the EGFR binding domain (aEGFR). FIG. 30 shows the activation of the ProTriTAC molecules, containing the CC10, CC12, or CC16 variants of CC′ non-CDR loop, CD3 binding scFv in a VH-VL (left panel) or a VL-VH (right panel) format, upon treatment with matriptase. A ProTriTAC molecule containing a wild-type CC′ loop in the binding moiety was used as a control for the protease activation assay. In addition, a TriTAC molecule that is not in the “pro” form, i.e., a molecule that includes the same domains as the ProTriTAC molecule, except that it has a half-life extension domain, such as albumin, instead of a binding moiety, was also treated with matriptase and used as a control. Results indicated that the ProTriTAC molecules were activated upon cleavage, to generate a free albumin binding domain (depicted as Free aALB in FIG. 30) whereas the albumin domain did not separate from the TriTAC molecules. Thus, the ProTriTAC molecules containing a binding moiety of this disclosure was able to readily dissociate from half-life extending domain upon cleavage in a tumor microenvironment, unlike the TriTAC versions, and thereby were amenable to rapid clearance from the systemic circulation upon activation.

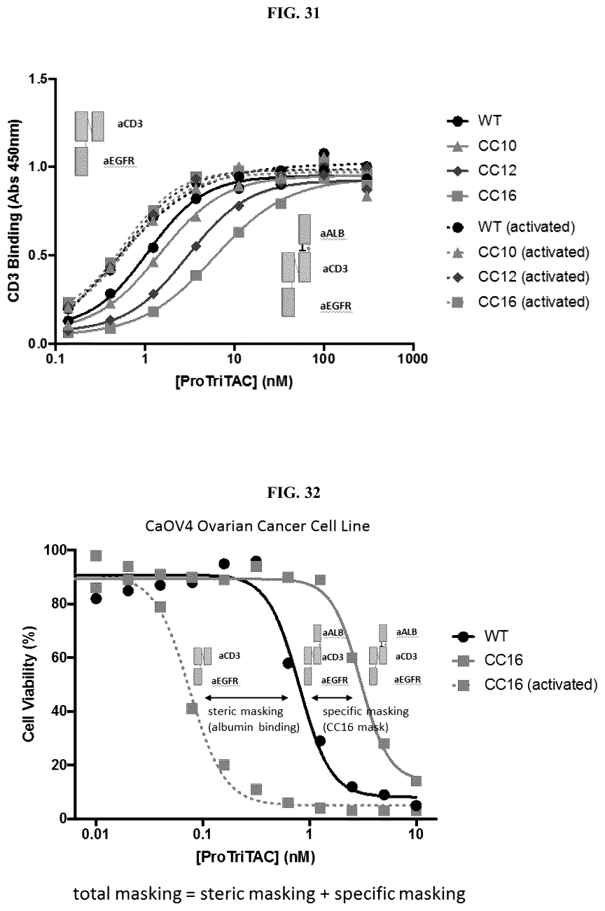

Further studies were carried out to assay the binding of the binding moiety containing the human CD3ε to CD3. It was observed that, in the presence of human serum albumin, the activated forms of ProTriTAC molecules that contained the binding moiety containing the human CD3ε were about 20 times potent in binding CD3 than their activated forms which did not contain the binding moiety. Results are shown in FIG. 31.

Cell killing potential of a ProTriTAC molecule that contained a binding moiety as described herein was also assayed in a study where CaOV4 cell line was treated with the ProTriTAC molecule or its activated form in the presence of human serum albumin. As shown in FIG. 32, the ProTriTAC molecule containing the CC16 variant of CC′ non-CDR loop was about 50 times more potent in killing cancer cells compared to its activated form, which was separated from the albumin binding domain. The total observed masking is a combination of steric masking due to binding to human serum albumin and specific masking from the CC16 mask in the CC′ non-CDR loop.

### Example 14: Soft Library Mutagenesis Identified CC′ Loop as Most Amenable to Modification

To identify locations within the non-CDR loops (AB, CC′, C″D, and EF) that were most amenable to modification, for creating a masking capability, libraries were assembled and generated using four groups of overlapping DNA oligos containing randomized degenerate “NNK” codons and with different loop lengths, as indicated in the schematic below:

### AB Loop Oligos:

WT:
(SEQ ID NO: 903)
LVQPGN (20%)

AB0: XXXXXX (20%)
AB1: XXXXXXX (20%)
AB2: XXXXXXXX (20%)
AB3: XXXXXXXXX (20%)
CC′ loop oligos:
WT: APGKG (20%)
CC0: XXXXX (20%)
CC1: XXXXXX (20%)
CC2: XXXXXXX (20%)
CC3: XXXXXXXX (20%)

### C″D Loop Oligos:

WT:
(SEQ ID NO: 904)
DSVKGR (20%)

CD0: XXXXXX (20%)
CD1: XXXXXXX (20%)
CD2: XXXXXXXX (20%)
CD3: XXXXXXXXX (20%)

### EF Loop Oligos:

WT:
(SEQ ID NO: 905)
SLRPED (20%)

EF0: XXXXXX (20%)
EF1: XXXXXXX (20%)
EF2: XXXXXXXX (20%)
EF3: XXXXXXXXX (20%)

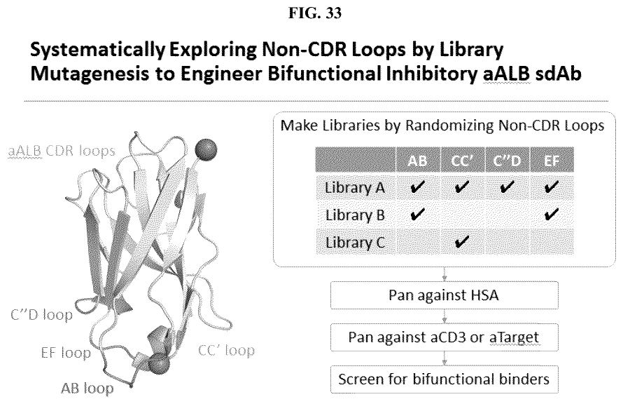

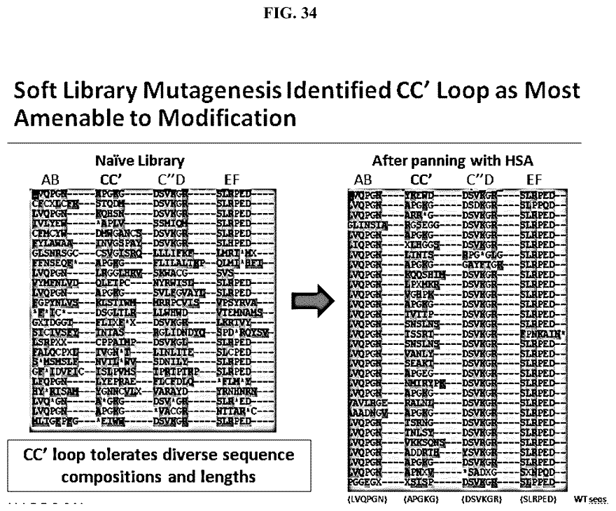

Note: “X” denotes a randomized residue (“NNK” codon) that could be any of the 20 natural amino acids as well as stop codon. The goal was to have approximately 20% of each non-CDR loop be wild-type. These wild-type oligos served as internal benchmarks to gauge the tolerance of each loop to modification (sequence composition and/or length changes). A loop that was less tolerable to change could easily revert to wild-type; in contrast, a loop that was highly amenable to change would maintain the diverse sequence repertoire. To this end, 24 clones were sequenced from the naive library to verify the randomization of non-CDR loops prior to panning with HAS, as shown in FIG. 33. After two rounds of phage panning against HSA, 30 clones were sequenced to examine the non-CDR loop composition. The results showed that 3 out of 4 non-CDR loops (AB, C″D, and EF) mostly utilized the 20% wild-type oligo and reverted back to the wild-type, suggesting that they may be less preferred than the wild-type sequence. However, the CC′ loop kept the diverse sequence repertoire (both sequence and length), suggesting that the CC′ loop may be the loop most tolerable to randomization that we could exploit for specific masking of adjacent domains, as shown in FIG. 34.

### Example 15: Screening of Phage Display Library for Identification of EpCAM Binding Domains

Llamas were immunized with purified EpCAM protein expressed in Expi293 cells. A phage display library for expression of heavy variable antibody domains was constructed from circulating B cells. See van der Linden, de Geus, Stok, Bos,van Wassenaar, Verrips, and Frenken. 2000. J Immunol Methods 240:185-195. Phage clones were screened for binding to EpCAM by expressing anti-EpCAM proteins in E coli, preparing periplasmic extracts, and proteins were screened for human and cynomolgus EpCAM binding activity using a colorimetric ELISA. Thirty-eight unique heavy chain only sequences were identified (SEQ ID Nos. 804-841) that produced a signal in the ELISA screening relative to the control with human and/or cynomolgus EpCAM proteins (as shown in Table 10).

TABLE 10
Binding of Llama anti-Human EpCAM heavy chain only single
domain antibodies to Human and Cynomolgus EpCAM, as
demonstrated by signal in an ELISA Assay (absorbance
readings in a colorimetric ELISA assay), relative to
control heavy chain only single domain antibodies

| | ELISA | ELISA | | Human | Cynomolgus |
|---|---|---|---|---|---|
| Sequence | Human | Cynomolgus | ELISA | EpCAM/ | EpCAM/ |
| name | EpCAM | EpCAM | Control | Control | Control |
| EPL90 | 1.6 | 1.7 | 0.2 | 10 | 10 |
| EPL118 | 3.4 | 2.9 | 0.7 | 5 | 4 |
| EPL138 | 3.1 | 2.7 | 0.9 | 3 | 3 |
| EPL145 | 0.6 | 0.4 | 0.2 | 3 | 2 |
| EPL164 | 0.6 | 2.8 | 0.1 | 7 | 34 |
| EPL31 | 0.6 | 0.5 | 0.1 | 7 | 6 |
| EPL55 | 0.4 | 0.6 | 0.1 | 5 | 7 |
| EPL57 | 1.7 | 3.4 | 0.1 | 14 | 27 |
| EPL136 | 0.9 | 1.3 | 0.1 | 9 | 13 |
| EPL15 | 0.7 | 3.2 | 0.1 | 8 | 35 |
| EPL34 | 0.9 | 3.1 | 0.1 | 10 | 36 |
| EPL86 | 0.8 | 3.0 | 0.1 | 8 | 31 |
| EPL153 | 3.1 | 2.2 | 0.1 | 31 | 21 |
| EPL20 | 2.9 | 2.2 | 0.5 | 6 | 4 |
| EPL70 | 3.2 | 2.2 | 0.1 | 24 | 16 |
| EPL125 | 3.5 | 3.5 | 0.5 | 8 | 8 |
| EPL13 | 2.9 | 4.0 | 0.2 | 17 | 23 |
| EPL129 | 2.0 | 3.1 | 0.1 | 20 | 29 |
| EPL159 | 0.4 | 0.2 | 0.1 | 3 | 2 |
| EPL120 | 3.2 | 2.3 | 0.6 | 5 | 4 |
| EPL126 | 3.4 | 2.8 | 0.7 | 5 | 4 |
| EPL60 | 1.1 | 3.5 | 0.1 | 10 | 33 |
| EPL156 | 3.6 | 4.0 | 0.2 | 16 | 17 |
| EPL2 | 2.2 | 3.6 | 0.1 | 15 | 24 |
| EPL43 | 1.8 | 3.8 | 0.1 | 13 | 27 |
| EPL10 | 2.7 | 2.0 | 0.2 | 12 | 9 |
| EPL49 | 1.1 | 0.6 | 0.1 | 8 | 4 |
| EPL58 | 1.1 | 0.5 | 0.1 | 8 | 4 |
| EPL74 | 1.4 | 0.7 | 0.1 | 10 | 5 |
| EPL78 | 3.1 | 2.0 | 0.2 | 17 | 11 |
| EPL82 | 0.9 | 1.1 | 0.2 | 4 | 5 |
| EPL83 | 2.0 | 1.1 | 0.6 | 3 | 2 |
| EPL97 | 2.5 | 1.7 | 0.3 | 8 | 5 |
| EPL109 | 0.4 | 0.1 | 0.1 | 5 | 2 |
| EPL117 | 0.9 | 0.6 | 0.2 | 4 | 3 |
| EPL127 | 0.4 | 0.9 | 0.1 | 5 | 11 |
| EPL152 | 3.4 | 2.8 | 0.8 | 4 | 4 |
| EPL189 | 1.3 | 0.9 | 0.1 | 12 | 8 |

### Example 16: Incorporation of EpCAM Binding Heavy Chain Only Single Domain Antibodies Into Fusion Proteins and T Cell Dependent Cellular Cytotoxicity Assays

Selected anti-EpCAM heavy chain only single domain antibodies from Example 15 were cloned into DNA constructs for expression of recombinant proteins. These expression constructs all encoded a signal peptide. One set of anti-EpCAM constructs (SEQ ID Nos. 842 to 868) was designed to express a fusion protein with a humanized anti-CD3 scFv domain on the N-terminus of the mature secreted fusion protein followed by a llama anti-EpCAM domain, with the two domains linked by the sequence GGGGSGGGS, and with a HHHHHH on the C-terminus. One second of anti-EpCAM constructs (SEQ ID Nos. 869 to 895) was designed to express a fusion protein with a llama anti-EpCAM domain on the N-terminus of the mature secreted fusion protein followed a humanized anti-CD3-scFv domain, with the two domains linked by the sequence GGGGSGGGS, and with aHHHHHH on the C-terminus.

These anti-EpCAM/anti-CD3 (from N-terminus to C-terminus) or anti-CD3/anti-EpCAM (from N terminus to C terminus) fusion protein constructs were transfected into Expi293 cells. The amount of anti-EpCAM/anti-CD3 fusion protein in the conditioned media from the transfected Expi293 cells was quantitated using by using an Octet instrument with streptavidin and loaded with biotinylated CD3-Fc fusion protein using an anti-CD3 fusion protein of similar molecular weight to the anti-EPCAM/ant-CD3 proteins as a standard.

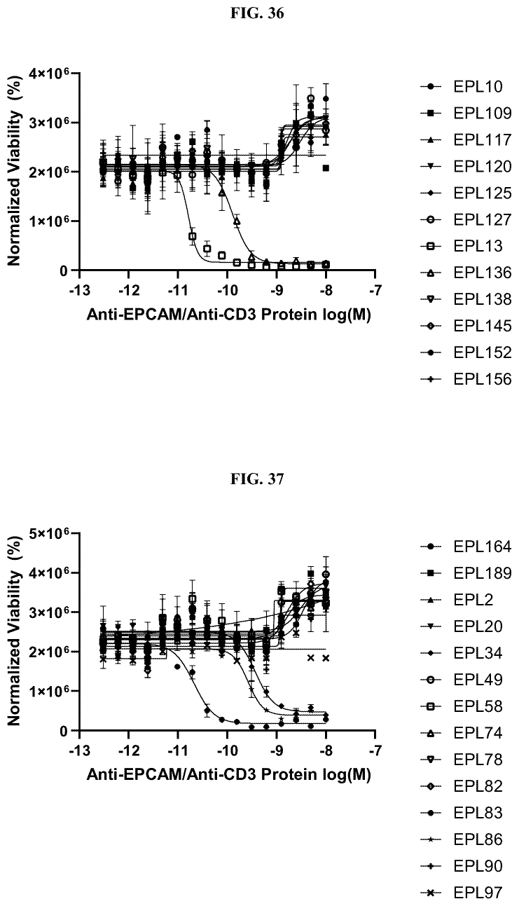

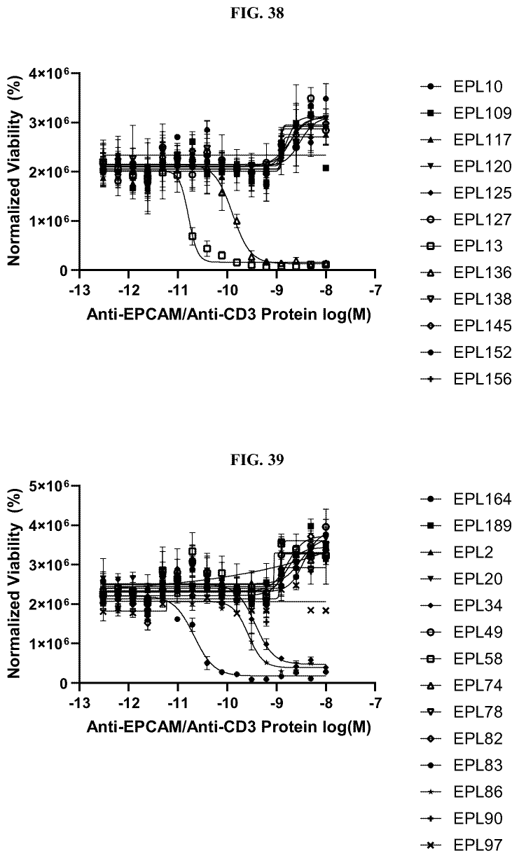

The conditioned media were tested in a T-cell dependent cellular cytotoxicity assay. See Nazarian A A, Archibeque I L, Nguyen Y H, Wang P, Sinclair A M, Powers D A. 2015. J Biomol Screen. 20:519-27. In this assay, luciferase labelled NCI-H508 cells, which express EpCAM, were combined with purified human T cells and a titration of the anti-EpCAM/anti-CD3 fusion protein or the anti-CD3/anti-EpCAM. It was hypothesized that if the fusion protein directs T cells to kill the NCI-H508 cells, the signal in a luciferase assay performed at 48 hours after starting the experiment should decrease. FIGS. 36-39 provide the TDCC data in graphical format. EC₅₀ values from the TDCC assays are listed in Table 11 (lists EC₅₀ data for SEQ ID Nos. 842 to 868) and Table 12 (lists EC₅₀ data for SEQ ID Nos. 869-895). The most potent molecule (EPL13) had an EC₅₀ value of about 1.6 pM. Some of the anti-EpCAM binding proteins were only active when present in an anti-CD3/anti-EpCAM configuration. One anti-EpCAM sequence, EPL34, was only active in the anti-EpCAM/anti-CD3 configuration. A negative control for the TDCC assays was anti-GFP/anti-CD3 protein, and this protein did not direct the T cells to kill the NCI-H508 cells (data not shown).

TABLE 11
EC₅₀ Values for Redirected T Cell Killing of NCI-H508 Cells
by Anti-CD3/Anti-EPCAM Proteins Containing Llama Anti-
EPCAM Sequences (n/a = insufficient activity to calculate
an EC₅₀ using the protein concentrations tested)

| Anti-EpCAM Sequence | NCI-H508 Cell Killing EC₅₀ (M) |
|---|---|
| EPL10 | n/a |
| EPL109 | 2.2E−09 |
| EPL117 | n/a |
| EPL120 | 5.8e−010 |
| EPL125 | 1.3E−09 |
| EPL127 | n/a |
| EPL13 | 1.6E−12 |
| EPL136 | 6.1E−12 |
| EPL138 | 7.9E−12 |
| EPL145 | n/a |
| EPL152 | n/a |
| EPL153 | 1.4E−10 |
| EPL156 | n/a |
| EPL164 | 3.6E−10 |
| EPL189 | n/a |
| EPL2 | n/a |
| EPL20 | n/a |
| EPL34 | n/a |
| EPL49 | n/a |
| EPL58 | n/a |
| EPL74 | 2.6E−09 |
| EPL78 | n/a |
| EPL82 | n/a |
| EPL83 | 3.1E−10 |
| EPL86 | 4.7E−10 |
| EPL90 | 9.2E−12 |
| EPL97 | n/a |

TABLE 12
EC₅₀ Values for Redirected T Cell Killing of NCI-H508 Cells
by Anti-EpCAM/Anti-CD3 Proteins Containing Llama Anti-
EPpCAM Sequences (n/a = insufficient activity to calculate
an EC₅₀ using the protein concentrations tested)

| Anti-EPCAM Sequence | NCI-H508 Cell Killing EC50 (M) |
|---|---|
| EPL10 | n/a |
| EPL109 | n/a |
| EPL117 | n/a |
| EPL120 | n/a |
| EPL125 | n/a |
| EPL127 | n/a |
| EPL13 | 1.6E−11 |
| EPL136 | 1.3E−10 |
| EPL138 | n/a |
| EPL145 | n/a |
| EPL152 | n/a |
| EPL153 | not expressed |
| EPL156 | n/a |
| EPL164 | n/a |
| EPL189 | n/a |
| EPL2 | n/a |
| EPL20 | n/a |
| EPL34 | 3.7E−10 |
| EPL49 | n/a |
| EPL58 | n/a |
| EPL74 | n/a |
| EPL78 | n/a |
| EPL82 | n/a |
| EPL83 | 2.1E−11 |
| EPL86 | 2.7E−10 |
| EPL90 | n/a |
| EPL97 | n/a |

Using conditioned media with known concentrations of anti-EpCAM/anti-CD3 or anti-CD3/anti-EpCAM fusion proteins, the binding affinities of the fusion proteins for human and cynomolgus monkey EpCAM proteins were measured. An Octet instrument with streptavidin tips were loaded with biotinylated human or cynomolgus EpCAM protein, and K_{D} values were calculated by measuring the on rate and off rate of binding of the anti-EPCAM/anti-CD3 fusion or anti-CD3/anti-EpCAM fusion proteins to the biotinylated EpCAM proteins. The K_{D} measurements were made using a single 50 nM concentration of the anti-EPCAM/anti-CD3 or anti-CD3/anti-EpCAM fusion proteins, which allowed for rank ordering potency. The measured relative affinities are listed in Table 13. All of the fusion proteins bound to cynomolgus EpCAM, with K_{D} values ranging from 1.6 to 56 nM. Most, but not all of the fusion proteins were measured binding to human EpCAM with K_{D} values ranging from 0.8 to 74 nM.

TABLE 13
Binding Affinities to Human and Cyno EpCAM of Anti-
EPCAM/Anti-CD3 or Anti-CD3/Anti-EpCAM Fusion Proteins
Containing Llama Anti-EpCAM Sequences

| | Anti-CD3/Anti-EpCAM | | Anti-EpCAM/Anti-CD3 | |
|---|---|---|---|---|
| Humanized anti- | hu KD | cy KD | hu KD | cy KD |
| EpCAM Binder | (nM) | (nM) | (nM) | (nM) |
| EPL13 | 29 | 13 | 14 | 7.6 |
| EPL136 | 74 | 56 | 32 | 31 |
| EPL138 | 2.2 | 2.2 | 0.8 | 1.3 |
| EPL153 | n/q | 3.5 | No expression | |
| EPL164 | 3.1 | 3.6 | 2.1 | 2.9 |
| EPL34 | n/q | 14 | n/q | 6.4 |
| EPL83 | 1.1 | 3 | 1.6 | 3 |
| EPL86 | n/q | 8.3 | n/q | 7 |
| EPL90 | 1.9 | 1.6 | 0.8 | 1.2 |

### Example 17: Humanization of EpCAM Binding Heavy Chain Only Single Domain Antibodies and T Cell Dependent Cellular Cytotoxicity Assays

Three of the llama anti-EpCAM antibodies sequences identified in Example 15 were humanized by grafting their CDR sequences onto human germline sequences, while retaining some llama framework sequences to ensure the antibodies did not lose activity (SEQ ID Nos. 896 to 898).

These sequences were cloned into expression constructs for expression of anti-EpCAM/anti-CD3 fusion proteins (SEQ ID Nos. 899 to 901) in Expi293 cells, as described in Example 16.

The amount of anti-EpCAM/anti-CD3 fusion proteins present in the conditioned medium was quantitated as described in Example 16. The affinities of these humanized proteins for human, cynomolgus, and mouse EpCAM were measured as described in Example 16. The relative K_{D} values calculated from these measurements are listed in Table 14. All three sequences bound to human and cynomolgus EpCAM, with relative K_{D} values ranging from about 0.3 to about 18 nM. Two of the sequences also bound to mouse EpCAM, with K_{D} values ranging from about 1.4 to about 1.8 nM.

TABLE 14
Binding Affinities to Human, Cynomolgus, and Mouse
EpCAM of Anti-EpCAM/Anti-CD3 Fusion Proteins Containing
Llama Anti-EpCAM Sequences (n/q = not quantifiable
under the experimental conditions used)

| Humanized Anti- | Human | Cynomolgus | Mouse |
|---|---|---|---|
| EpCAM Sequence | EpCAM (nM) | EpCAM (nM) | EPCAM (nM) |
| H13 | 17 | 18 | n/q |
| H90 | 0.3 | 1.3 | 1.4 |
| H138 | 0.3 | 1.8 | 1.8 |

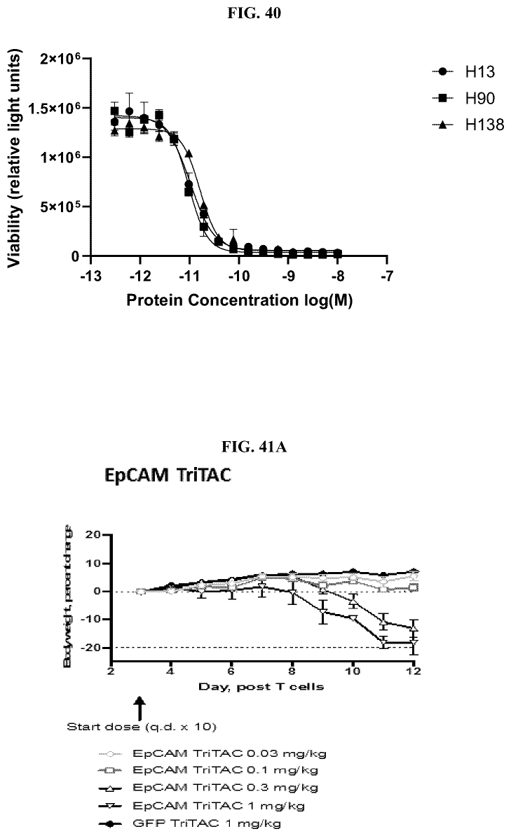

T cell killing potential of the anti-EpCAM/anti-CD3 fusion proteins present in the conditioned medium was assessed as described in Example 16. Results are provided in Table 15 and in FIG. 40.

TABLE 15
EC₅₀ Values for Redirected T Cell Killing of NCI-
H508 Cells by Purified Anti-CD3/Anti-EpCAM Proteins
Containing Humanized Anti-EpCAM Sequences

| Humanized anti-EpCAM | |
|---|---|
| Binder Sequence | EC₅₀ (pM) |
| H13 | 10 |
| H90 | 10 |
| H138 | 16 |

### Example 18: Demonstration of Improved Tolerability in Mouse, Conferred by an Exemplary EpCAM Targeting ProTriTAC Molecule

In this study, the tolerability of an exemplary EpCAM targeting ProTriTAC molecule was assessed. Seven weeks old NSG female tumor free mice were intraperitoneally injected with 2×10⁷ expanded human T cells at the commencement of the study, i.e., at day 0. On day 2, treatment was started by dividing the mice into various groups and administering to them varying concentrations of the exemplary EpCAM targeting ProTriTAC molecule, containing the linker sequence L040, an EpCAMR targeting TriTAC molecule, an EpCAM targeting ProTriTAC molecule containing a non-cleavable linker (EpCAM ProTrⁱTAC (NCLV), and a GFP TriTAC molecule (SEQ ID No. 792) as a control. The molecules were administered once daily for 10 days, at the following dosages: 0.03 mg/kg, 0.1 mg/kg, 0.3 mg/kg, and 1 mg/kg. Starting from day 2, body weight of the animals was recorded daily.

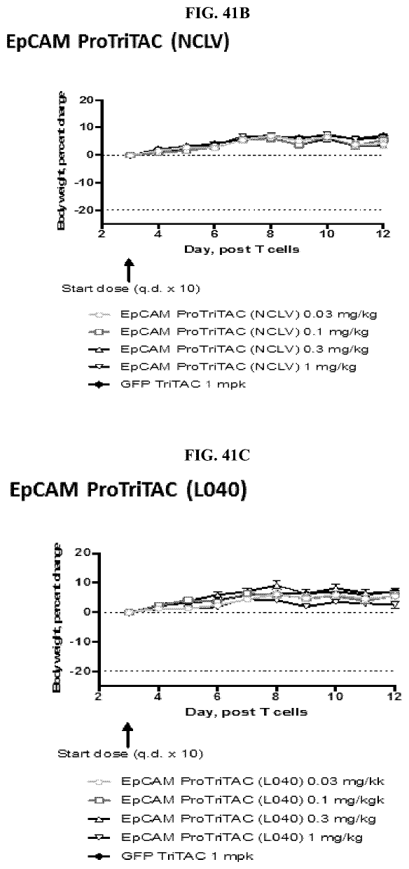

As shown in FIGS. 41A-41C, the EpCAM targeting ProTriTAC molecule containing a non-cleavable linker (ProTriTAC (NCLV)) (SEQ ID No. 908) and a GFP TriTAC (used as a negative control) were very well tolerated in mice even at the highest dose of 1 mg/kg. The EpCAM targeted ProTriTAC molecule containing the linker sequence of L040 (SEQ ID No. 907) was well tolerated at the highest tested dosage of 1 mg/kg, whereas the EpCAM targeted TriTAC (SEQ ID No. 906) was well tolerated 0.1 mg/kg. It was thus observed that the EpCAM targeting ProTriTAC containing the L040 linker sequence conferred at least about 10 times improved tolerability, in mouse, compared to the EpCAM targeting TriTAC.

### Example 19: Xenograft Tumor Model

An EpCAM targeting fusion protein of this disclosure (e.g., a fusion protein which is a trispecific protein comprising an anti-EpCAM heavy chain only single domain antibody, an anti-CD3 scFv, and an anti-Albumin domain) is evaluated in a xenograft model. In order to determine efficacy of the exemplary EpCAM targeting fusion protein in vivo, multiple xenograft tumor models are used. Examples of common tumor cell lines for use in xenograft tumor studies include A549 (non-small cell lung carcinoma) cells, DU-145 (prostate) cells, MCF-7 (breast) cells, Colo 205 (colon) cells, 3T3 (mouse fibroblast) cells, NCI H441 cells, HEP G2 (hepatoma) cells, MDA MB 231 (breast) cells, HT-29 (colon) cells, MDA-MB-435s (breast) cells, U266 cells, SH-SYSY cells, Sk-Mel-2 cells, NCI-H929, RPM18226, and A431 cells. Immune-deficient NOD/scid mice are sub-lethally irradiated (2 Gy) and subcutaneously inoculated with 1×10⁶ tumor cells (e.g., NCI H441 cells) into their right dorsal flank. When tumors reach 100 to 200 mm³, animals are allocated into 3 treatment groups. Groups 2 and 3 are intraperitoneally injected with 1.5×10⁷ activated human T-cells. Three days later, animals from Group 3 are subsequently treated with the exemplary EPCAM targeting-trispecific antigen-binding protein of Groups 1 and 2 are only treated with vehicle. Body weight and tumor volume are determined for 30 days, beginning at least 5 days post treatment with the exemplary EPCAM targeting trispecific protein.

It is expected that animals treated with the exemplary EpCAM targeting trispecific protein have a statistically significant delay in tumor growth in comparison to the respective vehicle-treated control group.

### Example 20: Proof-of-Concept Clinical Trial Protocol for Administration of the EpCAM Targeting Trispecific Antigen-Binding Protein of Example 19 to Ovarian Cancer Patients

This is a Phase I/II clinical trial for studying an exemplary EpCAM targeting trispecific antigen-binding protein of this disclosure as a treatment for an epithelial ovarian cancer.
1. Study Outcomes:
2. Primary: Maximum tolerated dose of the exemplary EpCAM targeting trispecific protein.
3. Secondary: To determine whether in vitro response of the exemplary EpCAM targeting trispecific protein is associated with clinical response

### Phase I

4. The maximum tolerated dose (MTD) will be determined in the phase I section of the trial.
   1.1 The maximum tolerated dose (MTD) will be determined in the phase I section of the trial.
   1.2 Patients who fulfill eligibility criteria will be entered into the trial to EPCAM targeting trispecific proteins of the previous examples.
   1.3 The goal is to identify the highest dose of EpCAM targeting trispecific proteins of the previous examples that can be administered safely without severe or unmanageable side effects in participants. The dose given will depend on the number of participants who have been enrolled in the study prior and how well the dose was tolerated. Not all participants will receive the same dose.

### Phase II

2.1 A subsequent phase II section will be treated at the MTD with a goal of determining if therapy with therapy of the exemplary EpCAM targeting trispecific protein results in at least a 20% response rate.
Primary Outcome for the Phase II—To determine if therapy of EPCAM targeting trispecific protein results in at least 20% of patients achieving a clinical response (blast response, minor response, partial response, or complete response)

### Eligibility:

Histologically or cytologically confirmed epithelial ovarian cancer. May have Recurrent epithelial ovarian carcinoma or disease progression following failure of first-line, platinum-based chemotherapy with no more than one prior platinum based regimen therapy
Adequate laboratory values of bone marrow function, renal function, liver function, and echocardiogram tests

### Phase III

5.3.1A subsequent phase III section will carried out with the exemplary EpCAM targeting trispecific protein, wherein secondary endpoints such as response rate (RR), patient recorded outcomes (PRO), progression-free survival (PFS), duration of progression free survival, time to progression (TIP), overall survival, health-related quality of life assessment, number of participants with overall survival, duration of response, time to response, number of participants with response, and time to tumor growth etc. will be assessed.

### Example 21: In Vitro Assay of ProTriTAC Molecules Cloned with Protease Cleavable Sequences to Determine Percent Cleavage

To determine the degree of activation of protease-cleavable linkers, ProTriTAC molecules were cloned with various protease cleavable sequences in the linker between the anti-ALB domain and the anti-CD3e-scFv domain. The proteins were expressed in Expi293 cells and purified via Protein A chromatography. The linkers were tested for their susceptibility to cleavage by a panel of proteases by mixing the ProTriTAC with 5-100 nM purified protease and incubating at 37° C. for one hour. To quantify the percentage of each ProTriTAC cleaved, it's association rate to huCD3e was measured using Biolayer interferometry (Octet). A standard curve was generated using a mixture of purified anti-CD3e::anti-Target (C3914) and non-cleavable ProTriTAC (C3912). In vitro assays were performed with both EpCAM and EGFR tumor antigen-targeting protease cleavable ProTriTAC molecules. Table 16 summarizes the linker sequences used in the Examples below. Tables 17 and 18 summarize the results obtained with EGFR-targeting protease-cleavable ProTriTAC.

TABLE 16
Linker and protease cleavage sites sequences

| | Protease | Full Linker Sequence | Full Linker Sequence |
|---|---|---|---|
| Linker | Sequence | version 1 | version 2 |
| name | SEQ ID NO: | SEQ ID NO: | SEQ ID NO: |
| L001 | KPLGLQARVV | GGGGKPLGLQARVVGG | GGGGKPLGLQARVVGGGG |
| | (SEQ ID NO: 58) | GGT (SEQ ID NO: 53) | S (SEQ ID NO: 958) |
| L040 | PQASTGRSGG | GGGGPQASTGRSGGGGG | GGGGPQASTGRSGGGGGG |
| | (SEQ ID NO: 59) | GT (SEQ ID NO: 932) | S (SEQ ID NO: 959) |
| L041 | PQGSTGRAAG | GGGGPQGSTGRAAGGG | GGGGPQGSTGRAAGGGGG |
| | (SEQ ID NO: 60) | GGT (SEQ ID NO: 933) | S (SEQ ID NO: 960) |
| L043 | PQGSTARSAG | GGGGPQGSTARSAGGGG | GGGGPQGSTARSAGGGGG |
| | (SEQ ID NO: 909) | GT (SEQ ID NO: 934) | S (SEQ ID NO: 961) |
| L045 | PIPVQGRAH | GGGGPIPVQGRAHGGGG | GGGGPIPVQGRAHGGGGS |
| | (SEQ ID NO: 62) | T (SEQ ID NO: 935) | (SEQ ID NO: 962) |
| L054 | VANLASTGRA | GGGGVANLASTGRAGG | GGGGVANLASTGRAGGGG |
| | (SEQ ID NO: 910) | GGT (SEQ ID NO: 936) | S (SEQ ID NO: 963) |
| L055 | PLGLSTGRA | GGGGPLGLSTGRAGGGG | GGGGPLGLSTGRAGGGGS |
| | (SEQ ID NO: 911) | T (SEQ ID NO: 937) | (SEQ ID NO: 964) |
| L056 | PQASTGRA (SEQ | GGGGPQASTGRAGGGT | GGGGPQASTGRAGGGTS |
| | ID NO: 912) | (SEQ ID NO: 938) | (SEQ ID NO: 965) |
| L057 | PKPQASTGRA | GGGGPKPQASTGRAGGG | GGGGPKPQASTGRAGGGS |
| | (SEQ ID NO: 913) | T (SEQ ID NO: 939) | (SEQ ID NO: 966) |
| L058 | PTGLESTGRA | GGGGPTGLESTGRAGGG | GGGGPTGLESTGRAGGGS |
| | (SEQ ID NO: 914) | T (SEQ ID NO: 940) | (SEQ ID NO: 967) |
| L059 | PENFFSTGRA | GGGGPENFFSTGRAGGG | GGGGPENFFSTGRAGGGS |
| | (SEQ ID NO: 915) | T (SEQ ID NO: 941) | (SEQ ID NO: 968) |
| L060 | AATLKSTGRA | GGGGAATLKSTGRAGG | GGGGAATLKSTGRAGGGS |
| | (SEQ ID NO: 916) | GT (SEQ ID NO: 942) | (SEQ ID NO: 969) |
| L061 | PQDLLSTGRA | GGGGPQDLLSTGRAGGG | GGGGPQDLLSTGRAGGGS |
| | (SEQ ID NO: 917) | T (SEQ ID NO: 943) | (SEQ ID NO: 970) |
| L062 | PAGLSTGRA | GGGGPAGLSTGRAGGGT | GGGGPAGLSTGRAGGGS |
| | (SEQ ID NO: 918) | (SEQ ID NO: 944) | (SEQ ID NO: 971) |
| L063 | VLGLSTGRA | GGGGVLGLSTGRAGGGT | GGGGVLGLSTGRAGGGS |
| | (SEQ ID NO: 919) | (SEQ ID NO: 945) | (SEQ ID NO: 972) |
| L064 | VANLASTGA | GGGGVANLASTGAGGG | GGGGVANLASTGAGGGS |
| | (SEQ ID NO: 920) | T (SEQ ID NO: 946) | (SEQ ID NO: 973) |
| L065 | PLGLSTGA (SEQ | GGGGPLGLSTGAGGGT | GGGGPLGLSTGAGGGS |
| | ID NO: 921) | (SEQ ID NO: 947) | (SEQ ID NO: 974) |
| L066 | PQASTGA (SEQ | GGGGPQASTGAGGGT | GGGGPQASTGAGGGS (SEQ |
| | ID NO: 922) | (SEQ ID NO: 948) | ID NO: 975) |
| L067 | PKPQASTGA | GGGGPKPQASTGAGGGT | GGGGPKPQASTGAGGGS |
| | (SEQ ID NO: 923) | (SEQ ID NO: 949) | (SEQ ID NO: 976) |
| L068 | PTGLESTGA | GGGGPTGLESTGAGGGT | GGGGPTGLESTGAGGGS |
| | (SEQ ID NO: 924) | (SEQ ID NO: 950) | (SEQ ID NO: 977) |
| L069 | PENFFSTGA | GGGGPENFFSTGAGGGT | GGGGPENFFSTGAGGGS |
| | (SEQ ID NO: 925) | (SEQ ID NO: 951) | (SEQ ID NO: 978) |
| L070 | AATLKSTGA | GGGGAATLKSTGAGGGT | GGGGAATLKSTGAGGGS |
| | (SEQ ID NO: 926) | (SEQ ID NO: 952) | (SEQ ID NO: 979) |
| L071 | PQDLLSTGA | GGGGPQDLLSTGAGGGT | GGGGPQDLLSTGAGGGS |
| | (SEQ ID NO: 927) | (SEQ ID NO: 953) | (SEQ ID NO: 980) |
| L072 | PAGLSTGA (SEQ | GGGGPAGLSTGAGGGT | GGGGPAGLSTGAGGGS |
| | ID NO: 928) | (SEQ ID NO: 954) | (SEQ ID NO: 981) |
| L073 | VLGLSTGA (SEQ | GGGGVLGLSTGAGGGT | GGGGVLGLSTGAGGGS |
| | ID NO: 929) | (SEQ ID NO: 955) | (SEQ ID NO: 982) |
| L075 | RTRYEDGGS | GGGGRTRYEDGGSGGGT | GGGGRTRYEDGGSGGGS |
| | (SEQ ID NO: 930) | (SEQ ID NO: 956) | (SEQ ID NO: 983) |
| L076 | PQASTGGSG | GGGGPQASTGGSGGGGT | GGGGPQASTGGSGGGGS |
| | (SEQ ID NO: 931) | (SEQ ID NO: 957) | (SEQ ID NO: 984) |
| protease | VANLA (SEQ ID | | |
| cleavage | NO: 985) | | |
| site | | | |
| protease | PLGL (SEQ ID | | |
| cleavage | NO: 986) | | |
| site | | | |
| protease | PQAS (SEQ ID | | |
| cleavage | NO: 987) | | |
| site | | | |
| protease | PKPQA (SEQ ID | | |
| cleavage | NO: 988) | | |
| site | | | |
| protease | PTGLE (SEQ ID | | |
| cleavage | NO: 989) | | |
| site | | | |
| protease | PENFF (SEQ ID | | |
| cleavage | NO: 990) | | |
| site | | | |
| protease | AATLK (SEQ ID | | |
| cleavage | NO: 991) | | |
| site | | | |
| protease | PQDLL (SEQ ID | | |
| cleavage | NO: 992) | | |
| site | | | |
| protease | PAGL (SEQ ID | | |
| cleavage | NO: 993) | | |
| site | | | |
| protease | VLGL (SEQ ID | | |
| cleavage | NO: 994) | | |
| site | | | |
| protease | RLQLKL (SEQ ID | | |
| cleavage | NO: 11) | | |
| site | | | |
| protease | RTRYED (SEQ ID | | |
| cleavage | NO: 995) | | |
| site | | | |
| protease | KPLGL (SEQ ID | | |
| cleavage | NO: 996) | | |
| site | | | |

TABLE 17
Percent cleavage of EGFR-targeting ProTriTAC following
in vitro activation by purified proteases

| | Linker | | | | | Cathepsin | Neutrophil |
|---|---|---|---|---|---|---|---|
| Protein name | name | PBS | Matripase | uPA | Furin | G | Elastase |
| AxCT mask27 | L001 | 0 | 37 | 0 | 0 | 0 | 34 |
| EGFR EH104 L001 | | | | | | | |
| version 1 (SEQ ID | | | | | | | |
| NO: 1013) | | | | | | | |
| AxCT mask27 | L040 | 0 | 67 | 22 | 0 | 0 | 13 |
| EGFR EH104 L040 | | | | | | | |
| version 1 (SEQ ID | | | | | | | |
| NO: 1014) | | | | | | | |
| AxCT mask27 | L041 | 0 | 62 | 28 | 0 | 0 | 0 |
| EGFR EH104 L041 | | | | | | | |
| version 1 (SEQ ID | | | | | | | |
| NO: 1015) | | | | | | | |
| AxCT mask27 | L043 | 0 | 59 | 40 | 0 | 0 | 7 |
| EGFR EH104 L043 | | | | | | | |
| version 1 (SEQ ID | | | | | | | |
| NO: 1016) | | | | | | | |
| AxCT mask27 | L054 | 0 | 37 | 7 | 0 | 0 | 23 |
| EGFR EH104 L054 | | | | | | | |
| version 1 (SEQ ID | | | | | | | |
| NO: 1018) | | | | | | | |
| AxCT mask27 | L055 | 0 | 53 | 24 | 0 | 0 | 9 |
| EGFR EH104 L055 | | | | | | | |
| version 1 (SEQ ID | | | | | | | |
| NO: 1019) | | | | | | | |
| AxCT mask27 | L056 | 0 | 39 | 8 | 0 | 0 | 14 |
| EGFR EH104 L056 | | | | | | | |
| version 1 (SEQ ID | | | | | | | |
| NO: 1020) | | | | | | | |
| AxCT mask27 | L057 | 0 | 36 | 0 | 0 | 0 | 28 |
| EGFR EH104 L057 | | | | | | | |
| version 1 (SEQ ID | | | | | | | |
| NO: 1021) | | | | | | | |
| AxCT mask27 | L058 | 0 | 23 | 10 | 0 | 0 | 9 |
| EGFR EH104 L058 | | | | | | | |
| version 1 (SEQ ID | | | | | | | |
| NO: 1022) | | | | | | | |
| AxCT mask27 | L059 | 0 | 33 | 9 | 0 | 8 | 15 |
| EGFR EH104 L059 | | | | | | | |
| version 1 (SEQ ID | | | | | | | |
| NO: 1023) | | | | | | | |
| AxCT mask27 | L060 | 0 | 62 | 0 | 0 | 0 | 10 |
| EGFR EH104 L060 | | | | | | | |
| version 1 (SEQ ID | | | | | | | |
| NO: 1024) | | | | | | | |
| AxCT mask27 | L061 | 0 | 20 | 6 | 0 | 0 | 19 |
| EGFR EH104 L061 | | | | | | | |
| version 1 (SEQ ID | | | | | | | |
| NO: 1025) | | | | | | | |
| AxCT mask27 | L062 | 0 | 52 | 13 | 0 | 0 | 8 |
| EGFR EH104 L062 | | | | | | | |
| version 1 (SEQ ID | | | | | | | |
| NO: 1026) | | | | | | | |
| AxCT mask27 | L063 | 0 | 49 | 13 | 0 | 0 | 9 |
| EGFR EH104 L063 | | | | | | | |
| version 1 (SEQ ID | | | | | | | |
| NO: 1027) | | | | | | | |
| AxCT mask27 | L064 | 0 | 0 | 0 | 0 | 0 | 30 |
| EGFR EH104 L064 | | | | | | | |
| version 1 (SEQ ID | | | | | | | |
| NO: 1028) | | | | | | | |
| AxCT mask27 | L065 | 0 | 0 | 0 | 0 | 0 | 10 |
| EGFR EH104 L065 | | | | | | | |
| version 1 (SEQ ID | | | | | | | |
| NO: 1029) | | | | | | | |
| AxCT mask27 | L066 | 0 | 0 | 0 | 0 | 0 | 9 |
| EGFR EH104 L066 | | | | | | | |
| version 1 (SEQ ID | | | | | | | |
| NO: 1030) | | | | | | | |
| AxCT mask27 | L067 | 0 | 0 | 0 | 0 | 0 | 32 |
| EGFR EH104 L067 | | | | | | | |
| version 1 (SEQ ID | | | | | | | |
| NO: 1031) | | | | | | | |
| AxCT mask27 | L068 | 0 | 0 | 0 | 0 | 0 | 19 |
| EGFR EH104 L068 | | | | | | | |
| version 1 (SEQ ID | | | | | | | |
| NO: 1032) | | | | | | | |
| AxCT mask27 | L069 | 0 | 0 | 0 | 0 | 12 | 22 |
| EGFR EH104 L069 | | | | | | | |
| version 1 (SEQ ID | | | | | | | |
| NO: 1033) | | | | | | | |
| AxCT mask27 | L070 | 0 | 13 | 0 | 0 | 0 | 11 |
| EGFR EH104 L070 | | | | | | | |
| version 1 (SEQ ID | | | | | | | |
| NO: 1034) | | | | | | | |
| AxCT mask27 | L071 | 0 | 0 | 0 | 0 | 0 | 26 |
| EGFR EH104 L071 | | | | | | | |
| version 1 (SEQ ID | | | | | | | |
| NO: 1035) | | | | | | | |
| AxCT mask27 | L072 | 0 | 0 | 0 | 0 | 0 | 10 |
| EGFR EH104 L072 | | | | | | | |
| version 1 (SEQ ID | | | | | | | |
| NO: 1036) | | | | | | | |
| AxCT mask27 | L073 | 0 | 0 | 0 | 0 | 0 | 9 |
| EGFR EH104 L073 | | | | | | | |
| version 1 (SEQ ID | | | | | | | |
| NO: 1037) | | | | | | | |

TABLE 18
Percent cleavage of EGFR-targeting ProTriTAC following in
vitro activation by purified matrix metalloproteinases

| | Linker | | | | | | | | |
|---|---|---|---|---|---|---|---|---|---|
| Protein name | name | MMP-1 | MMP-2 | MMP-7 | MMP-8 | MMP-9 | MMP-12 | MMP-13 | MMP-14 |
| AxCT mask27 | L001 | 40 | 71 | 71 | 44 | 74 | 68 | 65 | 54 |
| EGFR EH104 L001 | | | | | | | | | |
| version 1 (SEQ ID | | | | | | | | | |
| NO: 1013) | | | | | | | | | |
| AxCT mask27 | L040 | 6 | 60 | 0 | 6 | 63 | 13 | 30 | 0 |
| EGFR EH104 L040 | | | | | | | | | |
| version 1 (SEQ ID | | | | | | | | | |
| NO: 1014) | | | | | | | | | |
| AxCT mask27 | L041 | 0 | 51 | 0 | 0 | 59 | 0 | 14 | 0 |
| EGFR EH104 L041 | | | | | | | | | |
| version 1 (SEQ ID | | | | | | | | | |
| NO: 1015) | | | | | | | | | |
| AxCT mask27 | L043 | 0 | 27 | 0 | 0 | 20 | 0 | 22 | 0 |
| EGFR EH104 L043 | | | | | | | | | |
| version 1 (SEQ ID | | | | | | | | | |
| NO: 1016) | | | | | | | | | |
| AxCT mask27 | L054 | 0 | 59 | 24 | 8 | 9 | 30 | 29 | 0 |
| EGFR EH104 L054 | | | | | | | | | |
| version 1 (SEQ ID | | | | | | | | | |
| NO: 1018) | | | | | | | | | |
| AxCT mask27 | L055 | 10 | 35 | 67 | 29 | 75 | 72 | 61 | 16 |
| EGFR EH104 L055 | | | | | | | | | |
| version 1 (SEQ ID | | | | | | | | | |
| NO: 1019) | | | | | | | | | |
| AxCT mask27 | L056 | 6 | 51 | 6 | 7 | 52 | 12 | 26 | 0 |
| EGFR EH104 L056 | | | | | | | | | |
| version 1 (SEQ ID | | | | | | | | | |
| NO: 1020) | | | | | | | | | |
| AxCT mask27 | L057 | 7 | 47 | 0 | 11 | 57 | 18 | 31 | 0 |
| EGFR EH104 L057 | | | | | | | | | |
| version 1 (SEQ ID | | | | | | | | | |
| NO: 1021) | | | | | | | | | |
| AxCT mask27 | L058 | 0 | 62 | 0 | 0 | 27 | 9 | 47 | 0 |
| EGFR EH104 L058 | | | | | | | | | |
| version 1 (SEQ ID | | | | | | | | | |
| NO: 1022) | | | | | | | | | |
| AxCT mask27 | L059 | 0 | 39 | 0 | 18 | 32 | 62 | 49 | 0 |
| EGFR EH104 L059 | | | | | | | | | |
| version 1 (SEQ ID | | | | | | | | | |
| NO: 1023) | | | | | | | | | |
| AxCT mask27 | L060 | 0 | 27 | 9 | 0 | 7 | 18 | 13 | 0 |
| EGFR EH104 L060 | | | | | | | | | |
| version 1 (SEQ ID | | | | | | | | | |
| NO: 1024) | | | | | | | | | |
| AxCT mask27 | L061 | 0 | 43 | 26 | 15 | 54 | 49 | 43 | 0 |
| EGFR EH104 L061 | | | | | | | | | |
| version 1 (SEQ ID | | | | | | | | | |
| NO: 1025) | | | | | | | | | |
| AxCT mask27 | L062 | 0 | 63 | 25 | 6 | 63 | 51 | 56 | 18 |
| EGFR EH104 L062 | | | | | | | | | |
| version 1 (SEQ ID | | | | | | | | | |
| NO: 1026) | | | | | | | | | |
| AxCT mask27 | L063 | 0 | 31 | 37 | 0 | 21 | 31 | 23 | 0 |
| EGFR EH104 L063 | | | | | | | | | |
| version 1 (SEQ ID | | | | | | | | | |
| NO: 1027) | | | | | | | | | |
| AxCT mask27 | L064 | 0 | 63 | 28 | 7 | 13 | 34 | 24 | 0 |
| EGFR EH104 L064 | | | | | | | | | |
| version 1 (SEQ ID | | | | | | | | | |
| NO: 1028) | | | | | | | | | |
| AxCT mask27 | L065 | 10 | 71 | 60 | 18 | 70 | 68 | 53 | 0 |
| EGFR EH104 L065 | | | | | | | | | |
| version 1 (SEQ ID | | | | | | | | | |
| NO: 1029) | | | | | | | | | |
| AxCT mask27 | L066 | 6 | 25 | 0 | 0 | 29 | 16 | 21 | 0 |
| EGFR EH104 L066 | | | | | | | | | |
| version 1 (SEQ ID | | | | | | | | | |
| NO: 1030) | | | | | | | | | |
| AxCT mask27 | L067 | 0 | 34 | 0 | 13 | 50 | 26 | 33 | 0 |
| EGFR EH104 L067 | | | | | | | | | |
| version 1 (SEQ ID | | | | | | | | | |
| NO: 1031) | | | | | | | | | |
| AxCT mask27 | L068 | 0 | 48 | 0 | 0 | 19 | 9 | 52 | 0 |
| EGFR EH104 L068 | | | | | | | | | |
| version 1 (SEQ ID | | | | | | | | | |
| NO: 1032) | | | | | | | | | |
| AxCT mask27 | L069 | 0 | 29 | 0 | 16 | 29 | 63 | 51 | 0 |
| EGFR EH104 L069 | | | | | | | | | |
| version 1 (SEQ ID | | | | | | | | | |
| NO: 1033) | | | | | | | | | |
| AxCT mask27 | L070 | 0 | 22 | 9 | 0 | 0 | 19 | 11 | 0 |
| EGFR EH104 L070 | | | | | | | | | |
| version 1 (SEQ ID | | | | | | | | | |
| NO: 1034) | | | | | | | | | |
| AxCT mask27 | L071 | 0 | 39 | 30 | 20 | 46 | 51 | 37 | 0 |
| EGFR EH104 L071 | | | | | | | | | |
| version 1 (SEQ ID | | | | | | | | | |
| NO: 1035) | | | | | | | | | |
| AxCT mask27 | L072 | 0 | 73 | 0 | 0 | 51 | 48 | 56 | 12 |
| EGFR EH104 L072 | | | | | | | | | |
| version 1 (SEQ ID | | | | | | | | | |
| NO: 1036) | | | | | | | | | |
| AxCT mask27 | L073 | 0 | 13 | 27 | 0 | 11 | 31 | 18 | 0 |
| EGFR EH104 L073 | | | | | | | | | |
| version 1 (SEQ ID | | | | | | | | | |
| NO: 1037) | | | | | | | | | |

### Example 22: Selecting Linkers for Further Efficacy Studies

The results of Tables 17 and 18 were then analyzed to determine which linkers should be selected for further efficacy studies using EpCAM-targeting ProTriTAC molecules. A panel tox screen was performed using control linkers and EGFR-targeting ProTriTAC molecules, and the percent of change in body weight in mice was monitored to aid in selection. In particular, tumor free mice were intraperitoneally injected with human T cells at the commencement of the study, i.e., at day 0. On day 2, treatment was started by dividing the mice into various groups and administering to them the exemplary EGFR targeting ProTriTAC molecule containing the linker sequence L040, an exemplary EGFR targeting ProTriTAC molecule containing the linker sequence L041, an exemplary EGFR targeting ProTriTAC molecule containing the linker sequence L001, an exemplary EGFR targeting ProTriTAC molecule containing the linker sequence L043, and an exemplary EGFR targeting TriTAC containing a non-cleavable linker (NCLV) at 1 mg/kg, daily for 10 days. The non-cleavable linker and linker L001 were used as controls for later studies. The results of this tox screen (i.e., the change in the percent body weight of mice) is demonstrated by FIG. 42. This was repeated using a variety of linkers, the results of which are indicated in FIG. 43. FIG. 43A shows the results using linker sequences NCLV (non-cleavable) and L001 as controls, and linker sequences L054, L055, L059, L064, L065, and L069. The latter three sequences differ from the former three only be the deletion of an arginine relative to the former three. FIG. 43B shows the results using linker sequences NCLV and L001 as controls, and L060, L061, L062, L063, L070, L071, L072, L073. Linkers L070, L071, L072 and L073 comprises identical sequences as linkers L060, L061, L062, and L063, respectively, but without an arginine. FIG. 43C demonstrates the results conducted using NCLV, L001, and L040 as controls, and linker sequences L074 and L075.

Following these studies, the results were analyzed to determine which linkers should be selected for further efficacy studies. Linker L040, for example, was selected for further efficacy studies based on the small degree of change in body weight demonstrated in subjects treated with them. Table 19 depicts the EGFR-targeting linkers selected for further efficacy studies using EpCAM-targeting molecules. For two of the selected linkers, a version of that linker with a serine protease removed was also selected for further studies. This included a version of linker L040 with a serine protease removed, indicated in Table 19 as L076, and a version of L063 with the serine protease removed, indicated in Table 19 as L073.

TABLE 19
Proposed linkers to move forward into EpCAM ProTriTAC for Efficacy or Efficacy/Tox Study

| | MMP-1 | MMP-2 | MMP-7 | MMP-8 | MMP-9 | MMP-12 | MMP-13 | MMP-14 | Matripase | uPA |
|---|---|---|---|---|---|---|---|---|---|---|
| L001 | 40 | 71 | 71 | 44 | 74 | 68 | 65 | 54 | 37 | 0 |
| L040 | 6 | 60 | 0 | 6 | 63 | 13 | 30 | 0 | 67 | 22 |
| L076 | | | | | | | | | | |
| L059 | 0 | 39 | 0 | 18 | 32 | 62 | 49 | 0 | 33 | 9 |
| L060 | 0 | 27 | 9 | 0 | 7 | 18 | 13 | 0 | 62 | 0 |
| L061 | 0 | 43 | 26 | 15 | 54 | 49 | 43 | 0 | 20 | 6 |
| L063 | 0 | 31 | 37 | 0 | 21 | 31 | 23 | 0 | 49 | 13 |
| L064 | 0 | 63 | 28 | 7 | 13 | 34 | 24 | 0 | 0 | 0 |
| L073 | 0 | 13 | 27 | 0 | 11 | 31 | 18 | 0 | 0 | 0 |
| L075 | | | | | | | | | | |

### Example 23: In Vitro Assay of EpCAM ProTriTAC Molecules Cloned with Protease Cleavable Sequences to Determine Percent Cleavage

Table 20 and 21 summarizes the results for the in vitro assay, conducted using the method of Example 21, with EpCAM-targeting ProTriTAC molecules.

TABLE 20
Percent cleavage of EpCAM-targeting ProTriTAC following
in vitro activation by purified proteases

| Protein Name | PBS control | Matriptase | uPA | Cathepsin | Elastase |
|---|---|---|---|---|---|
| AxCT mask27 EpCAM | 0 | 93 | 39 | 0 | 9 |
| H90.2 L040 version 1 | | | | | |
| (SEQ ID NO: 999) | | | | | |
| AxCT mask27 EpCAM | 0 | 80 | 43 | 0 | 0 |
| H90.2 L041 version 1 | | | | | |
| (SEQ ID NO: 1000) | | | | | |
| AxCT mask27 EpCAM | 0 | 79 | 61 | 0 | 0 |
| H90.2 L043 version 1 | | | | | |
| (SEQ ID NO: 1001) | | | | | |
| AxCT mask27 EpCAM | 0 | 0 | 0 | 0 | 6 |
| H90.2 L076 version 1 | | | | | |
| (SEQ ID NO: 1002) | | | | | |
| AxCT mask27 EpCAM | 0 | 39 | 16 | 6 | 14 |
| H90.2 L059 version 1 | | | | | |
| (SEQ ID NO: 1003) | | | | | |
| AxCT mask27 EpCAM | 0 | 92 | 9 | 0 | 7 |
| H90.2 L060 version 1 | | | | | |
| (SEQ ID NO: 1004) | | | | | |
| AxCT mask27 EpCAM | 0 | 22 | 11 | 0 | 14 |
| H90.2 L061 version 1 | | | | | |
| (SEQ ID NO: 1005) | | | | | |
| AxCT mask27 EpCAM | 0 | 68 | 19 | 0 | 0 |
| H90.2 L063 version 1 | | | | | |
| (SEQ ID NO: 1006) | | | | | |
| AxCT mask27 EpCAM | 0 | 0 | 0 | 0 | 32 |
| H90.2 L064 version 1 | | | | | |
| (SEQ ID NO: 1007) | | | | | |
| AxCT mask27 EpCAM | 0 | 0 | 0 | 0 | 10 |
| H90.2 L073 version 1 | | | | | |
| (SEQ ID NO: 1008) | | | | | |
| AxCT mask27 EpCAM | 0 | 0 | 0 | 8 | 0 |
| H90.2 L075 version 1 | | | | | |
| (SEQ ID NO: 1009) | | | | | |
| AxCT mask27 EpCAM | 0 | 42 | 0 | 0 | 44 |
| H90 L001 version 1 | | | | | |
| (SEQ ID NO: 1010) | | | | | |
| AxCT mask27 EpCAM | 0 | 0 | 0 | 0 | 0 |
| H90.2 NCLV (SEQ ID | | | | | |
| NO: 1011) | | | | | |

TABLE 21
Percent cleavage of EpCAM-targeting ProTriTAC following
in vitro activation by matrix metalloproteinases

| | MMP- | MMP- | MMP- | MMP- | MMP- | MMP- | MMP- | MMP- |
|---|---|---|---|---|---|---|---|---|
| Protein Name | 1 | 2 | 7 | 8 | 9 | 12 | 13 | 14 |
| AxCT mask27 EpCAM | 0 | 90 | 0 | 0 | 60 | 0 | 43 | 0 |
| H90.2 L040 version 1 | | | | | | | | |
| (SEQ ID NO: 999) | | | | | | | | |
| AxCT mask27 EpCAM | 0 | 77 | 0 | 0 | 57 | 0 | 20 | 0 |
| H90.2 L041 version 1 | | | | | | | | |
| (SEQ ID NO: 1000) | | | | | | | | |
| AxCT mask27 EpCAM | 0 | 39 | 0 | 0 | 7 | 0 | 34 | 0 |
| H90.2 L043 version 1 | | | | | | | | |
| (SEQ ID NO: 1001) | | | | | | | | |
| AxCT mask27 EpCAM | 0 | 35 | 0 | 0 | 17 | 0 | 35 | 0 |
| H90.2 L076 version 1 | | | | | | | | |
| (SEQ ID NO: 1002) | | | | | | | | |
| AxCT mask27 EpCAM | 0 | 55 | 0 | 25 | 19 | 71 | 82 | 0 |
| H90.2 L059 version 1 | | | | | | | | |
| (SEQ ID NO: 1003) | | | | | | | | |
| AxCT mask27 EpCAM | 0 | 40 | 9 | 0 | 0 | 16 | 18 | 0 |
| H90.2 L060 version 1 | | | | | | | | |
| (SEQ ID NO: 1004) | | | | | | | | |
| AxCT mask27 EpCAM | 0 | 74 | 35 | 25 | 36 | 64 | 77 | 0 |
| H90.2 L061 version 1 | | | | | | | | |
| (SEQ ID NO: 1005) | | | | | | | | |
| AxCT mask27 EpCAM | 0 | 36 | 48 | 0 | 8 | 33 | 34 | 0 |
| H90.2 L063 version 1 | | | | | | | | |
| (SEQ ID NO: 1006) | | | | | | | | |
| AxCT mask27 EpCAM | 0 | 79 | 32 | 8 | 0 | 36 | 45 | 0 |
| H90.2 L064 version 1 | | | | | | | | |
| (SEQ ID NO: 1007) | | | | | | | | |
| AxCT mask27 EpCAM | 0 | 17 | 43 | 0 | 0 | 36 | 31 | 0 |
| H90.2 L073 version 1 | | | | | | | | |
| (SEQ ID NO: 1008) | | | | | | | | |
| AxCT mask27 EpCAM | 0 | 0 | 0 | 0 | 0 | 0 | 0 | 0 |
| H90.2 L075 version 1 | | | | | | | | |
| (SEQ ID NO: 1009) | | | | | | | | |
| AxCT mask27 EpCAM | 48 | 104 | 92 | 61 | 80 | 91 | 95 | 93 |
| H90 L001 version 1 | | | | | | | | |
| (SEQ ID NO: 1010) | | | | | | | | |
| AxCT mask27 EpCAM | 0 | 0 | 0 | 0 | 0 | 0 | 0 | 0 |
| H90.2 NCLV (SEQ ID | | | | | | | | |
| NO: 1011) | | | | | | | | |

### Example 24: ProTriTACs with L276 have Less Pre-Cleaved Active Drug and are More Manufacturable than proTriTACs with L040 when Produced in CHO Cells

In this example, linker variants were evaluated in reducing the presence of pre-cleaved active drug and improve manufacturability were assessed. 2e⁶ CHOSource™ CHO-K1 GS null cells (Horizon) were nucleofected with 5 μg linearized expression vector plasmid DNA harboring ProTriTAC sequences comprising L040 or L276 protease cleavable linkers. Cells were passaged in medium containing glutamine for 2 days then switched to selection medium lacking glutamine and supplemented with 50 μM methionine sulfoximine (MSX) for up to three weeks such that stable pools were allowed to recover until they reached >95% viability with less than 24 hr doubling time and then banked.

Stable pools were thawed, expanded in glutamine free media supplemented with 50 μM MSX for two to three passages, and seeded into production media at 0.5e⁶ viable cells/ml in Optimum Growth™ Flasks (Thomson) shaken at 150 rpm, 5% CO₂, 60% relative humidity, and 37° C. Fed-batch cultures were supplemented with feeds and glucose on days 3, 5, and 7 and viability and cell density determined by ViCell. Conditioned media (CM) was harvested on day 10 by centrifugation and sterile filtration. Titer was determined by biolayer interferometry using an OctetRed96 and Protein A tips with binding rates compared against a standard curve prepared from a ProTriTAC of known concentration as reference. CM samples were heated in non-reducing sample buffer and separated on NuPage TRIS-Glycine gels, stained with SimplyBlue Safestain (Invitrogen), and imaged with an Azure c500 near infrared imaging system.

Table 22 and FIG. 44 demonstrates a ProTriTAC has substantially reduced pre-cleaved active drug in CHO productions with L276 versus L040 cleavable linker.

TABLE 22
Comparison of 10 d harvest % viability and ProA titer of ProTriTAC
sequences comprising L040 or L276 protease cleavable linkers

| SDS-PAGE | ProTriTAC/ | 10 d Harvest | 10 d ProA |
|---|---|---|---|
| Lane | linker | % Viability | Titer (mg/L) |
| 1 | H90.2/L040 | 80 | 394 |
| 2 | H90.2/L276 | 89 | 419 |

### Example 25: Constructs and Testing for MMP Selectivity

### Constructs

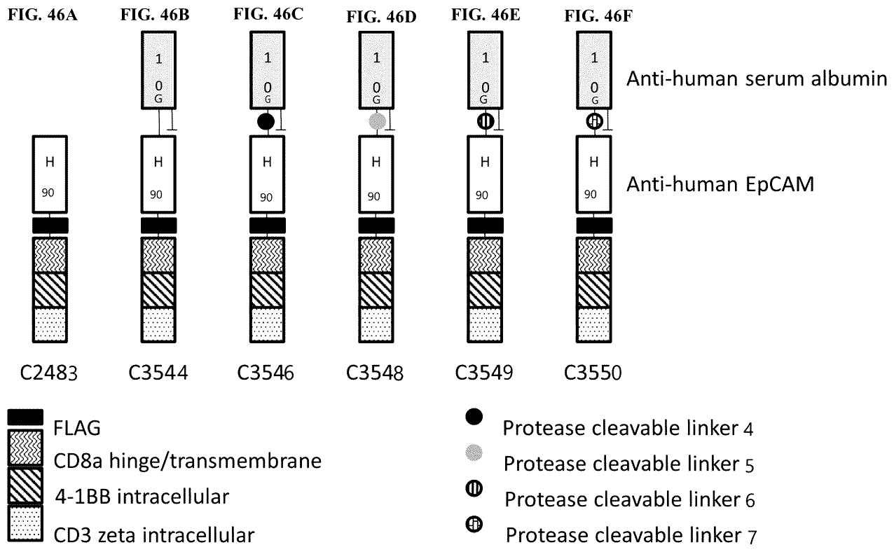

The following ProCAR constructs were made. Construct C2483 includes an anti-human EpCAM sdAb, a FLAG epitope, a CD8 hinge/transmembrane domain, a 4-1BB intracellular domain, and a CD3 zeta intracellular domain (SEQ ID NO: 1075; FIG. 46A). Construct C3544 includes an anti-human serum albumin sdAb, an anti-human EpCAM sdAb, a FLAG epitope, a CD8 hinge/transmembrane domain, a 4-1BB intracellular domain, and a CD3 zeta intracellular domain (SEQ ID NO: 1076; FIG. 46B). Construct C3546 includes an anti-human serum albumin sdAb that contains a mask in the CC′ loop, an anti-human EpCAM sdAb, a protease cleavable linker 4 (SEQ ID NO: 1077), a FLAG epitope, a CD8 hinge/transmembrane domain, a 4-1BB intracellular domain, and a CD3 zeta intracellular domain (SEQ ID NO: 1078; FIG. 46C). Construct C3548 includes an anti-human serum albumin sdAb that contains a mask in the CC′ loop, an anti-human EpCAM sdAb, a protease cleavable linker 5 (SEQ ID NO: 1079), a FLAG epitope, a CD8 hinge/transmembrane domain, a 4-1BB intracellular domain, and a CD3 zeta intracellular domain (SEQ ID NO: 1080; FIG. 46D). Construct C3549 includes an anti-human serum albumin sdAb that contains a mask in the CC′ loop, an anti-human EpCAM sdAb, a protease cleavable linker 6 (SEQ ID NO: 1081), a FLAG epitope, a CD8 hinge/transmembrane domain, a 4-1BB intracellular domain, and a CD3 zeta intracellular domain (SEQ ID NO: 1082; FIG. 46E). Construct C3550 which includes an anti-human serum albumin sdAb that contains a mask in the CC′ loop, an anti-human EpCAM sdAb, a protease cleavable linker 7 (SEQ ID NO: 1083), a FLAG epitope, a CD8 hinge/transmembrane domain, a 4-1BB intracellular domain, and a CD3 zeta intracellular domain (SEQ ID NO: 1084; FIG. 46F).

Pan-MAJP Substrates are not Stable when Expressed on T Cells in ProCAR Format.

T cells were infected with lentivirus made from the indicated constructs to generate CAR-T cells, which were subsequently stained with anti-FLAG antibodies and EpCAM-Fc along with fluorescently labeled secondary antibodies and analyzed by flow cytometry.

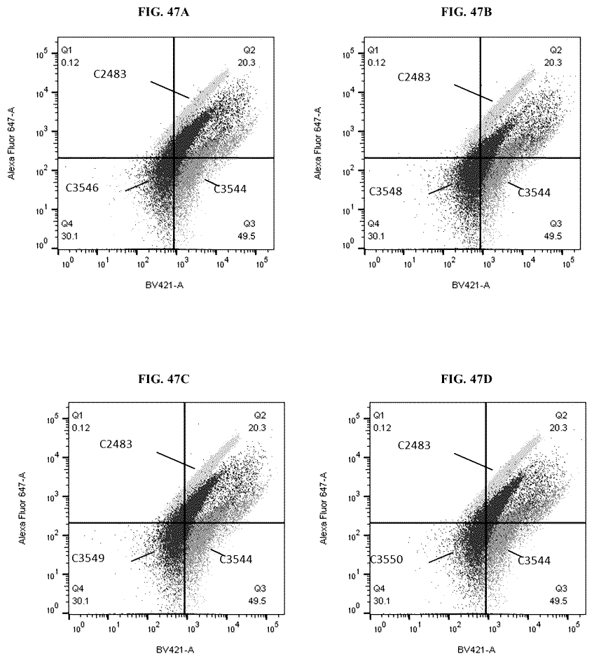

FIG. 47 shows dot plots of M2 staining (x-axis; BV421 channel) and EpCAM-Fc staining (y-axis; AlexaFluor 647 channel) of CAR-T cells. FIG. 47A provides results for constructs C2483, C3546, and C3544. FIG. 47B provides results for constructs C2483, C3548, and C3544. FIG. 47C provides results for constructs C2483, C3549, and C3544. FIG. 47D provides results for constructs C2483, C3550, and C3544.

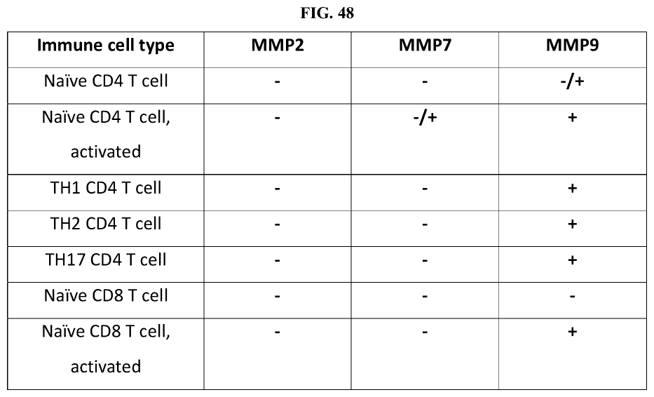

FIG. 48 illustrates that MMP2 and MMP7 are expressed at very low levels in T cells compared to MMP9.

### Example 26: Constructs and Testing for MMP Selectivity

### Constructs

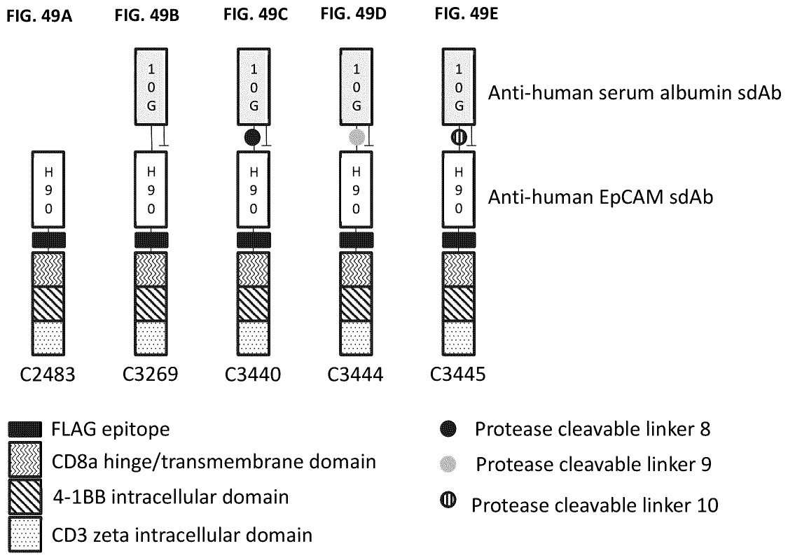

The following ProCAR constructs were made: Construct C2483 which includes an anti-human EpCAM sdAb, a FLAG epitope, a CD8 hinge/transmembrane domain, a 4-1BB intracellular domain, and a CD3 zeta intracellular domain (SEQ ID NO: 1075; FIG. 49A). Construct C3269 includes an anti-human serum albumin sdAb that contains a mask in the CC′ loop, an anti-human EpCAM sdAb, a FLAG epitope, a CD8 hinge/transmembrane domain, a 4-1BB intracellular domain, and a CD3 zeta intracellular domain (SEQ ID NO: 1085; FIG. 49B). Construct C3440 which includes an anti-human serum albumin sdAb that contains a mask in the CC′ loop, an anti-human EpCAM sdAb, a protease cleavable linker 8 (SEQ ID NO: 1086), a FLAG epitope, a CD8 hinge/transmembrane domain, a 4-1BB intracellular domain, and a CD3 zeta intracellular domain (SEQ ID NO: 1087; FIG. 49C). Construct C3444 which includes an anti-human serum albumin sdAb that contains a mask in the CC′ loop, an anti-human EpCAM sdAb, a protease cleavable linker 9 (SEQ ID NO: 1088), a FLAG epitope, a CD8 hinge/transmembrane domain, a 4-1BB intracellular domain, and a CD3 zeta intracellular domain (SEQ ID NO: 1088; FIG. 49D). Construct C3445 which includes an anti-human serum albumin sdAb that contains a mask in the CC′ loop, an anti-human EpCAM sdAb, a protease cleavable linker 10 (SEQ ID NO: 1089), a FLAG epitope, a CD8 hinge/transmembrane domain, a 4-1BB intracellular domain, and a CD3 zeta intracellular domain (SEQ ID NO: 1103; FIG. 49E).

MMP2-Selective Substrates Show Increased Stability when Expressed on T Cells in ProCAR Format

T cells were infected with lentivirus made from the indicated constructs to generate CAR-T cells, which were subsequently stained with anti-FLAG antibodies and EpCAM-Fc along with fluorescently labeled secondary antibodies and analyzed by flow cytometry.

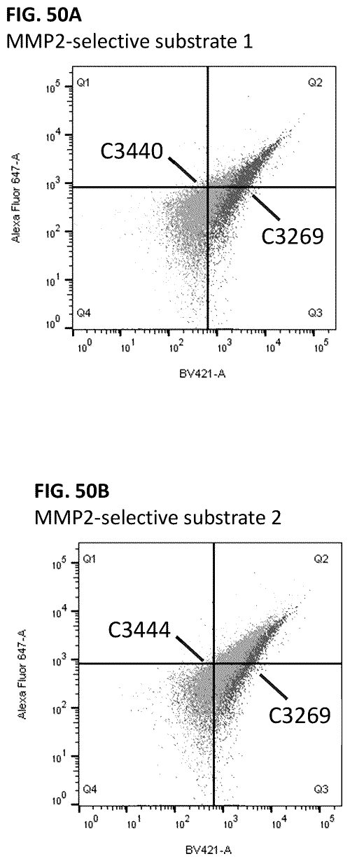

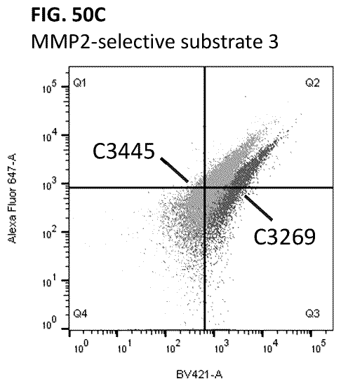

FIG. 50 shows dot plots of M2 staining (x-axis; BV421 channel) and EpCAM-Fc staining (y-axis; AlexaFluor 647 channel) of CAR-T cells. FIG. 50A provides results for constructs C3440 and C3269 for MMP2-selective substrate 1. FIG. 50B provides results for constructs C3444 and C3269 for MMP2-selective substrate 2. FIG. 50C provides results for constructs C3445 and C3269 for MMP2-selective substrate 3.

MMP2-selective substrates show increased stability when expressed on T cells in ProCAR format.

### Example 27: Constructs and Testing for MMP Selectivity

### Constructs

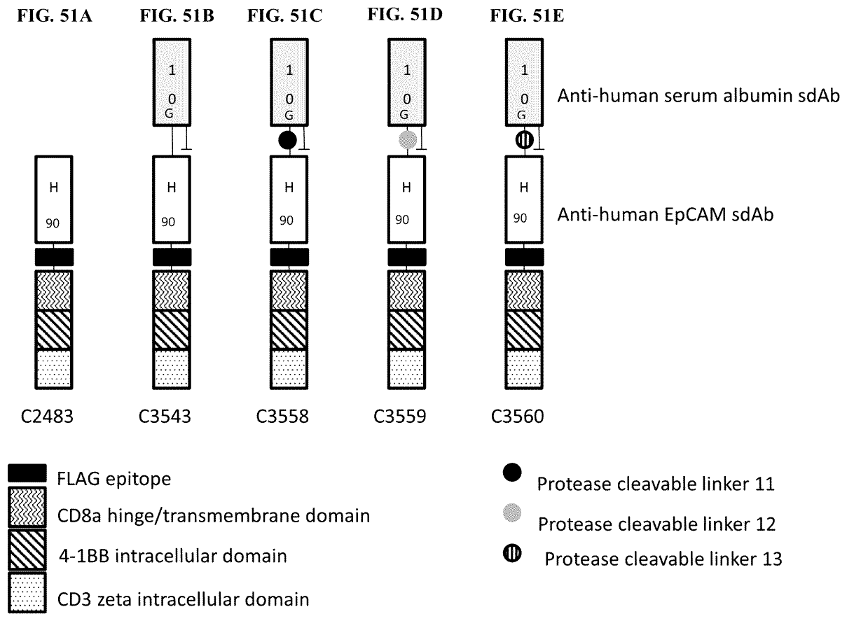

The following ProCAR constructs were made: Construct C2483 which includes an anti-human EpCAM sdAb, a FLAG epitope, a CD8 hinge/transmembrane domain, a 4-1BB intracellular domain, and a CD3 zeta intracellular domain (SEQ ID NO: 1075; FIG. 51A). Construct C3543 includes an anti-human serum albumin sdAb that contains a mask in the CC′ loop, an anti-human EpCAM sdAb, a FLAG epitope, a CD8 hinge/transmembrane domain, a 4-1BB intracellular domain, and a CD3 zeta intracellular domain (SEQ ID NO: 1090; FIG. 51B). Construct C3558 which includes an anti-human serum albumin sdAb that contains a mask in the CC′ loop, an anti-human EpCAM sdAb, a protease cleavable linker 11 (SEQ ID NO: 1091), a FLAG epitope, a CD8 hinge/transmembrane domain, a 4-1BB intracellular domain, and a CD3 zeta intracellular domain (SEQ ID NO: 1092; FIG. 51C). Construct C3559 which includes an anti-human serum albumin sdAb that contains a mask in the CC′ loop, an anti-human EpCAM sdAb, a protease cleavable linker 12 (SEQ ID NO: 1093), a FLAG epitope, a CD8 hinge/transmembrane domain, a 4-1BB intracellular domain, and a CD3 zeta intracellular domain (SEQ ID NO: 1094; FIG. 51D). Construct C3560 which includes an anti-human serum albumin sdAb that contains a mask in the CC′ loop, an anti-human EpCAM sdAb, a protease cleavable linker 13 (SEQ ID NO: 1095), a FLAG epitope, a CD8 hinge/transmembrane domain, a 4-1BB intracellular domain, and a CD3 zeta intracellular domain (SEQ ID NO: 1096; FIG. 51E).

MMP-7-Selective Substrates Show Increased Stability when Expressed on T Cells in ProCAR Format

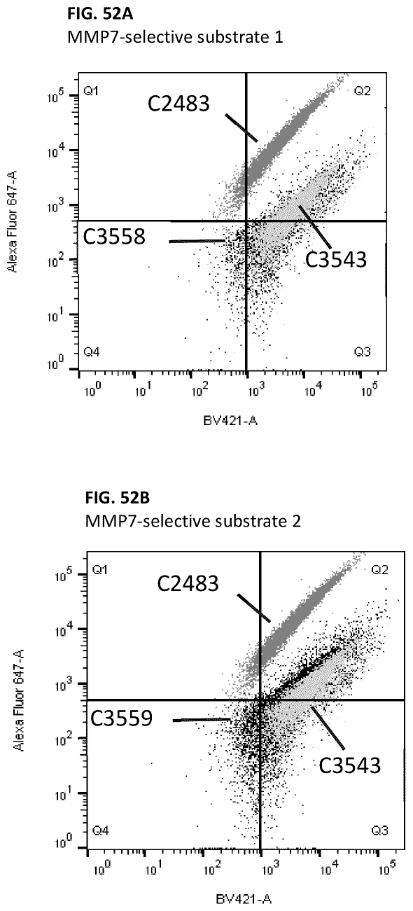

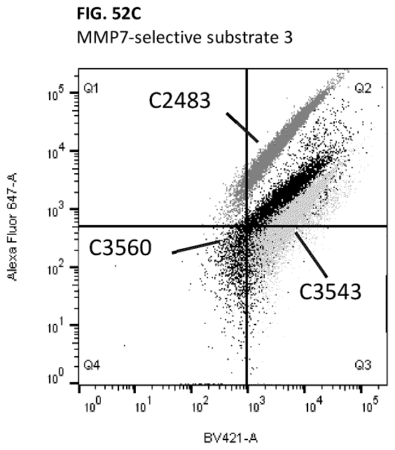

T cells were infected with lentivirus made from the indicated constructs to generate CAR-T cells, which were subsequently stained with anti-FLAG antibodies and EpCAM-Fc along with fluorescently labeled secondary antibodies and analyzed by flow cytometry. Shown above are dot plots of M2 staining (x-axis; BV421 channel) and EpCAM-Fc staining (y-axis; AlexaFluor 647 channel) of CAR-T cells. FIG. 52A provides results for constructs C2483, C3558, and C3543 for MMP7-selective substrate 1. FIG. 52B provides results for constructs C2483, C3559, and C3543 for MMP7-selective substrate 2. FIG. 52C provides results for constructs C2483, C3560, and C3543 for MMP7-selective substrate 3.

MMP-7-selective substrates show increased stability when expressed on T cells in ProCAR format.

MMP7-Selective Substrates are Cleavable with MMP7

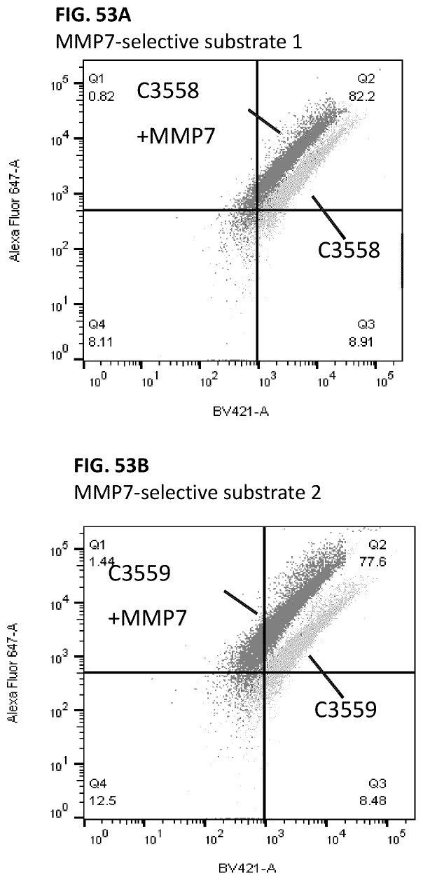

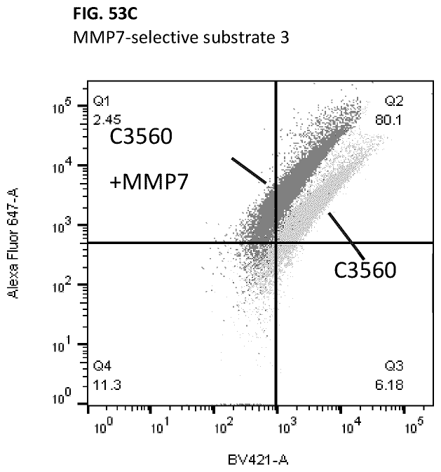

T cells were infected with lentivirus made from the indicated constructs to generate CAR-T cells and either treated with recombinant MMP7 or buffer alone for 1 hour (hr) at 37° C. The CAR-T cells were subsequently stained with anti-FLAG antibodies and EpCAM-Fc along with fluorescently labeled secondary antibodies and analyzed by flow cytometry. Shown above are dot plots of M2 staining (x-axis; BV421 channel) and EpCAM-Fc staining (y-axis; AlexaFluor 647 channel) of CAR-T cells. FIG. 53A provides results for constructs C3558 and C3558+MMP7 for MMP7-selective substrate 1. FIG. 53B provides results for constructs C3559 and C3559+MMP7 for MMP7-selective substrate 2. FIG. 53C provides results for constructs C3560 and C3560+MMP7 for MMP7-selective substrate 3.

### Example 28: Construction and Testing of Exemplary Multivalent Target Binding Proteins

### Constructs

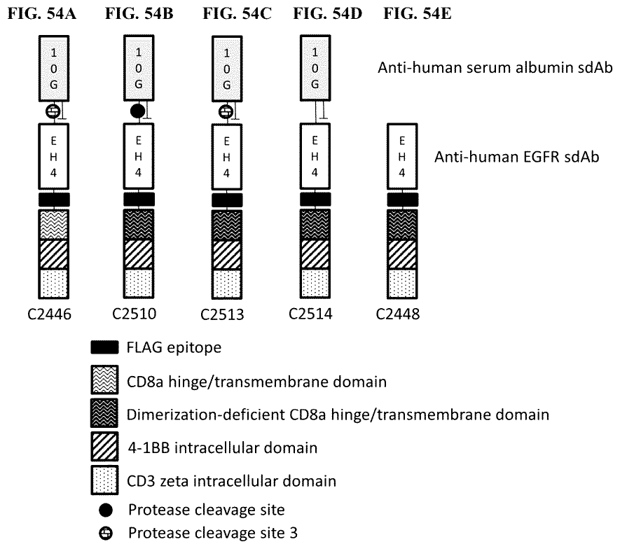

The following constructs were made. Construct C2446 includes an anti-human serine albumin sdAb, a protease cleavage site 3, an anti-human EGFR sdAb, a FLAG epitope, a CD8 hinge/transmembrane domain, a 4-1BB intracellular domain, and a CD3 zeta intracellular domain (FIG. 54A; SEQ ID NO: 1097). Construct C2510 includes an anti-human serine albumin sdAb, a protease cleavage site, an anti-human EGFR sdAb, a FLAG epitope, a dimerization-deficient CD8a hinge/transmembrane domain, a 4-1BB intracellular domain, and a CD3 zeta intracellular domain (FIG. 54B; SEQ ID NO: 1098). Construct C2513 includes an anti-human serine albumin sdAb, a protease cleavage site 3, an anti-human EGFR sdAb, a FLAG epitope, a dimerization-deficient CD8a hinge/transmembrane domain, a 4-1BB intracellular domain, and a CD3 zeta intracellular domain (FIG. 54C; SEQ ID NO: 1099).Construct C2514 includes an anti-human serine albumin sdAb, an anti-human EGFR sdAb, a FLAG epitope, a dimerization-deficient CD8a hinge/transmembrane domain, a 4-1BB intracellular domain, and a CD3 zeta intracellular domain (FIG. 54D; SEQ ID NO: 1100). Construct C2448, includes an anti-human EGFR sdAb, a FLAG epitope, a dimerization-deficient CD8a hinge/transmembrane domain, a 4-1BB intracellular domain, and a CD3 zeta intracellular domain (FIG. 54E; SEQ ID NO: 1101).

### In Vitro Protease Site-Dependent Cell Killing Activity

In a first assay, 300,000 primary human T cells isolated from healthy donors were infected with 1 mL lentiviral supernatant made from the indicated constructs to generate anti-EGFR CAR-T cells. Twenty-five thousand CAR-T cells were subsequently co-cultured under standard conditions at a 10:1 ratio (CAR-T:Target cells) with EGF-expressing cancer cells that stably express luciferase for 72 hours. Luciferase activity is determined as a proxy for cancer cell viability and normalized to the control CAR-T cells that do not contain scFv.

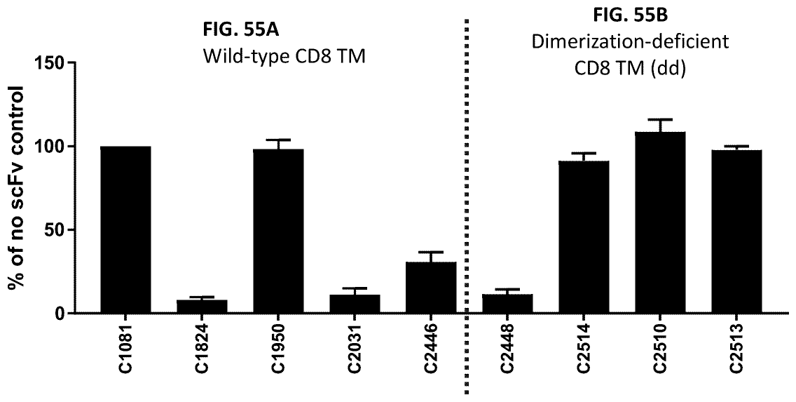

The data demonstrated that the level of protease side-dependent cell killing activity was reduced in ProCAR constructs that lacked the dimerization activity of the CD8a transmembrane domain (FIG. 55B) compared to wild-type (FIG. 55A). Cell killing activity occurs without adding protease. It is believed that the exogenous protease activity comes from the CAR-T cells themselves. The dimerization mutation reduces the amount of cell killing activity in the absence of exogenous protease (e.g., compare C2031 to C2510, or compare C2446 to C2513). Reducing the amount of activation of the ProCAR from T cell proteases may expand the therapeutic window of a given ProCAR.

In a second assay, 300,000 primary human T cells isolated from healthy donors were infected with 1 mL lentiviral supernatant made from the indicated constructs to generate anti-EGFR CAR-T cells, which were subsequently co-cultured at ratios of 10:1, 5:1, 2.5:1, and 1:1 (CAR-T:Target cells) with EGF-expressing cancer cells that stably express luciferase for 72 hours. Luciferase activity is determined as a proxy for cancer cell viability and normalized to the control CAR-T cells that do not contain scFv.

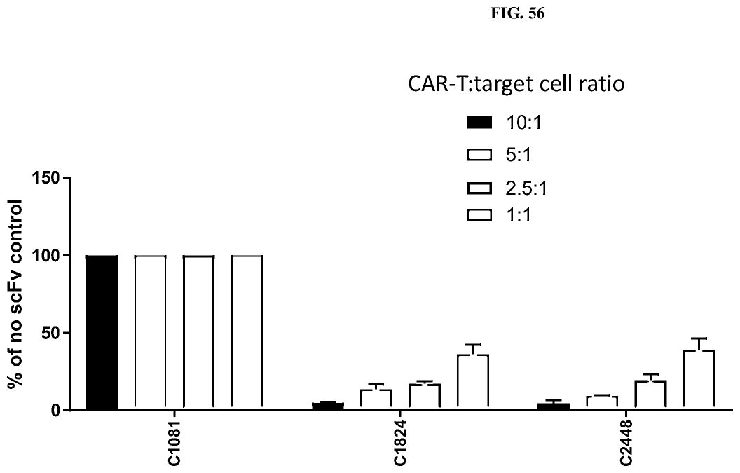

The data demonstrates that dimerization-deficient EGFR CAR has similar killing activity compared to wild-type (FIG. 56).

### In Vivo Protease Site-Dependent Cell Killing Activity

Five NSG mice per group (The Jackson Laboratories, Bar Harbor, ME) were subcutaneously implanted with admixture of 5×10⁶ HCT116 cancer cells and the indicated CAR-T cells (C1081, C1950, or C1824) at a ratio of 1:1 and tumor volumes were measured at the indicated days post-implantation. Significance was determined by using unpaired t test between groups. *** denotes p<0.0005.

These data provide an in vivo demonstration of masking of the anti-EGFR binding domain by the mask in the CC′ loop of the anti-ALB domain using non-cleavable ProCAR. Significance was determined by using unpaired t test between groups. *** denotes p<0.0005.

### Example 29: Testing Efficacy of Different Cleavable Linkers on Reducing Tumor Growth

Epcam targeting ProTriTAC molecules (Epcam H90 Pro with protease cleavable linker L040, EpCAM H90 ProTriTAC with protease cleavable linker 11, EpCAM H90 ProTriTAC with protease cleavable linker 12, and EpCAM H90 ProTriTAC with protease cleavable linker 77) were made comprising different protease cleavable linkers (L040,SEQ ID NO: 787; protease cleavable linker 11, SEQ ID NO: 1091; protease cleavable linker 12, SEQ ID NO: 1093; and protease cleavable linker 77, SEQ ID NO: 1095). All animal experiments were conducted according to the protocol approved by Institutional Animal Care and Use Committee of Harpoon Therapeutics (protocol number HAR-001-2019). Animals were purchased from The Jackson Laboratory then housed in a pathogen free animal facility located at Harpoon Therapeutics in accordance with IACUC guidelines. All studies were performed in NSG™ (NOD-scid IL2Rgammanull) female mice 8 weeks of age with n=5-10 mice per group.

An admixture of HT-29 human colorectal adenocarcinoma tumor cells (5E6) and activated and expanded human T cells (2.5E⁶) was implanted subcutaneously on the right flank of NSG™ mice, followed 5 days later by treatment. Mice with established tumors (average of 215 mm³) were administered a repeat intraperitoneal dose (q.d.×14) of non-targeting GFP TriTAC (at 0.1 mg/kg) or EpCAM targeting ProTriTACs (at 0.1 mg/kg or at 0.3 mg/kg). Tumor growth was monitored at least twice weekly as indicated. All data shown as mean±SEM. Statistics represent RM one-way ANOVA with Dunnett post-hoc test, all groups compared to control non-targeting GFP TriTAC (****, P<0.0001).

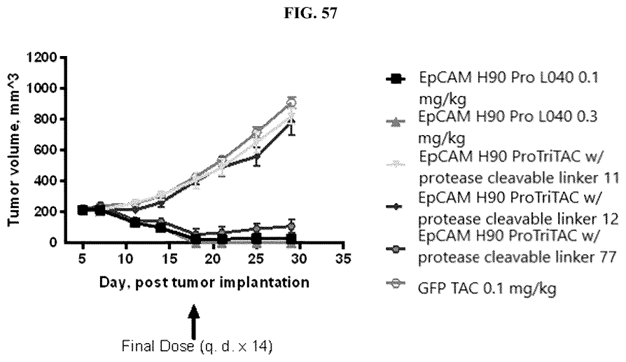

FIG. 57 shows all Epcam targeting ProTriTAC molecules comprising a cleavable linker were able to reduce tumor growth at both doses.

SEQUENCE TABLE

| DESCRIPTION | SEQUENCE |
|---|---|
| Protease: MMP7 | KRALGLPG |
| (SEQ ID NO: 1) | |
| Protease: MMP7 | (DE)8RPLALWRS(DR)8 |
| (SEQ ID NO: 2) | |
| Protease: MMP9 | PR(S/T)(L/I)(S/T) |
| (SEQ ID NO: 3) | |
| Protease: MMP9 | LEATA |
| (SEQ ID NO: 4) | |
| Protease: MMP11 | GGAANLVRGG |
| (SEQ ID NO: 5) | |
| Protease: MMP14 | SGRIGFLRTA |
| (SEQ ID NO: 6) | |
| Protease: MMP | PLGLAG |
| (SEQ ID NO: 7) | |
| Protease: MMP | PLGLAX |
| (SEQ ID NO: 8) | |
| Protease: MMP | PLGC(me)AG |
| (SEQ ID NO: 9) | |
| Protease: MMP | ESPAYYTA |
| (SEQ ID NO: 10) | |
| Protease: MMP | RLQLKL |
| (SEQ ID NO: 11) | |
| Protease: MMP | RLQLKAC |
| (SEQ ID NO: 12) | |
| Protease: MMP2, MMP9, | EP(Cit)G(Hof)YL |
| MMP14 | |
| (SEQ ID NO: 13) | |
| Protease: Urokinase | SGRSA |
| plasminogen activator | |
| (uPA) | |
| (SEQ ID NO: 14) | |
| Protease: Urokinase | DAFK |
| plasminogen activator | |
| (uPA) | |
| (SEQ ID NO: 15) | |
| Protease: Urokinase | GGGRR |
| plasminogen activator | |
| (uPA) | |
| (SEQ ID NO: 16) | |
| Protease: Lysosomal | GFLG |
| Enzyme | |
| (SEQ ID NO: 17) | |
| Protease: Lysosomal | ALAL |
| Enzyme | |
| (SEQ ID NO: 18) | |
| Protease: Lysosomal | FK |
| Enzyme | |
| (SEQ ID NO: 19) | |
| Protease: Cathepsin B | NLL |
| (SEQ ID NO: 20) | |
| Protease: Cathepsin D | PIC(Et)FF |
| (SEQ ID NO: 21) | |
| Protease: Cathepsin K | GGPRGLPG |
| (SEQ ID NO: 22) | |
| Protease: Prostate | HSSKLQ |
| Specific Antigen | |
| (SEQ ID NO: 23) | |
| Protease: Prostate | HSSKLQL |
| Specific Antigen | |
| (SEQ ID NO: 24) | |
| Protease: Prostate | HSSKLQEDA |
| Specific Antigen | |
| (SEQ ID NO: 25) | |
| Protease: Herpes | LVLASSSFGY |
| Simplex Virus Protease | |
| (SEQ ID NO: 26) | |
| Protease: HIV Protease | GVSQNYPIVG |
| (SEQ ID NO: 27) | |
| Protease: CMV Protease | GVVQASCRLA |
| (SEQ ID NO: 28) | |
| Protease: Thrombin | F(Pip)RS |
| (SEQ ID NO: 29) | |
| Protease: Thrombin | DPRSFL |
| (SEQ ID NO: 30) | |
| Protease: Thrombin | PPRSFL |
| (SEQ ID NO: 31) | |
| Protease: Caspase-3 | DEVD |
| (SEQ ID NO: 32) | |
| Protease: Caspase-3 | DEVDP |
| (SEQ ID NO: 33) | |
| Protease: Caspase-3 | KGSGDVEG |
| (SEQ ID NO: 34) | |
| Protease: Interleukin | GWEHDG |
| 1ß converting enzyme | |
| (SEQ ID NO: 35) | |
| Protease: Enterokinase | EDDDDKA |
| (SEQ ID NO: 36) | |
| Protease: FAP | KQEQNPGST |
| (SEQ ID NO: 37) | |
| Protease: Kallikrein 2 | GKAFRR |
| (SEQ ID NO: 38) | |
| Protease: Plasmin | DAFK |
| (SEQ ID NO: 39) | |
| Protease: Plasmin | DVLK |
| (SEQ ID NO: 40) | |
| Protease: Plasmin | DAFK |
| (SEQ ID NO: 41) | |
| Protease: TOP | ALLLALL |
| (SEQ ID NO: 42) | |
| PSMA Prodrug C1872 | EVQLVESGGGLVQPGNSLRLSCAASGFTFSKFGMSWVRQGGG |
| (SEQ ID NO: 43) | GGLDGNEEPGGLEWVSSISGSGRDTLYADSVKGRFTISRDNA |
| | KTTLYLQMNSLRPEDTAVYYCTIGGSLSVSSQGTLVTVSSGG |
| | GGKPLGLQARVVGGGGT |
| | QTVVTQEPSLTVSPGGTVTLTCASSTGAVTSGNYPNWVQQKP |
| | GQAPRGLIGGTKFLVPGTPARFSGSLLGGKAALTLSGVQPED |
| | EAEYYCTLWYSNRWVFGGGTKLTVLGGGGSGGGGSGGGGSEV |
| | QLVESGGGLVQPGGSLKLSCAASGFTFNKYAINWVRQAPGKG |
| | LEWVARIRSKYNNYATYYADQVKDRFTISRDDSKNTAYLQMN |
| | NLKTEDTAVYYCVRHANFGNSYISYWAYWGQGTLVTVSSGGG |
| | GSGGGSEVQLVESGGGLVQPGGSLTLSCAASREMISEYHMHW |
| | VRQAPGKGLEWVSTINPAGTTDYAESVKGRFTISRDNAKNTL |
| | YLQMNSLKPEDTAVYYCDSYGYRGQGTQVTVSSHHHHHH |
| PSMA Non-cleavable | EVQLVESGGGLVQPGNSLRLSCAASGFTFSKFGMSWVRQGGG |
| Prodrug C1873 | GGLDGNEEPGGLEWVSSISGSGRDTLYADSVKGRFTISRDNA |
| (SEQ ID NO: 44) | KTTLYLQMNSLRPEDTAVYYCTIGGSLSVSSQGTLVTVSSGG |
| | GGSGGGGSGGVVGGGGT |
| | QTVVTQEPSLTVSPGGTVTLTCASSTGAVTSGNYPNWVQQKP |
| | GQAPRGLIGGTKFLVPGTPARFSGSLLGGKAALTLSGVQPED |
| | EAEYYCTLWYSNRWVFGGGTKLTVLGGGGSGGGGSGGGGSEV |
| | QLVESGGGLVQPGGSLKLSCAASGFTFNKYAINWVRQAPGKG |
| | LEWVARIRSKYNNYATYYADQVKDRFTISRDDSKNTAYLQMN |
| | NLKTEDTAVYYCVRHANFGNSYISYWAYWGQGTLVTVSSGGG |
| | GSGGGSEVQLVESGGGLVQPGGSLTLSCAASREMISEYHMHW |
| | VRQAPGKGLEWVSTINPAGTTDYAESVKGRFTISRDNAKNTL |
| | YLQMNSLKPEDTAVYYCDSYGYRGQGTQVTVSSHHHHHH |
| PSMA Active Drug C1875 | VVGGGGTQTVVTQEPSLTVSPGGTVTLTCASSTGAVTSGNYP |
| (SEQ ID NO: 45) | NWVQQKPGQAPRGLIGGTKFLVPGTPARFSGSLLGGKAALTL |
| | SGVQPEDEAEYYCTLWYSNRWVFGGGTKLTVLGGGGSGGGGS |
| | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSEVQLVESGGGLVQPGGSLTLSCAASRFMI |
| | SEYHMHWVRQAPGKGLEWVSTINPAGTTDYAESVKGRFTISR |
| | DNAKNTLYLQMNSLKPEDTAVYYCDSYGYRGQGTQVTVSSHH |
| | HHHH |
| EGFR (G8) Prodrug C1486 | EVQLVESGGGLVQPGNSLRLSCAASGFTFSKFGMSWVRQGGG |
| (SEQ ID NO: 46) | GGLDGNEEPGGLEWVSSISGSGRDTLYADSVKGRFTISRDNA |
| | KTTLYLQMNSLRPEDTAVYYCTIGGSLSVSSQGTLVTVSSGG |
| | GGKPLGLQARVVGGGGT |
| | QTVVTQEPSLTVSPGGTVTLTCASSTGAVTSGNYPNWVQQKP |
| | GQAPRGLIGGTKFLVPGTPARFSGSLLGGKAALTLSGVQPED |
| | EAEYYCTLWYSNRWVFGGGTKLTVLGGGGSGGGGSGGGGSEV |
| | QLVESGGGLVQPGGSLKLSCAASGFTFNKYAINWVRQAPGKG |
| | LEWVARIRSKYNNYATYYADQVKDRFTISRDDSKNTAYLQMN |
| | NLKTEDTAVYYCVRHANFGNSYISYWAYWGQGTLVTVSSGGG |
| | GSGGGSEVQLVESGGGLVQPGGSLTLSCAASGRTFSSYAMGW |
| | FRQAPGKEREFVVAINWASGSTYYADSVKGRFTISRDNSKNT |
| | LYLQMNSLRAEDTAVYYCAAGYQINSGNYNFKDYEYDYWGQG |
| | TLVTVSSHHHHHH |
| EGFR (G8) Non-cleavable | EVQLVESGGGLVQPGNSLRLSCAASGFTFSKFGMSWVRQGGG |
| Prodrug C1756 | GGLDGNEEPGGLEWVSSISGSGRDTLYADSVKGRFTISRDNA |
| (SEQ ID NO: 47) | KTTLYLQMNSLRPEDTAVYYCTIGGSLSVSSQGTLVTVSSGG |
| | GGSGGGGSGGVVGGGGT |
| | QTVVTQEPSLTVSPGGTVTLTCASSTGAVTSGNYPNWVQQKP |
| | GQAPRGLIGGTKFLVPGTPARFSGSLLGGKAALTLSGVQPED |
| | EAEYYCTLWYSNRWVFGGGTKLTVLGGGGSGGGGSGGGGSEV |
| | QLVESGGGLVQPGGSLKLSCAASGFTFNKYAINWVRQAPGKG |
| | LEWVARIRSKYNNYATYYADQVKDRFTISRDDSKNTAYLQMN |
| | NLKTEDTAVYYCVRHANFGNSYISYWAYWGQGTLVTVSSGGG |
| | GSGGGSEVQLVESGGGLVQPGGSLTLSCAASGRTFSSYAMGW |
| | FRQAPGKEREFVVAINWASGSTYYADSVKGRFTISRDNSKNT |
| | LYLQMNSLRAEDTAVYYCAAGYQINSGNYNFKDYEYDYWGQG |
| | TLVTVSSHHHHHH |
| EGFR (G8) Active Drug | VVGGGGTQTVVTQEPSLTVSPGGTVTLTCASSTGAVTSGNYP |
| C1300 (SEQ ID NO: 48) | NWVQQKPGQAPRGLIGGTKFLVPGTPARFSGSLLGGKAALTL |
| | SGVQPEDEAEYYCTLWYSNRWVFGGGTKLTVLGGGGSGGGGS |
| | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSEVQLVESGGGLVQPGGSLTLSCAASGRTF |
| | SSYAMGWFRQAPGKEREFVVAINWASGSTYYADSVKGRFTIS |
| | RDNSKNTLYLQMNSLRAEDTAVYYCAAGYQINSGNYNFKDYE |
| | YDYWGQGTLVTVSSHHHHHH |
| GFP TriTAC C646 | QVQLVESGGALVQPGGSLRLSCAASGFPVNRYSMRWYRQAPG |
| (SEQ ID NO: 49) | KEREWVAGMSSAGDRSSYEDSVKGRFTISRDDARNTVYLQMN |
| | SLKPEDTAVYYCNVNVGFEYWGQGTQVTVSSGGGGSGGGSEV |
| | QLVESGGGLVQPGNSLRLSCAASGFTFSKFGMSWVRQAPGKG |
| | LEWVSSISGSGRDTLYADSVKGRFTISRDNAKTTLYLQMNSL |
| | RPEDTAVYYCTIGGSLSVSSQGTLVTVSSGGGGSGGGSEVQL |
| | VESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSSGGGGS |
| | GGGGSGGGGSQTVVTQEPSLTVSPGGTVTLTCASSTGAVTSG |
| | NYPNWVQQKPGQAPRGLIGGTKFLVPGTPARFSGSLLGGKAA |
| | LTLSGVQPEDEAEYYCTLWYSNRWVFGGGTKLTVLHHHHHH |
| Exemplary masking | GGGGGLDGNEEPGG |
| sequence (SEQ ID NO: | |
| 50) | |
| Exemplary non-cleavable | GGGGSGGGGSGGVVGGGGT |
| linker sequence | |
| comprising protease | |
| cleavage site | |
| (SEQ ID NO: 51) | |
| PSMA Non-masked Non- | EVQLVESGGGLVQPGNSLRLSCAASGFTFSKFGMSWVRQAPG |
| cleavable Prodrug C1874 | KGLEWVSSISGSGRDTLYADSVKGRFTISRDNAKTTLYLQMN |
| (SEQ ID NO: 52) | SLRPEDTAVYYCTIGGSLSVSSQGTLVTVSSGGGGSGGGGSG |
| | GVVGGGGTQTVVTQEPSLTVSPGGTVTLTCASSTGAVTSGNY |
| | PNWVQQKPGQAPRGLIGGTKFLVPGTPARFSGSLLGGKAALT |
| | LSGVQPEDEAEYYCTLWYSNRWVFGGGTKLTVLGGGGSGGGG |
| | SGGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGSEVQLVESGGGLVQPGGSLTLSCAASRFM |
| | ISEYHMHWVRQAPGKGLEWVSTINPAGTTDYAESVKGRFTIS |
| | RDNAKNTLYLQMNSLKPEDTAVYYCDSYGYRGQGTQVTVSSH |
| | HHHHH |
| Exemplary cleavable | GGGGKPLGLQARVVGGGGT |
| linker | |
| (SEQ ID NO: 53) | |
| Exemplary anti-albumin | EVQLVESGGGLVQPGNSLRLSCAASGFTFSKFGMSWVRQGGG |
| sdAb | GGLDGNEEPGGLEWVSSISGSGRDTLYADSVKGRFTISRDNA |
| (SEQ ID NO: 54) | KTTLYLQMNSLRPEDTAVYYCTIGGSLSVSSQGTLVTVSSGG |
| | GGKPLGLQARVVGGGGT |
| Exemplary anti-target | EVQLVESGGGLVQPGGSLTLSCAASGRTFSSYAMGWFRQAPG |
| (anti-EGFR) sdAb | KEREFVVAINWASGSTYYADSVKGRFTISRDNSKNTLYLQMN |
| (SEQ ID NO: 55) | SLRAEDTAVYYCAAGYQINSGNYNFKDYEYDYWGQGTLVTVS |
| | S |
| Exemplary anti-CD3 scFv | QTVVTQEPSLTVSPGGTVTLTCASSTGAVTSGNYPNWVQQKP |
| (SEQ ID NO: 56) | GQAPRGLIGGTKFLVPGTPARFSGSLLGGKAALTLSGVQPED |
| | EAEYYCTLWYSNRWVFGGGTKLTVLGGGGSGGGGSGGGGSEV |
| | QLVESGGGLVQPGGSLKLSCAASGFTFNKYAINWVRQAPGKG |
| | LEWVARIRSKYNNYATYYADQVKDRFTISRDDSKNTAYLQMN |
| | NLKTEDTAVYYCVRHANFGNSYISYWAYWGQGTLVTVSS |
| Exemplary anti-target | EVQLVESGGGLVQPGGSLTLSCAASREMISEYHMHWVRQAPG |
| (anti-PSMA) sdAb | KGLEWVSTINPAGTTDYAESVKGRFTISRDNAKNTLYLQMNS |
| (SEQ ID NO: 57) | LKPEDTAVYYCDSYGYRGQGTQVTVSS |
| Exemplary anti-target | EVQLVESGGGLVQPGGSLRLSCAASREMISEYHMHWVRQAPG |
| (anti-PSMA) sdAb | KGLEWVSDINPAGTTDYAESVKGRFTISRDNAKNTLYLQMNS |
| (SEQ ID NO: 63) | LRAEDTAVYYCDSYGYRGQGTLVTVSS |
| Exemplary anti-target | EVQLVESGGGLVQPGGSLRLSCAASREMISEYHMHWVRQAPG |
| (anti-PSMA) sdAb | KGLEWVSTINPAGTTDYAESVKGRFTISRDNAKNTLYLQMNS |
| (SEQ ID NO: 64) | LRAEDTAVYYCDSYGYRGQGTLVTVSS |
| Exemplary anti-target | EVQLVESGGGLVQPGGSLRLSCAASREMISEYSMHWVRQAPG |
| (anti-PSMA) sdAb | KGLEWVSTINPAKTTDYAESVKGRFTISRDNAKNTLYLQMNS |
| (SEQ ID NO: 65) | LRAEDTAVYYCDSYGYRGQGTLVTVSS |
| Exemplary anti-target | EVQLVESGGGLVQPGGSLRLSCAASREMISPYSMHWVRQAPG |
| (anti-PSMA) sdAb | KGLEWVSTINPAGTTDYAESVKGRFTISRDNAKNTLYLQMNS |
| (SEQ ID NO: 66) | LRAEDTAVYYCDGYGYRGQGTLVTVSS |
| Exemplary anti-target | EVQLVESGGGLVQPGGSLRLSCAASREMISEYSMHWVRQAPG |
| (anti-PSMA) sdAb | KGLEWVSTINPAGQTDYAESVKGRFTISRDNAKNTLYLQMNS |
| (SEQ ID NO: 67) | LRAEDTAVYYCDGYGYRGQGTLVTVSS |
| Exemplary anti-target | EVQLVESGGGLVQPGGSLRLSCAASREMISEYSMHWVRQAPG |
| (anti-PSMA) sdAb | KGLEWVSTINPAGTTDYAEYVKGRFTISRDNAKNTLYLQMNS |
| (SEQ ID NO: 68) | LRAEDTAVYYCDGYGYRGQGTLVTVSS |
| Exemplary anti-target | EVQLVESGGGLVQPGGSLRLSCAASREMISEYHMHWVRQAPG |
| (anti-PSMA) sdAb | KGLEWVSDINPAKTTDYAESVKGRFTISRDNAKNTLYLQMNS |
| (SEQ ID NO: 69) | LRAEDTAVYYCDSYGYRGQGTLVTVSS |
| Exemplary anti-target | EVQLVESGGGLVQPGGSLRLSCAASREMISPYHMHWVRQAPG |
| (anti-PSMA) sdAb | KGLEWVSDINPAGTTDYAESVKGRFTISRDNAKNTLYLQMNS |
| (SEQ ID NO: 70) | LRAEDTAVYYCDSYGYRGQGTLVTVSS |
| Exemplary anti-target | EVQLVESGGGLVQPGGSLRLSCAASREMISEYHMHWVRQAPG |
| (anti-PSMA) sdAb | KGLEWVSDINPAGQTDYAESVKGRFTISRDNAKNTLYLQMNS |
| (SEQ ID NO: 71) | LRAEDTAVYYCDSYGYRGQGTLVTVSS |
| Exemplary anti-target | EVQLVESGGGLVQPGGSLRLSCAASREMISEYHMHWVRQAPG |
| (anti-PSMA) sdAb | KGLEWVSDINPAGTTDYAEYVKGRFTISRDNAKNTLYLQMNS |
| (SEQ ID NO: 72) | LRAEDTAVYYCDSYGYRGQGTLVTVSS |
| Exemplary anti-target | EVQLVESGGGLVQPGGSLTLSCAASREMISEYHMHWVRQAPG |
| (anti-PSMA) sdAb | KGLEWVSDINPAGTTDYAESVKGRFTISRDNAKNTLYLQMNS |
| (SEQ ID NO: 73) | LKPEDTAVYYCDSYGYRGQGTQVTVSS |
| Exemplary anti-CD3 scFv | EVQLVESGGGLVQPGGSLKLSCAASGFTFNKYAINWVRQAPG |
| (SEQ ID NO: 74) | KGLEWVARIRSKYNNYATYYADQVKDRFTISRDDSKNTAYLQ |
| | MNNLKTEDTAVYYCVRHANFGNSYISYWAYWGQGTLVTVSSG |
| | GGGSGGGGSGGGGSQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVL |
| Exemplary anti-CD3 scFv | EVQLVESGGGLVQPGGSLKLSCAASGFEFNKYAMNWVRQAPG |
| (SEQ ID NO: 75) | KGLEWVARIRSKYNKYATYYADSVKDRFTISRDDSKNTAYLQ |
| | MNNLKTEDTAVYYCVRHGNFGNSYISYWAYWGQGTLVTVSSG |
| | GGGSGGGGSGGGGSQTVVTQEPSLTVSPGGTVTLTCGSSFGA |
| | VTSGNYPNWVQQKPGQAPRGLIGGTKFLAPGTPARFSGSLLG |
| | GKAALTLSGVQPEDEAEYYCVLWYDNRWVFGGGTKLTVL |
| Exemplary anti-CD3 scFv | EVQLVESGGGLVQPGGSLKLSCAASGFTFNKYAMNWVRQAPG |
| (SEQ ID NO: 76) | KGLEWVARIRSKYNNYATYYADSVKDRFTISRDDSKNTAYLQ |
| | MNNLKTEDTAVYYCVRHGNFGNSHISYWAYWGQGTLVTVSSG |
| | GGGSGGGGSGGGGSQTVVTQEPSLTVSPGGTVTLTCGSSTGY |
| | VTSGNYPNWVQQKPGQAPRGLIGGTSFLAPGTPARFSGSLLG |
| | GKAALTLSGVQPEDEAEYYCVLWYSNRWIFGGGTKLTVL |
| Exemplary anti-CD3 ScFv | EVQLVESGGGLVQPGGSLKLSCAASGFMENKYAMNWVRQAPG |
| (SEQ ID NO: 77) | KGLEWVARIRSKSNNYATYYADSVKDRFTISRDDSKNTAYLQ |
| | MNNLKTEDTAVYYCVRHGNFGNSYISYWATWGQGTLVTVSSG |
| | GGGSGGGGSGGGGSQTVVTQEPSLTVSPGGTVTLTCGSSFGA |
| | VTSGNYPNWVQQKPGQAPRGLIGGTKLLAPGTPARFSGSLLG |
| | GKAALTLSGVQPEDEAEYYCVLWYSNSWVFGGGTKLTVL |
| Exemplary anti-CD3 scFv | EVQLVESGGGLVQPGGSLKLSCAASGFTENTYAMNWVRQAPG |
| (SEQ ID NO: 78) | KGLEWVARIRSKYNNYATYYKDSVKDRFTISRDDSKNTAYLQ |
| | MNNLKTEDTAVYYCVRHGNFGNSPISYWAYWGQGTLVTVSSG |
| | GGGSGGGGSGGGGSQTVVTQEPSLTVSPGGTVTLTCGSSTGA |
| | VVSGNYPNWVQQKPGQAPRGLIGGTEFLAPGTPARFSGSLLG |
| | GKAALTLSGVQPEDEAEYYCVLWYSNRWVFGGGTKLTVL |
| Exemplary anti-CD3 scFv | EVQLVESGGGLVQPGGSLKLSCAASGFTYNKYAMNWVRQAPG |
| (SEQ ID NO: 79) | KGLEWVARIRSKYNNYATYYADEVKDRFTISRDDSKNTAYLQ |
| | MNNLKTEDTAVYYCVRHGNFGNSPISYWAYWGQGTLVTVSSG |
| | GGGSGGGGSGGGGSQTVVTQEPSLTVSPGGTVTLTCGSSKGA |
| | VTSGNYPNWVQQKPGQAPRGLIGGTKELAPGTPARFSGSLLG |
| | GKAALTLSGVQPEDEAEYYCTLWYSNRWVFGGGTKLTVL |
| Exemplary anti-CD3 scFv | EVQLVESGGGLVQPGGSLKLSCAASGNTFNKYAMNWVRQAPG |
| (SEQ ID NO: 80) | KGLEWVARIRSKYNNYETYYADSVKDRFTISRDDSKNTAYLQ |
| | MNNLKTEDTAVYYCVRHTNFGNSYISYWAYWGQGTLVTVSSG |
| | GGGSGGGGSGGGGSQTVVTQEPSLTVSPGGTVTLTCGSSTGA |
| | VTSGYYPNWVQQKPGQAPRGLIGGTYFLAPGTPARFSGSLLG |
| | GKAALTLSGVQPEDEAEYYCVLWYSNRWVFGGGTKLTVL |
| Exemplary anti-CD3 scFv | EVQLVESGGGLVQPGGSLKLSCAASGFTENNYAMNWVRQAPG |
| (SEQ ID NO: 81) | KGLEWVARIRSKYNNYATYYADAVKDRFTISRDDSKNTAYLQ |
| | MNNLKTEDTAVYYCVRHGNFGNSQISYWAYWGQGTLVTVSSG |
| | GGGSGGGGSGGGGSQTVVTQEPSLTVSPGGTVTLTCGSSTGA |
| | VTDGNYPNWVQQKPGQAPRGLIGGIKFLAPGTPARFSGSLLG |
| | GKAALTLSGVQPEDEAEYYCVLWYSNRWVFGGGTKLTVL |
| Exemplary anti-CD3 scFv | EVQLVESGGGLVQPGGSLKLSCAASGFTFNKYAVNWVRQAPG |
| (SEQ ID NO: 82) | KGLEWVARIRSKYNNYATYYADSVKDRFTISRDDSKNTAYLQ |
| | MNNLKTEDTAVYYCVRHGNFGNSYISYWAYWGQGTLVTVSSG |
| | GGGSGGGGSGGGGSQTVVTQEPSLTVSPGGTVTLTCGESTGA |
| | VTSGNYPNWVQQKPGQAPRGLIGGTKILAPGTPARFSGSLLG |
| | GKAALTLSGVQPEDEAEYYCVLWYSNRWVFGGGTKLTVL |
| Exemplary anti-CD3 scFv | EVQLVESGGGLVQPGGSLKLSCAASGFTFNKYPMNWVRQAPG |
| (SEQ ID NO: 83) | KGLEWVARIRSKYNNYATYYADSVKDRFTISRDDSKNTAYLQ |
| | MNNLKNEDTAVYYCVRHGNENNSYISYWAYWGQGTLVTVSSG |
| | GGGSGGGGSGGGGSQTVVTQEPSLTVSPGGTVTLTCGSSTGA |
| | VTKGNYPNWVQQKPGQAPRGLIGGTKMLAPGTPARFSGSLLG |
| | GKAALTLSGVQPEDEAEYYCALWYSNRWVFGGGTKLTVL |
| Exemplary anti-CD3 scFv | EVQLVESGGGLVQPGGSLKLSCAASGFTENGYAMNWVRQAPG |
| (SEQ ID NO: 84) | KGLEWVARIRSKYNNYATYYADEVKDRFTISRDDSKNTAYLQ |
| | MNNLKTEDTAVYYCVRHGNFGNSPISYWAYWGQGTLVTVSSG |
| | GGGSGGGGSGGGGSQTVVTQEPSLTVSPGGTVTLTCGSSTGA |
| | VVSGNYPNWVQQKPGQAPRGLIGGTEFLAPGTPARFSGSLLG |
| | GKAALTLSGVQPEDEAEYYCVLWYSNRWVFGGGTKLTVL |
| Exemplary anti-CD3 scFv | EVQLVESGGGLVQPGGSLKLSCAASGNTFNKYAMNWVRQAPG |
| (SEQ ID NO: 85) | KGLEWVARIRSKYNNYATYYADSVKDRFTISRDDSKNTAYLQ |
| | MNNLKTEDTAVYYCVRHGNFGDSYISYWAYWGQGTLVTVSSG |
| | GGGSGGGGSGGGGSQTVVTQEPSLTVSPGGTVTLTCGSSTGA |
| | VTHGNYPNWVQQKPGQAPRGLIGGTKVLAPGTPARFSGSLLG |
| | GKAALTLSGVQPEDEAEYYCVLWYSNRWVFGGGTKLTVL |
| Exemplary anti-CD3 scFv | EVQLVESGGGLVQPGGSLKLSCAASGFTENNYAMNWVRQAPG |
| (SEQ ID NO: 86) | KGLEWVARIRSGYNNYATYYADSVKDRFTISRDDSKNTAYLQ |
| | MNNLKTEDTAVYYCVRHGNFGNSYISYWAYWGQGTLVTVSSG |
| | GGGSGGGGSGGGGSQTVVTQEPSLTVSPGGTVTLTCGSYTGA |
| | VTSGNYPNWVQQKPGQAPRGLIGGTKFNAPGTPARFSGSLLG |
| | GKAALTLSGVQPEDEAEYYCVLWYANRWVFGGGTKLTVL |
| Exemplary anti-CD3 scFv | EVQLVESGGGLVQPGGSLKLSCAASGFEFNKYAMNWVRQAPG |
| (SEQ ID NO: 87) | KGLEWVARIRSKYNNYETYYADSVKDRFTISRDDSKNTAYLQ |
| | MNNLKTEDTAVYYCVRHGNFGNSLISYWAYWGQGTLVTVSSG |
| | GGGSGGGGSGGGGSQTVVTQEPSLTVSPGGTVTLTCGSSSGA |
| | VTSGNYPNWVQQKPGQAPRGLIGGTKFGAPGTPARFSGSLLG |
| | GKAALTLSGVQPEDEAEYYCVLWYSNRWVFGGGTKLTVL |
| Exemplary anti-CD3 ScFv | EVQLVESGGGLVQPGGSLKLSCAASGFTFNKYAMNWVRQAPG |
| (SEQ ID NO: 88) | KGLEWVARIRSKYNNYATYYADSVKDRFTISRDDSKNTAYLQ |
| | MNNLKTEDTAVYYCVRHGNFGNSYISYWAYWGQGTLVTVSSG |
| | GGGSGGGGSGGGGSQTVVTQEPSLTVSPGGTVTLTCGSSTGA |
| | VTSGNYPNWVQQKPGQAPRGLIGGTKFLAPGTPARFSGSLLG |
| | GKAALTLSGVQPEDEAEYYCVLWYSNRWVFGGGTKLTVL |
| Exemplary anti-CD3 scFv | EVQLVESGGGLVQPGGSLKLSCAASGFTFNKYALNWVRQAPG |
| (SEQ ID NO: 89) | KGLEWVARIRSKYNNYATEYADSVKDRFTISRDDSKNTAYLQ |
| | MNNLKTEDTAVYYCVRHGNFGNSPISYWAYWGQGTLVTVSSG |
| | GGGSGGGGSGGGGSQTVVTQEPSLTVSPGGTVTLTCGSSTGA |
| | VTSGNYPNWVQQKPGQAPRGLIGGTNFLAPGTPERFSGSLLG |
| | GKAALTLSGVQPEDEAEYYCVLWYSNRWAFGGGTKLTVL |
| Exemplary anti-CD3 scFv | EVQLVESGGGLVQPGGSLKLSCAASGFTFNEYAMNWVRQAPG |
| (SEQ ID NO: 90) | KGLEWVARIRSKYNNYATYYADDVKDRFTISRDDSKNTAYLQ |
| | MNNLKTEDTAVYYCVRHGNFGNSGISYWAYWGQGTLVTVSSG |
| | GGGSGGGGSGGGGSQTVVTQEPSLTVSPGGTVTLTCGSSTGA |
| | VTVGNYPNWVQQKPGQAPRGLIGGTEFLAPGTPARFSGSLLG |
| | GKAALTLSGVQPEDEAEYYCVLWYSNRWVFGGGTKLTVL |
| Exemplary anti-target | EVQLVESGGGLVQPGRSLTLSCAASTNIFSISPMGWYRQAPG |
| (BCMA) | KQRELVAAIHGFSTLYADSVKGRFTISRDNAKNSIYLQMNSL |
| (SEQ ID NO: 91) | RPEDTALYYCNKVPWGDYHPRNVYWGQGTQVTVSS |
| Exemplary anti-target | EVQLVESGGGLVQPGRSLTLSCAASTDIFSISPMGWYRQAPG |
| (BCMA) | KQRELVAAIHGGSTLYADSVKGRFTISRDNAKNSIYLQMNSL |
| (SEQ ID NO: 92) | RPEDTALYYCNKVPWGDYHPRNVAWGQGTQVTVSS |
| Exemplary anti-target | EVQLVESGGGLVQPGRSLTLSCAASTNDFSISPMGWYRQAPG |
| (BCMA) | KQRELVAAIHGGSTLYADSVKGRFTISRDNAKNSIYLQMNSL |
| (SEQ ID NO: 93) | RPEDTALYYCNKVPWGDYHPRNVAWGQGTQVTVSS |
| Exemplary anti-target | EVQLVESGGGLVQPGRSLTLSCAASTNIFSKSPMGWYRQAPG |
| (BCMA) | KQRELVAAIHGKSTLYADSVKGRFTISRDNAKNSIYLQMNSL |
| (SEQ ID NO: 94) | RPEDTALYYCNKVPWGDYHPRNVVWGQGTQVTVSS |
| Exemplary anti-target | EVQLVESGGGLVQPGRSLTLSCAASTNDFSISPMGWYRQAPG |
| (BCMA) | KQRELVAAIHGKSTLYADSVKGRFTISRDNAKNSIYLQMNSL |
| (SEQ ID NO: 95) | RPEDTALYYCNKVPWGDYHPRNVKWGQGTQVTVSS |
| Exemplary anti-target | EVQLVESGGGLVQPGRSLTLSCAASTNQFSISPMGWYRQAPG |
| (BCMA) | KQRELVAAIHGKSTLYADSVKGRFTISRDNAKNSIYLQMNSL |
| (SEQ ID NO: 96) | RPEDTALYYCNKVPWGDYHPRNVVWGQGTQVTVSS |
| Exemplary anti-target | EVQLVESGGGLVQPGRSLTLSCAASTNIFSSSPMGWYRQAPG |
| (BCMA) | KQRELVAAINGFSTLYADSVKGRFTISRDNAKNSIYLQMNSL |
| (SEQ ID NO: 97) | RPEDTALYYCNKVPWGDYHPRNVHWGQGTQVTVSS |
| Exemplary anti-target | EVQLVESGGGLVQPGRSLTLSCAASTNIFSSSPMGWYRQAPG |
| (BCMA) | KQRELVAAIHGFSTLYADSVKGRFTISRDNAKNSIYLQMNSL |
| (SEQ ID NO: 98) | RPEDTALYYCNKVPWGDYHPRNVYWGQGTQVTVSS |
| Exemplary anti-target | EVQLVESGGGLVQPGRSLTLSCAASTNIFSTSPMGWYRQAPG |
| (BCMA) | KQRELVAAIHGFQTLYADSVKGRFTISRDNAKNSIYLQMNSL |
| (SEQ ID NO: 99) | RPEDTALYYCNKVPWGDYHPRNVVWGQGTQVTVSS |
| Exemplary anti-target | EVQLVESGGGLVQPGRSLTLSCAASTNIFSRSPMGWYRQAPG |
| (BCMA) | KQRELVAAIHGFETLYADSVKGRFTISRDNAKNSIYLQMNSL |
| (SEQ ID NO: 100) | RPEDTALYYCNKVPWGDYHPRNVLWGQGTQVTVSS |
| Exemplary anti-target | EVQLVESGGGLVQPGRSLTLSCAASTNIFSESPMGWYRQAPG |
| (BCMA) | KQRELVAAIHGFTTLYADSVKGRFTISRDNAKNSIYLQMNSL |
| (SEQ ID NO: 101) | RPEDTALYYCNKVPWGDYHPRNVTWGQGTQVTVSS |
| Exemplary anti-target | EVQLVESGGGLVQPGRSLTLSCAASTNIFSDSPMGWYRQAPG |
| (BCMA) | KQRELVAAIHGFSTLYADSVKGRFTISRDNAKNSIYLQMNSL |
| (SEQ ID NO: 102) | RPEDTALYYCNKVPWGDYHPRNVAWGQGTQVTVSS |
| Exemplary anti-target | EVQLVESGGGLVQPGRSLTLSCAASTNIFSNSPMGWYRQAPG |
| (BCMA) | KQRELVAAIHGGSTLYADSVKGRFTISRDNAKNSIYLQMNSL |
| (SEQ ID NO: 103) | RPEDTALYYCNKVPWGDYHPRNVHWGQGTQVTVSS |
| Exemplary anti-target | EVQLVESGGGLVQPGRSLTLSCAASTNIFSNSPMGWYRQAPG |
| (BCMA) | KQRELVAAIHGRSTLYADSVKGRFTISRDNAKNSIYLQMNSL |
| (SEQ ID NO: 104) | RPEDTALYYCNKVPWGDYHPRNVMWGQGTQVTVSS |
| Exemplary anti-target | EVQLVESGGGLVQPGRSLTLSCAASTNIFSNSPMGWYRQAPG |
| (BCMA) | KQRELVAAIHGPSTLYADSVKGRFTISRDNAKNSIYLQMNSL |
| (SEQ ID NO: 105) | RPEDTALYYCNKVPWGDYHPRNVTWGQGTQVTVSS |
| Exemplary anti-target | EVQLVESGGGLVQPGRSLTLSCAASTNIFSNSPMGWYRQAPG |
| (BCMA) | KQRELVAAIHGDSTLYADSVKGRFTISRDNAKNSIYLQMNSL |
| (SEQ ID NO: 106) | RPEDTALYYCNKVPWGDYHPRNVRWGQGTQVTVSS |
| Exemplary anti-target | EVQLVESGGGLVQPGRSLTLSCAASTNIFSRSPMGWYRQAPG |
| (BCMA) | KQRELVAAIHGDSTLYADSVKGRFTISRDNAKNSIYLQMNSL |
| (SEQ ID NO: 107) | RPEDTALYYCNKVPWGDYHPRNVTWGQGTQVTVSS |
| Exemplary anti-target | EVQLVESGGGLVQPGRSLTLSCAASTNIFSKSPMGWYRQAPG |
| (BCMA) | KQRELVAAIHGQSTLYADSVKGRFTISRDNAKNSIYLQMNSL |
| (SEQ ID NO: 108) | RPEDTALYYCNKVPWGDYHPRNVTWGQGTQVTVSS |
| Exemplary anti-target | EVQLVESGGGLVQPGRSLTLSCAASTNIFSSSPMGWYRQAPG |
| (BCMA) | KQRELVAAIHGHSTLYADSVKGRFTISRDNAKNSIYLQMNSL |
| (SEQ ID NO: 109) | RPEDTALYYCNKVPWGDYHPRNVTWGQGTQVTVSS |
| Exemplary anti-target | EVQLVESGGGLVQPGRSLTLSCAASTNIFSSSPMGWYRQAPG |
| (BCMA) | KQRELVAAIHGESTLYADSVKGRFTISRDNAKNSIYLQMNSL |
| (SEQ ID NO: 110) | RPEDTALYYCNKVPWGDYHPRKVYWGQGTQVTVSS |
| Exemplary anti-target | EVQLVESGGGLVQPGRSLTLSCAASTNIFSRSPMGWYRQAPG |
| (BCMA) | KQRELVAAIHGNSTLYADSVKGRFTISRDNAKNSIYLQMNSL |
| (SEQ ID NO: 111) | RPEDTALYYCNKVPWGIYHPRNVYWGQGTQVTVSS |
| Exemplary anti-target | EVQLVESGGGLVQPGRSLTLSCAASTNIFSESPMGWYRQAPG |
| (BCMA) | KQRELVAAIHGNSTLYADSVKGRFTISRDNAKNSIYLQMNSL |
| (SEQ ID NO: 112) | RPEDTALYYCNKVPWGTYHPRNVYWGQGTQVTVSS |
| Exemplary anti-target | EVQLVESGGGLVQPGRSLTLSCAASTNIFSVSPMGWYRQAPG |
| (BCMA) | KQRELVAAIHGNSTLYADSVKGRFTISRDNAKNSIYLQMNSL |
| (SEQ ID NO: 113) | RPEDTALYYCNKVPWGKYHPRNVYWGQGTQVTVSS |
| Exemplary anti-target | EVQLVESGGGLVQPGRSLTLSCAASTNIFSVSPMGWYRQAPG |
| (BCMA) | KQRELVAAIHGNSTLYADSVKGRFTISRDNAKNSIYLQMNSL |
| (SEQ ID NO: 114) | RPEDTALYYCNKVPWGDYHPRNVYWGQGTQVTVSS |
| Exemplary anti-target | EVQLVESGGGLVQPGRSLTLSCAASTNIFSKSPMGWYRQAPG |
| (BCMA) | KQRELVAAIHGNSTLYADSVKGRFTISRDNAKNSIYLQMNSL |
| (SEQ ID NO: 115) | RPEDTALYYCNKVPWGDYHPREVYWGQGTQVTVSS |
| Exemplary anti-target | EVQLVESGGGLVQPGRSLTLSCAASTNIFSDSPMGWYRQAPG |
| (BCMA) | KQRELVAAIHGTSTLYADSVKGRFTISRDNAKNSIYLQMNSL |
| (SEQ ID NO: 116) | RPEDTALYYCNKVPWGDYHPRNVYWGQGTQVTVSS |
| Exemplary anti-target | EVQLVESGGGLVQPGRSLTLSCAASTNIFSRSPMGWYRQAPG |
| (BCMA) | KQRELVAAIHGTSTLYADSVKGRFTISRDNAKNSIYLQMNSL |
| (SEQ ID NO: 117) | RPEDTALYYCNKVPWGKYHPRNVYWGQGTQVTVSS |
| Exemplary anti-target | EVQLVESGGGLVQPGRSLTLSCAASTNIFSHSPMGWYRQAPG |
| (BCMA) | KQRELVAAIHGTSTLYADSVKGRFTISRDNAKNSIYLQMNSL |
| (SEQ ID NO: 118) | RPEDTALYYCNKVPWGRYHPRNVYWGQGTQVTVSS |
| Exemplary anti-target | EVQLVESGGGLVQPGRSLTLSCAASTNIFSTSPMGWYRQAPG |
| (BCMA) | KQRELVAAIHGTSTLYADSVKGRFTISRDNAKNSIYLQMNSL |
| (SEQ ID NO: 119) | RPEDTALYYCNKVPWGDYHPGNVYWGQGTQVTVSS |
| Exemplary anti-target | EVQLVESGGGLVQPGRSLTLSCAASTNIFSTSPMGWYRQAPG |
| (BCMA) | KQRELVAAIHGTSTLYADSVKGRFTISRDNAKNSIYLQMNSL |
| (SEQ ID NO: 120) | RPEDTALYYCNKVPWGDYHPRNVQWGQGTQVTVSS |
| Exemplary anti-target | EVQLVESGGGLVQPGRSLTLSCAASTNIFSLSPMGWYRQAPG |
| (BCMA) | KQRELVAAIHGDSTLYADSVKGRFTISRDNAKNSIYLQMNSL |
| (SEQ ID NO: 121) | RPEDTALYYCNKVPWGDYHPRNVYWGQGTQVTVSS |
| Exemplary anti-target | EVQLVESGGGLVQPGRSLTLSCAASTNIFSISPGGWYRQAPG |
| (BCMA) | KQRELVAAIHGSSTLYADSVKGRFTISRDNAKNSIYLQMNSL |
| (SEQ ID NO: 122) | RPEDTALYYCNKVPWGDYHPRNVYWGQGTQVTVSS |
| Exemplary anti-target | EVQLVESGGGLVQPGRSLTLSCAASTNHFSISPMGWYRQAPG |
| (BCMA) | KQRELVAAIHGSSTLYADSVKGRFTISRDNAKNSIYLQMNSL |
| (SEQ ID NO: 123) | RPEDTALYYCNKVPWGDYHPRVVYWGQGTQVTVSS |
| Exemplary anti-target | EVQLVESGGGLVQPGRSLTLSCAASTNIFSASPMGWYRQAPG |
| (BCMA) | KQRELVAAIHGSSTLYADSVKGRFTISRDNAKNSIYLQMNSL |
| (SEQ ID NO: 124) | RPEDTALYYCNKVPWGDYHPRNVNWGQGTQVTVSS |
| Exemplary anti-target | EVQLVESGGGLVQPGRSLTLSCAASTNIFSASPMGWYRQAPG |
| (BCMA) | KQRELVAAIHGSSTLYADSVKGRFTISRDNAKNSIYLQMNSL |
| (SEQ ID NO: 125) | RPEDTALYYCNKVPWGRYHPRNVYWGQGTQVTVSS |
| Exemplary anti-target | EVQLVESGGGLVQPGRSLTLSCAASTNISSISPMGWYRQAPG |
| (BCMA) | KQRELVAAIHGTSTLYADSVKGRFTISRDNAKNSIYLQMNSL |
| (SEQ ID NO: 126) | RPEDTALYYCNKVPWGDYHPGNVYWGQGTQVTVSS |
| Exemplary anti-target | EVQLVESGGGLVQPGRSLTLSCAASTNIESISPMGWYRQAPG |
| (BCMA) | KQRELVAAIHGTSTLYADSVKGRFTISRDNAKNSIYLQMNSL |
| (SEQ ID NO: 127) | RPEDTALYYCNKVPWGDYHPGNVYWGQGTQVTVSS |
| Exemplary anti-target | EVQLVESGGGLVQPGRSLTLSCAASTNIFSISPYGWYRQAPG |
| (BCMA) | KQRELVAAIHGTSTLYADSVKGRFTISRDNAKNSIYLQMNSL |
| (SEQ ID NO: 128) | RPEDTALYYCNKVPWGDYHPGNVYWGQGTQVTVSS |
| Exemplary anti-target | EVQLVESGGGLVQPGRSLTLSCAASTNIASISPMGWYRQAPG |
| (BCMA) | KQRELVAAIHGTSTLYADSVKGRFTISRDNAKNSIYLQMNSL |
| (SEQ ID NO: 129) | RPEDTALYYCNKVPWGDYHPGNVYWGQGTQVTVSS |
| Exemplary anti-target | EVQLVESGGGLVQPGRSLTLSCAASTNIASISPMGWYRQAPG |
| (BCMA) | KQRELVAAIHGKSTLYADSVKGRFTISRDNAKNSIYLQMNSL |
| (SEQ ID NO: 130) | RPEDTALYYCNKVPWGDYHPGNVYWGQGTQVTVSS |
| Exemplary anti-target | EVQLVESGGGLVQPGRSLTLSCAASTNIASISPMGWYRQAPG |
| (BCMA) | KQRELVAAIHGSSTLYADSVKGRFTISRDNAKNSIYLQMNSL |
| (SEQ ID NO: 131) | RPEDTALYYCNKVPWGDYHPGNVYWGQGTQVTVSS |
| Exemplary anti-target | EVQLVESGGGLVQPGRSLTLSCAASTNITSISPMGWYRQAPG |
| (BCMA) | KQRELVAAIHGDSTLYADSVKGRFTISRDNAKNSIYLQMNSL |
| (SEQ ID NO: 132) | RPEDTALYYCNKVPWGDYHPGNVYWGQGTQVTVSS |
| Exemplary anti-target | EVQLVESGGGLVQPGRSLTLSCAASTNIMSISPMGWYRQAPG |
| (BCMA) | KQRELVAAIHGNSTLYADSVKGRFTISRDNAKNSIYLQMNSL |
| (SEQ ID NO: 133) | RPEDTALYYCNKVPWGDYHPGNVYWGQGTQVTVSS |
| Exemplary anti-target | EVQLVESGGGLVQPGRSLTLSCAASTNITSISPMGWYRQAPG |
| (BCMA) | KQRELVAAIHGNSTLYADSVKGRFTISRDNAKNSIYLQMNSL |
| (SEQ ID NO: 134) | RPEDTALYYCNKVPWGDYHPGNVYWGQGTQVTVSS |
| Exemplary anti-target | EVQLVESGGGLVQPGRSLTLSCAASTNIVSISPMGWYRQAPG |
| (BCMA) | KQRELVAAIHGHSTLYADSVKGRFTISRDNAKNSIYLQMNSL |
| (SEQ ID NO: 135) | RPEDTALYYCNKVPWGDYHPGNVYWGQGTQVTVSS |
| Exemplary anti-target | EVQLVESGGGLVQPGRSLTLSCAASTNIVSISPMGWYRQAPG |
| (BCMA) | KQRELVAAIHGKSTLYADSVKGRFTISRDNAKNSIYLQMNSL |
| (SEQ ID NO: 136) | RPEDTALYYCNKVPWGDYHPGNVYWGQGTQVTVSS |
| Exemplary anti-target | EVQLVESGGGLVQPGRSLTLSCAASTNVVSISPMGWYRQAPG |
| (BCMA) | KQRELVAAIHGKSTLYADSVKGRFTISRDNAKNSIYLQMNSL |
| (SEQ ID NO: 137) | RPEDTALYYCNKVPWGDYHPNNVYWGQGTQVTVSS |
| Exemplary anti-target | EVQLVESGGGLVQPGRSLTLSCAASTNIISISPMGWYRQAPG |
| (BCMA) | KQRELVAAIHGASTLYADSVKGRFTISRDNAKNSIYLQMNSL |
| (SEQ ID NO: 138) | RPEDTALYYCNKVPWGDYHPGNVYWGQGTQVTVSS |
| Exemplary anti-target | EVQLVESGGGLVQPGRSLTLSCAASTNIFSITPMGWYRQAPG |
| (BCMA) | KQRELVAAIHGASTLYADSVKGRFTISRDNAKNSIYLQMNSL |
| (SEQ ID NO: 139) | RPEDTALYYCNKVPWGDYHPGNVYWGQGTQVTVSS |
| Exemplary anti-target | EVQLVESGGGLVQPGRSLTLSCAASTNITSISPMGWYRQAPG |
| (BCMA) | KQRELVAAIHGFETLYADSVKGRFTISRDNAKNSIYLQMNSL |
| (SEQ ID NO: 140) | RPEDTALYYCNKVPWGDYHPGNVYWGQGTQVTVSS |
| Exemplary anti-target | EVQLVESGGGLVQPGRSLTLSCAASTNIQSISPMGWYRQAPG |
| (BCMA) | KQRELVAAIHGFETLYADSVKGRFTISRDNAKNSIYLQMNSL |
| (SEQ ID NO: 141) | RPEDTALYYCNKVPWGDYHPGNVYWGQGTQVTVSS |
| Exemplary anti-target | EVQLVESGGGLVQPGRSLTLSCAASTSDFSISPMGWYRQAPG |
| (BCMA) | KQRELVAAIHGFETLYADSVKGRFTISRDNAKNSIYLQMNSL |
| (SEQ ID NO: 142) | RPEDTALYYCNKVPWGDYHPGNVYWGQGTQVTVSS |
| Exemplary anti-target | EVQLVESGGGLVQPGRSLTLSCAASTNIDSISPMGWYRQAPG |
| (BCMA) | KQRELVAAIHGFQTLYADSVKGRFTISRDNAKNSIYLQMNSL |
| (SEQ ID NO: 143) | RPEDTALYYCNKVPWGDYHPGNVYWGQGTQVTVSS |
| Exemplary anti-target | EVQLVESGGGLVQPGRSLTLSCAASTNIMSISPMGWYRQAPG |
| (BCMA) | KQRELVAAIHGFSTVYADSVKGRFTISRDNAKNSIYLQMNSL |
| (SEQ ID NO: 144) | RPEDTALYYCNKVPWGDYHPGNVYWGQGTQVTVSS |
| Exemplary anti-target | EVQLVESGGGLVQPGRSLTLSCAASTNIESISPMGWYRQAPG |
| (BCMA) | KQRELVAAIHGFSTLYADSVKGRFTISRDNAKNSIYLQMNSL |
| (SEQ ID NO: 145) | RPEDTALYYCNKVPWGDYHPGNVYWGQGTQVTVSS |
| Exemplary anti-target | EVQLVESGGGLVQPGRSLTLSCAASTNIFSSSPMGWYRQAPG |
| (BCMA) | KQRELVAAIHGFKTLYADSVKGRFTISRDNAKNSIYLQMNSL |
| (SEQ ID NO: 146) | RPEDTARYYCNKVPWGDYHPGNVYWGQGTQVTVSS |
| Exemplary anti-target | EVQLVESGGGLVQPGRSLTLSCAASTNIFSSSPMGWYRQAPG |
| (BCMA) | KQRELVAAIHGFSTLYADSVKGRFTISRDNAKNSIYLQMNSL |
| (SEQ ID NO: 147) | RPEDTALYYCNKVPWGDYHPGNVYWGQGTQVTVSS |
| Exemplary anti-target | EVQLVESGGGLVQPGRSLTLSCAASTNIFSNSPMGWYRQAPG |
| (BCMA) | KQRELVAAIHGFSTLYADSVKGRFTISRDNAKNSIYLQMNSL |
| (SEQ ID NO: 148) | RPEDTALYYCNKVPWGDYHPGNVYWGQGTQVTVSS |
| Exemplary anti-target | EVQLVESGGGLVQPGRSLTLSCAASTNIFSTSPMGWYRQAPG |
| (BCMA) | KQRELVAAIHGFSTIYADSVKGRFTISRDNAKNSIYLQMNSL |
| (SEQ ID NO: 149) | RPEDTALYYCNKVPWGDYHPGNVYWGQGTQVTVSS |
| Exemplary anti-target | EVQLVESGGGLVQPGRSLTLSCAASTNIFSTSPMGWYRQAPG |
| (BCMA) | KQRELVAAIHGFSTIYADSVKGRFTISRDNAKNSIYLQMNSL |
| (SEQ ID NO: 150) | RPEDTALYYCNKVPWGDYHPLNVYWGQGTQVTVSS |
| Exemplary anti-target | EVQLVESGGGLVQPGRSLTLSCVASTNIFSTSPMGWYRQAPG |
| (BCMA) | KQRELVAAIHGFSTLYADSVKGRFTISRDNAKNSIYLQMNSL |
| (SEQ ID NO: 151) | RPEDTALYYCNKVPWGDYHPGNVYWGQGTQVTVSS |
| Exemplary anti-target | EVQLVESGGGLVQPGRSLTLSCAASTNIFSDSPMGWYRQAPG |
| (BCMA) | KQRELVAAIHGFSTFYADSVKGRFTISRDNAKNSIYLQMNSL |
| (SEQ ID NO: 152) | RPEDTALYYCNKVPWGDYHPGNVYWGQGTQVTVSS |
| Exemplary anti-target | EVQLVESGGGLVQPGRSLTLSCAASTNIFSQSPMGWYRQAPG |
| (BCMA) | KQRELVAAIHGDSTLYADSVKGRFTISRDNAKNSIYLQMNSL |
| (SEQ ID NO: 153) | RPEDTALYYCNKVPWGDYHPGNVCWGQGTQVTVSS |
| Exemplary anti-target | EVQLVESGGGLVQPGRSLTLSCAASTNIFSQSPMGWYRQAPG |
| (BCMA) | KQRELVAAIHGKSTLYADSVKGRFTISRDNAKNSIYLQMNSL |
| (SEQ ID NO: 154) | RPEDTALYYCNKVPWGDYHPSNVYWGKGTQVTVSS |
| Exemplary anti-target | EVQLVESGGGLVQPGRSLTLSCAASTNIFSRSPMGWYRQAPG |
| (BCMA) | KQRELVAAIHGESTLYADSVKGRFTISRDNAKNSIYLQMNSL |
| (SEQ ID NO: 155) | RPEDTALYYCNKVPWGRYHPGNVYWGQGTQVTVSS |
| Exemplary anti-target | EVQLVESGGGLVQPGRSLTLSCAASTNIFSRSPMGWYRQAPG |
| (BCMA) | KQRELVAAIHGISTLYADSVKGRFTISRDNAKNSIYLQMNSL |
| (SEQ ID NO: 156) | RPEDTALYYCNKVPWGDYHPGNVYWGQGTQVTVSS |
| Exemplary anti-target | EVQLVESGGGLVQPGRSLTLSCAASTNIFSRSPMGWYRQAPG |
| (BCMA) | KQRELVAAIHGSSTLYADSVKGRFTISRDNAKNSIYLQMNSL |
| (SEQ ID NO: 157) | RPEDTALYYCNKVPWGDYHPGNVYWGQGTQVTVSS |
| Exemplary anti-target | EVQLVESGGGLVQPGRSLTLSCAASTNIFSGSPMGWYRQAPG |
| (BCMA) | KQRELVAAIHGNSTLYADSVKGRFTISRDNAKNSIYLQMNSL |
| (SEQ ID NO: 158) | RPEDTALYYCNKVPWGDYHPGNVYWGQGTQVTVSS |
| Exemplary anti-target | EVQLVESGGGLVQPGRSLTLSCAASSNIFSISPMGWYRQAPG |
| (BCMA) | KQRELVAAIHGSSTLYADSVKGRFTISRDNAKNSIYLQMNSL |
| (SEQ ID NO: 159) | RPEDTALYYCNKVPWGDYHPGNVYWGQGTQVTVSS |
| Exemplary anti-target | EVQLVESGGGLVQPGRSLTLSCAASTNIFSIYPMGWYRQAPG |
| (BCMA) | KQRELVAAIHGSSTLYADSVKGRFTISRDNAKNSIYLQMNSL |
| (SEQ ID NO: 160) | RPEDTALYYCNKVPWGDYHPKNVYWGQGTQVTVSS |
| Exemplary anti-target | EVQLVESGGGLVQPGRSLTLSCAASTNIFSKSPMGWYRQAPG |
| (BCMA) | KQRELVAAIHGSSTLYADSVKGRFTISRDNAKNSIYLQMNSL |
| (SEQ ID NO: 161) | RPEDTALYYCNKVPWGDYHPGNVYWGQGTQVTVSS |
| Exemplary anti-target | EVQLVESGGGLVQPGRSLTLSCAASTNIFSKSPMGWYRQAPG |
| (BCMA) | KQRELVAAIHGSSTLYADSVKGRFTISRDNAKNSIYLQMNSL |
| (SEQ ID NO: 162) | RPEDTALYYCNKVPWGDYHPRNVYWGQGTQVTVSS |
| Exemplary anti-target | EVQLVESGGGLVQPGRSLTLSCAASTNEFSISPMGWYRQAPG |
| (BCMA) | KQRELVAAIHGLSTLYADSVKGRFTISRDNAKNSIYLQMNSL |
| (SEQ ID NO: 163) | RPEDTALYYCNKVPWGAYHPRNVYWGQGTQVTVSS |
| Exemplary anti-target | EVQLVESGGGLVQPGRSLTLSCAASTNEFSISPMGWYRQAPG |
| (BCMA) | KQRELVAAIHGESTLYADSVKGRFTISRDNAKNSIYLQMNSL |
| (SEQ ID NO: 164) | RPEDTALYYCNKVPWGDYHPGNVYWGQGTQVTVSS |
| Exemplary anti-target | EVQLVESGGGLVQPGRSLTLSCAASTNIPSISPMGWYRQAPG |
| (BCMA) | KQRELVAAIHGESTLYADSVKGRFTISRDNAKNSIYLQMNSL |
| (SEQ ID NO: 165) | RPEDTALYYCNKVPWGDYHPRNVAWGQGTQVTVSS |
| Exemplary anti-target | EVQLVESGGGLVQPGRSLTLSCAASTNIPSISPMGWYRQAPG |
| (BCMA) | KQRELVAAIHGASTLYADSVKGRFTISRDNAKNSIYLQMNSL |
| (SEQ ID NO: 166) | RPEDTALYYCNKVPWGDYHPRNVAWGQGTQVTVSS |
| Exemplary anti-target | EVQLVESGGGLVQPGRSLTLSCAASTNIPSISPMGWYRQAPG |
| (BCMA) | KQRELVAAIHGESTLYADSVKGRFTISRDNAKNSIYLQMNSL |
| (SEQ ID NO: 167) | RPEDTALYYCNKVPWGDYHPRNVYWGQGTQVTVSS |
| Exemplary anti-target | EVQLVESGGGLVQPGRSLTLSCAASTNIPSISPMGWYRQAPG |
| (BCMA) | KQRELVAAIHGDSTLYADSVKGRFTISRDNAKNSIYLQMNSL |
| (SEQ ID NO: 168) | RPEDTALYYCNKVPWGDYHPRNVYWGQGTQVTVSS |
| Exemplary anti-target | EVQLVESGGGLVQPGRSLTLSCAASTNITSISPMGWYRQAPG |
| (BCMA) | KQRELVAAIHGVSTLYADSVKGRFTISRDNAKNSIYLQMNSL |
| (SEQ ID NO: 169) | RPEDTALYYCNKVPWGDYHPRNVQWGQGTQVTVSS |
| Exemplary anti-target | EVQLVESGGGLVQPGRSLTLSCAASTNIPSISPMGWYRQAPG |
| (BCMA) | KQRELVAAIHGQSTLYADSVKGRFTISRDNAKNSIYLQMNSL |
| (SEQ ID NO: 170) | RPEDTALYYCNKVPWGDYHPRNVQWGQGTQVTVSS |
| Exemplary anti-target | EVQLVESGGGLVQPGRSLTLSCAASTNIVSISPMGWYRQAPG |
| (BCMA) | KQRELVAAIHGDSTLYADSVKGRFTISRDNAKNSIYLQMNSL |
| (SEQ ID NO: 171) | RPEDTALYYCNKVPWGDYHPRNVSWGQGTQVTVSS |
| Exemplary anti-target | EVQLVESGGGLVQPGRSLTLSCAASSNIFSISPMGWYRQAPG |
| (BCMA) | KQRELVAAIHGESTLYADSVKGRFTISRDNAKNSIYLQMNSL |
| (SEQ ID NO: 172) | RPEDTALYYCNKVPWGDYHPRNVTWGQGTQVTVSS |
| Exemplary anti-target | EVQLVESGGGLVQPGRSLTLSCAASTNIDSISPMGWYRQAPG |
| (BCMA) | KQRELVAAIHGESTLYADSVKGRFTISRDNAKNSIYLQMNSL |
| (SEQ ID NO: 173) | RPEDTALYYCNKVPWGDYHPRNVTWGQGTQVTVSS |
| Exemplary anti-target | EVQLVESGGGLVQPGRSLTLSCAASTNIDSISPMGWYRQAPG |
| (BCMA) | KQRELVAAIHGSSTLYADSVKGRFTISRDNAKNSIYLQMNSL |
| (SEQ ID NO: 174) | RPEDTALYYCNKVPWGDYHPRNVTWGQGTQVTVSS |
| Exemplary anti-target | EVQLVESGGGLVQPGRSLTLSCAASTNIRSISPMGWYRQAPG |
| (BCMA) | KQRELVAAIHGSSTLYADSVKGRFTISRDNAKNSIYLQMNSL |
| (SEQ ID NO: 175) | RPEDTALYYCNKVPWGDYHPRNVVWGQGTQVTVSS |
| Exemplary anti-target | EVQLVESGGGLVQPGRSLTLSCAASTNITSISPMGWYRQAPG |
| (BCMA) | KQRELVAAISGFSTLYADSVKGRFTISRDNAKNSIYLQMNSL |
| (SEQ ID NO: 176) | RPEDTALYYCNEVPWGDYHPRNVYWGQGTQVTVSS |
| Exemplary anti-target | EVQLVESGGGLVQPGRSLTLSCAASTNITSISPMGWYRQAPG |
| (BCMA) | KQRELVAAIHGESTLYADSVKGRFTISRDNAKNSIYLQMNSL |
| (SEQ ID NO: 177) | RPEDTALYYCNKVPWGDYHPRNVYWGQGTQVTVSS |
| Exemplary anti-target | EVQLVESGGGLVQPGRSLTLSCAASTNITSVSPMGWYRQAPG |
| (BCMA) | KQRELVAAIHGPSTLYADSVKGRFTISRDNAKNSIYLQMNSL |
| (SEQ ID NO: 178) | RPEDTALYYCNKVPWGDYHPTNVYWGQGTQVTVSS |
| Exemplary anti-target | EVQLVESGGGLVQPGRSLTLSCAASTNIGSISPMGWYRQAPG |
| (BCMA) | KQRELVAAIHGQSTLYADSVKGRFTISRDNAKNSIYLQMNSL |
| (SEQ ID NO: 179) | RPEDTALYYCNKVPWGDYHPQNVYWGQGTQVTVSS |
| Exemplary anti-target | EVQLVESGGGLVQPGRSLTLSCAASTNIESISPMGWYRQAPG |
| (BCMA) | KQRELVAAIHGKSTLYADSVKGRFTISRDNAKNSIYLQMNSL |
| (SEQ ID NO: 180) | RPEDTALYYCNKVPWGDYHPRRVYWGQGTQVTVSS |
| Exemplary anti-target | EVQLVESGGGLVQPGRSLTLSCAASTNIVSISPMGWYRQAPG |
| (BCMA) | KQRELVAAIHGDSTLYADSVKGRFTISRDNAKNSIYLQMNSL |
| (SEQ ID NO: 189) | RPEDTALYYCNKVPWGDYHPRRVYWGQGTQVTVSS |
| Exemplary anti-target | EVQLVESGGGLVQPGRSLTLSCAASTNIDSISPMGWYRQAPG |
| (BCMA) | KQRELVAAIHGNSTLYADSVKGRFTISRDNAKNSIYLQMNSL |
| (SEQ ID NO: 190) | RPEDTALYYCNKVPWGDYHPRMVYWGQGTQVTVSS |
| Exemplary anti-target | EVQLVESGGGLVQPGRSLTLSCAASTNIFMISPMGWYRQAPG |
| (BCMA) | KQRELVAAIHGDSTLYADSVKGRFTISRDNAKNSIYLQMNSL |
| (SEQ ID NO: 191) | RPEDTALYYCNKVPWGRYHPRNVYWGQGTQVTVSS |
| Exemplary anti-target | EVQLVESGGGLVQPGRSLTLSCAASTNIFRISPMGWYRQAPG |
| (BCMA) | KQRELVAAIHGDSTLYADSVKGRFTISRDNAKNSIYLQMNSL |
| (SEQ ID NO: 192) | RPEDTALYYCNKVPWGRYHPRNVYWGQGTQVTVSS |
| Exemplary anti-target | EVQLVESGGGLVQPGRSLTLSCAASTNIFSISPMGWYRQAPG |
| (BCMA) | KQRELVAAIHGDSTLYADSVKGRFTISRDNAKNSIYLQMNSL |
| (SEQ ID NO: 193) | RPEDTALYYCNKVPWGEYHPRNVYWGQGTQVTVSS |
| Exemplary anti-target | EVQLVESGGGLVQPGRSLTLSCAASTNIFSISPMGWYRQAPG |
| (BCMA) | KQRELVAAIHGDSTLYADSVKGRFTISRDNAKNSIYLQMNSL |
| (SEQ ID NO: 194) | RPEDTALYYCNKVPWGKYHPRNVYWGQGTQVTVSS |
| Exemplary anti-target | EVQLVESGGGLVQPGRSLTLSCAASTNIESISPMGWYRQAPG |
| (BCMA) | KQRELVAAIHGSSTLYADSVKGRFTISRDNAKNSIYLQMNSL |
| (SEQ ID NO: 195) | RPEDTALYYCNKVPWGRYHPRNVYWGQGTQVTVSS |
| Exemplary anti-target | EVQLVESGGGLVQPGRSLTLSCAASTNIESISPMGWYRQAPG |
| (BCMA) | KQRELVAAIHGNSTLYADSVKGRFTISRDNAKNSIYLQMNSL |
| (SEQ ID NO: 196) | RPEDTALYYCNKVPWGRYHPRNVYWGQGTQVTVSS |
| Exemplary anti-target | EVQLVESGGGLVQPGRSLTLSCAASTNISSISPMGWYRQAPG |
| (BCMA) | KQRELVAAIHGFSTLYADSVKGRFTISRDNAKNSIYLQMNSL |
| (SEQ ID NO: 197) | RPEDTALYYCNKVPWGYYHPRNVYWGQGTQVTVSS |
| Exemplary anti-target | EVQLVESGGGLVQPGRSLTLSCAASTNISSISPMGWYRQAPG |
| (BCMA) | KQRELVAAIHGHSTLYADSVKGRFTISRDNAKNSIYLQMNSL |
| (SEQ ID NO: 198) | RPEDTALYYCNKVPWGRYHPRNVYWGQGTQVTVSS |
| Exemplary anti-target | EVQLVESGGGLVQPGRSLTLSCAASTNISSISPMGWYRQAPG |
| (BCMA) | KQRELVAAIHGFSTVYADSVKGRFTISRDNAKNSIYLQMNSL |
| (SEQ ID NO: 199) | RPEDTALYYCNKVPWGRYHPRNVYWGQGTQVTVSS |
| Exemplary anti-target | EVQLVESGGGLVQPGRSLTLSCAASTNIFSIRPMGWYRQAPG |
| (BCMA) | KQRELVAAIHGFSTVYADSVKGRFTISRDNAKNSIYLQMNSL |
| (SEQ ID NO: 200) | RPEDTALYYCNKVPWGDYHPRNVYWGQGTQVTVSS |
| Exemplary anti-target | EVQLVESGGGLVQPGRSLTLSCAASTNIFSIYPMGWYRQAPG |
| (BCMA) | KQRELVAAIHGFSTYYADSVKGRFTISRDNAKNSIYLQMNSL |
| (SEQ ID NO: 201) | RPEDTALYYCNKVPWGSYHPRNVYWGQGTQVTVSS |
| Exemplary anti-target | EVQLVESGGGLVQPGRSLTLSCAASTNIFNISPMGWYRQAPG |
| (BCMA) | KQRELVAAIHGFSTYYADSVKGRFTISRDNAKNSIYLQMNSL |
| (SEQ ID NO: 202) | RPEDTALYYCNKVPWGRYHPRNVYWGQGTQVTVSS |
| Exemplary anti-target | EVQLVESGGGLVQPGRSLTLSCAASTNIFSSSPMGWYRQAPG |
| (BCMA) | KQRELVAAIHGFSTWYADSVKGRFTISRDNAKNSIYLQMNSL |
| (SEQ ID NO: 203) | RPEDTALYYCNKVPWGRYHPRNVYWGQGTQVTVSS |
| Exemplary anti-target | EVQLVESGGGLVQPGRSLTLSCAASTNISSISPMGWYRQAPG |
| (BCMA) | KQRELVAAIHGFDTLYADSVKGRFTISRDNAKNSIYLQMNSL |
| (SEQ ID NO: 204) | RPEDTALYYCNKVPWGDYHPRNVYWGQGTQVTVSS |
| Exemplary anti-target | EVQLVESGGGLVQPGRSLTLSCAASTNIFSINPMGWYRQAPG |
| (BCMA) | KQRELVAAIHGFDTLYADSVKGRFTISRDNAKNSIYLQMNSL |
| (SEQ ID NO: 205) | RPEDTALYYCNKVPWGDYHPRNVSWGQGTQVTVSS |
| Exemplary anti-target | EVQLVESGGGLVQPGRSLTLSCAASTNIFSITPMGWYRQAPG |
| (BCMA) | KQRELVAAIHGRSTLYADSVKGRFTISRDNAKNSIYLQMNSL |
| (SEQ ID NO: 206) | RPEDTALYYCNKVPWGSYHPRNVYWGQGTQVTVSS |
| Exemplary anti-target | EVQLVESGGGLVQPGRSLTLSCAASTNIFSITPMGWYRQAPG |
| (BCMA) | KQRELVAAIHGTSTLYADSVKGRFTISRDNAKNSIYLQMNSL |
| (SEQ ID NO: 207) | RPEDTALYYCNKVPWGRYHPRNVYWGQGTQVTVSS |
| Exemplary anti-target | EVQLVESGGGLVQPGRSLTLSCAASTNIFSITPMGWYRQAPG |
| (BCMA) | KQRELVAAIHGESTLYADSVKGRFTISRDNAKNSIYLQMNSL |
| (SEQ ID NO: 208) | RPEDTALYYCNKVPWGRYHPRNVYWGQGTQVTVSS |
| Exemplary anti-target | EVQLVESGGGLVQPGRSLTLSCAASTNIFSITPMGWYRQAPG |
| (BCMA) | KQRELVAAIHGESTLYADSVKGRFTISRDNAKNSIYLQMNSL |
| (SEQ ID NO: 209) | RPEDTALYYCNKVPWGDYHPRDVYWGQGTQVTVSS |
| Exemplary anti-target | EVQLVESGGGLVQPGRSLTLSCAASTNIFSTSPYGWYRQAPG |
| (BCMA) | KQRELVAAIHGFSTIYADSVKGRFTISRDNAKNSIYLQMNSL |
| (SEQ ID NO: 210) | RPEDTALYYCNKVPWGDYHPGNVYWGQGTQVTVSS |
| Exemplary anti-target | EVQLVESGGGLVQPGRSLTLSCAASTNIFSTSPGGWYRQAPG |
| (BCMA) | KQRELVAAIHGFSTIYADSVKGRFTISRDNAKNSIYLQMNSL |
| (SEQ ID NO: 211) | RPEDTALYYCNKVPWGDYHPGNVYWGQGTQVTVSS |
| Exemplary anti-target | EVQLVESGGGLVQPGRSLTLSCAASTNIFSITPYGWYRQAPG |
| (BCMA) | KQRELVAAIHGASTLYADSVKGRFTISRDNAKNSIYLQMNSL |
| (SEQ ID NO: 212) | RPEDTALYYCNKVPWGDYHPGNVYWGQGTQVTVSS |
| Exemplary anti-target | EVQLVESGGGLVQPGRSLTLSCAASTNIFSITPGGWYRQAPG |
| (BCMA) | KQRELVAAIHGASTLYADSVKGRFTISRDNAKNSIYLQMNSL |
| (SEQ ID NO: 213) | RPEDTALYYCNKVPWGDYHPGNVYWGQGTQVTVSS |
| Exemplary anti-target | QVQLVESGGGLVQPGESLRLSCAASTNIFSISPMGWYRQAPG |
| (BCMA) | KQRELVAAIHGFSTLYADSVKGRFTISRDNAKNTIYLQMNSL |
| (SEQ ID NO: 214) | KPEDTAVYYCNKVPWGDYHPRNVYWGQGTQVTVSS |
| Exemplary anti-target | QVQLVESGGGLVQPGGSLRLSCAASGRTFSVRGMAWYRQAGN |
| (MSLN) | NRALVATMNPDGFPNYADAVKGRFTISWDIAENTVYLQMNSL |
| (SEQ ID NO: 215) | NSEDTTVYYCNSGPYWGQGTQVTVSS |
| Exemplary anti-target | QVQLVESGGGLVQAGGSLRLSCAASGSIPSIEQMGWYRQAPG |
| (MSLN) | KQRELVAALTSGGRANYADSVKGRFTISGDNVRNMVYLQMNS |
| (SEQ ID NO: 216) | LKPEDTAIYYCSAGRFKGDYAQRSGMDYWGKGTLVTVSS |
| Exemplary anti-target | QVQLVESGGGLVQAGGSLRLSCAFSGTTYTFDLMSWYRQAPG |
| (MSLN) | KQRTVVASISSDGRTSYADSVRGRFTISGENGKNTVYLQMNS |
| (SEQ ID NO: 217) | LKLEDTAVYYCLGQRSGVRAFWGQGTQVTVSS |
| Exemplary anti-target | QVQLVESGGGLVQAGGSLRLSCVASGSTSNINNMRWYRQAPG |
| (MSLN) | KERELVAVITRGGYAIYLDAVKGRFTISRDNANNAIYLEMNS |
| (SEQ ID NO: 218) | LKPEDTAVYVCNADRVEGTSGGPQLRDYFGQGTQVTVSS |
| Exemplary anti-target | QVQLVESGGGLVQAGGSLRLSCAASGSTFGINAMGWYRQAPG |
| (MSLN) | KQRELVAVISRGGSTNYADSVKGRFTISRDNAENTVSLQMNT |
| (SEQ ID NO: 219) | LKPEDTAVYFCNARTYTRHDYWGQGTQVTVSS |
| Exemplary anti-target | QVRLVESGGGLVQAGGSLRLSCAASISAFRLMSVRWYRQDPS |
| (MSLN) | KQREWVATIDQLGRTNYADSVKGRFAISKDSTRNTVYLQMNM |
| (SEQ ID NO: 220) | LRPEDTAVYYCNAGGGPLGSRWLRGRHWGQGTQVTVSS |
| Exemplary anti-target | QVRLVESGGGLVQAGESLRLSCAASGRPFSINTMGWYRQAPG |
| (MSLN) | KQRELVASISSSGDFTYTDSVKGRFTISRDNAKNTVYLQMNS |
| (SEQ ID NO: 221) | LKPEDTAVYYCNARRTYLPRRFGSWGQGTQVTVSS |
| Exemplary anti-target | QVQPVESGGGLVQPGGSLRLSCVVSGSDFTEDAMAWYRQASG |
| (MSLN) | KERESVAFVSKDGKRILYLDSVRGRFTISRDIDKKTVYLQMD |
| (SEQ ID NO: 222) | NLKPEDTGVYYCNSAPGAARNYWGQGTQVTVSS |
| Exemplary anti-target | QVQPVESGGGLVQPGGSLRLSCVVSGSDFTEDAMAWYRQASG |
| (MSLN) | KERESVAFVSKDGKRILYLDSVRGRFTISRDIYKKTVYLQMD |
| (SEQ ID NO: 223) | NLKPEDTGVYYCNSAPGAARNVWGQGTQVTVSS |
| Exemplary anti-target | EVQLVESGGGLVQPGNSLRLSCAASGFTFSSFGMSWVRQAPG |
| (MSLN) | KGLEWVSSISGSGSDTLYADSVKGRFTISRDNAKTTLYLQMN |
| (SEQ ID NO: 224) | SLRPEDTAVYYCTIGGSLSRSSQGTLVTVSS |
| Exemplary anti-target | QVQIVESGGGLVQAGGSLRLSCVASGLTYSIVAVGWYRQAPG |
| (MSLN) | KEREMVADISPVGNTNYADSVKGRFTISKENAKNTVYLQMNS |
| (SEQ ID NO: 225) | LKPEDTAVYYCHIVRGWLDERPGPGPIVYWGQGTQVTVSS |
| Exemplary anti-target | QVQLVESGGGLVQTGGSLRLSCAASGLTFGVYGMEWFRQAPG |
| (MSLN) | KQREWVASHTSTGYVYYRDSVKGRFTISRDNAKSTVYLQMNS |
| (SEQ ID NO: 226) | LKPEDTAIYYCKANRGSYEYWGQGTQVTVSS |
| Exemplary anti-target | QVQLVESGGGLVQAGGSLRLSCAASTTSSINSMSWYRQAQGK |
| (MSLN) | QREPVAVITDRGSTSYADSVKGRFTISRDNAKNTVYLQMNSL |
| (SEQ ID NO: 227) | KPEDTAIYTCHVIADWRGYWGQGTQVTVSS |
| Exemplary anti-target | QVQLVESGGGLVQAGGSLRLSCAASGRTLSRYAMGWFRQAPG |
| (MSLN) | KERQFVAAISRSGGTTRYSDSVKGRFTISRDNAANTFYLQMN |
| (SEQ ID NO: 228) | NLRPDDTAVYYCNVRRRGWGRTLEYWGQGTQVTVSS |
| Exemplary anti-target | QVQLGESGGGLVQAGGSLRLSCAASGSIFSPNAMIWHRQAPG |
| (MSLN) | KQREPVASINSSGSTNYGDSVKGRFTVSRDIVKNTMYLQMNS |
| (SEQ ID NO: 229) | LKPEDTAVYYCSYSDFRRGTQYWGQGTQVTVSS |
| Exemplary anti-target | QVQLVESGGGLVPSGGSLRLSCAASGATSAITNLGWYRRAPG |
| (MSLN) | QVREMVARISVREDKEDYEDSVKGRFTISRDNTQNLVYLQMN |
| (SEQ ID NO: 230) | NLQPHDTAIYYCGAQRWGRGPGTTWGQGTQVTVSS |
| Exemplary anti-target | QVQLVESGGGLVQAGGSLRLSCAASGSTFRIRVMRWYRQAPG |
| (MSLN) | TERDLVAVISGSSTYYADSVKGRFTISRDNAKNTLYLQMNNL |
| (SEQ ID NO: 231) | KPEDTAVYYCNADDSGIARDYWGQGTQVTVSS |
| Exemplary anti-target | QVQLVESGGGLVQAGESRRLSCAVSGDTSKFKAVGWYRQAPG |
| (MSLN) | AQRELLAWINNSGVGNTAESVKGRFTISRDNAKNTVYLQMNR |
| (SEQ ID NO: 232) | LTPEDTDVYYCRFYRRFGINKNYWGQGTQVTVSS |
| Exemplary anti-target | QVQLVESGGGLVQAGGSLRLSCAASGSTFGNKPMGWYRQAPG |
| (MSLN) | KQRELVAVISSDGGSTRYAALVKGRFTISRDNAKNTVYLQME |
| (SEQ ID NO: 233) | SLVAEDTAVYYCNALRTYYLNDPVVFSWGQGTQVTVSS |
| Exemplary anti-target | QVQLVESGGGLVQAGGSLRLSCAASGSTSSINTMYWYRQAPG |
| (MSLN) | KERELVAFISSGGSTNVRDSVKGRFSVSRDSAKNIVYLQMNS |
| (SEQ ID NO: 234) | LTPEDTAVYYCNTYIPLRGTLHDYWGQGTQVTVSS |
| Exemplary anti-target | QVQLVESGGGLVQAGGSLRLSCVASGRTDRITTMGWYRQAPG |
| (MSLN) | KQRELVATISNRGTSNYANSVKGRFTISRDNAKNTVYLQMNS |
| (SEQ ID NO: 235) | LKPEDTAVYYCNARKWGRNYWGQGTQVTVSS |
| Exemplary anti-target | QVQLVESGGGLVQARGSLRLSCTASGRTIGINDMAWYRQAPG |
| (MSLN) | NQRELVATITKGGTTDYADSVDGRFTISRDNAKNTVYLQMNS |
| (SEQ ID NO: 236) | LKPEDTAVYYCNTKRREWAKDFEYWGQGTQVTVSS |
| Exemplary anti-target | QVQLVESGGGLVQAGGSLRLSCAASAIGSINSMSWYRQAPGK |
| (MSLN) | QREPVAVITDRGSTSYADSVKGRFTISRDNAKNTVYLQMNSL |
| (SEQ ID NO: 237) | KPEDTAIYTCHVIADWRGYWGQGTQVTVSS |
| Exemplary anti-target | QVQLVESGGGLVQAGGSLRLSCAASGSTSSINTMYWFRQAPG |
| (MSLN) | EERELVATINRGGSTNVRDSVKGRFSVSRDSAKNIVYLQMNR |
| (SEQ ID NO: 238) | LKPEDTAVYYCNTYIPYGGTLHDFWGQGTQVTVSS |
| Exemplary anti-target | QVQLVESGGGLVQAGGSLRLSCTTSTTFSINSMSWYRQAPGN |
| (MSLN) | QREPVAVITNRGTTSYADSVKGRFTISRDNARNTVYLQMDSL |
| (SEQ ID NO: 239) | KPEDTAIYTCHVIADWRGYWGQGTQVTVSS |
| Exemplary anti-target | QVQLVESGGGLVQAGGSLTLSCAASGSTFSIRAMRWYRQAPG |
| (MSLN) | TERDLVAVIYGSSTYYADAVKGRFTISRDNAKNTLYLQMNNL |
| (SEQ ID NO: 240) | KPEDTAVYYCNADTIGTARDYWGQGTQVTVSS |
| Exemplary anti-target | QVQLVESGGGLVQAGGSLRLSCVASGRTSTIDTMYWHRQAPG |
| (MSLN) | NERELVAYVTSRGTSNVADSVKGRFTISRDNAKNTAYLQMNS |
| (SEQ ID NO: 241) | LKPEDTAVYYCSVRTTSYPVDFWGQGTQVTVSS |
| Exemplary anti-target | QVQLVESGGGLVQAGGSLRLSCAASGSTSSINTMYWYRQAPG |
| (MSLN) | KERELVAFISSGGSTNVRDSVKGRESVSRDSAKNIVYLQMNS |
| (SEQ ID NO: 242) | LKPEDTAVYYCNTYIPYGGTLHDFWGQGTQVTVSS |
| Exemplary anti-target | QVQLVESGGGLVQPGGSLRLSCAASGGDWSANFMYWYRQAPG |
| (MSLN) | KQRELVARISGRGVVDYVESVKGRFTISRDNAKNTVYLQMNS |
| (SEQ ID NO: 243) | LKPEDTAVYYCAVASYWGQGTQVTVSS |
| Exemplary anti-target | EVQLVESGGGLVQPGGSLRLSCAASGGDWSANFMYWYRQAPG |
| (MSLN) | KQRELVARISGRGVVDYVESVKGRFTISRDNSKNTLYLQMNS |
| (SEQ ID NO: 244) | LRAEDTAVYYCAVASYWGQGTLVTVSS |
| Exemplary anti-target | EVQLVESGGGLVQPGGSLRLSCAASGGDWSANFMYWVRQAPG |
| (MSLN) | KGLEWVSRISGRGVVDYVESVKGRFTISRDNSKNTLYLQMNS |
| (SEQ ID NO: 245) | LRAEDTAVYYCAVASYWGQGTLVTVSS |
| Exemplary anti-target | EVQLVESGGGLVQAGGSLRLSCAASGSTSSINTMYWYRQAPG |
| (MSLN) | KERELVAFISSGGSTNVRDSVKGRFTISRDNSKNTLYLQMNS |
| (SEQ ID NO: 246) | LRAEDTAVYYCNTYIPYGGTLHDFWGQGTLVTVSS |
| Exemplary anti-target | EVQLVESGGGLVQPGGSLRLSCAASGSTSSINTMYWYRQAPG |
| (MSLN) | KERELVAFISSGGSTNVRDSVKGRFTISRDNSKNTLYLQMNS |
| (SEQ ID NO: 247) | LRAEDTAVYYCNTYIPYGGTLHDFWGQGTLVTVSS |
| Exemplary anti-target | EVQLVESGGGLVQPGGSLRLSCAASGSTSSINTMYWVRQAPG |
| (MSLN) | KGLEWVSFISSGGSTNVRDSVKGRFTISRDNSKNTLYLQMNS |
| (SEQ ID NO: 248) | LRAEDTAVYYCNTYIPYGGTLHDFWGQGTLVTVSS |
| Exemplary anti-target | QVQLVESGGGVVQAGGSLRLSCAASGSTFSIRAMRWYRQAPG |
| (MSLN) | TERDLVAVIYGSSTYYADAVKGRFTISRDNSKNTLYLQMNSL |
| (SEQ ID NO: 249) | RAEDTAVYYCNADTIGTARDYWGQGTLVTVSS |
| Exemplary anti-target | QVQLVESGGGVVQPGGSLRLSCAASGSTFSIRAMRWYRQAPG |
| (MSLN) | KERELVAVIYGSSTYYADAVKGRFTISRDNSKNTLYLQMNSL |
| (SEQ ID NO: 250) | RAEDTAVYYCNADTIGTARDYWGQGTLVTVSSGG |
| Exemplary anti-target | QVQLVESGGGVVQPGGSLRLSCAASGSTFSIRAMRWVRQAPG |
| (MSLN) | KGLEWVSVIYGSSTYYADAVKGRFTISRDNSKNTLYLQMNSL |
| (SEQ ID NO: 251) | RAEDTAVYYCNADTIGTARDYWGQGTLVTVSSGG |
| Exemplary anti-target | EVQLVESGGGLVQAGGSLRLSCVASGRTSTIDTMYWHRQAPG |
| (MSLN) | NERELVAYVTSRGTSNVADSVKGRFTISRDNSKNTLYLQMNS |
| (SEQ ID NO: 252) | LRAEDTAVYYCSVRTTSYPVDFWGQGTLVTVSGG |
| Exemplary anti-target | EVQLVESGGGLVQPGGSLRLSCAASGRTSTIDTMYWHRQAPG |
| (MSLN) | KERELVAYVTSRGTSNVADSVKGRFTISRDNSKNTLYLQMNS |
| (SEQ ID NO: 253) | LRAEDTAVYYCSVRTTSYPVDFWGQGTLVTVSS |
| Exemplary anti-target | EVQLVESGGGLVQPGGSLRLSCAASGRTSTIDTMYWVRQAPG |
| (MSLN) | KGLEWVSYVTSRGTSNVADSVKGRFTISRDNSKNTLYLQMNS |
| (SEQ ID NO: 254) | LRAEDTAVYYCSVRTTSYPVDFWGQGTLVTVSS |
| Exemplary anti-target | QVQLVESGGGVVQAGGSLTLSCAASGSTFSIRAMRWYRQAPG |
| (MSLN) | TERDLVAVIYGSSTYYADAVKGRFTISRDNSKNTLYLQMNSL |
| (SEQ ID NO: 255) | RAEDTAVYYCNADTIGTARDYWGQGTLVTVSS |
| Exemplary anti-target | QVQLVESGGGVVQAGGSLRLSCAASGSTFSIRAMRWYRQAPG |
| (MSLN) | TERDLVAVIYGSSTYYADAVKGRFTISRDNSKNTLYLQMNSL |
| (SEQ ID NO: 256) | RAEDTAVYYCNADTIGTARDYWGQGTLVTVSS |
| Exemplary anti-target | QVQLVESGGGVVQPGGSLRLSCAASGSTESIRAMRWYRQAPG |
| (MSLN) | KERELVAVIYGSSTYYADAVKGRFTISRDNSKNTLYLQMNSL |
| (SEQ ID NO: 257) | RAEDTAVYYCNADTIGTARDYWGQGTLVTVSS |
| Exemplary anti-target | QVQLVESGGGVVQPGGSLRLSCAASGSTFSIRAMRWVRQAPG |
| (MSLN) | KGLEWVSVIYGSSTYYADAVKGRFTISRDNSKNTLYLQMNSL |
| (SEQ ID NO: 258) | RAEDTAVYYCNADTIGTARDYWGQGTLVTVSS |
| Exemplary anti-target | QVQLQESGGGLVQAGGSLRLSCAASGSIFSIASMGWYRQAPG |
| (DLL3) | KQRELVAVITSFSSTNYADSVKGRFTISRDNAKNTVYLQMNS |
| (SEQ ID NO: 306) | LKPEDTGVYYCNARYFERTDWGQGTQVTVSS |
| Exemplary anti-target | QVQLQESGGGLVQAGGSLRLSCAAPGSIFSIASMGWYRQAPG |
| (DLL3) | KQRELVAVITSFSSTNYADSVKGRFTISRDNAKNTVYLQMNS |
| (SEQ ID NO: 307) | LKPEDTGVYYCNARYFERTDWGQGTQVTVSS |
| Exemplary anti-target | QVQLQESGGGLVQAGGSLRLSCAASGSIFSIASMAWYRQAPG |
| (DLL3) | KQRELVAAITSFSSTNYADSVKGRFTISRDNAKNTVYLQMNS |
| (SEQ ID NO: 308) | LKPEDTGVYYCNARYFERTDWGQGTQVTVSS |
| Exemplary anti-target | QVQLQESGGGLVQAGGSLRLSCAASESIFSINVMAWHRQAPG |
| (DLL3) | KQRELVARITSGGSTNYADSVKGRFTISRDNAKNTVYLQMNS |
| (SEQ ID NO: 309) | LKPEDTGVYYCGAYQGLYAYWGQGTQVTVSS |
| Exemplary anti-target | QVQLQESGGGLVQAGGSLRLSCVASGSSFSITSMAWYRQAPG |
| (DLL3) | KQRDLVAAITSFGSTNYADSVKDRFTISRDNAKNTVYLQMNS |
| (SEQ ID NO: 310) | LKPEDTAVYYCNGRVFDHVYWGQGTQVTVSS |
| Exemplary anti-target | QVQLQESGGGLVQAGGSLKLSCAASSSIFSISSMSWYRQAPG |
| (DLL3) | KQRELVAAITTFDYTNYADSVKGRFTISRDNAKNMMYLQMNS |
| (SEQ ID NO: 311) | LKPEDTAVYLCNARAFGRDYWGQGTQVTVSS |
| Exemplary anti-target | QVQLQESGGGLVQAGGSLKLSCAASSSIFSISSMSWYRQAPG |
| (DLL3) | KQRELVAAITSFGSTNYADSVKGRFTISRDNAKNMMYLQMNS |
| (SEQ ID NO: 312) | LKPEDTAVYRCNARTMGRDYWGQGTQVTVSS |
| Exemplary anti-target | QVQLQESGGGLVQPGGSLRLSCAASGSTLNIKIMAWHRQAPG |
| (DLL3) | KQRELVATLTSGGNTNYADSVKGRFTISRDNAKNTVYLQMNS |
| (SEQ ID NO: 313) | LQPEDTAVYYCGLWDGVGGAYWGRGTQVTVSS |
| Exemplary anti-target | QVQLQESGGGLVQPGGSLRISCAASGSTLNIKIMAWHRQAPG |
| (DLL3) | KQRELVATLTSGGNTNYADSVKGRFTISRDNAKNTVYLQMNS |
| (SEQ ID NO: 314) | LQPEDTAVYYCGLWDGVGGAYWGRGTQVTVSS |
| Exemplary anti-target | QVQLQESGGGLVQAGGSLRLSCAASGSTFNIKTMAWHRQAPG |
| (DLL3) | NQRELVATLTSGGNTNYADSVKGRFTISRDNAKNTVYLQMNS |
| (SEQ ID NO: 315) | LKPEDTAVYYCGLWNGVGGAYWGRGTQVTVSS |
| Exemplary anti-target | QVQLQDGGGLVQPGGSLRLSCAASGSTFNIKLMAWHRQAPGN |
| (DLL3) | QRELVATLTSGGNTNYADSVKGRFTISRDNASNIVYLQMNSL |
| (SEQ ID NO: 316) | KPEDTAVYYCGLWDGVGGAYWGRGTQVTVSS |
| Exemplary anti-target | QVQLQESGGGLVQAGGSLRLSCAASGSTENFKIMAWHRQAPG |
| (DLL3) | KQRELVASLTSEGLTNYRDSVKGRFTISRDNAKNTVYLQMNN |
| (SEQ ID NO: 317) | LKPEDTAVYYCGLWDGVGGAYWGRGTQVTVSS |
| Exemplary anti-target | QVQLQESGGGLVQPGGSLRLSCAASGFMFSSYSMSWYRQAPG |
| (DLL3) | KQRELVAAITTWGSTNYADSVKGRFTISRDNAKNTVWLQMNS |
| (SEQ ID NO: 318) | LEPEDTAVYFCNARSWNNYWGQGTQVTVSS |
| Exemplary anti-target | QVQLQESGGGLVQVGGSLRLSCAASGFMESSYSMSWYRQAPG |
| (DLL3) | KQRELVAAITSYGSTNYADSVKGRFTISRDNAKNTVWLQMNS |
| (SEQ ID NO: 319) | LKPEDTAVYFCNARSWNNYWGQGTQVTVSS |
| Exemplary anti-target | QVQLQESGGGLVQPGGSLRLSCAASGFTFSSHSMSWYRQAPG |
| (DLL3) | KQRELVAAITTYGSTNYIDSVKGRFTISRDNTKNTVYLQMNS |
| (SEQ ID NO: 320) | LKPEDTAVYFCNARSWNNYWGQGTQVTVSS |
| Exemplary anti-target | QVQLQESGGGLVQAGGSLRLSCVASGSSFSHNTMGWYRQAPG |
| (DLL3) | KQRDLVARITTFGTTNYADSVKGRFTISRDNAKNTVYLQMNS |
| (SEQ ID NO: 321) | LKPEDTAVYYCNGESFGRIWYNWGQGTQVTVSS |
| Exemplary anti-target | QVQLQESGGGLVQAGASLRLTCTASGGRFSYATMGWSRQAPG |
| (DLL3) | KQREMVARITSSGFSTNYADSVKGRFTISRDNAKNAVYLQMD |
| (SEQ ID NO: 322) | SLKPEDTAVYYCNAQHFGTDSWGQGTQVTVSS |
| Exemplary anti-target | QVQLQESGGGLVQAGASLRLTCTASGSRFSYATMGWSRQAPG |
| (DLL3) | KQRELVARITSSGESTNYADSVKGRFTISRDNAKNAVYLQMD |
| (SEQ ID NO: 323) | SLKPEDTAVYYCNAQQFGTDSWGQGTQVTVSS |
| Exemplary anti-target | QVQLQESGGGLVQAGGSLRLSCAASGSTFTSNVMGWHRQAPG |
| (DLL3) | KQRELVANMHSGGSTNYADSVKGRFTISRDNAKNIVYLQMNN |
| (SEQ ID NO: 324) | LKIEDTAVYYCRWYGIQRAEGYWGQGTQVTVSS |
| Exemplary anti-target | QVQLQESGGGLVVAGGSLRLSCAASGSSVSFLSMAWYRQAPG |
| (DLL3) | KKRELVAGISVDGSTNYADSVKGRFTVSRDNAKNTVYLQMNS |
| (SEQ ID NO: 325) | LQPEDTAVYYCYAYRWVGRDTYWGQGTQVTVSS |
| Exemplary anti-target | QVQLQESGGGLVVAGGSLRLSCAASGSSVSFLSMAWYRQAPG |
| (DLL3) | KKRELVAGISVDGSTNYADSVKGRFTISRDNAKNTVYLQMNS |
| (SEQ ID NO: 326) | LQPEDTAVYYCYAYRWVGRDTYWGQGTQVTVSS |
| Exemplary anti-target | QVQLQESGGGLVVSGGSLRLSCAASGSSVSFLSMAWYRQAPG |
| (DLL3) | KKRELVAGISVDGSTNYADSVKGRFTISRDNAKNTVYLQMNS |
| (SEQ ID NO: 327) | LQPEDTAAYYCYAYRWVGRDTYWGQGTQVTVSS |
| Exemplary anti-target | QVQLQESGGGLVVAGGSLRLSCAASGSSVSFLSMAWYRQAPG |
| (DLL3) | KKRELVAGISVDGSTNYADSVKGRFTISRDNAENTVYLQMNS |
| (SEQ ID NO: 328) | LQPEDTAVYYCYAYRWEGRDTYWGQGTQVTVSS |
| Exemplary anti-target | QVQLQESGGGLVVAGGSLRLSCAASGSSVSFLSMAWYRQAPG |
| (DLL3) | KKRELVAGISVDGSTNYADSVKGRFTISRDNAENTVYLQMNS |
| (SEQ ID NO: 329) | LQPEDTAVYYCYAYRWEGRNTYWGQGTQVTVSS |
| Exemplary anti-target | QVQLQESGGGLVQPGGSLRLSCAASGSSVSFLSMAWYRQAPG |
| (DLL3) | KKRELVAGISTDGSTNYVDSVKGRFTISRDNAKNTVYLQMNS |
| (SEQ ID NO: 330) | LQPEDTAVYYCYAYRWVGRYTYWGQGTQVTVSS |
| Exemplary anti-target | QVQLQESGGGLVVAGGSLRLSCAASGSSVSFLSMAWYRQAPG |
| (DLL3) | KKRELVAGISTDGTTNYVDSVKDRFTISRDNAKNTVYLQMNS |
| (SEQ ID NO: 331) | LQPEDTAAYYCYAYRWVGRDTYWGQGTQVTVSS |
| Exemplary anti-target | QVQLQESGGGLVVAGGSLRLSCAASGSSVSFLSIAWYRQAPG |
| (DLL3) | KKRELVAGISTDGTTNYVDSVKDRFTISRDNAKNTVYLQMNS |
| (SEQ ID NO: 332) | LQPEDTAAYYCYAYRWVGRDTYWGQGTQVTVSS |
| Exemplary anti-target | QVQLQESGGGLVQAGGSLTLSCAASGSSVSFLSMAWYRQAPG |
| (DLL3) | KKRELVAGISTDGTTNYVDSVKDRFTISRDNAKNTVYLQMNS |
| (SEQ ID NO: 333) | LQPEDTAAYYCYAYRWVGRDTYWGQGTQVTVSS |
| Exemplary anti-target | QVQLQESGGGLVQAGGSLRLSCAASGSSVSFLSMAWYRQAPG |
| (DLL3) | KKRELVAGISTDGSTNYADSVKGRFTISEGNAKNTVDLQMNS |
| (SEQ ID NO: 334) | LQPEDTAVYYCYAYRWVDRYTYWGQGTQVTVSS |
| Exemplary anti-target | QVQLQESGGGLVVAGGSLRLSCAASGSSVSFLSMAWYRQAPG |
| (DLL3) | KKRELVAGISTDGSTNYADSVKGRFTISEDNAKNTVDLQMNS |
| (SEQ ID NO: 335) | LQPEDTAVYYCYAYRWIDRYTYWGQGTQVTVSS |
| Exemplary anti-target | QVQLQESGGGLVQPGGSLRLSCAASGSSVSFLSMAWYRQAPG |
| (DLL3) | KKRELVAGISTDGSTNYADSVKGRFTISEDNAKNTVDLQMNS |
| (SEQ ID NO: 336) | LQPEDTAVYYCYAYRWVDRYTYWGQGTQVTVSS |
| Exemplary anti-target | QVQLQESGGGLVVAGGSLRLSCAASGSSVSFLSMAWYRQAPG |
| (DLL3) | KKRELVAGISSDGSTNYVDSVKGRFTISRDNAKNIVFLQMNS |
| (SEQ ID NO: 337) | LQPQDTAVYYCYAYRWVGRDTYWGQGTQVTVSS |
| Exemplary anti-target | QVQLQESGGGLVVAGGSLRLSCAASGSSVSFLSMAWYRQAPG |
| (DLL3) | KKRELVAGISADGSTDYIDSVKGRFTISRDSANNTMYLQMNS |
| (SEQ ID NO: 338) | LQPEDTAVYYCYAYRWTTRYTYWGQGTQVTVSS |
| Exemplary anti-target | QVQLQESGGGLVQPGGSLRLSCAASGFTLDYYAIGWYRQAPG |
| (DLL3) | KKRELVAGISSDGSTHYVDSVKGRFAISRDNAENTVYLQMND |
| (SEQ ID NO: 339) | LQPDDTAVYYCYAYRWVGGYTYWGQGTQVTVSS |
| Exemplary anti-target | QVQLQESGGGLVQAGGSLRLSCVASGSTSSINAMGWYRRAPG |
| (DLL3) | KQRELVAGISSDGSKNYADSVKGRFTISRDNAKNTVYLQMNS |
| (SEQ ID NO: 340) | LKPEDTAVYYCYYFRTVAASSMQYWGQGTQVTVSS |
| Exemplary anti-target | QVQLQESGGGLVQAGGSLRLSCVASGSTSSINAMGWYRRAPG |
| (DLL3) | KQRELVAGISSDGSKVYADSVKGRFTISRDNAKNTVYLQMNS |
| (SEQ ID NO: 341) | LKPEDTAVFYCYYFRTVSGSSMRYWGQGTQVTVSS |
| Exemplary anti-target | QVQLQESGGGLVQAGGSLRLSCAASGITSSVYSMGWYRQAPG |
| (DLL3) | KQRELVAGSSSDGSTHYVDSVRGRFTISRDNAKNTVYLQMSS |
| (SEQ ID NO: 342) | LKPEDTAVYYCYANRGFAGAPSYWGQGTQVTVSS |
| Exemplary anti-target | QVQLQESGGGLVQAGGSLRLSCAASGRTSMENSMGWHRQAPG |
| (DLL3) | KQRELVAIIRSGGSSNYADTVKGRFTISRDNTKNTVYLQMND |
| (SEQ ID NO: 343) | LKPEDTAVYYCFYYFQSSYWGQGTQVTVSS |
| Exemplary anti-target | QVQLQESGGGLVQAGGSLRLSCAASGRTSMVNSMGWHRQAPG |
| (DLL3) | KQRELVALITSGGSSNYADTVKGRFTISRDNTKNTVYLQMND |
| (SEQ ID NO: 344) | LKPEDTAVYYCFYYFQSSYWGQGTQVTVSS |
| Exemplary anti-target | QVQLQESGGGLVQAGGSLRLSCAASGSVSMENSMGWHRQPPG |
| (DLL3) | KQRELVAIITSGGSSNYADTVKGRFTISRDNTKNTVYLQMND |
| (SEQ ID NO: 344) | LKPEDTAVYYCFYYFQSSYWGQGTQVTVSS |
| Exemplary anti-target | QVQLQESGGGLVQAGGSLRLSCTASGSIFSIAVMGWYRQVPG |
| (DLL3) | KRREWVATIFDGSYTNYADSVKGRFTISRDNARNKVYLQMNN |
| (SEQ ID NO: 345) | LKPEDTAVYYCQTHWTQGSVPKESWGQGTQVTVSS |
| Exemplary anti-target | QVQLQESGGGLVQAGGSLRLSCVASSGIFSDMSMVWYRQAPG |
| (DLL3) | KQRELVASITTFGSTNYADPVKGRFTISRDNAKNTVYLQMNS |
| (SEQ ID NO: 346) | LKPEDTAVYYCSGRSYSSDYWGRGTQVTVSS |
| Exemplary anti-target | QVQLQESGGGLVQAGGSLRLSCVASGSISSIIVMGWSRQAPG |
| (DLL3) | KQRESVATITRDGTRNYADSLKGRFTISRDNAKNTSYLQINS |
| (SEQ ID NO: 347) | LKPEDTAVYSCYARYGDINYWGKGTQVTVSS |
| Exemplary anti-target | QVQLQESGGGLVQAGGSLRLSCVASGSISSIIVMGWSRQAPG |
| (DLL3) | KQRESLATISRGGTRTYADSVKGRFTISRDNAKNTSYLQMNS |
| (SEQ ID NO: 348) | LKPEDTAVYSCYARYGDINYWGKGTQVTVSS |
| Exemplary anti-target | QVQLQESGGGLVQAGGSLRLSCVASGSIFTTNSMGWHRQGPG |
| (DLL3) | KQRELVALIGSAGSTKYADSVKGRFTISRDNAKNTVSLQMDS |
| (SEQ ID NO: 349) | LKPEDTAVYYCFYYDSRSYWGQGTQVTVSS |
| Exemplary anti-target | QVQLQESGGGMVQPGGSLRLSCAASGSREISTMGWHRQAPGK |
| (DLL3) | QRELAARITSGGITKYADSVKGRFTISRDNAKKTVYLQMNSL |
| (SEQ ID NO: 350) | KSEDTAVYYCFAYDNINAYWGQGTQVTVSS |
| Exemplary anti-target | QVQLQESGGGWVQAGGSLRLSCAASGSREISTMGWHRQAPGK |
| (DLL3) | QRELAARITSGGITKYADSVKGRFTISRDNAKKTVYLQMNSL |
| (SEQ ID NO: 351) | KSEDTAVYYCFAYDNINAYWGQGTQVTVSS |
| Exemplary anti-target | QVQLQESGGGWVQAGGSLRLSCTASGSREISTMGWHRQAPGK |
| (DLL3) | QRELAARITSGGITKYADSVKGRFTISRDNAKKTVYLQMDSL |
| (SEQ ID NO: 352) | KSEDTAVYYCFAYDNINAYWGQGTQVTVSS |
| Exemplary anti-target | QVQLQESGGGSVQAGRSLGLSCAASGSREISTMGWHRQAPGK |
| (DLL3) | QRELAARITSGGITKYADSVKGRFTISRDNAKKTVYLQMNSL |
| (SEQ ID NO: 353) | KSEDTAVYYCFAYDNINAYWGQGTQVTVSS |
| Exemplary anti-target | QVQLQESGGGLVQAGGSLRLSCTASGSIFRGAAMYWHRQAPG |
| (DLL3) | KQRELVAAITTSGNTSYADSVKGRFTISRDNAKNTMYLQIIS |
| (SEQ ID NO: 354) | LKPEDTAVYYCAFWIAGKAYWGQGTQVTVSS |
| Exemplary anti-target | QVQLQESGGGLVQPGGSLRLSCAASGSISSENFMSWHRQAPG |
| (DLL3) | KERELAGVITRGGATNYADSVKGRFTISRDNVKNTVYLQMNG |
| (SEQ ID NO: 355) | LKPEDTAVYYCHGRSQLGSTWGQGTQVTVSS |
| Exemplary anti-target | QVQLQESGGGLVQAGGSLRLSCLASGTIFTASTMGWHRQPPG |
| (DLL3) | KQRELVASIAGDGRTNYAESTEGRFTISRDDAKNTMYLQMNS |
| (SEQ ID NO: 356) | LKPEDTAVYYCYAYYLDTYAYWGQGTQVTVSS |
| Exemplary anti-target | EVQLVESGGGLVQPGGSLTLSCAASGSIFSIASMGWYRQAPG |
| (DLL3) | KQRELVAVITSFSSTNYADSVKGRFTISRDNAKNSVYLQMNS |
| (SEQ ID NO: 357) | LRAEDTAVYYCNARYFERTDWGQGTLVTVSS |
| Exemplary anti-target | EVQLVESGGGLVQPGGSLTLSCAASGRTSMFNSMGWHRQAPG |
| (DLL3) | KQRELVAIIRSGGSSNYADTVKGRFTISRDNAKNSVYLQMNS |
| (SEQ ID NO: 358) | LRAEDTAVYYCFYYFQSSYWGQGTLVTVSS |
| Exemplary anti-target | EVQLVESGGGLVQPGGSLTLSCAASGSSVSFLSMAWYRQAPG |
| (DLL3) | KKRELVAGISVDGSTNYADSVKGRFTISRDNAKNSVYLQMNS |
| (SEQ ID NO: 359) | LRAEDTAVYYCYAYRWVGRDTYWGQGTLVTVSS |
| Exemplary anti-target | EVQLVESGGGLVQPGGSLTLSCTASGSIFRGAAMYWHRQAPG |
| (DLL3) | KQRELVAAITTSGNTSYADSVKGRFTISRDNAKNSMYLQMNS |
| (SEQ ID NO: 360) | LRAEDTAVYYCAFWIAGKAYWGQGTLVTVSS |
| Exemplary anti-target | EVQLVESGGGLVQPGGSLTLSCAASGSTFNIKTMAWHRQAPG |
| (DLL3) | NQRELVATLTSGGNTNYADSVKGRFTISRDNAKNSVYLQMNS |
| (SEQ ID NO: 361) | LRAEDTAVYYCGLWNGVGGAYWGQGTLVTVSS |
| Exemplary anti-target | EVQLVESGGGLVQPGGSLTLSCAASGSTLNIKIMAWHRQAPG |
| (DLL3) | KQRELVATLTSGGNTNYADSVKGRFTISRDNAKNSVYLQMNS |
| (SEQ ID NO: 362) | LRAEDTAVYYCGLWDGVGGAYWGQGTLVTVSS |
| Exemplary anti-target | EVQLVESGGGLVQPGGSLTLSCAASSSIFSISSMSWYRQAPG |
| (DLL3) | KQRELVAAITTFDYTNYADSVKGRFTISRDNAKNSMYLQMNS |
| (SEQ ID NO: 363) | LRAEDTAVYYCNARAFGRDYWGQGTLVTVSS |
| Exemplary anti-target | EVQLVESGGGLVQPGGSLTLSCVASGSTSSINAMGWYRRAPG |
| (DLL3) | KQRELVAGISSDGSKNYADSVKGRFTISRDNAKNSVYLQMNS |
| (SEQ ID NO: 364) | LRAEDTAVYYCYYFRTVAASSMQYWGQGTLVTVSS |
| Exemplary anti-target | EVQLVESGGGLVQPGGSLTLSCAASGFMESSYSMSWYRQAPG |
| (DLL3) | KQRELVAAITSYGSTNYADSVKGRFTISRDNAKNSVYLQMNS |
| (SEQ ID NO: 365) | LRAEDTAVYYCNARSWNNYWGQGTLVTVSS |
| Exemplary anti-target | EVQLVESGGGLVQPGGSLTLSCAASGSVSMENSMGWHRQPPG |
| (DLL3) | KQRELVAIITSGGSSNYADTVKGRFTISRDNAKNSVYLQMNS |
| (SEQ ID NO: 366) | LRAEDTAVYYCFYYFQSSYWGQGTLVTVSS |
| Exemplary anti-target | EVQLVESGGGLVQPGGSLTLSCTASGGRFSYATMGWSRQAPG |
| (DLL3) | KQREMVARITSSGFSTNYADSVKGRFTISRDNAKNSVYLQMN |
| (SEQ ID NO: 367) | SLRAEDTAVYYCNAQHFGTDSWGQGTLVTVSS |
| Exemplary anti-target | EVQLVESGGGLVQPGGSLTLSCAASGSISSENFMSWHRQAPG |
| (DLL3) | KERELAGVITRGGATNYADSVKGRFTISRDNAKNSVYLQMNS |
| (SEQ ID NO: 368) | LRAEDTAVYYCHGRSQLGSTWGQGTLVTVSS |
| Exemplary anti-target | EVQLVESGGGLVQPGGSLTLSCAASESIFSINVMAWHRQAPG |
| (DLL3) | KQRELVARITSGGSTNYADSVKGRFTISRDNAKNSVYLQMNS |
| (SEQ ID NO: 369) | LRAEDTAVYYCGAYQGLYAYWGQGTLVTVSS |
| Exemplary anti-target | EVQLVESGGGLVQPGGSLTLSCAASGSREISTMGWHRQAPGK |
| (DLL3) | QRELAARITSGGITKYADSVKGRFTISRDNAKNSVYLQMNSL |
| (SEQ ID NO: 370) | RAEDTAVYYCFAYDNINAYWGQGTLVTVSS |
| Exemplary anti-target | EVQLVESGGGLVQPGGSLTLSCTASGSIFSIAVMGWYRQVPG |
| (DLL3) | KRREWVATIFDGSYTNYADSVKGRFTISRDNAKNSVYLQMNS |
| (SEQ ID NO: 371) | LRAEDTAVYYCQTHWTQGSVPKESWGQGTLVTVSS |
| Exemplary anti-target | EVQLVESGGGLVQPGGSLTLSCVASGSTSSINAMGWYRRAPG |
| (DLL3) | KQRELVAGISSDGSKVYADSVKGRFTISRDNAKNSVYLQMNS |
| (SEQ ID NO: 372) | LRAEDTAVYYCYYFRTVSGSSMRYWGQGTLVTVSS |
| Exemplary anti-target | EVQLVESGGGLVQPGGSLTLSCVASSGIFSDMSMVWYRQAPG |
| (DLL3) | KQRELVASITTFGSTNYADPVKGRFTISRDNAKNSVYLQMNS |
| (SEQ ID NO: 373) | LRAEDTAVYYCSGRSYSSDYWGQGTLVTVSS |
| Exemplary anti-target | EVQLVESGGGLVQPGGSLTLSCAASGFMESSYSMSWYRQAPG |
| (DLL3) | KQRELVAAITTWGSTNYADSVKGRFTISRDNAKNSVYLQMNS |
| (SEQ ID NO: 374) | LRAEDTAVYYCNARSWNNYWGQGTLVTVSS |
| Exemplary anti-target | EVQLVESGGGLVQPGGSLTLSCTASGSRFSYATMGWSRQAPG |
| (DLL3) | KQRELVARITSSGESTNYADSVKGRFTISRDNAKNSVYLQMN |
| (SEQ ID NO: 375) | SLRAEDTAVYYCNAQQFGTDSWGQGTLVTVSS |
| Exemplary anti-target | EVQLVESGGGLVQPGGSLTLSCAASGSTFTSNVMGWHRQAPG |
| (DLL3) | KQRELVANMHSGGSTNYADSVKGRFTISRDNAKNSVYLQMNS |
| (SEQ ID NO: 376) | LRAEDTAVYYCRWYGIQRAEGYWGQGTLVTVSS |
| Exemplary anti-target | EVQLVESGGGLVQPGGSLTLSCAASGSSVSFLSMAWYRQAPG |
| (DLL3) | KKRELVAGISTDGSTNYVDSVKGRFTISRDNAKNSVYLQMNS |
| (SEQ ID NO: 377) | LRAEDTAVYYCYAYRWVGRYTYWGQGTLVTVSS |
| Exemplary anti-target | EVQLVESGGGLVQPGGSLTLSCVASGSISSIIVMGWSRQAPG |
| (DLL3) | KQRESVATITRDGTRNYADSLKGRFTISRDNAKNSSYLQMNS |
| (SEQ ID NO: 378) | LRAEDTAVYYCYARYGDINYWGQGTLVTVSS |
| Exemplary anti-target | EVQLVESGGGLVQPGGSLTLSCAASGSSVSFLSMAWYRQAPG |
| (DLL3) | KKRELVAGISADGSTDYIDSVKGRFTISRDNAKNSVYLQMNS |
| (SEQ ID NO: 379) | LRAEDTAVYYCYAYRWTTRYTYWGQGTLVTVSS |
| Exemplary anti-target | EVQLVESGGGLVQPGGSLTLSCLASGTIFTASTMGWHRQPPG |
| (DLL3) | KQRELVASIAGDGRTNYAESTEGRFTISRDNAKNSMYLQMNS |
| (SEQ ID NO: 380) | LRAEDTAVYYCYAYYLDTYAYWGQGTLVTVSS |
| Exemplary anti-target | EVQLVESGGGLVQPGGSLTLSCAASGSSVSFLSMAWYRQAPG |
| (DLL3) | KKRELVAGISVDGSTNYADSVKGRFTISRDNAKNSVYLQMNS |
| (SEQ ID NO: 381) | LRAEDTAVYYCYAYRWEGRNTYWGQGTLVTVSS |
| Exemplary anti-target | EVQLVESGGGLVQPGGSLTLSCVASGSSFSHNTMGWYRQAPG |
| (DLL3) | KQRDLVARITTFGTTNYADSVKGRFTISRDNAKNSVYLQMNS |
| (SEQ ID NO: 382) | LRAEDTAVYYCNGESFGRIWYNWGQGTLVTVSS |
| Exemplary anti-target | EVQLVESGGGLVQPGGSLTLSCVASGSISSIIVMGWSRQAPG |
| (DLL3) | KQRESLATISRGGTRTYADSVKGRFTISRDNAKNSSYLQMNS |
| (SEQ ID NO: 383) | LRAEDTAVYYCYARYGDINYWGQGTLVTVSS |
| Exemplary anti-target | EVQLVESGGGLVQPGGSLTLSCVASGSSFSITSMAWYRQAPG |
| (DLL3) | KQRDLVAAITSFGSTNYADSVKDRFTISRDNAKNSVYLQMNS |
| (SEQ ID NO: 384) | LRAEDTAVYYCNGRVFDHVYWGQGTLVTVSS |
| Exemplary anti-target | EVQLVESGGGLVQPGGSLTLSCAASGRTSMVNSMGWHRQAPG |
| (DLL3) | KQRELVALITSGGSSNYADTVKGRFTISRDNAKNSVYLQMNS |
| (SEQ ID NO: 385) | LRAEDTAVYYCFYYFQSSYWGQGTLVTVSS |
| Exemplary anti-target | EVQLVESGGGLVQPGGSLTLSCAASGSTENFKIMAWHRQAPG |
| (DLL3) | KQRELVASLTSEGLTNYRDSVKGRFTISRDNAKNSVYLQMNS |
| (SEQ ID NO: 386) | LRAEDTAVYYCGLWDGVGGAYWGQGTLVTVSS |
| Exemplary anti-target | EVQLVESGGGLVQPGGSLTLSCAASGFTLDYYAIGWYRQAPG |
| (DLL3) | KKRELVAGISSDGSTHYVDSVKGRFTISRDNAKNSVYLQMNS |
| (SEQ ID NO: 387) | LRAEDTAVYYCYAYRWVGGYTYWGQGTLVTVSS |
| Exemplary anti-target | EVQLVESGGGLVQPGGSLTLSCVASGSIFTTNSMGWHRQGPG |
| (DLL3) | KQRELVALIGSAGSTKYADSVKGRFTISRDNAKNSVYLQMNS |
| (SEQ ID NO: 388) | LRAEDTAVYYCFYYDSRSYWGQGTLVTVSS |
| Exemplary anti-target | EVQLVESGGGLVQPGGSLTLSCAASGITSSVYSMGWYRQAPG |
| (DLL3) | KQRELVAGSSSDGSTHYVDSVRGRFTISRDNAKNSVYLQMNS |
| (SEQ ID NO: 389) | LRAEDTAVYYCYANRGFAGAPSYWGQGTLVTVSS |
| Exemplary anti-target | EVQLVESGGGLVQPGGSLTLSCAASSSIFSISSMSWYRQAPG |
| (DLL3) | KQRELVAAITSFGSTNYADSVKGRFTISRDNAKNSMYLQMNS |
| (SEQ ID NO: 390) | LRAEDTAVYYCNARTMGRDYWGQGTLVTVSS |
| Exemplary anti-target | EVQLVESGGGLVQPGGSLTLSCVASGFTSSINAMGWYRRAPG |
| (DLL3) | KQRELVAGISSDGSFVYADSVKGRFTISRDNAKNSVYLQMNS |
| (SEQ ID NO: 391) | LRAEDTAVYYCYYFRHVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 392) | LRAEDTAVYYCYYFRTVSGSSSRYWGQGTLVTVSS |
| Exemplary anti-target | EVQLVESGGGLVQPGGSLTLSCVASGSTSSINAMGWYRRAPG |
| (DLL3) | KQRELVAGISSDGSKVYEDSVKGRFTISRDNAKNSVYLQMNS |
| (SEQ ID NO: 393) | LRAEDTAVYYCYYFRTVSGSSMRYWGQGTLVTVSS |
| Exemplary anti-target | EVQLVESGGGLVQPGGSLTLSCVASGSPSSINAMGWYRRAPG |
| (DLL3) | KQRELSAGISSDGSKVYADSVKGRFTISRDNAKNSVYLQMNS |
| (SEQ ID NO: 394) | LRAEDTAVYYCYYFRTVRGSSMSYWGQGTLVTVSS |
| Exemplary anti-target | EVQLVESGGGLVQPGGSLTLSCVASGSTSSINAMGWYRRAPG |
| (DLL3) | KQRELAAGISSDGSSVYADSVKGRFTISRDNAKNSVYLQMNS |
| (SEQ ID NO: 395) | LRAEDTAVYYCYYFRTVSGSSKRYWGQGTLVTVSS |
| Exemplary anti-target | EVQLVESGGGLVQPGGSLTLSCVASGSISSINAMGWYRRAPG |
| (DLL3) | KQRELVAGISSDGSKVYADSVKGRFTISRDNAKNSVYLQMNS |
| (SEQ ID NO: 396) | LRAEDTAVYYCYYFRMVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKLYADSVKGRFTISRDNAKNSVYLQMNS |
| (SEQ ID NO: 397) | LRAEDTAVYYCYYFRTVQGSSMRYWGQGTLVTVSS |
| Exemplary anti-target | EVQLVESGGGLVQPGGSLTLSCVASGSTSSINAYGWYRRAPG |
| (DLL3) | KQRELVAGISSDGSKVYADSVKGRFTISRDNAKNSVYLQMNS |
| (SEQ ID NO: 398) | LRAEDTAVYYCYYFRTVYGSSMRYWGQGTLVTVSS |
| Exemplary anti-target | EVQLVESGGGLVQPGGSLTLSCVASGSTSSINAIGWYRRAPG |
| (DLL3) | KQRELVAGISSDGSKVYIDSVKGRFTISRDNAKNSVYLQMNS |
| (SEQ ID NO: 399) | LRAEDTAVYYCYYFRTVSGSSYRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYSDSVKGRFTISRDNAKNSVYLQMNS |
| (SEQ ID NO: 400) | LRAEDTAVYYCYYFRTVLGSSMRYWGQGTLVTVSS |
| Exemplary anti-target | EVQLVESGGGLVQPGGSLTLSCVASGSTSIINAMGWYRRAPG |
| (DLL3) | KQRELAAGISSDGSKVIADSVKGRFTISRDNAKNSVYLQMNS |
| (SEQ ID NO: 401) | LRAEDTAVYYCYYFRRVSGSSMRYWGQGTLVTVSS |
| Exemplary anti-target | EVQLVESGGGLVQPGGSLTLSCVASGSTSSINAMGWYRRAPG |
| (DLL3) | KQRELVAGISSDGSKIYADSVKGRFTISRDNAKNSVYLQMNS |
| (SEQ ID NO: 402) | LRAEDTAVYYCYYFRTVSGSSMRYWGQGTLVTVSS |
| Exemplary anti-target | EVQLVESGGGLVQPGGSLTLSCVASGKTSSINAMAWYRRAPG |
| (DLL3) | KQRELVAGISSDGSKVYTDSVKGRFTISRDNAKNSVYLQMNS |
| (SEQ ID NO: 403) | LRAEDTAVYYCYYFRTVSGSSARYWGQGTLVTVSS |
| Exemplary anti-target | EVQLVESGGGLVQPGGSLTLSCVASGTTSSINAMGWYRRAPG |
| (DLL3) | KQRELVAGISSDGSLVYADSVKGRFTISRDNAKNSVYLQMNS |
| (SEQ ID NO: 404) | LRAEDTAVYYCYYFRIVRGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 405) | LRAEDTAVYYCYYYRTVSGSSMRYWGQGTLVTVSS |
| Exemplary anti-target | EVQLVESGGGLVQPGGSLTLSCVASGSGSSINAMGWYRRAPG |
| (DLL3) | KQRELVAGISSDGSKVYSDSVKGRFTISRDNAKNSVYLQMNS |
| (SEQ ID NO: 406) | LRAEDTAVYYCYYFRHVSGSSMRYWGQGTLVTVSS |
| Exemplary anti-target | EVQLVESGGGLVQPGGSLTLSCVASGTTSSINAMGWYRRAPG |
| (DLL3) | KQRELVAGISSDGSKVYVDSVKGRFTISRDNAKNSVYLQMNS |
| (SEQ ID NO: 407) | LRAEDTAVYYCYYFRFVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYVDSVKGRFTISRDNAKNSVYLQMNS |
| (SEQ ID NO: 408) | LRAEDTAVYYCYYFRTVSGSSMRYWGQGTLVTVSS |
| Exemplary anti-target | EVQLVESGGGLVQPGGSLTLSCVASGSTSRINAMGWYRRAPG |
| (DLL3) | KQRELVAGISSDGSKVYADSVKGRFTISRDNAKNSVYLQMNS |
| (SEQ ID NO: 409) | LRAEDTAVYYCYYFRTKSGSSMRYWGQGTLVTVSS |
| Exemplary anti-target | EVQLVESGGGLVQPGGSLTLSCVASGSTSRINAMGWYRRAPG |
| (DLL3) | KQRELVAGISSDGSKVYADSVKGRFTISRDNAKNSVYLQMNS |
| (SEQ ID NO: 410) | LRAEDTAVYYCYYFRTVYGSSMRYWGQGTLVTVSS |
| Exemplary anti-target | EVQLVESGGGLVQPGGSLTLSCVASGSTSSINAMGWYRRAPG |
| (DLL3) | KQRELVAGISSDGSKVYRDSVKGRFTISRDNAKNSVYLQMNS |
| (SEQ ID NO: 411) | LRAEDTAVYYCYYFRTVSGSSMGYWGQGTLVTVSS |
| Exemplary anti-target | EVQLVESGGGLVQPGGSLTLSCVASGSTSSINAMAWYRRAPG |
| (DLL3) | KQRELVAGISSDGSKVYSDSVKGRFTISRDNAKNSVYLQMNS |
| (SEQ ID NO: 412) | LRAEDTAVYYCYYFRTVSGSSMRSWGQGTLVTVSS |
| Exemplary anti-target | EVQLVESGGGLVQPGGSLTLSCVASGSTSSINAMAWYRRAPG |
| (DLL3) | KQRELVAGISSDNSKVYADSVKGRFTISRDNAKNSVYLQMNS |
| (SEQ ID NO: 413) | LRAEDTAVYYCYYFRTVGGSSMRYWGQGTLVTVSS |
| Exemplary anti-target | EVQLVESGGGLVQPGGSLTLSCVASGNTSSINAMAWYRRAPG |
| (DLL3) | KQRELVAGISSDGSKVYADSVKGRFTISRDNAKNSVYLQMNS |
| (SEQ ID NO: 414) | LRAEDTAVYYCYYFRTVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 415) | LRAEDTAVYYCYYFRTVSGSHMRYWGQGTLVTVSS |
| Exemplary anti-target | EVQLVESGGGLVQPGGSLTLSCVASGSTSIINAMGWYRRAPG |
| (DLL3) | KQRELVAGISSDGSKVYEDSVKGRFTISRDNAKNSVYLQMNS |
| (SEQ ID NO: 416) | LRAEDTAVYYCYYFRAVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 417) | LRAEDTAVYYCYYFRTVSGSSMRYWGQGTLVTVSS |
| Exemplary anti-target | EVQLVESGGGLVQPGGSLTLSCVASGSTSRINAMGWYRRAPG |
| (DLL3) | KQRELPAGISSDGSKVYAVSVKGRFTISRDNAKNSVYLQMNS |
| (SEQ ID NO: 418) | LRAEDTAVYYCYYFRTVSGSPMRYWGQGTLVTVSS |
| Exemplary anti-target | EVQLVESGGGLVQPGGSLTLSCVASGSTSRINAMGWYRRAPG |
| (DLL3) | KQRELVAGVSSDGSKVYADSVKGRFTISRDNAKNSVYLQMNS |
| (SEQ ID NO: 419) | LRAEDTAVYYCYYFRTVSGSSMSYWGQGTLVTVSS |
| Exemplary anti-target | EVQLVESGGGLVQPGGSLTLSCVASGTTSSINAMGWYRRAPG |
| (DLL3) | KQRELVAGISSDGSKVYEDSVKGRFTISRDNAKNSVYLQMNS |
| (SEQ ID NO: 420) | LRAEDTAVYYCYYFRTVSGSSMRYWGQGTLVTVSS |
| Exemplary anti-target | EVQLVESGGGLVQPGGSLTLSCVASGITSSINAMGWYRRAPG |
| (DLL3) | KQRELVAGISSDGSKVYAGSVKGRFTISRDNAKNSVYLQMNS |
| (SEQ ID NO: 421) | LRAEDTAVYYCYYFRTVRGSSMRYWGQGTLVTVSS |
| Exemplary anti-target | EVQLVESGGGLVQPGGSLTLSCVASGSTSDINAMGWYRRAPG |
| (DLL3) | KQRELVAGISSDKSKVYADSVKGRFTISRDNAKNSVYLQMNS |
| (SEQ ID NO: 422) | LRAEDTAVYYCYYFRTVRGSSMRYWGQGTLVTVSS |
| Exemplary anti-target | EVQLVESGGGLVQPGGSLTLSCVASGSTSSINAMAWYRRAPG |
| (DLL3) | KQRELVAGISSNGSKVYADSVKGRFTISRDNAKNSVYLQMNS |
| (SEQ ID NO: 423) | LRAEDTAVYYCYYFRQVSGSSMRYWGQGTLVTVSS |
| Exemplary anti-target | EVQLVESGGGLVQPGGSLTLSCVASGSTSSINAIGWYRRAPG |
| (DLL3) | KQRELVAGISSDGSKVLADSVKGRFTISRDNAKNSVYLQMNS |
| (SEQ ID NO: 424) | LRAEDTAVYYCYYFRIVSGSSMGYWGQGTLVTVSS |
| Exemplary anti-target | EVQLVESGGGLVQPGGSLTLSCVASGSTSSKNAMGWYRRAPG |
| (DLL3) | KQRELVAGISSDGSKVYADSVKGRFTISRDNAKNSVYLQMNS |
| (SEQ ID NO: 425) | LRAEDTAVYYCYYFRTVSGASMRYWGQGTLVTVSS |
| Exemplary anti-target | EVQLVESGGGLVQPGGSLTLSCVASGSTSSINAMGWYRRAPG |
| (DLL3) | KQRELVAGISSDNSKVYADSVKGRFTISRDNAKNSVYLQMNS |
| (SEQ ID NO: 426) | LRAEDTAVYYCYYFRTVHGSSMRYWGQGTLVTVSS |
| Exemplary anti-target | EVQLVESGGGLVQPGGSLTLSCVASGLTSSINAMGWYRRAPG |
| (DLL3) | KQRELVAGISSDGSKVYADSVKGRFTISRDNAKNSVYLQMNS |
| (SEQ ID NO: 427) | LRAEDTAVYYCYYFRMVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYTDSVKGRFTISRDNAKNSVYLQMNS |
| (SEQ ID NO: 428) | LRAEDTAVYYCYYFRTISGSSMRYWGQGTLVTVSS |
| Exemplary anti-target | EVQLVESGGGLVQPGGSLTLSCVASGSTSSNNAMAWYRRAPG |
| (DLL3) | KQRELVAGISSDGSKVYTDSVKGRFTISRDNAKNSVYLQMNS |
| (SEQ ID NO: 429) | LRAEDTAVYYCYYFRTRSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDNSKVYADSVKGRFTISRDNAKNSVYLQMNS |
| (SEQ ID NO: 430) | LRAEDTAVYYCYYFRTVSGHSMRYWGQGTLVTVSS |
| Exemplary anti-target | EVQLVESGGGLVQPGGSLTLSCVASGSTSHINAMGWYRRAPG |
| (DLL3) | KQRELVAGISSDGSRVYADSVKGRFTISRDNAKNSVYLQMNS |
| (SEQ ID NO: 431) | LRAEDTAVYYCYYFRTVSGGSMRYWGQGTLVTVSS |
| Exemplary anti-target | EVQLVESGGGLVQPGGSLTLSCVASGQTSSINAMGWYRRAPG |
| (DLL3) | KQRELVAGISSDGSQVYADSVKGRFTISRDNAKNSVYLQMNS |
| (SEQ ID NO: 432) | LRAEDTAVYYCYYFRTKSGSSMRYWGQGTLVTVSS |
| Exemplary anti-target | EVQLVESGGGLVQPGGSLTLSCVASGSTSRINGMGWYRRAPG |
| (DLL3) | KQRELPAGISSDGSKAYADSVKGRFTISRDNAKNSVYLQMNS |
| (SEQ ID NO: 433) | LRAEDTAVYYCYYFRTASGTSMRYWGQGTLVTVSS |
| Exemplary anti-target | EVQLVESGGGLVQPGGSLTLSCVASGSTSVINAMAWYRRAPG |
| (DLL3) | KQRELAAGISSDGSKVYAKSAKGRFTISRDNAKNSVYLQMNS |
| (SEQ ID NO: 434) | LRAEDTAVYYCYYFNTVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYNDSVKGRFTISRDNAKNSVYLQMNS |
| (SEQ ID NO: 435) | LRAEDTAVYYCYYFRTVRGSSQRYWGQGTLVTVSS |
| Exemplary anti-target | EVQLVESGGGLVQPGGSLTLSCVASGKTSSINAMGWYRRAPG |
| (DLL3) | KQRELVAGISSDGSKVIADSVKGRFTISRDNAKNSVYLQMNS |
| (SEQ ID NO: 436) | LRAEDTAVYYCYYFRTVLGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYTDSVKGRFTISRDNAKNSVYLQMNS |
| (SEQ ID NO: 437) | LRAEDTAVYYCYYFRTRSGSSMRYWGQGTLVTVSS |
| Exemplary anti-target | EVQLVESGGGLVQPGGSLTLSCVASGSVSSINAMGWYRRAPG |
| (DLL3) | KQRELVAGISSDGSKVYIDSVKGRFTISRDNAKNSVYLQMNS |
| (SEQ ID NO: 438) | LRAEDTAVYYCYYFRTVSGLSMRYWGQGTLVTVSS |
| Exemplary anti-target | EVQLVESGGGLVQPGGSLTLSCVASGNTSSINAMGWYRRAPG |
| (DLL3) | KQRELVAGISSDGSKVYYDSVKGRFTISRDNAKNSVYLQMNS |
| (SEQ ID NO: 439) | LRAEDTAVYYCYYFRTVRGSSQRYWGQGTLVTVSS |
| Exemplary anti-target | EVQLVESGGGLVQPGGSLTLSCVASGSTSSTNAMGWYRRAPG |
| (DLL3) | KQRELVAGISSDGSKVYVDSVKGRFTISRDNAKNSVYLQMNS |
| (SEQ ID NO: 440) | LRAEDTAVYYCYYFRTVSGSSMVYWGQGTLVTVSS |
| Exemplary anti-target | EVQLVESGGGLVQPGGSLTLSCVASGSTSSINAMGWYRRAPG |
| (DLL3) | KQRELVAGISSDGSKVYGDSVKGRFTISRDNAKNSVYLQMNS |
| (SEQ ID NO: 441) | LRAEDTAVYYCYYFRTVSRSSMRYWGQGTLVTVSS |
| Exemplary anti-target | EVQLVESGGGLVQPGGSLTLSCVASGSTSSINAMAWYRRAPG |
| (DLL3) | KQRELAAGISSDQSKVYADSAKGRFTISRDNAKNSVYLQMNS |
| (SEQ ID NO: 442) | LRAEDTAVYYCYYFRTVSGSSMSYWGQGTLVTVSS |
| Exemplary anti-target | EVQLVESGGGLVQPGGSLTLSCVASGGTSSINAMGWYRRAPG |
| (DLL3) | KQRELVAGISSDGSKVYSDSVKGRFTISRDNAKNSVYLQMNS |
| (SEQ ID NO: 443) | LRAEDTAVYYCYYFRTVSGSSARYWGQGTLVTVSS |
| Exemplary anti-target | EVQLVESGGGLVQPGGSLTLSCVASGSTRSINAMGWYRRAPG |
| (DLL3) | KQRELVAGISSDGSKVYADSVKGRFTISRDNAKNSVYLQMNS |
| (SEQ ID NO: 444) | LRAEDTAVYYCYYFHTVSGSSMRYWGQGTLVTVSS |
| Exemplary anti-target | EVQLVESGGGLVQPGGSLTLSCVASGTTSSINAMGWYRRAPG |
| (DLL3) | KQRELVAGISSDGSKVIADSVKGRFTISRDNAKNSVYLQMNS |
| (SEQ ID NO: 445) | LRAEDTAVYYCYYFRTVLGSSMRYWGQGTLVTVSS |
| Exemplary anti-target | EVQLVESGGGLVQPGGSLTLSCVASGSTSSINAMGWYRRAPG |
| (DLL3) | KQRELVAGISSDGSKVDADSVKGRFTISRDNAKNSVYLQMNS |
| (SEQ ID NO: 446) | LRAEDTAVYYCYYFRTVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYKDSVKGRFTISRDNAKNSVYLQMNS |
| (SEQ ID NO: 447) | LRAEDTAVYYCYYFRNVSGSSMRYWGQGTLVTVSS |
| Exemplary anti-target | EVQLVESGGGLVQPGGSLTLSCVASGNTSSINAMGWYRRAPG |
| (DLL3) | KQRELVAGISSNGSKVYADSVKGRFTISRDNAKNSVYLQMNS |
| (SEQ ID NO: 448) | LRAEDTAVYYCYYFRTVTGSSMRYWGQGTLVTVSS |
| Exemplary anti-target | EVQLVESGGGLVQPGGSLTLSCVASGSTSRINAMGWYRRAPG |
| (DLL3) | KQRELVAGISSDGSKVYKDSVKGRFTISRDNAKNSVYLQMNS |
| (SEQ ID NO: 449) | LRAEDTAVYYCYYFRTVSGSSMRYWGQGTLVTVSS |
| Exemplary anti-target | EVQLVESGGGLVQPGGSLTLSCVASGSTSSINAIGWYRRAPG |
| (DLL3) | KQRELVAGISSDGSKVYADSVKGRFTISRDNAKNSVYLQMNS |
| (SEQ ID NO: 450) | LRAEDTAVYYCYYFRTVKGSSMRYWGQGTLVTVSS |
| Exemplary anti-target | EVQLVESGGGLVQPGGSLTLSCVASGLTSSINAMGWYRRAPG |
| (DLL3) | KQRELVAGISSDGSKVYQDSVKGRFTISRDNAKNSVYLQMNS |
| (SEQ ID NO: 451) | LRAEDTAVYYCYYFRTNSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYAESVKGRFTISRDNAKNSVYLQMNS |
| (SEQ ID NO: 452) | LRAEDTAVYYCYYFRTVSGASMRYWGQGTLVTVSS |
| Exemplary anti-target | EVQLVESGGGLVQPGGSLTLSCVASGSTSSTNAMGWYRRAPG |
| (DLL3) | KQRELVAGISSDGSKVLADSVKGRFTISRDNAKNSVYLQMNS |
| (SEQ ID NO: 453) | LRAEDTAVYYCYYFRTVNLSSMRYWGQGTLVTVSS |
| Exemplary anti-target | EVQLVESGGGLVQPGGSLTLSCVASGSTSSINAIGWYRRAPG |
| (DLL3) | KQRELVAGISSDGSKYYADSVKGRFTISRDNAKNSVYLQMNS |
| (SEQ ID NO: 454) | LRAEDTAVYYCYYFRTVTGSSMRYWGQGTLVTVSS |
| Exemplary anti-target | EVQLVESGGGLVQPGGSLTLSCVASGSTSSINAIGWYRRAPG |
| (DLL3) | KQRELVAGISSDGSKVYAVSVKGRFTISRDNAKNSVYLQMNS |
| (SEQ ID NO: 455) | LRAEDTAVYYCYYFRKVSGSSARYWGQGTLVTVSS |
| Exemplary anti-target | EVQLVESGGGLVQPGGSLTLSCVASGSTSSINAMGWYRRAPG |
| (DLL3) | KQRELVAGISSDGSKVVADSVKGRFTISRDNAKNSVYLQMNS |
| (SEQ ID NO: 456) | LRAEDTAVYYCYYFRTYSGSSMRYWGQGTLVTVSS |
| Exemplary anti-target | EVQLVESGGGLVQPGGSLTLSCVASGSTSSINAMGWYRRAPG |
| (DLL3) | KQRELVAGISSDGSKVYADSVKGRFTISRDNAKNSVYLQMNS |
| (SEQ ID NO: 457) | LRAEDTAVYYCYYFRTVSKSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 458) | LRAEDTAVYYCYYFKTVSGSSMRYWGQGTLVTVSS |
| Exemplary anti-target | EVQLVESGGGLVQPGGSLTLSCVASGSTSSINAMAWYRRAPG |
| (DLL3) | KQRELAAGISSDNSKVYADSVKGRFTISRDNAKNSVYLQMNS |
| (SEQ ID NO: 459) | LRAEDTAVYYCYYFRTRSGSSMRYWGQGTLVTVSS |
| Exemplary anti-target | EVQLVESGGGLVQPGGSLTLSCVASGSKSSINAMGWYRRAPG |
| (DLL3) | KQRELAAGISSDGSKVYAQSVKGRFTISRDNAKNSVYLQMNS |
| (SEQ ID NO: 460) | LRAEDTAVYYCYYFRTSSGSSMRYWGQGTLVTVSS |
| Exemplary anti-target | EVQLVESGGGLVQPGGSLTLSCVASGTTSSINAMGWYRRAPG |
| (DLL3) | KQRELVAGISSDGSKVYVDSVKGRFTISRDNAKNSVYLQMNS |
| (SEQ ID NO: 461) | LRAEDTAVYYCYYFRFLSGSSMRYWGQGTLVTVSS |
| Exemplary anti-target | EVQLVESGGGLVQPGGSLTLSCVASGSTSSINAFGWYRRAPG |
| (DLL3) | KQRELVAGISSDGSKVYSDSVKGRFTISRDNAKNSVYLQMNS |
| (SEQ ID NO: 462) | LRAEDTAVYYCYYFRTVSGSSMRYWGQGTLVTVSS |
| Exemplary anti-target | EVQLVESGGGLVQPGGSLTLSCVASGSTESINAMGWYRRAPG |
| (DLL3) | KQRELVAGISSDGSKVLADSVKGRFTISRDNAKNSVYLQMNS |
| (SEQ ID NO: 463) | LRAEDTAVYYCYYFRLVSGSSMRYWGQGTLVTVSS |
| Exemplary anti-target | EVQLVESGGGLVQPGGSLTLSCVASGSTRSINAMGWYRRAPG |
| (DLL3) | KQRELVAGISSDGSKVYNDSVKGRFTISRDNAKNSVYLQMNS |
| (SEQ ID NO: 464) | LRAEDTAVYYCYYFRTVSGSSMRFWGQGTLVTVSS |
| Exemplary anti-target | EVQLVESGGGLVQPGGSLTLSCVASGSTSSINAIGWYRRAPG |
| (DLL3) | KQRELVAGISSDGSKVYNDSVKGRFTISRDNAKNSVYLQMNS |
| (SEQ ID NO: 465) | LRAEDTAVYYCYYFRTQSGSSMRYWGQGTLVTVSS |
| Exemplary anti-target | EVQLVESGGGLVQPGGSLTLSCVASGSTSSINAMGWYRRAPG |
| (DLL3) | KQRELVAGISSDGSKVYVDSVKGRFTISRDNAKNSVYLQMNS |
| (SEQ ID NO: 466) | LRAEDTAVYYCYYFRTVSGSSMPYWGQGTLVTVSS |
| Exemplary anti-target | EVQLVESGGGLVQPGGSLTLSCVASGSTSSINAMGWYRRAPG |
| (DLL3) | KQRELVAGISSDGSKVVADSVKGRFTISRDNAKNSVYLQMNS |
| (SEQ ID NO: 467) | LRAEDTAVYYCYYFRTLSGSSMRYWGQGTLVTVSS |
| Exemplary anti-target | EVQLVESGGGLVQPGGSLTLSCVASGTTSSINAMGWYRRAPG |
| (DLL3) | KQRELVAGISSDGSKVYGDSVKGRFTISRDNAKNSVYLQMNS |
| (SEQ ID NO: 468) | LRAEDTAVYYCYYFRTVSGSAMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYTDSVKGRFTISRDNAKNSVYLQMNS |
| (SEQ ID NO: 469) | LRAEDTAVYYCYYFRTTSGSSMRYWGQGTLVTVSS |
| Exemplary anti-target | EVQLVESGGGLVQPGGSLTLSCVASGRTSSINAMGWYRRAPG |
| (DLL3) | KQRELVAGISSDGSKVYNDSVKGRFTISRDNAKNSVYLQMNS |
| (SEQ ID NO: 470) | LRAEDTAVYYCYYFRTVSGTSMRYWGQGTLVTVSS |
| Exemplary anti-target | EVQLVESGGGLVQPGGSLTLSCVASGSTSSRNAMGWYRRAPG |
| (DLL3) | KQRELVAGISSDGSKVTADSVKGRFTISRDNAKNSVYLQMNS |
| (SEQ ID NO: 471) | LRAEDTAVYYCYYFRTRSGSSMRYWGQGTLVTVSS |
| Exemplary anti-target | EVQLVESGGGLVQPGGSLTLSCVASGSTKSINAMGWYRRAPG |
| (DLL3) | KQRELVAGISSDGSKVYRDSVKGRFTISRDNAKNSVYLQMNS |
| (SEQ ID NO: 472) | LRAEDTAVYYCYYFRTSSGSSMRYWGQGTLVTVSS |
| Exemplary anti-target | EVQLVESGGGLVQPGGSLTLSCVASGSTSSRNAMGWYRRAPG |
| (DLL3) | KQRELVAGISSNGSKVYSDSVKGRFTISRDNAKNSVYLQMNS |
| (SEQ ID NO: 473) | LRAEDTAVYYCYYFRTVSGSSMSYWGQGTLVTVSS |
| Exemplary anti-target | EVQLVESGGGLVQPGGSLTLSCVASGSTSSINAIGWYRRAPG |
| (DLL3) | KQRELVAGISSDGSKVYSDSVKGRFTISRDNAKNSVYLQMNS |
| (SEQ ID NO: 474) | LRAEDTAVYYCYYFRPVSGSSMRYWGQGTLVTVSS |
| Exemplary anti-target | EVQLVESGGGLVQPGGSLTLSCVASGSTSLINAMGWYRRAPG |
| (DLL3) | KQRELVAGISSDGSKVYADSVKGRFTISRDNAKNSVYLQMNS |
| (SEQ ID NO: 475) | LRAEDTAVYYCYYFRHVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 476) | LRAEDTAVYYCYYFRTKSGSSMRYWGQGTLVTVSS |
| Exemplary anti-target | EVQLVESGGGLVQPGGSLTLSCVASGSTSSINAMGWYRRAPG |
| (DLL3) | KQRELVAGISSDGSLVYADSVKGRFTISRDNAKNSVYLQMNS |
| (SEQ ID NO: 477) | LRAEDTAVYYCYYFTTVSGSSMRYWGQGTLVTVSS |
| Exemplary anti-target | EVQLVESGGGLVQPGGSLTLSCVASGTTSSINAMGWYRRAPG |
| (DLL3) | KQRELVAGISSDGTKVYADSVKGRFTISRDNAKNSVYLQMNS |
| (SEQ ID NO: 478) | LRAEDTAVYYCYYFHTVSGSSMRYWGQGTLVTVSS |
| Exemplary anti-target | EVQLVESGGGLVQPGGSLTLSCVASGSTSSINAFGWYRRAPG |
| (DLL3) | KQRELVAGISSDGSKVYADSVKGRFTISRDNAKNSVYLQMNS |
| (SEQ ID NO: 479) | LRAEDTAVYYCYYFRTVRGSSMRYWGQGTLVTVSS |
| Exemplary anti-target | EVQLVESGGGLVQPGGSLTLSCVASGSTSSRNAMGWYRRAPG |
| (DLL3) | KQRELVAGISSDGSKLYLDSVKGRFTISRDNAKNSVYLQMNS |
| (SEQ ID NO: 480) | LRAEDTAVYYCYYFRTVLGSSMRYWGQGTLVTVSS |
| Exemplary anti-target | EVQLVESGGGLVQPGGSLTLSCVASGNTSSINAMGWYRRAPG |
| (DLL3) | KQRELVAGISSDGSRVYADSVKGRFTISRDNAKNSVYLQMNS |
| (SEQ ID NO: 481) | LRAEDTAVYYCYYFRTVSGSSMRSWGQGTLVTVSS |
| Exemplary anti-target | EVQLVESGGGLVQPGGSLTLSCVASGTTSSINAMGWYRRAPG |
| (DLL3) | KQRELVAGISSDGSKVYNDSVKGRFTISRDNAKNSVYLQMNS |
| (SEQ ID NO: 482) | LRAEDTAVYYCYYFRTVRGSSMRYWGQGTLVTVSS |
| Exemplary anti-target | EVQLVESGGGLVQPGGSLTLSCVASGSTASINAMGWYRRAPG |
| (DLL3) | KQRELVAGISSDGSKVYADSVKGRFTISRDNAKNSVYLQMNS |
| (SEQ ID NO: 483) | LRAEDTAVYYCYYFRYVSGSSMRYWGQGTLVTVSS |
| Exemplary anti-target | EVQLVESGGGLVQPGGSLTLSCVASGSTSSINAVGWYRRAPG |
| (DLL3) | KQRELVAGISSDGSKVYVDSVKGRFTISRDNAKNSVYLQMNS |
| (SEQ ID NO: 484) | LRAEDTAVYYCYYFRTVYGSSMRYWGQGTLVTVSS |
| Exemplary anti-target | EVQLVESGGGLVQPGGSLTLSCVASGSTSSRNAMGWYRRAPG |
| (DLL3) | KQRELVAGISSDGSKLYADSVKGRFTISRDNAKNSVYLQMNS |
| (SEQ ID NO: 485) | LRAEDTAVYYCYYFRTVLGSSMRYWGQGTLVTVSS |
| Exemplary anti-target | EVQLVESGGGLVQPGGSLTLSCVASGSTNSINAMGWYRRAPG |
| (DLL3) | KQRELVAGISSDGSKVYKDSVKGRFTISRDNAKNSVYLQMNS |
| (SEQ ID NO: 486) | LRAEDTAVYYCYYYRTVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 487) | LRAEDTAVYYCYYFRSVSGSSMRYWGQGTLVTVSS |
| Exemplary anti-target | EVQLVESGGGLVQPGGSLTLSCVASGSTSRINAMGWYRRAPG |
| (DLL3) | KQRELVAGISSDGSKVYQDSVKGRFTISRDNAKNSVYLQMNS |
| (SEQ ID NO: 488) | LRAEDTAVYYCYYFRRVSGSSMRYWGQGTLVTVSS |
| Exemplary anti-target | EVQLVESGGGLVQPGGSLTLSCVPSGSTSNINAMGWYRRAPG |
| (DLL3) | KQRELPAGISSDGTKIYADSAKVPFTITRDNAKNSVYLQMNS |
| (SEQ ID NO: 489) | LRAEDTAVYYCYYFRTVSGTSMRYWGQGTLVTVSS |
| Exemplary anti-target | EVQLVESGGGLVQPGGSLTLSCVASGSTSKINAMGWYRRAPG |
| (DLL3) | KQRELVAGISSDRSKVYADSVKGRFTISRDNAKNSVYLQMNS |
| (SEQ ID NO: 490) | LRAEDTAVYYCYYFRTVAGSSMRYWGQGTLVTVSS |
| Exemplary anti-target | EVQLVESGGGLVQPGGSLTLSCVASGSTSSINALGWYRRAPG |
| (DLL3) | KQRELVAGISSDGSLVYADSVKGRFTISRDNAKNSVYLQMNS |
| (SEQ ID NO: 491) | LRAEDTAVYYCYYFRIVSGSSMRYWGQGTLVTVSS |
| Exemplary anti-target | EVQLVESGGGLVQPGGSLTLSCVASGKTSSINAMGWYRRAPG |
| (DLL3) | KQRELVAGISSDGSKVYADSVKGRFTISRDNAKNSVYLQMNS |
| (SEQ ID NO: 490) | LRAEDTAVYYCYYFRTVSGVSMRYWGQGTLVTVSS |
| Exemplary anti-target | EVQLVESGGGLVQPGGSLTLSCVASGSTSSINAVGWYRRAPG |
| (DLL3) | KQRELVAGISSDGSKVYRDSVKGRFTISRDNAKNSVYLQMNS |
| (SEQ ID NO: 491) | LRAEDTAVYYCYYFRTVQGSSMRYWGQGTLVTVSS |
| Exemplary anti-target | EVQLVESGGGLVQPGGSLTLSCVASGSTSRINAMGWYRRAPG |
| (DLL3) | KQRELVAGISSDGSKVYADSVKGRFTISRDNAKNSVYLQMNS |
| (SEQ ID NO: 492) | LRAEDTAVYYCYYFRTASGSSMRYWGQGTLVTVSS |
| Exemplary anti-target | EVQLVESGGGLVQPGGSLTLSCVASGSTSSINAMGWYRRAPG |
| (DLL3) | KQRELVAGISSDGSKVYSDSVKGRFTISRDNAKNSVYLQMNS |
| (SEQ ID NO: 493) | LRAEDTAVYYCYYFRTVSGSSSRYWGQGTLVTVSS |
| Exemplary anti-target | EVQLVESGGGLVQPGGSLTLSCVASGSTSSINAVGWYRRAPG |
| (DLL3) | KQRELVAGISSDGTKVYRDSVKGRFTISRDNAKNSVYLQMNS |
| (SEQ ID NO: 494) | LRAEDTAVYYCYYFRTVQGSSMRYWGQGTLVTVSS |
| Exemplary anti-target | EVQLVESGGGLVQPGGSLTLSCVASGTTSSINAMGWYRRAPG |
| (DLL3) | KQRELAAGISSDGSKVYNDSVKGRFTISRDNAKNSVYLQMNS |
| (SEQ ID NO: 495) | LRAEDTAVYYCYYFRTVRGSSMRYWGQGTLVTVSS |
| Exemplary anti-target | EVQLVESGGGLVQPGGSLTLSCVASGSTSSINAMGWYRRAPG |
| (DLL3) | KQRELVAGISSDGSKVYADSVKGRFTISRDNAKNSVYLQMNS |
| (SEQ ID NO: 496) | LRAEDTAVYYCYYFRTKSGSSMRYWGQGTLVTVSS |
| Exemplary anti-target | EVQLVESGGGLVQPGGSLTLSCVASGTTSSINAMGWYRRAPG |
| (DLL3) | KQRELVAGISSDGSKVYADSVKGRFTISRDNAKNSVYLQMNS |
| (SEQ ID NO: 497) | LRAEDTAVYYCYYFRTVWGSSMRYWGQGTLVTVSS |
| Exemplary anti-target | EVQLVESGGGLVQPGGSLTLSCVASGKTSSINAMGWYRRAPG |
| (DLL3) | KQRELVAGISSDGSKVYTDSVKGRFTISRDNAKNSVYLQMNS |
| (SEQ ID NO: 498) | LRAEDTAVYYCYYFRTRSGSSMRYWGQGTLVTVSS |
| Exemplary anti-target | EVQLVESGGGLVQPGGSLTLSCVASGSTSSINAMGWYRRAPF |
| (DLL3) | KQGELPAGISPDGTKAYADSAKVRFTISRDNAKNSVYLQMNS |
| (SEQ ID NO: 499) | LRAEDTAVYYCYYFHTVCGTSMGYWGQGTLVTVSS |
| Exemplary anti-target | EVQLVESGGGLVQPGGSLTLSCVASGSTSAINAMGWYRRAPG |
| (DLL3) | KQRELVAGISSDGSKVYVDSVKGRFTISRDNAKNSVYLQMNS |
| (SEQ ID NO: 500) | LRAEDTAVYYCYYFRTVSGSSQRYWGQGTLVTVSS |
| Exemplary anti-target | EVQLVESGGGLVQPGGSLTLSCVASGSPSSINAYGWYRRAPG |
| (DLL3) | KQRELVAGISSDGSKVYSDSVKGRFTISRDNAKNSVYLQMNS |
| (SEQ ID NO: 501) | LRAEDTAVYYCYYFRTVSGSSMSYWGQGTLVTVSS |
| Exemplary anti-target | EVQLVESGGGLVQPGGSLTLSCVASGSTSSINAMGWYRRAPG |
| (DLL3) | KQRELVAGISSDGSKVYASSVKGRFTISRDNAKNSVYLQMNS |
| (SEQ ID NO: 502) | LRAEDTAVYYCYYFRTVRGSSMRYWGQGTLVTVSS |
| Exemplary anti-target | EVQLVESGGGLVQPGGSLTLSCVASGSRSSINAMGWYRRAPG |
| (DLL3) | KQRELVAGISADGSKVYADSVKGRFTISRDNAKNSVYLQMNS |
| (SEQ ID NO: 503) | LRAEDTAVYYCYYFRTQSGSSMRYWGQGTLVTVSS |
| Exemplary anti-target | EVQLVESGGGLVQPGGSLTLSCVASGSVSSINAMGWYRRAPG |
| (DLL3) | KQRELVAGISSDGSKVYASSAKGRFTISRDNAKNSVYLQMNS |
| (SEQ ID NO: 504) | LRAEDTAVYYCYYFRTLSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 505) | LRAEDTAVYYCYYFHTVSGSSMRYWGQGTLVTVSS |
| Exemplary anti-target | EVQLVESGGGLVQPGGSLTLSCVASGSTSSINAIGWYRRAPG |
| (DLL3) | KQRELVAGISSDGSSVYADSVKGRFTISRDNAKNSVYLQMNS |
| (SEQ ID NO: 506) | LRAEDTAVYYCYYFRRVSGSSMRYWGQGTLVTVSS |
| Exemplary anti-target | EVQLVESGGGLVQPGGSLTLSCVASGSTSSINAMGWYRRAPG |
| (DLL3) | KQRELVAGISSDGSKVYSDSVKGRFTISRDNAKNSVYLQMNS |
| (SEQ ID NO: 507) | LRAEDTAVYYCYYFRLVSGSSMRYWGQGTLVTVSS |
| Exemplary anti-target | EVQLVESGGGLVQPGGSLTLSCVASGSTSSINAVGWYRRAPG |
| (DLL3) | KQRELVAGISSDGSKVYAGSVKGRFTISRDNAKNSVYLQMNS |
| (SEQ ID NO: 508) | LRAEDTAVYYCYYFRTVSGSYMRYWGQGTLVTVSS |
| Exemplary anti-target | EVQLVESGGGLVQPGGSLTLSCVASGSTSSINAMAWYRRAPG |
| (DLL3) | KQRELAAGISSDNSKVYADSVKGRFTISRDNAKNSVYLQMNS |
| (SEQ ID NO: 509) | LRAEDTAVYYCYYFRTVGGSSMRYWGQGTLVTVSS |
| Exemplary anti-target | EVQLVESGGGLVQPGGSLTLSCVASGSTSSINAYGWYRRAPG |
| (DLL3) | KQRELVAGISSDGSAVYADSVKGRFTISRDNAKNSVYLQMNS |
| (SEQ ID NO: 510) | LRAEDTAVYYCYYFRTHSGSSMRYWGQGTLVTVSS |
| Exemplary anti-target | EVQLVESGGGLVQPGGSLTLSCVASGSTSSINAVGWYRRAPG |
| (DLL3) | KQRELVAGISSDGSSVYADSVKGRFTISRDNAKNSVYLQMNS |
| (SEQ ID NO: 511) | LRAEDTAVYYCYYFRTVSTSSMRYWGQGTLVTVSS |
| Exemplary anti-target | EVQLVESGGGLVQPGGSLTLSCVASGSKSSINAMGWYRRAPG |
| (DLL3) | KQRELPAGISSNGTKVYADSAKVRETISRDNAKNSVYLQMNS |
| (SEQ ID NO: 512) | LRAEDTAVYYCYYFRTVLGTSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKLYADSVKGRFTISRDNAKNSVYLQMNS |
| (SEQ ID NO: 513) | LRAEDTAVYYCYYFRTVSGSSMRYWGQGTLVTVSS |
| Exemplary anti-target | EVQLVESGGGLVQPGGSLTLSCVASGSVSSINAMGWYRRAPG |
| (DLL3) | KQRELVAGISSDGSKVYKDSVKGRFTISRDNAKNSVYLQMNS |
| (SEQ ID NO: 514) | LRAEDTAVYYCYYFRTVSGSSMGYWGQGTLVTVSS |
| Exemplary anti-target | EVQLVESGGGLVQPGGSLTLSCVASGSTSSINAMGWYRRAPG |
| (DLL3) | KQRELVAGISSDGSLVYADSVKGRFTISRDNAKNSVYLQMNS |
| (SEQ ID NO: 515) | LRAEDTAVYYCYYFRTVSGSSMRAWGQGTLVTVSS |
| Exemplary anti-target | EVQLVESGGGLVQPGGSLTLSCVASGSTSSINAMGWYRRAPG |
| (DLL3) | KQRELVAGISSDGSLVYADSVKGRFTISRDNAKNSVYLQMNS |
| (SEQ ID NO: 516) | LRAEDTAVYYCYYFRILSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 517) | LRAEDTAVYYCYYFRTVQGSSMRYWGQGTLVTVSS |
| Exemplary anti-target | EVQLVESGGGLVQPGGSLTLSCVASGSTSYINAMGWYRRAPG |
| (DLL3) | KQRELVAGISSDGSKVYADSVKGRFTISRDNAKNSVYLQMNS |
| (SEQ ID NO: 518) | LRAEDTAVYYCYYFRTVSGQSMGYWGQGTLVTVSS |
| Exemplary anti-target | EVQLVESGGGLVQPGGSLTLSCVASGSTSSINAMGWYRRAPG |
| (DLL3) | KQRELVAGVSSDGSKVYADSVKGRFTISRDNAKNSVYLQMNS |
| (SEQ ID NO: 511) | LRAEDTAVYYCYYFRTVSGSSARYWGQGTLVTVSS |
| Exemplary anti-target | EVQLVESGGGLVQPGGSLTLSCVASGSTSSINAMGWYRRAPG |
| (DLL3) | KQRELPAGISRDGSKVYADSVKGRFTISRDNAKNSVYLQMNS |
| (SEQ ID NO: 512) | LRAEDTAVYYCYYFRYVSGSSMRYWGQGTLVTVSS |
| Exemplary anti-target | EVQLVESGGGLVQPGGSLTLSCVASGSTSSINAMGWYRRAPG |
| (DLL3) | KQRELAAGISSDGSKLYADSVKGRFTISRDNAKNSVYLQMNS |
| (SEQ ID NO: 513) | LRAEDTAVYYCYYFRTVSGSSMRYWGQGTLVTVSS |
| Exemplary anti-target | EVQLVESGGGLVQPGGSLTLSCVASGSTSRINAMGWYRRAPG |
| (DLL3) | KQRELVAGISSDGSKVYADSVKGRFTISRDNAKNSVYLQMNS |
| (SEQ ID NO: 514) | LRAEDTAVYYCYYFRRVSGSSMRYWGQGTLVTVSS |
| Exemplary anti-target | EVQLVESGGGLVQPGGSLTLSCVASGSTSSINAMAWYRRAPG |
| (DLL3) | KQRELAAGISSDGSKVYADSVKGRFTISRDNAKNSVYLQMNS |
| (SEQ ID NO: 515) | LRAEDTAVYYCYYFHTVSGSSMRYWGQGTLVTVSS |
| Exemplary anti-target | EVQLVESGGGLVQPGGSLTLSCVASGSTSSINAMAWYRRAPG |
| (DLL3) | KQRELVAGISSDGSKVYADSVKGRFTISRDNAKNSVYLQMNS |
| (SEQ ID NO: 516) | LRAEDTAVYYCYYFRQVSGSSMRYWGQGTLVTVSS |
| Exemplary anti-target | EVQLVESGGGLVQPGGSLTLSCVASGSTSSINAMGWYRRAPG |
| (DLL3) | KQRELVAGISSDTSKVYADSVKGRFTISRDNAKNSVYLQMNS |
| (SEQ ID NO: 517) | LRAEDTAVYYCYYFRTVSGSYMRYWGQGTLVTVSS |
| Exemplary anti-target | EVQLVESGGGLVQPGGSLTLSCVASGSTSTINAMGWYRRAPG |
| (DLL3) | KQRELVAGISSDGSKVYADSVKGRFTISRDNAKNSVYLQMNS |
| (SEQ ID NO: 518) | LRAEDTAVYYCYYFRTASGSSMRYWGQGTLVTVSS |
| Exemplary anti-target | EVQLVESGGGLVQPGGSLTLSCVASGSTSSINAVGWYRRAPG |
| (DLL3) | KQRELVAGISSDGSTVYADSVKGRFTISRDNAKNSVYLQMNS |
| (SEQ ID NO: 519) | LRAEDTAVYYCYYFRTVSGHSMRYWGQGTLVTVSS |
| Exemplary anti-target | EVQLVESGGGLVQPGGSLTLSCVASGSTSSINAMGWYRRAPG |
| (DLL3) | KQRELAAGISKDGSKVYADSAKGRFTISRDNAKNSVYLQMNS |
| (SEQ ID NO: 520) | LRAEDTAVYYCYYFRTVSGSSSRYWGQGTLVTVSS |
| Exemplary anti-target | EVQLVESGGGLVQPGGSLTLSCVASGSPSSINAYGWYRRAPG |
| (DLL3) | KQRELVAGISSDGSKVYSDSVKGRFTISRDNAKNSVYLQMNS |
| (SEQ ID NO: 521) | LRAEDTAVYYCYYFRTVSGSSYSYWGQGTLVTVSS |
| Exemplary anti-target | EVQLVESGGGLVQPGGSLTLSCVASGSPSSINAYGWYRRAPG |
| (DLL3) | KQRELVAGISSDGSKVYSDSVKGRFTISRDNAKNSVYLQMNS |
| (SEQ ID NO: 522) | LRAEDTAVYYCYYFRTVSGSSQSYWGQGTLVTVSS |
| Exemplary anti-target | EVQLVESGGGLVQPGGSLTLSCAASGSSVKFLSMAWYRQAPG |
| (DLL3) | KKRELVAGISADGSTDYIDSVKGRFTISRDNAKNSVYLQMNS |
| (SEQ ID NO: 523) | LRAEDTAVYYCYAYRWTRRYTYWGQGTLVTVSS |
| Exemplary anti-target | EVQLVESGGGLVQPGGSLTLSCAASGSSVSFLSLAWYRQAPG |
| (DLL3) | KKRELVAGISADGSTAYIDSVKGRFTISRDNAKNSVYLQMNS |
| (SEQ ID NO: 524) | LRAEDTAVYYCYAYRWRTRYTYWGQGTLVTVSS |
| Exemplary anti-target | EVQLVESGGGLVQPGGSLTLSCAASGSRVSFLSMAWYRQAPG |
| (DLL3) | KKRELVAGISRDGSTDYIDSVKGRFTISRDNAKNSVYLQMNS |
| (SEQ ID NO: 525) | LRAEDTAVYYCYAYRWTTRRTYWGQGTLVTVSS |
| Exemplary anti-target | EVQLVESGGGLVQPGGSLTLSCAASGSQVSFLSMAWYRQAPG |
| (DLL3) | KKRELVAGISRDGSTDYIDSVKGRFTISRDNAKNSVYLQMNS |
| (SEQ ID NO: 526) | LRAEDTAVYYCYAYRWTTRYIYWGQGTLVTVSS |
| Exemplary anti-target | EVQLVESGGGLVQPGGSLTLSCAASGSSVSFLSMAWYRQAPG |
| (DLL3) | KKRELVAGISEAGSTDYIDSVKGRFTISRDNAKNSVYLQMNS |
| (SEQ ID NO: 527) | LRAEDTAVYYCYAYRWRTRYTYWGQGTLVTVSS |
| Exemplary anti-target | EVQLVESGGGLVQPGGSLTLRCAASGSKVSFLSMAWYRQAPG |
| (DLL3) | KKRELVAGISADGSTDYVDSVKGRFTISRDNAKNSVYLQMNS |
| (SEQ ID NO: 528) | LRAEDTAVYYCYAYRWTRRYTYWGQGTLVTVSS |
| Exemplary anti-target | EVQLVESGGGLVQPGGSLTLSCAASGSSVGFLSMAWYRQAPG |
| (DLL3) | KKRELVAGISADGSTDYIRSVKGRFTISRDNAKNSVYLQMNS |
| (SEQ ID NO: 529) | LRAEDTAVYYCYAYRWRTRYTYWGQGTLVTVSS |
| Exemplary anti-target | EVQLVESGGGLVQPGGSLTLSCAASGSSVSFLSMAWYRQAPG |
| (DLL3) | KKRELVAGISADGSVDYIDSVKGRFTISRDNAKNSVYLQMNS |
| (SEQ ID NO: 530) | LRAEDTAVYYCYAYRWTTRYIYWGQGTLVTVSS |
| Exemplary anti-target | EVQLVESGGGLVQPGGSLTLSCAASGSRVSFLSMAWYRQAPG |
| (DLL3) | KKRELVAGISADGSTLYIDSVKGRFTISRDNAKNSVYLQMNS |
| (SEQ ID NO: 531) | LRAEDTAVYYCYAYRWTTRRTYWGQGTLVTVSS |
| Exemplary anti-target | EVQLVESGGGLVQPGGSLTLSCAASGSSVSFLSLAWYRQAPG |
| (DLL3) | KKRELVAGISTDGSTDYIDSVKGRFTISRDNAKNSVYLQMNS |
| (SEQ ID NO: 532) | LRAEDTAVYYCYAYRWRTRYTYWGQGTLVTVSS |
| Exemplary anti-target | EVQLVESGGGLVQPGGSLTLSCAASGSSVSFLSMAWYRQAPG |
| (DLL3) | KKRELVAGISGDGSTDYIDSVKGRFTISRDNAKNSVYLQMNS |
| (SEQ ID NO: 533) | LRAEDTAVYYCYAYRWTTRYTYWGQGTLVTVSS |
| Exemplary anti-target | EVQLVESGGGLVQPGGSLTLSCAASGSSVQFLSMAWYRQAPG |
| (DLL3) | KKRELVAGISADGSTDYINSVKGRFTISRDNAKNSVYLQMNS |
| (SEQ ID NO: 534) | LRAEDTAVYYCYAYRWRTRYTYWGQGTLVTVSS |
| Exemplary anti-target | EVQLVESGGGLVQPGGSLTLSCAASGSNVSFLSMAWYRQAPG |
| (DLL3) | KKRELVAGISARGSTDYIDSVKGRFTISRDNAKNSVYLQMNS |
| (SEQ ID NO: 535) | LRAEDTAVYYCYAYHWTTRYTYWGQGTLVTVSS |
| Exemplary anti-target | EVQLVESGGGLVQPGGSLTLSCVASGSSVKFLSMAWYRQAPG |
| (DLL3) | KKRELVAGISADGSTTYIDSVKGRFTISRDNAKNSVYLQMNS |
| (SEQ ID NO: 536) | LRAEDTAVYYCYAYRWTTRRTYWGQGTLVTVSS |
| Exemplary anti-target | EVQLVESGGGLVQPGGSLTLSCAASGKSVSFLSMAWYRQAPG |
| (DLL3) | KKRELVAGISKDGSTDYIDSVKGRFTISRDNAKNSVYLQMNS |
| (SEQ ID NO: 537) | LRAEDTAVYYCYAYRWTTRYTYWGQGTLVTVSS |
| Exemplary anti-target | EVQLVESGGGLVQPGGSLTLSCAASGSRVSFLSMAWYRQAPG |
| (DLL3) | KKRELVAGISADGSTTYIDSVKGRFTISRDNAKNSVYLQMNS |
| (SEQ ID NO: 538) | LRAEDTAVYYCYAYRWTTRYTYWGQGTLVTVSS |
| Exemplary anti-target | EVQLVESGGGLVQPGGSLTLSCAASGSHVSFLSMAWYRQAPG |
| (DLL3) | KKRELVAGISANGSTDYIDSVKGRFTISRDNAKNSVYLQMNS |
| (SEQ ID NO: 539) | LRAEDTAVYYCYAYRWTTRYAYWGQGTLVTVSS |
| Exemplary anti-target | EVQLVESGGGLVQPGGSLTLSCAASGSSVSFLSMAWYRQAPG |
| (DLL3) | KKRELVAGISRDGSTDYIDSVKGRFTISRDNAKNSVYLQMNS |
| (SEQ ID NO: 540) | LRAEDTAVYYCYAYRWVTRYTYWGQGTLVTVSS |
| Exemplary anti-target | EVQLVESGGGLVQPGGSLTLSCAASGSSVSFLSMAWYRQAPG |
| (DLL3) | KKRELVAGISADGSADYIDSVKGRFTISRDNAKNSVYLQMNS |
| (SEQ ID NO: 541) | LRAEDTAVYYCYAYRWVTRYTYWGQGTLVTVSS |
| Exemplary anti-target | EVQLVESGGGLVQPGGSLTLSCAASGSSVRFLSMAWYRQAPG |
| (DLL3) | KKRELVAGISAHGSTDYIDSVKGRFTISRDNAKNSVYLQMNS |
| (SEQ ID NO: 542) | LRAEDTAVYYCYAYRWSTRYTYWGQGTLVTVSS |
| Exemplary anti-target | EVQLVESGGGLVQPGGSLTLSCAASGSSVRFLSMAWYRQAPG |
| (DLL3) | KKRELVAGISADGSTIYIDSVKGRFTISRDNAKNSVYLQMNS |
| (SEQ ID NO: 543) | LRAEDTAVYYCYAYRWRTRYTYWGQGTLVTVSS |
| Exemplary anti-target | EVQLVESGGGLVQPGGSLTLSCAASGSSVRFLSMAWYRQAPG |
| (DLL3) | KKRELVAGISRDGSTVYIDSVKGRFTISRDNAKNSVYLQMNS |
| (SEQ ID NO: 544) | LRAEDTAVYYCYAYRWTTRGTYWGQGTLVTVSS |
| Exemplary anti-target | EVQLVESGGGLVQPGGSLTLSCAASGSHVSFLSMAWYRQAPG |
| (DLL3) | KKRELVAGISADGPTDYIDSVKGRFTISRDNAKNSVYLQMNS |
| (SEQ ID NO: 545) | LRAEDTAVYYCYAYRWDTRYTYWGQGTLVTVSS |
| Exemplary anti-target | EVQLVESGGGLVQPGGSLTLSCVASGTSVSFLSMAWYRQAPG |
| (DLL3) | KKRELVAGISADGSTTYIDSVKGRFTISRDNAKNSVYLQMNS |
| (SEQ ID NO: 546) | LRAEDTAVYYCYAYRWTTRRTYWGQGTLVTVSS |
| Exemplary anti-target | EVQLVESGGGLVQPGGSLTLSCAASGTSVSFLSIAWYRQAPG |
| (DLL3) | KKRELVAGISADGSTDYIASVKGRFTISRDNAKNSVYLQMNS |
| (SEQ ID NO: 547) | LRAEDTAVYYCYAYRWRTRYTYWGQGTLVTVSS |
| Exemplary anti-target | EVQLVESGGGLVQPGGSLTLSCAASGSSVKFLSMAWYRQAPG |
| (DLL3) | KKRELVAGISLDGSTDYIDSVKGRFTISRDNAKNSVYLQMNS |
| (SEQ ID NO: 548) | LRAEDTAVYYCYAYRWTGRYTYWGQGTLVTVSS |
| Exemplary anti-target | EVQLVESGGGLVQPGGSLTLSCAASGSSVSFLSMAWYRQAPG |
| (DLL3) | KKRELVAGISADGSTIYIDSVKGRFTISRDNAKNSVYLQMNS |
| (SEQ ID NO: 549) | LRAEDTAVYYCYAYRWRTRYTYWGQGTLVTVSS |
| Exemplary anti-target | EVQLVESGGGLVQPGGSLTLSCAASGSSVRFLSMAWYRQAPG |
| (DLL3) | KKRELVAGISAHGSTDYIDSVKGRFTISRDNAKNSVYLQMNS |
| (SEQ ID NO: 550) | LRAEDTAVYYCYAYRWRTRYTYWGQGTLVTVSS |
| Exemplary anti-target | EVQLVESGGGLVQPGGSLTLSCAASGSSVSFLSMAWYRQAPG |
| (DLL3) | KKRELVAGISRDGSTDYIDSVKGRFTISRDNAKNSVYLQMNS |
| (SEQ ID NO: 551) | LRAEDTAVYYCYAYRWITRYTYWGQGTLVTVSS |
| Exemplary anti-target | EVQLVESGGGLVQPGGSLTLSCAASGSSVRFLSMAWYRQAPG |
| (DLL3) | KKRELVAGISRDGSTDYIDSVKGRFTISRDNAKNSVYLQMNS |
| (SEQ ID NO: 552) | LRAEDTAVYYCYAYRWITRYTYWGQGTLVTVSS |
| Exemplary anti-target | EVQLVESGGGLVQPGGSLTLSCAASGSSVVELSMAWYRQAPG |
| (DLL3) | KKRELVAGISADGSMDYIDSVKGRFTISRDNAKNSVYLQMNS |
| (SEQ ID NO: 553) | LRAEDTAVYYCYAYRWRTRYTYWGQGTLVTVSS |
| Exemplary anti-target | EVQLVESGGGLVQPGGSLTLSCAASGSSVRFLSMAWYRQAPG |
| (DLL3) | KKRELVAGISADGSTDYIDSVKGRFTISRDNAKNSVYLQMNS |
| (SEQ ID NO: 554) | LRAEDTAVYYCYAYRWTRRYTYWGQGTLVTVSS |
| Exemplary anti-target | EVQLVESGGGLVQPGGSLTLSCAASGSSVRFLSMAWYRQAPG |
| (DLL3) | KKRELVAGISADGSTDYIDSVKGRFTISRDNAKNSVYLQMNS |
| (SEQ ID NO: 555) | LRAEDTAVYYCYAYSWTTRYTYWGQGTLVTVSS |
| Exemplary anti-target | EVQLVESGGGLVQPGGSLTLSCAASGSSVRFLSMAWYRQAPG |
| (DLL3) | KKRELVAGISANGSTDYIDSVKGRFTISRDNAKNSVYLQMNS |
| (SEQ ID NO: 556) | LRAEDTAVYYCYAYRWTNRYTYWGQGTLVTVSS |
| Exemplary anti-target | EVQLVESGGGLVQPGGSLTLSCAASGSSVSRLSMAWYRQAPG |
| (DLL3) | KKRELVAGISANGSTTYIDSVKGRFTISRDNAKNSVYLQMNS |
| (SEQ ID NO: 557) | LRAEDTAVYYCYAYRWTTRYRYWGQGTLVTVSS |
| Exemplary anti-target | EVQLVESGGGLVQPGGSLTLSCAASGSSKSFLSMAWYRQAPG |
| (DLL3) | KKRELVAGISADGSTSYIDSVKGRFTISRDNAKNSVYLQMNS |
| (SEQ ID NO: 558) | LRAEDTAVYYCYAYRWTTRYTYWGQGTLVTVSS |
| Exemplary anti-target | EVQLVESGGGLVQPGGSLTLSCAASGSSVSRLSMAWYRQAPG |
| (DLL3) | KKRELVAGISADGSRDYIDSVKGRFTISRDNAKNSVYLQMNS |
| (SEQ ID NO: 559) | LRAEDTAVYYCYAYRWTTRYKYWGQGTLVTVSS |
| Exemplary anti-target | EVQLVESGGGLVQPGGSLTLSCAASGSSVKFLSMAWYRQAPG |
| (DLL3) | KKRELVAGISADGSTMYIDSVKGRFTISRDNAKNSVYLQMNS |
| (SEQ ID NO: 560) | LRAEDTAVYYCYAYRWHTRYTYWGQGTLVTVSS |
| Exemplary anti-target | EVQLVESGGGLVQPGGSLTLSCAASGSGVRFLSMAWYRQAPG |
| (DLL3) | KKRELVAGISPDGSTDYIDSVKGRFTISRDNAKNSVYLQMNS |
| (SEQ ID NO: 561) | LRAEDTAVYYCYAYRWTRRYTYWGQGTLVTVSS |
| Exemplary anti-target | EVQLVESGGGLVQPGGSLTLSCAASGSSVRFLSMAWYRQAPG |
| (DLL3) | KKRELVAGISGDGSTDYIDSVKGRFTISRDNAKNSVYLQMNS |
| (SEQ ID NO: 562) | LRAEDTAVYYCYAYRWMTRYTYWGQGTLVTVSS |
| Exemplary anti-target | EVQLVESGGGLVQPGGSLTLSCAASGSSVHFLSMAWYRQAPG |
| (DLL3) | KKRELVAGISRDGSTDYIDSVKGRFTISRDNAKNSVYLQMNS |
| (SEQ ID NO: 563) | LRAEDTAVYYCYAYRWTTRYRYWGQGTLVTVSS |
| Exemplary anti-target | EVQLVESGGGLVQPGGSLTLSCAASGSSVRFLSMAWYRQAPG |
| (DLL3) | KKRELVAGISRDGSTDYIDSVKGRFTISRDNAKNSVYLQMNS |
| (SEQ ID NO: 564) | LRAEDTAVYYCYAYRWSTRYTYWGQGTLVTVSS |
| Exemplary anti-target | EVQLVESGGGLVQPGGSLTLSCAASGSKVSFLSMAWYRQAPG |
| (DLL3) | KKRELVAGISRDGSTDYIDSVKGRFTISRDNAKNSVYLQMNS |
| (SEQ ID NO: 565) | LRAEDTAVYYCYAYRWTTRYTFWGQGTLVTVSS |
| Exemplary anti-target | EVQLVESGGGLVQPGGSLTLSCAASGSSVSFLSMAWYRQAPG |
| (DLL3) | KKRELVAGISADGSTDYIDSVKGRFTISRDNAKNSVYLQMNS |
| (SEQ ID NO: 566) | LRAEDTAVYYCYAYRWRTRYTYWGQGTLVTVSS |
| Exemplary anti-target | EVQLVESGGGLVQPGGSLTLSCAASGSKVSFLSMAWYRQAPG |
| (DLL3) | KKRELVAGISTDGSTDYIDSVKGRFTISRDNAKNSVYLQMNS |
| (SEQ ID NO: 567) | LRAEDTAVYYCYAYRWTTRRTYWGQGTLVTVSS |
| Exemplary anti-target | EVQLVESGGGLVQPGGSLTLSCAASGSRVSFLSMAWYRQAPG |
| (DLL3) | KKRELVAGISADGSTSYIDSVKGRFTISRDNAKNSVYLQMNS |
| (SEQ ID NO: 568) | LRAEDTAVYYCYAYRWATRYTYWGQGTLVTVSS |
| Exemplary anti-target | EVQLVESGGGLVQPGGSLTLSCAASGSRVSFLSMAWYRQAPG |
| (DLL3) | KKRELVAGISADGSTLYIDSVKGRFTISRDNAKNSVYLQMNS |
| (SEQ ID NO: 569) | LRAEDTAVYYCYAYRWHTRYTYWGQGTLVTVSS |
| Exemplary anti-target | EVQLVESGGGLVQPGGSLTLSCAASGSSVRFLSMAWYRQAPG |
| (DLL3) | KKRELVAGISRDGSTDYIDSVKGRFTISRDNAKNSVYLQMNS |
| (SEQ ID NO: 570) | LRAEDTAVYYCYAYRWGTRYTYWGQGTLVTVSS |
| Exemplary anti-target | EVQLVESGGGLVQPGGSLTLSCAASYSSVSRLSMAWYRQAPG |
| (DLL3) | KKRELVAGISADGSTVYIDSVKGRFTISRDNAKNSVYLQMNS |
| (SEQ ID NO: 571) | LRAEDTAVYYCYAYRWTTRNTYWGQGTLVTVSS |
| Exemplary anti-target | EVQLVESGGGLVQPGGSLTLSCAASGSSVSFLSMAWYRQAPG |
| (DLL3) | KKRELVAGISTDGSTDYIDSVKGRFTISRDNAKNSVYLQMNS |
| (SEQ ID NO: 572) | LRAEDTAVYYCYAYRWRTRYTYWGQGTLVTVSS |
| Exemplary anti-target | EVQLVESGGGLVQPGGSLTLSCAASGSSVSFLSMAWYRQAPG |
| (DLL3) | KKRELVAGISADGSTLYIDSVKGRFTISRDNAKNSVYLQMNS |
| (SEQ ID NO: 573) | LRAEDTAVYYCYAYRWTTRYAYWGQGTLVTVSS |
| Exemplary anti-target | EVQLVESGGGLVQPGGSLTLSCAASGSSVSFLSMAWYRQAPG |
| (DLL3) | KKRELVAGISADGRTDYIDSVKGRFTISRDNAKNSVYLQMNS |
| (SEQ ID NO: 574) | LRAEDTAVYYCYAYRWRTRYTYWGQGTLVTVSS |
| Exemplary anti-target | EVQLVESGGGLVQPGGSLTLSCVASGTSVSFLSMAWYRQAPG |
| (DLL3) | KKRELVAGISADGSTIYIDSVKGRFTISRDNAKNSVYLQMNS |
| (SEQ ID NO: 575) | LRAEDTAVYYCYAYRWTTRRTYWGQGTLVTVSS |
| Exemplary anti-target | EVQLVESGGGLVQPGGSLTLSCAASGSSVKFLSMAWYRQAPG |
| (DLL3) | KKRELVAGISADGSTLYIDSVKGRFTISRDNAKNSVYLQMNS |
| (SEQ ID NO: 576) | LRAEDTAVYYCYAYRWTTRRTYWGQGTLVTVSS |
| Exemplary anti-target | EVQLVESGGGLVQPGGSLTLSCAASGSSVSFLSMAWYRQAPG |
| (DLL3) | KKRELVAGISRDGSTDYIDSVKGRFTISRDNAKNSVYLQMNS |
| (SEQ ID NO: 577) | LRAEDTAVYYCYAYRWTSRYTYWGQGTLVTVSS |
| Exemplary anti-target | EVQLVESGGGLVQPGGSLTLSCAASGSRVSFLSMAWYRQAPG |
| (DLL3) | KKRELVAGISKDGSTDYIDSVKGRFTISRDNAKNSVYLQMNS |
| (SEQ ID NO: 578) | LRAEDTAVYYCYAYRWTTRVTYWGQGTLVTVSS |
| Exemplary anti-target | EVQLVESGGGLVQPGGSLTLSCAASGSSVSVLSMAWYRQAPG |
| (DLL3) | KKRELVAGISADGSTDYIGSVKGRFTISRDNAKNSVYLQMNS |
| (SEQ ID NO: 579) | LRAEDTAVYYCYAYRWTTRTTYWGQGTLVTVSS |
| Exemplary anti-target | EVQLVESGGGLVQPGGSLTLSCAASGSSVSFLSMAWYRQAPG |
| (DLL3) | KKRELVAGISVDGSTDYIDSVKGRFTISRDNAKNSVYLQMNS |
| (SEQ ID NO: 580) | LRAEDTAVYYCYAYRWRTRYTYWGQGTLVTVSS |
| Exemplary anti-target | EVQLVESGGGLVQPGGSLTLSCAASGSRVSFLSMAWYRQAPG |
| (DLL3) | KKRELVAGISADGSTGYIDSVKGRFTISRDNAKNSVYLQMNS |
| (SEQ ID NO: 581) | LRAEDTAVYYCYAYRWATRYTYWGQGTLVTVSS |
| Exemplary anti-target | EVQLVESGGGLVQPGGSLTLSCVASGSSVKFLSMAWYRQAPG |
| (DLL3) | KKRELVAGISGDGSTTYIDSVKGRFTISRDNAKNSVYLQMNS |
| (SEQ ID NO: 582) | LRAEDTAVYYCYAYRWTTRRTYWGQGTLVTVSS |
| Exemplary anti-target | EVQLVESGGGLVQPGGSLTLSCAASGSSVRFLSMAWYRQAPG |
| (DLL3) | KKRELVAGISTDGSTDYIDSVKGRFTISRDNAKNSVYLQMNS |
| (SEQ ID NO: 583) | LRAEDTAVYYCYALRWTTRYTYWGQGTLVTVSS |
| Exemplary anti-target | EVQLVESGGGLVQPGGSLTLSCAASGSSVSQLSMAWYRQAPG |
| (DLL3) | KKRELVAGISADGSTDYFDSVKGRFTISRDNAKNSVYLQMNS |
| (SEQ ID NO: 584) | LRAEDTAVYYCYAYRWTTRGTYWGQGTLVTVSS |
| Exemplary anti-target | EVQLVESGGGLVQPGGSLTLSCAASGSRVSFLSMAWYRQAPG |
| (DLL3) | KKRELVAGISADGSTSYIDSVKGRFTISRDNAKNSVYLQMNS |
| (SEQ ID NO: 585) | LRAEDTAVYYCYAYRWRTRYTYWGQGTLVTVSS |
| Exemplary anti-target | EVQLVESGGGLVQPGGSLTLSCAASKSSVSFLSMAWYRQAPG |
| (DLL3) | KKRELVAGISADGSTDYIDSVKGRFTISRDNAKNSVYLQMNS |
| (SEQ ID NO: 586) | LRAEDTAVYYCYAYRWTTRATYWGQGTLVTVSS |
| Exemplary anti-target | EVQLVESGGGLVQPGGSLTLSCAASGSSVSFLSMAWYRQAPG |
| (DLL3) | KKRELVAGISADGSTAYIDSVKGRFTISRDNAKNSVYLQMNS |
| (SEQ ID NO: 587) | LRAEDTAVYYCYAYRWTTRRTYWGQGTLVTVSS |
| Exemplary anti-target | EVQLVESGGGLVQPGGSLTLSCAASGSSVKFLSMAWYRQAPG |
| (DLL3) | KKRELVAGISADGSTVYIDSVKGRFTISRDNAKNSVYLQMNS |
| (SEQ ID NO: 588) | LRAEDTAVYYCYAYRWPTRYTYWGQGTLVTVSS |
| Exemplary anti-target | EVQLVESGGGLVQPGGSLTLSCAASGSSVRFLSMAWYRQAPG |
| (DLL3) | KKRELVAGISQDGSTDYIDSVKGRFTISRDNAKNSVYLQMNS |
| (SEQ ID NO: 589) | LRAEDTAVYYCYAYRWRTRYTYWGQGTLVTVSS |
| Exemplary anti-target | EVQLVESGGGLVQPGGSLTLSCAASGSSVRFLSMAWYRQAPG |
| (DLL3) | KKRELVAGISNDGSTDYIDSVKGRFTISRDNAKNSVYLQMNS |
| (SEQ ID NO: 590) | LRAEDTAVYYCYAYRWKTRYTYWGQGTLVTVSS |
| Exemplary anti-target | EVQLVESGGGLVQPGGSLTLSCAASGSRVSFLSMAWYRQAPG |
| (DLL3) | KKRELVAGISARGSTDYIDSVKGRFTISRDNAKNSVYLQMNS |
| (SEQ ID NO: 591) | LRAEDTAVYYCYAYRWSTRYTYWGQGTLVTVSS |
| Exemplary anti-target | EVQLVESGGGLVQPGGSLTLSCAASGSSVSFLSLAWYRQAPG |
| (DLL3) | KKRELVAGISADGSTDYIDSVKGRFTISRDNAKNSVYLQMNS |
| (SEQ ID NO: 592) | LRAEDTAVYYCYAYRWKTRRTYWGQGTLVTVSS |
| Exemplary anti-target | EVQLVESGGGLVQPGGSLTLSCAASGSSVRFLSMAWYRQAPG |
| (DLL3) | KKRELVAGISRDGSTDYIDSVKGRFTISRDNAKNSVYLQMNS |
| (SEQ ID NO: 593) | LRAEDTAVYYCYAYRWTTRRTYWGQGTLVTVSS |
| Exemplary anti-target | EVQLVESGGGLVQPGGSLTLSCAASGSKVSFLSMAWYRQAPG |
| (DLL3) | KKRELVAGISADGSTLYIDSVKGRFTISRDNAKNSVYLQMNS |
| (SEQ ID NO: 594) | LRAEDTAVYYCYAYRWTTRYRYWGQGTLVTVSS |
| Exemplary anti-target | EVQLVESGGGLVQPGGSLTLSCAASGSSVSFLSMAWYRQAPG |
| (DLL3) | KKRELVAGISADGSTNYIDSVKGRFTISRDNAKNSVYLQMNS |
| (SEQ ID NO: 595) | LRAEDTAVYYCYAYRWRTRYTYWGQGTLVTVSS |
| Exemplary anti-target | EVQLVESGGGLVQPGGSLTLSCAASGSSVRFLSMAWYRQAPG |
| (DLL3) | KKRELVAGISADGSTVYIDSVKGRFTISRDNAKNSVYLQMNS |
| (SEQ ID NO: 596) | LRAEDTAVYYCYAYRWTTRYKYWGQGTLVTVSS |
| Exemplary anti-target | EVQLVESGGGLVQPGGSLTLSCAASGSKVSFLSMAWYRQAPG |
| (DLL3) | KKRELVAGISADGSTTYIDSVKGRFTISRDNAKNSVYLQMNS |
| (SEQ ID NO: 597) | LRAEDTAVYYCYAYRWKTRYTYWGQGTLVTVSS |
| Exemplary anti-target | EVQLVESGGGLVQPGGSLTLSCAASGSSVKFLSMAWYRQAPG |
| (DLL3) | KKRELVAGISADGSTDYIGSVKGRFTISRDNAKNSVYLQMNS |
| (SEQ ID NO: 598) | LRAEDTAVYYCYAYRWTTRVTYWGQGTLVTVSS |
| Exemplary anti-target | EVQLVESGGGLVQPGGSLTLSCAASGSSVKFLSMAWYRQAPG |
| (DLL3) | KKRELVAGISRDGSTDYIDSVKGRFTISRDNAKNSVYLQMNS |
| (SEQ ID NO: 599) | LRAEDTAVYYCYAYRWTTRFTYWGQGTLVTVSS |
| Exemplary anti-target | EVQLVESGGGLVQPGGSLTLSCAASGSRVSFLSMAWYRQAPG |
| (DLL3) | KKRELVAGISADGSTTYIDSVKGRFTISRDNAKNSVYLQMNS |
| (SEQ ID NO: 600) | LRAEDTAVYYCYAYRWTTRFTYWGQGTLVTVSS |
| Exemplary anti-target | EVQLVESGGGLVQPGGSLTLSCAASGSSVLFLSMAWYRQAPG |
| (DLL3) | KKRELVAGVSSDGSTDYIDSVKGRFTISRDNAKNSVYLQMNS |
| (SEQ ID NO: 601) | LRAEDTAVYYCYAYRWRTRYTYWGQGTLVTVSS |
| Exemplary anti-target | EVQLVESGGGLVQPGGSLTLSCAASGSRVSFLSMAWYRQAPG |
| (DLL3) | KKRELVAGISADGHTDYIDSVKGRFTISRDNAKNSVYLQMNS |
| (SEQ ID NO: 602) | LRAEDTAVYYCYAYRWTTRYTHWGQGTLVTVSS |
| Exemplary anti-target | EVQLVESGGGLVQPGGSLTLSCAASGSRVSFLSMAWYRQAPG |
| (DLL3) | KKRELVAGISADGSTDYFDSVKGRFTISRDNAKNSVYLQMNS |
| (SEQ ID NO: 603) | LRAEDTAVYYCYAYRWTRRYTYWGQGTLVTVSS |
| Exemplary anti-target | EVQLVESGGGLVQPGGSLTLSCAASGSSVGFLSMAWYRQAPG |
| (DLL3) | KKRELVAGISADGSTVYIDSVKGRFTISRDNAKNSVYLQMNS |
| (SEQ ID NO: 604) | LRAEDTAVYYCYAYRWTTRYTYWGQGTLVTVSS |
| Exemplary anti-target | EVQLVESGGGLVQPGGSLTLSCAASGSSVSFMSMAWYRQAPG |
| (DLL3) | KKRELVAGISADGSTDYIASVKGRFTISRDNAKNSVYLQMNS |
| (SEQ ID NO: 605) | LRAEDTAVYYCYAYRWTTRSTYWGQGTLVTVSS |
| Exemplary anti-target | EVQLVESGGGLVQPGGSLTLSCAASGSSVSFLSMAWYRQAPG |
| (DLL3) | KKRELVAGISADGSTDYISSVKGRFTISRDNAKNSVYLQMNS |
| (SEQ ID NO: 606) | LRAEDTAVYYCYAYSWTTRYTYWGQGTLVTVSS |
| Exemplary anti-target | EVQLVESGGGLVQPGGSLTLSCAASGSSVTFLSMAWYRQAPG |
| (DLL3) | KKRELVAGISADGSTVYIDSVKGRFTISRDNAKNSVYLQMNS |
| (SEQ ID NO: 607) | LRAEDTAVYYCYAYRWTTRGTYWGQGTLVTVSS |
| Exemplary anti-target | EVQLVESGGGLVQPGGSLTLSCAASGSSVRFLSMAWYRQAPG |
| (DLL3) | KKRELVAGISADGSTVYIDSVKGRFTISRDNAKNSVYLQMNS |
| (SEQ ID NO: 608) | LRAEDTAVYYCYAYRWKTRYTYWGQGTLVTVSS |
| Exemplary anti-target | EVQLVESGGGLVQPGGSLTLSCAASGSKVSFLSMAWYRQAPG |
| (DLL3) | KKRELVAGISADGSTTYIDSVKGRFTISRDNAKNSVYLQMNS |
| (SEQ ID NO: 609) | LRAEDTAVYYCYAYRWTTRFTYWGQGTLVTVSS |
| Exemplary anti-target | EVQLVESGGGLVQPGGSLTLSCAASGSKVSFMSMAWYRQAPG |
| (DLL3) | KKRELVAGISVDGSTDYIDSVKGRFTISRDNAKNSVYLQMNS |
| (SEQ ID NO: 610) | LRAEDTAVYYCYAYRWRTRYTYWGQGTLVTVSS |
| Exemplary anti-target | EVQLVESGGGLVQPGGSLTLSCAASGSSVSNLSMAWYRQAPG |
| (DLL3) | KKRELVAGISADGSTAYIDSVKGRFTISRDNAKNSVYLQMNS |
| (SEQ ID NO: 611) | LRAEDTAVYYCYAYRWTTRRTYWGQGTLVTVSS |
| Exemplary anti-target | EVQLVESGGGLVQPGGSLTLSCAASNSSVSKLSMAWYRQAPG |
| (DLL3) | KKRELVAGISADGSTAYIDSVKGRFTISRDNAKNSVYLQMNS |
| (SEQ ID NO: 612) | LRAEDTAVYYCYAYRWSTRYTYWGQGTLVTVSS |
| Exemplary anti-target | EVQLVESGGGLVQPGGSLTLSCAASGSKVSFLSMAWYRQAPG |
| (DLL3) | KKRELVAGISADGSKDYIDSVKGRFTISRDNAKNSVYLQMNS |
| (SEQ ID NO: 613) | LRAEDTAVYYCYAYRWTTRLTYWGQGTLVTVSS |
| Exemplary anti-target | EVQLVESGGGLVQPGGSLTLSCVASGSQVSFLSMAWYRQAPG |
| (DLL3) | KKRELVAGISADGSTDYFDSVKGRFTISRDNAKNSVYLQMNS |
| (SEQ ID NO: 614) | LRAEDTAVYYCYAYRWTRRYTYWGQGTLVTVSS |
| Exemplary anti-target | EVQLVESGGGLVQPGGSLTLSCAASGSKVSFMSMAWYRQAPG |
| (DLL3) | KKRELVAGISADGSTDYIDSVKGRFTISRDNAKNSVYLQMNS |
| (SEQ ID NO: 615) | LRAEDTAVYYCYAYRWTTRLTYWGQGTLVTVSS |
| Exemplary anti-target | EVQLVESGGGLVQPGGSLTLSCAASGSRVSFLSMAWYRQAPG |
| (DLL3) | KKRELVAGISADGSTVYIDSVKGRFTISRDNAKNSVYLQMNS |
| (SEQ ID NO: 616) | LRAEDTAVYYCYAYRWTRRYTYWGQGTLVTVSS |
| Exemplary anti-target | EVQLVESGGGLVQPGGSLTLSCAASGSSVRFLSMAWYRQAPG |
| (DLL3) | KKRELVAGISADGSTVYIDSVKGRFTISRDNAKNSVYLQMNS |
| (SEQ ID NO: 617) | LRAEDTAVYYCYAYRWTTRRTYWGQGTLVTVSS |
| Exemplary anti-target | EVQLVESGGGLVQPGGSLTLSCAASGSKVSFLSMAWYRQAPG |
| (DLL3) | KKRELVAGISARGSTDYIDSVKGRFTISRDNAKNSVYLQMNS |
| (SEQ ID NO: 618) | LRAEDTAVYYCYAYQWTTRYTYWGQGTLVTVSS |
| Exemplary anti-target | EVQLVESGGGLVQPGGSLTLSCAASGSSVRFLSMAWYRQAPG |
| (DLL3) | KKRELVAGISATGSTDYIDSVKGRFTISRDNAKNSVYLQMNS |
| (SEQ ID NO: 619) | LRAEDTAVYYCYAYRWTRRYTYWGQGTLVTVSS |
| Exemplary anti-target | EVQLVESGGGLVQPGGSLTLSCAASGSSVSFLSIAWYRQAPG |
| (DLL3) | KKRELVAGISKDGSTDYIDSVKGRFTISRDNAKNSVYLQMNS |
| (SEQ ID NO: 620) | LRAEDTAVYYCYAYRWTTRMTYWGQGTLVTVSS |
| Exemplary anti-target | EVQLVESGGGLVQPGGSLTLSCAASGSSSSFLSMAWYRQAPG |
| (DLL3) | KKRELVAGISADGSTVYIDSVKGRFTISRDNAKNSVYLQMNS |
| (SEQ ID NO: 621) | LRAEDTAVYYCYAYRWTTRRTYWGQGTLVTVSS |
| Exemplary anti-target | EVQLVESGGGLVQPGGSLTLSCAASGSSVKFLSMAWYRQAPG |
| (DLL3) | KKRELVAGISPDGSTDYIDSVKGRFTISRDNAKNSVYLQMNS |
| (SEQ ID NO: 622) | LRAEDTAVYYCYAYRWTTRYRYWGQGTLVTVSS |
| Exemplary anti-target | EVQLVESGGGLVQPGGSLTLSCAASGSSVNFLSMAWYRQAPG |
| (DLL3) | KKRELVAGISADGSTHYIDSVKGRFTISRDNAKNSVYLQMNS |
| (SEQ ID NO: 623) | LRAEDTAVYYCYAYRWLTRYTYWGQGTLVTVSS |
| Exemplary anti-target | EVQLVESGGGLVQPGGSLTLSCAASGSSVKFLSMAWYRQAPG |
| (DLL3) | KKRELVAGISADGSTDYILSVKGRFTISRDNAKNSVYLQMNS |
| (SEQ ID NO: 624) | LRAEDTAVYYCYAYEWTTRYTYWGQGTLVTVSS |
| Exemplary anti-target | EVQLVESGGGLVQPGGSLTLSCAASGSSVSFLSMAWYRQAPG |
| (DLL3) | KKRELVAGISADGSTDYIHSVKGRFTISRDNAKNSVYLQMNS |
| (SEQ ID NO: 625) | LRAEDTAVYYCYAYRWRTRYTYWGQGTLVTVSS |
| Exemplary anti-target | EVQLVESGGGLVQPGGSLTLSCAASGSSVRFLSMAWYRQAPG |
| (DLL3) | KKRELVAGISVDGSTDYIDSVKGRFTISRDNAKNSVYLQMNS |
| (SEQ ID NO: 626) | LRAEDTAVYYCYAYRWRTRYTYWGQGTLVTVSS |
| Exemplary anti-target | EVQLVESGGGLVQPGGSLTLSCAASGSSVSFLSVAWYRQAPG |
| (DLL3) | KKRELVAGISRDGSTDYIDSVKGRFTISRDNAKNSVYLQMNS |
| (SEQ ID NO: 627) | LRAEDTAVYYCYAYRWRTRYTYWGQGTLVTVSS |
| Exemplary anti-target | EVQLVESGGGLVQPGGSLTLSCAASGSQVSFLSMAWYRQAPG |
| (DLL3) | KKRELVAGISADGSTVYIDSVKGRFTISRDNAKNSVYLQMNS |
| (SEQ ID NO: 628) | LRAEDTAVYYCYAYRWSTRYTYWGQGTLVTVSS |
| Exemplary anti-target | EVQLVESGGGLVQPGGSLTLSCAASGTSVSFLSMAWYRQAPG |
| (DLL3) | KKRELVAGISADGSTDYIRSVKGRFTISRDNAKNSVYLQMNS |
| (SEQ ID NO: 629) | LRAEDTAVYYCYAYRWTTRLTYWGQGTLVTVSS |
| Exemplary anti-target | EVQLVESGGGLVQPGGSLTLSCAASGSRVSFLSMAWYRQAPG |
| (DLL3) | KKRELVAGISADGSTMYIDSVKGRFTISRDNAKNSVYLQMNS |
| (SEQ ID NO: 630) | LRAEDTAVYYCYAYRWTTRLTYWGQGTLVTVSS |
| Exemplary anti-target | EVQLVESGGGLVQPGGSLTLSCAASGSSVKFLSMAWYRQAPG |
| (DLL3) | KKRELVAGISTDGSTDYIDSVKGRFTISRDNAKNSVYLQMNS |
| (SEQ ID NO: 631) | LRAEDTAVYYCYAYKWTTRYTYWGQGTLVTVSS |
| Exemplary anti-target | EVQLVESGGGLVQPGGSLTLSCAASGSSVSFLSSAWYRQAPG |
| (DLL3) | KKRELVAGISADGSTLYIDSVKGRFTISRDNAKNSVYLQMNS |
| (SEQ ID NO: 632) | LRAEDTAVYYCYAYRWTTRSTYWGQGTLVTVSS |
| Exemplary anti-target | EVQLVESGGGLVQPGGSLTLSCAASGSSVKFLSMAWYRQAPG |
| (DLL3) | KKRELVAGISADGSTDYIDSVKGRFTISRDNAKNSVYLQMNS |
| (SEQ ID NO: 633) | LRAEDTAVYYCYAYRWRTRYTYWGQGTLVTVSS |
| Exemplary anti-target | EVQLVESGGGLVQPGGSLTLSCAASGSSVSFLSMAWYRQAPG |
| (DLL3) | KKRELVAGISATGSTDYIDSVKGRFTISRDNAKNSVYLQMNS |
| (SEQ ID NO: 634) | LRAEDTAVYYCYAYRWSTRYTYWGQGTLVTVSS |
| Exemplary anti-target | EVQLVESGGGLVQPGGSLTLSCAASGSTVSFLSMAWYRQAPG |
| (DLL3) | KKRELVAGISHDGSTDYIDSVKGRFTISRDNAKNSVYLQMNS |
| (SEQ ID NO: 635) | LRAEDTAVYYCYAYRWTTRYTYWGQGTLVTVSS |
| Exemplary anti-target | EVQLVESGGGLVQPGGSLTLSCAASGSSVQFLSMAWYRQAPG |
| (DLL3) | KKRELVAGISYDGSTDYIDSVKGRFTISRDNAKNSVYLQMNS |
| (SEQ ID NO: 636) | LRAEDTAVYYCYAYRWRTRYTYWGQGTLVTVSS |
| Exemplary anti-target | EVQLVESGGGLVQPGGSLTLSCAASRSSVSFLSMAWYRQAPG |
| (DLL3) | KKRELVAGISTDGSTDYIDSVKGRFTISRDNAKNSVYLQMNS |
| (SEQ ID NO: 637) | LRAEDTAVYYCYAYRWLTRYTYWGQGTLVTVSS |
| Exemplary anti-target | EVQLVESGGGLVQPGGSLTLSCAASGSKVSFLSMAWYRQAPG |
| (DLL3) | KKRELVAGISADGSTAYIDSVKGRFTISRDNAKNSVYLQMNS |
| (SEQ ID NO: 638) | LRAEDTAVYYCYAYRWRTRYTYWGQGTLVTVSS |
| Exemplary anti-target | EVQLVESGGGLVQPGGSLTLSCAASGSRVSFLSMAWYRQAPG |
| (DLL3) | KKRELVAGISADGSTDYIESVKGRFTISRDNAKNSVYLQMNS |
| (SEQ ID NO: 639) | LRAEDTAVYYCYAYRWTTRYTYWGQGTLVTVSS |
| Exemplary anti-target | EVQLVESGGGLVQPGGSLTLSCAASGSSVSFLSMAWYRQAPG |
| (DLL3) | KKRELVAGISIDGSTDYIKSVKGRFTISRDNAKNSVYLQMNS |
| (SEQ ID NO: 640) | LRAEDTAVYYCYAYRWTTRYRYWGQGTLVTVSS |
| Exemplary anti-target | EVQLVESGGGLVQPGGSLTLSCAASGSKVSFLSMAWYRQAPG |
| (DLL3) | KKRELVAGISADGSKDYIDSVKGRFTISRDNAKNSVYLQMNS |
| (SEQ ID NO: 641) | LRAEDTAVYYCYAYRWTTRYTYWGQGTLVTVSS |
| Exemplary anti-target | EVQLVESGGGLVQPGGSLTLSCAASGSSVRFLSMAWYRQAPG |
| (DLL3) | KKRELVAGISADGSTVYIDSVKGRFTISRDNAKNSVYLQMNS |
| (SEQ ID NO: 642) | LRAEDTAVYYCYAYRWPTRYTYWGQGTLVTVSS |
| Exemplary anti-target | EVQLVESGGGLVQPGGSLTLSCAASGSSVKFLSMAWYRQAPG |
| (DLL3) | KKRELVAGISRDGSTDYIDSVKGRFTISRDNAKNSVYLQMNS |
| (SEQ ID NO: 643) | LRAEDTAVYYCYAYRWTTRHTYWGQGTLVTVSS |
| Exemplary anti-target | EVQLVESGGGLVQPGGSLTLSCAASGSSVKFLSMAWYRQAPG |
| (DLL3) | KKRELVAGISADGSTDYIHSVKGRFTISRDNAKNSVYLQMNS |
| (SEQ ID NO: 644) | LRAEDTAVYYCYAYRWTRRYTYWGQGTLVTVSS |
| Exemplary anti-target | EVQLVESGGGLVQPGGSLTLSCAASGSSVSILSMAWYRQAPG |
| (DLL3) | KKRELVAGISADGSTIYIDSVKGRFTISRDNAKNSVYLQMNS |
| (SEQ ID NO: 645) | LRAEDTAVYYCYAYRWHTRYTYWGQGTLVTVSS |
| Exemplary anti-target | EVQLVESGGGLVQPGGSLTLSCAASGSSVSFLSVAWYRQAPG |
| (DLL3) | KKRELVAGISANGSTDYIDSVKGRFTISRDNAKNSVYLQMNS |
| (SEQ ID NO: 646) | LRAEDTAVYYCYAYRWINRYTYWGQGTLVTVSS |
| Exemplary anti-target | EVQLVESGGGLVQPGGSLTLSCAASGSSVRFLSMAWYRQAPG |
| (DLL3) | KKRELVAGISTDGSTDYIDSVKGRFTISRDNAKNSVYLQMNS |
| (SEQ ID NO: 647) | LRAEDTAVYYCYAYRWTTRYRYWGQGTLVTVSS |
| Exemplary anti-target | EVQLVESGGGLVQPGGSLTLSCAASGSRVSFLSMAWYRQAPG |
| (DLL3) | KKRELVAGISYDGSTDYIDSVKGRFTISRDNAKNSVYLQMNS |
| (SEQ ID NO: 648) | LRAEDTAVYYCYAYRWTTRRTYWGQGTLVTVSS |
| Exemplary anti-target | EVQLVESGGGLVQPGGSLTLSCAASGHSVSFLSMAWYRQAPG |
| (DLL3) | KKRELVAGISADGSTDYIASVKGRFTISRDNAKNSVYLQMNS |
| (SEQ ID NO: 649) | LRAEDTAVYYCYAYRWSTRYTYWGQGTLVTVSS |
| Exemplary anti-target | EVQLVESGGGLVQPGGSLTLSCAASGSSVRFLSMAWYRQAPG |
| (DLL3) | KKRELVAGISADGSTDYIGSVKGRFTISRDNAKNSVYLQMNS |
| (SEQ ID NO: 650) | LRAEDTAVYYCYAYRWSTRYTYWGQGTLVTVSS |
| Exemplary anti-target | EVQLVESGGGLVQPGGSLTLSCAASGSSVSFLSMAWYRQAPG |
| (DLL3) | KKRELVAGISANGSTDYYDSVKGRFTISRDNAKNSVYLQMNS |
| (SEQ ID NO: 651) | LRAEDTAVYYCYAYRWRTRYTYWGQGTLVTVSS |
| Exemplary anti-target | EVQLVESGGGLVQPGGSLTLSCAASGSRVSFLSMAWYRQAPG |
| (DLL3) | KKRELVAGISADGSTSYIDSVKGRFTISRDNAKNSVYLQMNS |
| (SEQ ID NO: 652) | LRAEDTAVYYCYAYRWTTRYTYWGQGTLVTVSS |
| Exemplary anti-target | EVQLVESGGGLVQPGGSLTLSCAASGSSVKFLSMAWYRQAPG |
| (DLL3) | KKRELVAGVSADGSTDYIDSVKGRFTISRDNAKNSVYLQMNS |
| (SEQ ID NO: 653) | LRAEDTAVYYCYAYEWTTRYTYWGQGTLVTVSS |
| Exemplary anti-target | EVQLVESGGGLVQPGGSLTLSCAASGSSVSRLSMAWYRQAPG |
| (DLL3) | KKRELVAGISARGSTDYIDSVKGRFTISRDNAKNSVYLQMNS |
| (SEQ ID NO: 654) | LRAEDTAVYYCYAYRWTTRSTYWGQGTLVTVSS |
| Exemplary anti-target | EVQLVESGGGLVQPGGSLTLSCAASGRSVSFLSMAWYRQAPG |
| (DLL3) | KKRELVAGISADGSTIYIDSVKGRFTISRDNAKNSVYLQMNS |
| (SEQ ID NO: 655) | LRAEDTAVYYCYAYRWTTRYTYWGQGTLVTVSS |
| Exemplary anti-target | EVQLVESGGGLVQPGGSLTLSCAASGRSVSFLSMAWYRQAPG |
| (DLL3) | KKRELVAGISANGSTDYIDSVKGRFTISRDNAKNSVYLQMNS |
| (SEQ ID NO: 656) | LRAEDTAVYYCYAYRWSTRYTYWGQGTLVTVSS |
| Exemplary anti-target | EVQLVESGGGLVQPGGSLTLSCAASGSSVKFLSMAWYRQAPG |
| (DLL3) | KKRELVAGISADGSTDYVDSVKGRFTISRDNAKNSVYLQMNS |
| (SEQ ID NO: 657) | LRAEDTAVYYCYAYRWSTRYTYWGQGTLVTVSS |
| Exemplary anti-target | EVQLVESGGGLVQPGGSLTLSCAASGSSVSKLSMAWYRQAPG |
| (DLL3) | KKRELVAGISADGSTDYRDSVKGRFTISRDNAKNSVYLQMNS |
| (SEQ ID NO: 658) | LRAEDTAVYYCYAYRWTYRYTYWGQGTLVTVSS |
| Exemplary anti-target | EVQLVESGGGLVQPGGSLTLSCAASGSSVSRLSMAWYRQAPG |
| (DLL3) | KKRELVAGISVDGSTDYIDSVKGRFTISRDNAKNSVYLQMNS |
| (SEQ ID NO: 659) | LRAEDTAVYYCYAYRWTTRLTYWGQGTLVTVSS |
| Exemplary anti-target | EVQLVESGGGLVQPGGSLTLSCAASGSSVKFLSLAWYRQAPG |
| (DLL3) | KKRELVAGISADGSTDYILSVKGRFTISRDNAKNSVYLQMNS |
| (SEQ ID NO: 660) | LRAEDTAVYYCYAYEWTTRYTYWGQGTLVTVSS |
| Exemplary anti-target | EVQLVESGGGLVQPGGSLTLSCAASGSSVSRLSLAWYRQAPG |
| (DLL3) | KKRELVAGISVDGSTDYIDSVKGRFTISRDNAKNSVYLQMNS |
| (SEQ ID NO: 661) | LRAEDTAVYYCYAYRWTTRLTYWGQGTLVTVSS |
| Exemplary anti-target | EVQLVESGGGLVQPGGSLTLSCVASGTSSSINAMGWYRRAPG |
| (DLL3) | KQRELVAGISSDGSKVFNESVKGRFTISRDNAKNSVYLQMNS |
| (SEQ ID NO: 662) | LRAEDTAVYYCYYFRPAAGSPMRYWGQGTLVTVSS |
| Exemplary anti-target | EVQLVESGGGLVQPGGSLTLSCVASGTTSSINAIGWYRRAPG |
| (DLL3) | KQRELVAGISSDGSEVYTDSVKGRFTISRDNAKNSVYLQMNS |
| (SEQ ID NO: 663) | LRAEDTAVYYCYYFRTVDGSPLRYWGQGTLVTVSS |
| Exemplary anti-target | EVQLVESGGGLVQPGGSLTLSCVASGSTSSINAMAWYRRAPG |
| (DLL3) | KQRELVAGISSDDSNVYYESVKGRFTISRDNAKNSVYLQMNS |
| (SEQ ID NO: 664) | LRAEDTAVYYCYYFRTVSGSSKRYWGQGTLVTVSS |
| Exemplary anti-target | EVQLVESGGGLVQPGGSLTLSCVASGQTYRVNAFGWYRRAPG |
| (DLL3) | KQRELVAGISSDGSKVYADSVKGRFTISRDNAKNSVYLQMNS |
| (SEQ ID NO: 665) | LRAEDTAVYYCYYFSAGSGTEMSYWGQGTLVTVSS |
| Exemplary anti-target | EVQLVESGGGLVQPGGSLTLSCVASGSTSSINAMAWYRRAPG |
| (DLL3) | KQRELVAGISSDESTLYVDSVKGRFTISRDNAKNSVYLQMNS |
| (SEQ ID NO: 666) | LRAEDTAVYYCYYFGSLSGSSTTYWGQGTLVTVSS |
| Exemplary anti-target | EVQLVESGGGLVQPGGSLTLSCVASGSASLTNATGWYRRAPG |
| (DLL3) | KQRELVAGISSDDSKVYSDSVKGRFTISRDNAKNSVYLQMNS |
| (SEQ ID NO: 667) | LRAEDTAVYYCYYFGSVSGSWTRYWGQGTLVTVSS |
| Exemplary anti-target | EVQLVESGGGLVQPGGSLTLSCVASGYPSLNNAMGWYRRAPG |
| (DLL3) | KQRELVAGISSDGSQVYGASVKGRFTISRDNAKNSVYLQMNS |
| (SEQ ID NO: 668) | LRAEDTAVYYCYYFRLVSGSSMSYWGQGTLVTVSS |
| Exemplary anti-target | EVQLVESGGGLVQPGGSLTLSCVASGSSSTINAIGWYRRAPG |
| (DLL3) | KQRELVAGISSDGSKVYADSVKGRFTISRDNAKNSVYLQMNS |
| (SEQ ID NO: 669) | LRAEDTAVYYCYYFRTGSGTSKSYWGQGTLVTVSS |
| Exemplary anti-target | EVQLVESGGGLVQPGGSLTLSCVASGSTSYINAMGWYRRAPG |
| (DLL3) | KQRELVAGISSDGSNMYADSVKGRFTISRDNAKNSVYLQMNS |
| (SEQ ID NO: 670) | LRAEDTAVYYCYYFSNMSGTTRRYWGQGTLVTVSS |
| Exemplary anti-target | EVQLVESGGGLVQPGGSLTLSCVASGSTSSVNALGWYRRAPG |
| (DLL3) | KQRELVAGISSDGSKVYTDSVKGRFTISRDNAKNSVYLQMNS |
| (SEQ ID NO: 671) | LRAEDTAVYYCYYFRTVPGSAMGYWGQGTLVTVSS |
| Exemplary anti-target | EVQLVESGGGLVQPGGSLTLSCVASGSTSLSNAVGWYRRAPG |
| (DLL3) | KQRELVAGISSDGSKVSAESVKGRFTISRDNAKNSVYLQMNS |
| (SEQ ID NO: 672) | LRAEDTAVYYCYYFRAESGSSMGYWGQGTLVTVSS |
| Exemplary anti-target | EVQLVESGGGLVQPGGSLTLSCVASGSTSSTNAIGWYRRAPG |
| (DLL3) | KQRELVAGISSDGSKVYDDSVKGRFTISRDNAKNSVYLQMNS |
| (SEQ ID NO: 673) | LRAEDTAVYYCYYFRTLYGSSRSYWGQGTLVTVSS |
| Exemplary anti-target | EVQLVESGGGLVQPGGSLTLSCVASGLTSTINAMGWYRRAPG |
| (DLL3) | KQRELVAGISSDGSKVYDDSVKGRFTISRDNAKNSVYLQMNS |
| (SEQ ID NO: 674) | LRAEDTAVYYCYYFSPFSGSDTGYWGQGTLVTVSS |
| Exemplary anti-target | EVQLVESGGGLVQPGGSLTLSCVASGVSPSKNAIGWYRRAPG |
| (DLL3) | KQRELVAGISSDGSAVYVGSVKGRFTISRDNAKNSVYLQMNS |
| (SEQ ID NO: 675) | LRAEDTAVYYCYYFSTFSGSSISYWGQGTLVTVSS |
| Exemplary anti-target | EVQLVESGGGLVQPGGSLTLSCVASGSTSSINAVGWYRRAPG |
| (DLL3) | KQRELVAGISSDGSYVYSESVKGRFTISRDNAKNSVYLQMNS |
| (SEQ ID NO: 676) | LRAEDTAVYYCYYFRTLAGSEMRYWGQGTLVTVSS |
| Exemplary anti-target | EVQLVESGGGLVQPGGSLTLSCVASGSTTMNNAMAWYRRAPG |
| (DLL3) | KQRELVAGISSDSSHVYADSVKGRFTISRDNAKNSVYLQMNS |
| (SEQ ID NO: 677) | LRAEDTAVYYCYYFRTVSGSGVRYWGQGTLVTVSS |
| Exemplary anti-target | EVQLVESGGGLVQPGGSLTLSCVASGSTSKINAIGWYRRAPG |
| (DLL3) | KQRELVAGISSDSSIVYTDSVKGRFTISRDNAKNSVYLQMNS |
| (SEQ ID NO: 678) | LRAEDTAVYYCYYFRPGAGHSNSYWGQGTLVTVSS |
| Exemplary anti-target | EVQLVESGGGLVQPGGSLTLSCVASGQTTALNAMGWYRRAPG |
| (DLL3) | KQRELVAGISSDGSEVNTDSVKGRFTISRDNAKNSVYLQMNS |
| (SEQ ID NO: 679) | LRAEDTAVYYCYYFRRASGTAMSYWGQGTLVTVSS |
| Exemplary anti-target | EVQLVESGGGLVQPGGSLTLSCVASGATSSINAIGWYRRAPG |
| (DLL3) | KQRELVAGISSDGSKLSSDSVKGRFTISRDNAKNSVYLQMNS |
| (SEQ ID NO: 680) | LRAEDTAVYYCYYFTSASGTDLSYWGQGTLVTVSS |
| Exemplary anti-target | EVQLVESGGGLVQPGGSLTLSCVASGSTSTINAMGWYRRAPG |
| (DLL3) | KQRELVAGISSDNSKVYADSVKGRFTISRDNAKNSVYLQMNS |
| (SEQ ID NO: 681) | LRAEDTAVYYCYYFRSANGSSKRYWGQGTLVTVSS |
| Exemplary anti-target | EVQLVESGGGLVQPGGSLTLSCVASGSTSSINAMGWYRRAPG |
| (DLL3) | KQRELVAGISSDGSRVYFDSVKGRFTISRDNAKNSVYLQMNS |
| (SEQ ID NO: 682) | LRAEDTAVYYCYYFKTIAGAGMRYWGQGTLVTVSS |
| Exemplary anti-target | EVQLVESGGGLVQPGGSLTLSCVASGSTSLVNAMGWYRRAPG |
| (DLL3) | KQRELVAGISSDGSLVYAESVKGRFTISRDNAKNSVYLQMNS |
| (SEQ ID NO: 683) | LRAEDTAVYYCYYFRYGSGSSLSYWGQGTLVTVSS |
| Exemplary anti-target | EVQLVESGGGLVQPGGSLTLSCVASGSTSLNNAIGWYRRAPG |
| (DLL3) | KQRELVAGISSDGSVVYVDSVKGRFTISRDNAKNSVYLQMNS |
| (SEQ ID NO: 684) | LRAEDTAVYYCYYFRTVPGASMKYWGQGTLVTVSS |
| Exemplary anti-target | EVQLVESGGGLVQPGGSLTLSCVASGSTSPVNAMAWYRRAPG |
| (DLL3) | KQRELVAGISSDGSKVYVDSVKGRFTISRDNAKNSVYLQMNS |
| (SEQ ID NO: 685) | LRAEDTAVYYCYYFRTVDGSAISYWGQGTLVTVSS |
| Exemplary anti-target | EVQLVESGGGLVQPGGSLTLSCVASGTTSSMNAIGWYRRAPG |
| (DLL3) | KQRELVAGISSDGSKLYDESVKGRFTISRDNAKNSVYLQMNS |
| (SEQ ID NO: 686) | LRAEDTAVYYCYYFRTVKGSGGSYWGQGTLVTVSS |
| Exemplary anti-target | EVQLVESGGGLVQPGGSLTLSCVASGETSSINAMAWYRRAPG |
| (DLL3) | KQRELVAGISSDYSKLYADSVKGRFTISRDNAKNSVYLQMNS |
| (SEQ ID NO: 687) | LRAEDTAVYYCYYFRTVSGSSRGYWGQGTLVTVSS |
| Exemplary anti-target | EVQLVESGGGLVQPGGSLTLSCVASGSTSTINAIGWYRRAPG |
| (DLL3) | KQRELVAGISSDSSKVYTESVKGRFTISRDNAKNSVYLQMNS |
| (SEQ ID NO: 688) | LRAEDTAVYYCYYFRPGPGSQMAYWGQGTLVTVSS |
| Exemplary anti-target | EVQLVESGGGLVQPGGSLTLSCVASGSTYSMNAMGWYRRAPG |
| (DLL3) | KQRELVAGISSDGSQVYVDSVKGRFTISRDNAKNSVYLQMNS |
| (SEQ ID NO: 689) | LRAEDTAVYYCYYFRTVAGSASGYWGQGTLVTVSS |
| Exemplary anti-target | EVQLVESGGGLVQPGGSLTLSCVASGSPSSINAYGWYRRAPG |
| (DLL3) | KQRELVAGISSDGSKVYSDSVKGRFTISRDNAKNSVYLQMNS |
| (SEQ ID NO: 690) | LRAEDTAVYYCYYFRTVSGSSYSYWGQGTLVTVSS |
| Exemplary anti-target | EVQLVESGGGLVQPGGSLTLSCVASGSTSTINAIGWYRRAPG |
| (DLL3) | KQRELVAGISSDGSKVYVDSVKGRFTISRDNAKNSVYLQMNS |
| (SEQ ID NO: 691) | LRAEDTAVYYCYYFINLKGSSMAYWGQGTLVTVSS |
| Exemplary anti-target | EVQLVESGGGLVQPGGSLTLSCVASGSTSSINAIGWYRRAPG |
| (DLL3) | KQRELVAGISSDGSKVYADSVKGRFTISRDNAKNSVYLQMNS |
| (SEQ ID NO: 692) | LRAEDTAVYYCYYFRMVTGSYGGYWGQGTLVTVSS |
| Exemplary anti-target | EVQLVESGGGLVQPGGSLTLSCVASGSISSINAMGWYRRAPG |
| (DLL3) | KQRELVAGISSDGSSVYADSVKGRFTISRDNAKNSVYLQMNS |
| (SEQ ID NO: 693) | LRAEDTAVYYCYYFKSSYGLPMRYWGQGTLVTVSS |
| Exemplary anti-target | EVQLVESGGGLVQPGGSLTLSCVASGSTQVNNAMAWYRRAPG |
| (DLL3) | KQRELVAGISSDGSQVYYGSVKGRFTISRDNAKNSVYLQMNS |
| (SEQ ID NO: 694) | LRAEDTAVYYCYYFKTVSGQSLRYWGQGTLVTVSS |
| Exemplary anti-target | EVQLVESGGGLVQPGGSLTLSCVASGSTASFNAMAWYRRAPG |
| (DLL3) | KQRELVAGISSDGSKVYTDSVKGRFTISRDNAKNSVYLQMNS |
| (SEQ ID NO: 695) | LRAEDTAVYYCYYFRTVTGRAARYWGQGTLVTVSS |
| Exemplary anti-target | EVQLVESGGGLVQPGGSLTLSCVASGSPLSINAIGWYRRAPG |
| (DLL3) | KQRELVAGISSDGSKVSADSVKGRFTISRDNAKNSVYLQMNS |
| (SEQ ID NO: 696) | LRAEDTAVYYCYYFGPAIGASRTYWGQGTLVTVSS |
| Exemplary anti-target | EVQLVESGGGLVQPGGSLTLSCVASGSTTFINAIGWYRRAPG |
| (DLL3) | KQRELVAGISSDGSKVYEDSVKGRFTISRDNAKNSVYLQMNS |
| (SEQ ID NO: 697) | LRAEDTAVYYCYYFRTVSGAPKSYWGQGTLVTVSS |
| Exemplary anti-target | EVQLVESGGGLVQPGGSLTLSCVASGSTSSINAIGWYRRAPG |
| (DLL3) | KQRELVAGISSDRSKVYADSVKGRFTISRDNAKNSVYLQMNS |
| (SEQ ID NO: 698) | LRAEDTAVYYCYYFHTVSGSSMSYWGQGTLVTVSS |
| Exemplary anti-target | EVQLVESGGGLVQPGGSLTLSCVASGETDTINAVGWYRRAPG |
| (DLL3) | KQRELVAGISSDGSKVYAESVKGRFTISRDNAKNSVYLQMNS |
| (SEQ ID NO: 699) | LRAEDTAVYYCYYFRRLEGYSNRYWGQGTLVTVSS |
| Exemplary anti-target | EVQLVESGGGLVQPGGSLTLSCVASGSTSPINAIGWYRRAPG |
| (DLL3) | KQRELVAGISSDGSVVTTESVKGRFTISRDNAKNSVYLQMNS |
| (SEQ ID NO: 700) | LRAEDTAVYYCYYFRTGSGSSMGYWGQGTLVTVSS |
| Exemplary anti-target | EVQLVESGGGLVQPGGSLTLSCVASGSITSSNAMGWYRRAPG |
| (DLL3) | KQRELVAGISSDGSHVHQESVKGRFTISRDNAKNSVYLQMNS |
| (SEQ ID NO: 701) | LRAEDTAVYYCYYFTTVTGSSMSYWGQGTLVTVSS |
| Exemplary anti-target | EVQLVESGGGLVQPGGSLTLSCAASRYSVSNLSMAWYRQAPG |
| (DLL3) | KKRELVAGISADGSTVYVESVKGRFTISRDNAKNSVYLQMNS |
| (SEQ ID NO: 702) | LRAEDTAVYYCYAYYWTERRPYWGQGTLVTVSS |
| Exemplary anti-target | EVQLVESGGGLVQPGDSLTLSCAASMSTVSVLSMAWYRQAPG |
| (DLL3) | KKRELVAGISSDGSTVYIDSVKGRFTISRDNAKNSVYLQMNS |
| (SEQ ID NO: 703) | LRAEDTAIYYCYAYSWDDAHPYWGQGTLVTVSS |
| Exemplary anti-target | EVQLVESGGGLVQPGGSLTLSCAASDSYVSLLSMAWYRQAPG |
| (DLL3) | KKRELVAGISVDGSTHYVASVKGRFTISRDNAKNSVYLQMNS |
| (SEQ ID NO: 704) | LRAEDTAVYYCYAYRWMTRLTYWGQGTLVTVSS |
| Exemplary anti-target | EVQLVESGGGLVQPGGSLTLSCAASDSAVSVLSIAWYRQAPG |
| (DLL3) | KKRELVAGISTDGSKHYIDSVKGRFTISRDNAKNSVYLQMNS |
| (SEQ ID NO: 705) | LRAEDTAVYYCYAYDWADAQPYWGQGTLVTVSS |
| Exemplary anti-target | EVQLVESGGGLVQPGGSLTLSCAASHSSVTSLSLAWYRQAPG |
| (DLL3) | KKRELVAGISYDGSKYYAESVKGRFTISRDNAKNSVYLQMNS |
| (SEQ ID NO: 706) | LRAEDTAVYYCYAYSWTDRLPYWGQGTLVTVSS |
| Exemplary anti-target | EVQLVESGGGLVQPGGSLTLSCAASDSVVKFLSMAWYRQAPG |
| (DLL3) | KKRELVAGISANGSRTYMESVKGRFTISRDNAKNSVYLQMNS |
| (SEQ ID NO: 707) | LRAEDTAVYYCYAYRWATRLPYWGQGTLVTVSS |
| Exemplary anti-target | EVQLVESGGGLVQPGGSLTLSCAASDPSVWNLSMAWYRQAPG |
| (DLL3) | KKRELVAGISPDGSTDYVDSVKGRFTISRDNAKNSVYLQMNS |
| (SEQ ID NO: 708) | LRAEDTAVYYCYAYKWSNRLPYWGQGTLVTVSS |
| Exemplary anti-target | EVQLVESGGGLVQPGGSLTLSCAASGTSVMLLSLAWYRQAPG |
| (DLL3) | KKRELVAGISPNGSAVYTESVKGRFTISRDNAKNSVYLQMNS |
| (SEQ ID NO: 709) | LRAEDTAVYYCYAYGWKTRQPYWGQGTLVTVSS |
| Exemplary anti-target | EVQLVESGGGLVQPGGSLTLSCAASSSPVSNLSLAWYRQAPG |
| (DLL3) | KKRELVAGISPDGSTAYMESVKGRFTISRDNAKNSVYLQMNS |
| (SEQ ID NO: 710) | LRAEDTAVYYCYAYRWPNRRGYWGQGTLVTVSS |
| Exemplary anti-target | EVQLVESGGGLVQPGGSLTLSCAASWRSVLLLSVAWYRQAPG |
| (DLL3) | KKRELVAGISNDGSTDYIDSVKGRFTISRDNAKNSVYLQMNS |
| (SEQ ID NO: 711) | LRAEDTAVYYCYAYDWTTRQRYWGQGTLVTVSS |
| Exemplary anti-target | EVQLVESGGGLVQPGGSLTLSCAASSSSVQYLSMAWYRQAPG |
| (DLL3) | KKRELVAGISTDGSAVYFDSVKGRFTISRDNAKNSVYLQMNS |
| (SEQ ID NO: 712) | LRAEDTAVYYCYAYNWSYAQPYWGQGTLVTVSS |
| Exemplary anti-target | EVQLVESGGGLVQPGGSLTLSCAASGTSVSLLSLAWYRQAPG |
| (DLL3) | KKRELVAGISTGGSTHYIESVKGRFTISRDNAKNSVYLQMNS |
| (SEQ ID NO: 713) | LRAEDTAVYYCYAYNWTDSLQYWGQGTLVTVSS |
| Exemplary anti-target | EVQLVESGGGLVQPGGSLTLSCAASLSSVSNLSIAWYRQAPG |
| (DLL3) | KKRELVAGISTDGSTVYIDSVKGRFTISRDNAKNSVYLQMNS |
| (SEQ ID NO: 714) | LRAEDTAVYYCYAYSWTTSLPYWGQGTLVTVSS |
| Exemplary anti-target | EVQLVESGGGLVQPGGSLTLSCAASMYSVSFLSMAWYRQAPG |
| (DLL3) | KKRELVAGISNEGSTYYMDSVKGRFTISRDNAKNSVYLQMNS |
| (SEQ ID NO: 715) | LRAEDTAVYYCYAYKWRSRSTYWGQGTLVTVSS |
| Exemplary anti-target | EVQLVESGGGLVQPGGSLTLSCAASKSSVSHLSLAWYRQAPG |
| (DLL3) | KKRELVAGISADGSHVYTNSVKGRFTISRDNAKNSVYLQMNS |
| (SEQ ID NO: 716) | LRAEDTAVYYCYAYSQTTRDPYWGQGTLVTVSS |
| Exemplary anti-target | EVQLVESGGGLVQPGGSLTLSCAASYTSVLDLSIAWYRQAPG |
| (DLL3) | KKRELVAGISDDGSRYYTDSVKGRFTISRDNAKNSVYLQMNS |
| (SEQ ID NO: 717) | LRAEDTAVYYCYAYRWTARDTYWGQGTLVTVSS |
| Exemplary anti-target | EVQLVESGGGLVQPGGSLTLSCAASMSDVSFLSMAWYRQAPG |
| (DLL3) | KKRELVAGISAEGSTLYMESVKGRFTISRDNAKNSVYLQMNS |
| (SEQ ID NO: 718) | LRAEDTAVYYCYAYRWTSRLSYWGQGTLVTVSS |
| Exemplary anti-target | EVQLVESGGGLVQPGGSLTLSCAASESSVSFLSSAWYRQAPG |
| (DLL3) | KKRELVAGISTDGSTVYIDSVKGRFTISRDNAKNSVYLQMNS |
| (SEQ ID NO: 719) | LRAEDTAVYYCYAYSWTTRSRYWGQGTLVTVSS |
| Exemplary anti-target | EVQLVESGGGLVQPGGSLTLSCAASGDSVSLLSMAWYRQAPG |
| (DLL3) | KKRELVAGISANGSTSYIDSVKGRFTISRDNAKNSVYLQMNS |
| (SEQ ID NO: 720) | LRAEDTAVYYCYAYNWTSRYRYWGQGTLVTVSS |
| Exemplary anti-target | EVQLVESGGGLVQPGGSLTLSCAASGSDVWYLSLAWYRQAPG |
| (DLL3) | KKRELVAGISDDGSRHYIESVKGRFTISRDNAKNSVYLQMNS |
| (SEQ ID NO: 721) | LRAEDTAVYYCYAYSWKTRFPYWGQGTLVTVSS |
| Exemplary anti-target | EVQLVESGGGLVQPGGSLTLSCAASKSAVAFLSIAWYRQAPG |
| (DLL3) | KKRELVAGISPDGSTVYIESVKGRFTISRDNAKNSVYLQMNS |
| (SEQ ID NO: 722) | LRAEDTAVYYCYAYSWTTRYPYWGQGTLVTVSS |
| Exemplary anti-target | EVQLVESGGGLVQPGGSLTLSCAASFSAVAYLSMAWYRQAPG |
| (DLL3) | KKRELVAGISDDGSTVYVDSVKGRFTISRDNAKNSVYLQMNS |
| (SEQ ID NO: 723) | LRAEDTAVYYCYAYEWTNALPYWGQGTLVTVSS |
| Exemplary anti-target | EVQLVESGGGLVQPGGSLTLSCAASVYSVYDLSTAWYRQAPG |
| (DLL3) | KKRELVAGISDDGSTVYFDSVKGRFTISRDNAKNSVYLQMNS |
| (SEQ ID NO: 724) | LRAEDTAVYYCYAYSWITRSPYWGQGTLVTVSS |
| Exemplary anti-target | EVQLVESGGGLVQPGGSLTLSCAASGDSVSFLSMAWYRQAPG |
| (DLL3) | KKRELVAGISDEGSTVYIGSVKGRFTISRDNAKNSVYLQMNS |
| (SEQ ID NO: 725) | LRAEDTAVYYCYAYSWTTRRQYWGQGTLVTVSS |
| Exemplary anti-target | EVQLVESGGGLVQPGGSLTLSCAASSSSVSLLSLAWYRQAPG |
| (DLL3) | KKRELVAGISDDGSIVYMDSVKGRFTISRDNAKNSVYLQMNS |
| (SEQ ID NO: 726) | LRAEDTAVYYCYAYSWITRSPYWGQGTLVTVSS |
| Exemplary anti-target | EVQLVESGGGLVQPGGSLTLSCAASADSVSFLSIAWYRQAPG |
| (DLL3) | KKRELVAGISDDGSKHYFDSVKGRFTISRDNAKNSVYLQMNS |
| (SEQ ID NO: 727) | LRAEDTAVYYCYAYRWEESRQYWGQGTLVTVSS |
| Exemplary anti-target | EVQLVESGGGLVQPGGSLTLSCAASASSVTLLSIAWYRQAPG |
| (DLL3) | KKRELVAGISTDGSTDYLHSVKGRFTISRDNAKNSVYLQMNS |
| (SEQ ID NO: 728) | LRAEDTAVYYCYAYTWTTRLPYWGQGTLVTVTS |
| Exemplary anti-target | EVQLVESGGGLVQPGGSLTLSCAASADSVSFLSIAWYRQAPG |
| (DLL3) | KKRELVAGISDDGSKHYFDSVKGRFTISRDNAKNSVYLQMNS |
| (SEQ ID NO: 729) | LRAEDTAVYYCYAYRWEESRQYWGQGTLVTVSS |
| Exemplary anti-target | EVQLVESGGGLVQPGGSLTLSCAASGTSVWLLSMAWYRQAPG |
| (DLL3) | KKRELVAGISYDGSTVYVESVKGRFTISRDNAKNSVYLQMNS |
| (SEQ ID NO: 730) | LRAEDTAVYYCYAYSWTTRQPYWGQGTLVTVSS |
| Exemplary anti-target | EVQLVESGGGLVQPGGSLTLSCAASGSSVSILSIAWYRQAPG |
| (DLL3) | KKRELVAGISDDGSTVYIDSVKGRFTISRDNAKNSVYLQMNS |
| (SEQ ID NO: 731) | LRAEDTAVYYCYAYVWGTRLPYWGQGTLVTVSS |
| Exemplary anti-target | EVQLVESGGGLVQPGGSLTLSCAASGTAVSNLSIAWYRQAPG |
| (DLL3) | KKRELVAGISDDGSTVYVDSVKGRFTISRDNAKNSVYLQMNS |
| (SEQ ID NO: 732) | LRAEDTAVYYCYAYEWTNALPYWGQGTLVTVSS |
| Exemplary anti-target | EVQLVESGGGLVQPGGSLTLSCAASGSAVSMLSLAWYRQAPG |
| (DLL3) | KKRELVAGISDDGSQVYIDSVKGRFTISRDNAKNSVYLQMNS |
| (SEQ ID NO: 733) | LRAEDTAVYYCYAYRWEDALTYWGQGTLVTVSS |
| Exemplary anti-target | EVQLVESGGGLVQPGGSLTLSCAASGMTVFFLSMAWYRQAPG |
| (DLL3) | KKRELVAGISVDGSTVYSDSVKGRFTISRDNAKNSVYLQMNS |
| (SEQ ID NO: 734) | LRAEDTAVYYCYAYSWTTRYPYWGQGTLVTVSS |
| Exemplary anti-target | EVQLVESGGGLVQPGGSLTLSCAASQYSVTFLSVAWYRQAPG |
| (DLL3) | KKRELVAGISDDGSNVYIDSVKGRFTISRDNAKNSVYLQMNS |
| (SEQ ID NO: 735) | LRAEDTAVYYCYAYSWIDSLRYWGQGTLVTVSS |
| Exemplary anti-target | EVQLVESGGGLVQPGGSLTLSCAASGETVSFLSLAWYRQAPG |
| (DLL3) | KKRELVAGISTDGSTVYFVSVKGRFTISRDNAKNSVYLQMNS |
| (SEQ ID NO: 736) | LRAEDTAVYYCYAYSWTTPRAYWGQGTLVTVSS |
| Exemplary anti-target | QVQLQESGGGLVQAGGSLRLSCAASGSIAYIYTMDWYRQAPG |
| (EGFR) | KQRELVATSTRDGNVDYAESVKGRFTISRDNAKNTVYLQMNS |
| (SEQ ID NO: 737) | LKPEDTAVYYCNADLRTAVDLIRANYWGQGTQVTVSS |
| Exemplary anti-target | QVQLQESGGGLVQAGDSLRLSCVVSGRTDSWYVMGWFRQAPG |
| (EGFR) | KDREFVAGVSWSYGNTYYADSVKGRFTASRDNAKNTAYLQMN |
| (SEQ ID NO: 738) | SLNAEDTAVYYCAARVSREVIPTRWDLYNYWGQGTQVTVSS |
| Exemplary anti-target | QVQLQESGGGSVQPGGSLRVSCVVSRTIISINAMTWYHQAPG |
| (EGFR) | KRRELVAIITSGGETNYADSVKGRFTISRDNAKNTAYLQMNN |
| (SEQ ID NO: 739) | LKPEDTGVYYCNVVPPLGSWGQGTQVTVSS |
| Exemplary anti-target | QVQLQESGGGRVQAGGSLRLSCSASARTLRLYAVGWFRQAPG |
| (EGFR) | KEREFVAGIGRSERTYYTDSVKGRFTLSRDNAKNTVFLEMND |
| (SEQ ID NO: 740) | LEPEDTAVYFCALTFQTTDMVDVPTTQHEYDYWGRGTQVTVS |
| | S |
| Exemplary anti-target | QVQLQESGGGLVQAGGSLRLSCAASGSIAYIYTMDWYRQAPG |
| (EGFR) | KQRELVATSTHDGNTDYADSVKGRFTISRDNVKNTVYLQMNS |
| (SEQ ID NO: 741) | LKPEDTAVYYCNADLRTAVDLIRANYWGQGTQVTVSS |
| Exemplary anti-target | QVQLQESGGGLVQPGGSLRLSCAASGSIAYIYTMDWYRQAPG |
| (EGFR) | KQRELVATSTRDGNTDYADSVKGRFTISRDNAKDTVYLQMNS |
| (SEQ ID NO: 742) | LKPEDTAVYYCNADLRTAVDLIRANYWGQGTQVTVSS |
| Exemplary anti-target | QVQLQESGGGLVQAGGSLTLSCAASGRYQMAWFRQAPEKERE |
| (EGFR) | FVGTISSGDSTWYTNSVKGRFAISRDSARNTVYLQMNDLKPE |
| (SEQ ID NO: 743) | DTAIYYCAAALYYRDSRRAADYPYWGQGTQVTVSS |
| Exemplary anti-target | QVQLQESGGGRVQAGESLRLSCSTSTRTLKLYAVGWFRQAPG |
| (EGFR) | KERDFVAGIGRSERIYYIDSVKGRFTLSRDNAKNTVFLEMND |
| (SEQ ID NO: 744) | LEPEDTAVYFCAATFQTSDNVGVPTVQHEYDYWGQGTQVTVS |
| | S |
| Exemplary anti-target | QVQLQESGGGLVQAGGSLRLSCAASGSIAYIYTMDWYRQAPG |
| (EGFR) | KQRELVATSTHDGNTDYADSVKGRFTISRDNAKNTVTLQMNS |
| (SEQ ID NO: 745) | LKPEDTAVYYCNADLRTAVDLIRANYWGQGTQVTVSS |
| Exemplary anti-target | QVQLQESGGGLVQAGGSLRLSCAASGSIAYIYTMDWYRQAPG |
| (EGFR) | KQRELVATFTRDGNTDYADSVKGRFTISRDNAKNTVYLQMNS |
| (SEQ ID NO: 746) | LKPEDTAVYYCNTDLRTAVDLIRANYWGQGTQVTVSS |
| Exemplary anti-target | QVQLQESGGGLVQAGGSLRLSCAASGSIAYIYTMDWYRQAPG |
| (EGFR) | KQRELVATSTHDGNTDYADSLKGRFTISRDNAKNTVYLQMNS |
| (SEQ ID NO: 747) | LKPEDTAVYYCNADLRTAVDLIRANYWGQGTQVTVSS |
| Exemplary anti-target | QVQLQESGGGSVQAGGSLRLSCAASGRYQMAWFRQAPEKERE |
| (EGFR) | FVGTISSGDSTWYTNSLKGRFAISRDSARDTVYLQMNDLKPE |
| (SEQ ID NO: 748) | DTAVYYCAAALYYRDSRRAADYPYWGQGTQVTVSS |
| Exemplary anti-target | QVQLQESGGGLVQTGGSLRLSCAVSGSIVTINAMTWYRQAPG |
| (EGFR) | KRRELVAIITSGGETNYADSVKGRFTISRDNAKNTAHLQMNS |
| (SEQ ID NO: 749) | LNPEDTGVYYCNVVPPLGSWGQGTQVTVSS |
| Exemplary anti-target | QVQLQESGGGLVQTGGSLRLSCAVSRSIVSIKSMTWYRQAPG |
| (EGFR) | KRRELVALITSGGETNYSDSVKGRFTISRDNAKNTVYLQMNS |
| (SEQ ID NO: 750) | LKPEDTGVYYCNVVPPLGSWGQGTQVTVSS |
| Exemplary anti-target | QVQLQESGGGLVETGGSLRLSCAGSGSTFRHHAMAWFRQTPG |
| (EGFR) | KEREFVSAINDHGDRTKYLDSVRGRFTISRDNTDNMVYLQMT |
| (SEQ ID NO: 751) | DLRPEDTANYSCAAGPLVDYLETTPLVYTYWGHGTQVTVSS |
| Exemplary anti-target | QVQLQESGGGLVQAGGSLRLSCAASGSIAYIYTMDWYRQAPG |
| (EGFR) | KQRELVATSTHDGNTDYADSVKGRFTISRDNAKNTVYLQMNS |
| (SEQ ID NO: 752) | LKPEDTAVYYCNADLRTAVDLIRANYWGQGTQVTVSS |
| Exemplary anti-target | QVQLQESGGGSVQAGGSLTLSCAASGSIAYIYTMDWYRQAPG |
| (EGFR) | KQRELVATSTHDGNTDYADSVKGRFTISRDNAKNTVYLQMNS |
| (SEQ ID NO: 753) | LKPEDTAVYYCNADLRTAVDLIRANYWGQGTQVTVSS |
| Exemplary anti-target | QVQLQESGGGLVQPGGSLRLSCTASVSIFSVNAVDWYRQSPG |
| (EGFR) | KERELVAIMTSDGSTNYGDSVKGRFTISRDNAKNTVYLQMNN |
| (SEQ ID NO: 754) | LKPEDTAVYYCNTVPPRYWGQGTQVTVSS |
| Exemplary anti-target | QVQLQESGGGLVQPGGSLRLSCAASGSIAYIYTMDWYRQAPG |
| (EGFR) | KQRELVATSTRDGNIDYADSVKGRFTISRDSAKNTVYLQMSS |
| (SEQ ID NO: 755) | LKPEDTAVYYCNADLRTAVDLIRANYWGQGTQVTVSS |
| Exemplary anti-target | QVQLQESGGGLVQAGGSLTLSCAASGRYQLAWFRQAPEKVRE |
| (EGFR) | FVGTISSGDSTWYTNSVKGRFAISRDSARNTVYLQMNDLKPE |
| (SEQ ID NO: 756) | DTAVYYCAAALYYRDSRRAADYPYWGQGTQVTVSS |
| Exemplary anti-target | QVQLQESGGGSVQAGGSLRLSCAASGRYHMAWFRQAPEKERE |
| (EGFR) | FVGTISSGDSTWYTNSVKGRFAISRDSARNTAYLQMNDLKPE |
| (SEQ ID NO: 757) | DTAVYYCAAALYYGDSRRAADYPYWGQGTQVTVSS |
| Exemplary anti-target | QVQLQESGGGSVQAGGSLRLSCAASGSIAYIYTMDWYRQAPG |
| (EGFR) | KQRELVATSTHDGNTDYTDSVKGRFTISRDNAKNTVYLQMNS |
| (SEQ ID NO: 758) | LKPEDTAVYYCNADLRTAVDLIRANYWGQGTQVTVSS |
| Exemplary anti-target | QVQLQESGGGLVQAGGSLRLSCAASGSIAYIYTMDWYRQTPG |
| (EGFR) | KQRELVATSTRDANTDYAGSVKGRFTISRDNAKDTVYLQMNS |
| (SEQ ID NO: 759) | LKPEDTAVYYCHADLRTAVDLIRANYWGQGTQVTVSS |
| Exemplary anti-target | QVQLQESGGGLVQAGGSLRLSCAASGRYNMAWFRQAPEKERE |
| (EGFR) | FVGTITSADSTWYTNSVKGRFAITQDSARNTVYLQMNDLKPE |
| (SEQ ID NO: 760) | DTAVYYCAAALYYGDSRRAADYPYWGQGTQVTVSS |
| Exemplary anti-target | QVQLQESGGGLVQPGGSLRLSCAASGRYQMAWFRQAPEKERE |
| (EGFR) | FVGTISSGDSTWYTNSVKGRFAISRDSARTTVYLQMNDLKPE |
| (SEQ ID NO: 761) | DTAVYYCAAALYYRDSWRAADYPYWGQGTQVTVSS |
| Exemplary anti-target | QVQLQESGGGLVQPGESLRLSCAATGRYHLAWFRQAPEKERE |
| (EGFR) | FVGTITSADSTWYTNSVKGRFAITRDSARNTVYLQMNDLKPE |
| (SEQ ID NO: 762) | DTAVYYCAAALYYGDSRRAADYPYWGQGTQVTVSS |
| Exemplary anti-target | QVQLQESGGGLVQAGGSLKLSCADSGRSFSNYIMGWFRQAPG |
| (EGFR) | KEREFVAGLGWSPGNTYYADSVKGRFTISRDNAKNMVYLQMN |
| (SEQ ID NO: 763) | SLNPEDTAVYYCAARRGDVIYTTPWNYVYWGQGTQVTVSS |
| Exemplary anti-target | QVQLQESGGGLVQAGGSLRLSCAASGSIAYIYTMDWYRQAPG |
| (EGFR) | KQRELVATSTHDGNTDYADSVKGRFTISRDNAKNTVYLQMNS |
| (SEQ ID NO: 764) | LKPEDTAVYYCNADLRTPVDLIRANYWGQGTQVTVSS |
| Exemplary anti-target | QVQLQESGGGSVQAGGSLRLSCAAPGRYQMAWFRQAPEKERE |
| (EGFR) | FVGTISSGDSTWYTNSVKGRFAISRDSARNTVYLQMNDLKSE |
| (SEQ ID NO: 765) | DTAVYYCAAALYYRDSRRAIDYPYWGQGTQVTVSS |
| Exemplary anti-target | QVQLQESGGGSVQAGGSLRLSCAASGLTFSSYAMAWFRQAPG |
| (EGFR) | KQRELVARITSGGTTDYADSVKGRFTISRDNAKNTMYLQMNS |
| (SEQ ID NO: 766) | LKPEDTAVYYCAADLTYRNLLLKLPHYWGQGTQVTVSS |
| Exemplary anti-target | QVQLQESGGGLVQAGGSLRLSCAASGNIAYIYTMDWYRQAPG |
| (EGFR) | KQRELVATSTHDGSTDYADSVKGRFTISRDNAKNTVYLQMNS |
| (SEQ ID NO: 767) | LKPEDTAVYYCNADLRTPVDLIRANYWGQGTQVTVSS |
| Exemplary anti-target | QVQLQESGGGLVQPGGSLRLSCAASGSIAYIYTMDWYRQAPG |
| (EGFR) | KQRELVATSTWDGNTDYADSVKGRFTISRDNAKNTVYLQMNS |
| (SEQ ID NO: 768) | LKPEDTAVYYCNADLRTAVDLIRANYWGQGTQVTVSS |
| Exemplary anti-target | QVQLQESGGGLVQAGGSLRLSCAASGSIAYIYTMDWYRQAPG |
| (EGFR) | KQRELVATSTHDGNTDYADSVKGRFTISRDNAKNTVYLQMSS |
| (SEQ ID NO: 769) | LKPDDTAVYYCNADLRTAVDLIRANYWGQGTQVTVSS |
| Exemplary anti-target | QVQLQESGGGLVQAGESLSLSCAASGNDEVITDMHWYRQAPG |
| (EGFR) | KQREWVATITRFATTNYADSVKGRFTISRDNAKNTWYLQMNS |
| (SEQ ID NO: 770) | LKPDDTAVYYCKAIGLRGVPDVNRQFEVWGQGTQVTVSS |
| Exemplary anti-target | QVQLQESGGGLVQAGGSLRLSCAASGAIAYIYGMGWYRQAPG |
| (EGFR) | NQRELVAAISSGGSTDYADSVKGRFTISRDNAKNTVYLQMSS |
| (SEQ ID NO: 771) | LKPEDTAVYYCNADVRTSRNLVRSDYWGQGTQVTVSS |
| Exemplary anti-target | QVQLQESGGGLVQAGGSLRLSCAASGRYHTAWFRQAPEKERE |
| (EGFR) | FVGTISSGDSTWYTNSVKGRFAISRDSARNTVYLQMNDLKPE |
| (SEQ ID NO: 772) | DTAVYYCAAALYYGDSRRAGDYPYWGQGTQVTVSS |
| Exemplary anti-target | QVQLQESGGGLVQAGGSLRLSCAASGNIAYIYTMNWYRQAPG |
| (EGFR) | KQRELVATSTHAGNTDYADSVKGRFTISRDNAKNTVYLQMNS |
| (SEQ ID NO: 773) | LKPEDTAVYYCNVDLRTAVDLIRANYWGQGTQVTVSS |
| Exemplary anti-target | QVQLQESGGGLVQPGGSLRLSCAASGNIAYIYTMGWYRQAPG |
| (EGFR) | KQRELVATSTHDGNSDYADSVKGRFTISRDNAKNTVYLQMNT |
| (SEQ ID NO: 774) | LKPDDTAVYYCNADLRTPVDRIRGNFWGQGTQVTVSS |
| Exemplary anti-target | EVQLLESGGGLVQPGGSLTLSCAASGSIAYIYTMDWYRQAPG |
| (EGFR) | KQRELVATSTRDGNVDYADSVKGRFTISRDNSKNTLYLQMNS |
| (SEQ ID NO: 775) | LRAEDTAVYYCNADLRTAVDLIRANYWGLGTQVTVSS |
| Exemplary anti-target | EVQLLESGGGLVQPGGSLTLSCAASGRTDSWYVMGWFRQAPG |
| (EGFR) | KDREFVAGVSWSYGNTYYADSVKGRFTISRDNSKNTLYLQMN |
| (SEQ ID NO: 776) | SLRAEDTAVYYCAARVSREVIPTRWDLYNYWGLGTQVTVSS |
| Exemplary anti-target | EVQLLESGGGLVQPGGSLTLSCAASARTLRLYAVGWFRQAPG |
| (EGFR) | KEREFVAGIGRSERTYYTDSVKGRFTISRDNSKNTLYLQMNS |
| (SEQ ID NO: 777) | LRAEDTAVYYCALTFQTTDMVDVPTTQHEYDYWGLGTQVTVS |
| | S |
| Exemplary anti-target | EVQLLESGGGLVQPGGSLTLSCAASGSIVTINAMTWYRQAPG |
| (EGFR) | KRRELVAIITSGGETNYADSVKGRFTISRDNSKNTLYLQMNS |
| (SEQ ID NO: 778) | LRAEDTAVYYCNVVPPLGSWGLGTQVTVSS |
| Exemplary anti-target | EVQLLESGGGLVQPGGSLTLSCAASGRYHMAWFRQAPGKERE |
| (EGFR) | FVGTISSGDSTWYTNSVKGRFTISRDNSKNTLYLQMNSLRAE |
| (SEQ ID NO: 779) | DTAVYYCAAALYYGDSRRAADYPYWGLGTQVTVSS |
| Exemplary anti-target | EVQLLESGGGLVQPGGSLTLSCAASGSTFRHHAMAWFRQTPG |
| (EGFR) | KEREFVSAINDHGDRTKYLDSVKGRFTISRDNSKNTLYLQMN |
| (SEQ ID NO: 780) | SLRAEDTAVYYCAAGPLVDYLETTPLVYTYWGLGTQVTVSS |
| Exemplary anti-target | EVQLLESGGGLVQPGGSLTLSCAASGRSFSNYIMGWFRQAPG |
| (EGFR) | KEREFVAGLGWSPGNTYYADSVKGRFTISRDNSKNTLYLQMN |
| (SEQ ID NO: 781) | SLRAEDTAVYYCAARRGDVIYTTPWNYVYWGLGTQVTVSS |
| Exemplary anti-target | EVQLLESGGGLVQPGGSLTLSCAASGLTFSSYAMAWFRQAPG |
| (EGFR) | KQRELVARITSGGTTDYADSVKGRFTISRDNSKNTLYLQMNS |
| (SEQ ID NO: 782) | LRAEDTAVYYCAADLTYRNLLLKLPHYWGLGTQVTVSS |
| Exemplary anti-target | EVQLLESGGGLVQPGGSLTLSCAASVSIFSVNAVDWYRQSPG |
| (EGFR) | KERELVAIMTSDGSTNYDDSVKGRFTISRDNSKNTLYLQMNS |
| (SEQ ID NO: 783) | LRAEDTAVYYCNTVPPRYWGLGTQVTVSS |
| Exemplary anti-target | EVQLLESGGGLVQPGGSLTLSCAASGNDFVITDMHWYRQAPG |
| (EGFR) | KQREWVATITRFATTNYADSVKGRFTISRDNSKNTLYLQMNS |
| (SEQ ID NO: 784) | LRAEDTAVYYCKAIGLRGVPDVNRQFEVWGLGTQVTVSS |
| Exemplary anti-target | EVQLLESGGGLVQPGGSLTLSCAASGAIAYIYGMGWYRQAPG |
| (EGFR) | KQRELVAAISSGGSTDYADSVKGRFTISRDNSKNTLYLQMNS |
| (SEQ ID NO: 785) | LRAEDTAVYYCNADVRTSRNLVRSDYWGLGTQVTVSS |
| Exemplary EGFR | EVQLVESGGGLVQPGNSLRLSCAASGFTFSKFGMSWVRQGGG |
| ProTriTAC containing | GGLDGNEEPGGLEWVSSISGSGRDTLYADSVKGRFTISRDNA |
| linker sequence L001 | KTTLYLQMNSLRPEDTAVYYCTIGGSLSVSSQGTLVTVSS |
| (identified by residues | GGGGKPLGLQARVVGGGGT |
| in bold and italics) | QTVVTQEPSLTVSPGGTVTLTCASSTGAVTSGNYPNWVQQKP |
| and masking sequence | GQAPRGLIGGTKFLVPGTPARFSGSLLGGKAALTLSGVQPED |
| M027 (identified by | EAEYYCTLWYSNRWVFGGGTKLTVLGGGGSGGGGSGGGGSEV |
| residues that are bold | QLVESGGGLVQPGGSLKLSCAASGFTFNKYAINWVRQAPGKG |
| and underlined) | LEWVARIRSKYNNYATYYADQVKDRFTISRDDSKNTAYLQMN |
| (SEQ ID No. 786) | NLKTEDTAVYYCVRHANFGNSYISYWAYWGQGTLVTVSSGGG |
| | GSGGGSEVQLLESGGGLVQPGGSLTLSCAASGRTDSWYVMGW |
| | FRQAPGKDREFVAGVSWSYGNTYYADSVKGRFTISRDNSKNT |
| | LYLQMNSLRAEDTAVYYCAARVSREVIPTRWDLYNYWGLGTQ |
| | VTVSSHHHHHH |
| Exemplary EGFR | EVQLVESGGGLVQPGNSLRLSCAASGFTFSKFGMSWVRQGGG |
| ProTriTAC containing | GGLDGNEEPGGLEWVSSISGSGRDTLYADSVKGRFTISRDNA |
| linker sequence L040 | KTTLYLQMNSLRPEDTAVYYCTIGGSLSVSSQGTLVTVSSGG |
| (identified by residues | GGPQASTGRSGGGGGGT |
| in bold and italics) | QTVVTQEPSLTVSPGGTVTLTCASSTGAVTSGNYPNWVQQKP |
| and masking sequence | GQAPRGLIGGTKFLVPGTPARFSGSLLGGKAALTLSGVQPED |
| M027 (identified by | EAEYYCTLWYSNRWVFGGGTKLTVLGGGGSGGGGSGGGGSEV |
| residues that are bold | QLVESGGGLVQPGGSLKLSCAASGFTFNKYAINWVRQAPGKG |
| and underlined) | LEWVARIRSKYNNYATYYADQVKDRFTISRDDSKNTAYLQMN |
| (SEQ ID No. 787) | NLKTEDTAVYYCVRHANFGNSYISYWAYWGQGTLVTVSSGGG |
| | GSGGGSEVQLLESGGGLVQPGGSLTLSCAASGRTDSWYVMGW |
| | FRQAPGKDREFVAGVSWSYGNTYYADSVKGRFTISRDNSKNT |
| | LYLQMNSLRAEDTAVYYCAARVSREVIPTRWDLYNYWGLGTQ |
| | VTVSSHHHHHH |
| Exemplary EGFR | EVQLVESGGGLVQPGNSLRLSCAASGFTFSKFGMSWVRQGGG |
| ProTriTAC containing | GGLDGNEEPGGLEWVSSISGSGRDTLYADSVKGRFTISRDNA |
| linker sequence L041 | KTTLYLQMNSLRPEDTAVYYCTIGGSLSVSSQGTLVTVSS |
| (identified by residues | GGGGPQGSTGRAAGGGGGT |
| in bold and italics) | QTVVTQEPSLTVSPGGTVTLTCASSTGAVTSGNYPNWVQQKP |
| and masking sequence | GQAPRGLIGGTKFLVPGTPARFSGSLLGGKAALTLSGVQPED |
| M027 (identified by | EAEYYCTLWYSNRWVFGGGTKLTVLGGGGSGGGGSGGGGSEV |
| residues that are bold | QLVESGGGLVQPGGSLKLSCAASGFTFNKYAINWVRQAPGKG |
| and underlined) | LEWVARIRSKYNNYATYYADQVKDRFTISRDDSKNTAYLQMN |
| (SEQ ID No. 788) | NLKTEDTAVYYCVRHANFGNSYISYWAYWGQGTLVTVSS |
| | GGGGSGGGSEVQLLESGGGLVQPGGSLTLSCAASGRTDSWYV |
| | MGWFRQAPGKDREFVAGVSWSYGNTYYADSVKGRFTISRDNS |
| | KNTLYLQMNSLRAEDTAVYYCAARVSREVIPTRWDLYNYWGL |
| | GTQVTVSSHHHHHH |
| Exemplary EGFR | EVQLVESGGGLVQPGNSLRLSCAASGFTFSKFGMSWVRQGGG |
| ProTriTAC containing | GGLDGNEEPGGLEWVSSISGSGRDTLYADSVKGRFTISRDNA |
| linker sequence L042 | KTTLYLQMNSLRPEDTAVYYCTIGGSLSVSSQGTLVTVSSGG |
| (identified by residues | GGPPASSGRAGGGGGT |
| in bold and italics) | QTVVTQEPSLTVSPGGTVTLTCASSTGAVTSGNYPNWVQQKP |
| and masking sequence | GQAPRGLIGGTKFLVPGTPARFSGSLLGGKAALTLSGVQPED |
| M027 (identified by | EAEYYCTLWYSNRWVFGGGTKLTVLGGGGSGGGGSGGGGSEV |
| residues that are bold | QLVESGGGLVQPGGSLKLSCAASGFTFNKYAINWVRQAPGKG |
| and underlined) | LEWVARIRSKYNNYATYYADQVKDRFTISRDDSKNTAYLQMN |
| (SEQ ID No. 789) | NLKTEDTAVYYCVRHANFGNSYISYWAYWGQGTLVTVSS |
| | GGGGSGGGSEVQLLESGGGLVQPGGSLTLSCAASGRTDSWYV |
| | MGWFRQAPGKDREFVAGVSWSYGNTYYADSVKGRFTISRDNS |
| | KNTLYLQMNSLRAEDTAVYYCAARVSREVIPTRWDLYNYWGL |
| | GTQVTVSSHHHHHH |
| Exemplary EGFR | EVQLVESGGGLVQPGNSLRLSCAASGFTFSKFGMSWVRQGGG |
| ProTriTAC containing | GGLDGNEEPGGLEWVSSISGSGRDTLYADSVKGRFTISRDNA |
| linker sequence L045 | KTTLYLQMNSLRPEDTAVYYCTIGGSLSVSSQGTLVTVSSGG |
| (identified by residues | GGPIPVQGRAHGGGGTQTVVTQEPSLTVSPGGTVTLTCASST |
| in bold and italics) | GAVTSGNYPNWVQQKPGQAPRGLIGGTKFLVPGTPARFSGSL |
| and masking sequence | LGGKAALTLSGVQPEDEAEYYCTLWYSNRWVFGGGTKLTVLG |
| M027 (identified by | GGGSGGGGSGGGGSEVQLVESGGGLVQPGGSLKLSCAASGET |
| residues that are bold | FNKYAINWVRQAPGKGLEWVARIRSKYNNYATYYADQVKDRF |
| and underlined) | TISRDDSKNTAYLQMNNLKTEDTAVYYCVRHANFGNSYISYW |
| (SEQ ID No. 790) | AYWGQGTLVTVSSGGGGSGGGS |
| | EVQLLESGGGLVQPGGSLTLSCAASGRTDSWYVMGWFRQAPG |
| | KDREFVAGVSWSYGNTYYADSVKGRFTISRDNSKNTLYLQMN |
| | SLRAEDTAVYYCAARVSREVIPTRWDLYNYWGLGTQVTVSSH |
| | HHHHH |
| Exemplary EGFR | EVQLVESGGGLVQPGNSLRLSCAASGFTFSKFGMSWVRQGGG |
| ProTriTAC containing a | GGLDGNEEPGGLEWVSSISGSGRDTLYADSVKGRFTISRDNA |
| non cleavable linker | KTTLYLQMNSLRPEDTAVYYCTIGGSLSVSSQGTLVTVSSGG |
| sequence (identified by | GGSGGGGSGGVVGGGGTQTVVTQEPSLTVSPGGTVTLTCASS |
| residues in bold and | TGAVTSGNYPNWVQQKPGQAPRGLIGGTKFLVPGTPARFSGS |
| italics) and masking | LLGGKAALTLSGVQPEDEAEYYCTLWYSNRWVFGGGTKLTVL |
| sequence | GGGGSGGGGSGGGGSEVQLVESGGGLVQPGGSLKLSCAASGF |
| M027 (identified by | TFNKYAINWVRQAPGKGLEWVARIRSKYNNYATYYADQVKDR |
| residues that are bold | FTISRDDSKNTAYLQMNNLKTEDTAVYYCVRHANFGNSYISY |
| and underlined) | WAYWGQGTLVTVSS |
| (SEQ ID No. 791) | GGGGSGGGSEVQLLESGGGLVQPGGSLTLSCAASGRTDSWYV |
| | MGWFRQAPGKDREFVAGVSWSYGNTYYADSVKGRFTISRDNS |
| | KNTLYLQMNSLRAEDTAVYYCAARVSREVIPTRWDLYNYWGL |
| | GTQVTVSSHHHHHH |
| Exemplary GFP TriTAC | QVQLVESGGALVQPGGSLRLSCAASGFPVNRYSMRWYRQAPG |
| Sequence | KEREWVAGMSSAGDRSSYEDSVKGRFTISRDDARNTVYLQMN |
| (SEQ ID No. 792) | SLKPEDTAVYYCNVNVGFEYWGQGTQVTVSSGGGGSGGGSEV |
| | QLVESGGGLVQPGNSLRLSCAASGFTFSKFGMSWVRQAPGKG |
| | LEWVSSISGSGRDTLYADSVKGRFTISRDNAKTTLYLQMNSL |
| | RPEDTAVYYCTIGGSLSVSSQGTLVTVSSGGGGSGGGSEVQL |
| | VESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSSGGGGS |
| | GGGGSGGGGSQTVVTQEPSLTVSPGGTVTLTCASSTGAVTSG |
| | NYPNWVQQKPGQAPRGLIGGTKFLVPGTPARFSGSLLGGKAA |
| | LTLSGVQPEDEAEYYCTLWYSNRWVFGGGTKLTVLHHHHHH |
| Exemplary EGFR TriTAC | EVQLLESGGGLVQPGGSLTLSCAASGRTDSWYVMGWFRQAPG |
| Sequence | KDREFVAGVSWSYGNTYYADSVKGRFTISRDNSKNTLYLQMN |
| (SEQ ID No. 793) | SLRAEDTAVYYCAARVSREVIPTRWDLYNYWGLGTQVTVSSG |
| | GGGSGGGSEVQLVESGGGLVQPGNSLRLSCAASGFTFSKFGM |
| | SWVRQAPGKGLEWVSSISGSGRDTLYADSVKGRFTISRDNAK |
| | TTLYLQMNSLRPEDTAVYYCTIGGSLSVSSQGTLVTVSSGGG |
| | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH |
| Exemplary anti-CD3 SCFv | QTVVTQEPSLTVSPGGTVTLTCASSTGAVTSGNYPNWVQQKP |
| (SEQ ID NO: 794) | GQAPRGLIGGTKFLVPGTPARFSGSLLGGKAALTLSGVQPED |
| | EAEYYCTLWYSNRWVFGGGTKLTVLGGGGSGGGGSGGGGSEV |
| | QLVESGGGLVQPGGSLKLSCAASGFTFNKYAINWVRQAPGKG |
| | LEWVARIRSKYNNYATYYADQVKDRFTISRDDSKNTAYLQMN |
| | NLKTEDTAVYYCVRHANFGNSYISYWAYWGQGTLVTVSS |
| SEQ ID No: 796 | EVQLVESGGGLVQPGNSLRLSCAASGFTFSKFGMSWVRQGGG |
| | GGLDGNEEPGGLEWVSSISGSGRDTLYADSVKGRFTISRDNA |
| | KTTLYLQMNSLRPEDTAVYYCTIGGSLSVSSQGTLVTVSSGG |
| | GGKPLGLQARVVGGGGT |
| SEQ ID No: 797 | EVQLVESGGGLVQPGNSLRLSCAASGFTFSKFGMSWVRQGGG |
| | GGLDGNEEPGGLEWVSSISGSGRDTLYADSVKGRFTISRDNA |
| | KTTLYLQMNSLRPEDTAVYYCTIGGSLSVSSQGTLVTVSSGG |
| | GGPQASTGRSGGGGGGT |
| SEQ ID No: 798 | EVQLVESGGGLVQPGNSLRLSCAASGFTFSKFGMSWVRQGGG |
| | GGLDGNEEPGGLEWVSSISGSGRDTLYADSVKGRFTISRDNA |
| | KTTLYLQMNSLRPEDTAVYYCTIGGSLSVSSQGTLVTVSSGG |
| | GGPQGSTGRAAGGGGGT |
| SEQ ID No: 799 | EVQLVESGGGLVQPGNSLRLSCAASGFTFSKFGMSWVRQGGG |
| | GGLDGNEEPGGLEWVSSISGSGRDTLYADSVKGRFTISRDNA |
| | KTTLYLQMNSLRPEDTAVYYCTIGGSLSVSSQGTLVTVSSGG |
| | GGPPASSGRAGGGGGT |
| SEQ ID No: 800 | EVQLVESGGGLVQPGNSLRLSCAASGFTFSKFGMSWVRQGGG |
| | GGLDGNEEPGGLEWVSSISGSGRDTLYADSVKGRFTISRDNA |
| | KTTLYLQMNSLRPEDTAVYYCTIGGSLSVSSQGTLVTVSSGG |
| | GGPIPVQGRAHGGGGT |
| Exemplary anti-target | QVQLQESGGGLVQAGGSLRLSCAASGFIFRAASMAWYRQSPG |
| (EpCAM) | NERELVASISSGAFTNYADSVKARFTISRDNAKNTVYLQMNS |
| (SEQ ID NO: 804) EPL90 | LKPEDTAVYFCGATFLRSDGHHTINGQGTQVTVSS |
| Exemplary anti-target | QVQLQESGGGLVQAGGSLRLSCAASGFIFRAASMGWFRQSPG |
| (EpCAM) | NERELVATVSSGDFTNYADSVKGRFTISRDNAKNTVYLQMNS |
| (SEQ ID NO: 805) | LKPEDTAVYFCGATFVRSDGHHTIYGQGTQVTVSS |
| EPL118 | |
| Exemplary anti-target | QVQLQESGGGLVQAGGSLRLSCAASGFIFRAASMDWYRQFPG |
| (EpCAM) | NERESIATISSGGFTNYADSVKGRFTISRDNAKNTVYLQMNS |
| (SEQ ID NO: 806) | LKPEDTAVYFCGATFLRSDGHHTINGQGTQVTVSS |
| EPL138 | |
| Exemplary anti-target | QVQLQESGGGLVQAGGSLRLSCAASGFIFRAASMGWFRQSPG |
| (EpCAM) | NERELVATVSSGGFTNYADSVKGRFTISRDNAKNTVYLQMNS |
| (SEQ ID NO: 807) | LKPEDTAVYFCGATFVRSDGHHTIYGQGTQVTVSS |
| EPL145 | |
| Exemplary anti-target | QVQLQESGGGLVQAGGSLRLSCAASGFIFRAASMDWYRQSPG |
| (EpCAM) | TQPELVATISSTGFTNYANSVKGRFTISRDNAKNTVYLQMNS |
| (SEQ ID NO: 808) | LKPEDTAVYFCGATFLRSDGQHSIYGQGTQVTVSS |
| EPL164 | |
| Exemplary anti-target | QVQLQESGGGLVHTGGSLRLSCAASGDTFLRYAMGWFRQAPG |
| (EpCAM) | KEREFVAAITWNGGNTDYAGSLKGRFTISRDNTKNTVYLQMN |
| (SEQ ID NO: 809) EPL31 | SLKPEDTAVYYCAADLTFGLASSHYQYDYWGQGTQVTVSS |
| Exemplary anti-target | QVQLQESGGGLVQAGGSLRLSCAASGDTFLRYAMGWFRQAPG |
| (EpCAM) | KEREFVAAITWNGGNTDYAGSLKGRFTISRDNTKNTVYLQMN |
| (SEQ ID NO: 810) EPL55 | SLKPEDTAVYYCAADLTFGLASSHYQYDYWGQGTQVTVSS |
| Exemplary anti-target | QVQLQESGGGLVHTGGSLRLSCAFSGDTFLRYAMGWFRQAPG |
| (EpCAM) | KEREFVAAITWNGGNTDYADSLKGRFTISRDNTKNTVYLQMN |
| (SEQ ID NO: 811) EPL57 | SLRPEDTAVYYCAADLTFGLASSHYQYDYWGQGTQVTVSS |
| Exemplary anti-target | QVQLQESGGGLVQPGGSLRLSCAASGDTFLRYAMGWFRQAPG |
| (EpCAM) | KEREFVAAITWNGGNTDYAGSLKGRFTISRDNTKNTVYLQMN |
| (SEQ ID NO: 812) | SLKPEDTAVYYCAADLTFGLASSHYQYDYWGQGTQVTVSS |
| EPL136 | |
| Exemplary anti-target | QVQLQESGGGSVLAGGSLRLSCAASGFTFSSYYMSWVRQAPG |
| (EpCAM) | KGLEWVSGIHYTGDWTNYADSVKGRFTISRDNAKNELYLEMN |
| (SEQ ID NO: 813) EPL15 | NLKPEDTAVYYCARGSDKGQGTQVTVSS |
| Exemplary anti-target | QVQLQESGGGSVQAGGSLRLSCAASGFTFSSYYMSWVRQAPG |
| (EpCAM) | KGLEWVSGIHYTGDWTNYADSVKGRFTISRDNAKNELYLEMN |
| (SEQ ID NO: 814) EPL34 | NLKPEDTAVYYCARGSDKGQGTQVTVSS |
| Exemplary anti-target | QVQLQESGGGLVQPGGSLRLSCAASGFTFSDWAMSWVRQAPG |
| (EpCAM) | KGLEWVSGIHYGDHTTHYADFVKGRFTISRDDAKNTLYLQMN |
| (SEQ ID NO: 815) EPL86 | SLKPEDTAVYYCARGSTKGQGTQVTVSS |
| Exemplary anti-target | QVQLQESGGGLVQPGGSLRLSCAASGFTFSDWAMSWVRQAPG |
| (EpCAM) | KGLEWVSSIHYGDHTTHYADFVKGRFTISRDDAKNTLYLQMN |
| (SEQ ID NO: 816) | SLKPEDTAVYYCEKGTTRGQGTQVTVSS |
| EPL153 | |
| Exemplary anti-target | QVQLQESGGGLVQAGGSLKLSCAASGNVFRAATMAWYRQAPE |
| (EpCAM) | KQREMVATIASGGTTNYADFVKGRFTISRDNAKNTVYLQMNT |
| (SEQ ID NO: 817) EPL20 | LKPEDTAVYYCNAGYLTSLGPKNYWGQGTQVTVSS |
| Exemplary anti-target | QVQLQESGGGLVQPGGSLRLSCAASGNVFRAATMAWYRQAPE |
| (EpCAM) | KXREMVATIASGGTTNYADFVKGRFTISRDNAKNTVYLQMNT |
| (SEQ ID NO: 818) EPL70 | LKPEDTAVYYCNAGYLTSLGPKNYWGQGTQVTVSS |
| Exemplary anti-target | QVQLQESGGGLVQAGGSLRLSCAASGNVFRAATMAWYRQVPE |
| (EpCAM) | KQREMVATIASGGTTNYADFVKGRFTISRDNAKNTVYLQMNT |
| (SEQ ID NO: 819) | LKPEDTAVYYCNALYLTSLGPKSYWGQGTQVTVSS |
| EPL125 | |
| Exemplary anti-target | QVQLQESGGGLVQPGGSLRLSCAASGFAFGNHWMYWYRQAPG |
| (EpCAM) | RGRELVASISSGGSTNYVDSVKGRFTISRDNARNTVYLQMYS |
| (SEQ ID NO: 820) EPL13 | LKPEDTAVYYCGTSDNWGQGTQVTVSS |
| Exemplary anti-target | QVQLQESGGGLVQAGGSLRLSCAASGFAFGNHWMYWYRQAPG |
| (EpCAM) | RGRELVASISSGGSTNYVDSVKGRFTISRDNARNTVYLQMYS |
| (SEQ ID NO: 821) | LRPEDTAVYYCGTSDNWGQGTQVTVSS |
| EPL129 | |
| Exemplary anti-target | QVQLQESGGGLVQAGGSLRLSCAASGFAFGNHWMYWYRQAPG |
| (EpCAM) | RGRELVASISSGGSTNYVDSVKGRFTISRDNARNTVYLQMYS |
| (SEQ ID NO: 822) | LKPEDTAVYYCGTSDNWGQGTQVTVSS |
| EPL159 | |
| Exemplary anti-target | QVQLQESGGGLVQAGGSLRLSCAASGFIFRAASISWYRQSPG |
| (EpCAM) | NERELVATINSGGFTNYADSVLGRFTISRDNAKNTGYLQMNS |
| (SEQ ID NO: 823) | LKPEDTAVYFCAATFLRSDGQPPIWGQGTQVTVSS |
| EPL120 | |
| Exemplary anti-target | QVQLQESGGGLVQAGGSLRLSCAASGFIFRAASMGWYRQSPG |
| (EpCAM) | NERELVATINSGGFTNYADSVKGRFTISRDNAKNTGYLQMNS |
| (SEQ ID NO: 824) | LKPEDTAVYFCAATFLRSDGQPPIWGQGTQVTVSS |
| EPL126 | |
| Exemplary anti-target | QVQLQESGGGLVQAGGSLRLSCAASEYILSMYRMAWYRQAPG |
| (EpCAM) | KVRELVADMSSGGTTNYADFVKGRFTISRDNDRNTVYLQMNR |
| (SEQ ID NO: 825) EPL60 | LQPEDTAAYYCNVAGRTGPPSYDAFNNWGQGTQVTVSS |
| Exemplary anti-target | QVQLQESGGGLVQPGASLRVSCAASEYILSMYRMAWYRQAPG |
| (EpCAM) | KVRELVADMSSGGTTNYADFVKGRFTISRDNDRNTVYLQMNR |
| (SEQ ID NO: 826) | LQPEDTAAYYCNVAGRTGPPSYDAFNNWGQGTQVTVSS |
| EPL156 | |
| Exemplary anti-target | QVQLQESGGGLVQPGGSLRLSCAASESISSFIAVGWYRQAPG |
| (EpCAM) | KERELVAGINRSGFTYYTDSVKGRESISRDNAKNTVLLQMTS |
| (SEQ ID NO: 827) EPL2 | LKPEDTAVYYCNAGGLYFSNAYTQGDYWGQGTQVTVSS |
| Exemplary anti-target | QVQLQESGGGLVQTGGSLRLSCAASESISSFIAVGWYRQAPG |
| (EpCAM) | KERELVAGINRSGFTYYTDSVKGRESISRDNAKNTVLLQMTS |
| (SEQ ID NO: 828) EPL43 | LKPEDTAVYYCNAGGLYFSNAYTQGDYWGQGTQVTVSS |
| Exemplary anti-target | QVQLQESGGGLVQAGGSLRLSCAASGSVFRANVMGWYRQAPG |
| (EpCAM) | KQHELVARIDPGGTTTYADPVKGRFTISRDNAKKTVYLQMNS |
| (SEQ ID NO: 829) EPL10 | LKPDDTAVYYCNAIILLSGGPKDYWGQGTQVTVSS |
| Exemplary anti-target | QVQLQESGGGLVQAGGSLRLSCAPSGRTSSIFGMGWFRQAPG |
| (EpCAM) | KEREFVASINWSGGSTSYADSVKGRFTISRDNAKNEMYLQMN |
| (SEQ ID NO: 830) EPL49 | SLKFEDTAVYVCAAAVLTNKPSWNFWGQGTQVTVSS |
| Exemplary anti-target | QVQLQESGGGLVQAGGSLRLSCAASGPIFSDTIRTMGWYRQA |
| (EpCAM) | AGKQRELVATIASFPSRTNYVDSVKGRFTISRDIAKNTVYLQ |
| (SEQ ID NO: 831) EPL58 | MDSLKPEDTAVYYCNVDLASIPTKTYWGQGTQVTVSS |
| Exemplary anti-target | QVQLQESGGGLVQAGGSLRLSCAASGSIFGINAMGWYRQAPG |
| (EpCAM) | KQRESVAFITIGGNTNYLDSVKGRFTISRDNAKNTVYLQMNG |
| (SEQ ID NO: 832) EPL74 | LKPEDTAVYYCNTNPPLILTAGGLYWGQGTQVTVSS |
| Exemplary anti-target | QVQLQESGGGLVQPGGSLRLSCATSANRENINVMGWYRQAPG |
| (EpCAM) | QQRELVATINIGGSTDYADSVKGRFTISRDNAKNTVYLQLSD |
| (SEQ ID NO: 833) EPL78 | LKPEDTAVYYCNVKLRVSGPTGPNVYWGQGTQVTVSS |
| Exemplary anti-target | QVQLQESGGGLVQAGGSLKLSCTASGTILSTMAWYRQAPGKQ |
| (EpCAM) | RELVATISRGGTTNYSDSVKGRFAISRDSTKNTVYLQMNSLK |
| (SEQ ID NO: 834) EPL82 | PEDTAVYYCNTPLTDYGMGYNWGQGTQVTVSS |
| Exemplary anti-target | QVQLQESGGGLVQAGGSLRLSCAVSGSIFSLNTLAWYRQAPG |
| (EpCAM) | RQRDLIARITGGGTTVYADSVKGRFTISRDNAKNTVYLQMNS |
| (SEQ ID NO: 835) EPL83 | LKPEDTAVYYCNLMVRHPSGSTYEYWGQGTQVTVSS |
| Exemplary anti-target | QVQLQESGGGLVQAGGSLRLSCAASGIIFRGTTMGWFRQAPG |
| (EpCAM) | KQRESVASISPLGTTSYSGSVEGRFTVSRDNAKNTLFLQMNS |
| (SEQ ID NO: 836) EPL97 | LKSEDTAVYYCNAIQVTNVGPRVYWGQGTQVTVSS |
| Exemplary anti-target | QVQLQESGGGLVQPGGSLRLSCASSGFTLDDYTIGWFRQAPG |
| (EpCAM) | KEREGVSCISRRDDSTYYADSVKGRFTISRDNAKNTVDLQMI |
| (SEQ ID NO: 837) | SLRPEDTAVYYCAATPRSYTLRCLGKFDFQGQGTQVTVSS |
| EPL109 | |
| Exemplary anti-target | QVQLQESGGGLVQAGGSLRLSCAASGNIVRMTNMAWYRQAPG |
| (EpCAM) | KQREFVATISAGGSTTYVDSVKDRFTISRDNTKNTVYLQMNY |
| (SEQ ID NO: 838) | LKPEDTAVYYCATGSILTNRGAIPGSWGHGTQVTVSS |
| EPL117 | |
| Exemplary anti-target | QVQLQESGGGLVQAGGSLRLSCAAPGFAFNDHAILWFRQAPG |
| (EpCAM) | KEREGVSEICRDGTTYYTDSVKGRFTISSDNAKNTVYLQMNS |
| (SEQ ID NO: 839) | VKTDDTAVYYCAVDRRRYYCSGNRAFSSDYYYWGQGTQVTVS |
| EPL127 | S |
| Exemplary anti-target | QVQLQESGGGLVQAGGSLRLSCVHSGSIFRASTMAWYRQAPG |
| (EpCAM) | KQRELVAQIMSGGGTNYAGSVKGRFTISRDNANNTVYLQMNS |
| (SEQ ID NO: 840) | LKPEDTAVYYCNAAQITSWGPKVYWGQGTQVTVSS |
| EPL152 | |
| Exemplary anti-target | QVQLQESGGGLVQPGGSLRLSCAASGRINSINTMGWYRQAPG |
| (EpCAM) | NQRELVAEITRGGTTNYADSVQGRYAISRDNAKNLVYLQMNS |
| (SEQ ID NO: 841) | LKPEDTDVYYCNAQTFPTFSRPTGLDYWGQGTQVTVSS |
| EPL189 | |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 842) | SGVQPEDEAEYYCTLWYSNRWVFGGGTKLTVLGGGGSGGGGS |
| EPL10 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGSVF |
| | RANVMGWYRQAPGKQHELVARIDPGGTTTYADPVKGRFTISR |
| | DNAKKTVYLQMNSLKPDDTAVYYCNAIILLSGGPKDYWGQGT |
| | QVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 843) | SGVQPEDEAEYYCTLWYSNRWVFGGGTKLTVLGGGGSGGGGS |
| EPL109 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQPGGSLRLSCASSGFTL |
| | DDYTIGWFRQAPGKEREGVSCISRRDDSTYYADSVKGRFTIS |
| | RDNAKNTVDLQMISLRPEDTAVYYCAATPRSYTLRCLGKFDF |
| | QGQGTQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 844) | SGVQPEDEAEYYCTLWYSNRWVFGGGTKLTVLGGGGSGGGGS |
| EPL117 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGNIV |
| | RMTNMAWYRQAPGKQREFVATISAGGSTTYVDSVKDRFTISR |
| | DNTKNTVYLQMNYLKPEDTAVYYCATGSILTNRGAIPGSWGH |
| | GTQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 845) | SGVQPEDEAEYYCTLWYSNRWVFGGGTKLTVLGGGGSGGGGS |
| EPL120 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGFIF |
| | RAASISWYRQSPGNERELVATINSGGFTNYADSVLGRFTISR |
| | DNAKNTGYLQMNSLKPEDTAVYFCAATFLRSDGQPPIWGQGT |
| | QVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 846) | SGVQPEDEAEYYCTLWYSNRWVFGGGTKLTVLGGGGSGGGGS |
| EPL125 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGNVF |
| | RAATMAWYRQVPEKQREMVATIASGGTTNYADFVKGRFTISR |
| | DNAKNTVYLQMNTLKPEDTAVYYCNALYLTSLGPKSYWGQGT |
| | QVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 847) | SGVQPEDEAEYYCTLWYSNRWVFGGGTKLTVLGGGGSGGGGS |
| EPL127 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAAPGFAF |
| | NDHAILWFRQAPGKEREGVSEICRDGTTYYTDSVKGRFTISS |
| | DNAKNTVYLQMNSVKTDDTAVYYCAVDRRRYYCSGNRAFSSD |
| | YYYWGQGTQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 848) | SGVQPEDEAEYYCTLWYSNRWVFGGGTKLTVLGGGGSGGGGS |
| EPL13 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQPGGSLRLSCAASGFAF |
| | GNHWMYWYRQAPGRGRELVASISSGGSTNYVDSVKGRFTISR |
| | DNARNTVYLQMYSLKPEDTAVYYCGTSDNWGQGTQVTVSSHH |
| | HHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 849) | SGVQPEDEAEYYCTLWYSNRWVFGGGTKLTVLGGGGSGGGGS |
| EPL136 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQPGGSLRLSCAASGDTF |
| | LRYAMGWFRQAPGKEREFVAAITWNGGNTDYAGSLKGRFTIS |
| | RDNTKNTVYLQMNSLKPEDTAVYYCAADLTFGLASSHYQYDY |
| | WGQGTQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 850) | SGVQPEDEAEYYCTLWYSNRWVFGGGTKLTVLGGGGSGGGGS |
| EPL138 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGFIF |
| | RAASMDWYRQFPGNERESIATISSGGFTNYADSVKGRFTISR |
| | DNAKNTVYLQMNSLKPEDTAVYFCGATFLRSDGHHTINGQGT |
| | QVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 851) | SGVQPEDEAEYYCTLWYSNRWVFGGGTKLTVLGGGGSGGGGS |
| EPL145 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGFIF |
| | RAASMGWFRQSPGNERELVATVSSGGFTNYADSVKGRFTISR |
| | DNAKNTVYLQMNSLKPEDTAVYFCGATFVRSDGHHTIYGQGT |
| | QVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 852) | SGVQPEDEAEYYCTLWYSNRWVFGGGTKLTVLGGGGSGGGGS |
| EPL152 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCVHSGSIF |
| | RASTMAWYRQAPGKQRELVAQIMSGGGTNYAGSVKGRFTISR |
| | DNANNTVYLQMNSLKPEDTAVYYCNAAQITSWGPKVYWGQGT |
| | QVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 853) | SGVQPEDEAEYYCTLWYSNRWVFGGGTKLTVLGGGGSGGGGS |
| EPL153 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQPGGSLRLSCAASGFTF |
| | SDWAMSWVRQAPGKGLEWVSSIHYGDHTTHYADFVKGRFTIS |
| | RDDAKNTLYLQMNSLKPEDTAVYYCEKGTTRGQGTQVTVSSH |
| | HHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 854) | SGVQPEDEAEYYCTLWYSNRWVFGGGTKLTVLGGGGSGGGGS |
| EPL156 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQPGASLRVSCAASEYIL |
| | SMYRMAWYRQAPGKVRELVADMSSGGTTNYADFVKGRFTISR |
| | DNDRNTVYLQMNRLQPEDTAAYYCNVAGRTGPPSYDAFNNWG |
| | QGTQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 855) | SGVQPEDEAEYYCTLWYSNRWVFGGGTKLTVLGGGGSGGGGS |
| EPL164 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGFIF |
| | RAASMDWYRQSPGTQPELVATISSTGFTNYANSVKGRFTISR |
| | DNAKNTVYLQMNSLKPEDTAVYFCGATFLRSDGQHSIYGQGT |
| | QVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 856) | SGVQPEDEAEYYCTLWYSNRWVFGGGTKLTVLGGGGSGGGGS |
| EPL189 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQPGGSLRLSCAASGRIN |
| | SINTMGWYRQAPGNQRELVAEITRGGTTNYADSVQGRYAISR |
| | DNAKNLVYLQMNSLKPEDTDVYYCNAQTFPTFSRPTGLDYWG |
| | QGTQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 857) | SGVQPEDEAEYYCTLWYSNRWVFGGGTKLTVLGGGGSGGGGS |
| EPL2 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQPGGSLRLSCAASESIS |
| | SFIAVGWYRQAPGKERELVAGINRSGFTYYTDSVKGRESISR |
| | DNAKNTVLLQMTSLKPEDTAVYYCNAGGLYFSNAYTQGDYWG |
| | QGTQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 858) | SGVQPEDEAEYYCTLWYSNRWVFGGGTKLTVLGGGGSGGGGS |
| EPL20 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLKLSCAASGNVF |
| | RAATMAWYRQAPEKQREMVATIASGGTTNYADFVKGRFTISR |
| | DNAKNTVYLQMNTLKPEDTAVYYCNAGYLTSLGPKNYWGQGT |
| | QVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 859) | SGVQPEDEAEYYCTLWYSNRWVFGGGTKLTVLGGGGSGGGGS |
| EPL34 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGSVQAGGSLRLSCAASGFTF |
| | SSYYMSWVRQAPGKGLEWVSGIHYTGDWTNYADSVKGRFTIS |
| | RDNAKNELYLEMNNLKPEDTAVYYCARGSDKGQGTQVTVSSH |
| | HHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 860) | SGVQPEDEAEYYCTLWYSNRWVFGGGTKLTVLGGGGSGGGGS |
| EPL49 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAPSGRTS |
| | SIFGMGWFRQAPGKEREFVASINWSGGSTSYADSVKGRFTIS |
| | RDNAKNEMYLQMNSLKFEDTAVYVCAAAVLTNKPSWNFWGQG |
| | TQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 861) | SGVQPEDEAEYYCTLWYSNRWVFGGGTKLTVLGGGGSGGGGS |
| EPL58 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGPIF |
| | SDTIRTMGWYRQAAGKQRELVATIASFPSRTNYVDSVKGRFT |
| | ISRDIAKNTVYLQMDSLKPEDTAVYYCNVDLASIPTKTYWGQ |
| | GTQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 862) | SGVQPEDEAEYYCTLWYSNRWVFGGGTKLTVLGGGGSGGGGS |
| EPL74 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGSIF |
| | GINAMGWYRQAPGKQRESVAFITIGGNTNYLDSVKGRFTISR |
| | DNAKNTVYLQMNGLKPEDTAVYYCNTNPPLILTAGGLYWGQG |
| | TQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 863) | SGVQPEDEAEYYCTLWYSNRWVFGGGTKLTVLGGGGSGGGGS |
| EPL78 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQPGGSLRLSCATSANRF |
| | NINVMGWYRQAPGQQRELVATINIGGSTDYADSVKGRFTISR |
| | DNAKNTVYLQLSDLKPEDTAVYYCNVKLRVSGPTGPNVYWGQ |
| | GTQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 864) | SGVQPEDEAEYYCTLWYSNRWVFGGGTKLTVLGGGGSGGGGS |
| EPL82 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLKLSCTASGTIL |
| | STMAWYRQAPGKQRELVATISRGGTTNYSDSVKGRFAISRDS |
| | TKNTVYLQMNSLKPEDTAVYYCNTPLTDYGMGYNWGQGTQVT |
| | VSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 865) | SGVQPEDEAEYYCTLWYSNRWVFGGGTKLTVLGGGGSGGGGS |
| EPL83 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAVSGSIF |
| | SLNTLAWYRQAPGRQRDLIARITGGGTTVYADSVKGRFTISR |
| | DNAKNTVYLQMNSLKPEDTAVYYCNLMVRHPSGSTYEYWGQG |
| | TQVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 866) | SGVQPEDEAEYYCTLWYSNRWVFGGGTKLTVLGGGGSGGGGS |
| EPL86 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQPGGSLRLSCAASGFTF |
| | SDWAMSWVRQAPGKGLEWVSGIHYGDHTTHYADFVKGRFTIS |
| | RDDAKNTLYLQMNSLKPEDTAVYYCARGSTKGQGTQVTVSSH |
| | HHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 867) | SGVQPEDEAEYYCTLWYSNRWVFGGGTKLTVLGGGGSGGGGS |
| EPL90 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGFIF |
| | RAASMAWYRQSPGNERELVASISSGAFTNYADSVKARFTISR |
| | DNAKNTVYLQMNSLKPEDTAVYFCGATFLRSDGHHTINGQGT |
| | QVTVSSHHHHHH* |
| Exemplary anti- | VVGGGGTQTVVTQEPSLTVSPGGTVTLTCASSTGAVTSGNYP |
| CD3/anti-EpCAM fusion | NWVQQKPGQAPRGLIGGTKFLVPGTPARFSGSLLGGKAALTL |
| protein (SEQ ID NO: 868) | SGVQPEDEAEYYCTLWYSNRWVFGGGTKLTVLGGGGSGGGGS |
| EPL97 | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSQVQLQESGGGLVQAGGSLRLSCAASGIIF |
| | RGTTMGWFRQAPGKQRESVASISPLGTTSYSGSVEGRFTVSR |
| | DNAKNTLFLQMNSLKSEDTAVYYCNAIQVTNVGPRVYWGQGT |
| | QVTVSSHHHHHH* |
| Exemplary anti- | GGGSQVQLQESGGGLVQAGGSLRLSCAASGSVFRANVMGWYR |
| EpCAM/anti-EpCAM fusion | QAPGKQHELVARIDPGGTTTYADPVKGRFTISRDNAKKTVYL |
| protein (SEQ ID NO: 869) | QMNSLKPDDTAVYYCNAIILLSGGPKDYWGQGTQVTVSSGGG |
| EPL10 | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary anti- | GGGSQVQLQESGGGLVQPGGSLRLSCASSGFTLDDYTIGWER |
| EpCAM/anti-EpCAM fusion | QAPGKEREGVSCISRRDDSTYYADSVKGRFTISRDNAKNTVD |
| protein (SEQ ID NO: 870) | LQMISLRPEDTAVYYCAATPRSYTLRCLGKFDFQGQGTQVTV |
| EPL109 | SSGGGGSGGGSEVQLVESGGGLVQPGGSLKLSCAASGFTENK |
| | YAINWVRQAPGKGLEWVARIRSKYNNYATYYADQVKDRETIS |
| | RDDSKNTAYLQMNNLKTEDTAVYYCVRHANFGNSYISYWAYW |
| | GQGTLVTVSSGGGGSGGGGSGGGGSQTVVTQEPSLTVSPGGT |
| | VTLTCASSTGAVTSGNYPNWVQQKPGQAPRGLIGGTKFLVPG |
| | TPARFSGSLLGGKAALTLSGVQPEDEAEYYCTLWYSNRWVFG |
| | GGTKLTVLHHHHHH* |
| Exemplary anti- | GGGSQVQLQESGGGLVQAGGSLRLSCAASGNIVRMTNMAWYR |
| EpCAM/anti-EpCAM fusion | QAPGKQREFVATISAGGSTTYVDSVKDRFTISRDNTKNTVYL |
| protein (SEQ ID NO: 871) | QMNYLKPEDTAVYYCATGSILTNRGAIPGSWGHGTQVTVSSG |
| EPL117 | GGGSGGGSEVQLVESGGGLVQPGGSLKLSCAASGFTFNKYAI |
| | NWVRQAPGKGLEWVARIRSKYNNYATYYADQVKDRFTISRDD |
| | SKNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGT |
| | KLTVLHHHHHH* |
| Exemplary anti- | GGGSQVQLQESGGGLVQAGGSLRLSCAASGFIFRAASISWYR |
| EpCAM/anti-EpCAM fusion | QSPGNERELVATINSGGFTNYADSVLGRFTISRDNAKNTGYL |
| protein (SEQ ID NO: 872) | QMNSLKPEDTAVYFCAATFLRSDGQPPIWGQGTQVTVSSGGG |
| EPL120 | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary anti- | GGGSQVQLQESGGGLVQAGGSLRLSCAASGNVFRAATMAWYR |
| EpCAM/anti-EpCAM fusion | QVPEKQREMVATIASGGTTNYADFVKGRFTISRDNAKNTVYL |
| protein (SEQ ID NO: 873) | QMNTLKPEDTAVYYCNALYLTSLGPKSYWGQGTQVTVSSGGG |
| EPL125 | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary anti- | GGGSQVQLQESGGGLVQAGGSLRLSCAAPGFAFNDHAILWER |
| EpCAM/anti-EpCAM fusion | QAPGKEREGVSEICRDGTTYYTDSVKGRFTISSDNAKNTVYL |
| protein (SEQ ID NO: 874) | QMNSVKTDDTAVYYCAVDRRRYYCSGNRAFSSDYYYWGQGTQ |
| EPL127 | VTVSSGGGGSGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGSGGGGSQTVVTQEPSLTVSP |
| | GGTVTLTCASSTGAVTSGNYPNWVQQKPGQAPRGLIGGTKFL |
| | VPGTPARFSGSLLGGKAALTLSGVQPEDEAEYYCTLWYSNRW |
| | VFGGGTKLTVLHHHHHH* |
| Exemplary anti- | GGGSQVQLQESGGGLVQPGGSLRLSCAASGFAFGNHWMYWYR |
| EpCAM/anti-EpCAM fusion | QAPGRGRELVASISSGGSTNYVDSVKGRFTISRDNARNTVYL |
| protein (SEQ ID NO: 875) | QMYSLKPEDTAVYYCGTSDNWGQGTQVTVSSGGGGSGGGSEV |
| EPL13 | QLVESGGGLVQPGGSLKLSCAASGFTFNKYAINWVRQAPGKG |
| | LEWVARIRSKYNNYATYYADQVKDRFTISRDDSKNTAYLQMN |
| | NLKTEDTAVYYCVRHANFGNSYISYWAYWGQGTLVTVSSGGG |
| | GSGGGGSGGGGSQTVVTQEPSLTVSPGGTVTLTCASSTGAVT |
| | SGNYPNWVQQKPGQAPRGLIGGTKFLVPGTPARFSGSLLGGK |
| | AALTLSGVQPEDEAEYYCTLWYSNRWVFGGGTKLTVLHHHHH |
| | H* |
| Exemplary anti- | GGGSQVQLQESGGGLVQPGGSLRLSCAASGDTFLRYAMGWFR |
| EpCAM/anti-EpCAM fusion | QAPGKEREFVAAITWNGGNTDYAGSLKGRFTISRDNTKNTVY |
| protein (SEQ ID NO: 876) | LQMNSLKPEDTAVYYCAADLTFGLASSHYQYDYWGQGTQVTV |
| EPL136 | SSGGGGSGGGSEVQLVESGGGLVQPGGSLKLSCAASGFTENK |
| | YAINWVRQAPGKGLEWVARIRSKYNNYATYYADQVKDRFTIS |
| | RDDSKNTAYLQMNNLKTEDTAVYYCVRHANFGNSYISYWAYW |
| | GQGTLVTVSSGGGGSGGGGSGGGGSQTVVTQEPSLTVSPGGT |
| | VTLTCASSTGAVTSGNYPNWVQQKPGQAPRGLIGGTKFLVPG |
| | TPARFSGSLLGGKAALTLSGVQPEDEAEYYCTLWYSNRWVFG |
| | GGTKLTVLHHHHHH* |
| Exemplary anti- | GGGSQVQLQESGGGLVQAGGSLRLSCAASGFIFRAASMDWYR |
| EpCAM/anti-EpCAM fusion | QFPGNERESIATISSGGFTNYADSVKGRFTISRDNAKNTVYL |
| protein (SEQ ID NO: 877) | QMNSLKPEDTAVYFCGATFLRSDGHHTINGQGTQVTVSSGGG |
| EPL138 | GSGGGSEVQLVESGGGLVQPGGSLKLSCAASGFTFNKYAINW |
| | VRQAPGKGLEWVARIRSKYNNYATYYADQVKDRETISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary anti- | GGGSQVQLQESGGGLVQAGGSLRLSCAASGFIFRAASMGWFR |
| EpCAM/anti-EpCAM fusion | QSPGNERELVATVSSGGFTNYADSVKGRFTISRDNAKNTVYL |
| protein (SEQ ID NO: 878) | QMNSLKPEDTAVYFCGATFVRSDGHHTIYGQGTQVTVSSGGG |
| EPL145 | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary anti- | GGGSQVQLQESGGGLVQAGGSLRLSCVHSGSIFRASTMAWYR |
| EpCAM/anti-EpCAM fusion | QAPGKQRELVAQIMSGGGTNYAGSVKGRFTISRDNANNTVYL |
| protein (SEQ ID NO: 879) | QMNSLKPEDTAVYYCNAAQITSWGPKVYWGQGTQVTVSSGGG |
| EPL152 | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary anti- | GGGSQVQLQESGGGLVQPGGSLRLSCAASGFTFSDWAMSWVR |
| EpCAM/anti-EpCAM fusion | QAPGKGLEWVSSIHYGDHTTHYADFVKGRFTISRDDAKNTLY |
| protein (SEQ ID NO: 880) | LQMNSLKPEDTAVYYCEKGTTRGQGTQVTVSSGGGGSGGGSE |
| EPL153 | VQLVESGGGLVQPGGSLKLSCAASGFTFNKYAINWVRQAPGK |
| | GLEWVARIRSKYNNYATYYADQVKDRFTISRDDSKNTAYLQM |
| | NNLKTEDTAVYYCVRHANFGNSYISYWAYWGQGTLVTVSSGG |
| | GGSGGGGSGGGGSQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVLHHHH |
| | HH* |
| Exemplary anti- | GGGSQVQLQESGGGLVQPGASLRVSCAASEYILSMYRMAWYR |
| EpCAM/anti-EpCAM fusion | QAPGKVRELVADMSSGGTTNYADFVKGRFTISRDNDRNTVYL |
| protein (SEQ ID NO: 881) | QMNRLQPEDTAAYYCNVAGRTGPPSYDAFNNWGQGTQVTVSS |
| EPL156 | GGGGSGGGSEVQLVESGGGLVQPGGSLKLSCAASGFTFNKYA |
| | INWVRQAPGKGLEWVARIRSKYNNYATYYADQVKDRFTISRD |
| | DSKNTAYLQMNNLKTEDTAVYYCVRHANFGNSYISYWAYWGQ |
| | GTLVTVSSGGGGSGGGGSGGGGSQTVVTQEPSLTVSPGGTVT |
| | LTCASSTGAVTSGNYPNWVQQKPGQAPRGLIGGTKFLVPGTP |
| | ARFSGSLLGGKAALTLSGVQPEDEAEYYCTLWYSNRWVFGGG |
| | TKLTVLHHHHHH* |
| Exemplary anti- | GGGSQVQLQESGGGLVQAGGSLRLSCAASGFIFRAASMDWYR |
| EpCAM/anti-EpCAM fusion | QSPGTQPELVATISSTGFTNYANSVKGRFTISRDNAKNTVYL |
| protein (SEQ ID NO: 882) | QMNSLKPEDTAVYFCGATFLRSDGQHSIYGQGTQVTVSSGGG |
| EPL164 | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary anti- | GGGSQVQLQESGGGLVQPGGSLRLSCAASGRINSINTMGWYR |
| EpCAM/anti-EpCAM fusion | QAPGNQRELVAEITRGGTTNYADSVQGRYAISRDNAKNLVYL |
| protein (SEQ ID NO: 883) | QMNSLKPEDTDVYYCNAQTFPTFSRPTGLDYWGQGTQVTVSS |
| EPL189 | GGGGSGGGSEVQLVESGGGLVQPGGSLKLSCAASGFTFNKYA |
| | INWVRQAPGKGLEWVARIRSKYNNYATYYADQVKDRFTISRD |
| | DSKNTAYLQMNNLKTEDTAVYYCVRHANFGNSYISYWAYWGQ |
| | GTLVTVSSGGGGSGGGGSGGGGSQTVVTQEPSLTVSPGGTVT |
| | LTCASSTGAVTSGNYPNWVQQKPGQAPRGLIGGTKFLVPGTP |
| | ARFSGSLLGGKAALTLSGVQPEDEAEYYCTLWYSNRWVFGGG |
| | TKLTVLHHHHHH* |
| Exemplary anti- | GGGSQVQLQESGGGLVQPGGSLRLSCAASESISSFIAVGWYR |
| EpCAM/anti-EpCAM fusion | QAPGKERELVAGINRSGFTYYTDSVKGRESISRDNAKNTVLL |
| protein (SEQ ID NO: 884) | QMTSLKPEDTAVYYCNAGGLYFSNAYTQGDYWGQGTQVTVSS |
| EPL2 | GGGGSGGGSEVQLVESGGGLVQPGGSLKLSCAASGFTFNKYA |
| | INWVRQAPGKGLEWVARIRSKYNNYATYYADQVKDRFTISRD |
| | DSKNTAYLQMNNLKTEDTAVYYCVRHANFGNSYISYWAYWGQ |
| | GTLVTVSSGGGGSGGGGSGGGGSQTVVTQEPSLTVSPGGTVT |
| | LTCASSTGAVTSGNYPNWVQQKPGQAPRGLIGGTKFLVPGTP |
| | ARFSGSLLGGKAALTLSGVQPEDEAEYYCTLWYSNRWVFGGG |
| | TKLTVLHHHHHH* |
| Exemplary anti- | GGGSQVQLQESGGGLVQAGGSLKLSCAASGNVFRAATMAWYR |
| EpCAM/anti-EpCAM fusion | QAPEKQREMVATIASGGTTNYADFVKGRFTISRDNAKNTVYL |
| protein (SEQ ID NO: 885) | QMNTLKPEDTAVYYCNAGYLTSLGPKNYWGQGTQVTVSSGGG |
| EPL20 | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary anti- | GGGSQVQLQESGGGSVQAGGSLRLSCAASGFTFSSYYMSWVR |
| EpCAM/anti-EpCAM fusion | QAPGKGLEWVSGIHYTGDWTNYADSVKGRFTISRDNAKNELY |
| protein (SEQ ID NO: 886) | LEMNNLKPEDTAVYYCARGSDKGQGTQVTVSSGGGGSGGGSE |
| EPL34 | VQLVESGGGLVQPGGSLKLSCAASGFTFNKYAINWVRQAPGK |
| | GLEWVARIRSKYNNYATYYADQVKDRFTISRDDSKNTAYLQM |
| | NNLKTEDTAVYYCVRHANFGNSYISYWAYWGQGTLVTVSSGG |
| | GGSGGGGSGGGGSQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVLHHHH |
| | HH* |
| Exemplary anti- | GGGSQVQLQESGGGLVQAGGSLRLSCAPSGRTSSIFGMGWFR |
| EpCAM/anti-EpCAM fusion | QAPGKEREFVASINWSGGSTSYADSVKGRFTISRDNAKNEMY |
| protein (SEQ ID NO: 887) | LQMNSLKFEDTAVYVCAAAVLTNKPSWNFWGQGTQVTVSSGG |
| EPL49 | GGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQGT |
| | LVTVSSGGGGSGGGGSGGGGSQTVVTQEPSLTVSPGGTVTLT |
| | CASSTGAVTSGNYPNWVQQKPGQAPRGLIGGTKFLVPGTPAR |
| | FSGSLLGGKAALTLSGVQPEDEAEYYCTLWYSNRWVFGGGTK |
| | LTVLHHHHHH* |
| Exemplary anti- | GGGSQVQLQESGGGLVQAGGSLRLSCAASGPIFSDTIRTMGW |
| EpCAM/anti-EpCAM fusion | YRQAAGKQRELVATIASFPSRTNYVDSVKGRFTISRDIAKNT |
| protein (SEQ ID NO: 888) | VYLQMDSLKPEDTAVYYCNVDLASIPTKTYWGQGTQVTVSSG |
| EPL58 | GGGSGGGSEVQLVESGGGLVQPGGSLKLSCAASGFTFNKYAI |
| | NWVRQAPGKGLEWVARIRSKYNNYATYYADQVKDRFTISRDD |
| | SKNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGT |
| | KLTVLHHHHHH* |
| Exemplary anti- | GGGSQVQLQESGGGLVQAGGSLRLSCAASGSIFGINAMGWYR |
| EpCAM/anti-EpCAM fusion | QAPGKQRESVAFITIGGNTNYLDSVKGRFTISRDNAKNTVYL |
| protein (SEQ ID NO: 889) | QMNGLKPEDTAVYYCNTNPPLILTAGGLYWGQGTQVTVSSGG |
| EPL74 | GGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQGT |
| | LVTVSSGGGGSGGGGSGGGGSQTVVTQEPSLTVSPGGTVTLT |
| | CASSTGAVTSGNYPNWVQQKPGQAPRGLIGGTKFLVPGTPAR |
| | FSGSLLGGKAALTLSGVQPEDEAEYYCTLWYSNRWVEGGGTK |
| | LTVLHHHHHH* |
| Exemplary anti- | GGGSQVQLQESGGGLVQPGGSLRLSCATSANRENINVMGWYR |
| EpCAM/anti-EpCAM fusion | QAPGQQRELVATINIGGSTDYADSVKGRFTISRDNAKNTVYL |
| protein (SEQ ID NO: 890) | QLSDLKPEDTAVYYCNVKLRVSGPTGPNVYWGQGTQVTVSSG |
| EPL78 | GGGSGGGSEVQLVESGGGLVQPGGSLKLSCAASGFTFNKYAI |
| | NWVRQAPGKGLEWVARIRSKYNNYATYYADQVKDRFTISRDD |
| | SKNTAYLQMNNLKTEDTAVYYCVRHANFGNSYISYWAYWGQG |
| | TLVTVSSGGGGSGGGGSGGGGSQTVVTQEPSLTVSPGGTVTL |
| | TCASSTGAVTSGNYPNWVQQKPGQAPRGLIGGTKFLVPGTPA |
| | RFSGSLLGGKAALTLSGVQPEDEAEYYCTLWYSNRWVFGGGT |
| | KLTVLHHHHHH* |
| Exemplary anti- | GGGSQVQLQESGGGLVQAGGSLKLSCTASGTILSTMAWYRQA |
| EpCAM/anti-EpCAM fusion | PGKQRELVATISRGGTTNYSDSVKGRFAISRDSTKNTVYLQM |
| protein (SEQ ID NO: 891) | NSLKPEDTAVYYCNTPLTDYGMGYNWGQGTQVTVSSGGGGSG |
| EPL82 | GGSEVQLVESGGGLVQPGGSLKLSCAASGFTFNKYAINWVRQ |
| | APGKGLEWVARIRSKYNNYATYYADQVKDRFTISRDDSKNTA |
| | YLQMNNLKTEDTAVYYCVRHANFGNSYISYWAYWGQGTLVTV |
| | SSGGGGSGGGGSGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | HHHHHH* |
| Exemplary anti- | GGGSQVQLQESGGGLVQAGGSLRLSCAVSGSIFSLNTLAWYR |
| EpCAM/anti-EpCAM fusion | QAPGRQRDLIARITGGGTTVYADSVKGRFTISRDNAKNTVYL |
| protein (SEQ ID NO: 892) | QMNSLKPEDTAVYYCNLMVRHPSGSTYEYWGQGTQVTVSSGG |
| EPL83 | GGSGGGSEVQLVESGGGLVQPGGSLKLSCAASGFTFNKYAIN |
| | WVRQAPGKGLEWVARIRSKYNNYATYYADQVKDRFTISRDDS |
| | KNTAYLQMNNLKTEDTAVYYCVRHANFGNSYISYWAYWGQGT |
| | LVTVSSGGGGSGGGGSGGGGSQTVVTQEPSLTVSPGGTVTLT |
| | CASSTGAVTSGNYPNWVQQKPGQAPRGLIGGTKFLVPGTPAR |
| | FSGSLLGGKAALTLSGVQPEDEAEYYCTLWYSNRWVFGGGTK |
| | LTVLHHHHHH* |
| Exemplary anti- | GGGSQVQLQESGGGLVQPGGSLRLSCAASGFTFSDWAMSWVR |
| EpCAM/anti-EpCAM fusion | QAPGKGLEWVSGIHYGDHTTHYADFVKGRFTISRDDAKNTLY |
| protein (SEQ ID NO: 893) | LQMNSLKPEDTAVYYCARGSTKGQGTQVTVSSGGGGSGGGSE |
| EPL86 | VQLVESGGGLVQPGGSLKLSCAASGFTFNKYAINWVRQAPGK |
| | GLEWVARIRSKYNNYATYYADQVKDRFTISRDDSKNTAYLQM |
| | NNLKTEDTAVYYCVRHANFGNSYISYWAYWGQGTLVTVSSGG |
| | GGSGGGGSGGGGSQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVLHHHH |
| | HH* |
| Exemplary anti- | GGGSQVQLQESGGGLVQAGGSLRLSCAASGFIFRAASMAWYR |
| EpCAM/anti-EpCAM fusion | QSPGNERELVASISSGAFTNYADSVKARFTISRDNAKNTVYL |
| protein (SEQ ID NO: 894) | QMNSLKPEDTAVYFCGATFLRSDGHHTINGQGTQVTVSSGGG |
| EPL90 | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary anti- | GGGSQVQLQESGGGLVQAGGSLRLSCAASGIIFRGTTMGWFR |
| EpCAM/anti-EpCAM fusion | QAPGKQRESVASISPLGTTSYSGSVEGRFTVSRDNAKNTLFL |
| protein (SEQ ID NO: 895) | QMNSLKSEDTAVYYCNAIQVTNVGPRVYWGQGTQVTVSSGGG |
| EPL97 | GSGGGSEVQLVESGGGLVQPGGSLKLSCAASGFTFNKYAINW |
| | VRQAPGKGLEWVARIRSKYNNYATYYADQVKDRFTISRDDSK |
| | NTAYLQMNNLKTEDTAVYYCVRHANFGNSYISYWAYWGQGTL |
| | VTVSSGGGGSGGGGSGGGGSQTVVTQEPSLTVSPGGTVTLTC |
| | ASSTGAVTSGNYPNWVQQKPGQAPRGLIGGTKFLVPGTPARF |
| | SGSLLGGKAALTLSGVQPEDEAEYYCTLWYSNRWVFGGGTKL |
| | TVLHHHHHH* |
| Exemplary humanized | EVQLVESGGGLVQPGGSLTLSCAASGFAFGNHWMYWYRQAPG |
| anti-EpCAM sequence | RGRELVASISSGGSTNYVDSVKGRFTISRDNAKNTLYLQMNS |
| (SEQ ID No. 896) | LRAEDTAVYYCGTSDNWGQGTLVTVSS |
| Exemplary humanized | EVQLLESGGGLVQPGGSLTLSCAASGFIFRAASMDWYRQFPG |
| anti-EpCAM sequence | NERESIATISSGGFTNYADSVKGRFTISRDNSKNTLYLQMNS |
| (SEQ ID No. 897) | LRAEDTAVYYCGATFLRSDGHHTINGQGTLVTVSS |
| Exemplary humanized | EVQLLESGGGLVQPGGSLTLSCAASGFIFRAASMAWYRQSPG |
| anti-EpCAM sequence | NERELVASISSGAFTNYADSVKARFTISRDNSKNTLYLQMNS |
| (SEQ ID No. 898) | LRAEDTAVYYCGATFLRSDGHHTINGQGTLVTVSS |
| Exemplary Humanized | VVGGGGTQTVVTQEPSLTVSPGGTVTLTCASSTGAVTSGNYP |
| Anti-CD3/Anti-EpCAM | NWVQQKPGQAPRGLIGGTKFLVPGTPARFSGSLLGGKAALTL |
| sequence name H13 (SEQ | SGVQPEDEAEYYCTLWYSNRWVFGGGTKLTVLGGGGSGGGGS |
| ID No. 899) | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSEVQLVESGGGLVQPGGSLTLSCAASGFAF |
| | GNHWMYWYRQAPGRGRELVASISSGGSTNYVDSVKGRFTISR |
| | DNAKNTLYLQMNSLRAEDTAVYYCGTSDNWGQGTLVTVSSHH |
| | HHHH* |
| Exemplary Humanized | VVGGGGTQTVVTQEPSLTVSPGGTVTLTCASSTGAVTSGNYP |
| Anti-CD3/Anti-EpCAM | NWVQQKPGQAPRGLIGGTKFLVPGTPARFSGSLLGGKAALTL |
| sequence name H138 (SEQ | SGVQPEDEAEYYCTLWYSNRWVFGGGTKLTVLGGGGSGGGGS |
| ID No. 900) | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSEVQLLESGGGLVQPGGSLTLSCAASGFIF |
| | RAASMDWYRQFPGNERESIATISSGGFTNYADSVKGRFTISR |
| | DNSKNTLYLQMNSLRAEDTAVYYCGATFLRSDGHHTINGQGT |
| | LVTVSSHHHHHH* |
| Exemplary Humanized | VVGGGGTQTVVTQEPSLTVSPGGTVTLTCASSTGAVTSGNYP |
| Anti-CD3/Anti-EpCAM | NWVQQKPGQAPRGLIGGTKFLVPGTPARFSGSLLGGKAALTL |
| sequence name H90 (SEQ | SGVQPEDEAEYYCTLWYSNRWVFGGGTKLTVLGGGGSGGGGS |
| ID No. 901) | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSEVQLLESGGGLVQPGGSLTLSCAASGFIF |
| | RAASMAWYRQSPGNERELVASISSGAFTNYADSVKARFTISR |
| | DNSKNTLYLQMNSLRAEDTAVYYCGATFLRSDGHHTINGQGT |
| | LVTVSSHHHHHH* |
| SEQ ID No. 902 | QDGNEEMGGITQ |
| SEQ ID No. 903 | LVQPGN |
| AB loop oligo WT | |
| SEQ ID No. 904 | DSVKGR |
| C′′D loop oligo WT | |
| SEQ ID No. 905 | SLRPED |
| EF loop oligo WT | |
| EpCAM H90 TriTAC | EVQLVESGGGLVQPGNSLRLSCAASGFTFSKFGMSWVRQAPG |
| C2854 | KGLEWVSSISGSGRDTLYADSVKGRFTISRDNAKTTLYLQMN |
| (SEQ ID No. 906) | SLRPEDTAVYYCTIGGSLSVSSQGTLVTVSSGGGGSGGGSQT |
| | VVTQEPSLTVSPGGTVTLTCASSTGAVTSGNYPNWVQQKPGQ |
| | APRGLIGGTKFLVPGTPARFSGSLLGGKAALTLSGVQPEDEA |
| | EYYCTLWYSNRWVFGGGTKLTVLGGGGSGGGGSGGGGSEVQL |
| | VESGGGLVQPGGSLKLSCAASGFTFNKYAINWVRQAPGKGLE |
| | WVARIRSKYNNYATYYADQVKDRFTISRDDSKNTAYLQMNNL |
| | KTEDTAVYYCVRHANFGNSYISYWAYWGQGTLVTVSSGGGGS |
| | GGGSEVQLLESGGGLVQPGGSLTLSCAASGFIFRAASMAWYR |
| | QSPGNERELVASISSGAFTNYADSVKARFTISRDNSKNTLYL |
| | QMNSLRAEDTAVYYCGATFLRSDGHHTINGQGTLVTVSSHHH |
| | HHH |
| EpCAM H90 ProTriTAC | QTVVTQEPSLTVSPGGTVTLTCASSTGAVTSGNYPNWVQQKP |
| (L040) C2704 | GQAPRGLIGGTKFLVPGTPARFSGSLLGGKAALTLSGVQPED |
| (SEQ ID No. 907) | EAEYYCTLWYSNRWVFGGGTKLTVLGGGGSGGGGSGGGGSEV |
| | QLVESGGGLVQPGGSLKLSCAASGFTFNKYAINWVRQAPGKG |
| | LEWVARIRSKYNNYATYYADQVKDRFTISRDDSKNTAYLQMN |
| | NLKTEDTAVYYCVRHANFGNSYISYWAYWGQGTLVTVSSGGG |
| | GSGGGSEVQLLESGGGLVQPGGSLTLSCAASGFIFRAASMAW |
| | YRQSPGNERELVASISSGAFTNYADSVKARFTISRDNSKNTL |
| | YLQMNSLRAEDTAVYYCGATFLRSDGHHTINGQGTLVTVSSH |
| | HHHHH |
| EpCAM H90 ProTriTAC | EVQLVESGGGLVQPGNSLRLSCAASGFTFSKFGMSWVRQGGG |
| (NCLV) C2302 | GGLDGNEEPGGLEWVSSISGSGRDTLYADSVKGRFTISRDNA |
| (SEQ ID No. 908) | KTTLYLQMNSLRPEDTAVYYCTIGGSLSVSSQGTLVTVSSGG |
| | GGSGGGGSGGVVGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTINGQGTLVTVSSHHHHHH |
| L043 Linker Protease | PQGSTARSAG |
| Sequence | |
| (SEQ ID NO: 909) | |
| L054 Linker Protease | VANLASTGRA |
| Sequence | |
| (SEQ ID NO: 910) | |
| L055 Linker Protease | PLGLSTGRA |
| Sequence | |
| (SEQ ID NO: 911) | |
| L056 Linker Protease | PQASTGRA |
| Sequence | |
| (SEQ ID NO: 912) | |
| L057 Linker Protease | PKPQASTGRA |
| Sequence | |
| (SEQ ID NO: 913) | |
| L058 Linker Protease | PTGLESTGRA |
| Sequence | |
| (SEQ ID NO: 914) | |
| L059 Linker Protease | PENFFSTGRA |
| Sequence | |
| (SEQ ID NO: 915) | |
| L060 Linker Protease | AATLKSTGRA |
| Sequence | |
| (SEQ ID NO: 916) | |
| L061 Linker Protease | PQDLLSTGRA |
| Sequence | |
| (SEQ ID NO: 917) | |
| L062 Linker Protease | PAGLSTGRA |
| Sequence | |
| (SEQ ID NO: 918) | |
| L063 Linker Protease | VLGLSTGRA |
| Sequence | |
| (SEQ ID NO: 919) | |
| L064 Linker Protease | VANLASTGA |
| Sequence | |
| (SEQ ID NO: 920) | |
| L065 Linker Protease | PLGLSTGA |
| Sequence | |
| (SEQ ID NO: 921) | |
| L066 Linker Protease | PQASTGA |
| Sequence | |
| (SEQ ID NO: 922) | |
| L067 Linker Protease | PKPQASTGA |
| Sequence | |
| (SEQ ID NO: 923) | |
| L068 Linker Protease | PTGLESTGA |
| Sequence | |
| (SEQ ID NO: 924) | |
| L069 Linker Protease | PENFFSTGA |
| Sequence | |
| (SEQ ID NO: 925) | |
| L070 Linker Protease | AATLKSTGA |
| Sequence | |
| (SEQ ID NO: 926) | |
| L071 Linker Protease | PQDLLSTGA |
| Sequence | |
| (SEQ ID NO: 927) | |
| L072 Linker Protease | PAGLSTGA |
| Sequence | |
| (SEQ ID NO: 928) | |
| L073 Linker Protease | VLGLSTGA |
| Sequence | |
| (SEQ ID NO: 929) | |
| L075 Linker Protease | RTRYEDGGS |
| Sequence | |
| (SEQ ID NO: 930) | |
| L076 Linker Protease | PQASTGGSG |
| Sequence | |
| (SEQ ID NO: 931) | |
| L040 Full Linker | GGGGPQASTGRSGGGGGGT |
| Sequence version 1 | |
| (SEQ ID NO: 932) | |
| L041 Full Linker | GGGGPQGSTGRAAGGGGGT |
| Sequence version 1 | |
| (SEQ ID NO: 933) | |
| L043 Full Linker | GGGGPQGSTARSAGGGGGT |
| Sequence version 1 | |
| (SEQ ID NO: 934) | |
| L045 Full Linker | GGGGPIPVQGRAHGGGGT |
| Sequence version 1 | |
| (SEQ ID NO: 935) | |
| L054 Full Linker | GGGGVANLASTGRAGGGGT |
| Sequence version 1 | |
| (SEQ ID NO: 936) | |
| L055 Full Linker | GGGGPLGLSTGRAGGGGT |
| Sequence version 1 | |
| (SEQ ID NO: 937) | |
| L056 Full Linker | GGGGPQASTGRAGGGT |
| Sequence version 1 | |
| (SEQ ID NO: 938) | |
| L057 Full Linker | GGGGPKPQASTGRAGGGT |
| Sequence version 1 | |
| (SEQ ID NO: 939) | |
| L058 Full Linker | GGGGPTGLESTGRAGGGT |
| Sequence version 1 | |
| (SEQ ID NO: 940) | |
| L059 Full Linker | GGGGPENFFSTGRAGGGT |
| Sequence version 1 | |
| (SEQ ID NO: 941) | |
| L060 Full Linker | GGGGAATLKSTGRAGGGT |
| Sequence version 1 | |
| (SEQ ID NO: 942) | |
| L061 Full Linker | GGGGPQDLLSTGRAGGGT |
| Sequence version 1 | |
| (SEQ ID NO: 943) | |
| L062 Full Linker | GGGGPAGLSTGRAGGGT |
| Sequence version 1 | |
| (SEQ ID NO: 944) | |
| L063 Full Linker | GGGGVLGLSTGRAGGGT |
| Sequence version 1 | |
| (SEQ ID NO: 945) | |
| L064 Full Linker | GGGGVANLASTGAGGGT |
| Sequence version 1 | |
| (SEQ ID NO: 946) | |
| L065 Full Linker | GGGGPLGLSTGAGGGT |
| Sequence version 1 | |
| (SEQ ID NO: 947) | |
| L066 Full Linker | GGGGPQASTGAGGGT |
| Sequence version 1 | |
| (SEQ ID NO: 948) | |
| L067 Full Linker | GGGGPKPQASTGAGGGT |
| Sequence version 1 | |
| (SEQ ID NO: 949) | |
| L068 Full Linker | GGGGPTGLESTGAGGGT |
| Sequence version 1 | |
| (SEQ ID NO: 950) | |
| L069 Full Linker | GGGGPENFFSTGAGGGT |
| Sequence version 1 | |
| (SEQ ID NO: 951) | |
| L070 Full Linker | GGGGAATLKSTGAGGGT |
| Sequence version 1 | |
| (SEQ ID NO: 952) | |
| L071 Full Linker | GGGGPQDLLSTGAGGGT |
| Sequence version 1 | |
| (SEQ ID NO: 953) | |
| L072 Full Linker | GGGGPAGLSTGAGGGT |
| Sequence version 1 | |
| (SEQ ID NO: 954) | |
| L073 Full Linker | GGGGVLGLSTGAGGGT |
| Sequence version 1 | |
| (SEQ ID NO: 955) | |
| L075 Full Linker | GGGGRTRYEDGGSGGGT |
| Sequence version 1 | |
| (SEQ ID NO: 956) | |
| L076 Full Linker | GGGGPQASTGGSGGGGT |
| Sequence version 1 | |
| (SEQ ID NO: 957) | |
| L001 Full Linker | GGGGKPLGLQARVVGGGGS |
| Sequence version 2 | |
| (SEQ ID NO: 958) | |
| L040 Full Linker | GGGGPQASTGRSGGGGGGS |
| Sequence version 2 | |
| (SEQ ID NO: 959) | |
| L041 Full Linker | GGGGPQGSTGRAAGGGGGS |
| Sequence version 2 | |
| (SEQ ID NO: 960) | |
| L043 Full Linker | GGGGPQGSTARSAGGGGGS |
| Sequence version 2 | |
| (SEQ ID NO: 961) | |
| L045 Full Linker | GGGGPIPVQGRAHGGGGS |
| Sequence version 2 | |
| (SEQ ID NO: 962) | |
| L054 Full Linker | GGGGVANLASTGRAGGGGS |
| Sequence version 2 | |
| (SEQ ID NO: 963) | |
| L055 Full Linker | GGGGPLGLSTGRAGGGGS |
| Sequence version 2 | |
| (SEQ ID NO: 964) | |
| L056 Full Linker | GGGGPQASTGRAGGGTS |
| Sequence version 2 | |
| (SEQ ID NO: 965) | |
| L057 Full Linker | GGGGPKPQASTGRAGGGS |
| Sequence version 2 | |
| (SEQ ID NO: 966) | |
| L058 Full Linker | GGGGPTGLESTGRAGGGS |
| Sequence version 2 | |
| (SEQ ID NO: 967) | |
| L059 Full Linker | GGGGPENFFSTGRAGGGS |
| Sequence version 2 | |
| (SEQ ID NO: 968) | |
| L060 Full Linker | GGGGAATLKSTGRAGGGS |
| Sequence version 2 | |
| (SEQ ID NO: 969) | |
| L061 Full Linker | GGGGPQDLLSTGRAGGGS |
| Sequence version 2 | |
| (SEQ ID NO: 970) | |
| L062 Full Linker | GGGGPAGLSTGRAGGGS |
| Sequence version 2 | |
| (SEQ ID NO: 971) | |
| L063 Full Linker | GGGGVLGLSTGRAGGGS |
| Sequence version 2 | |
| (SEQ ID NO: 972) | |
| L064 Full Linker | GGGGVANLASTGAGGGS |
| Sequence version 2 | |
| (SEQ ID NO: 973) | |
| L065 Full Linker | GGGGPLGLSTGAGGGS |
| Sequence version 2 | |
| (SEQ ID NO: 974) | |
| L066 Full Linker | GGGGPQASTGAGGGS |
| Sequence version 2 | |
| (SEQ ID NO: 975) | |
| L067 Full Linker | GGGGPKPQASTGAGGGS |
| Sequence version 2 | |
| (SEQ ID NO: 976) | |
| L068 Full Linker | GGGGPTGLESTGAGGGS |
| Sequence version 2 | |
| (SEQ ID NO: 977) | |
| L069 Full Linker | GGGGPENFFSTGAGGGS |
| Sequence version 2 | |
| (SEQ ID NO: 978) | |
| L070 Full Linker | GGGGAATLKSTGAGGGS |
| Sequence version 2 | |
| (SEQ ID NO: 979) | |
| L071 Full Linker | GGGGPQDLLSTGAGGGS |
| Sequence version 2 | |
| (SEQ ID NO: 980) | |
| L072 Full Linker | GGGGPAGLSTGAGGGS |
| Sequence version 2 | |
| (SEQ ID NO: 981) | |
| L073 Full Linker | GGGGVLGLSTGAGGGS |
| Sequence version 2 | |
| (SEQ ID NO: 982) | |
| L075 Full Linker | GGGGRTRYEDGGSGGGS |
| Sequence version 2 | |
| (SEQ ID NO: 983) | |
| L076 Full Linker | GGGGPQASTGGSGGGGS |
| Sequence version 2 | |
| (SEQ ID NO: 984) | |
| protease cleavage site | VANLA |
| (SEQ ID NO: 985) | |
| protease cleavage site | PLGL |
| (SEQ ID NO: 986) | |
| protease cleavage site | PQAS |
| (SEQ ID NO: 987) | |
| protease cleavage site | PKPQA |
| (SEQ ID NO: 988) | |
| protease cleavage site | PTGLE |
| (SEQ ID NO: 989) | |
| protease cleavage site | PENFF |
| (SEQ ID NO: 990) | |
| protease cleavage site | AATLK |
| (SEQ ID NO: 991) | |
| protease cleavage site | PQDLL |
| (SEQ ID NO: 992) | |
| protease cleavage site | PAGL |
| (SEQ ID NO: 993) | |
| protease cleavage site | VLGL |
| (SEQ ID NO: 994) | |
| protease cleavage site | RTRYED |
| (SEQ ID NO: 995) | |
| protease cleavage site | KPLGL |
| (SEQ ID NO: 996) | |
| Anti-EpCAM H138.2 | EVQLLESGGGLVQPGGSLTLSCAASGFIFRAASMDWYRQFPG |
| (SEQ ID NO: 997) | NERESIATISSGGFTNYADSVKGRFTISRDNSKNTLYLQMNS |
| | LRAEDTAVYYCGATFLRSDGHHTIWGQGTLVTVSS |
| Anti-EpCAM H90.2 | EVQLLESGGGLVQPGGSLTLSCAASGFIFRAASMAWYRQSPG |
| (SEQ ID NO: 998) | NERELVASISSGAFTNYADSVKARFTISRDNSKNTLYLQMNS |
| | LRAEDTAVYYCGATFLRSDGHHTIWGQGTLVTVSS |
| AxCT mask27 EpCAM H90.2 | EVQLVESGGGLVQPGNSLRLSCAASGFTFSKFGMSWVRQGGG |
| L040 version 1 | GGLDGNEEPGGLEWVSSISGSGRDTLYADSVKGRFTISRDNA |
| (SEQ ID NO: 999) | KTTLYLQMNSLRPEDTAVYYCTIGGSLSVSSQGTLVTVSSGG |
| | GGPQASTGRSGGGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTIWGQGTLVTVSSHHHHHH |
| AxCT mask27 EpCAM H90.2 | EVQLVESGGGLVQPGNSLRLSCAASGFTFSKFGMSWVRQGGG |
| L041 version 1 | GGLDGNEEPGGLEWVSSISGSGRDTLYADSVKGRFTISRDNA |
| (SEQ ID NO: 1000) | KTTLYLQMNSLRPEDTAVYYCTIGGSLSVSSQGTLVTVSSGG |
| | GGPQGSTGRAAGGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TENKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTIWGQGTLVTVSSHHHHHH |
| AxCT mask27 EpCAM H90.2 | EVQLVESGGGLVQPGNSLRLSCAASGFTFSKFGMSWVRQGGG |
| L043 version 1 | GGLDGNEEPGGLEWVSSISGSGRDTLYADSVKGRFTISRDNA |
| (SEQ ID NO: 1001) | KTTLYLQMNSLRPEDTAVYYCTIGGSLSVSSQGTLVTVSSGG |
| | GGPQGSTARSAGGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TENKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTIWGQGTLVTVSSHHHHHH |
| AxCT mask27 EpCAM H90.2 | EVQLVESGGGLVQPGNSLRLSCAASGFTFSKFGMSWVRQGGG |
| L076 version 1 | GGLDGNEEPGGLEWVSSISGSGRDTLYADSVKGRFTISRDNA |
| (SEQ ID NO: 1002) | KTTLYLQMNSLRPEDTAVYYCTIGGSLSVSSQGTLVTVSSGG |
| | GGPQASTGGSGGGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TENKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTIWGQGTLVTVSSHHHHHH |
| AxCT mask27 EpCAM H90.2 | EVQLVESGGGLVQPGNSLRLSCAASGFTFSKFGMSWVRQGGG |
| L059 version 1 | GGLDGNEEPGGLEWVSSISGSGRDTLYADSVKGRFTISRDNA |
| (SEQ ID NO: 1003) | KTTLYLQMNSLRPEDTAVYYCTIGGSLSVSSQGTLVTVSSGG |
| | GGPENFFSTGRAGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGSEVQLLESGGGLVQPGGSLTL |
| | SCAASGFIFRAASMAWYRQSPGNERELVASISSGAFTNYADS |
| | VKARFTISRDNSKNTLYLQMNSLRAEDTAVYYCGATFLRSDG |
| | HHTIWGQGTLVTVSSHHHHHH |
| AxCT mask27 EpCAM H90.2 | EVQLVESGGGLVQPGNSLRLSCAASGFTFSKFGMSWVRQGGG |
| L060 version 1 | GGLDGNEEPGGLEWVSSISGSGRDTLYADSVKGRFTISRDNA |
| (SEQ ID NO: 1004) | KTTLYLQMNSLRPEDTAVYYCTIGGSLSVSSQGTLVTVSSGG |
| | GGAATLKSTGRAGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGSEVQLLESGGGLVQPGGSLTL |
| | SCAASGFIFRAASMAWYRQSPGNERELVASISSGAFTNYADS |
| | VKARFTISRDNSKNTLYLQMNSLRAEDTAVYYCGATFLRSDG |
| | HHTIWGQGTLVTVSSHHHHHH |
| AxCT mask27 EpCAM H90.2 | EVQLVESGGGLVQPGNSLRLSCAASGFTFSKFGMSWVRQGGG |
| L061 version 1 | GGLDGNEEPGGLEWVSSISGSGRDTLYADSVKGRFTISRDNA |
| (SEQ ID NO: 1005) | KTTLYLQMNSLRPEDTAVYYCTIGGSLSVSSQGTLVTVSSGG |
| | GGPQDLLSTGRAGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGSEVQLLESGGGLVQPGGSLTL |
| | SCAASGFIFRAASMAWYRQSPGNERELVASISSGAFTNYADS |
| | VKARFTISRDNSKNTLYLQMNSLRAEDTAVYYCGATFLRSDG |
| | HHTIWGQGTLVTVSSHHHHHH |
| AxCT mask27 EpCAM H90.2 | EVQLVESGGGLVQPGNSLRLSCAASGFTFSKFGMSWVRQGGG |
| L063 version 1 | GGLDGNEEPGGLEWVSSISGSGRDTLYADSVKGRFTISRDNA |
| (SEQ ID NO: 1006) | KTTLYLQMNSLRPEDTAVYYCTIGGSLSVSSQGTLVTVSSGG |
| | GGVLGLSTGRAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGSEVQLLESGGGLVQPGGSLTLS |
| | CAASGFIFRAASMAWYRQSPGNERELVASISSGAFTNYADSV |
| | KARFTISRDNSKNTLYLQMNSLRAEDTAVYYCGATFLRSDGH |
| | HTIWGQGTLVTVSSHHHHHH |
| AxCT mask27 EpCAM H90.2 | EVQLVESGGGLVQPGNSLRLSCAASGFTFSKFGMSWVRQGGG |
| L064 version 1 | GGLDGNEEPGGLEWVSSISGSGRDTLYADSVKGRFTISRDNA |
| (SEQ ID NO: 1007) | KTTLYLQMNSLRPEDTAVYYCTIGGSLSVSSQGTLVTVSSGG |
| | GGVANLASTGAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGSEVQLLESGGGLVQPGGSLTLS |
| | CAASGFIFRAASMAWYRQSPGNERELVASISSGAFTNYADSV |
| | KARFTISRDNSKNTLYLQMNSLRAEDTAVYYCGATFLRSDGH |
| | HTIWGQGTLVTVSSHHHHHH |
| AxCT mask27 EpCAM H90.2 | EVQLVESGGGLVQPGNSLRLSCAASGFTFSKFGMSWVRQGGG |
| L073 version 1 | GGLDGNEEPGGLEWVSSISGSGRDTLYADSVKGRFTISRDNA |
| (SEQ ID NO: 1008) | KTTLYLQMNSLRPEDTAVYYCTIGGSLSVSSQGTLVTVSSGG |
| | GGVLGLSTGAGGGTQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGSEVQLLESGGGLVQPGGSLTLSC |
| | AASGFIFRAASMAWYRQSPGNERELVASISSGAFTNYADSVK |
| | ARFTISRDNSKNTLYLQMNSLRAEDTAVYYCGATFLRSDGHH |
| | TIWGQGTLVTVSSHHHHHH |
| AxCT mask27 EpCAM H90.2 | EVQLVESGGGLVQPGNSLRLSCAASGFTFSKFGMSWVRQGGG |
| L075 version 1 | GGLDGNEEPGGLEWVSSISGSGRDTLYADSVKGRFTISRDNA |
| (SEQ ID NO: 1009) | KTTLYLQMNSLRPEDTAVYYCTIGGSLSVSSQGTLVTVSSGG |
| | GGRTRYEDGGSGGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGSEVQLLESGGGLVQPGGSLTL |
| | SCAASGFIFRAASMAWYRQSPGNERELVASISSGAFTNYADS |
| | VKARFTISRDNSKNTLYLQMNSLRAEDTAVYYCGATFLRSDG |
| | HHTIWGQGTLVTVSSHHHHHH |
| AxCT mask27 EpCAM H90 | EVQLVESGGGLVQPGNSLRLSCAASGFTFSKFGMSWVRQGGG |
| L001 version 1 | GGLDGNEEPGGLEWVSSISGSGRDTLYADSVKGRFTISRDNA |
| (SEQ ID NO: 1010) | KTTLYLQMNSLRPEDTAVYYCTIGGSLSVSSQGTLVTVSSGG |
| | GGKPLGLQARVVGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TENKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTINGQGTLVTVSSHHHHHH |
| AxCT mask27 EpCAM H90.2 | EVQLVESGGGLVQPGNSLRLSCAASGFTFSKFGMSWVRQGGG |
| NCLV | GGLDGNEEPGGLEWVSSISGSGRDTLYADSVKGRFTISRDNA |
| (SEQ ID NO: 1011) | KTTLYLQMNSLRPEDTAVYYCTIGGSLSVSSQGTLVTVSSGG |
| | GGSGGGGSGGVVGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTIWGQGTLVTVSSHHHHHH |
| stubCT EpCAM H90.2 | VVGGGGTQTVVTQEPSLTVSPGGTVTLTCASSTGAVTSGNYP |
| (SEQ ID NO: 1012) | NWVQQKPGQAPRGLIGGTKFLVPGTPARFSGSLLGGKAALTL |
| | SGVQPEDEAEYYCTLWYSNRWVFGGGTKLTVLGGGGSGGGGS |
| | GGGGSEVQLVESGGGLVQPGGSLKLSCAASGFTFNKYAINWV |
| | RQAPGKGLEWVARIRSKYNNYATYYADQVKDRFTISRDDSKN |
| | TAYLQMNNLKTEDTAVYYCVRHANFGNSYISYWAYWGQGTLV |
| | TVSSGGGGSGGGSEVQLLESGGGLVQPGGSLTLSCAASGFIF |
| | RAASMAWYRQSPGNERELVASISSGAFTNYADSVKARFTISR |
| | DNSKNTLYLQMNSLRAEDTAVYYCGATFLRSDGHHTIWGQGT |
| | LVTVSSHHHHHH |
| AxCT mask27 EGFR EH104 | EVQLVESGGGLVQPGNSLRLSCAASGFTFSKFGMSWVRQGGG |
| L001 version 1 | GGLDGNEEPGGLEWVSSISGSGRDTLYADSVKGRFTISRDNA |
| (SEQ ID NO: 1013) | KTTLYLQMNSLRPEDTAVYYCTIGGSLSVSSQGTLVTVSSGG |
| | GGKPLGLQARVVGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L040 version 1 | GGLDGNEEPGGLEWVSSISGSGRDTLYADSVKGRFTISRDNA |
| (SEQ ID NO: 1014) | KTTLYLQMNSLRPEDTAVYYCTIGGSLSVSSQGTLVTVSSGG |
| | GGPQASTGRSGGGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L041 version 1 | GGLDGNEEPGGLEWVSSISGSGRDTLYADSVKGRFTISRDNA |
| (SEQ ID NO: 1015) | KTTLYLQMNSLRPEDTAVYYCTIGGSLSVSSQGTLVTVSSGG |
| | GGPQGSTGRAAGGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L043 version 1 | GGLDGNEEPGGLEWVSSISGSGRDTLYADSVKGRFTISRDNA |
| (SEQ ID NO: 1016) | KTTLYLQMNSLRPEDTAVYYCTIGGSLSVSSQGTLVTVSSGG |
| | GGPQGSTARSAGGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L045 version 1 | GGLDGNEEPGGLEWVSSISGSGRDTLYADSVKGRFTISRDNA |
| (SEQ ID NO: 1017) | KTTLYLQMNSLRPEDTAVYYCTIGGSLSVSSQGTLVTVSSGG |
| | GGGPIPVQGRAHGGGGTQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L054 version 1 | GGLDGNEEPGGLEWVSSISGSGRDTLYADSVKGRFTISRDNA |
| (SEQ ID NO: 1018) | KTTLYLQMNSLRPEDTAVYYCTIGGSLSVSSQGTLVTVSSGG |
| | GGVANLASTGRAGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L055 version 1 | GGLDGNEEPGGLEWVSSISGSGRDTLYADSVKGRFTISRDNA |
| (SEQ ID NO: 1019) | KTTLYLQMNSLRPEDTAVYYCTIGGSLSVSSQGTLVTVSSGG |
| | GGPLGLSTGRAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L056 version 1 | GGLDGNEEPGGLEWVSSISGSGRDTLYADSVKGRFTISRDNA |
| (SEQ ID NO: 1020) | KTTLYLQMNSLRPEDTAVYYCTIGGSLSVSSQGTLVTVSSGG |
| | GGPQASTGRAGGGTQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGGEVQLLESGGGLVQPGGSLTLSC |
| | AASGRTDSWYVMGWFRQAPGKDREFVAGVSWSYGNTYYADSV |
| | KGRFTISRDNSKNTLYLQMNSLRAEDTAVYYCAARVSREVIP |
| | TRWDLYNYWGLGTQVTVSSHHHHHH |
| AxCT mask27 EGFR EH104 | EVQLVESGGGLVQPGNSLRLSCAASGFTFSKFGMSWVRQGGG |
| L057 version 1 | GGLDGNEEPGGLEWVSSISGSGRDTLYADSVKGRFTISRDNA |
| (SEQ ID NO: 1021) | KTTLYLQMNSLRPEDTAVYYCTIGGSLSVSSQGTLVTVSSGG |
| | GGPKPQASTGRAGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L058 version 1 | GGLDGNEEPGGLEWVSSISGSGRDTLYADSVKGRFTISRDNA |
| (SEQ ID NO: 1022) | KTTLYLQMNSLRPEDTAVYYCTIGGSLSVSSQGTLVTVSSGG |
| | GGPTGLESTGRAGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L059 version 1 | GGLDGNEEPGGLEWVSSISGSGRDTLYADSVKGRFTISRDNA |
| (SEQ ID NO: 1023) | KTTLYLQMNSLRPEDTAVYYCTIGGSLSVSSQGTLVTVSSGG |
| | GGPENFFSTGRAGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L060 version 1 | GGLDGNEEPGGLEWVSSISGSGRDTLYADSVKGRFTISRDNA |
| (SEQ ID NO: 1024) | KTTLYLQMNSLRPEDTAVYYCTIGGSLSVSSQGTLVTVSSGG |
| | GGAATLKSTGRAGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L061 version 1 | GGLDGNEEPGGLEWVSSISGSGRDTLYADSVKGRFTISRDNA |
| (SEQ ID NO: 1025) | KTTLYLQMNSLRPEDTAVYYCTIGGSLSVSSQGTLVTVSSGG |
| | GGPQDLLSTGRAGGGTQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L062 version 1 | GGLDGNEEPGGLEWVSSISGSGRDTLYADSVKGRFTISRDNA |
| (SEQ ID NO: 1026) | KTTLYLQMNSLRPEDTAVYYCTIGGSLSVSSQGTLVTVSSGG |
| | GGPAGLSTGRAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L063 version 1 | GGLDGNEEPGGLEWVSSISGSGRDTLYADSVKGRFTISRDNA |
| (SEQ ID NO: 1027) | KTTLYLQMNSLRPEDTAVYYCTIGGSLSVSSQGTLVTVSSGG |
| | GGVLGLSTGRAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L064 version 1 | GGLDGNEEPGGLEWVSSISGSGRDTLYADSVKGRFTISRDNA |
| (SEQ ID NO: 1028) | KTTLYLQMNSLRPEDTAVYYCTIGGSLSVSSQGTLVTVSSGG |
| | GGVANLASTGAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L065 version 1 | GGLDGNEEPGGLEWVSSISGSGRDTLYADSVKGRFTISRDNA |
| (SEQ ID NO: 1029) | KTTLYLQMNSLRPEDTAVYYCTIGGSLSVSSQGTLVTVSSGG |
| | GGPLGLSTGAGGGTQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGGEVQLLESGGGLVQPGGSLTLSC |
| | AASGRTDSWYVMGWFRQAPGKDREFVAGVSWSYGNTYYADSV |
| | KGRFTISRDNSKNTLYLQMNSLRAEDTAVYYCAARVSREVIP |
| | TRWDLYNYWGLGTQVTVSSHHHHHH |
| AxCT mask27 EGFR EH104 | EVQLVESGGGLVQPGNSLRLSCAASGFTFSKFGMSWVRQGGG |
| L066 version 1 | GGLDGNEEPGGLEWVSSISGSGRDTLYADSVKGRFTISRDNA |
| (SEQ ID NO: 1030) | KTTLYLQMNSLRPEDTAVYYCTIGGSLSVSSQGTLVTVSSGG |
| | GGPQASTGAGGGTQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVLGGGG |
| | SGGGGSGGGGSEVQLVESGGGLVQPGGSLKLSCAASGFTFNK |
| | YAINWVRQAPGKGLEWVARIRSKYNNYATYYADQVKDRFTIS |
| | RDDSKNTAYLQMNNLKTEDTAVYYCVRHANFGNSYISYWAYW |
| | GQGTLVTVSSGGGGSGGGGEVQLLESGGGLVQPGGSLTLSCA |
| | ASGRTDSWYVMGWFRQAPGKDREFVAGVSWSYGNTYYADSVK |
| | GRFTISRDNSKNTLYLQMNSLRAEDTAVYYCAARVSREVIPT |
| | RWDLYNYWGLGTQVTVSSHHHHHH |
| AxCT mask27 EGFR EH104 | EVQLVESGGGLVQPGNSLRLSCAASGFTFSKFGMSWVRQGGG |
| L067 version 1 | GGLDGNEEPGGLEWVSSISGSGRDTLYADSVKGRFTISRDNA |
| (SEQ ID NO: 1031) | KTTLYLQMNSLRPEDTAVYYCTIGGSLSVSSQGTLVTVSSGG |
| | GGPKPQASTGAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L068 version 1 | GGLDGNEEPGGLEWVSSISGSGRDTLYADSVKGRFTISRDNA |
| (SEQ ID NO: 1032) | KTTLYLQMNSLRPEDTAVYYCTIGGSLSVSSQGTLVTVSSGG |
| | GGPTGLESTGAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L069 version 1 | GGLDGNEEPGGLEWVSSISGSGRDTLYADSVKGRFTISRDNA |
| (SEQ ID NO: 1033) | KTTLYLQMNSLRPEDTAVYYCTIGGSLSVSSQGTLVTVSSGG |
| | GGPENFFSTGAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L070 version 1 | GGLDGNEEPGGLEWVSSISGSGRDTLYADSVKGRFTISRDNA |
| (SEQ ID NO: 1034) | KTTLYLQMNSLRPEDTAVYYCTIGGSLSVSSQGTLVTVSSGG |
| | GGAATLKSTGAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L071 version 1 | GGLDGNEEPGGLEWVSSISGSGRDTLYADSVKGRFTISRDNA |
| (SEQ ID NO: 1035) | KTTLYLQMNSLRPEDTAVYYCTIGGSLSVSSQGTLVTVSSGG |
| | GGPQDLLSTGAGGGTQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L072 version 1 | GGLDGNEEPGGLEWVSSISGSGRDTLYADSVKGRFTISRDNA |
| (SEQ ID NO: 1036) | KTTLYLQMNSLRPEDTAVYYCTIGGSLSVSSQGTLVTVSSGG |
| | GGPAGLSTGAGGGTQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGGEVQLLESGGGLVQPGGSLTLSC |
| | AASGRTDSWYVMGWFRQAPGKDREFVAGVSWSYGNTYYADSV |
| | KGRFTISRDNSKNTLYLQMNSLRAEDTAVYYCAARVSREVIP |
| | TRWDLYNYWGLGTQVTVSSHHHHHH |
| AxCT mask27 EGFR EH104 | EVQLVESGGGLVQPGNSLRLSCAASGFTFSKFGMSWVRQGGG |
| L073 version 1 | GGLDGNEEPGGLEWVSSISGSGRDTLYADSVKGRFTISRDNA |
| (SEQ ID NO: 1037) | KTTLYLQMNSLRPEDTAVYYCTIGGSLSVSSQGTLVTVSSGG |
| | GGVLGLSTGAGGGTQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGGEVQLLESGGGLVQPGGSLTLSC |
| | AASGRTDSWYVMGWFRQAPGKDREFVAGVSWSYGNTYYADSV |
| | KGRFTISRDNSKNTLYLQMNSLRAEDTAVYYCAARVSREVIP |
| | TRWDLYNYWGLGTQVTVSSHHHHHH |
| AxCT mask27 EpCAM H90.2 | EVQLVESGGGLVQPGNSLRLSCAASGFTFSKFGMSWVRQGGG |
| L040 version 2 | GGLDGNEEPGGLEWVSSISGSGRDTLYADSVKGRFTISRDNA |
| (SEQ ID NO: 1038) | KTTLYLQMNSLRPEDTAVYYCTIGGSLSVSSQGTLVTVSSGG |
| | GGPQASTGRSGGGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTIWGQGTLVTVSSHHHHHH |
| AxCT mask27 EpCAM H90.2 | EVQLVESGGGLVQPGNSLRLSCAASGFTFSKFGMSWVRQGGG |
| L041 version 2 | GGLDGNEEPGGLEWVSSISGSGRDTLYADSVKGRFTISRDNA |
| (SEQ ID NO: 1039) | KTTLYLQMNSLRPEDTAVYYCTIGGSLSVSSQGTLVTVSSGG |
| | GGPQGSTGRAAGGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTIWGQGTLVTVSSHHHHHH |
| AxCT mask27 EpCAM H90.2 | EVQLVESGGGLVQPGNSLRLSCAASGFTFSKFGMSWVRQGGG |
| L043 version 2 | GGLDGNEEPGGLEWVSSISGSGRDTLYADSVKGRFTISRDNA |
| (SEQ ID NO: 1040) | KTTLYLQMNSLRPEDTAVYYCTIGGSLSVSSQGTLVTVSSGG |
| | GGPQGSTARSAGGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVEGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTIWGQGTLVTVSSHHHHHH |
| AxCT mask27 EpCAM H90.2 | EVQLVESGGGLVQPGNSLRLSCAASGFTFSKFGMSWVRQGGG |
| L076 version 2 | GGLDGNEEPGGLEWVSSISGSGRDTLYADSVKGRFTISRDNA |
| (SEQ ID NO: 1041) | KTTLYLQMNSLRPEDTAVYYCTIGGSLSVSSQGTLVTVSSGG |
| | GGPQASTGGSGGGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTIWGQGTLVTVSSHHHHHH |
| AxCT mask27 EpCAM H90.2 | EVQLVESGGGLVQPGNSLRLSCAASGFTFSKFGMSWVRQGGG |
| L059 version 2 | GGLDGNEEPGGLEWVSSISGSGRDTLYADSVKGRFTISRDNA |
| (SEQ ID NO: 1042) | KTTLYLQMNSLRPEDTAVYYCTIGGSLSVSSQGTLVTVSSGG |
| | GGPENFFSTGRAGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGSEVQLLESGGGLVQPGGSLTL |
| | SCAASGFIFRAASMAWYRQSPGNERELVASISSGAFTNYADS |
| | VKARFTISRDNSKNTLYLQMNSLRAEDTAVYYCGATFLRSDG |
| | HHTIWGQGTLVTVSSHHHHHH |
| AxCT mask27 EpCAM H90.2 | EVQLVESGGGLVQPGNSLRLSCAASGFTFSKFGMSWVRQGGG |
| L060 version 2 | GGLDGNEEPGGLEWVSSISGSGRDTLYADSVKGRFTISRDNA |
| (SEQ ID NO: 1043) | KTTLYLQMNSLRPEDTAVYYCTIGGSLSVSSQGTLVTVSSGG |
| | GGAATLKSTGRAGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGSEVQLLESGGGLVQPGGSLTL |
| | SCAASGFIFRAASMAWYRQSPGNERELVASISSGAFTNYADS |
| | VKARFTISRDNSKNTLYLQMNSLRAEDTAVYYCGATFLRSDG |
| | HHTIWGQGTLVTVSSHHHHHH |
| AxCT mask27 EpCAM H90.2 | EVQLVESGGGLVQPGNSLRLSCAASGFTFSKFGMSWVRQGGG |
| L061 version 2 | GGLDGNEEPGGLEWVSSISGSGRDTLYADSVKGRFTISRDNA |
| (SEQ ID NO: 1044) | KTTLYLQMNSLRPEDTAVYYCTIGGSLSVSSQGTLVTVSSGG |
| | GGPQDLLSTGRAGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGSEVQLLESGGGLVQPGGSLTL |
| | SCAASGFIFRAASMAWYRQSPGNERELVASISSGAFTNYADS |
| | VKARFTISRDNSKNTLYLQMNSLRAEDTAVYYCGATFLRSDG |
| | HHTIWGQGTLVTVSSHHHHHH |
| AxCT mask27 EpCAM H90.2 | EVQLVESGGGLVQPGNSLRLSCAASGFTFSKFGMSWVRQGGG |
| L063 version 2 | GGLDGNEEPGGLEWVSSISGSGRDTLYADSVKGRFTISRDNA |
| (SEQ ID NO: 1045) | KTTLYLQMNSLRPEDTAVYYCTIGGSLSVSSQGTLVTVSSGG |
| | GGVLGLSTGRAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGSEVQLLESGGGLVQPGGSLTLS |
| | CAASGFIFRAASMAWYRQSPGNERELVASISSGAFTNYADSV |
| | KARFTISRDNSKNTLYLQMNSLRAEDTAVYYCGATFLRSDGH |
| | HTIWGQGTLVTVSSHHHHHH |
| AxCT mask27 EpCAM H90. 2 | EVQLVESGGGLVQPGNSLRLSCAASGFTFSKFGMSWVRQGGG |
| L064 version 2 | GGLDGNEEPGGLEWVSSISGSGRDTLYADSVKGRFTISRDNA |
| (SEQ ID NO: 1046) | KTTLYLQMNSLRPEDTAVYYCTIGGSLSVSSQGTLVTVSSGG |
| | GGVANLASTGAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGSEVQLLESGGGLVQPGGSLTLS |
| | CAASGFIFRAASMAWYRQSPGNERELVASISSGAFTNYADSV |
| | KARFTISRDNSKNTLYLQMNSLRAEDTAVYYCGATFLRSDGH |
| | HTIWGQGTLVTVSSHHHHHH |
| AxCT mask27 EpCAM H90.2 | EVQLVESGGGLVQPGNSLRLSCAASGFTFSKFGMSWVRQGGG |
| L073 version 2 | GGLDGNEEPGGLEWVSSISGSGRDTLYADSVKGRFTISRDNA |
| (SEQ ID NO: 1047) | KTTLYLQMNSLRPEDTAVYYCTIGGSLSVSSQGTLVTVSSGG |
| | GGVLGLSTGAGGGSQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGSEVQLLESGGGLVQPGGSLTLSC |
| | AASGFIFRAASMAWYRQSPGNERELVASISSGAFTNYADSVK |
| | ARFTISRDNSKNTLYLQMNSLRAEDTAVYYCGATFLRSDGHH |
| | TIWGQGTLVTVSSHHHHHH |
| AxCT mask27 EpCAM H90.2 | EVQLVESGGGLVQPGNSLRLSCAASGFTFSKFGMSWVRQGGG |
| L075 version 2 | GGLDGNEEPGGLEWVSSISGSGRDTLYADSVKGRFTISRDNA |
| (SEQ ID NO: 1048) | KTTLYLQMNSLRPEDTAVYYCTIGGSLSVSSQGTLVTVSSGG |
| | GGRTRYEDGGSGGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGSEVQLLESGGGLVQPGGSLTL |
| | SCAASGFIFRAASMAWYRQSPGNERELVASISSGAFTNYADS |
| | VKARFTISRDNSKNTLYLQMNSLRAEDTAVYYCGATFLRSDG |
| | HHTIWGQGTLVTVSSHHHHHH |
| AxCT mask27 EpCAM H90 | EVQLVESGGGLVQPGNSLRLSCAASGFTFSKFGMSWVRQGGG |
| L001 version 2 | GGLDGNEEPGGLEWVSSISGSGRDTLYADSVKGRFTISRDNA |
| (SEQ ID NO: 1049) | KTTLYLQMNSLRPEDTAVYYCTIGGSLSVSSQGTLVTVSSGG |
| | GGKPLGLQARVVGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGSEVQLLESGGGLVQPGGSLT |
| | LSCAASGFIFRAASMAWYRQSPGNERELVASISSGAFTNYAD |
| | SVKARFTISRDNSKNTLYLQMNSLRAEDTAVYYCGATFLRSD |
| | GHHTINGQGTLVTVSSHHHHHH |
| AxCT mask27 EGFR EH104 | EVQLVESGGGLVQPGNSLRLSCAASGFTFSKFGMSWVRQGGG |
| L001 version 2 | GGLDGNEEPGGLEWVSSISGSGRDTLYADSVKGRFTISRDNA |
| (SEQ ID NO: 1050) | KTTLYLQMNSLRPEDTAVYYCTIGGSLSVSSQGTLVTVSSGG |
| | GGKPLGLQARVVGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L040 version 2 | GGLDGNEEPGGLEWVSSISGSGRDTLYADSVKGRFTISRDNA |
| (SEQ ID NO: 1051) | KTTLYLQMNSLRPEDTAVYYCTIGGSLSVSSQGTLVTVSSGG |
| | GGPQASTGRSGGGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L041 version 2 | GGLDGNEEPGGLEWVSSISGSGRDTLYADSVKGRFTISRDNA |
| (SEQ ID NO: 1052) | KTTLYLQMNSLRPEDTAVYYCTIGGSLSVSSQGTLVTVSSGG |
| | GGPQGSTGRAAGGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L043 version 2 | GGLDGNEEPGGLEWVSSISGSGRDTLYADSVKGRFTISRDNA |
| (SEQ ID NO: 1053) | KTTLYLQMNSLRPEDTAVYYCTIGGSLSVSSQGTLVTVSSGG |
| | GGPQGSTARSAGGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L045 version 2 | GGLDGNEEPGGLEWVSSISGSGRDTLYADSVKGRFTISRDNA |
| (SEQ ID NO: 1054) | KTTLYLQMNSLRPEDTAVYYCTIGGSLSVSSQGTLVTVSSGG |
| | GGGPIPVQGRAHGGGGSQTVVTQEPSLTVSPGGTVTLTCASS |
| | TGAVTSGNYPNWVQQKPGQAPRGLIGGTKFLVPGTPARFSGS |
| | LLGGKAALTLSGVQPEDEAEYYCTLWYSNRWVFGGGTKLTVL |
| | GGGGSGGGGSGGGGSEVQLVESGGGLVQPGGSLKLSCAASGF |
| | TFNKYAINWVRQAPGKGLEWVARIRSKYNNYATYYADQVKDR |
| | FTISRDDSKNTAYLQMNNLKTEDTAVYYCVRHANFGNSYISY |
| | WAYWGQGTLVTVSSGGGGSGGGGEVQLLESGGGLVQPGGSLT |
| | LSCAASGRTDSWYVMGWFRQAPGKDREFVAGVSWSYGNTYYA |
| | DSVKGRFTISRDNSKNTLYLQMNSLRAEDTAVYYCAARVSRE |
| | VIPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L054 version 2 | GGLDGNEEPGGLEWVSSISGSGRDTLYADSVKGRFTISRDNA |
| (SEQ ID NO: 1055) | KTTLYLQMNSLRPEDTAVYYCTIGGSLSVSSQGTLVTVSSGG |
| | GGVANLASTGRAGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L055 version 2 | GGLDGNEEPGGLEWVSSISGSGRDTLYADSVKGRFTISRDNA |
| (SEQ ID NO: 1056) | KTTLYLQMNSLRPEDTAVYYCTIGGSLSVSSQGTLVTVSSGG |
| | GGPLGLSTGRAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L056 version 2 | GGLDGNEEPGGLEWVSSISGSGRDTLYADSVKGRFTISRDNA |
| (SEQ ID NO: 1057) | KTTLYLQMNSLRPEDTAVYYCTIGGSLSVSSQGTLVTVSSGG |
| | GGPQASTGRAGGGSQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGGEVQLLESGGGLVQPGGSLTLSC |
| | AASGRTDSWYVMGWFRQAPGKDREFVAGVSWSYGNTYYADSV |
| | KGRFTISRDNSKNTLYLQMNSLRAEDTAVYYCAARVSREVIP |
| | TRWDLYNYWGLGTQVTVSSHHHHHH |
| AxCT mask27 EGFR EH104 | EVQLVESGGGLVQPGNSLRLSCAASGFTFSKFGMSWVRQGGG |
| L057 version 2 | GGLDGNEEPGGLEWVSSISGSGRDTLYADSVKGRFTISRDNA |
| (SEQ ID NO: 1058) | KTTLYLQMNSLRPEDTAVYYCTIGGSLSVSSQGTLVTVSSGG |
| | GGPKPQASTGRAGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L058 version 2 | GGLDGNEEPGGLEWVSSISGSGRDTLYADSVKGRFTISRDNA |
| (SEQ ID NO: 1059) | KTTLYLQMNSLRPEDTAVYYCTIGGSLSVSSQGTLVTVSSGG |
| | GGPTGLESTGRAGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L059 version 2 | GGLDGNEEPGGLEWVSSISGSGRDTLYADSVKGRFTISRDNA |
| (SEQ ID NO: 1060) | KTTLYLQMNSLRPEDTAVYYCTIGGSLSVSSQGTLVTVSSGG |
| | GGPENFFSTGRAGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L060 version 2 | GGLDGNEEPGGLEWVSSISGSGRDTLYADSVKGRFTISRDNA |
| (SEQ ID NO: 1061) | KTTLYLQMNSLRPEDTAVYYCTIGGSLSVSSQGTLVTVSSGG |
| | GGAATLKSTGRAGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L061 version 2 | GGLDGNEEPGGLEWVSSISGSGRDTLYADSVKGRFTISRDNA |
| (SEQ ID NO: 1062) | KTTLYLQMNSLRPEDTAVYYCTIGGSLSVSSQGTLVTVSSGG |
| | GGPQDLLSTGRAGGGSQTVVTQEPSLTVSPGGTVTLTCASST |
| | GAVTSGNYPNWVQQKPGQAPRGLIGGTKFLVPGTPARFSGSL |
| | LGGKAALTLSGVQPEDEAEYYCTLWYSNRWVFGGGTKLTVLG |
| | GGGSGGGGSGGGGSEVQLVESGGGLVQPGGSLKLSCAASGFT |
| | FNKYAINWVRQAPGKGLEWVARIRSKYNNYATYYADQVKDRF |
| | TISRDDSKNTAYLQMNNLKTEDTAVYYCVRHANFGNSYISYW |
| | AYWGQGTLVTVSSGGGGSGGGGEVQLLESGGGLVQPGGSLTL |
| | SCAASGRTDSWYVMGWFRQAPGKDREFVAGVSWSYGNTYYAD |
| | SVKGRFTISRDNSKNTLYLQMNSLRAEDTAVYYCAARVSREV |
| | IPTRWDLYNYWGLGTQVTVSSHHHHHH |
| AxCT mask27 EGFR EH104 | EVQLVESGGGLVQPGNSLRLSCAASGFTFSKFGMSWVRQGGG |
| L062 version 2 | GGLDGNEEPGGLEWVSSISGSGRDTLYADSVKGRFTISRDNA |
| (SEQ ID NO: 1063) | KTTLYLQMNSLRPEDTAVYYCTIGGSLSVSSQGTLVTVSSGG |
| | GGPAGLSTGRAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L063 version 2 | GGLDGNEEPGGLEWVSSISGSGRDTLYADSVKGRFTISRDNA |
| (SEQ ID NO: 1064) | KTTLYLQMNSLRPEDTAVYYCTIGGSLSVSSQGTLVTVSSGG |
| | GGVLGLSTGRAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L064 version 2 | GGLDGNEEPGGLEWVSSISGSGRDTLYADSVKGRFTISRDNA |
| (SEQ ID NO: 1065) | KTTLYLQMNSLRPEDTAVYYCTIGGSLSVSSQGTLVTVSSGG |
| | GGVANLASTGAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L065 version 2 | GGLDGNEEPGGLEWVSSISGSGRDTLYADSVKGRFTISRDNA |
| (SEQ ID NO: 1066) | KTTLYLQMNSLRPEDTAVYYCTIGGSLSVSSQGTLVTVSSGG |
| | GGPLGLSTGAGGGSQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGGEVQLLESGGGLVQPGGSLTLSC |
| | AASGRTDSWYVMGWFRQAPGKDREFVAGVSWSYGNTYYADSV |
| | KGRFTISRDNSKNTLYLQMNSLRAEDTAVYYCAARVSREVIP |
| | TRWDLYNYWGLGTQVTVSSHHHHHH |
| AxCT mask27 EGFR EH104 | EVQLVESGGGLVQPGNSLRLSCAASGFTFSKFGMSWVRQGGG |
| L066 version 2 | GGLDGNEEPGGLEWVSSISGSGRDTLYADSVKGRFTISRDNA |
| (SEQ ID NO: 1067) | KTTLYLQMNSLRPEDTAVYYCTIGGSLSVSSQGTLVTVSSGG |
| | GGPQASTGAGGGSQTVVTQEPSLTVSPGGTVTLTCASSTGAV |
| | TSGNYPNWVQQKPGQAPRGLIGGTKFLVPGTPARFSGSLLGG |
| | KAALTLSGVQPEDEAEYYCTLWYSNRWVFGGGTKLTVLGGGG |
| | SGGGGSGGGGSEVQLVESGGGLVQPGGSLKLSCAASGFTFNK |
| | YAINWVRQAPGKGLEWVARIRSKYNNYATYYADQVKDRETIS |
| | RDDSKNTAYLQMNNLKTEDTAVYYCVRHANFGNSYISYWAYW |
| | GQGTLVTVSSGGGGSGGGGEVQLLESGGGLVQPGGSLTLSCA |
| | ASGRTDSWYVMGWFRQAPGKDREFVAGVSWSYGNTYYADSVK |
| | GRFTISRDNSKNTLYLQMNSLRAEDTAVYYCAARVSREVIPT |
| | RWDLYNYWGLGTQVTVSSHHHHHH |
| AxCT mask27 EGFR EH104 | EVQLVESGGGLVQPGNSLRLSCAASGFTFSKFGMSWVRQGGG |
| L067 version 2 | GGLDGNEEPGGLEWVSSISGSGRDTLYADSVKGRFTISRDNA |
| (SEQ ID NO: 1068) | KTTLYLQMNSLRPEDTAVYYCTIGGSLSVSSQGTLVTVSSGG |
| | GGPKPQASTGAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L068 version 2 | GGLDGNEEPGGLEWVSSISGSGRDTLYADSVKGRFTISRDNA |
| (SEQ ID NO: 1069) | KTTLYLQMNSLRPEDTAVYYCTIGGSLSVSSQGTLVTVSSGG |
| | GGPTGLESTGAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L069 version 2 | GGLDGNEEPGGLEWVSSISGSGRDTLYADSVKGRFTISRDNA |
| (SEQ ID NO: 1070) | KTTLYLQMNSLRPEDTAVYYCTIGGSLSVSSQGTLVTVSSGG |
| | GGPENFFSTGAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L070 version 2 | GGLDGNEEPGGLEWVSSISGSGRDTLYADSVKGRFTISRDNA |
| (SEQ ID NO: 1071) | KTTLYLQMNSLRPEDTAVYYCTIGGSLSVSSQGTLVTVSSGG |
| | GGAATLKSTGAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L071 version 2 | GGLDGNEEPGGLEWVSSISGSGRDTLYADSVKGRFTISRDNA |
| (SEQ ID NO: 1072) | KTTLYLQMNSLRPEDTAVYYCTIGGSLSVSSQGTLVTVSSGG |
| | GGPQDLLSTGAGGGSQTVVTQEPSLTVSPGGTVTLTCASSTG |
| | AVTSGNYPNWVQQKPGQAPRGLIGGTKFLVPGTPARFSGSLL |
| | GGKAALTLSGVQPEDEAEYYCTLWYSNRWVFGGGTKLTVLGG |
| | GGSGGGGSGGGGSEVQLVESGGGLVQPGGSLKLSCAASGFTF |
| | NKYAINWVRQAPGKGLEWVARIRSKYNNYATYYADQVKDRFT |
| | ISRDDSKNTAYLQMNNLKTEDTAVYYCVRHANFGNSYISYWA |
| | YWGQGTLVTVSSGGGGSGGGGEVQLLESGGGLVQPGGSLTLS |
| | CAASGRTDSWYVMGWFRQAPGKDREFVAGVSWSYGNTYYADS |
| | VKGRFTISRDNSKNTLYLQMNSLRAEDTAVYYCAARVSREVI |
| | PTRWDLYNYWGLGTQVTVSSHHHHHH |
| AxCT mask27 EGFR EH104 | EVQLVESGGGLVQPGNSLRLSCAASGFTFSKFGMSWVRQGGG |
| L072 version 2 | GGLDGNEEPGGLEWVSSISGSGRDTLYADSVKGRFTISRDNA |
| (SEQ ID NO: 1073) | KTTLYLQMNSLRPEDTAVYYCTIGGSLSVSSQGTLVTVSSGG |
| | GGPAGLSTGAGGGSQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGGEVQLLESGGGLVQPGGSLTLSC |
| | AASGRTDSWYVMGWFRQAPGKDREFVAGVSWSYGNTYYADSV |
| | KGRFTISRDNSKNTLYLQMNSLRAEDTAVYYCAARVSREVIP |
| | TRWDLYNYWGLGTQVTVSSHHHHHH |
| AxCT mask27 EGFR EH104 | EVQLVESGGGLVQPGNSLRLSCAASGFTFSKFGMSWVRQGGG |
| L073 version 2 | GGLDGNEEPGGLEWVSSISGSGRDTLYADSVKGRFTISRDNA |
| (SEQ ID NO: 1074) | KTTLYLQMNSLRPEDTAVYYCTIGGSLSVSSQGTLVTVSSGG |
| | GGVLGLSTGAGGGSQTVVTQEPSLTVSPGGTVTLTCASSTGA |
| | VTSGNYPNWVQQKPGQAPRGLIGGTKFLVPGTPARFSGSLLG |
| | GKAALTLSGVQPEDEAEYYCTLWYSNRWVFGGGTKLTVLGGG |
| | GSGGGGSGGGGSEVQLVESGGGLVQPGGSLKLSCAASGFTFN |
| | KYAINWVRQAPGKGLEWVARIRSKYNNYATYYADQVKDRFTI |
| | SRDDSKNTAYLQMNNLKTEDTAVYYCVRHANFGNSYISYWAY |
| | WGQGTLVTVSSGGGGSGGGGEVQLLESGGGLVQPGGSLTLSC |
| | AASGRTDSWYVMGWFRQAPGKDREFVAGVSWSYGNTYYADSV |
| | KGRFTISRDNSKNTLYLQMNSLRAEDTAVYYCAARVSREVIP |
| | TRWDLYNYWGLGTQVTVSSHHHHHH |
| Construct C2483 (SEQ ID | MALPVTALLLPLALLLHAARPEVQLLESGGGLVQPGGSLTLS |
| NO: 1075) | CAASGFIFRAASMAWYRQSPGNERELVASISSGAFTNYADSV |
| | KARFTISRDNSKNTLYLQMNSLRAEDTAVYYCGATFLRSDGH |
| | HTINGQGTLVTVSSTSDYKDDDDKTTTPAPRPPTPAPTIASQ |
| | PLSLRPEACRPAAGGAVHTRGLDFACDIYIWAPLAGTCGVLL |
| | LSLVITLYKRGRKKLLYIFKQPFMRPVQTTQEEDGCSCRFPE |
| | EEEGGCELRVKFSRSADAPAYQQGQNQLYNELNLGRREEYDV |
| | LDKRRGRDPEMGGKPRRKNPQEGLYNELQKDKMAEAYSEIGM |
| | KGERRRGKGHDGLYQGLSTATKDTYDALHMQALPPR |
| Construct C3544 (SEQ ID | MALPVTALLLPLALLLHAARPEQKLISEEDLEVQLVESGGGL |
| NO: 1076) | VQPGNSLRLSCAASGFTFSKFGMSWVRQRQSTFMAMDMPILE |
| | WVSSISGSGRDTLYADSVKGRFTISRDNAKTTLYLQMNSLRP |
| | EDTAVYYCTIGGSLSVSSQGTLVTVSSGGGGSGGGQAGVVGG |
| | GGTEVQLLESGGGLVQPGGSLTLSCAASGFIFRAASMAWYRQ |
| | SPGNERELVASISSGAFTNYADSVKARFTISRDNSKNTLYLQ |
| | MNSLRAEDTAVYYCGATFLRSDGHHTINGQGTLVTVSSTSDY |
| | KDDDDKTTTPAPRPPTPAPTIASQPLSLRPEACRPAAGGAVH |
| | TRGLDFACDIYIWAPLAGTCGVLLLSLVITLYKRGRKKLLYI |
| | FKQPFMRPVQTTQEEDGCSCRFPEEEEGGCELRVKFSRSADA |
| | PAYQQGQNQLYNELNLGRREEYDVLDKRRGRDPEMGGKPRRK |
| | NPQEGLYNELQKDKMAEAYSEIGMKGERRRGKGHDGLYQGLS |
| | TATKDTYDALHMQALPPR |
| protease cleavable | GGGGPLGLQAGVVGGGGT |
| linker 4 (SEQ ID NO: | |
| 1077) | |
| Construct C3546 (SEQ ID | MALPVTALLLPLALLLHAARPEQKLISEEDLEVQLVESGGGL |
| NO: 1078) | VQPGNSLRLSCAASGFTFSKFGMSWVRQRQSTFMAMDMPILE |
| | WVSSISGSGRDTLYADSVKGRFTISRDNAKTTLYLQMNSLRP |
| | EDTAVYYCTIGGSLSVSSQGTLVTVSSGGGGPLGLQAGVVGG |
| | GGTEVQLLESGGGLVQPGGSLTLSCAASGFIFRAASMAWYRQ |
| | SPGNERELVASISSGAFTNYADSVKARFTISRDNSKNTLYLQ |
| | MNSLRAEDTAVYYCGATFLRSDGHHTINGQGTLVTVSSTSDY |
| | KDDDDKTTTPAPRPPTPAPTIASQPLSLRPEACRPAAGGAVH |
| | TRGLDFACDIYIWAPLAGTCGVLLLSLVITLYKRGRKKLLYI |
| | FKQPFMRPVQTTQEEDGCSCRFPEEEEGGCELRVKFSRSADA |
| | PAYQQGQNQLYNELNLGRREEYDVLDKRRGRDPEMGGKPRRK |
| | NPQEGLYNELQKDKMAEAYSEIGMKGERRRGKGHDGLYQGLS |
| | TATKDTYDALHMQALPPR |
| protease cleavable | GGGGPLNLQAGVVGGGGT |
| linker 5 (SEQ ID NO: | |
| 1079) | |
| Construct C3548 (SEQ ID | MALPVTALLLPLALLLHAARPEQKLISEEDLEVQLVESGGGL |
| NO: 1080) | VQPGNSLRLSCAASGFTFSKFGMSWVRQRQSTFMAMDMPILE |
| | WVSSISGSGRDTLYADSVKGRFTISRDNAKTTLYLQMNSLRP |
| | EDTAVYYCTIGGSLSVSSQGTLVTVSSGGGGPLNLQAGVVGG |
| | GGTEVQLLESGGGLVQPGGSLTLSCAASGFIFRAASMAWYRQ |
| | SPGNERELVASISSGAFTNYADSVKARFTISRDNSKNTLYLQ |
| | MNSLRAEDTAVYYCGATFLRSDGHHTINGQGTLVTVSSTSDY |
| | KDDDDKTTTPAPRPPTPAPTIASQPLSLRPEACRPAAGGAVH |
| | TRGLDFACDIYIWAPLAGTCGVLLLSLVITLYKRGRKKLLYI |
| | FKQPFMRPVQTTQEEDGCSCRFPEEEEGGCELRVKFSRSADA |
| | PAYQQGQNQLYNELNLGRREEYDVLDKRRGRDPEMGGKPRRK |
| | NPQEGLYNELQKDKMAEAYSEIGMKGERRRGKGHDGLYQGLS |
| | TATKDTYDALHMQALPPR |
| protease cleavable | GGGGPAGLQAGVVGGGGT |
| linker 6 (SEQ ID NO: | |
| 1081) | |
| Construct C3549 (SEQ ID | MALPVTALLLPLALLLHAARPEQKLISEEDLEVQLVESGGGL |
| NO: 1082) | VQPGNSLRLSCAASGFTFSKFGMSWVRQRQSTFMAMDMPILE |
| | WVSSISGSGRDTLYADSVKGRFTISRDNAKTTLYLQMNSLRP |
| | EDTAVYYCTIGGSLSVSSQGTLVTVSSGGGGPAGLQAGVVGG |
| | GGTEVQLLESGGGLVQPGGSLTLSCAASGFIFRAASMAWYRQ |
| | SPGNERELVASISSGAFTNYADSVKARFTISRDNSKNTLYLQ |
| | MNSLRAEDTAVYYCGATFLRSDGHHTINGQGTLVTVSSTSDY |
| | KDDDDKTTTPAPRPPTPAPTIASQPLSLRPEACRPAAGGAVH |
| | TRGLDFACDIYIWAPLAGTCGVLLLSLVITLYKRGRKKLLYI |
| | FKQPFMRPVQTTQEEDGCSCRFPEEEEGGCELRVKFSRSADA |
| | PAYQQGQNQLYNELNLGRREEYDVLDKRRGRDPEMGGKPRRK |
| | NPQEGLYNELQKDKMAEAYSEIGMKGERRRGKGHDGLYQGLS |
| | TATKDTYDALHMQALPPR |
| protease cleavable | GGGGPANLQAGVVGGGGT |
| linker 7 (SEQ ID NO: | |
| 1083) | |
| Construct C3550 (SEQ ID | MALPVTALLLPLALLLHAARPEQKLISEEDLEVQLVESGGGL |
| NO: 1084) | VQPGNSLRLSCAASGFTFSKFGMSWVRQRQSTFMAMDMPILE |
| | WVSSISGSGRDTLYADSVKGRFTISRDNAKTTLYLQMNSLRP |
| | EDTAVYYCTIGGSLSVSSQGTLVTVSSGGGGPANLQAGVVGG |
| | GGTEVQLLESGGGLVQPGGSLTLSCAASGFIFRAASMAWYRQ |
| | SPGNERELVASISSGAFTNYADSVKARFTISRDNSKNTLYLQ |
| | MNSLRAEDTAVYYCGATFLRSDGHHTINGQGTLVTVSSTSDY |
| | KDDDDKTTTPAPRPPTPAPTIASQPLSLRPEACRPAAGGAVH |
| | TRGLDFACDIYIWAPLAGTCGVLLLSLVITLYKRGRKKLLYI |
| | FKQPFMRPVQTTQEEDGCSCRFPEEEEGGCELRVKFSRSADA |
| | PAYQQGQNQLYNELNLGRREEYDVLDKRRGRDPEMGGKPRRK |
| | NPQEGLYNELQKDKMAEAYSEIGMKGERRRGKGHDGLYQGLS |
| | TATKDTYDALHMQALPPR |
| Construct C3269 (SEQ ID | MALPVTALLLPLALLLHAARPEVQLVESGGGLVQPGNSLRLS |
| NO: 1085) | CAASGFTFSKFGMSWVRQRQSTFMAMDMPILEWVSSISGSGR |
| | DTLYADSVKGRFTISRDNAKTTLYLQMNSLRPEDTAVYYCTI |
| | GGSLSVSSQGTLVTVSSGSGGSGGGGSGGGGGSGEVQLLESG |
| | GGLVQPGGSLTLSCAASGFIFRAASMAWYRQSPGNERELVAS |
| | ISSGAFTNYADSVKARFTISRDNSKNTLYLQMNSLRAEDTAV |
| | YYCGATFLRSDGHHTINGQGTLVTVSSTSDYKDDDDKTTTPA |
| | PRPPTPAPTIASQPLSLRPEACRPAAGGAVHTRGLDFACDIY |
| | IWAPLAGTCGVLLLSLVITLYKRGRKKLLYIFKQPFMRPVQT |
| | TQEEDGCSCRFPEEEEGGCELRVKFSRSADAPAYQQGQNQLY |
| | NELNLGRREEYDVLDKRRGRDPEMGGKPRRKNPQEGLYNELQ |
| | KDKMAEAYSEIGMKGERRRGKGHDGLYQGLSTATKDTYDALH |
| | MQALPPR |
| protease cleavable | GGGGEYSGLSDGGGGGT |
| linker 8 (SEQ ID NO: | |
| 1086) | |
| Construct C3440 (SEQ ID | MALPVTALLLPLALLLHAARPEVQLVESGGGLVQPGNSLRLS |
| NO: 1087) | CAASGFTFSKFGMSWVRQRQSTFMAMDMPILEWVSSISGSGR |
| | DTLYADSVKGRFTISRDNAKTTLYLQMNSLRPEDTAVYYCTI |
| | GGSLSVSSQGTLVTVSSGGGGEYSGLSDGGGGGTEVQLLESG |
| | GGLVQPGGSLTLSCAASGFIFRAASMAWYRQSPGNERELVAS |
| | ISSGAFTNYADSVKARFTISRDNSKNTLYLQMNSLRAEDTAV |
| | YYCGATFLRSDGHHTINGQGTLVTVSSTSDYKDDDDKTTTPA |
| | PRPPTPAPTIASQPLSLRPEACRPAAGGAVHTRGLDFACDIY |
| | IWAPLAGTCGVLLLSLVITLYKRGRKKLLYIFKQPFMRPVQT |
| | TQEEDGCSCRFPEEEEGGCELRVKFSRSADAPAYQQGQNQLY |
| | NELNLGRREEYDVLDKRRGRDPEMGGKPRRKNPQEGLYNELQ |
| | KDKMAEAYSEIGMKGERRRGKGHDGLYQGLSTATKDTYDALH |
| | MQALPPR |
| protease cleavable | GGGGKPAEFTVDGGGGT |
| linker 9 (SEQ ID NO: | |
| 1088) | |
| protease cleavable | GGGGAISELFAKGGGGT |
| linker 10 (SEQ ID NO: | |
| 1089) | |
| Construct C3543 (SEQ ID | MALPVTALLLPLALLLHAARPEQKLISEEDLEVQLVESGGGL |
| NO: 1090) | VQPGNSLRLSCAASGFTFSKFGMSWVRQRQSTFMAMDMPILE |
| | WVSSISGSGRDTLYADSVKGRFTISRDNAKTTLYLQMNSLRP |
| | EDTAVYYCTIGGSLSVSSQGTLVTVSSGGGGSGGGGSGGGGG |
| | GGTEVQLLESGGGLVQPGGSLTLSCAASGFIFRAASMAWYRQ |
| | SPGNERELVASISSGAFTNYADSVKARFTISRDNSKNTLYLQ |
| | MNSLRAEDTAVYYCGATFLRSDGHHTINGQGTLVTVSSTSDY |
| | KDDDDKTTTPAPRPPTPAPTIASQPLSLRPEACRPAAGGAVH |
| | TRGLDFACDIYIWAPLAGTCGVLLLSLVITLYKRGRKKLLYI |
| | FKQPFMRPVQTTQEEDGCSCRFPEEEEGGCELRVKFSRSADA |
| | PAYQQGQNQLYNELNLGRREEYDVLDKRRGRDPEMGGKPRRK |
| | NPQEGLYNELQKDKMAEAYSEIGMKGERRRGKGHDGLYQGLS |
| | TATKDTYDALHMQALPPR |
| protease cleavable | GGGGGDFLAEGGGGT |
| linker 11 (SEQ ID NO: | |
| 1091) | |
| Construct C3558 (SEQ ID | MALPVTALLLPLALLLHAARPEQKLISEEDLEVQLVESGGGL |
| NO: 1092) | VQPGNSLRLSCAASGFTFSKFGMSWVRQRQSTFMAMDMPILE |
| | WVSSISGSGRDTLYADSVKGRFTISRDNAKTTLYLQMNSLRP |
| | EDTAVYYCTIGGSLSVSSQGTLVTVSSGGGGGDFLAEGGGGT |
| | EVQLLESGGGLVQPGGSLTLSCAASGFIFRAASMAWYRQSPG |
| | NERELVASISSGAFTNYADSVKARFTISRDNSKNTLYLQMNS |
| | LRAEDTAVYYCGATFLRSDGHHTINGQGTLVTVSSTSDYKDD |
| | DDKTTTPAPRPPTPAPTIASQPLSLRPEACRPAAGGAVHTRG |
| | LDFACDIYIWAPLAGTCGVLLLSLVITLYKRGRKKLLYIFKQ |
| | PFMRPVQTTQEEDGCSCRFPEEEEGGCELRVKFSRSADAPAY |
| | QQGQNQLYNELNLGRREEYDVLDKRRGRDPEMGGKPRRKNPQ |
| | EGLYNELQKDKMAEAYSEIGMKGERRRGKGHDGLYQGLSTAT |
| | KDTYDALHMQALPPR |
| protease cleavable | GGGGAGPLIVGGGGT |
| linker 12 (SEQ ID NO: | |
| 1093) | |
| Construct C3559 (SEQ ID | MALPVTALLLPLALLLHAARPEQKLISEEDLEVQLVESGGGL |
| NO: 1094) | VQPGNSLRLSCAASGFTFSKFGMSWVRQRQSTFMAMDMPILE |
| | WVSSISGSGRDTLYADSVKGRFTISRDNAKTTLYLQMNSLRP |
| | EDTAVYYCTIGGSLSVSSQGTLVTVSSGGGGAGPLIVGGGGT |
| | EVQLLESGGGLVQPGGSLTLSCAASGFIFRAASMAWYRQSPG |
| | NERELVASISSGAFTNYADSVKARFTISRDNSKNTLYLQMNS |
| | LRAEDTAVYYCGATFLRSDGHHTINGQGTLVTVSSTSDYKDD |
| | DDKTTTPAPRPPTPAPTIASQPLSLRPEACRPAAGGAVHTRG |
| | LDFACDIYIWAPLAGTCGVLLLSLVITLYKRGRKKLLYIFKQ |
| | PFMRPVQTTQEEDGCSCRFPEEEEGGCELRVKFSRSADAPAY |
| | QQGQNQLYNELNLGRREEYDVLDKRRGRDPEMGGKPRRKNPQ |
| | EGLYNELQKDKMAEAYSEIGMKGERRRGKGHDGLYQGLSTAT |
| | KDTYDALHMQALPPR |
| protease cleavable | GGGGLRELHLGGGGT |
| linker 13 (SEQ ID NO: | |
| 1095) | |
| Construct C3560 (SEQ ID | MALPVTALLLPLALLLHAARPEQKLISEEDLEVQLVESGGGL |
| NO: 1096) | VQPGNSLRLSCAASGFTFSKFGMSWVRQRQSTFMAMDMPILE |
| | WVSSISGSGRDTLYADSVKGRFTISRDNAKTTLYLQMNSLRP |
| | EDTAVYYCTIGGSLSVSSQGTLVTVSSGGGGLRELHLGGGGT |
| | EVQLLESGGGLVQPGGSLTLSCAASGFIFRAASMAWYRQSPG |
| | NERELVASISSGAFTNYADSVKARFTISRDNSKNTLYLQMNS |
| | LRAEDTAVYYCGATFLRSDGHHTINGQGTLVTVSSTSDYKDD |
| | DDKTTTPAPRPPTPAPTIASQPLSLRPEACRPAAGGAVHTRG |
| | LDFACDIYIWAPLAGTCGVLLLSLVITLYKRGRKKLLYIFKQ |
| | PFMRPVQTTQEEDGCSCRFPEEEEGGCELRVKESRSADAPAY |
| | QQGQNQLYNELNLGRREEYDVLDKRRGRDPEMGGKPRRKNPQ |
| | EGLYNELQKDKMAEAYSEIGMKGERRRGKGHDGLYQGLSTAT |
| | KDTYDALHMQALPPR |
| Construct C2446 (SEQ ID | MALPVTALLLPLALLLHAARPEVQLVESGGGLVQPGNSLRLS |
| NO: 1097) | CAASGFTFSKFGMSWVRQANPWVPPREGLEWVSSISGSGRDT |
| | LYADSVKGRFTISRDNAKTTLYLQMNSLRPEDTAVYYCTIGG |
| | SLSVSSQGTLVTVSSGGGGPPASSGRAGGGGGGTEVQLLESG |
| | GGLVQPGGSLTLSCAASGRSFSNYIMGWFRQAPGKEREFVAG |
| | LGWSPGNTYYADSVKGRFTISRDNSKNTLYLQMNSLRAEDTA |
| | VYYCAARRGDVIYTTPWNYVYWGLGTQVTVSSTSDYKDDDDK |
| | TTTPAPRPPTPAPTIASQPLSLRPEACRPAAGGAVHTRGLDF |
| | ACDIYIWAPLAGTCGVLLLSLVITLYKRGRKKLLYIFKQPFM |
| | RPVQTTQEEDGCSCRFPEEEEGGCELRVKFSRSADAPAYQQG |
| | QNQLYNELNLGRREEYDVLDKRRGRDPEMGGKPRRKNPQEGL |
| | YNELQKDKMAEAYSEIGMKGERRRGKGHDGLYQGLSTATKDT |
| | YDALHMQALPPR |
| Construct C2510 (SEQ ID | MALPVTALLLPLALLLHAARPEVQLVESGGGLVQPGNSLRLS |
| NO: 1098) | CAASGFTFSKFGMSWVRQANPWVPPREGLEWVSSISGSGRDT |
| | LYADSVKGRFTISRDNAKTTLYLQMNSLRPEDTAVYYCTIGG |
| | SLSVSSQGTLVTVSSGGGGLSGRSDNHGGGGTEVQLLESGGG |
| | LVQPGGSLTLSCAASGRSFSNYIMGWFRQAPGKEREFVAGLG |
| | WSPGNTYYADSVKGRFTISRDNSKNTLYLQMNSLRAEDTAVY |
| | YCAARRGDVIYTTPWNYVYWGLGTQVTVSSTSDYKDDDDKTT |
| | TPAPRPPTPAPTIASQPLSLRPEACRPAAGGAVHTRGLDFAS |
| | DIYIWAPLAGTCGVLLLSLVITLYKRGRKKLLYIFKQPFMRP |
| | VQTTQEEDGCSCRFPEEEEGGCELRVKFSRSADAPAYQQGQN |
| | QLYNELNLGRREEYDVLDKRRGRDPEMGGKPRRKNPQEGLYN |
| | ELQKDKMAEAYSEIGMKGERRRGKGHDGLYQGLSTATKDTYD |
| | ALHMQALPPR |
| Construct C2513 (SEQ ID | MALPVTALLLPLALLLHAARPEVQLVESGGGLVQPGNSLRLS |
| NO: 1099) | CAASGFTFSKFGMSWVRQANPWVPPREGLEWVSSISGSGRDT |
| | LYADSVKGRFTISRDNAKTTLYLQMNSLRPEDTAVYYCTIGG |
| | SLSVSSQGTLVTVSSGGGGPPASSGRAGGGGGGTEVQLLESG |
| | GGLVQPGGSLTLSCAASGRSFSNYIMGWFRQAPGKEREFVAG |
| | LGWSPGNTYYADSVKGRFTISRDNSKNTLYLQMNSLRAEDTA |
| | VYYCAARRGDVIYTTPWNYVYWGLGTQVTVSSTSDYKDDDDK |
| | TTTPAPRPPTPAPTIASQPLSLRPEACRPAAGGAVHTRGLDF |
| | ASDIYIWAPLAGTCGVLLLSLVITLYKRGRKKLLYIFKQPFM |
| | RPVQTTQEEDGCSCRFPEEEEGGCELRVKFSRSADAPAYQQG |
| | QNQLYNELNLGRREEYDVLDKRRGRDPEMGGKPRRKNPQEGL |
| | YNELQKDKMAEAYSEIGMKGERRRGKGHDGLYQGLSTATKDT |
| | YDALHMQALPPR |
| Construct C2514 (SEQ ID | MALPVTALLLPLALLLHAARPEVQLVESGGGLVQPGNSLRLS |
| NO: 1100) | CAASGFTFSKFGMSWVRQANPWVPPREGLEWVSSISGSGRDT |
| | LYADSVKGRFTISRDNAKTTLYLQMNSLRPEDTAVYYCTIGG |
| | SLSVSSQGTLVTVSSGSGGSGGGGSGGGGGSGEVQLLESGGG |
| | LVQPGGSLTLSCAASGRSFSNYIMGWFRQAPGKEREFVAGLG |
| | WSPGNTYYADSVKGRFTISRDNSKNTLYLQMNSLRAEDTAVY |
| | YCAARRGDVIYTTPWNYVYWGLGTQVTVSSTSDYKDDDDKTT |
| | TPAPRPPTPAPTIASQPLSLRPEACRPAAGGAVHTRGLDFAS |
| | DIYIWAPLAGTCGVLLLSLVITLYKRGRKKLLYIFKQPFMRP |
| | VQTTQEEDGCSCRFPEEEEGGCELRVKFSRSADAPAYQQGQN |
| | QLYNELNLGRREEYDVLDKRRGRDPEMGGKPRRKNPQEGLYN |
| | ELQKDKMAEAYSEIGMKGERRRGKGHDGLYQGLSTATKDTYD |
| | ALHMQALPPR |
| Construct C2448 (SEQ ID | MALPVTALLLPLALLLHAARPEVQLLESGGGLVQPGGSLTLS |
| NO: 1101) | CAASGRSFSNYIMGWFRQAPGKEREFVAGLGWSPGNTYYADS |
| | VKGRFTISRDNSKNTLYLQMNSLRAEDTAVYYCAARRGDVIY |
| | TTPWNYVYWGLGTQVTVSSTSDYKDDDDKTTTPAPRPPTPAP |
| | TIASQPLSLRPEACRPAAGGAVHTRGLDFASDIYIWAPLAGT |
| | CGVLLLSLVITLYKRGRKKLLYIFKQPFMRPVQTTQEEDGCS |
| | CRFPEEEEGGCELRVKFSRSADAPAYQQGQNQLYNELNLGRR |
| | EEYDVLDKRRGRDPEMGGKPRRKNPQEGLYNELQKDKMAEAY |
| | SEIGMKGERRRGKGHDGLYQGLSTATKDTYDALHMQALPPR |
| Construct C3444 (SEQ ID | MALPVTALLLPLALLLHAARPEVQLVESGGGLVQPGNSLRLS |
| NO: 1102) | CAASGFTFSKFGMSWVRQRQSTFMAMDMPILEWVSSISGSGR |
| | DTLYADSVKGRFTISRDNAKTTLYLQMNSLRPEDTAVYYCTI |
| | GGSLSVSSQGTLVTVSSGGGGKPAEFTVDGGGGTEVQLLESG |
| | GGLVQPGGSLTLSCAASGFIFRAASMAWYRQSPGNERELVAS |
| | ISSGAFTNYADSVKARFTISRDNSKNTLYLQMNSLRAEDTAV |
| | YYCGATFLRSDGHHTINGQGTLVTVSSTSDYKDDDDKTTTPA |
| | PRPPTPAPTIASQPLSLRPEACRPAAGGAVHTRGLDFACDIY |
| | IWAPLAGTCGVLLLSLVITLYKRGRKKLLYIFKQPFMRPVQT |
| | TQEEDGCSCRFPEEEEGGCELRVKFSRSADAPAYQQGQNQLY |
| | NELNLGRREEYDVLDKRRGRDPEMGGKPRRKNPQEGLYNELQ |
| | KDKMAEAYSEIGMKGERRRGKGHDGLYQGLSTATKDTYDALH |
| | MQALPPR |
| Construct C3445 (SEQ ID | MALPVTALLLPLALLLHAARPEVQLVESGGGLVQPGNSLRLS |
| NO: 1103) | CAASGFTFSKFGMSWVRQRQSTFMAMDMPILEWVSSISGSGR |
| | DTLYADSVKGRFTISRDNAKTTLYLQMNSLRPEDTAVYYCTI |
| | GGSLSVSSQGTLVTVSSGGGGAISELFAKGGGGTEVQLLESG |
| | GGLVQPGGSLTLSCAASGFIFRAASMAWYRQSPGNERELVAS |
| | ISSGAFTNYADSVKARFTISRDNSKNTLYLQMNSLRAEDTAV |
| | YYCGATFLRSDGHHTINGQGTLVTVSSTSDYKDDDDKTTTPA |
| | PRPPTPAPTIASQPLSLRPEACRPAAGGAVHTRGLDFACDIY |
| | IWAPLAGTCGVLLLSLVITLYKRGRKKLLYIFKQPFMRPVQT |
| | TQEEDGCSCRFPEEEEGGCELRVKFSRSADAPAYQQGQNQLY |
| | NELNLGRREEYDVLDKRRGRDPEMGGKPRRKNPQEGLYNELQ |
| | KDKMAEAYSEIGMKGERRRGKGHDGLYQGLSTATKDTYDALH |
| | MQALPPR |
| Construct C1081 (SEQ ID | MALPVTALLLPLALLLHAARPTSDYKDDDDKTTTPAPRPPTP |
| NO: 1104) | APTIASQPLSLRPEACRPAAGGAVHTRGLDFACDIYIWAPLA |
| | GTCGVLLLSLVITLYKRGRKKLLYIFKQPFMRPVQTTQEEDG |
| | CSCRFPEEEEGGCELRVKFSRSADAPAYQQGQNQLYNELNLG |
| | RREEYDVLDKRRGRDPEMGGKPRRKNPQEGLYNELQKDKMAE |
| | AYSEIGMKGERRRGKGHDGLYQGLSTATKDTYDALHMQALPP |
| | R |
| Construct C1824 (SEQ ID | MALPVTALLLPLALLLHAARPEVQLLESGGGLVQPGGSLTLS |
| NO: 1105) | CAASGRSFSNYIMGWFRQAPGKEREFVAGLGWSPGNTYYADS |
| | VKGRFTISRDNSKNTLYLQMNSLRAEDTAVYYCAARRGDVIY |
| | TTPWNYVYWGLGTQVTVSSTSDYKDDDDKTTTPAPRPPTPAP |
| | TIASQPLSLRPEACRPAAGGAVHTRGLDFACDIYIWAPLAGT |
| | CGVLLLSLVITLYKRGRKKLLYIFKQPFMRPVQTTQEEDGCS |
| | CRFPEEEEGGCELRVKFSRSADAPAYQQGQNQLYNELNLGRR |
| | EEYDVLDKRRGRDPEMGGKPRRKNPQEGLYNELQKDKMAEAY |
| | SEIGMKGERRRGKGHDGLYQGLSTATKDTYDALHMQALPPR |
| Construct C1950 (SEQ ID | MALPVTALLLPLALLLHAARPEVQLVESGGGLVQPGNSLRLS |
| NO: 1106) | CAASGFTFSKFGMSWVRQANPWVPPREGLEWVSSISGSGRDT |
| | LYADSVKGRFTISRDNAKTTLYLQMNSLRPEDTAVYYCTIGG |
| | SLSVSSQGTLVTVSSGSGGSGGGGSGGGGGSGEVQLLESGGG |
| | LVQPGGSLTLSCAASGRSFSNYIMGWFRQAPGKEREFVAGLG |
| | WSPGNTYYADSVKGRFTISRDNSKNTLYLQMNSLRAEDTAVY |
| | YCAARRGDVIYTTPWNYVYWGLGTQVTVSSTSDYKDDDDKTT |
| | TPAPRPPTPAPTIASQPLSLRPEACRPAAGGAVHTRGLDFAC |
| | DIYIWAPLAGTCGVLLLSLVITLYKRGRKKLLYIFKQPFMRP |
| | VQTTQEEDGCSCRFPEEEEGGCELRVKFSRSADAPAYQQGQN |
| | QLYNELNLGRREEYDVLDKRRGRDPEMGGKPRRKNPQEGLYN |
| | ELQKDKMAEAYSEIGMKGERRRGKGHDGLYQGLSTATKDTYD |
| | ALHMQALPPR |
| Construct C2031 (SEQ ID | MALPVTALLLPLALLLHAARPEVQLVESGGGLVQPGNSLRLS |
| NO: 1107) | CAASGFTFSKFGMSWVRQANPWVPPREGLEWVSSISGSGRDT |
| | LYADSVKGRFTISRDNAKTTLYLQMNSLRPEDTAVYYCTIGG |
| | SLSVSSQGTLVTVSSGGGGLSGRSDNHGGGGTEVQLLESGGG |
| | LVQPGGSLTLSCAASGRSFSNYIMGWFRQAPGKEREFVAGLG |
| | WSPGNTYYADSVKGRFTISRDNSKNTLYLQMNSLRAEDTAVY |
| | YCAARRGDVIYTTPWNYVYWGLGTQVTVSSTSDYKDDDDKTT |
| | TPAPRPPTPAPTIASQPLSLRPEACRPAAGGAVHTRGLDFAC |
| | DIYIWAPLAGTCGVLLLSLVITLYKRGRKKLLYIFKQPFMRP |
| | VQTTQEEDGCSCRFPEEEEGGCELRVKFSRSADAPAYQQGQN |
| | QLYNELNLGRREEYDVLDKRRGRDPEMGGKPRRKNPQEGLYN |
| | ELQKDKMAEAYSEIGMKGERRRGKGHDGLYQGLSTATKDTYD |
| | ALHMQALPPR |
| Protease substrate | EYSG↓LSDG |
| corresponding to | |
| protease cleavable | |
| linker 8 (SEQ ID NO: | |
| 1108) | |
| Protease substrate | KPAE↓FTVD |
| corresponding to | |
| protease cleavable | |
| linker 9 (SEQ ID NO: | |
| 1109) | |
| Protease substrate | AISE↓LFAK |
| corresponding to | |
| protease cleavable | |
| linker 10 (SEQ ID NO: | |
| 1110) | |
| Protease substrate | GDF↓LAE |
| corresponding to | |
| protease cleavable | |
| linker 11 (SEQ ID NO: | |
| 1111) | |
| Protease substrate | AGP↓LIV |
| corresponding to | |
| protease cleavable | |
| linker 12 (SEQ ID NO: | |
| 1112) | |
| Protease substrate | LRE↓LHL |
| corresponding to | |
| protease cleavable | |
| linker 77 (SEQ ID NO: | |
| 1113) | |
| Protease substrate (SEQ | PLG↓L |
| ID NO: 1114) | |
| Protease substrate (SEQ | PLN↓L |
| ID NO: 1115) | |
| Protease substrate (SEQ | PAG↓L |
| ID NO: 1116) | |
| Protease substrate (SEQ | PAN↓L |
| ID NO: 1117) | |
| H90.2 (SEQ ID NO: 1118) | EVQLLESGGGLVQPGGSLTLSCAASGFIFRAASMAWYRQSPG |
| | NERELVASISSGAFTNYADSVKARFTISRDNSKNTLYLQMNS |
| | LRAEDTAVYYCGATFLRSDGHHTIWGQGTLVTVSS |
| L276 (SEQ ID NO: 1119) | PQASTGGSGG |
| protease cleavable linker 77 | GGGGLRELHLGGGGT |
| (SEQ ID NO: 1095) | |
| Protease cleavage site (SEQ ID | GPLGIAGQ |
| NO: 1120) | |
| Protease cleavage site (SEQ ID | AVRWLLTA |
| NO: 1121) | |
| EpCAM H90 ProTriTAC with | EVQLVESGGGLVQPGNSLRLSCAASGFTFSKFGMS |
| protease cleavable linker 11 | WVRQGGGGGLDGNEEPGGLEWVSSISGSGRDTLY |
| (SEQ ID NO: 1122) | ADSVKGRFTISRDNAKTTLYLQMNSLRPEDTAVYY |
| | CTIGGSLSVSSQGTLVTVSSGGGGGDFLAEGGGGT |
| | QTVVTQEPSLTVSPGGTVTLTCASSTGAVTSGNYPN |
| | WVQQKPGQAPRGLIGGTKFLVPGTPARFSGSLLGG |
| | KAALTLSGVQPEDEAEYYCTLWYSNRWVFGGGTK |
| | LTVLGGGGSGGGGSGGGGSEVQLVESGGGLVQPG |
| | GSLKLSCAASGFTFNKYAINWVRQAPGKGLEWVA |
| | RIRSKYNNYATYYADQVKDRFTISRDDSKNTAYLQ |
| | MNNLKTEDTAVYYCVRHANFGNSYISYWAYWGQ |
| | GTLVTVSSGGGGSGGGSEVQLLESGGGLVQPGGSL |
| | TLSCAASGFIFRAASMAWYRQSPGNERELVASISSG |
| | AFTNYADSVKARFTISRDNSKNTLYLQMNSLRAED |
| | TAVYYCGATFLRSDGHHTINGQGTLVTVSSHHHHH |
| | H |
| EpCAM H90 ProTriTAC with | EVQLVESGGGLVQPGNSLRLSCAASGFTFSKFGMS |
| protease cleavable linker 12 | WVRQGGGGGLDGNEEPGGLEWVSSISGSGRDTLY |
| (SEQ ID NO: 1123) | ADSVKGRFTISRDNAKTTLYLQMNSLRPEDTAVYY |
| | CTIGGSLSVSSQGTLVTVSSGGGGAGPLIVGGGGTQ |
| | TVVTQEPSLTVSPGGTVTLTCASSTGAVTSGNYPN |
| | WVQQKPGQAPRGLIGGTKFLVPGTPARFSGSLLGG |
| | KAALTLSGVQPEDEAEYYCTLWYSNRWVFGGGTK |
| | LTVLGGGGSGGGGSGGGGSEVQLVESGGGLVQPG |
| | GSLKLSCAASGFTFNKYAINWVRQAPGKGLEWVA |
| | RIRSKYNNYATYYADQVKDRFTISRDDSKNTAYLQ |
| | MNNLKTEDTAVYYCVRHANFGNSYISYWAYWGQ |
| | GTLVTVSSGGGGSGGGSEVQLLESGGGLVQPGGSL |
| | TLSCAASGFIFRAASMAWYRQSPGNERELVASISSG |
| | AFTNYADSVKARFTISRDNSKNTLYLQMNSLRAED |
| | TAVYYCGATFLRSDGHHTINGQGTLVTVSSHHHHH |
| | H |
| EpCAM H90 ProTriTAC with | EVQLVESGGGLVQPGNSLRLSCAASGFTFSKFGMS |
| protease cleavable linker 77 | WVRQGGGGGLDGNEEPGGLEWVSSISGSGRDTLY |
| (SEQ ID NO: 1124) | ADSVKGRFTISRDNAKTTLYLQMNSLRPEDTAVYY |
| | CTIGGSLSVSSQGTLVTVSSGGGGLRELHLGGGGTQ |
| | TVVTQEPSLTVSPGGTVTLTCASSTGAVTSGNYPN |
| | WVQQKPGQAPRGLIGGTKFLVPGTPARFSGSLLGG |
| | KAALTLSGVQPEDEAEYYCTLWYSNRWVFGGGTK |
| | LTVLGGGGSGGGGSGGGGSEVQLVESGGGLVQPG |
| | GSLKLSCAASGFTFNKYAINWVRQAPGKGLEWVA |
| | RIRSKYNNYATYYADQVKDRFTISRDDSKNTAYLQ |
| | MNNLKTEDTAVYYCVRHANFGNSYISYWAYWGQ |
| | GTLVTVSSGGGGSGGGSEVQLLESGGGLVQPGGSL |
| | TLSCAASGFIFRAASMAWYRQSPGNERELVASISSG |
| | AFTNYADSVKARFTISRDNSKNTLYLQMNSLRAED |
| | TAVYYCGATFLRSDGHHTINGQGTLVTVSSHHHHH |
| | H |
| L276 full sequence v1 (SEQ ID | GGGGPQASTGGSGGGGGGT |
| NO: 1125) | |
| L276 full sequence v2 (SEQ ID | GGGGPQASTGGSGGGGGGS |
| NO: 1126) | |
| L077 full sequence v1 (SEQ ID | GGGGLRELHLGGGGS |
| NO: 1127) | |
| L077 full sequence v2 (SEQ ID | GGGGLRELHLGGGGT |
| NO: 1128) | |

1.-53. (canceled)

54. A conditionally active binding protein comprising:
a binding moiety (M) which comprises a non-CDR loop, a cleavable linker (L), and a first target antigen binding domain (T1) linking the binding moiety (M) with the first target antigen binding domain (T1),
wherein the binding moiety is capable of masking the binding of the first target antigen binding domain (T1) to its target,
wherein the cleavable linker comprises a protease cleavage site, and
wherein the cleavable linker comprises an amino acid sequence having at least 90% sequence identity to a sequence selected from the group consisting of SEQ ID NOs: 910-931, 1077, 1079, 1081, 1083, 1086, 1088-1089, 1091, 1093, 1095, 1119, and 1125-1128.

55. The conditionally active binding protein of claim 54, wherein the protease cleavage site is recognized by a serine protease, a cysteine protease, an aspartate protease, a threonine protease, a glutamic acid protease, a metalloproteinase (MMP), a gelatinase, or a asparagine peptide lyase, and wherein the cleavage site is not recognized by an endogenous protease.

56. The conditionally active binding protein of claim 55, wherein the endogenous protease is MMP9.

57. The conditionally active binding protein of claim 54, wherein the protease cleavage site comprises an amino acid sequence selected from the group consisting of SEQ ID NOs: 985-996, 1108-1117, and 1120-1121.

58. The conditionally active binding protein of claim 54, wherein the cleavable linker comprises an amino acid sequence selected from the group consisting of SEQ ID NOs: 910-931, 1077, 1079, 1081, 1083, 1086, 1088-1089, 1091, 1093, 1095, 1119, and 1125-1128.

59. The conditionally active binding protein of claim 58, wherein the cleavable linker comprises an amino acid sequence selected from the group consisting of SEQ ID NOs: 910-931, 1119, and 1125-1126.

60. The conditionally active binding protein of claim 54, further comprising a second target antigen binding domain (T2).

61. The conditionally active binding protein of claim 54, wherein the first target antigen binding domain (T1) or the second target antigen binding domain (T2) bind to a tumor antigen, an immune modulatory protein, or an immune cell.

62. The conditionally active binding protein of claim 61, wherein the immune cell is a T cell.

63. The conditionally active binding protein claim 61, wherein the first target antigen binding domain (T1) binds to CD3 and the second target antigen binding domain (T2) binds to the tumor antigen.

64. The conditionally active binding protein of claim 63, wherein the binding moiety (M), the cleavable linker (L), the first target antigen binding domain (T1), and the second target antigen binding domain (T2) are in one of the following configurations: in the order of N-terminus to C-terminus: M: L:T1:T2, M:L:T2:T1, T1:T2:L:M, and T2:T1:L:M.

65. The conditionally active binding protein of claim 54, wherein the non-CDR loop provides a binding site specific for binding of the binding moiety to the first target antigen binding domain (T1).

66. The conditionally active binding protein of claim 54, wherein the non-CDR loop comprises a CC′ loop of a camelid VHH domain, a human VH domain, a humanized VH domain, or a single domain antibody.

67. The conditionally active binding protein of claim 54, wherein the binding moiety further comprises complementarity determining regions (CDRs).

68. The conditionally active binding protein of claim 67, wherein the CDRs of the binding moiety provide a binding site specific for a bulk serum protein.

69. The conditionally active binding protein of claim 68, wherein the bulk serum protein is albumin.

70. The conditionally active binding protein of claim 54, wherein the binding moiety comprises a sequence selected from the group consisting of SEQ ID NOs: 259-301 and 795.

71. The conditionally active binding protein of claim 54, wherein the cleavable linker sequence lacks an arginine.

72. A conditionally active binding protein comprising a binding moiety (M) which comprises a non-CDR loop, a cleavable linker (L), and a first target antigen binding domain (T1), wherein the binding moiety is capable of masking the binding of the first target antigen binding domain to its target, wherein the cleavable linker comprises an amino acid sequence selected from the group consisting of: SEQ ID NOs: 910-931 and 985-996, or an amino acid sequence comprising one or more mutations in a sequence selected from the group consisting of SEQ ID NOs: 910-931 and 985-996.

73. A method of treating a disease in a subject in need thereof, comprising administering to the subject an effective amount of a conditionally active binding protein comprising:
a binding moiety (M) which comprises a non-CDR loop, a cleavable linker (L), and a first target antigen binding domain (T1) linking the binding moiety (M) with the first target antigen binding domain (T1),
wherein the binding moiety is capable of masking the binding of the first target antigen binding domain (T1) to its target,
wherein the cleavable linker comprises a protease cleavage site, and
wherein the cleavable linker comprises an amino acid sequence having at least 90% sequence identity to a sequence selected from the group consisting of SEQ ID NOs: 910-931, 1077, 1079, 1081, 1083, 1086, 1088-1089, 1091, 1093, 1095, 1119, and 1125-1128.

74. A cleavable linker comprising a sequence selected from the group consisting of: SEQ ID NOs: 915-917, 919, 920, 929-931, 1077, 1079, 1081, 1083, 1086, 1088, 1089, 1091, 1093, 1095, and 1119.


SEQUENCE LISTING (1243 sequences)

SEQ ID NO: 1 - protein, 8 aa, synthetic construct
KRALGLPG

SEQ ID NO: 2 - protein, 40 aa, synthetic construct
DEDEDEDEDEDEDEDERPLALWRSDRDRDRDRDRDRDRDR

SEQ ID NO: 3 - skipped

SEQ ID NO: 4 - protein, 5 aa, synthetic construct
LEATA

SEQ ID NO: 5 - protein, 10 aa, synthetic construct
GGAANLVRGG

SEQ ID NO: 6 - protein, 10 aa, synthetic construct
SGRIGFLRTA

SEQ ID NO: 7 - protein, 6 aa, synthetic construct
PLGLAG

SEQ ID NO: 8 - protein, 6 aa, synthetic construct
  VARIANT 6: note = Any amino acid
PLGLAX

SEQ ID NO: 9 - protein, 6 aa, synthetic construct
  SITE 5: note = Methyl alanine
PLGCAG

SEQ ID NO: 10 - protein, 8 aa, synthetic construct
ESPAYYTA

SEQ ID NO: 11 - protein, 6 aa, synthetic construct
RLQLKL

SEQ ID NO: 12 - protein, 7 aa, synthetic construct
RLQLKAC

SEQ ID NO: 13 - protein, 6 aa, synthetic construct
  SITE 3: note = Citrulline
  SITE 5: note = Hypofluorous acid modified tyrosine
EPXGYL

SEQ ID NO: 14 - protein, 5 aa, synthetic construct
SGRSA

SEQ ID NO: 15 - protein, 4 aa, synthetic construct
DAFK

SEQ ID NO: 16 - protein, 5 aa, synthetic construct
GGGRR

SEQ ID NO: 17 - protein, 4 aa, synthetic construct
GFLG

SEQ ID NO: 18 - protein, 4 aa, synthetic construct
ALAL

SEQ ID NO: 19-20 - skipped (2 entries)

SEQ ID NO: 21 - protein, 5 aa, synthetic construct
  SITE 4: note = Ethyl phenylalanine
PICFF

SEQ ID NO: 22 - protein, 8 aa, synthetic construct
GGPRGLPG

SEQ ID NO: 23 - protein, 6 aa, synthetic construct
HSSKLQ

SEQ ID NO: 24 - protein, 7 aa, synthetic construct
HSSKLQL

SEQ ID NO: 25 - protein, 9 aa, synthetic construct
HSSKLQEDA

SEQ ID NO: 26 - protein, 10 aa, synthetic construct
LVLASSSFGY

SEQ ID NO: 27 - protein, 10 aa, synthetic construct
GVSQNYPIVG

SEQ ID NO: 28 - protein, 10 aa, synthetic construct
GVVQASCRLA

SEQ ID NO: 29 - skipped

SEQ ID NO: 30 - protein, 6 aa, synthetic construct
DPRSFL

SEQ ID NO: 31 - protein, 6 aa, synthetic construct
PPRSFL

SEQ ID NO: 32 - protein, 4 aa, synthetic construct
DEVD

SEQ ID NO: 33 - protein, 5 aa, synthetic construct
DEVDP

SEQ ID NO: 34 - protein, 8 aa, synthetic construct
KGSGDVEG

SEQ ID NO: 35 - protein, 6 aa, synthetic construct
GWEHDG

SEQ ID NO: 36 - protein, 7 aa, synthetic construct
EDDDDKA

SEQ ID NO: 37 - protein, 9 aa, synthetic construct
KQEQNPGST

SEQ ID NO: 38 - protein, 6 aa, synthetic construct
GKAFRR

SEQ ID NO: 39 - protein, 4 aa, synthetic construct
DAFK

SEQ ID NO: 40 - protein, 4 aa, synthetic construct
DVLK

SEQ ID NO: 41 - protein, 4 aa, synthetic construct
DAFK

SEQ ID NO: 42 - protein, 7 aa, synthetic construct
ALLLALL

SEQ ID NO: 43 - protein, 518 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSSHHHHHH

SEQ ID NO: 44 - protein, 518 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSSHHHHHH

SEQ ID NO: 45 - protein, 382 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSSHHHHHH

SEQ ID NO: 46 - protein, 534 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASGRTFSSYAMGWFRQAPGKEREFVVAINWASGSTYYADSVKGRFTISRDNSKNTLYLQMNSLRAEDTAVYYCAAGYQINSGNYNFKDYEYDYWGQGTLVTVSSHHHHHH

SEQ ID NO: 47 - protein, 534 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASGRTFSSYAMGWFRQAPGKEREFVVAINWASGSTYYADSVKGRFTISRDNSKNTLYLQMNSLRAEDTAVYYCAAGYQINSGNYNFKDYEYDYWGQGTLVTVSSHHHHHH

SEQ ID NO: 48 - protein, 398 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASGRTFSSYAMGWFRQAPGKEREFVVAINWASGSTYYADSVKGRFTISRDNSKNTLYLQMNSLRAEDTAVYYCAAGYQINSGNYNFKDYEYDYWGQGTLVTVSSHHHHHH

SEQ ID NO: 49 - protein, 503 aa, synthetic construct
QVQLVESGGALVQPGGSLRLSCAASGFPVNRYSMRWYRQAPGKEREWVAGMSSAGDRSSYEDSVKGRFTISRDDARNTVYLQMNSLKPEDTAVYYCNVNVGFEYWGQGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 50 - protein, 14 aa, synthetic construct
GGGGGLDGNEEPGG

SEQ ID NO: 51 - protein, 19 aa, synthetic construct
GGGGSGGGGSGGVVGGGGT

SEQ ID NO: 52 - protein, 509 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSSHHHHHH

SEQ ID NO: 53 - protein, 19 aa, synthetic construct
GGGGKPLGLQARVVGGGGT

SEQ ID NO: 54 - protein, 143 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGT

SEQ ID NO: 55 - protein, 127 aa, synthetic construct
EVQLVESGGGLVQPGGSLTLSCAASGRTFSSYAMGWFRQAPGKEREFVVAINWASGSTYYADSVKGRFTISRDNSKNTLYLQMNSLRAEDTAVYYCAAGYQINSGNYNFKDYEYDYWGQGTLVTVSS

SEQ ID NO: 56 - protein, 249 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 57 - protein, 111 aa, synthetic construct
EVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTVSS

SEQ ID NO: 58 - protein, 10 aa, synthetic construct
KPLGLQARVV

SEQ ID NO: 59 - protein, 10 aa, synthetic construct
PQASTGRSGG

SEQ ID NO: 60 - protein, 10 aa, synthetic construct
PQGSTGRAAG

SEQ ID NO: 61 - protein, 10 aa, synthetic construct
PPASSGRAGG

SEQ ID NO: 62 - protein, 9 aa, synthetic construct
PIPVQGRAH

SEQ ID NO: 63 - protein, 111 aa, synthetic construct
EVQLVESGGGLVQPGGSLRLSCAASRFMISEYHMHWVRQAPGKGLEWVSDINPAGTTDYAESVKGRFTISRDNAKNTLYLQMNSLRAEDTAVYYCDSYGYRGQGTLVTVSS

SEQ ID NO: 64 - protein, 111 aa, synthetic construct
EVQLVESGGGLVQPGGSLRLSCAASRFMISEYHMHWVRQAPGKGLEWVSTINPAGTTDYAESVKGRFTISRDNAKNTLYLQMNSLRAEDTAVYYCDSYGYRGQGTLVTVSS

SEQ ID NO: 65 - protein, 111 aa, synthetic construct
EVQLVESGGGLVQPGGSLRLSCAASRFMISEYSMHWVRQAPGKGLEWVSTINPAKTTDYAESVKGRFTISRDNAKNTLYLQMNSLRAEDTAVYYCDSYGYRGQGTLVTVSS

SEQ ID NO: 66 - protein, 111 aa, synthetic construct
EVQLVESGGGLVQPGGSLRLSCAASRFMISPYSMHWVRQAPGKGLEWVSTINPAGTTDYAESVKGRFTISRDNAKNTLYLQMNSLRAEDTAVYYCDGYGYRGQGTLVTVSS

SEQ ID NO: 67 - protein, 111 aa, synthetic construct
EVQLVESGGGLVQPGGSLRLSCAASRFMISEYSMHWVRQAPGKGLEWVSTINPAGQTDYAESVKGRFTISRDNAKNTLYLQMNSLRAEDTAVYYCDGYGYRGQGTLVTVSS

SEQ ID NO: 68 - protein, 111 aa, synthetic construct
EVQLVESGGGLVQPGGSLRLSCAASRFMISEYSMHWVRQAPGKGLEWVSTINPAGTTDYAEYVKGRFTISRDNAKNTLYLQMNSLRAEDTAVYYCDGYGYRGQGTLVTVSS

SEQ ID NO: 69 - protein, 111 aa, synthetic construct
EVQLVESGGGLVQPGGSLRLSCAASRFMISEYHMHWVRQAPGKGLEWVSDINPAKTTDYAESVKGRFTISRDNAKNTLYLQMNSLRAEDTAVYYCDSYGYRGQGTLVTVSS

SEQ ID NO: 70 - protein, 111 aa, synthetic construct
EVQLVESGGGLVQPGGSLRLSCAASRFMISPYHMHWVRQAPGKGLEWVSDINPAGTTDYAESVKGRFTISRDNAKNTLYLQMNSLRAEDTAVYYCDSYGYRGQGTLVTVSS

SEQ ID NO: 71 - protein, 111 aa, synthetic construct
EVQLVESGGGLVQPGGSLRLSCAASRFMISEYHMHWVRQAPGKGLEWVSDINPAGQTDYAESVKGRFTISRDNAKNTLYLQMNSLRAEDTAVYYCDSYGYRGQGTLVTVSS

SEQ ID NO: 72 - protein, 111 aa, synthetic construct
EVQLVESGGGLVQPGGSLRLSCAASRFMISEYHMHWVRQAPGKGLEWVSDINPAGTTDYAEYVKGRFTISRDNAKNTLYLQMNSLRAEDTAVYYCDSYGYRGQGTLVTVSS

SEQ ID NO: 73 - protein, 111 aa, synthetic construct
EVQLVESGGGLVQPGGSLTLSCAASRFMISEYHMHWVRQAPGKGLEWVSDINPAGTTDYAESVKGRFTISRDNAKNTLYLQMNSLKPEDTAVYYCDSYGYRGQGTQVTVSS

SEQ ID NO: 74 - protein, 249 aa, synthetic construct
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 75 - protein, 249 aa, synthetic construct
EVQLVESGGGLVQPGGSLKLSCAASGFEFNKYAMNWVRQAPGKGLEWVARIRSKYNKYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSFGAVTSGNYPNWVQQKPGQAPRGLIGGTKFLAPGTPARFSGSLLGGKAALTLSGVQPEDEAEYYCVLWYDNRWVFGGGTKLTVL

SEQ ID NO: 76 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSHISYWAYWGQGTLVTVSSGGGGSGGGGSGGGGSQTVVTQEPSLTVSPGGTVTLTCGSSTGYVTSGNYPNWVQQKPGQAPRGLIGGTSFLAPGTPARFSGSLLGGKAALTLSGVQPEDEAEYYCVLWYSNRWIFGGGTKLTVL

SEQ ID NO: 77 - protein, 249 aa, synthetic construct
EVQLVESGGGLVQPGGSLKLSCAASGFMFNKYAMNWVRQAPGKGLEWVARIRSKSNNYATYYADSVKDRFTISRDDSKNTAYLQMNNLKTEDTAVYYCVRHGNFGNSYISYWATWGQGTLVTVSSGGGGSGGGGSGGGGSQTVVTQEPSLTVSPGGTVTLTCGSSFGAVTSGNYPNWVQQKPGQAPRGLIGGTKLLAPGTPARFSGSLLGGKAALTLSGVQPEDEAEYYCVLWYSNSWVFGGGTKLTVL

SEQ ID NO: 78 - protein, 249 aa, synthetic construct
EVQLVESGGGLVQPGGSLKLSCAASGFTFNTYAMNWVRQAPGKGLEWVARIRSKYNNYATYYKDSVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSTGAVVSGNYPNWVQQKPGQAPRGLIGGTEFLAPGTPARFSGSLLGGKAALTLSGVQPEDEAEYYCVLWYSNRWVFGGGTKLTVL

SEQ ID NO: 79 - protein, 249 aa, synthetic construct
EVQLVESGGGLVQPGGSLKLSCAASGFTYNKYAMNWVRQAPGKGLEWVARIRSKYNNYATYYADEVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSKGAVTSGNYPNWVQQKPGQAPRGLIGGTKELAPGTPARFSGSLLGGKAALTLSGVQPEDEAEYYCTLWYSNRWVFGGGTKLTVL

SEQ ID NO: 80 - protein, 249 aa, synthetic construct
EVQLVESGGGLVQPGGSLKLSCAASGNTFNKYAMNWVRQAPGKGLEWVARIRSKYNNYETYYADSVKDRFTISRDDSKNTAYLQMNNLKTEDTAVYYCVRHTNFGNSYISYWAYWGQGTLVTVSSGGGGSGGGGSGGGGSQTVVTQEPSLTVSPGGTVTLTCGSSTGAVTSGYYPNWVQQKPGQAPRGLIGGTYFLAPGTPARFSGSLLGGKAALTLSGVQPEDEAEYYCVLWYSNRWVFGGGTKLTVL

SEQ ID NO: 81 - protein, 249 aa, synthetic construct
EVQLVESGGGLVQPGGSLKLSCAASGFTFNNYAMNWVRQAPGKGLEWVARIRSKYNNYATYYADAVKDRFTISRDDSKNTAYLQMNNLKTEDTAVYYCVRHGNFGNSQISYWAYWGQGTLVTVSSGGGGSGGGGSGGGGSQTVVTQEPSLTVSPGGTVTLTCGSSTGAVTDGNYPNWVQQKPGQAPRGLIGGIKFLAPGTPARFSGSLLGGKAALTLSGVQPEDEAEYYCVLWYSNRWVFGGGTKLTVL

SEQ ID NO: 82 - protein, 249 aa, synthetic construct
EVQLVESGGGLVQPGGSLKLSCAASGFTFNKYAVNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGESTGAVTSGNYPNWVQQKPGQAPRGLIGGTKILAPGTPARFSGSLLGGKAALTLSGVQPEDEAEYYCVLWYSNRWVFGGGTKLTVL

SEQ ID NO: 83 - protein, 249 aa, synthetic construct
EVQLVESGGGLVQPGGSLKLSCAASGFTFNKYPMNWVRQAPGKGLEWVARIRSKYNNYATYYADSVKDRFTISRDDSKNTAYLQMNNLKNEDTAVYYCVRHGNFNNSYISYWAYWGQGTLVTVSSGGGGSGGGGSGGGGSQTVVTQEPSLTVSPGGTVTLTCGSSTGAVTKGNYPNWVQQKPGQAPRGLIGGTKMLAPGTPARFSGSLLGGKAALTLSGVQPEDEAEYYCALWYSNRWVFGGGTKLTVL

SEQ ID NO: 84 - protein, 249 aa, synthetic construct
EVQLVESGGGLVQPGGSLKLSCAASGFTFNGYAMNWVRQAPGKGLEWVARIRSKYNNYATYYADEVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSTGAVVSGNYPNWVQQKPGQAPRGLIGGTEFLAPGTPARFSGSLLGGKAALTLSGVQPEDEAEYYCVLWYSNRWVFGGGTKLTVL

SEQ ID NO: 85 - protein, 249 aa, synthetic construct
EVQLVESGGGLVQPGGSLKLSCAASGNTFNKYAMNWVRQAPGKGLEWVARIRSKYNNYATYYADSVKDRFTISRDDSKNTAYLQMNNLKTEDTAVYYCVRHGNFGDSYISYWAYWGQGTLVTVSSGGGGSGGGGSGGGGSQTVVTQEPSLTVSPGGTVTLTCGSSTGAVTHGNYPNWVQQKPGQAPRGLIGGTKVLAPGTPARFSGSLLGGKAALTLSGVQPEDEAEYYCVLWYSNRWVFGGGTKLTVL

SEQ ID NO: 86 - protein, 249 aa, synthetic construct
EVQLVESGGGLVQPGGSLKLSCAASGFTFNNYAMNWVRQAPGKGLEWVARIRSGYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSYTGAVTSGNYPNWVQQKPGQAPRGLIGGTKFNAPGTPARFSGSLLGGKAALTLSGVQPEDEAEYYCVLWYANRWVFGGGTKLTVL

SEQ ID NO: 87 - protein, 249 aa, synthetic construct
EVQLVESGGGLVQPGGSLKLSCAASGFEFNKYAMNWVRQAPGKGLEWVARIRSKYNNYETYYADSVKDRFTISRDDSKNTAYLQMNNLKTEDTAVYYCVRHGNFGNSLISYWAYWGQGTLVTVSSGGGGSGGGGSGGGGSQTVVTQEPSLTVSPGGTVTLTCGSSSGAVTSGNYPNWVQQKPGQAPRGLIGGTKFGAPGTPARFSGSLLGGKAALTLSGVQPEDEAEYYCVLWYSNRWVFGGGTKLTVL

SEQ ID NO: 88 - protein, 249 aa, synthetic construct
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 89 - protein, 249 aa, synthetic construct
EVQLVESGGGLVQPGGSLKLSCAASGFTFNKYALNWVRQAPGKGLEWVARIRSKYNNYATEYADSVKDRFTISRDDSKNTAYLQMNNLKTEDTAVYYCVRHGNFGNSPISYWAYWGQGTLVTVSSGGGGSGGGGSGGGGSQTVVTQEPSLTVSPGGTVTLTCGSSTGAVTSGNYPNWVQQKPGQAPRGLIGGTNFLAPGTPERFSGSLLGGKAALTLSGVQPEDEAEYYCVLWYSNRWAFGGGTKLTVL

SEQ ID NO: 90 - protein, 249 aa, synthetic construct
EVQLVESGGGLVQPGGSLKLSCAASGFTFNEYAMNWVRQAPGKGLEWVARIRSKYNNYATYYADDVKDRFTISRDDSKNTAYLQMNNLKTEDTAVYYCVRHGNFGNSGISYWAYWGQGTLVTVSSGGGGSGGGGSGGGGSQTVVTQEPSLTVSPGGTVTLTCGSSTGAVTVGNYPNWVQQKPGQAPRGLIGGTEFLAPGTPARFSGSLLGGKAALTLSGVQPEDEAEYYCVLWYSNRWVFGGGTKLTVL

SEQ ID NO: 91 - protein, 119 aa, synthetic construct
EVQLVESGGGLVQPGRSLTLSCAASTNIFSISPMGWYRQAPGKQRELVAAIHGFSTLYADSVKGRFTISRDNAKNSIYLQMNSLRPEDTALYYCNKVPWGDYHPRNVYWGQGTQVTVSS

SEQ ID NO: 92 - protein, 119 aa, synthetic construct
EVQLVESGGGLVQPGRSLTLSCAASTDIFSISPMGWYRQAPGKQRELVAAIHGGSTLYADSVKGRFTISRDNAKNSIYLQMNSLRPEDTALYYCNKVPWGDYHPRNVAWGQGTQVTVSS

SEQ ID NO: 93 - protein, 119 aa, synthetic construct
EVQLVESGGGLVQPGRSLTLSCAASTNDFSISPMGWYRQAPGKQRELVAAIHGGSTLYADSVKGRFTISRDNAKNSIYLQMNSLRPEDTALYYCNKVPWGDYHPRNVAWGQGTQVTVSS

SEQ ID NO: 94 - protein, 119 aa, synthetic construct
EVQLVESGGGLVQPGRSLTLSCAASTNIFSKSPMGWYRQAPGKQRELVAAIHGKSTLYADSVKGRFTISRDNAKNSIYLQMNSLRPEDTALYYCNKVPWGDYHPRNVVWGQGTQVTVSS

SEQ ID NO: 95 - protein, 119 aa, synthetic construct
EVQLVESGGGLVQPGRSLTLSCAASTNDFSISPMGWYRQAPGKQRELVAAIHGKSTLYADSVKGRFTISRDNAKNSIYLQMNSLRPEDTALYYCNKVPWGDYHPRNVKWGQGTQVTVSS

SEQ ID NO: 96 - protein, 119 aa, synthetic construct
EVQLVESGGGLVQPGRSLTLSCAASTNQFSISPMGWYRQAPGKQRELVAAIHGKSTLYADSVKGRFTISRDNAKNSIYLQMNSLRPEDTALYYCNKVPWGDYHPRNVVWGQGTQVTVSS

SEQ ID NO: 97 - protein, 119 aa, synthetic construct
EVQLVESGGGLVQPGRSLTLSCAASTNIFSSSPMGWYRQAPGKQRELVAAINGFSTLYADSVKGRFTISRDNAKNSIYLQMNSLRPEDTALYYCNKVPWGDYHPRNVHWGQGTQVTVSS

SEQ ID NO: 98 - protein, 119 aa, synthetic construct
EVQLVESGGGLVQPGRSLTLSCAASTNIFSSSPMGWYRQAPGKQRELVAAIHGFSTLYADSVKGRFTISRDNAKNSIYLQMNSLRPEDTALYYCNKVPWGDYHPRNVYWGQGTQVTVSS

SEQ ID NO: 99 - protein, 119 aa, synthetic construct
EVQLVESGGGLVQPGRSLTLSCAASTNIFSTSPMGWYRQAPGKQRELVAAIHGFQTLYADSVKGRFTISRDNAKNSIYLQMNSLRPEDTALYYCNKVPWGDYHPRNVVWGQGTQVTVSS

SEQ ID NO: 100 - protein, 119 aa, synthetic construct
EVQLVESGGGLVQPGRSLTLSCAASTNIFSRSPMGWYRQAPGKQRELVAAIHGFETLYADSVKGRFTISRDNAKNSIYLQMNSLRPEDTALYYCNKVPWGDYHPRNVLWGQGTQVTVSS

SEQ ID NO: 101 - protein, 119 aa, synthetic construct
EVQLVESGGGLVQPGRSLTLSCAASTNIFSESPMGWYRQAPGKQRELVAAIHGFTTLYADSVKGRFTISRDNAKNSIYLQMNSLRPEDTALYYCNKVPWGDYHPRNVTWGQGTQVTVSS

SEQ ID NO: 102 - protein, 119 aa, synthetic construct
EVQLVESGGGLVQPGRSLTLSCAASTNIFSDSPMGWYRQAPGKQRELVAAIHGFSTLYADSVKGRFTISRDNAKNSIYLQMNSLRPEDTALYYCNKVPWGDYHPRNVAWGQGTQVTVSS

SEQ ID NO: 103 - protein, 119 aa, synthetic construct
EVQLVESGGGLVQPGRSLTLSCAASTNIFSNSPMGWYRQAPGKQRELVAAIHGGSTLYADSVKGRFTISRDNAKNSIYLQMNSLRPEDTALYYCNKVPWGDYHPRNVHWGQGTQVTVSS

SEQ ID NO: 104 - protein, 119 aa, synthetic construct
EVQLVESGGGLVQPGRSLTLSCAASTNIFSNSPMGWYRQAPGKQRELVAAIHGRSTLYADSVKGRFTISRDNAKNSIYLQMNSLRPEDTALYYCNKVPWGDYHPRNVMWGQGTQVTVSS

SEQ ID NO: 105 - protein, 119 aa, synthetic construct
EVQLVESGGGLVQPGRSLTLSCAASTNIFSNSPMGWYRQAPGKQRELVAAIHGPSTLYADSVKGRFTISRDNAKNSIYLQMNSLRPEDTALYYCNKVPWGDYHPRNVTWGQGTQVTVSS

SEQ ID NO: 106 - protein, 119 aa, synthetic construct
EVQLVESGGGLVQPGRSLTLSCAASTNIFSNSPMGWYRQAPGKQRELVAAIHGDSTLYADSVKGRFTISRDNAKNSIYLQMNSLRPEDTALYYCNKVPWGDYHPRNVRWGQGTQVTVSS

SEQ ID NO: 107 - protein, 119 aa, synthetic construct
EVQLVESGGGLVQPGRSLTLSCAASTNIFSRSPMGWYRQAPGKQRELVAAIHGDSTLYADSVKGRFTISRDNAKNSIYLQMNSLRPEDTALYYCNKVPWGDYHPRNVTWGQGTQVTVSS

SEQ ID NO: 108 - protein, 119 aa, synthetic construct
EVQLVESGGGLVQPGRSLTLSCAASTNIFSKSPMGWYRQAPGKQRELVAAIHGQSTLYADSVKGRFTISRDNAKNSIYLQMNSLRPEDTALYYCNKVPWGDYHPRNVTWGQGTQVTVSS

SEQ ID NO: 109 - protein, 119 aa, synthetic construct
EVQLVESGGGLVQPGRSLTLSCAASTNIFSSSPMGWYRQAPGKQRELVAAIHGHSTLYADSVKGRFTISRDNAKNSIYLQMNSLRPEDTALYYCNKVPWGDYHPRNVTWGQGTQVTVSS

SEQ ID NO: 110 - protein, 119 aa, synthetic construct
EVQLVESGGGLVQPGRSLTLSCAASTNIFSSSPMGWYRQAPGKQRELVAAIHGESTLYADSVKGRFTISRDNAKNSIYLQMNSLRPEDTALYYCNKVPWGDYHPRKVYWGQGTQVTVSS

SEQ ID NO: 111 - protein, 119 aa, synthetic construct
EVQLVESGGGLVQPGRSLTLSCAASTNIFSRSPMGWYRQAPGKQRELVAAIHGNSTLYADSVKGRFTISRDNAKNSIYLQMNSLRPEDTALYYCNKVPWGIYHPRNVYWGQGTQVTVSS

SEQ ID NO: 112 - protein, 119 aa, synthetic construct
EVQLVESGGGLVQPGRSLTLSCAASTNIFSESPMGWYRQAPGKQRELVAAIHGNSTLYADSVKGRFTISRDNAKNSIYLQMNSLRPEDTALYYCNKVPWGTYHPRNVYWGQGTQVTVSS

SEQ ID NO: 113 - protein, 119 aa, synthetic construct
EVQLVESGGGLVQPGRSLTLSCAASTNIFSVSPMGWYRQAPGKQRELVAAIHGNSTLYADSVKGRFTISRDNAKNSIYLQMNSLRPEDTALYYCNKVPWGKYHPRNVYWGQGTQVTVSS

SEQ ID NO: 114 - protein, 119 aa, synthetic construct
EVQLVESGGGLVQPGRSLTLSCAASTNIFSVSPMGWYRQAPGKQRELVAAIHGNSTLYADSVKGRFTISRDNAKNSIYLQMNSLRPEDTALYYCNKVPWGDYHPRNVYWGQGTQVTVSS

SEQ ID NO: 115 - protein, 119 aa, synthetic construct
EVQLVESGGGLVQPGRSLTLSCAASTNIFSKSPMGWYRQAPGKQRELVAAIHGNSTLYADSVKGRFTISRDNAKNSIYLQMNSLRPEDTALYYCNKVPWGDYHPREVYWGQGTQVTVSS

SEQ ID NO: 116 - protein, 119 aa, synthetic construct
EVQLVESGGGLVQPGRSLTLSCAASTNIFSDSPMGWYRQAPGKQRELVAAIHGTSTLYADSVKGRFTISRDNAKNSIYLQMNSLRPEDTALYYCNKVPWGDYHPRNVYWGQGTQVTVSS

SEQ ID NO: 117 - protein, 119 aa, synthetic construct
EVQLVESGGGLVQPGRSLTLSCAASTNIFSRSPMGWYRQAPGKQRELVAAIHGTSTLYADSVKGRFTISRDNAKNSIYLQMNSLRPEDTALYYCNKVPWGKYHPRNVYWGQGTQVTVSS

SEQ ID NO: 118 - protein, 119 aa, synthetic construct
EVQLVESGGGLVQPGRSLTLSCAASTNIFSHSPMGWYRQAPGKQRELVAAIHGTSTLYADSVKGRFTISRDNAKNSIYLQMNSLRPEDTALYYCNKVPWGRYHPRNVYWGQGTQVTVSS

SEQ ID NO: 119 - protein, 119 aa, synthetic construct
EVQLVESGGGLVQPGRSLTLSCAASTNIFSTSPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 120 - protein, 119 aa, synthetic construct
EVQLVESGGGLVQPGRSLTLSCAASTNIFSTSPMGWYRQAPGKQRELVAAIHGTSTLYADSVKGRFTISRDNAKNSIYLQMNSLRPEDTALYYCNKVPWGDYHPRNVQWGQGTQVTVSS

SEQ ID NO: 121 - protein, 119 aa, synthetic construct
EVQLVESGGGLVQPGRSLTLSCAASTNIFSLSPMGWYRQAPGKQRELVAAIHGDSTLYADSVKGRFTISRDNAKNSIYLQMNSLRPEDTALYYCNKVPWGDYHPRNVYWGQGTQVTVSS

SEQ ID NO: 122 - protein, 119 aa, synthetic construct
EVQLVESGGGLVQPGRSLTLSCAASTNIFSISPGGWYRQAPGKQRELVAAIHGSSTLYADSVKGRFTISRDNAKNSIYLQMNSLRPEDTALYYCNKVPWGDYHPRNVYWGQGTQVTVSS

SEQ ID NO: 123 - protein, 119 aa, synthetic construct
EVQLVESGGGLVQPGRSLTLSCAASTNHFSISPMGWYRQAPGKQRELVAAIHGSSTLYADSVKGRFTISRDNAKNSIYLQMNSLRPEDTALYYCNKVPWGDYHPRVVYWGQGTQVTVSS

SEQ ID NO: 124 - protein, 119 aa, synthetic construct
EVQLVESGGGLVQPGRSLTLSCAASTNIFSASPMGWYRQAPGKQRELVAAIHGSSTLYADSVKGRFTISRDNAKNSIYLQMNSLRPEDTALYYCNKVPWGDYHPRNVNWGQGTQVTVSS

SEQ ID NO: 125 - protein, 119 aa, synthetic construct
EVQLVESGGGLVQPGRSLTLSCAASTNIFSASPMGWYRQAPGKQRELVAAIHGSSTLYADSVKGRFTISRDNAKNSIYLQMNSLRPEDTALYYCNKVPWGRYHPRNVYWGQGTQVTVSS

SEQ ID NO: 126 - protein, 119 aa, synthetic construct
EVQLVESGGGLVQPGRSLTLSCAASTNISSISPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 127 - protein, 119 aa, synthetic construct
EVQLVESGGGLVQPGRSLTLSCAASTNIESISPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 128 - protein, 119 aa, synthetic construct
EVQLVESGGGLVQPGRSLTLSCAASTNIFSISPYGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 129 - protein, 119 aa, synthetic construct
EVQLVESGGGLVQPGRSLTLSCAASTNIASISPMGWYRQAPGKQRELVAAIHGTSTLYADSVKGRFTISRDNAKNSIYLQMNSLRPEDTALYYCNKVPWGDYHPGNVYWGQGTQVTVSS

SEQ ID NO: 130 - protein, 119 aa, synthetic construct
EVQLVESGGGLVQPGRSLTLSCAASTNIASISPMGWYRQAPGKQRELVAAIHGKSTLYADSVKGRFTISRDNAKNSIYLQMNSLRPEDTALYYCNKVPWGDYHPGNVYWGQGTQVTVSS

SEQ ID NO: 131 - protein, 119 aa, synthetic construct
EVQLVESGGGLVQPGRSLTLSCAASTNIASISPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 132 - protein, 119 aa, synthetic construct
EVQLVESGGGLVQPGRSLTLSCAASTNITSISPMGWYRQAPGKQRELVAAIHGDSTLYADSVKGRFTISRDNAKNSIYLQMNSLRPEDTALYYCNKVPWGDYHPGNVYWGQGTQVTVSS

SEQ ID NO: 133 - protein, 119 aa, synthetic construct
EVQLVESGGGLVQPGRSLTLSCAASTNIMSISPMGWYRQAPGKQRELVAAIHGNSTLYADSVKGRFTISRDNAKNSIYLQMNSLRPEDTALYYCNKVPWGDYHPGNVYWGQGTQVTVSS

SEQ ID NO: 134 - protein, 119 aa, synthetic construct
EVQLVESGGGLVQPGRSLTLSCAASTNITSISPMGWYRQAPGKQRELVAAIHGNSTLYADSVKGRFTISRDNAKNSIYLQMNSLRPEDTALYYCNKVPWGDYHPGNVYWGQGTQVTVSS

SEQ ID NO: 135 - protein, 119 aa, synthetic construct
EVQLVESGGGLVQPGRSLTLSCAASTNIVSISPMGWYRQAPGKQRELVAAIHGHSTLYADSVKGRFTISRDNAKNSIYLQMNSLRPEDTALYYCNKVPWGDYHPGNVYWGQGTQVTVSS

SEQ ID NO: 136 - protein, 119 aa, synthetic construct
EVQLVESGGGLVQPGRSLTLSCAASTNIVSISPMGWYRQAPGKQRELVAAIHGKSTLYADSVKGRFTISRDNAKNSIYLQMNSLRPEDTALYYCNKVPWGDYHPGNVYWGQGTQVTVSS

SEQ ID NO: 137 - protein, 119 aa, synthetic construct
EVQLVESGGGLVQPGRSLTLSCAASTNVVSISPMGWYRQAPGKQRELVAAIHGKSTLYADSVKGRFTISRDNAKNSIYLQMNSLRPEDTALYYCNKVPWGDYHPNNVYWGQGTQVTVSS

SEQ ID NO: 138 - protein, 119 aa, synthetic construct
EVQLVESGGGLVQPGRSLTLSCAASTNIISISPMGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 139 - protein, 119 aa, synthetic construct
EVQLVESGGGLVQPGRSLTLSCAASTNIFSITPMGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 140 - protein, 119 aa, synthetic construct
EVQLVESGGGLVQPGRSLTLSCAASTNITSISPMGWYRQAPGKQRELVAAIHGFETLYADSVKGRFTISRDNAKNSIYLQMNSLRPEDTALYYCNKVPWGDYHPGNVYWGQGTQVTVSS

SEQ ID NO: 141 - protein, 119 aa, synthetic construct
EVQLVESGGGLVQPGRSLTLSCAASTNIQSISPMGWYRQAPGKQRELVAAIHGFETLYADSVKGRFTISRDNAKNSIYLQMNSLRPEDTALYYCNKVPWGDYHPGNVYWGQGTQVTVSS

SEQ ID NO: 142 - protein, 119 aa, synthetic construct
EVQLVESGGGLVQPGRSLTLSCAASTSDFSISPMGWYRQAPGKQRELVAAIHGFETLYADSVKGRFTISRDNAKNSIYLQMNSLRPEDTALYYCNKVPWGDYHPGNVYWGQGTQVTVSS

SEQ ID NO: 143 - protein, 119 aa, synthetic construct
EVQLVESGGGLVQPGRSLTLSCAASTNIDSISPMGWYRQAPGKQRELVAAIHGFQTLYADSVKGRFTISRDNAKNSIYLQMNSLRPEDTALYYCNKVPWGDYHPGNVYWGQGTQVTVSS

SEQ ID NO: 144 - protein, 119 aa, synthetic construct
EVQLVESGGGLVQPGRSLTLSCAASTNIMSISPMGWYRQAPGKQRELVAAIHGFSTVYADSVKGRFTISRDNAKNSIYLQMNSLRPEDTALYYCNKVPWGDYHPGNVYWGQGTQVTVSS

SEQ ID NO: 145 - protein, 119 aa, synthetic construct
EVQLVESGGGLVQPGRSLTLSCAASTNIESISPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 146 - protein, 119 aa, synthetic construct
EVQLVESGGGLVQPGRSLTLSCAASTNIFSSSPMGWYRQAPGKQRELVAAIHGFKTLYADSVKGRFTISRDNAKNSIYLQMNSLRPEDTARYYCNKVPWGDYHPGNVYWGQGTQVTVSS

SEQ ID NO: 147 - protein, 119 aa, synthetic construct
EVQLVESGGGLVQPGRSLTLSCAASTNIFSSSPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 148 - protein, 119 aa, synthetic construct
EVQLVESGGGLVQPGRSLTLSCAASTNIFSNSPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 149 - protein, 119 aa, synthetic construct
EVQLVESGGGLVQPGRSLTLSCAASTNIFSTSPMGWYRQAPGKQRELVAAIHGFSTIYADSVKGRFTISRDNAKNSIYLQMNSLRPEDTALYYCNKVPWGDYHPGNVYWGQGTQVTVSS

SEQ ID NO: 150 - protein, 119 aa, synthetic construct
EVQLVESGGGLVQPGRSLTLSCAASTNIFSTSPMGWYRQAPGKQRELVAAIHGFSTIYADSVKGRFTISRDNAKNSIYLQMNSLRPEDTALYYCNKVPWGDYHPLNVYWGQGTQVTVSS

SEQ ID NO: 151 - protein, 119 aa, synthetic construct
EVQLVESGGGLVQPGRSLTLSCVASTNIFSTSPMGWYRQAPGKQRELVAAIHGFSTLYADSVKGRFTISRDNAKNSIYLQMNSLRPEDTALYYCNKVPWGDYHPGNVYWGQGTQVTVSS

SEQ ID NO: 152 - protein, 119 aa, synthetic construct
EVQLVESGGGLVQPGRSLTLSCAASTNIFSDSPMGWYRQAPGKQRELVAAIHGFSTFYADSVKGRFTISRDNAKNSIYLQMNSLRPEDTALYYCNKVPWGDYHPGNVYWGQGTQVTVSS

SEQ ID NO: 153 - protein, 119 aa, synthetic construct
EVQLVESGGGLVQPGRSLTLSCAASTNIFSQSPMGWYRQAPGKQRELVAAIHGDSTLYADSVKGRFTISRDNAKNSIYLQMNSLRPEDTALYYCNKVPWGDYHPGNVCWGQGTQVTVSS

SEQ ID NO: 154 - protein, 119 aa, synthetic construct
EVQLVESGGGLVQPGRSLTLSCAASTNIFSQSPMGWYRQAPGKQRELVAAIHGKSTLYADSVKGRFTISRDNAKNSIYLQMNSLRPEDTALYYCNKVPWGDYHPSNVYWGKGTQVTVSS

SEQ ID NO: 155 - protein, 119 aa, synthetic construct
EVQLVESGGGLVQPGRSLTLSCAASTNIFSRSPMGWYRQAPGKQRELVAAIHGESTLYADSVKGRFTISRDNAKNSIYLQMNSLRPEDTALYYCNKVPWGRYHPGNVYWGQGTQVTVSS

SEQ ID NO: 156 - protein, 119 aa, synthetic construct
EVQLVESGGGLVQPGRSLTLSCAASTNIFSRSPMGWYRQAPGKQRELVAAIHGISTLYADSVKGRFTISRDNAKNSIYLQMNSLRPEDTALYYCNKVPWGDYHPGNVYWGQGTQVTVSS

SEQ ID NO: 157 - protein, 119 aa, synthetic construct
EVQLVESGGGLVQPGRSLTLSCAASTNIFSRSPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 158 - protein, 119 aa, synthetic construct
EVQLVESGGGLVQPGRSLTLSCAASTNIFSGSPMGWYRQAPGKQRELVAAIHGNSTLYADSVKGRFTISRDNAKNSIYLQMNSLRPEDTALYYCNKVPWGDYHPGNVYWGQGTQVTVSS

SEQ ID NO: 159 - protein, 119 aa, synthetic construct
EVQLVESGGGLVQPGRSLTLSCAASSNIFSISPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 160 - protein, 119 aa, synthetic construct
EVQLVESGGGLVQPGRSLTLSCAASTNIFSIYPMGWYRQAPGKQRELVAAIHGSSTLYADSVKGRFTISRDNAKNSIYLQMNSLRPEDTALYYCNKVPWGDYHPKNVYWGQGTQVTVSS

SEQ ID NO: 161 - protein, 119 aa, synthetic construct
EVQLVESGGGLVQPGRSLTLSCAASTNIFSKSPMGWYRQAPGKQRELVAAIHGSSTLYADSVKGRFTISRDNAKNSIYLQMNSLRPEDTALYYCNKVPWGDYHPGNVYWGQGTQVTVSS

SEQ ID NO: 162 - protein, 119 aa, synthetic construct
EVQLVESGGGLVQPGRSLTLSCAASTNIFSKSPMGWYRQAPGKQRELVAAIHGSSTLYADSVKGRFTISRDNAKNSIYLQMNSLRPEDTALYYCNKVPWGDYHPRNVYWGQGTQVTVSS

SEQ ID NO: 163 - protein, 119 aa, synthetic construct
EVQLVESGGGLVQPGRSLTLSCAASTNEFSISPMGWYRQAPGKQRELVAAIHGLSTLYADSVKGRFTISRDNAKNSIYLQMNSLRPEDTALYYCNKVPWGAYHPRNVYWGQGTQVTVSS

SEQ ID NO: 164 - protein, 119 aa, synthetic construct
EVQLVESGGGLVQPGRSLTLSCAASTNEFSISPMGWYRQAPGKQRELVAAIHGESTLYADSVKGRFTISRDNAKNSIYLQMNSLRPEDTALYYCNKVPWGDYHPGNVYWGQGTQVTVSS

SEQ ID NO: 165 - protein, 119 aa, synthetic construct
EVQLVESGGGLVQPGRSLTLSCAASTNIPSISPMGWYRQAPGKQRELVAAIHGESTLYADSVKGRFTISRDNAKNSIYLQMNSLRPEDTALYYCNKVPWGDYHPRNVAWGQGTQVTVSS

SEQ ID NO: 166 - protein, 119 aa, synthetic construct
EVQLVESGGGLVQPGRSLTLSCAASTNIPSISPMGWYRQAPGKQRELVAAIHGASTLYADSVKGRFTISRDNAKNSIYLQMNSLRPEDTALYYCNKVPWGDYHPRNVAWGQGTQVTVSS

SEQ ID NO: 167 - protein, 119 aa, synthetic construct
EVQLVESGGGLVQPGRSLTLSCAASTNIPSISPMGWYRQAPGKQRELVAAIHGESTLYADSVKGRFTISRDNAKNSIYLQMNSLRPEDTALYYCNKVPWGDYHPRNVYWGQGTQVTVSS

SEQ ID NO: 168 - protein, 119 aa, synthetic construct
EVQLVESGGGLVQPGRSLTLSCAASTNIPSISPMGWYRQAPGKQRELVAAIHGDSTLYADSVKGRFTISRDNAKNSIYLQMNSLRPEDTALYYCNKVPWGDYHPRNVYWGQGTQVTVSS

SEQ ID NO: 169 - protein, 119 aa, synthetic construct
EVQLVESGGGLVQPGRSLTLSCAASTNITSISPMGWYRQAPGKQRELVAAIHGVSTLYADSVKGRFTISRDNAKNSIYLQMNSLRPEDTALYYCNKVPWGDYHPRNVQWGQGTQVTVSS

SEQ ID NO: 170 - protein, 119 aa, synthetic construct
EVQLVESGGGLVQPGRSLTLSCAASTNIPSISPMGWYRQAPGKQRELVAAIHGQSTLYADSVKGRFTISRDNAKNSIYLQMNSLRPEDTALYYCNKVPWGDYHPRNVQWGQGTQVTVSS

SEQ ID NO: 171 - protein, 119 aa, synthetic construct
EVQLVESGGGLVQPGRSLTLSCAASTNIVSISPMGWYRQAPGKQRELVAAIHGDSTLYADSVKGRFTISRDNAKNSIYLQMNSLRPEDTALYYCNKVPWGDYHPRNVSWGQGTQVTVSS

SEQ ID NO: 172 - protein, 119 aa, synthetic construct
EVQLVESGGGLVQPGRSLTLSCAASSNIFSISPMGWYRQAPGKQRELVAAIHGESTLYADSVKGRFTISRDNAKNSIYLQMNSLRPEDTALYYCNKVPWGDYHPRNVTWGQGTQVTVSS

SEQ ID NO: 173 - protein, 119 aa, synthetic construct
EVQLVESGGGLVQPGRSLTLSCAASTNIDSISPMGWYRQAPGKQRELVAAIHGESTLYADSVKGRFTISRDNAKNSIYLQMNSLRPEDTALYYCNKVPWGDYHPRNVTWGQGTQVTVSS

SEQ ID NO: 174 - protein, 119 aa, synthetic construct
EVQLVESGGGLVQPGRSLTLSCAASTNIDSISPMGWYRQAPGKQRELVAAIHGSSTLYADSVKGRFTISRDNAKNSIYLQMNSLRPEDTALYYCNKVPWGDYHPRNVTWGQGTQVTVSS

SEQ ID NO: 175 - protein, 119 aa, synthetic construct
EVQLVESGGGLVQPGRSLTLSCAASTNIRSISPMGWYRQAPGKQRELVAAIHGSSTLYADSVKGRFTISRDNAKNSIYLQMNSLRPEDTALYYCNKVPWGDYHPRNVVWGQGTQVTVSS

SEQ ID NO: 176 - protein, 119 aa, synthetic construct
EVQLVESGGGLVQPGRSLTLSCAASTNITSISPMGWYRQAPGKQRELVAAISGFSTLYADSVKGRFTISRDNAKNSIYLQMNSLRPEDTALYYCNEVPWGDYHPRNVYWGQGTQVTVSS

SEQ ID NO: 177 - protein, 119 aa, synthetic construct
EVQLVESGGGLVQPGRSLTLSCAASTNITSISPMGWYRQAPGKQRELVAAIHGESTLYADSVKGRFTISRDNAKNSIYLQMNSLRPEDTALYYCNKVPWGDYHPRNVYWGQGTQVTVSS

SEQ ID NO: 178 - protein, 119 aa, synthetic construct
EVQLVESGGGLVQPGRSLTLSCAASTNITSVSPMGWYRQAPGKQRELVAAIHGPSTLYADSVKGRFTISRDNAKNSIYLQMNSLRPEDTALYYCNKVPWGDYHPTNVYWGQGTQVTVSS

SEQ ID NO: 179 - protein, 119 aa, synthetic construct
EVQLVESGGGLVQPGRSLTLSCAASTNIGSISPMGWYRQAPGKQRELVAAIHGQSTLYADSVKGRFTISRDNAKNSIYLQMNSLRPEDTALYYCNKVPWGDYHPQNVYWGQGTQVTVSS

SEQ ID NO: 180 - protein, 119 aa, synthetic construct
EVQLVESGGGLVQPGRSLTLSCAASTNIESISPMGWYRQAPGKQRELVAAIHGKSTLYADSVKGRFTISRDNAKNSIYLQMNSLRPEDTALYYCNKVPWGDYHPRRVYWGQGTQVTVSS

SEQ ID NO: 181 - protein, 119 aa, synthetic construct
EVQLVESGGGLVQPGGSLTLSCVASGSTSSINAMGWYRRAPGKQRELVAGVSSDGSKVYADSVKGRFTISRDNAKNSVYLQMNSLRAEDTAVYYCYYFRTVSGSSARYWGQGTLVTVSS

SEQ ID NO: 182 - protein, 119 aa, synthetic construct
EVQLVESGGGLVQPGGSLTLSCVASGSTSSINAMGWYRRAPGKQRELPAGISRDGSKVYADSVKGRFTISRDNAKNSVYLQMNSLRAEDTAVYYCYYFRYVSGSSMRYWGQGTLVTVSS

SEQ ID NO: 183 - protein, 119 aa, synthetic construct
EVQLVESGGGLVQPGGSLTLSCVASGSTSSINAMGWYRRAPGKQRELAAGISSDGSKLYADSVKGRFTISRDNAKNSVYLQMNSLRAEDTAVYYCYYFRTVSGSSMRYWGQGTLVTVSS

SEQ ID NO: 184 - protein, 119 aa, synthetic construct
EVQLVESGGGLVQPGGSLTLSCVASGSTSRINAMGWYRRAPGKQRELVAGISSDGSKVYADSVKGRFTISRDNAKNSVYLQMNSLRAEDTAVYYCYYFRRVSGSSMRYWGQGTLVTVSS

SEQ ID NO: 185 - protein, 119 aa, synthetic construct
EVQLVESGGGLVQPGGSLTLSCVASGSTSSINAMAWYRRAPGKQRELAAGISSDGSKVYADSVKGRFTISRDNAKNSVYLQMNSLRAEDTAVYYCYYFHTVSGSSMRYWGQGTLVTVSS

SEQ ID NO: 186 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRQVSGSSMRYWGQGTLVTVSS

SEQ ID NO: 187 - protein, 119 aa, synthetic construct
EVQLVESGGGLVQPGGSLTLSCVASGSTSSINAMGWYRRAPGKQRELVAGISSDTSKVYADSVKGRFTISRDNAKNSVYLQMNSLRAEDTAVYYCYYFRTVSGSYMRYWGQGTLVTVSS

SEQ ID NO: 188 - protein, 119 aa, synthetic construct
EVQLVESGGGLVQPGGSLTLSCVASGSTSTINAMGWYRRAPGKQRELVAGISSDGSKVYADSVKGRFTISRDNAKNSVYLQMNSLRAEDTAVYYCYYFRTASGSSMRYWGQGTLVTVSS

SEQ ID NO: 189 - protein, 119 aa, synthetic construct
EVQLVESGGGLVQPGRSLTLSCAASTNIVSISPMGWYRQAPGKQRELVAAIHGDSTLYADSVKGRFTISRDNAKNSIYLQMNSLRPEDTALYYCNKVPWGDYHPRRVYWGQGTQVTVSS

SEQ ID NO: 190 - protein, 119 aa, synthetic construct
EVQLVESGGGLVQPGRSLTLSCAASTNIDSISPMGWYRQAPGKQRELVAAIHGNSTLYADSVKGRFTISRDNAKNSIYLQMNSLRPEDTALYYCNKVPWGDYHPRMVYWGQGTQVTVSS

SEQ ID NO: 191 - protein, 119 aa, synthetic construct
EVQLVESGGGLVQPGRSLTLSCAASTNIFMISPMGWYRQAPGKQRELVAAIHGDSTLYADSVKGRFTISRDNAKNSIYLQMNSLRPEDTALYYCNKVPWGRYHPRNVYWGQGTQVTVSS

SEQ ID NO: 192 - protein, 119 aa, synthetic construct
EVQLVESGGGLVQPGRSLTLSCAASTNIFRISPMGWYRQAPGKQRELVAAIHGDSTLYADSVKGRFTISRDNAKNSIYLQMNSLRPEDTALYYCNKVPWGRYHPRNVYWGQGTQVTVSS

SEQ ID NO: 193 - protein, 119 aa, synthetic construct
EVQLVESGGGLVQPGRSLTLSCAASTNIFSISPMGWYRQAPGKQRELVAAIHGDSTLYADSVKGRFTISRDNAKNSIYLQMNSLRPEDTALYYCNKVPWGEYHPRNVYWGQGTQVTVSS

SEQ ID NO: 194 - protein, 119 aa, synthetic construct
EVQLVESGGGLVQPGRSLTLSCAASTNIFSISPMGWYRQAPGKQRELVAAIHGDSTLYADSVKGRFTISRDNAKNSIYLQMNSLRPEDTALYYCNKVPWGKYHPRNVYWGQGTQVTVSS

SEQ ID NO: 195 - protein, 119 aa, synthetic construct
EVQLVESGGGLVQPGRSLTLSCAASTNIESISPMGWYRQAPGKQRELVAAIHGSSTLYADSVKGRFTISRDNAKNSIYLQMNSLRPEDTALYYCNKVPWGRYHPRNVYWGQGTQVTVSS

SEQ ID NO: 196 - protein, 119 aa, synthetic construct
EVQLVESGGGLVQPGRSLTLSCAASTNIESISPMGWYRQAPGKQRELVAAIHGNSTLYADSVKGRFTISRDNAKNSIYLQMNSLRPEDTALYYCNKVPWGRYHPRNVYWGQGTQVTVSS

SEQ ID NO: 197 - protein, 119 aa, synthetic construct
EVQLVESGGGLVQPGRSLTLSCAASTNISSISPMGWYRQAPGKQRELVAAIHGFSTLYADSVKGRFTISRDNAKNSIYLQMNSLRPEDTALYYCNKVPWGYYHPRNVYWGQGTQVTVSS

SEQ ID NO: 198 - protein, 119 aa, synthetic construct
EVQLVESGGGLVQPGRSLTLSCAASTNISSISPMGWYRQAPGKQRELVAAIHGHSTLYADSVKGRFTISRDNAKNSIYLQMNSLRPEDTALYYCNKVPWGRYHPRNVYWGQGTQVTVSS

SEQ ID NO: 199 - protein, 119 aa, synthetic construct
EVQLVESGGGLVQPGRSLTLSCAASTNISSISPMGWYRQAPGKQRELVAAIHGFSTVYADSVKGRFTISRDNAKNSIYLQMNSLRPEDTALYYCNKVPWGRYHPRNVYWGQGTQVTVSS

SEQ ID NO: 200 - protein, 119 aa, synthetic construct
EVQLVESGGGLVQPGRSLTLSCAASTNIFSIRPMGWYRQAPGKQRELVAAIHGFSTVYADSVKGRFTISRDNAKNSIYLQMNSLRPEDTALYYCNKVPWGDYHPRNVYWGQGTQVTVSS

SEQ ID NO: 201 - protein, 119 aa, synthetic construct
EVQLVESGGGLVQPGRSLTLSCAASTNIFSIYPMGWYRQAPGKQRELVAAIHGFSTYYADSVKGRFTISRDNAKNSIYLQMNSLRPEDTALYYCNKVPWGSYHPRNVYWGQGTQVTVSS

SEQ ID NO: 202 - protein, 119 aa, synthetic construct
EVQLVESGGGLVQPGRSLTLSCAASTNIFNISPMGWYRQAPGKQRELVAAIHGFSTYYADSVKGRFTISRDNAKNSIYLQMNSLRPEDTALYYCNKVPWGRYHPRNVYWGQGTQVTVSS

SEQ ID NO: 203 - protein, 119 aa, synthetic construct
EVQLVESGGGLVQPGRSLTLSCAASTNIFSSSPMGWYRQAPGKQRELVAAIHGFSTWYADSVKGRFTISRDNAKNSIYLQMNSLRPEDTALYYCNKVPWGRYHPRNVYWGQGTQVTVSS

SEQ ID NO: 204 - protein, 119 aa, synthetic construct
EVQLVESGGGLVQPGRSLTLSCAASTNISSISPMGWYRQAPGKQRELVAAIHGFDTLYADSVKGRFTISRDNAKNSIYLQMNSLRPEDTALYYCNKVPWGDYHPRNVYWGQGTQVTVSS

SEQ ID NO: 205 - protein, 119 aa, synthetic construct
EVQLVESGGGLVQPGRSLTLSCAASTNIFSINPMGWYRQAPGKQRELVAAIHGFDTLYADSVKGRFTISRDNAKNSIYLQMNSLRPEDTALYYCNKVPWGDYHPRNVSWGQGTQVTVSS

SEQ ID NO: 206 - protein, 119 aa, synthetic construct
EVQLVESGGGLVQPGRSLTLSCAASTNIFSITPMGWYRQAPGKQRELVAAIHGRSTLYADSVKGRFTISRDNAKNSIYLQMNSLRPEDTALYYCNKVPWGSYHPRNVYWGQGTQVTVSS

SEQ ID NO: 207 - protein, 119 aa, synthetic construct
EVQLVESGGGLVQPGRSLTLSCAASTNIFSITPMGWYRQAPGKQRELVAAIHGTSTLYADSVKGRFTISRDNAKNSIYLQMNSLRPEDTALYYCNKVPWGRYHPRNVYWGQGTQVTVSS

SEQ ID NO: 208 - protein, 119 aa, synthetic construct
EVQLVESGGGLVQPGRSLTLSCAASTNIFSITPMGWYRQAPGKQRELVAAIHGESTLYADSVKGRFTISRDNAKNSIYLQMNSLRPEDTALYYCNKVPWGRYHPRNVYWGQGTQVTVSS

SEQ ID NO: 209 - protein, 119 aa, synthetic construct
EVQLVESGGGLVQPGRSLTLSCAASTNIFSITPMGWYRQAPGKQRELVAAIHGESTLYADSVKGRFTISRDNAKNSIYLQMNSLRPEDTALYYCNKVPWGDYHPRDVYWGQGTQVTVSS

SEQ ID NO: 210 - protein, 119 aa, synthetic construct
EVQLVESGGGLVQPGRSLTLSCAASTNIFSTSPYGWYRQAPGKQRELVAAIHGFSTIYADSVKGRFTISRDNAKNSIYLQMNSLRPEDTALYYCNKVPWGDYHPGNVYWGQGTQVTVSS

SEQ ID NO: 211 - protein, 119 aa, synthetic construct
EVQLVESGGGLVQPGRSLTLSCAASTNIFSTSPGGWYRQAPGKQRELVAAIHGFSTIYADSVKGRFTISRDNAKNSIYLQMNSLRPEDTALYYCNKVPWGDYHPGNVYWGQGTQVTVSS

SEQ ID NO: 212 - protein, 119 aa, synthetic construct
EVQLVESGGGLVQPGRSLTLSCAASTNIFSITPYGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 213 - protein, 119 aa, synthetic construct
EVQLVESGGGLVQPGRSLTLSCAASTNIFSITPGGWYRQAPGKQRELVAAIHGASTLYADSVKGRFTISRDNAKNSIYLQMNSLRPEDTALYYCNKVPWGDYHPGNVYWGQGTQVTVSS

SEQ ID NO: 214 - protein, 119 aa, synthetic construct
QVQLVESGGGLVQPGESLRLSCAASTNIFSISPMGWYRQAPGKQRELVAAIHGFSTLYADSVKGRFTISRDNAKNTIYLQMNSLKPEDTAVYYCNKVPWGDYHPRNVYWGQGTQVTVSS

SEQ ID NO: 215 - protein, 110 aa, synthetic construct
QVQLVESGGGLVQPGGSLRLSCAASGRTFSVRGMAWYRQAGNNRALVATMNPDGFPNYADAVKGRFTISWDIAENTVYLQMNSLNSEDTTVYYCNSGPYWGQGTQVTVSS

SEQ ID NO: 216 - protein, 123 aa, synthetic construct
QVQLVESGGGLVQAGGSLRLSCAASGSIPSIEQMGWYRQAPGKQRELVAALTSGGRANYADSVKGRFTISGDNVRNMVYLQMNSLKPEDTAIYYCSAGRFKGDYAQRSGMDYWGKGTLVTVSS

SEQ ID NO: 217 - protein, 116 aa, synthetic construct
QVQLVESGGGLVQAGGSLRLSCAFSGTTYTFDLMSWYRQAPGKQRTVVASISSDGRTSYADSVRGRFTISGENGKNTVYLQMNSLKLEDTAVYYCLGQRSGVRAFWGQGTQVTVSS

SEQ ID NO: 218 - protein, 123 aa, synthetic construct
QVQLVESGGGLVQAGGSLRLSCVASGSTSNINNMRWYRQAPGKERELVAVITRGGYAIYLDAVKGRFTISRDNANNAIYLEMNSLKPEDTAVYVCNADRVEGTSGGPQLRDYFGQGTQVTVSS

SEQ ID NO: 219 - protein, 116 aa, synthetic construct
QVQLVESGGGLVQAGGSLRLSCAASGSTFGINAMGWYRQAPGKQRELVAVISRGGSTNYADSVKGRFTISRDNAENTVSLQMNTLKPEDTAVYFCNARTYTRHDYWGQGTQVTVSS

SEQ ID NO: 220 - protein, 122 aa, synthetic construct
QVRLVESGGGLVQAGGSLRLSCAASISAFRLMSVRWYRQDPSKQREWVATIDQLGRTNYADSVKGRFAISKDSTRNTVYLQMNMLRPEDTAVYYCNAGGGPLGSRWLRGRHWGQGTQVTVSS

SEQ ID NO: 221 - protein, 119 aa, synthetic construct
QVRLVESGGGLVQAGESLRLSCAASGRPFSINTMGWYRQAPGKQRELVASISSSGDFTYTDSVKGRFTISRDNAKNTVYLQMNSLKPEDTAVYYCNARRTYLPRRFGSWGQGTQVTVSS

SEQ ID NO: 222 - protein, 117 aa, synthetic construct
QVQPVESGGGLVQPGGSLRLSCVVSGSDFTEDAMAWYRQASGKERESVAFVSKDGKRILYLDSVRGRFTISRDIDKKTVYLQMDNLKPEDTGVYYCNSAPGAARNYWGQGTQVTVSS

SEQ ID NO: 223 - protein, 117 aa, synthetic construct
QVQPVESGGGLVQPGGSLRLSCVVSGSDFTEDAMAWYRQASGKERESVAFVSKDGKRILYLDSVRGRFTISRDIYKKTVYLQMDNLKPEDTGVYYCNSAPGAARNVWGQGTQVTVSS

SEQ ID NO: 224 - protein, 115 aa, synthetic construct [sequence omitted]

SEQ ID NO: 225 - protein, 124 aa, synthetic construct
QVQIVESGGGLVQAGGSLRLSCVASGLTYSIVAVGWYRQAPGKEREMVADISPVGNTNYADSVKGRFTISKENAKNTVYLQMNSLKPEDTAVYYCHIVRGWLDERPGPGPIVYWGQGTQVTVSS

SEQ ID NO: 226 - protein, 115 aa, synthetic construct
QVQLVESGGGLVQTGGSLRLSCAASGLTFGVYGMEWFRQAPGKQREWVASHTSTGYVYYRDSVKGRFTISRDNAKSTVYLQMNSLKPEDTAIYYCKANRGSYEYWGQGTQVTVSS

SEQ ID NO: 227 - protein, 114 aa, synthetic construct
QVQLVESGGGLVQAGGSLRLSCAASTTSSINSMSWYRQAQGKQREPVAVITDRGSTSYADSVKGRFTISRDNAKNTVYLQMNSLKPEDTAIYTCHVIADWRGYWGQGTQVTVSS

SEQ ID NO: 228 - protein, 120 aa, synthetic construct
QVQLVESGGGLVQAGGSLRLSCAASGRTLSRYAMGWFRQAPGKERQFVAAISRSGGTTRYSDSVKGRFTISRDNAANTFYLQMNNLRPDDTAVYYCNVRRRGWGRTLEYWGQGTQVTVSS

SEQ ID NO: 229 - protein, 117 aa, synthetic construct
QVQLGESGGGLVQAGGSLRLSCAASGSIFSPNAMIWHRQAPGKQREPVASINSSGSTNYGDSVKGRFTVSRDIVKNTMYLQMNSLKPEDTAVYYCSYSDFRRGTQYWGQGTQVTVSS

SEQ ID NO: 230 - protein, 119 aa, synthetic construct
QVQLVESGGGLVPSGGSLRLSCAASGATSAITNLGWYRRAPGQVREMVARISVREDKEDYEDSVKGRFTISRDNTQNLVYLQMNNLQPHDTAIYYCGAQRWGRGPGTTWGQGTQVTVSS

SEQ ID NO: 231 - protein, 116 aa, synthetic construct
QVQLVESGGGLVQAGGSLRLSCAASGSTFRIRVMRWYRQAPGTERDLVAVISGSSTYYADSVKGRFTISRDNAKNTLYLQMNNLKPEDTAVYYCNADDSGIARDYWGQGTQVTVSS

SEQ ID NO: 232 - protein, 118 aa, synthetic construct
QVQLVESGGGLVQAGESRRLSCAVSGDTSKFKAVGWYRQAPGAQRELLAWINNSGVGNTAESVKGRFTISRDNAKNTVYLQMNRLTPEDTDVYYCRFYRRFGINKNYWGQGTQVTVSS

SEQ ID NO: 233 - protein, 122 aa, synthetic construct
QVQLVESGGGLVQAGGSLRLSCAASGSTFGNKPMGWYRQAPGKQRELVAVISSDGGSTRYAALVKGRFTISRDNAKNTVYLQMESLVAEDTAVYYCNALRTYYLNDPVVFSWGQGTQVTVSS

SEQ ID NO: 234 - protein, 119 aa, synthetic construct
QVQLVESGGGLVQAGGSLRLSCAASGSTSSINTMYWYRQAPGKERELVAFISSGGSTNVRDSVKGRFSVSRDSAKNIVYLQMNSLTPEDTAVYYCNTYIPLRGTLHDYWGQGTQVTVSS

SEQ ID NO: 235 - protein, 115 aa, synthetic construct
QVQLVESGGGLVQAGGSLRLSCVASGRTDRITTMGWYRQAPGKQRELVATISNRGTSNYANSVKGRFTISRDNAKNTVYLQMNSLKPEDTAVYYCNARKWGRNYWGQGTQVTVSS

SEQ ID NO: 236 - protein, 119 aa, synthetic construct
QVQLVESGGGLVQARGSLRLSCTASGRTIGINDMAWYRQAPGNQRELVATITKGGTTDYADSVDGRFTISRDNAKNTVYLQMNSLKPEDTAVYYCNTKRREWAKDFEYWGQGTQVTVSS

SEQ ID NO: 237 - protein, 114 aa, synthetic construct
QVQLVESGGGLVQAGGSLRLSCAASAIGSINSMSWYRQAPGKQREPVAVITDRGSTSYADSVKGRFTISRDNAKNTVYLQMNSLKPEDTAIYTCHVIADWRGYWGQGTQVTVSS

SEQ ID NO: 238 - protein, 119 aa, synthetic construct
QVQLVESGGGLVQAGGSLRLSCAASGSTSSINTMYWFRQAPGEERELVATINRGGSTNVRDSVKGRFSVSRDSAKNIVYLQMNRLKPEDTAVYYCNTYIPYGGTLHDFWGQGTQVTVSS

SEQ ID NO: 239 - protein, 114 aa, synthetic construct
QVQLVESGGGLVQAGGSLRLSCTTSTTFSINSMSWYRQAPGNQREPVAVITNRGTTSYADSVKGRFTISRDNARNTVYLQMDSLKPEDTAIYTCHVIADWRGYWGQGTQVTVSS

SEQ ID NO: 240 - protein, 116 aa, synthetic construct
QVQLVESGGGLVQAGGSLTLSCAASGSTFSIRAMRWYRQAPGTERDLVAVIYGSSTYYADAVKGRFTISRDNAKNTLYLQMNNLKPEDTAVYYCNADTIGTARDYWGQGTQVTVSS

SEQ ID NO: 241 - protein, 117 aa, synthetic construct
QVQLVESGGGLVQAGGSLRLSCVASGRTSTIDTMYWHRQAPGNERELVAYVTSRGTSNVADSVKGRFTISRDNAKNTAYLQMNSLKPEDTAVYYCSVRTTSYPVDFWGQGTQVTVSS

SEQ ID NO: 242 - protein, 119 aa, synthetic construct
QVQLVESGGGLVQAGGSLRLSCAASGSTSSINTMYWYRQAPGKERELVAFISSGGSTNVRDSVKGRFSVSRDSAKNIVYLQMNSLKPEDTAVYYCNTYIPYGGTLHDFWGQGTQVTVSS

SEQ ID NO: 243 - protein, 111 aa, synthetic construct
QVQLVESGGGLVQPGGSLRLSCAASGGDWSANFMYWYRQAPGKQRELVARISGRGVVDYVESVKGRFTISRDNAKNTVYLQMNSLKPEDTAVYYCAVASYWGQGTQVTVSS

SEQ ID NO: 244 - protein, 111 aa, synthetic construct
EVQLVESGGGLVQPGGSLRLSCAASGGDWSANFMYWYRQAPGKQRELVARISGRGVVDYVESVKGRFTISRDNSKNTLYLQMNSLRAEDTAVYYCAVASYWGQGTLVTVSS

SEQ ID NO: 245 - protein, 111 aa, synthetic construct
EVQLVESGGGLVQPGGSLRLSCAASGGDWSANFMYWVRQAPGKGLEWVSRISGRGVVDYVESVKGRFTISRDNSKNTLYLQMNSLRAEDTAVYYCAVASYWGQGTLVTVSS

SEQ ID NO: 246 - protein, 119 aa, synthetic construct
EVQLVESGGGLVQAGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 247 - protein, 119 aa, synthetic construct
EVQLVESGGGLVQPGGSLRLSCAASGSTSSINTMYWYRQAPGKERELVAFISSGGSTNVRDSVKGRFTISRDNSKNTLYLQMNSLRAEDTAVYYCNTYIPYGGTLHDFWGQGTLVTVSS

SEQ ID NO: 248 - protein, 119 aa, synthetic construct
EVQLVESGGGLVQPGGSLRLSCAASGSTSSINTMYWVRQAPGKGLEWVSFISSGGSTNVRDSVKGRFTISRDNSKNTLYLQMNSLRAEDTAVYYCNTYIPYGGTLHDFWGQGTLVTVSS

SEQ ID NO: 249 - protein, 116 aa, synthetic construct
QVQLVESGGGVVQAGGSLRLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 250 - protein, 118 aa, synthetic construct
QVQLVESGGGVVQPGGSLRLSCAASGSTFSIRAMRWYRQAPGKERELVAVIYGSSTYYADAVKGRFTISRDNSKNTLYLQMNSLRAEDTAVYYCNADTIGTARDYWGQGTLVTVSSGG

SEQ ID NO: 251 - protein, 118 aa, synthetic construct
QVQLVESGGGVVQPGGSLRLSCAASGSTFSIRAMRWVRQAPGKGLEWVSVIYGSSTYYADAVKGRFTISRDNSKNTLYLQMNSLRAEDTAVYYCNADTIGTARDYWGQGTLVTVSSGG

SEQ ID NO: 252 - protein, 118 aa, synthetic construct
EVQLVESGGGLVQAGGSLRLSCVASGRTSTIDTMYWHRQAPGNERELVAYVTSRGTSNVADSVKGRFTISRDNSKNTLYLQMNSLRAEDTAVYYCSVRTTSYPVDFWGQGTLVTVSGG

SEQ ID NO: 253 - protein, 117 aa, synthetic construct
EVQLVESGGGLVQPGGSLRLSCAASGRTSTIDTMYWHRQAPGKERELVAYVTSRGTSNVADSVKGRFTISRDNSKNTLYLQMNSLRAEDTAVYYCSVRTTSYPVDFWGQGTLVTVSS

SEQ ID NO: 254 - protein, 117 aa, synthetic construct
EVQLVESGGGLVQPGGSLRLSCAASGRTSTIDTMYWVRQAPGKGLEWVSYVTSRGTSNVADSVKGRFTISRDNSKNTLYLQMNSLRAEDTAVYYCSVRTTSYPVDFWGQGTLVTVSS

SEQ ID NO: 255 - protein, 116 aa, synthetic construct
QVQLVESGGGVVQAGGSLTLSCAASGSTFSIRAMRWYRQAPGTERDLVAVIYGSSTYYADAVKGRFTISRDNSKNTLYLQMNSLRAEDTAVYYCNADTIGTARDYWGQGTLVTVSS

SEQ ID NO: 256 - protein, 116 aa, synthetic construct; identical to SEQ ID NO: 249

SEQ ID NO: 257 - protein, 116 aa, synthetic construct
QVQLVESGGGVVQPGGSLRLSCAASGSTFSIRAMRWYRQAPGKERELVAVIYGSSTYYADAVKGRFTISRDNSKNTLYLQMNSLRAEDTAVYYCNADTIGTARDYWGQGTLVTVSS

SEQ ID NO: 258 - protein, 116 aa, synthetic construct
QVQLVESGGGVVQPGGSLRLSCAASGSTFSIRAMRWVRQAPGKGLEWVSVIYGSSTYYADAVKGRFTISRDNSKNTLYLQMNSLRAEDTAVYYCNADTIGTARDYWGQGTLVTVSS

SEQ ID NO: 259 - protein, 12 aa, synthetic construct
GGQDGNEEMGGG

SEQ ID NO: 260 - protein, 10 aa, synthetic construct
GGQDGNEEGG

SEQ ID NO: 261 - protein, 16 aa, synthetic construct
GGGGQDGNEEMGGGGG

SEQ ID NO: 262 - protein, 7 aa, synthetic construct
APFGSEM

SEQ ID NO: 263 - protein, 7 aa, synthetic construct
AWNGPYE

SEQ ID NO: 264 - protein, 10 aa, synthetic construct
AQDNGDTKTG

SEQ ID NO: 265 - protein, 8 aa, synthetic construct
AEAKETQG

SEQ ID NO: 266 - protein, 9 aa, synthetic construct
ATRREQVEG

SEQ ID NO: 267 - protein, 7 aa, synthetic construct
AQAPSQP

SEQ ID NO: 268 - protein, 10 aa, synthetic construct
ATRSRTRNDG

SEQ ID NO: 269 - protein, 10 aa, synthetic construct
ADVDAPDGLG

SEQ ID NO: 270 - protein, 9 aa, synthetic construct
AADISDPGG

SEQ ID NO: 271 - protein, 8 aa, synthetic construct
ALSVDPSG

SEQ ID NO: 272 - protein, 8 aa, synthetic construct
ARLSVDPG

SEQ ID NO: 273 - protein, 8 aa, synthetic construct
AVEAADRG

SEQ ID NO: 274 - protein, 12 aa, synthetic construct
GGPDGNEEMGGG

SEQ ID NO: 275 - protein, 12 aa, synthetic construct
GGFDGNEEMGGG

SEQ ID NO: 276 - protein, 12 aa, synthetic construct
GGGDGNEEMGGG

SEQ ID NO: 277 - protein, 12 aa, synthetic construct
GGEMDGEGQNGG

SEQ ID NO: 278 - protein, 14 aa, synthetic construct
GGGGGPDGNEEPGG

SEQ ID NO: 279 - protein, 14 aa, synthetic construct
GGGGSLDGNEEPGG

SEQ ID NO: 280 - protein, 14 aa, synthetic construct
GGGGALDGNEEPGG

SEQ ID NO: 281 - protein, 14 aa, synthetic construct
GGGGGLDGNEEPGG

SEQ ID NO: 282 - protein, 13 aa, synthetic construct
GGGALDGNEEPGG

SEQ ID NO: 283 - protein, 15 aa, synthetic construct
GGGGGPDGNEEPGGG

SEQ ID NO: 284 - protein, 14 aa, synthetic construct
GGSGALDGNEEPGG

SEQ ID NO: 285 - protein, 14 aa, synthetic construct
GGSGSLDGNEEPGG

SEQ ID NO: 286 - protein, 14 aa, synthetic construct
GGSGGPDGNEEPGG

SEQ ID NO: 287 - protein, 15 aa, synthetic construct
GGVRDGPDGNEEPGG

SEQ ID NO: 288 - protein, 16 aa, synthetic construct
GGSGGPDGNEEPGGGG

SEQ ID NO: 289 - protein, 14 aa, synthetic construct
GGGRGPDGNEEPGG

SEQ ID NO: 290 - protein, 14 aa, synthetic construct
GGSGGLDGNEEPGG

SEQ ID NO: 291 - protein, 14 aa, synthetic construct
GGGVGPDGNEEPGG

SEQ ID NO: 292 - protein, 14 aa, synthetic construct
GGGEGPDGNEEPGG

SEQ ID NO: 293 - protein, 14 aa, synthetic construct
GGGVALDGNEEPGG

SEQ ID NO: 294 - protein, 14 aa, synthetic construct
GGGRALDGNEEPGG

SEQ ID NO: 295 - protein, 14 aa, synthetic construct
GGYAGLDGNEEPGG

SEQ ID NO: 296 - protein, 14 aa, synthetic construct
GGAGGPDGNEEPGG

SEQ ID NO: 297 - protein, 14 aa, synthetic construct
GGRGGPDGNEEPGG

SEQ ID NO: 298 - protein, 15 aa, synthetic construct
GGGGPDGNEEPGGGG

SEQ ID NO: 299 - protein, 14 aa, synthetic construct
GGGEALDGNEEPGG

SEQ ID NO: 300 - protein, 14 aa, synthetic construct
GGDASLDGNEEPGG

SEQ ID NO: 301 - protein, 13 aa, synthetic construct
GGRDAPDGNEEGG

SEQ ID NO: 302 - protein, 10 aa, synthetic construct
SGGGGSGGVV

SEQ ID NO: 303 - protein, 16 aa, synthetic construct
SGGGGSGGGGSGGGGS

SEQ ID NO: 304 - protein, 17 aa, synthetic construct
SGGGGSGGGGSGGGGGS

SEQ ID NO: 305 - protein, 10 aa, synthetic construct
SGGGGSGGGS

SEQ ID NO: 306 - protein, 115 aa, synthetic construct
QVQLQESGGGLVQAGGSLRLSCAASGSIFSIASMGWYRQAPGKQRELVAVITSFSSTNYADSVKGRFTISRDNAKNTVYLQMNSLKPEDTGVYYCNARYFERTDWGQGTQVTVSS

SEQ ID NO: 307 - protein, 115 aa, synthetic construct
QVQLQESGGGLVQAGGSLRLSCAAPGSIFSIASMGWYRQAPGKQRELVAVITSFSSTNYADSVKGRFTISRDNAKNTVYLQMNSLKPEDTGVYYCNARYFERTDWGQGTQVTVSS

SEQ ID NO: 308 - protein, 115 aa, synthetic construct
QVQLQESGGGLVQAGGSLRLSCAASGSIFSIASMAWYRQAPGKQRELVAAITSFSSTNYADSVKGRFTISRDNAKNTVYLQMNSLKPEDTGVYYCNARYFERTDWGQGTQVTVSS

SEQ ID NO: 309 - protein, 115 aa, synthetic construct
QVQLQESGGGLVQAGGSLRLSCAASESIFSINVMAWHRQAPGKQRELVARITSGGSTNYADSVKGRFTISRDNAKNTVYLQMNSLKPEDTGVYYCGAYQGLYAYWGQGTQVTVSS

SEQ ID NO: 310 - protein, 115 aa, synthetic construct
QVQLQESGGGLVQAGGSLRLSCVASGSSFSITSMAWYRQAPGKQRDLVAAITSFGSTNYADSVKDRFTISRDNAKNTVYLQMNSLKPEDTAVYYCNGRVFDHVYWGQGTQVTVSS

SEQ ID NO: 311 - protein, 115 aa, synthetic construct
QVQLQESGGGLVQAGGSLKLSCAASSSIFSISSMSWYRQAPGKQRELVAAITTFDYTNYADSVKGRFTISRDNAKNMMYLQMNSLKPEDTAVYLCNARAFGRDYWGQGTQVTVSS

SEQ ID NO: 312 - protein, 115 aa, synthetic construct
QVQLQESGGGLVQAGGSLKLSCAASSSIFSISSMSWYRQAPGKQRELVAAITSFGSTNYADSVKGRFTISRDNAKNMMYLQMNSLKPEDTAVYRCNARTMGRDYWGQGTQVTVSS

SEQ ID NO: 313 - protein, 116 aa, synthetic construct
QVQLQESGGGLVQPGGSLRLSCAASGSTLNIKIMAWHRQAPGKQRELVATLTSGGNTNYADSVKGRFTISRDNAKNTVYLQMNSLQPEDTAVYYCGLWDGVGGAYWGRGTQVTVSS

SEQ ID NO: 314 - protein, 116 aa, synthetic construct
QVQLQESGGGLVQPGGSLRISCAASGSTLNIKIMAWHRQAPGKQRELVATLTSGGNTNYADSVKGRFTISRDNAKNTVYLQMNSLQPEDTAVYYCGLWDGVGGAYWGRGTQVTVSS

SEQ ID NO: 315 - protein, 116 aa, synthetic construct
QVQLQESGGGLVQAGGSLRLSCAASGSTFNIKTMAWHRQAPGNQRELVATLTSGGNTNYADSVKGRFTISRDNAKNTVYLQMNSLKPEDTAVYYCGLWNGVGGAYWGRGTQVTVSS

SEQ ID NO: 316 - protein, 115 aa, synthetic construct
QVQLQDGGGLVQPGGSLRLSCAASGSTFNIKLMAWHRQAPGNQRELVATLTSGGNTNYADSVKGRFTISRDNASNIVYLQMNSLKPEDTAVYYCGLWDGVGGAYWGRGTQVTVSS

SEQ ID NO: 317 - protein, 116 aa, synthetic construct
QVQLQESGGGLVQAGGSLRLSCAASGSTFNFKIMAWHRQAPGKQRELVASLTSEGLTNYRDSVKGRFTISRDNAKNTVYLQMNNLKPEDTAVYYCGLWDGVGGAYWGRGTQVTVSS

SEQ ID NO: 318 - protein, 114 aa, synthetic construct
QVQLQESGGGLVQPGGSLRLSCAASGFMFSSYSMSWYRQAPGKQRELVAAITTWGSTNYADSVKGRFTISRDNAKNTVWLQMNSLEPEDTAVYFCNARSWNNYWGQGTQVTVSS

SEQ ID NO: 319 - protein, 114 aa, synthetic construct
QVQLQESGGGLVQVGGSLRLSCAASGFMFSSYSMSWYRQAPGKQRELVAAITSYGSTNYADSVKGRFTISRDNAKNTVWLQMNSLKPEDTAVYFCNARSWNNYWGQGTQVTVSS

SEQ ID NO: 320 - protein, 114 aa, synthetic construct
QVQLQESGGGLVQPGGSLRLSCAASGFTFSSHSMSWYRQAPGKQRELVAAITTYGSTNYIDSVKGRFTISRDNTKNTVYLQMNSLKPEDTAVYFCNARSWNNYWGQGTQVTVSS

SEQ ID NO: 321 - protein, 117 aa, synthetic construct
QVQLQESGGGLVQAGGSLRLSCVASGSSFSHNTMGWYRQAPGKQRDLVARITTFGTTNYADSVKGRFTISRDNAKNTVYLQMNSLKPEDTAVYYCNGESFGRIWYNWGQGTQVTVSS

SEQ ID NO: 322 - protein, 116 aa, synthetic construct
QVQLQESGGGLVQAGASLRLTCTASGGRFSYATMGWSRQAPGKQREMVARITSSGFSTNYADSVKGRFTISRDNAKNAVYLQMDSLKPEDTAVYYCNAQHFGTDSWGQGTQVTVSS

SEQ ID NO: 323 - protein, 116 aa, synthetic construct
QVQLQESGGGLVQAGASLRLTCTASGSRFSYATMGWSRQAPGKQRELVARITSSGFSTNYADSVKGRFTISRDNAKNAVYLQMDSLKPEDTAVYYCNAQQFGTDSWGQGTQVTVSS

SEQ ID NO: 324 - protein, 117 aa, synthetic construct
QVQLQESGGGLVQAGGSLRLSCAASGSTFTSNVMGWHRQAPGKQRELVANMHSGGSTNYADSVKGRFTISRDNAKNIVYLQMNNLKIEDTAVYYCRWYGIQRAEGYWGQGTQVTVSS

SEQ ID NO: 325 - protein, 117 aa, synthetic construct
QVQLQESGGGLVVAGGSLRLSCAASGSSVSFLSMAWYRQAPGKKRELVAGISVDGSTNYADSVKGRFTVSRDNAKNTVYLQMNSLQPEDTAVYYCYAYRWVGRDTYWGQGTQVTVSS

SEQ ID NO: 326 - protein, 117 aa, synthetic construct
QVQLQESGGGLVVAGGSLRLSCAASGSSVSFLSMAWYRQAPGKKRELVAGISVDGSTNYADSVKGRFTISRDNAKNTVYLQMNSLQPEDTAVYYCYAYRWVGRDTYWGQGTQVTVSS

SEQ ID NO: 327 - protein, 117 aa, synthetic construct
QVQLQESGGGLVVSGGSLRLSCAASGSSVSFLSMAWYRQAPGKKRELVAGISVDGSTNYADSVKGRFTISRDNAKNTVYLQMNSLQPEDTAAYYCYAYRWVGRDTYWGQGTQVTVSS

SEQ ID NO: 328 - protein, 117 aa, synthetic construct
QVQLQESGGGLVVAGGSLRLSCAASGSSVSFLSMAWYRQAPGKKRELVAGISVDGSTNYADSVKGRFTISRDNAENTVYLQMNSLQPEDTAVYYCYAYRWEGRDTYWGQGTQVTVSS

SEQ ID NO: 329 - protein, 117 aa, synthetic construct
QVQLQESGGGLVVAGGSLRLSCAASGSSVSFLSMAWYRQAPGKKRELVAGISVDGSTNYADSVKGRFTISRDNAENTVYLQMNSLQPEDTAVYYCYAYRWEGRNTYWGQGTQVTVSS

SEQ ID NO: 330 - protein, 117 aa, synthetic construct
QVQLQESGGGLVQPGGSLRLSCAASGSSVSFLSMAWYRQAPGKKRELVAGISTDGSTNYVDSVKGRFTISRDNAKNTVYLQMNSLQPEDTAVYYCYAYRWVGRYTYWGQGTQVTVSS

SEQ ID NO: 331 - protein, 117 aa, synthetic construct
QVQLQESGGGLVVAGGSLRLSCAASGSSVSFLSMAWYRQAPGKKRELVAGISTDGTTNYVDSVKDRFTISRDNAKNTVYLQMNSLQPEDTAAYYCYAYRWVGRDTYWGQGTQVTVSS

SEQ ID NO: 332 - protein, 117 aa, synthetic construct
QVQLQESGGGLVVAGGSLRLSCAASGSSVSFLSIAWYRQAPGKKRELVAGISTDGTTNYVDSVKDRFTISRDNAKNTVYLQMNSLQPEDTAAYYCYAYRWVGRDTYWGQGTQVTVSS

SEQ ID NO: 333 - protein, 117 aa, synthetic construct
QVQLQESGGGLVQAGGSLTLSCAASGSSVSFLSMAWYRQAPGKKRELVAGISTDGTTNYVDSVKDRFTISRDNAKNTVYLQMNSLQPEDTAAYYCYAYRWVGRDTYWGQGTQVTVSS

SEQ ID NO: 334 - protein, 117 aa, synthetic construct
QVQLQESGGGLVQAGGSLRLSCAASGSSVSFLSMAWYRQAPGKKRELVAGISTDGSTNYADSVKGRFTISEGNAKNTVDLQMNSLQPEDTAVYYCYAYRWVDRYTYWGQGTQVTVSS

SEQ ID NO: 335 - protein, 117 aa, synthetic construct
QVQLQESGGGLVVAGGSLRLSCAASGSSVSFLSMAWYRQAPGKKRELVAGISTDGSTNYADSVKGRFTISEDNAKNTVDLQMNSLQPEDTAVYYCYAYRWIDRYTYWGQGTQVTVSS

SEQ ID NO: 336 - protein, 117 aa, synthetic construct
QVQLQESGGGLVQPGGSLRLSCAASGSSVSFLSMAWYRQAPGKKRELVAGISTDGSTNYADSVKGRFTISEDNAKNTVDLQMNSLQPEDTAVYYCYAYRWVDRYTYWGQGTQVTVSS

SEQ ID NO: 337 - protein, 117 aa, synthetic construct
QVQLQESGGGLVVAGGSLRLSCAASGSSVSFLSMAWYRQAPGKKRELVAGISSDGSTNYVDSVKGRFTISRDNAKNIVFLQMNSLQPQDTAVYYCYAYRWVGRDTYWGQGTQVTVSS

SEQ ID NO: 338 - protein, 117 aa, synthetic construct
QVQLQESGGGLVVAGGSLRLSCAASGSSVSFLSMAWYRQAPGKKRELVAGISADGSTDYIDSVKGRFTISRDSANNTMYLQMNSLQPEDTAVYYCYAYRWTTRYTYWGQGTQVTVSS

SEQ ID NO: 339 - protein, 117 aa, synthetic construct
QVQLQESGGGLVQPGGSLRLSCAASGFTLDYYAIGWYRQAPGKKRELVAGISSDGSTHYVDSVKGRFAISRDNAENTVYLQMNDLQPDDTAVYYCYAYRWVGGYTYWGQGTQVTVSS

SEQ ID NO: 340 - protein, 119 aa, synthetic construct
QVQLQESGGGLVQAGGSLRLSCVASGSTSSINAMGWYRRAPGKQRELVAGISSDGSKNYADSVKGRFTISRDNAKNTVYLQMNSLKPEDTAVYYCYYFRTVAASSMQYWGQGTQVTVSS

SEQ ID NO: 341 - protein, 119 aa, synthetic construct
QVQLQESGGGLVQAGGSLRLSCVASGSTSSINAMGWYRRAPGKQRELVAGISSDGSKVYADSVKGRFTISRDNAKNTVYLQMNSLKPEDTAVFYCYYFRTVSGSSMRYWGQGTQVTVSS

SEQ ID NO: 342 - protein, 118 aa, synthetic construct
QVQLQESGGGLVQAGGSLRLSCAASGITSSVYSMGWYRQAPGKQRELVAGSSSDGSTHYVDSVRGRFTISRDNAKNTVYLQMSSLKPEDTAVYYCYANRGFAGAPSYWGQGTQVTVSS

SEQ ID NO: 343 - protein, 114 aa, synthetic construct
QVQLQESGGGLVQAGGSLRLSCAASGRTSMFNSMGWHRQAPGKQRELVAIIRSGGSSNYADTVKGRFTISRDNTKNTVYLQMNDLKPEDTAVYYCFYYFQSSYWGQGTQVTVSS

SEQ ID NO: 344 - protein, 114 aa, synthetic construct
QVQLQESGGGLVQAGGSLRLSCAASGRTSMVNSMGWHRQAPGKQRELVALITSGGSSNYADTVKGRFTISRDNTKNTVYLQMNDLKPEDTAVYYCFYYFQSSYWGQGTQVTVSS

SEQ ID NO: 345 - protein, 119 aa, synthetic construct
QVQLQESGGGLVQAGGSLRLSCTASGSIFSIAVMGWYRQVPGKRREWVATIFDGSYTNYADSVKGRFTISRDNARNKVYLQMNNLKPEDTAVYYCQTHWTQGSVPKESWGQGTQVTVSS

SEQ ID NO: 346 - protein, 115 aa, synthetic construct
QVQLQESGGGLVQAGGSLRLSCVASSGIFSDMSMVWYRQAPGKQRELVASITTFGSTNYADPVKGRFTISRDNAKNTVYLQMNSLKPEDTAVYYCSGRSYSSDYWGRGTQVTVSS

SEQ ID NO: 347 - protein, 115 aa, synthetic construct
QVQLQESGGGLVQAGGSLRLSCVASGSISSIIVMGWSRQAPGKQRESVATITRDGTRNYADSLKGRFTISRDNAKNTSYLQINSLKPEDTAVYSCYARYGDINYWGKGTQVTVSS

SEQ ID NO: 348 - protein, 115 aa, synthetic construct
QVQLQESGGGLVQAGGSLRLSCVASGSISSIIVMGWSRQAPGKQRESLATISRGGTRTYADSVKGRFTISRDNAKNTSYLQMNSLKPEDTAVYSCYARYGDINYWGKGTQVTVSS

SEQ ID NO: 349 - protein, 114 aa, synthetic construct
QVQLQESGGGLVQAGGSLRLSCVASGSIFTTNSMGWHRQGPGKQRELVALIGSAGSTKYADSVKGRFTISRDNAKNTVSLQMDSLKPEDTAVYYCFYYDSRSYWGQGTQVTVSS

SEQ ID NO: 350 - protein, 114 aa, synthetic construct
QVQLQESGGGMVQPGGSLRLSCAASGSREISTMGWHRQAPGKQRELAARITSGGITKYADSVKGRFTISRDNAKKTVYLQMNSLKSEDTAVYYCFAYDNINAYWGQGTQVTVSS

SEQ ID NO: 351 - protein, 114 aa, synthetic construct
QVQLQESGGGWVQAGGSLRLSCAASGSREISTMGWHRQAPGKQRELAARITSGGITKYADSVKGRFTISRDNAKKTVYLQMNSLKSEDTAVYYCFAYDNINAYWGQGTQVTVSS

SEQ ID NO: 352 - protein, 114 aa, synthetic construct
QVQLQESGGGWVQAGGSLRLSCTASGSREISTMGWHRQAPGKQRELAARITSGGITKYADSVKGRFTISRDNAKKTVYLQMDSLKSEDTAVYYCFAYDNINAYWGQGTQVTVSS

SEQ ID NO: 353 - protein, 114 aa, synthetic construct
QVQLQESGGGSVQAGRSLGLSCAASGSREISTMGWHRQAPGKQRELAARITSGGITKYADSVKGRFTISRDNAKKTVYLQMNSLKSEDTAVYYCFAYDNINAYWGQGTQVTVSS

SEQ ID NO: 354 - protein, 115 aa, synthetic construct
QVQLQESGGGLVQAGGSLRLSCTASGSIFRGAAMYWHRQAPGKQRELVAAITTSGNTSYADSVKGRFTISRDNAKNTMYLQIISLKPEDTAVYYCAFWIAGKAYWGQGTQVTVSS

SEQ ID NO: 355 - protein, 115 aa, synthetic construct
QVQLQESGGGLVQPGGSLRLSCAASGSISSFNFMSWHRQAPGKERELAGVITRGGATNYADSVKGRFTISRDNVKNTVYLQMNGLKPEDTAVYYCHGRSQLGSTWGQGTQVTVSS

SEQ ID NO: 356 - protein, 116 aa, synthetic construct
QVQLQESGGGLVQAGGSLRLSCLASGTIFTASTMGWHRQPPGKQRELVASIAGDGRTNYAESTEGRFTISRDDAKNTMYLQMNSLKPEDTAVYYCYAYYLDTYAYWGQGTQVTVSS

SEQ ID NO: 357 - protein, 115 aa, synthetic construct
EVQLVESGGGLVQPGGSLTLSCAASGSIFSIASMGWYRQAPGKQRELVAVITSFSSTNYADSVKGRFTISRDNAKNSVYLQMNSLRAEDTAVYYCNARYFERTDWGQGTLVTVSS

SEQ ID NO: 358 - protein, 114 aa, synthetic construct
EVQLVESGGGLVQPGGSLTLSCAASGRTSMFNSMGWHRQAPGKQRELVAIIRSGGSSNYADTVKGRFTISRDNAKNSVYLQMNSLRAEDTAVYYCFYYFQSSYWGQGTLVTVSS

SEQ ID NO: 359 - protein, 117 aa, synthetic construct
EVQLVESGGGLVQPGGSLTLSCAASGSSVSFLSMAWYRQAPGKKRELVAGISVDGSTNYADSVKGRFTISRDNAKNSVYLQMNSLRAEDTAVYYCYAYRWVGRDTYWGQGTLVTVSS

SEQ ID NO: 360 - protein, 115 aa, synthetic construct
EVQLVESGGGLVQPGGSLTLSCTASGSIFRGAAMYWHRQAPGKQRELVAAITTSGNTSYADSVKGRFTISRDNAKNSMYLQMNSLRAEDTAVYYCAFWIAGKAYWGQGTLVTVSS

SEQ ID NO: 361 - protein, 116 aa, synthetic construct
EVQLVESGGGLVQPGGSLTLSCAASGSTFNIKTMAWHRQAPGNQRELVATLTSGGNTNYADSVKGRFTISRDNAKNSVYLQMNSLRAEDTAVYYCGLWNGVGGAYWGQGTLVTVSS

SEQ ID NO: 362 - protein, 116 aa, synthetic construct
EVQLVESGGGLVQPGGSLTLSCAASGSTLNIKIMAWHRQAPGKQRELVATLTSGGNTNYADSVKGRFTISRDNAKNSVYLQMNSLRAEDTAVYYCGLWDGVGGAYWGQGTLVTVSS

SEQ ID NO: 363 - protein, 115 aa, synthetic construct
EVQLVESGGGLVQPGGSLTLSCAASSSIFSISSMSWYRQAPGKQRELVAAITTFDYTNYADSVKGRFTISRDNAKNSMYLQMNSLRAEDTAVYYCNARAFGRDYWGQGTLVTVSS

SEQ ID NO: 364 - protein, 119 aa, synthetic construct
EVQLVESGGGLVQPGGSLTLSCVASGSTSSINAMGWYRRAPGKQRELVAGISSDGSKNYADSVKGRFTISRDNAKNSVYLQMNSLRAEDTAVYYCYYFRTVAASSMQYWGQGTLVTVSS

SEQ ID NO: 365 - protein, 114 aa, synthetic construct
EVQLVESGGGLVQPGGSLTLSCAASGFMFSSYSMSWYRQAPGKQRELVAAITSYGSTNYADSVKGRFTISRDNAKNSVYLQMNSLRAEDTAVYYCNARSWNNYWGQGTLVTVSS

SEQ ID NO: 366 - protein, 114 aa, synthetic construct
EVQLVESGGGLVQPGGSLTLSCAASGSVSMFNSMGWHRQPPGKQRELVAIITSGGSSNYADTVKGRFTISRDNAKNSVYLQMNSLRAEDTAVYYCFYYFQSSYWGQGTLVTVSS

SEQ ID NO: 367 - protein, 116 aa, synthetic construct
EVQLVESGGGLVQPGGSLTLSCTASGGRFSYATMGWSRQAPGKQREMVARITSSGFSTNYADSVKGRFTISRDNAKNSVYLQMNSLRAEDTAVYYCNAQHFGTDSWGQGTLVTVSS

SEQ ID NO: 368 - protein, 115 aa, synthetic construct
EVQLVESGGGLVQPGGSLTLSCAASGSISSFNFMSWHRQAPGKERELAGVITRGGATNYADSVKGRFTISRDNAKNSVYLQMNSLRAEDTAVYYCHGRSQLGSTWGQGTLVTVSS

SEQ ID NO: 369 - protein, 115 aa, synthetic construct
EVQLVESGGGLVQPGGSLTLSCAASESIFSINVMAWHRQAPGKQRELVARITSGGSTNYADSVKGRFTISRDNAKNSVYLQMNSLRAEDTAVYYCGAYQGLYAYWGQGTLVTVSS

SEQ ID NO: 370 - protein, 114 aa, synthetic construct
EVQLVESGGGLVQPGGSLTLSCAASGSREISTMGWHRQAPGKQRELAARITSGGITKYADSVKGRFTISRDNAKNSVYLQMNSLRAEDTAVYYCFAYDNINAYWGQGTLVTVSS

SEQ ID NO: 371 - protein, 119 aa, synthetic construct
EVQLVESGGGLVQPGGSLTLSCTASGSIFSIAVMGWYRQVPGKRREWVATIFDGSYTNYADSVKGRFTISRDNAKNSVYLQMNSLRAEDTAVYYCQTHWTQGSVPKESWGQGTLVTVSS

SEQ ID NO: 372 - protein, 119 aa, synthetic construct
EVQLVESGGGLVQPGGSLTLSCVASGSTSSINAMGWYRRAPGKQRELVAGISSDGSKVYADSVKGRFTISRDNAKNSVYLQMNSLRAEDTAVYYCYYFRTVSGSSMRYWGQGTLVTVSS

SEQ ID NO: 373 - protein, 115 aa, synthetic construct
EVQLVESGGGLVQPGGSLTLSCVASSGIFSDMSMVWYRQAPGKQRELVASITTFGSTNYADPVKGRFTISRDNAKNSVYLQMNSLRAEDTAVYYCSGRSYSSDYWGQGTLVTVSS

SEQ ID NO: 374 - protein, 114 aa, synthetic construct
EVQLVESGGGLVQPGGSLTLSCAASGFMFSSYSMSWYRQAPGKQRELVAAITTWGSTNYADSVKGRFTISRDNAKNSVYLQMNSLRAEDTAVYYCNARSWNNYWGQGTLVTVSS

SEQ ID NO: 375 - protein, 116 aa, synthetic construct
EVQLVESGGGLVQPGGSLTLSCTASGSRFSYATMGWSRQAPGKQRELVARITSSGFSTNYADSVKGRFTISRDNAKNSVYLQMNSLRAEDTAVYYCNAQQFGTDSWGQGTLVTVSS

SEQ ID NO: 376 - protein, 117 aa, synthetic construct
EVQLVESGGGLVQPGGSLTLSCAASGSTFTSNVMGWHRQAPGKQRELVANMHSGGSTNYADSVKGRFTISRDNAKNSVYLQMNSLRAEDTAVYYCRWYGIQRAEGYWGQGTLVTVSS

SEQ ID NO: 377 - protein, 117 aa, synthetic construct
EVQLVESGGGLVQPGGSLTLSCAASGSSVSFLSMAWYRQAPGKKRELVAGISTDGSTNYVDSVKGRFTISRDNAKNSVYLQMNSLRAEDTAVYYCYAYRWVGRYTYWGQGTLVTVSS

SEQ ID NO: 378 - protein, 115 aa, synthetic construct
EVQLVESGGGLVQPGGSLTLSCVASGSISSIIVMGWSRQAPGKQRESVATITRDGTRNYADSLKGRFTISRDNAKNSSYLQMNSLRAEDTAVYYCYARYGDINYWGQGTLVTVSS

SEQ ID NO: 379 - protein, 117 aa, synthetic construct
EVQLVESGGGLVQPGGSLTLSCAASGSSVSFLSMAWYRQAPGKKRELVAGISADGSTDYIDSVKGRFTISRDNAKNSVYLQMNSLRAEDTAVYYCYAYRWTTRYTYWGQGTLVTVSS

SEQ ID NO: 380 - protein, 116 aa, synthetic construct
EVQLVESGGGLVQPGGSLTLSCLASGTIFTASTMGWHRQPPGKQRELVASIAGDGRTNYAESTEGRFTISRDNAKNSMYLQMNSLRAEDTAVYYCYAYYLDTYAYWGQGTLVTVSS

SEQ ID NO: 381 - protein, 117 aa, synthetic construct
EVQLVESGGGLVQPGGSLTLSCAASGSSVSFLSMAWYRQAPGKKRELVAGISVDGSTNYADSVKGRFTISRDNAKNSVYLQMNSLRAEDTAVYYCYAYRWEGRNTYWGQGTLVTVSS

SEQ ID NO: 382 - protein, 117 aa, synthetic construct
EVQLVESGGGLVQPGGSLTLSCVASGSSFSHNTMGWYRQAPGKQRDLVARITTFGTTNYADSVKGRFTISRDNAKNSVYLQMNSLRAEDTAVYYCNGESFGRIWYNWGQGTLVTVSS

SEQ ID NO: 383 - protein, 115 aa, synthetic construct
EVQLVESGGGLVQPGGSLTLSCVASGSISSIIVMGWSRQAPGKQRESLATISRGGTRTYADSVKGRFTISRDNAKNSSYLQMNSLRAEDTAVYYCYARYGDINYWGQGTLVTVSS

SEQ ID NO: 384 - protein, 115 aa, synthetic construct
EVQLVESGGGLVQPGGSLTLSCVASGSSFSITSMAWYRQAPGKQRDLVAAITSFGSTNYADSVKDRFTISRDNAKNSVYLQMNSLRAEDTAVYYCNGRVFDHVYWGQGTLVTVSS

SEQ ID NO: 385 - protein, 114 aa, synthetic construct
EVQLVESGGGLVQPGGSLTLSCAASGRTSMVNSMGWHRQAPGKQRELVALITSGGSSNYADTVKGRFTISRDNAKNSVYLQMNSLRAEDTAVYYCFYYFQSSYWGQGTLVTVSS

SEQ ID NO: 386 - protein, 116 aa, synthetic construct
EVQLVESGGGLVQPGGSLTLSCAASGSTFNFKIMAWHRQAPGKQRELVASLTSEGLTNYRDSVKGRFTISRDNAKNSVYLQMNSLRAEDTAVYYCGLWDGVGGAYWGQGTLVTVSS

SEQ ID NO: 387 - protein, 117 aa, synthetic construct
EVQLVESGGGLVQPGGSLTLSCAASGFTLDYYAIGWYRQAPGKKRELVAGISSDGSTHYVDSVKGRFTISRDNAKNSVYLQMNSLRAEDTAVYYCYAYRWVGGYTYWGQGTLVTVSS

SEQ ID NO: 388 - protein, 114 aa, synthetic construct
EVQLVESGGGLVQPGGSLTLSCVASGSIFTTNSMGWHRQGPGKQRELVALIGSAGSTKYADSVKGRFTISRDNAKNSVYLQMNSLRAEDTAVYYCFYYDSRSYWGQGTLVTVSS

SEQ ID NO: 389 - protein, 118 aa, synthetic construct
EVQLVESGGGLVQPGGSLTLSCAASGITSSVYSMGWYRQAPGKQRELVAGSSSDGSTHYVDSVRGRFTISRDNAKNSVYLQMNSLRAEDTAVYYCYANRGFAGAPSYWGQGTLVTVSS

SEQ ID NO: 390 - protein, 115 aa, synthetic construct
EVQLVESGGGLVQPGGSLTLSCAASSSIFSISSMSWYRQAPGKQRELVAAITSFGSTNYADSVKGRFTISRDNAKNSMYLQMNSLRAEDTAVYYCNARTMGRDYWGQGTLVTVSS

SEQ ID NO: 391 - protein, 119 aa, synthetic construct
EVQLVESGGGLVQPGGSLTLSCVASGFTSSINAMGWYRRAPGKQRELVAGISSDGSFVYADSVKGRFTISRDNAKNSVYLQMNSLRAEDTAVYYCYYFRHVSGSSMRYWGQGTLVTVSS

SEQ ID NO: 392 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTVSGSSSRYWGQGTLVTVSS

SEQ ID NO: 393 - protein, 119 aa, synthetic construct
EVQLVESGGGLVQPGGSLTLSCVASGSTSSINAMGWYRRAPGKQRELVAGISSDGSKVYEDSVKGRFTISRDNAKNSVYLQMNSLRAEDTAVYYCYYFRTVSGSSMRYWGQGTLVTVSS

SEQ ID NO: 394 - protein, 119 aa, synthetic construct
EVQLVESGGGLVQPGGSLTLSCVASGSPSSINAMGWYRRAPGKQRELSAGISSDGSKVYADSVKGRFTISRDNAKNSVYLQMNSLRAEDTAVYYCYYFRTVRGSSMSYWGQGTLVTVSS

SEQ ID NO: 395 - protein, 119 aa, synthetic construct
EVQLVESGGGLVQPGGSLTLSCVASGSTSSINAMGWYRRAPGKQRELAAGISSDGSSVYADSVKGRFTISRDNAKNSVYLQMNSLRAEDTAVYYCYYFRTVSGSSKRYWGQGTLVTVSS

SEQ ID NO: 396 - protein, 119 aa, synthetic construct
EVQLVESGGGLVQPGGSLTLSCVASGSISSINAMGWYRRAPGKQRELVAGISSDGSKVYADSVKGRFTISRDNAKNSVYLQMNSLRAEDTAVYYCYYFRMVSGSSMRYWGQGTLVTVSS

SEQ ID NO: 397 - protein, 119 aa, synthetic construct
EVQLVESGGGLVQPGGSLTLSCVASGSTSSINAMAWYRRAPGKQRELVAGISSDGSKLYADSVKGRFTISRDNAKNSVYLQMNSLRAEDTAVYYCYYFRTVQGSSMRYWGQGTLVTVSS

SEQ ID NO: 398 - protein, 119 aa, synthetic construct
EVQLVESGGGLVQPGGSLTLSCVASGSTSSINAYGWYRRAPGKQRELVAGISSDGSKVYADSVKGRFTISRDNAKNSVYLQMNSLRAEDTAVYYCYYFRTVYGSSMRYWGQGTLVTVSS

SEQ ID NO: 399 - protein, 119 aa, synthetic construct
EVQLVESGGGLVQPGGSLTLSCVASGSTSSINAIGWYRRAPGKQRELVAGISSDGSKVYIDSVKGRFTISRDNAKNSVYLQMNSLRAEDTAVYYCYYFRTVSGSSYRYWGQGTLVTVSS

SEQ ID NO: 400 - protein, 119 aa, synthetic construct
EVQLVESGGGLVQPGGSLTLSCVASGSTSSINAMAWYRRAPGKQRELVAGISSDGSKVYSDSVKGRFTISRDNAKNSVYLQMNSLRAEDTAVYYCYYFRTVLGSSMRYWGQGTLVTVSS

SEQ ID NO: 401 - protein, 119 aa, synthetic construct
EVQLVESGGGLVQPGGSLTLSCVASGSTSIINAMGWYRRAPGKQRELAAGISSDGSKVIADSVKGRFTISRDNAKNSVYLQMNSLRAEDTAVYYCYYFRRVSGSSMRYWGQGTLVTVSS

SEQ ID NO: 402 - protein, 119 aa, synthetic construct
EVQLVESGGGLVQPGGSLTLSCVASGSTSSINAMGWYRRAPGKQRELVAGISSDGSKIYADSVKGRFTISRDNAKNSVYLQMNSLRAEDTAVYYCYYFRTVSGSSMRYWGQGTLVTVSS

SEQ ID NO: 403 - protein, 119 aa, synthetic construct
EVQLVESGGGLVQPGGSLTLSCVASGKTSSINAMAWYRRAPGKQRELVAGISSDGSKVYTDSVKGRFTISRDNAKNSVYLQMNSLRAEDTAVYYCYYFRTVSGSSARYWGQGTLVTVSS

SEQ ID NO: 404 - protein, 119 aa, synthetic construct
EVQLVESGGGLVQPGGSLTLSCVASGTTSSINAMGWYRRAPGKQRELVAGISSDGSLVYADSVKGRFTISRDNAKNSVYLQMNSLRAEDTAVYYCYYFRIVRGSSMRYWGQGTLVTVSS

SEQ ID NO: 405 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYYRTVSGSSMRYWGQGTLVTVSS

SEQ ID NO: 406 - protein, 119 aa, synthetic construct
EVQLVESGGGLVQPGGSLTLSCVASGSGSSINAMGWYRRAPGKQRELVAGISSDGSKVYSDSVKGRFTISRDNAKNSVYLQMNSLRAEDTAVYYCYYFRHVSGSSMRYWGQGTLVTVSS

SEQ ID NO: 407 - protein, 119 aa, synthetic construct
EVQLVESGGGLVQPGGSLTLSCVASGTTSSINAMGWYRRAPGKQRELVAGISSDGSKVYVDSVKGRFTISRDNAKNSVYLQMNSLRAEDTAVYYCYYFRFVSGSSMRYWGQGTLVTVSS

SEQ ID NO: 408 - protein, 119 aa, synthetic construct
EVQLVESGGGLVQPGGSLTLSCVASGSTSSINAMAWYRRAPGKQRELVAGISSDGSKVYVDSVKGRFTISRDNAKNSVYLQMNSLRAEDTAVYYCYYFRTVSGSSMRYWGQGTLVTVSS

SEQ ID NO: 409 - protein, 119 aa, synthetic construct
EVQLVESGGGLVQPGGSLTLSCVASGSTSRINAMGWYRRAPGKQRELVAGISSDGSKVYADSVKGRFTISRDNAKNSVYLQMNSLRAEDTAVYYCYYFRTKSGSSMRYWGQGTLVTVSS

SEQ ID NO: 410 - protein, 119 aa, synthetic construct
EVQLVESGGGLVQPGGSLTLSCVASGSTSRINAMGWYRRAPGKQRELVAGISSDGSKVYADSVKGRFTISRDNAKNSVYLQMNSLRAEDTAVYYCYYFRTVYGSSMRYWGQGTLVTVSS

SEQ ID NO: 411 - protein, 119 aa, synthetic construct
EVQLVESGGGLVQPGGSLTLSCVASGSTSSINAMGWYRRAPGKQRELVAGISSDGSKVYRDSVKGRFTISRDNAKNSVYLQMNSLRAEDTAVYYCYYFRTVSGSSMGYWGQGTLVTVSS

SEQ ID NO: 412 - protein, 119 aa, synthetic construct
EVQLVESGGGLVQPGGSLTLSCVASGSTSSINAMAWYRRAPGKQRELVAGISSDGSKVYSDSVKGRFTISRDNAKNSVYLQMNSLRAEDTAVYYCYYFRTVSGSSMRSWGQGTLVTVSS

SEQ ID NO: 413 - protein, 119 aa, synthetic construct
EVQLVESGGGLVQPGGSLTLSCVASGSTSSINAMAWYRRAPGKQRELVAGISSDNSKVYADSVKGRFTISRDNAKNSVYLQMNSLRAEDTAVYYCYYFRTVGGSSMRYWGQGTLVTVSS

SEQ ID NO: 414 - protein, 119 aa, synthetic construct
EVQLVESGGGLVQPGGSLTLSCVASGNTSSINAMAWYRRAPGKQRELVAGISSDGSKVYADSVKGRFTISRDNAKNSVYLQMNSLRAEDTAVYYCYYFRTVSGSSMRYWGQGTLVTVSS

SEQ ID NO: 415 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTVSGSHMRYWGQGTLVTVSS

SEQ ID NO: 416 - protein, 119 aa, synthetic construct
EVQLVESGGGLVQPGGSLTLSCVASGSTSIINAMGWYRRAPGKQRELVAGISSDGSKVYEDSVKGRFTISRDNAKNSVYLQMNSLRAEDTAVYYCYYFRAVSGSSMRYWGQGTLVTVSS

SEQ ID NO: 417 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTVSGSSMRYWGQGTLVTVSS

SEQ ID NO: 418 - protein, 119 aa, synthetic construct
EVQLVESGGGLVQPGGSLTLSCVASGSTSRINAMGWYRRAPGKQRELPAGISSDGSKVYAVSVKGRFTISRDNAKNSVYLQMNSLRAEDTAVYYCYYFRTVSGSPMRYWGQGTLVTVSS

SEQ ID NO: 419 - protein, 119 aa, synthetic construct
EVQLVESGGGLVQPGGSLTLSCVASGSTSRINAMGWYRRAPGKQRELVAGVSSDGSKVYADSVKGRFTISRDNAKNSVYLQMNSLRAEDTAVYYCYYFRTVSGSSMSYWGQGTLVTVSS

SEQ ID NO: 420 - protein, 119 aa, synthetic construct
EVQLVESGGGLVQPGGSLTLSCVASGTTSSINAMGWYRRAPGKQRELVAGISSDGSKVYEDSVKGRFTISRDNAKNSVYLQMNSLRAEDTAVYYCYYFRTVSGSSMRYWGQGTLVTVSS

SEQ ID NO: 421 - protein, 119 aa, synthetic construct
EVQLVESGGGLVQPGGSLTLSCVASGITSSINAMGWYRRAPGKQRELVAGISSDGSKVYAGSVKGRFTISRDNAKNSVYLQMNSLRAEDTAVYYCYYFRTVRGSSMRYWGQGTLVTVSS

SEQ ID NO: 422 - protein, 119 aa, synthetic construct
EVQLVESGGGLVQPGGSLTLSCVASGSTSDINAMGWYRRAPGKQRELVAGISSDKSKVYADSVKGRFTISRDNAKNSVYLQMNSLRAEDTAVYYCYYFRTVRGSSMRYWGQGTLVTVSS

SEQ ID NO: 423 - protein, 119 aa, synthetic construct
EVQLVESGGGLVQPGGSLTLSCVASGSTSSINAMAWYRRAPGKQRELVAGISSNGSKVYADSVKGRFTISRDNAKNSVYLQMNSLRAEDTAVYYCYYFRQVSGSSMRYWGQGTLVTVSS

SEQ ID NO: 424 - protein, 119 aa, synthetic construct
EVQLVESGGGLVQPGGSLTLSCVASGSTSSINAIGWYRRAPGKQRELVAGISSDGSKVLADSVKGRFTISRDNAKNSVYLQMNSLRAEDTAVYYCYYFRIVSGSSMGYWGQGTLVTVSS

SEQ ID NO: 425 - protein, 119 aa, synthetic construct
EVQLVESGGGLVQPGGSLTLSCVASGSTSSKNAMGWYRRAPGKQRELVAGISSDGSKVYADSVKGRFTISRDNAKNSVYLQMNSLRAEDTAVYYCYYFRTVSGASMRYWGQGTLVTVSS

SEQ ID NO: 426 - protein, 119 aa, synthetic construct
EVQLVESGGGLVQPGGSLTLSCVASGSTSSINAMGWYRRAPGKQRELVAGISSDNSKVYADSVKGRFTISRDNAKNSVYLQMNSLRAEDTAVYYCYYFRTVHGSSMRYWGQGTLVTVSS

SEQ ID NO: 427 - protein, 119 aa, synthetic construct
EVQLVESGGGLVQPGGSLTLSCVASGLTSSINAMGWYRRAPGKQRELVAGISSDGSKVYADSVKGRFTISRDNAKNSVYLQMNSLRAEDTAVYYCYYFRMVSGSSMRYWGQGTLVTVSS

SEQ ID NO: 428 - protein, 119 aa, synthetic construct
EVQLVESGGGLVQPGGSLTLSCVASGSTSSINAMAWYRRAPGKQRELVAGISSDGSKVYTDSVKGRFTISRDNAKNSVYLQMNSLRAEDTAVYYCYYFRTISGSSMRYWGQGTLVTVSS

SEQ ID NO: 429 - protein, 119 aa, synthetic construct
EVQLVESGGGLVQPGGSLTLSCVASGSTSSNNAMAWYRRAPGKQRELVAGISSDGSKVYTDSVKGRFTISRDNAKNSVYLQMNSLRAEDTAVYYCYYFRTRSGSSMRYWGQGTLVTVSS

SEQ ID NO: 430 - protein, 119 aa, synthetic construct
EVQLVESGGGLVQPGGSLTLSCVASGSTSSINAMAWYRRAPGKQRELVAGISSDNSKVYADSVKGRFTISRDNAKNSVYLQMNSLRAEDTAVYYCYYFRTVSGHSMRYWGQGTLVTVSS

SEQ ID NO: 431 - protein, 119 aa, synthetic construct
EVQLVESGGGLVQPGGSLTLSCVASGSTSHINAMGWYRRAPGKQRELVAGISSDGSRVYADSVKGRFTISRDNAKNSVYLQMNSLRAEDTAVYYCYYFRTVSGGSMRYWGQGTLVTVSS

SEQ ID NO: 432 - protein, 119 aa, synthetic construct
EVQLVESGGGLVQPGGSLTLSCVASGQTSSINAMGWYRRAPGKQRELVAGISSDGSQVYADSVKGRFTISRDNAKNSVYLQMNSLRAEDTAVYYCYYFRTKSGSSMRYWGQGTLVTVSS

SEQ ID NO: 433 - protein, 119 aa, synthetic construct
EVQLVESGGGLVQPGGSLTLSCVASGSTSRINGMGWYRRAPGKQRELPAGISSDGSKAYADSVKGRFTISRDNAKNSVYLQMNSLRAEDTAVYYCYYFRTASGTSMRYWGQGTLVTVSS

SEQ ID NO: 434 - protein, 119 aa, synthetic construct
EVQLVESGGGLVQPGGSLTLSCVASGSTSVINAMAWYRRAPGKQRELAAGISSDGSKVYAKSAKGRFTISRDNAKNSVYLQMNSLRAEDTAVYYCYYFNTVSGSSMRYWGQGTLVTVSS

SEQ ID NO: 435 - protein, 119 aa, synthetic construct
EVQLVESGGGLVQPGGSLTLSCVASGSTSSINAMAWYRRAPGKQRELVAGISSDGSKVYNDSVKGRFTISRDNAKNSVYLQMNSLRAEDTAVYYCYYFRTVRGSSQRYWGQGTLVTVSS

SEQ ID NO: 436 - protein, 119 aa, synthetic construct
EVQLVESGGGLVQPGGSLTLSCVASGKTSSINAMGWYRRAPGKQRELVAGISSDGSKVIADSVKGRFTISRDNAKNSVYLQMNSLRAEDTAVYYCYYFRTVLGSSMRYWGQGTLVTVSS

SEQ ID NO: 437 - protein, 119 aa, synthetic construct
EVQLVESGGGLVQPGGSLTLSCVASGSTSSINAMAWYRRAPGKQRELVAGISSDGSKVYTDSVKGRFTISRDNAKNSVYLQMNSLRAEDTAVYYCYYFRTRSGSSMRYWGQGTLVTVSS

SEQ ID NO: 438 - protein, 119 aa, synthetic construct
EVQLVESGGGLVQPGGSLTLSCVASGSVSSINAMGWYRRAPGKQRELVAGISSDGSKVYIDSVKGRFTISRDNAKNSVYLQMNSLRAEDTAVYYCYYFRTVSGLSMRYWGQGTLVTVSS

SEQ ID NO: 439 - protein, 119 aa, synthetic construct
EVQLVESGGGLVQPGGSLTLSCVASGNTSSINAMGWYRRAPGKQRELVAGISSDGSKVYYDSVKGRFTISRDNAKNSVYLQMNSLRAEDTAVYYCYYFRTVRGSSQRYWGQGTLVTVSS

SEQ ID NO: 440 - protein, 119 aa, synthetic construct
EVQLVESGGGLVQPGGSLTLSCVASGSTSSTNAMGWYRRAPGKQRELVAGISSDGSKVYVDSVKGRFTISRDNAKNSVYLQMNSLRAEDTAVYYCYYFRTVSGSSMVYWGQGTLVTVSS

SEQ ID NO: 441 - protein, 119 aa, synthetic construct
EVQLVESGGGLVQPGGSLTLSCVASGSTSSINAMGWYRRAPGKQRELVAGISSDGSKVYGDSVKGRFTISRDNAKNSVYLQMNSLRAEDTAVYYCYYFRTVSRSSMRYWGQGTLVTVSS

SEQ ID NO: 442 - protein, 119 aa, synthetic construct
EVQLVESGGGLVQPGGSLTLSCVASGSTSSINAMAWYRRAPGKQRELAAGISSDQSKVYADSAKGRFTISRDNAKNSVYLQMNSLRAEDTAVYYCYYFRTVSGSSMSYWGQGTLVTVSS

SEQ ID NO: 443 - protein, 119 aa, synthetic construct
EVQLVESGGGLVQPGGSLTLSCVASGGTSSINAMGWYRRAPGKQRELVAGISSDGSKVYSDSVKGRFTISRDNAKNSVYLQMNSLRAEDTAVYYCYYFRTVSGSSARYWGQGTLVTVSS

SEQ ID NO: 444 - protein, 119 aa, synthetic construct
EVQLVESGGGLVQPGGSLTLSCVASGSTRSINAMGWYRRAPGKQRELVAGISSDGSKVYADSVKGRFTISRDNAKNSVYLQMNSLRAEDTAVYYCYYFHTVSGSSMRYWGQGTLVTVSS

SEQ ID NO: 445 - protein, 119 aa, synthetic construct
EVQLVESGGGLVQPGGSLTLSCVASGTTSSINAMGWYRRAPGKQRELVAGISSDGSKVIADSVKGRFTISRDNAKNSVYLQMNSLRAEDTAVYYCYYFRTVLGSSMRYWGQGTLVTVSS

SEQ ID NO: 446 - protein, 119 aa, synthetic construct
EVQLVESGGGLVQPGGSLTLSCVASGSTSSINAMGWYRRAPGKQRELVAGISSDGSKVDADSVKGRFTISRDNAKNSVYLQMNSLRAEDTAVYYCYYFRTVSGSSMRYWGQGTLVTVSS

SEQ ID NO: 447 - protein, 119 aa, synthetic construct
EVQLVESGGGLVQPGGSLTLSCVASGSTSSINAMAWYRRAPGKQRELVAGISSDGSKVYKDSVKGRFTISRDNAKNSVYLQMNSLRAEDTAVYYCYYFRNVSGSSMRYWGQGTLVTVSS

SEQ ID NO: 448 - protein, 119 aa, synthetic construct
EVQLVESGGGLVQPGGSLTLSCVASGNTSSINAMGWYRRAPGKQRELVAGISSNGSKVYADSVKGRFTISRDNAKNSVYLQMNSLRAEDTAVYYCYYFRTVTGSSMRYWGQGTLVTVSS

SEQ ID NO: 449 - protein, 119 aa, synthetic construct
EVQLVESGGGLVQPGGSLTLSCVASGSTSRINAMGWYRRAPGKQRELVAGISSDGSKVYKDSVKGRFTISRDNAKNSVYLQMNSLRAEDTAVYYCYYFRTVSGSSMRYWGQGTLVTVSS

SEQ ID NO: 450 - protein, 119 aa, synthetic construct
EVQLVESGGGLVQPGGSLTLSCVASGSTSSINAIGWYRRAPGKQRELVAGISSDGSKVYADSVKGRFTISRDNAKNSVYLQMNSLRAEDTAVYYCYYFRTVKGSSMRYWGQGTLVTVSS

SEQ ID NO: 451 - protein, 119 aa, synthetic construct
EVQLVESGGGLVQPGGSLTLSCVASGLTSSINAMGWYRRAPGKQRELVAGISSDGSKVYQDSVKGRFTISRDNAKNSVYLQMNSLRAEDTAVYYCYYFRTNSGSSMRYWGQGTLVTVSS

SEQ ID NO: 452 - protein, 119 aa, synthetic construct
EVQLVESGGGLVQPGGSLTLSCVASGSTSSINAMAWYRRAPGKQRELVAGISSDGSKVYAESVKGRFTISRDNAKNSVYLQMNSLRAEDTAVYYCYYFRTVSGASMRYWGQGTLVTVSS

SEQ ID NO: 453 - protein, 119 aa, synthetic construct
EVQLVESGGGLVQPGGSLTLSCVASGSTSSTNAMGWYRRAPGKQRELVAGISSDGSKVLADSVKGRFTISRDNAKNSVYLQMNSLRAEDTAVYYCYYFRTVNLSSMRYWGQGTLVTVSS

SEQ ID NO: 454 - protein, 119 aa, synthetic construct
EVQLVESGGGLVQPGGSLTLSCVASGSTSSINAIGWYRRAPGKQRELVAGISSDGSKYYADSVKGRFTISRDNAKNSVYLQMNSLRAEDTAVYYCYYFRTVTGSSMRYWGQGTLVTVSS

SEQ ID NO: 455 - protein, 119 aa, synthetic construct
EVQLVESGGGLVQPGGSLTLSCVASGSTSSINAIGWYRRAPGKQRELVAGISSDGSKVYAVSVKGRFTISRDNAKNSVYLQMNSLRAEDTAVYYCYYFRKVSGSSARYWGQGTLVTVSS

SEQ ID NO: 456 - protein, 119 aa, synthetic construct
EVQLVESGGGLVQPGGSLTLSCVASGSTSSINAMGWYRRAPGKQRELVAGISSDGSKVVADSVKGRFTISRDNAKNSVYLQMNSLRAEDTAVYYCYYFRTYSGSSMRYWGQGTLVTVSS

SEQ ID NO: 457 - protein, 119 aa, synthetic construct
EVQLVESGGGLVQPGGSLTLSCVASGSTSSINAMGWYRRAPGKQRELVAGISSDGSKVYADSVKGRFTISRDNAKNSVYLQMNSLRAEDTAVYYCYYFRTVSKSSMRYWGQGTLVTVSS

SEQ ID NO: 458 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFKTVSGSSMRYWGQGTLVTVSS

SEQ ID NO: 459 - protein, 119 aa, synthetic construct
EVQLVESGGGLVQPGGSLTLSCVASGSTSSINAMAWYRRAPGKQRELAAGISSDNSKVYADSVKGRFTISRDNAKNSVYLQMNSLRAEDTAVYYCYYFRTRSGSSMRYWGQGTLVTVSS

SEQ ID NO: 460 - protein, 119 aa, synthetic construct
EVQLVESGGGLVQPGGSLTLSCVASGSKSSINAMGWYRRAPGKQRELAAGISSDGSKVYAQSVKGRFTISRDNAKNSVYLQMNSLRAEDTAVYYCYYFRTSSGSSMRYWGQGTLVTVSS

SEQ ID NO: 461 - protein, 119 aa, synthetic construct
EVQLVESGGGLVQPGGSLTLSCVASGTTSSINAMGWYRRAPGKQRELVAGISSDGSKVYVDSVKGRFTISRDNAKNSVYLQMNSLRAEDTAVYYCYYFRFLSGSSMRYWGQGTLVTVSS

SEQ ID NO: 462 - protein, 119 aa, synthetic construct
EVQLVESGGGLVQPGGSLTLSCVASGSTSSINAFGWYRRAPGKQRELVAGISSDGSKVYSDSVKGRFTISRDNAKNSVYLQMNSLRAEDTAVYYCYYFRTVSGSSMRYWGQGTLVTVSS

SEQ ID NO: 463 - protein, 119 aa, synthetic construct
EVQLVESGGGLVQPGGSLTLSCVASGSTFSINAMGWYRRAPGKQRELVAGISSDGSKVLADSVKGRFTISRDNAKNSVYLQMNSLRAEDTAVYYCYYFRLVSGSSMRYWGQGTLVTVSS

SEQ ID NO: 464 - protein, 119 aa, synthetic construct
EVQLVESGGGLVQPGGSLTLSCVASGSTRSINAMGWYRRAPGKQRELVAGISSDGSKVYNDSVKGRFTISRDNAKNSVYLQMNSLRAEDTAVYYCYYFRTVSGSSMRFWGQGTLVTVSS

SEQ ID NO: 465 - protein, 119 aa, synthetic construct
EVQLVESGGGLVQPGGSLTLSCVASGSTSSINAIGWYRRAPGKQRELVAGISSDGSKVYNDSVKGRFTISRDNAKNSVYLQMNSLRAEDTAVYYCYYFRTQSGSSMRYWGQGTLVTVSS

SEQ ID NO: 466 - protein, 119 aa, synthetic construct
EVQLVESGGGLVQPGGSLTLSCVASGSTSSINAMGWYRRAPGKQRELVAGISSDGSKVYVDSVKGRFTISRDNAKNSVYLQMNSLRAEDTAVYYCYYFRTVSGSSMPYWGQGTLVTVSS

SEQ ID NO: 467 - protein, 119 aa, synthetic construct
EVQLVESGGGLVQPGGSLTLSCVASGSTSSINAMGWYRRAPGKQRELVAGISSDGSKVVADSVKGRFTISRDNAKNSVYLQMNSLRAEDTAVYYCYYFRTLSGSSMRYWGQGTLVTVSS

SEQ ID NO: 468 - protein, 119 aa, synthetic construct
EVQLVESGGGLVQPGGSLTLSCVASGTTSSINAMGWYRRAPGKQRELVAGISSDGSKVYGDSVKGRFTISRDNAKNSVYLQMNSLRAEDTAVYYCYYFRTVSGSAMRYWGQGTLVTVSS

SEQ ID NO: 469 - protein, 119 aa, synthetic construct
EVQLVESGGGLVQPGGSLTLSCVASGSTSSINAMAWYRRAPGKQRELVAGISSDGSKVYTDSVKGRFTISRDNAKNSVYLQMNSLRAEDTAVYYCYYFRTTSGSSMRYWGQGTLVTVSS

SEQ ID NO: 470 - protein, 119 aa, synthetic construct
EVQLVESGGGLVQPGGSLTLSCVASGRTSSINAMGWYRRAPGKQRELVAGISSDGSKVYNDSVKGRFTISRDNAKNSVYLQMNSLRAEDTAVYYCYYFRTVSGTSMRYWGQGTLVTVSS

SEQ ID NO: 471 - protein, 119 aa, synthetic construct
EVQLVESGGGLVQPGGSLTLSCVASGSTSSRNAMGWYRRAPGKQRELVAGISSDGSKVTADSVKGRFTISRDNAKNSVYLQMNSLRAEDTAVYYCYYFRTRSGSSMRYWGQGTLVTVSS

SEQ ID NO: 472 - protein, 119 aa, synthetic construct
EVQLVESGGGLVQPGGSLTLSCVASGSTKSINAMGWYRRAPGKQRELVAGISSDGSKVYRDSVKGRFTISRDNAKNSVYLQMNSLRAEDTAVYYCYYFRTSSGSSMRYWGQGTLVTVSS

SEQ ID NO: 473 - protein, 119 aa, synthetic construct
EVQLVESGGGLVQPGGSLTLSCVASGSTSSRNAMGWYRRAPGKQRELVAGISSNGSKVYSDSVKGRFTISRDNAKNSVYLQMNSLRAEDTAVYYCYYFRTVSGSSMSYWGQGTLVTVSS

SEQ ID NO: 474 - protein, 119 aa, synthetic construct
EVQLVESGGGLVQPGGSLTLSCVASGSTSSINAIGWYRRAPGKQRELVAGISSDGSKVYSDSVKGRFTISRDNAKNSVYLQMNSLRAEDTAVYYCYYFRPVSGSSMRYWGQGTLVTVSS

SEQ ID NO: 475 - protein, 119 aa, synthetic construct
EVQLVESGGGLVQPGGSLTLSCVASGSTSLINAMGWYRRAPGKQRELVAGISSDGSKVYADSVKGRFTISRDNAKNSVYLQMNSLRAEDTAVYYCYYFRHVSGSSMRYWGQGTLVTVSS

SEQ ID NO: 476 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTKSGSSMRYWGQGTLVTVSS

SEQ ID NO: 477 - protein, 119 aa, synthetic construct
EVQLVESGGGLVQPGGSLTLSCVASGSTSSINAMGWYRRAPGKQRELVAGISSDGSLVYADSVKGRFTISRDNAKNSVYLQMNSLRAEDTAVYYCYYFTTVSGSSMRYWGQGTLVTVSS

SEQ ID NO: 478 - protein, 119 aa, synthetic construct
EVQLVESGGGLVQPGGSLTLSCVASGTTSSINAMGWYRRAPGKQRELVAGISSDGTKVYADSVKGRFTISRDNAKNSVYLQMNSLRAEDTAVYYCYYFHTVSGSSMRYWGQGTLVTVSS

SEQ ID NO: 479 - protein, 119 aa, synthetic construct
EVQLVESGGGLVQPGGSLTLSCVASGSTSSINAFGWYRRAPGKQRELVAGISSDGSKVYADSVKGRFTISRDNAKNSVYLQMNSLRAEDTAVYYCYYFRTVRGSSMRYWGQGTLVTVSS

SEQ ID NO: 480 - protein, 119 aa, synthetic construct
EVQLVESGGGLVQPGGSLTLSCVASGSTSSRNAMGWYRRAPGKQRELVAGISSDGSKLYLDSVKGRFTISRDNAKNSVYLQMNSLRAEDTAVYYCYYFRTVLGSSMRYWGQGTLVTVSS

SEQ ID NO: 481 - protein, 119 aa, synthetic construct
EVQLVESGGGLVQPGGSLTLSCVASGNTSSINAMGWYRRAPGKQRELVAGISSDGSRVYADSVKGRFTISRDNAKNSVYLQMNSLRAEDTAVYYCYYFRTVSGSSMRSWGQGTLVTVSS

SEQ ID NO: 482 - protein, 119 aa, synthetic construct
EVQLVESGGGLVQPGGSLTLSCVASGTTSSINAMGWYRRAPGKQRELVAGISSDGSKVYNDSVKGRFTISRDNAKNSVYLQMNSLRAEDTAVYYCYYFRTVRGSSMRYWGQGTLVTVSS

SEQ ID NO: 483 - protein, 119 aa, synthetic construct
EVQLVESGGGLVQPGGSLTLSCVASGSTASINAMGWYRRAPGKQRELVAGISSDGSKVYADSVKGRFTISRDNAKNSVYLQMNSLRAEDTAVYYCYYFRYVSGSSMRYWGQGTLVTVSS

SEQ ID NO: 484 - protein, 119 aa, synthetic construct
EVQLVESGGGLVQPGGSLTLSCVASGSTSSINAVGWYRRAPGKQRELVAGISSDGSKVYVDSVKGRFTISRDNAKNSVYLQMNSLRAEDTAVYYCYYFRTVYGSSMRYWGQGTLVTVSS

SEQ ID NO: 485 - protein, 119 aa, synthetic construct
EVQLVESGGGLVQPGGSLTLSCVASGSTSSRNAMGWYRRAPGKQRELVAGISSDGSKLYADSVKGRFTISRDNAKNSVYLQMNSLRAEDTAVYYCYYFRTVLGSSMRYWGQGTLVTVSS

SEQ ID NO: 486 - protein, 119 aa, synthetic construct
EVQLVESGGGLVQPGGSLTLSCVASGSTNSINAMGWYRRAPGKQRELVAGISSDGSKVYKDSVKGRFTISRDNAKNSVYLQMNSLRAEDTAVYYCYYYRTVSGSSMRYWGQGTLVTVSS

SEQ ID NO: 487 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRSVSGSSMRYWGQGTLVTVSS

SEQ ID NO: 488 - protein, 119 aa, synthetic construct
EVQLVESGGGLVQPGGSLTLSCVASGSTSRINAMGWYRRAPGKQRELVAGISSDGSKVYQDSVKGRFTISRDNAKNSVYLQMNSLRAEDTAVYYCYYFRRVSGSSMRYWGQGTLVTVSS

SEQ ID NO: 489 - protein, 119 aa, synthetic construct
EVQLVESGGGLVQPGGSLTLSCVPSGSTSNINAMGWYRRAPGKQRELPAGISSDGTKIYADSAKVPFTITRDNAKNSVYLQMNSLRAEDTAVYYCYYFRTVSGTSMRYWGQGTLVTVSS

SEQ ID NO: 490 - protein, 119 aa, synthetic construct
EVQLVESGGGLVQPGGSLTLSCVASGSTSKINAMGWYRRAPGKQRELVAGISSDRSKVYADSVKGRFTISRDNAKNSVYLQMNSLRAEDTAVYYCYYFRTVAGSSMRYWGQGTLVTVSS

SEQ ID NO: 491 - protein, 119 aa, synthetic construct
EVQLVESGGGLVQPGGSLTLSCVASGSTSSINALGWYRRAPGKQRELVAGISSDGSLVYADSVKGRFTISRDNAKNSVYLQMNSLRAEDTAVYYCYYFRIVSGSSMRYWGQGTLVTVSS

SEQ ID NO: 492 - protein, 119 aa, synthetic construct
EVQLVESGGGLVQPGGSLTLSCVASGSTSRINAMGWYRRAPGKQRELVAGISSDGSKVYADSVKGRFTISRDNAKNSVYLQMNSLRAEDTAVYYCYYFRTASGSSMRYWGQGTLVTVSS

SEQ ID NO: 493 - protein, 119 aa, synthetic construct
EVQLVESGGGLVQPGGSLTLSCVASGSTSSINAMGWYRRAPGKQRELVAGISSDGSKVYSDSVKGRFTISRDNAKNSVYLQMNSLRAEDTAVYYCYYFRTVSGSSSRYWGQGTLVTVSS

SEQ ID NO: 494 - protein, 119 aa, synthetic construct
EVQLVESGGGLVQPGGSLTLSCVASGSTSSINAVGWYRRAPGKQRELVAGISSDGTKVYRDSVKGRFTISRDNAKNSVYLQMNSLRAEDTAVYYCYYFRTVQGSSMRYWGQGTLVTVSS

SEQ ID NO: 495 - protein, 119 aa, synthetic construct
EVQLVESGGGLVQPGGSLTLSCVASGTTSSINAMGWYRRAPGKQRELAAGISSDGSKVYNDSVKGRFTISRDNAKNSVYLQMNSLRAEDTAVYYCYYFRTVRGSSMRYWGQGTLVTVSS

SEQ ID NO: 496 - protein, 119 aa, synthetic construct
EVQLVESGGGLVQPGGSLTLSCVASGSTSSINAMGWYRRAPGKQRELVAGISSDGSKVYADSVKGRFTISRDNAKNSVYLQMNSLRAEDTAVYYCYYFRTKSGSSMRYWGQGTLVTVSS

SEQ ID NO: 497 - protein, 119 aa, synthetic construct
EVQLVESGGGLVQPGGSLTLSCVASGTTSSINAMGWYRRAPGKQRELVAGISSDGSKVYADSVKGRFTISRDNAKNSVYLQMNSLRAEDTAVYYCYYFRTVWGSSMRYWGQGTLVTVSS

SEQ ID NO: 498 - protein, 119 aa, synthetic construct
EVQLVESGGGLVQPGGSLTLSCVASGKTSSINAMGWYRRAPGKQRELVAGISSDGSKVYTDSVKGRFTISRDNAKNSVYLQMNSLRAEDTAVYYCYYFRTRSGSSMRYWGQGTLVTVSS

SEQ ID NO: 499 - protein, 119 aa, synthetic construct
EVQLVESGGGLVQPGGSLTLSCVASGSTSSINAMGWYRRAPFKQGELPAGISPDGTKAYADSAKVRFTISRDNAKNSVYLQMNSLRAEDTAVYYCYYFHTVCGTSMGYWGQGTLVTVSS

SEQ ID NO: 500 - protein, 119 aa, synthetic construct
EVQLVESGGGLVQPGGSLTLSCVASGSTSAINAMGWYRRAPGKQRELVAGISSDGSKVYVDSVKGRFTISRDNAKNSVYLQMNSLRAEDTAVYYCYYFRTVSGSSQRYWGQGTLVTVSS

SEQ ID NO: 501 - protein, 119 aa, synthetic construct
EVQLVESGGGLVQPGGSLTLSCVASGSPSSINAYGWYRRAPGKQRELVAGISSDGSKVYSDSVKGRFTISRDNAKNSVYLQMNSLRAEDTAVYYCYYFRTVSGSSMSYWGQGTLVTVSS

SEQ ID NO: 502 - protein, 119 aa, synthetic construct
EVQLVESGGGLVQPGGSLTLSCVASGSTSSINAMGWYRRAPGKQRELVAGISSDGSKVYASSVKGRFTISRDNAKNSVYLQMNSLRAEDTAVYYCYYFRTVRGSSMRYWGQGTLVTVSS

SEQ ID NO: 503 - protein, 119 aa, synthetic construct
EVQLVESGGGLVQPGGSLTLSCVASGSRSSINAMGWYRRAPGKQRELVAGISADGSKVYADSVKGRFTISRDNAKNSVYLQMNSLRAEDTAVYYCYYFRTQSGSSMRYWGQGTLVTVSS

SEQ ID NO: 504 - protein, 119 aa, synthetic construct
EVQLVESGGGLVQPGGSLTLSCVASGSVSSINAMGWYRRAPGKQRELVAGISSDGSKVYASSAKGRFTISRDNAKNSVYLQMNSLRAEDTAVYYCYYFRTLSGSSMRYWGQGTLVTVSS

SEQ ID NO: 505 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFHTVSGSSMRYWGQGTLVTVSS

SEQ ID NO: 506 - protein, 119 aa, synthetic construct
EVQLVESGGGLVQPGGSLTLSCVASGSTSSINAIGWYRRAPGKQRELVAGISSDGSSVYADSVKGRFTISRDNAKNSVYLQMNSLRAEDTAVYYCYYFRRVSGSSMRYWGQGTLVTVSS

SEQ ID NO: 507 - protein, 119 aa, synthetic construct
EVQLVESGGGLVQPGGSLTLSCVASGSTSSINAMGWYRRAPGKQRELVAGISSDGSKVYSDSVKGRFTISRDNAKNSVYLQMNSLRAEDTAVYYCYYFRLVSGSSMRYWGQGTLVTVSS

SEQ ID NO: 508 - protein, 119 aa, synthetic construct
EVQLVESGGGLVQPGGSLTLSCVASGSTSSINAVGWYRRAPGKQRELVAGISSDGSKVYAGSVKGRFTISRDNAKNSVYLQMNSLRAEDTAVYYCYYFRTVSGSYMRYWGQGTLVTVSS

SEQ ID NO: 509 - protein, 119 aa, synthetic construct
EVQLVESGGGLVQPGGSLTLSCVASGSTSSINAMAWYRRAPGKQRELAAGISSDNSKVYADSVKGRFTISRDNAKNSVYLQMNSLRAEDTAVYYCYYFRTVGGSSMRYWGQGTLVTVSS

SEQ ID NO: 510 - protein, 119 aa, synthetic construct
EVQLVESGGGLVQPGGSLTLSCVASGSTSSINAYGWYRRAPGKQRELVAGISSDGSAVYADSVKGRFTISRDNAKNSVYLQMNSLRAEDTAVYYCYYFRTHSGSSMRYWGQGTLVTVSS

SEQ ID NO: 511 - protein, 119 aa, synthetic construct
EVQLVESGGGLVQPGGSLTLSCVASGSTSSINAVGWYRRAPGKQRELVAGISSDGSSVYADSVKGRFTISRDNAKNSVYLQMNSLRAEDTAVYYCYYFRTVSTSSMRYWGQGTLVTVSS

SEQ ID NO: 512 - protein, 119 aa, synthetic construct
EVQLVESGGGLVQPGGSLTLSCVASGSKSSINAMGWYRRAPGKQRELPAGISSNGTKVYADSAKVRFTISRDNAKNSVYLQMNSLRAEDTAVYYCYYFRTVLGTSMRYWGQGTLVTVSS

SEQ ID NO: 513 - protein, 119 aa, synthetic construct
EVQLVESGGGLVQPGGSLTLSCVASGSTSSINAMAWYRRAPGKQRELVAGISSDGSKLYADSVKGRFTISRDNAKNSVYLQMNSLRAEDTAVYYCYYFRTVSGSSMRYWGQGTLVTVSS

SEQ ID NO: 514 - protein, 119 aa, synthetic construct
EVQLVESGGGLVQPGGSLTLSCVASGSVSSINAMGWYRRAPGKQRELVAGISSDGSKVYKDSVKGRFTISRDNAKNSVYLQMNSLRAEDTAVYYCYYFRTVSGSSMGYWGQGTLVTVSS

SEQ ID NO: 515 - protein, 119 aa, synthetic construct
EVQLVESGGGLVQPGGSLTLSCVASGSTSSINAMGWYRRAPGKQRELVAGISSDGSLVYADSVKGRFTISRDNAKNSVYLQMNSLRAEDTAVYYCYYFRTVSGSSMRAWGQGTLVTVSS

SEQ ID NO: 516 - protein, 119 aa, synthetic construct
EVQLVESGGGLVQPGGSLTLSCVASGSTSSINAMGWYRRAPGKQRELVAGISSDGSLVYADSVKGRFTISRDNAKNSVYLQMNSLRAEDTAVYYCYYFRILSGSSMRYWGQGTLVTVSS

SEQ ID NO: 517 - protein, 119 aa, synthetic construct
EVQLVESGGGLVQPGGSLTLSCVASGSTSSINAMAWYRRAPGKQRELVAGISSDGSKVYADSVKGRFTISRDNAKNSVYLQMNSLRAEDTAVYYCYYFRTVQGSSMRYWGQGTLVTVSS

SEQ ID NO: 518 - protein, 119 aa, synthetic construct
EVQLVESGGGLVQPGGSLTLSCVASGSTSYINAMGWYRRAPGKQRELVAGISSDGSKVYADSVKGRFTISRDNAKNSVYLQMNSLRAEDTAVYYCYYFRTVSGQSMGYWGQGTLVTVSS

SEQ ID NO: 519 - protein, 119 aa, synthetic construct
EVQLVESGGGLVQPGGSLTLSCVASGSTSSINAVGWYRRAPGKQRELVAGISSDGSTVYADSVKGRFTISRDNAKNSVYLQMNSLRAEDTAVYYCYYFRTVSGHSMRYWGQGTLVTVSS

SEQ ID NO: 520 - protein, 119 aa, synthetic construct
EVQLVESGGGLVQPGGSLTLSCVASGSTSSINAMGWYRRAPGKQRELAAGISKDGSKVYADSAKGRFTISRDNAKNSVYLQMNSLRAEDTAVYYCYYFRTVSGSSSRYWGQGTLVTVSS

SEQ ID NO: 521 - protein, 119 aa, synthetic construct
EVQLVESGGGLVQPGGSLTLSCVASGSPSSINAYGWYRRAPGKQRELVAGISSDGSKVYSDSVKGRFTISRDNAKNSVYLQMNSLRAEDTAVYYCYYFRTVSGSSYSYWGQGTLVTVSS

SEQ ID NO: 522 - protein, 119 aa, synthetic construct
EVQLVESGGGLVQPGGSLTLSCVASGSPSSINAYGWYRRAPGKQRELVAGISSDGSKVYSDSVKGRFTISRDNAKNSVYLQMNSLRAEDTAVYYCYYFRTVSGSSQSYWGQGTLVTVSS

SEQ ID NO: 523 - protein, 117 aa, synthetic construct
EVQLVESGGGLVQPGGSLTLSCAASGSSVKFLSMAWYRQAPGKKRELVAGISADGSTDYIDSVKGRFTISRDNAKNSVYLQMNSLRAEDTAVYYCYAYRWTRRYTYWGQGTLVTVSS

SEQ ID NO: 524 - protein, 117 aa, synthetic construct
EVQLVESGGGLVQPGGSLTLSCAASGSSVSFLSLAWYRQAPGKKRELVAGISADGSTAYIDSVKGRFTISRDNAKNSVYLQMNSLRAEDTAVYYCYAYRWRTRYTYWGQGTLVTVSS

SEQ ID NO: 525 - protein, 117 aa, synthetic construct
EVQLVESGGGLVQPGGSLTLSCAASGSRVSFLSMAWYRQAPGKKRELVAGISRDGSTDYIDSVKGRFTISRDNAKNSVYLQMNSLRAEDTAVYYCYAYRWTTRRTYWGQGTLVTVSS

SEQ ID NO: 526 - protein, 117 aa, synthetic construct
EVQLVESGGGLVQPGGSLTLSCAASGSQVSFLSMAWYRQAPGKKRELVAGISRDGSTDYIDSVKGRFTISRDNAKNSVYLQMNSLRAEDTAVYYCYAYRWTTRYIYWGQGTLVTVSS

SEQ ID NO: 527 - protein, 117 aa, synthetic construct
EVQLVESGGGLVQPGGSLTLSCAASGSSVSFLSMAWYRQAPGKKRELVAGISEAGSTDYIDSVKGRFTISRDNAKNSVYLQMNSLRAEDTAVYYCYAYRWRTRYTYWGQGTLVTVSS

SEQ ID NO: 528 - protein, 117 aa, synthetic construct
EVQLVESGGGLVQPGGSLTLRCAASGSKVSFLSMAWYRQAPGKKRELVAGISADGSTDYVDSVKGRFTISRDNAKNSVYLQMNSLRAEDTAVYYCYAYRWTRRYTYWGQGTLVTVSS

SEQ ID NO: 529 - protein, 117 aa, synthetic construct
EVQLVESGGGLVQPGGSLTLSCAASGSSVGFLSMAWYRQAPGKKRELVAGISADGSTDYIRSVKGRFTISRDNAKNSVYLQMNSLRAEDTAVYYCYAYRWRTRYTYWGQGTLVTVSS

SEQ ID NO: 530 - protein, 117 aa, synthetic construct
EVQLVESGGGLVQPGGSLTLSCAASGSSVSFLSMAWYRQAPGKKRELVAGISADGSVDYIDSVKGRFTISRDNAKNSVYLQMNSLRAEDTAVYYCYAYRWTTRYIYWGQGTLVTVSS

SEQ ID NO: 531 - protein, 117 aa, synthetic construct
EVQLVESGGGLVQPGGSLTLSCAASGSRVSFLSMAWYRQAPGKKRELVAGISADGSTLYIDSVKGRFTISRDNAKNSVYLQMNSLRAEDTAVYYCYAYRWTTRRTYWGQGTLVTVSS

SEQ ID NO: 532 - protein, 117 aa, synthetic construct
EVQLVESGGGLVQPGGSLTLSCAASGSSVSFLSLAWYRQAPGKKRELVAGISTDGSTDYIDSVKGRFTISRDNAKNSVYLQMNSLRAEDTAVYYCYAYRWRTRYTYWGQGTLVTVSS

SEQ ID NO: 533 - protein, 117 aa, synthetic construct
EVQLVESGGGLVQPGGSLTLSCAASGSSVSFLSMAWYRQAPGKKRELVAGISGDGSTDYIDSVKGRFTISRDNAKNSVYLQMNSLRAEDTAVYYCYAYRWTTRYTYWGQGTLVTVSS

SEQ ID NO: 534 - protein, 117 aa, synthetic construct
EVQLVESGGGLVQPGGSLTLSCAASGSSVQFLSMAWYRQAPGKKRELVAGISADGSTDYINSVKGRFTISRDNAKNSVYLQMNSLRAEDTAVYYCYAYRWRTRYTYWGQGTLVTVSS

SEQ ID NO: 535 - protein, 117 aa, synthetic construct
EVQLVESGGGLVQPGGSLTLSCAASGSNVSFLSMAWYRQAPGKKRELVAGISARGSTDYIDSVKGRFTISRDNAKNSVYLQMNSLRAEDTAVYYCYAYHWTTRYTYWGQGTLVTVSS

SEQ ID NO: 536 - protein, 117 aa, synthetic construct
EVQLVESGGGLVQPGGSLTLSCVASGSSVKFLSMAWYRQAPGKKRELVAGISADGSTTYIDSVKGRFTISRDNAKNSVYLQMNSLRAEDTAVYYCYAYRWTTRRTYWGQGTLVTVSS

SEQ ID NO: 537 - protein, 117 aa, synthetic construct
EVQLVESGGGLVQPGGSLTLSCAASGKSVSFLSMAWYRQAPGKKRELVAGISKDGSTDYIDSVKGRFTISRDNAKNSVYLQMNSLRAEDTAVYYCYAYRWTTRYTYWGQGTLVTVSS

SEQ ID NO: 538 - protein, 117 aa, synthetic construct
EVQLVESGGGLVQPGGSLTLSCAASGSRVSFLSMAWYRQAPGKKRELVAGISADGSTTYIDSVKGRFTISRDNAKNSVYLQMNSLRAEDTAVYYCYAYRWTTRYTYWGQGTLVTVSS

SEQ ID NO: 539 - protein, 117 aa, synthetic construct
EVQLVESGGGLVQPGGSLTLSCAASGSHVSFLSMAWYRQAPGKKRELVAGISANGSTDYIDSVKGRFTISRDNAKNSVYLQMNSLRAEDTAVYYCYAYRWTTRYAYWGQGTLVTVSS

SEQ ID NO: 540 - protein, 117 aa, synthetic construct
EVQLVESGGGLVQPGGSLTLSCAASGSSVSFLSMAWYRQAPGKKRELVAGISRDGSTDYIDSVKGRFTISRDNAKNSVYLQMNSLRAEDTAVYYCYAYRWVTRYTYWGQGTLVTVSS

SEQ ID NO: 541 - protein, 117 aa, synthetic construct
EVQLVESGGGLVQPGGSLTLSCAASGSSVSFLSMAWYRQAPGKKRELVAGISADGSADYIDSVKGRFTISRDNAKNSVYLQMNSLRAEDTAVYYCYAYRWVTRYTYWGQGTLVTVSS

SEQ ID NO: 542 - protein, 117 aa, synthetic construct
EVQLVESGGGLVQPGGSLTLSCAASGSSVRFLSMAWYRQAPGKKRELVAGISAHGSTDYIDSVKGRFTISRDNAKNSVYLQMNSLRAEDTAVYYCYAYRWSTRYTYWGQGTLVTVSS

SEQ ID NO: 543 - protein, 117 aa, synthetic construct
EVQLVESGGGLVQPGGSLTLSCAASGSSVRFLSMAWYRQAPGKKRELVAGISADGSTIYIDSVKGRFTISRDNAKNSVYLQMNSLRAEDTAVYYCYAYRWRTRYTYWGQGTLVTVSS

SEQ ID NO: 544 - protein, 117 aa, synthetic construct
EVQLVESGGGLVQPGGSLTLSCAASGSSVRFLSMAWYRQAPGKKRELVAGISRDGSTVYIDSVKGRFTISRDNAKNSVYLQMNSLRAEDTAVYYCYAYRWTTRGTYWGQGTLVTVSS

SEQ ID NO: 545 - protein, 117 aa, synthetic construct
EVQLVESGGGLVQPGGSLTLSCAASGSHVSFLSMAWYRQAPGKKRELVAGISADGPTDYIDSVKGRFTISRDNAKNSVYLQMNSLRAEDTAVYYCYAYRWDTRYTYWGQGTLVTVSS

SEQ ID NO: 546 - protein, 117 aa, synthetic construct
EVQLVESGGGLVQPGGSLTLSCVASGTSVSFLSMAWYRQAPGKKRELVAGISADGSTTYIDSVKGRFTISRDNAKNSVYLQMNSLRAEDTAVYYCYAYRWTTRRTYWGQGTLVTVSS

SEQ ID NO: 547 - protein, 117 aa, synthetic construct
EVQLVESGGGLVQPGGSLTLSCAASGTSVSFLSIAWYRQAPGKKRELVAGISADGSTDYIASVKGRFTISRDNAKNSVYLQMNSLRAEDTAVYYCYAYRWRTRYTYWGQGTLVTVSS

SEQ ID NO: 548 - protein, 117 aa, synthetic construct
EVQLVESGGGLVQPGGSLTLSCAASGSSVKFLSMAWYRQAPGKKRELVAGISLDGSTDYIDSVKGRFTISRDNAKNSVYLQMNSLRAEDTAVYYCYAYRWTGRYTYWGQGTLVTVSS

SEQ ID NO: 549 - protein, 117 aa, synthetic construct
EVQLVESGGGLVQPGGSLTLSCAASGSSVSFLSMAWYRQAPGKKRELVAGISADGSTIYIDSVKGRFTISRDNAKNSVYLQMNSLRAEDTAVYYCYAYRWRTRYTYWGQGTLVTVSS

SEQ ID NO: 550 - protein, 117 aa, synthetic construct
EVQLVESGGGLVQPGGSLTLSCAASGSSVRFLSMAWYRQAPGKKRELVAGISAHGSTDYIDSVKGRFTISRDNAKNSVYLQMNSLRAEDTAVYYCYAYRWRTRYTYWGQGTLVTVSS

SEQ ID NO: 551 - protein, 117 aa, synthetic construct
EVQLVESGGGLVQPGGSLTLSCAASGSSVSFLSMAWYRQAPGKKRELVAGISRDGSTDYIDSVKGRFTISRDNAKNSVYLQMNSLRAEDTAVYYCYAYRWITRYTYWGQGTLVTVSS

SEQ ID NO: 552 - protein, 117 aa, synthetic construct
EVQLVESGGGLVQPGGSLTLSCAASGSSVRFLSMAWYRQAPGKKRELVAGISRDGSTDYIDSVKGRFTISRDNAKNSVYLQMNSLRAEDTAVYYCYAYRWITRYTYWGQGTLVTVSS

SEQ ID NO: 553 - protein, 117 aa, synthetic construct
EVQLVESGGGLVQPGGSLTLSCAASGSSVVFLSMAWYRQAPGKKRELVAGISADGSMDYIDSVKGRFTISRDNAKNSVYLQMNSLRAEDTAVYYCYAYRWRTRYTYWGQGTLVTVSS

SEQ ID NO: 554 - protein, 117 aa, synthetic construct
EVQLVESGGGLVQPGGSLTLSCAASGSSVRFLSMAWYRQAPGKKRELVAGISADGSTDYIDSVKGRFTISRDNAKNSVYLQMNSLRAEDTAVYYCYAYRWTRRYTYWGQGTLVTVSS

SEQ ID NO: 555 - protein, 117 aa, synthetic construct
EVQLVESGGGLVQPGGSLTLSCAASGSSVRFLSMAWYRQAPGKKRELVAGISADGSTDYIDSVKGRFTISRDNAKNSVYLQMNSLRAEDTAVYYCYAYSWTTRYTYWGQGTLVTVSS

SEQ ID NO: 556 - protein, 117 aa, synthetic construct
EVQLVESGGGLVQPGGSLTLSCAASGSSVRFLSMAWYRQAPGKKRELVAGISANGSTDYIDSVKGRFTISRDNAKNSVYLQMNSLRAEDTAVYYCYAYRWTNRYTYWGQGTLVTVSS

SEQ ID NO: 557 - protein, 117 aa, synthetic construct
EVQLVESGGGLVQPGGSLTLSCAASGSSVSRLSMAWYRQAPGKKRELVAGISANGSTTYIDSVKGRFTISRDNAKNSVYLQMNSLRAEDTAVYYCYAYRWTTRYRYWGQGTLVTVSS

SEQ ID NO: 558 - protein, 117 aa, synthetic construct
EVQLVESGGGLVQPGGSLTLSCAASGSSKSFLSMAWYRQAPGKKRELVAGISADGSTSYIDSVKGRFTISRDNAKNSVYLQMNSLRAEDTAVYYCYAYRWTTRYTYWGQGTLVTVSS

SEQ ID NO: 559 - protein, 117 aa, synthetic construct
EVQLVESGGGLVQPGGSLTLSCAASGSSVSRLSMAWYRQAPGKKRELVAGISADGSRDYIDSVKGRFTISRDNAKNSVYLQMNSLRAEDTAVYYCYAYRWTTRYKYWGQGTLVTVSS

SEQ ID NO: 560 - protein, 117 aa, synthetic construct
EVQLVESGGGLVQPGGSLTLSCAASGSSVKFLSMAWYRQAPGKKRELVAGISADGSTMYIDSVKGRFTISRDNAKNSVYLQMNSLRAEDTAVYYCYAYRWHTRYTYWGQGTLVTVSS

SEQ ID NO: 561 - protein, 117 aa, synthetic construct
EVQLVESGGGLVQPGGSLTLSCAASGSGVRFLSMAWYRQAPGKKRELVAGISPDGSTDYIDSVKGRFTISRDNAKNSVYLQMNSLRAEDTAVYYCYAYRWTRRYTYWGQGTLVTVSS

SEQ ID NO: 562 - protein, 117 aa, synthetic construct
EVQLVESGGGLVQPGGSLTLSCAASGSSVRFLSMAWYRQAPGKKRELVAGISGDGSTDYIDSVKGRFTISRDNAKNSVYLQMNSLRAEDTAVYYCYAYRWMTRYTYWGQGTLVTVSS

SEQ ID NO: 563 - protein, 117 aa, synthetic construct
EVQLVESGGGLVQPGGSLTLSCAASGSSVHFLSMAWYRQAPGKKRELVAGISRDGSTDYIDSVKGRFTISRDNAKNSVYLQMNSLRAEDTAVYYCYAYRWTTRYRYWGQGTLVTVSS

SEQ ID NO: 564 - protein, 117 aa, synthetic construct
EVQLVESGGGLVQPGGSLTLSCAASGSSVRFLSMAWYRQAPGKKRELVAGISRDGSTDYIDSVKGRFTISRDNAKNSVYLQMNSLRAEDTAVYYCYAYRWSTRYTYWGQGTLVTVSS

SEQ ID NO: 565 - protein, 117 aa, synthetic construct
EVQLVESGGGLVQPGGSLTLSCAASGSKVSFLSMAWYRQAPGKKRELVAGISRDGSTDYIDSVKGRFTISRDNAKNSVYLQMNSLRAEDTAVYYCYAYRWTTRYTFWGQGTLVTVSS

SEQ ID NO: 566 - protein, 117 aa, synthetic construct
EVQLVESGGGLVQPGGSLTLSCAASGSSVSFLSMAWYRQAPGKKRELVAGISADGSTDYIDSVKGRFTISRDNAKNSVYLQMNSLRAEDTAVYYCYAYRWRTRYTYWGQGTLVTVSS

SEQ ID NO: 567 - protein, 117 aa, synthetic construct
EVQLVESGGGLVQPGGSLTLSCAASGSKVSFLSMAWYRQAPGKKRELVAGISTDGSTDYIDSVKGRFTISRDNAKNSVYLQMNSLRAEDTAVYYCYAYRWTTRRTYWGQGTLVTVSS

SEQ ID NO: 568 - protein, 117 aa, synthetic construct
EVQLVESGGGLVQPGGSLTLSCAASGSRVSFLSMAWYRQAPGKKRELVAGISADGSTSYIDSVKGRFTISRDNAKNSVYLQMNSLRAEDTAVYYCYAYRWATRYTYWGQGTLVTVSS

SEQ ID NO: 569 - protein, 117 aa, synthetic construct
EVQLVESGGGLVQPGGSLTLSCAASGSRVSFLSMAWYRQAPGKKRELVAGISADGSTLYIDSVKGRFTISRDNAKNSVYLQMNSLRAEDTAVYYCYAYRWHTRYTYWGQGTLVTVSS

SEQ ID NO: 570 - protein, 117 aa, synthetic construct
EVQLVESGGGLVQPGGSLTLSCAASGSSVRFLSMAWYRQAPGKKRELVAGISRDGSTDYIDSVKGRFTISRDNAKNSVYLQMNSLRAEDTAVYYCYAYRWGTRYTYWGQGTLVTVSS

SEQ ID NO: 571 - protein, 117 aa, synthetic construct
EVQLVESGGGLVQPGGSLTLSCAASYSSVSRLSMAWYRQAPGKKRELVAGISADGSTVYIDSVKGRFTISRDNAKNSVYLQMNSLRAEDTAVYYCYAYRWTTRNTYWGQGTLVTVSS

SEQ ID NO: 572 - protein, 117 aa, synthetic construct
EVQLVESGGGLVQPGGSLTLSCAASGSSVSFLSMAWYRQAPGKKRELVAGISTDGSTDYIDSVKGRFTISRDNAKNSVYLQMNSLRAEDTAVYYCYAYRWRTRYTYWGQGTLVTVSS

SEQ ID NO: 573 - protein, 117 aa, synthetic construct
EVQLVESGGGLVQPGGSLTLSCAASGSSVSFLSMAWYRQAPGKKRELVAGISADGSTLYIDSVKGRFTISRDNAKNSVYLQMNSLRAEDTAVYYCYAYRWTTRYAYWGQGTLVTVSS

SEQ ID NO: 574 - protein, 117 aa, synthetic construct
EVQLVESGGGLVQPGGSLTLSCAASGSSVSFLSMAWYRQAPGKKRELVAGISADGRTDYIDSVKGRFTISRDNAKNSVYLQMNSLRAEDTAVYYCYAYRWRTRYTYWGQGTLVTVSS

SEQ ID NO: 575 - protein, 117 aa, synthetic construct
EVQLVESGGGLVQPGGSLTLSCVASGTSVSFLSMAWYRQAPGKKRELVAGISADGSTIYIDSVKGRFTISRDNAKNSVYLQMNSLRAEDTAVYYCYAYRWTTRRTYWGQGTLVTVSS

SEQ ID NO: 576 - protein, 117 aa, synthetic construct
EVQLVESGGGLVQPGGSLTLSCAASGSSVKFLSMAWYRQAPGKKRELVAGISADGSTLYIDSVKGRFTISRDNAKNSVYLQMNSLRAEDTAVYYCYAYRWTTRRTYWGQGTLVTVSS

SEQ ID NO: 577 - protein, 117 aa, synthetic construct
EVQLVESGGGLVQPGGSLTLSCAASGSSVSFLSMAWYRQAPGKKRELVAGISRDGSTDYIDSVKGRFTISRDNAKNSVYLQMNSLRAEDTAVYYCYAYRWTSRYTYWGQGTLVTVSS

SEQ ID NO: 578 - protein, 117 aa, synthetic construct
EVQLVESGGGLVQPGGSLTLSCAASGSRVSFLSMAWYRQAPGKKRELVAGISKDGSTDYIDSVKGRFTISRDNAKNSVYLQMNSLRAEDTAVYYCYAYRWTTRVTYWGQGTLVTVSS

SEQ ID NO: 579 - protein, 117 aa, synthetic construct
EVQLVESGGGLVQPGGSLTLSCAASGSSVSVLSMAWYRQAPGKKRELVAGISADGSTDYIGSVKGRFTISRDNAKNSVYLQMNSLRAEDTAVYYCYAYRWTTRTTYWGQGTLVTVSS

SEQ ID NO: 580 - protein, 117 aa, synthetic construct
EVQLVESGGGLVQPGGSLTLSCAASGSSVSFLSMAWYRQAPGKKRELVAGISVDGSTDYIDSVKGRFTISRDNAKNSVYLQMNSLRAEDTAVYYCYAYRWRTRYTYWGQGTLVTVSS

SEQ ID NO: 581 - protein, 117 aa, synthetic construct
EVQLVESGGGLVQPGGSLTLSCAASGSRVSFLSMAWYRQAPGKKRELVAGISADGSTGYIDSVKGRFTISRDNAKNSVYLQMNSLRAEDTAVYYCYAYRWATRYTYWGQGTLVTVSS

SEQ ID NO: 582 - protein, 117 aa, synthetic construct
EVQLVESGGGLVQPGGSLTLSCVASGSSVKFLSMAWYRQAPGKKRELVAGISGDGSTTYIDSVKGRFTISRDNAKNSVYLQMNSLRAEDTAVYYCYAYRWTTRRTYWGQGTLVTVSS

SEQ ID NO: 583 - protein, 117 aa, synthetic construct
EVQLVESGGGLVQPGGSLTLSCAASGSSVRFLSMAWYRQAPGKKRELVAGISTDGSTDYIDSVKGRFTISRDNAKNSVYLQMNSLRAEDTAVYYCYALRWTTRYTYWGQGTLVTVSS

SEQ ID NO: 584 - protein, 117 aa, synthetic construct
EVQLVESGGGLVQPGGSLTLSCAASGSSVSQLSMAWYRQAPGKKRELVAGISADGSTDYFDSVKGRFTISRDNAKNSVYLQMNSLRAEDTAVYYCYAYRWTTRGTYWGQGTLVTVSS

SEQ ID NO: 585 - protein, 117 aa, synthetic construct
EVQLVESGGGLVQPGGSLTLSCAASGSRVSFLSMAWYRQAPGKKRELVAGISADGSTSYIDSVKGRFTISRDNAKNSVYLQMNSLRAEDTAVYYCYAYRWRTRYTYWGQGTLVTVSS

SEQ ID NO: 586 - protein, 117 aa, synthetic construct
EVQLVESGGGLVQPGGSLTLSCAASKSSVSFLSMAWYRQAPGKKRELVAGISADGSTDYIDSVKGRFTISRDNAKNSVYLQMNSLRAEDTAVYYCYAYRWTTRATYWGQGTLVTVSS

SEQ ID NO: 587 - protein, 117 aa, synthetic construct
EVQLVESGGGLVQPGGSLTLSCAASGSSVSFLSMAWYRQAPGKKRELVAGISADGSTAYIDSVKGRFTISRDNAKNSVYLQMNSLRAEDTAVYYCYAYRWTTRRTYWGQGTLVTVSS

SEQ ID NO: 588 - protein, 117 aa, synthetic construct
EVQLVESGGGLVQPGGSLTLSCAASGSSVKFLSMAWYRQAPGKKRELVAGISADGSTVYIDSVKGRFTISRDNAKNSVYLQMNSLRAEDTAVYYCYAYRWPTRYTYWGQGTLVTVSS

SEQ ID NO: 589 - protein, 117 aa, synthetic construct
EVQLVESGGGLVQPGGSLTLSCAASGSSVRFLSMAWYRQAPGKKRELVAGISQDGSTDYIDSVKGRFTISRDNAKNSVYLQMNSLRAEDTAVYYCYAYRWRTRYTYWGQGTLVTVSS

SEQ ID NO: 590 - protein, 117 aa, synthetic construct
EVQLVESGGGLVQPGGSLTLSCAASGSSVRFLSMAWYRQAPGKKRELVAGISNDGSTDYIDSVKGRFTISRDNAKNSVYLQMNSLRAEDTAVYYCYAYRWKTRYTYWGQGTLVTVSS

SEQ ID NO: 591 - protein, 117 aa, synthetic construct
EVQLVESGGGLVQPGGSLTLSCAASGSRVSFLSMAWYRQAPGKKRELVAGISARGSTDYIDSVKGRFTISRDNAKNSVYLQMNSLRAEDTAVYYCYAYRWSTRYTYWGQGTLVTVSS

SEQ ID NO: 592 - protein, 117 aa, synthetic construct
EVQLVESGGGLVQPGGSLTLSCAASGSSVSFLSLAWYRQAPGKKRELVAGISADGSTDYIDSVKGRFTISRDNAKNSVYLQMNSLRAEDTAVYYCYAYRWKTRRTYWGQGTLVTVSS

SEQ ID NO: 593 - protein, 117 aa, synthetic construct
EVQLVESGGGLVQPGGSLTLSCAASGSSVRFLSMAWYRQAPGKKRELVAGISRDGSTDYIDSVKGRFTISRDNAKNSVYLQMNSLRAEDTAVYYCYAYRWTTRRTYWGQGTLVTVSS

SEQ ID NO: 594 - protein, 117 aa, synthetic construct
EVQLVESGGGLVQPGGSLTLSCAASGSKVSFLSMAWYRQAPGKKRELVAGISADGSTLYIDSVKGRFTISRDNAKNSVYLQMNSLRAEDTAVYYCYAYRWTTRYRYWGQGTLVTVSS

SEQ ID NO: 595 - protein, 117 aa, synthetic construct
EVQLVESGGGLVQPGGSLTLSCAASGSSVSFLSMAWYRQAPGKKRELVAGISADGSTNYIDSVKGRFTISRDNAKNSVYLQMNSLRAEDTAVYYCYAYRWRTRYTYWGQGTLVTVSS

SEQ ID NO: 596 - protein, 117 aa, synthetic construct
EVQLVESGGGLVQPGGSLTLSCAASGSSVRFLSMAWYRQAPGKKRELVAGISADGSTVYIDSVKGRFTISRDNAKNSVYLQMNSLRAEDTAVYYCYAYRWTTRYKYWGQGTLVTVSS

SEQ ID NO: 597 - protein, 117 aa, synthetic construct
EVQLVESGGGLVQPGGSLTLSCAASGSKVSFLSMAWYRQAPGKKRELVAGISADGSTTYIDSVKGRFTISRDNAKNSVYLQMNSLRAEDTAVYYCYAYRWKTRYTYWGQGTLVTVSS

SEQ ID NO: 598 - protein, 117 aa, synthetic construct
EVQLVESGGGLVQPGGSLTLSCAASGSSVKFLSMAWYRQAPGKKRELVAGISADGSTDYIGSVKGRFTISRDNAKNSVYLQMNSLRAEDTAVYYCYAYRWTTRVTYWGQGTLVTVSS

SEQ ID NO: 599 - protein, 117 aa, synthetic construct
EVQLVESGGGLVQPGGSLTLSCAASGSSVKFLSMAWYRQAPGKKRELVAGISRDGSTDYIDSVKGRFTISRDNAKNSVYLQMNSLRAEDTAVYYCYAYRWTTRFTYWGQGTLVTVSS

SEQ ID NO: 600 - protein, 117 aa, synthetic construct
EVQLVESGGGLVQPGGSLTLSCAASGSRVSFLSMAWYRQAPGKKRELVAGISADGSTTYIDSVKGRFTISRDNAKNSVYLQMNSLRAEDTAVYYCYAYRWTTRFTYWGQGTLVTVSS

SEQ ID NO: 601 - protein, 117 aa, synthetic construct
EVQLVESGGGLVQPGGSLTLSCAASGSSVLFLSMAWYRQAPGKKRELVAGVSSDGSTDYIDSVKGRFTISRDNAKNSVYLQMNSLRAEDTAVYYCYAYRWRTRYTYWGQGTLVTVSS

SEQ ID NO: 602 - protein, 117 aa, synthetic construct
EVQLVESGGGLVQPGGSLTLSCAASGSRVSFLSMAWYRQAPGKKRELVAGISADGHTDYIDSVKGRFTISRDNAKNSVYLQMNSLRAEDTAVYYCYAYRWTTRYTHWGQGTLVTVSS

SEQ ID NO: 603 - protein, 117 aa, synthetic construct
EVQLVESGGGLVQPGGSLTLSCAASGSRVSFLSMAWYRQAPGKKRELVAGISADGSTDYFDSVKGRFTISRDNAKNSVYLQMNSLRAEDTAVYYCYAYRWTRRYTYWGQGTLVTVSS

SEQ ID NO: 604 - protein, 117 aa, synthetic construct
EVQLVESGGGLVQPGGSLTLSCAASGSSVGFLSMAWYRQAPGKKRELVAGISADGSTVYIDSVKGRFTISRDNAKNSVYLQMNSLRAEDTAVYYCYAYRWTTRYTYWGQGTLVTVSS

SEQ ID NO: 605 - protein, 117 aa, synthetic construct
EVQLVESGGGLVQPGGSLTLSCAASGSSVSFMSMAWYRQAPGKKRELVAGISADGSTDYIASVKGRFTISRDNAKNSVYLQMNSLRAEDTAVYYCYAYRWTTRSTYWGQGTLVTVSS

SEQ ID NO: 606 - protein, 117 aa, synthetic construct
EVQLVESGGGLVQPGGSLTLSCAASGSSVSFLSMAWYRQAPGKKRELVAGISADGSTDYISSVKGRFTISRDNAKNSVYLQMNSLRAEDTAVYYCYAYSWTTRYTYWGQGTLVTVSS

SEQ ID NO: 607 - protein, 117 aa, synthetic construct
EVQLVESGGGLVQPGGSLTLSCAASGSSVTFLSMAWYRQAPGKKRELVAGISADGSTVYIDSVKGRFTISRDNAKNSVYLQMNSLRAEDTAVYYCYAYRWTTRGTYWGQGTLVTVSS

SEQ ID NO: 608 - protein, 117 aa, synthetic construct
EVQLVESGGGLVQPGGSLTLSCAASGSSVRFLSMAWYRQAPGKKRELVAGISADGSTVYIDSVKGRFTISRDNAKNSVYLQMNSLRAEDTAVYYCYAYRWKTRYTYWGQGTLVTVSS

SEQ ID NO: 609 - protein, 117 aa, synthetic construct
EVQLVESGGGLVQPGGSLTLSCAASGSKVSFLSMAWYRQAPGKKRELVAGISADGSTTYIDSVKGRFTISRDNAKNSVYLQMNSLRAEDTAVYYCYAYRWTTRFTYWGQGTLVTVSS

SEQ ID NO: 610 - protein, 117 aa, synthetic construct
EVQLVESGGGLVQPGGSLTLSCAASGSKVSFMSMAWYRQAPGKKRELVAGISVDGSTDYIDSVKGRFTISRDNAKNSVYLQMNSLRAEDTAVYYCYAYRWRTRYTYWGQGTLVTVSS

SEQ ID NO: 611 - protein, 117 aa, synthetic construct
EVQLVESGGGLVQPGGSLTLSCAASGSSVSNLSMAWYRQAPGKKRELVAGISADGSTAYIDSVKGRFTISRDNAKNSVYLQMNSLRAEDTAVYYCYAYRWTTRRTYWGQGTLVTVSS

SEQ ID NO: 612 - protein, 117 aa, synthetic construct
EVQLVESGGGLVQPGGSLTLSCAASNSSVSKLSMAWYRQAPGKKRELVAGISADGSTAYIDSVKGRFTISRDNAKNSVYLQMNSLRAEDTAVYYCYAYRWSTRYTYWGQGTLVTVSS

SEQ ID NO: 613 - protein, 117 aa, synthetic construct
EVQLVESGGGLVQPGGSLTLSCAASGSKVSFLSMAWYRQAPGKKRELVAGISADGSKDYIDSVKGRFTISRDNAKNSVYLQMNSLRAEDTAVYYCYAYRWTTRLTYWGQGTLVTVSS

SEQ ID NO: 614 - protein, 117 aa, synthetic construct
EVQLVESGGGLVQPGGSLTLSCVASGSQVSFLSMAWYRQAPGKKRELVAGISADGSTDYFDSVKGRFTISRDNAKNSVYLQMNSLRAEDTAVYYCYAYRWTRRYTYWGQGTLVTVSS

SEQ ID NO: 615 - protein, 117 aa, synthetic construct
EVQLVESGGGLVQPGGSLTLSCAASGSKVSFMSMAWYRQAPGKKRELVAGISADGSTDYIDSVKGRFTISRDNAKNSVYLQMNSLRAEDTAVYYCYAYRWTTRLTYWGQGTLVTVSS

SEQ ID NO: 616 - protein, 117 aa, synthetic construct
EVQLVESGGGLVQPGGSLTLSCAASGSRVSFLSMAWYRQAPGKKRELVAGISADGSTVYIDSVKGRFTISRDNAKNSVYLQMNSLRAEDTAVYYCYAYRWTRRYTYWGQGTLVTVSS

SEQ ID NO: 617 - protein, 117 aa, synthetic construct
EVQLVESGGGLVQPGGSLTLSCAASGSSVRFLSMAWYRQAPGKKRELVAGISADGSTVYIDSVKGRFTISRDNAKNSVYLQMNSLRAEDTAVYYCYAYRWTTRRTYWGQGTLVTVSS

SEQ ID NO: 618 - protein, 117 aa, synthetic construct
EVQLVESGGGLVQPGGSLTLSCAASGSKVSFLSMAWYRQAPGKKRELVAGISARGSTDYIDSVKGRFTISRDNAKNSVYLQMNSLRAEDTAVYYCYAYQWTTRYTYWGQGTLVTVSS

SEQ ID NO: 619 - protein, 117 aa, synthetic construct
EVQLVESGGGLVQPGGSLTLSCAASGSSVRFLSMAWYRQAPGKKRELVAGISATGSTDYIDSVKGRFTISRDNAKNSVYLQMNSLRAEDTAVYYCYAYRWTRRYTYWGQGTLVTVSS

SEQ ID NO: 620 - protein, 117 aa, synthetic construct
EVQLVESGGGLVQPGGSLTLSCAASGSSVSFLSIAWYRQAPGKKRELVAGISKDGSTDYIDSVKGRFTISRDNAKNSVYLQMNSLRAEDTAVYYCYAYRWTTRMTYWGQGTLVTVSS

SEQ ID NO: 621 - protein, 117 aa, synthetic construct
EVQLVESGGGLVQPGGSLTLSCAASGSSSSFLSMAWYRQAPGKKRELVAGISADGSTVYIDSVKGRFTISRDNAKNSVYLQMNSLRAEDTAVYYCYAYRWTTRRTYWGQGTLVTVSS

SEQ ID NO: 622 - protein, 117 aa, synthetic construct
EVQLVESGGGLVQPGGSLTLSCAASGSSVKFLSMAWYRQAPGKKRELVAGISPDGSTDYIDSVKGRFTISRDNAKNSVYLQMNSLRAEDTAVYYCYAYRWTTRYRYWGQGTLVTVSS

SEQ ID NO: 623 - protein, 117 aa, synthetic construct
EVQLVESGGGLVQPGGSLTLSCAASGSSVNFLSMAWYRQAPGKKRELVAGISADGSTHYIDSVKGRFTISRDNAKNSVYLQMNSLRAEDTAVYYCYAYRWLTRYTYWGQGTLVTVSS

SEQ ID NO: 624 - protein, 117 aa, synthetic construct
EVQLVESGGGLVQPGGSLTLSCAASGSSVKFLSMAWYRQAPGKKRELVAGISADGSTDYILSVKGRFTISRDNAKNSVYLQMNSLRAEDTAVYYCYAYEWTTRYTYWGQGTLVTVSS

SEQ ID NO: 625 - protein, 117 aa, synthetic construct
EVQLVESGGGLVQPGGSLTLSCAASGSSVSFLSMAWYRQAPGKKRELVAGISADGSTDYIHSVKGRFTISRDNAKNSVYLQMNSLRAEDTAVYYCYAYRWRTRYTYWGQGTLVTVSS

SEQ ID NO: 626 - protein, 117 aa, synthetic construct
EVQLVESGGGLVQPGGSLTLSCAASGSSVRFLSMAWYRQAPGKKRELVAGISVDGSTDYIDSVKGRFTISRDNAKNSVYLQMNSLRAEDTAVYYCYAYRWRTRYTYWGQGTLVTVSS

SEQ ID NO: 627 - protein, 117 aa, synthetic construct
EVQLVESGGGLVQPGGSLTLSCAASGSSVSFLSVAWYRQAPGKKRELVAGISRDGSTDYIDSVKGRFTISRDNAKNSVYLQMNSLRAEDTAVYYCYAYRWRTRYTYWGQGTLVTVSS

SEQ ID NO: 628 - protein, 117 aa, synthetic construct
EVQLVESGGGLVQPGGSLTLSCAASGSQVSFLSMAWYRQAPGKKRELVAGISADGSTVYIDSVKGRFTISRDNAKNSVYLQMNSLRAEDTAVYYCYAYRWSTRYTYWGQGTLVTVSS

SEQ ID NO: 629 - protein, 117 aa, synthetic construct
EVQLVESGGGLVQPGGSLTLSCAASGTSVSFLSMAWYRQAPGKKRELVAGISADGSTDYIRSVKGRFTISRDNAKNSVYLQMNSLRAEDTAVYYCYAYRWTTRLTYWGQGTLVTVSS

SEQ ID NO: 630 - protein, 117 aa, synthetic construct
EVQLVESGGGLVQPGGSLTLSCAASGSRVSFLSMAWYRQAPGKKRELVAGISADGSTMYIDSVKGRFTISRDNAKNSVYLQMNSLRAEDTAVYYCYAYRWTTRLTYWGQGTLVTVSS

SEQ ID NO: 631 - protein, 117 aa, synthetic construct
EVQLVESGGGLVQPGGSLTLSCAASGSSVKFLSMAWYRQAPGKKRELVAGISTDGSTDYIDSVKGRFTISRDNAKNSVYLQMNSLRAEDTAVYYCYAYKWTTRYTYWGQGTLVTVSS

SEQ ID NO: 632 - protein, 117 aa, synthetic construct
EVQLVESGGGLVQPGGSLTLSCAASGSSVSFLSSAWYRQAPGKKRELVAGISADGSTLYIDSVKGRFTISRDNAKNSVYLQMNSLRAEDTAVYYCYAYRWTTRSTYWGQGTLVTVSS

SEQ ID NO: 633 - protein, 117 aa, synthetic construct
EVQLVESGGGLVQPGGSLTLSCAASGSSVKFLSMAWYRQAPGKKRELVAGISADGSTDYIDSVKGRFTISRDNAKNSVYLQMNSLRAEDTAVYYCYAYRWRTRYTYWGQGTLVTVSS

SEQ ID NO: 634 - protein, 117 aa, synthetic construct
EVQLVESGGGLVQPGGSLTLSCAASGSSVSFLSMAWYRQAPGKKRELVAGISATGSTDYIDSVKGRFTISRDNAKNSVYLQMNSLRAEDTAVYYCYAYRWSTRYTYWGQGTLVTVSS

SEQ ID NO: 635 - protein, 117 aa, synthetic construct
EVQLVESGGGLVQPGGSLTLSCAASGSTVSFLSMAWYRQAPGKKRELVAGISHDGSTDYIDSVKGRFTISRDNAKNSVYLQMNSLRAEDTAVYYCYAYRWTTRYTYWGQGTLVTVSS

SEQ ID NO: 636 - protein, 117 aa, synthetic construct
EVQLVESGGGLVQPGGSLTLSCAASGSSVQFLSMAWYRQAPGKKRELVAGISYDGSTDYIDSVKGRFTISRDNAKNSVYLQMNSLRAEDTAVYYCYAYRWRTRYTYWGQGTLVTVSS

SEQ ID NO: 637 - protein, 117 aa, synthetic construct
EVQLVESGGGLVQPGGSLTLSCAASRSSVSFLSMAWYRQAPGKKRELVAGISTDGSTDYIDSVKGRFTISRDNAKNSVYLQMNSLRAEDTAVYYCYAYRWLTRYTYWGQGTLVTVSS

SEQ ID NO: 638 - protein, 117 aa, synthetic construct
EVQLVESGGGLVQPGGSLTLSCAASGSKVSFLSMAWYRQAPGKKRELVAGISADGSTAYIDSVKGRFTISRDNAKNSVYLQMNSLRAEDTAVYYCYAYRWRTRYTYWGQGTLVTVSS

SEQ ID NO: 639 - protein, 117 aa, synthetic construct
EVQLVESGGGLVQPGGSLTLSCAASGSRVSFLSMAWYRQAPGKKRELVAGISADGSTDYIESVKGRFTISRDNAKNSVYLQMNSLRAEDTAVYYCYAYRWTTRYTYWGQGTLVTVSS

SEQ ID NO: 640 - protein, 117 aa, synthetic construct
EVQLVESGGGLVQPGGSLTLSCAASGSSVSFLSMAWYRQAPGKKRELVAGISIDGSTDYIKSVKGRFTISRDNAKNSVYLQMNSLRAEDTAVYYCYAYRWTTRYRYWGQGTLVTVSS

SEQ ID NO: 641 - protein, 117 aa, synthetic construct
EVQLVESGGGLVQPGGSLTLSCAASGSKVSFLSMAWYRQAPGKKRELVAGISADGSKDYIDSVKGRFTISRDNAKNSVYLQMNSLRAEDTAVYYCYAYRWTTRYTYWGQGTLVTVSS

SEQ ID NO: 642 - protein, 117 aa, synthetic construct
EVQLVESGGGLVQPGGSLTLSCAASGSSVRFLSMAWYRQAPGKKRELVAGISADGSTVYIDSVKGRFTISRDNAKNSVYLQMNSLRAEDTAVYYCYAYRWPTRYTYWGQGTLVTVSS

SEQ ID NO: 643 - protein, 117 aa, synthetic construct
EVQLVESGGGLVQPGGSLTLSCAASGSSVKFLSMAWYRQAPGKKRELVAGISRDGSTDYIDSVKGRFTISRDNAKNSVYLQMNSLRAEDTAVYYCYAYRWTTRHTYWGQGTLVTVSS

SEQ ID NO: 644 - protein, 117 aa, synthetic construct
EVQLVESGGGLVQPGGSLTLSCAASGSSVKFLSMAWYRQAPGKKRELVAGISADGSTDYIHSVKGRFTISRDNAKNSVYLQMNSLRAEDTAVYYCYAYRWTRRYTYWGQGTLVTVSS

SEQ ID NO: 645 - protein, 117 aa, synthetic construct
EVQLVESGGGLVQPGGSLTLSCAASGSSVSILSMAWYRQAPGKKRELVAGISADGSTIYIDSVKGRFTISRDNAKNSVYLQMNSLRAEDTAVYYCYAYRWHTRYTYWGQGTLVTVSS

SEQ ID NO: 646 - protein, 117 aa, synthetic construct
EVQLVESGGGLVQPGGSLTLSCAASGSSVSFLSVAWYRQAPGKKRELVAGISANGSTDYIDSVKGRFTISRDNAKNSVYLQMNSLRAEDTAVYYCYAYRWTNRYTYWGQGTLVTVSS

SEQ ID NO: 647 - protein, 117 aa, synthetic construct
EVQLVESGGGLVQPGGSLTLSCAASGSSVRFLSMAWYRQAPGKKRELVAGISTDGSTDYIDSVKGRFTISRDNAKNSVYLQMNSLRAEDTAVYYCYAYRWTTRYRYWGQGTLVTVSS

SEQ ID NO: 648 - protein, 117 aa, synthetic construct
EVQLVESGGGLVQPGGSLTLSCAASGSRVSFLSMAWYRQAPGKKRELVAGISYDGSTDYIDSVKGRFTISRDNAKNSVYLQMNSLRAEDTAVYYCYAYRWTTRRTYWGQGTLVTVSS

SEQ ID NO: 649 - protein, 117 aa, synthetic construct
EVQLVESGGGLVQPGGSLTLSCAASGHSVSFLSMAWYRQAPGKKRELVAGISADGSTDYIASVKGRFTISRDNAKNSVYLQMNSLRAEDTAVYYCYAYRWSTRYTYWGQGTLVTVSS

SEQ ID NO: 650 - protein, 117 aa, synthetic construct
EVQLVESGGGLVQPGGSLTLSCAASGSSVRFLSMAWYRQAPGKKRELVAGISADGSTDYIGSVKGRFTISRDNAKNSVYLQMNSLRAEDTAVYYCYAYRWSTRYTYWGQGTLVTVSS

SEQ ID NO: 651 - protein, 117 aa, synthetic construct
EVQLVESGGGLVQPGGSLTLSCAASGSSVSFLSMAWYRQAPGKKRELVAGISANGSTDYYDSVKGRFTISRDNAKNSVYLQMNSLRAEDTAVYYCYAYRWRTRYTYWGQGTLVTVSS

SEQ ID NO: 652 - protein, 117 aa, synthetic construct
EVQLVESGGGLVQPGGSLTLSCAASGSRVSFLSMAWYRQAPGKKRELVAGISADGSTSYIDSVKGRFTISRDNAKNSVYLQMNSLRAEDTAVYYCYAYRWTTRYTYWGQGTLVTVSS

SEQ ID NO: 653 - protein, 117 aa, synthetic construct
EVQLVESGGGLVQPGGSLTLSCAASGSSVKFLSMAWYRQAPGKKRELVAGVSADGSTDYIDSVKGRFTISRDNAKNSVYLQMNSLRAEDTAVYYCYAYEWTTRYTYWGQGTLVTVSS

SEQ ID NO: 654 - protein, 117 aa, synthetic construct
EVQLVESGGGLVQPGGSLTLSCAASGSSVSRLSMAWYRQAPGKKRELVAGISARGSTDYIDSVKGRFTISRDNAKNSVYLQMNSLRAEDTAVYYCYAYRWTTRSTYWGQGTLVTVSS

SEQ ID NO: 655 - protein, 117 aa, synthetic construct
EVQLVESGGGLVQPGGSLTLSCAASGRSVSFLSMAWYRQAPGKKRELVAGISADGSTIYIDSVKGRFTISRDNAKNSVYLQMNSLRAEDTAVYYCYAYRWTTRYTYWGQGTLVTVSS

SEQ ID NO: 656 - protein, 117 aa, synthetic construct
EVQLVESGGGLVQPGGSLTLSCAASGRSVSFLSMAWYRQAPGKKRELVAGISANGSTDYIDSVKGRFTISRDNAKNSVYLQMNSLRAEDTAVYYCYAYRWSTRYTYWGQGTLVTVSS

SEQ ID NO: 657 - protein, 117 aa, synthetic construct
EVQLVESGGGLVQPGGSLTLSCAASGSSVKFLSMAWYRQAPGKKRELVAGISADGSTDYVDSVKGRFTISRDNAKNSVYLQMNSLRAEDTAVYYCYAYRWSTRYTYWGQGTLVTVSS

SEQ ID NO: 658 - protein, 117 aa, synthetic construct
EVQLVESGGGLVQPGGSLTLSCAASGSSVSKLSMAWYRQAPGKKRELVAGISADGSTDYRDSVKGRFTISRDNAKNSVYLQMNSLRAEDTAVYYCYAYRWTYRYTYWGQGTLVTVSS

SEQ ID NO: 659 - protein, 117 aa, synthetic construct
EVQLVESGGGLVQPGGSLTLSCAASGSSVSRLSMAWYRQAPGKKRELVAGISVDGSTDYIDSVKGRFTISRDNAKNSVYLQMNSLRAEDTAVYYCYAYRWTTRLTYWGQGTLVTVSS

SEQ ID NO: 660 - protein, 117 aa, synthetic construct
EVQLVESGGGLVQPGGSLTLSCAASGSSVKFLSLAWYRQAPGKKRELVAGISADGSTDYILSVKGRFTISRDNAKNSVYLQMNSLRAEDTAVYYCYAYEWTTRYTYWGQGTLVTVSS

SEQ ID NO: 661 - protein, 117 aa, synthetic construct
EVQLVESGGGLVQPGGSLTLSCAASGSSVSRLSLAWYRQAPGKKRELVAGISVDGSTDYIDSVKGRFTISRDNAKNSVYLQMNSLRAEDTAVYYCYAYRWTTRLTYWGQGTLVTVSS

SEQ ID NO: 662 - protein, 119 aa, synthetic construct
EVQLVESGGGLVQPGGSLTLSCVASGTSSSINAMGWYRRAPGKQRELVAGISSDGSKVFNESVKGRFTISRDNAKNSVYLQMNSLRAEDTAVYYCYYFRPAAGSPMRYWGQGTLVTVSS

SEQ ID NO: 663 - protein, 119 aa, synthetic construct
EVQLVESGGGLVQPGGSLTLSCVASGTTSSINAIGWYRRAPGKQRELVAGISSDGSEVYTDSVKGRFTISRDNAKNSVYLQMNSLRAEDTAVYYCYYFRTVDGSPLRYWGQGTLVTVSS

SEQ ID NO: 664 - protein, 119 aa, synthetic construct
EVQLVESGGGLVQPGGSLTLSCVASGSTSSINAMAWYRRAPGKQRELVAGISSDDSNVYYESVKGRFTISRDNAKNSVYLQMNSLRAEDTAVYYCYYFRTVSGSSKRYWGQGTLVTVSS

SEQ ID NO: 665 - protein, 119 aa, synthetic construct
EVQLVESGGGLVQPGGSLTLSCVASGQTYRVNAFGWYRRAPGKQRELVAGISSDGSKVYADSVKGRFTISRDNAKNSVYLQMNSLRAEDTAVYYCYYFSAGSGTEMSYWGQGTLVTVSS

SEQ ID NO: 666 - protein, 119 aa, synthetic construct
EVQLVESGGGLVQPGGSLTLSCVASGSTSSINAMAWYRRAPGKQRELVAGISSDESTLYVDSVKGRFTISRDNAKNSVYLQMNSLRAEDTAVYYCYYFGSLSGSSTTYWGQGTLVTVSS

SEQ ID NO: 667 - protein, 119 aa, synthetic construct
EVQLVESGGGLVQPGGSLTLSCVASGSASLTNATGWYRRAPGKQRELVAGISSDDSKVYSDSVKGRFTISRDNAKNSVYLQMNSLRAEDTAVYYCYYFGSVSGSWTRYWGQGTLVTVSS

SEQ ID NO: 668 - protein, 119 aa, synthetic construct
EVQLVESGGGLVQPGGSLTLSCVASGYPSLNNAMGWYRRAPGKQRELVAGISSDGSQVYGASVKGRFTISRDNAKNSVYLQMNSLRAEDTAVYYCYYFRLVSGSSMSYWGQGTLVTVSS

SEQ ID NO: 669 - protein, 119 aa, synthetic construct
EVQLVESGGGLVQPGGSLTLSCVASGSSSTINAIGWYRRAPGKQRELVAGISSDGSKVYADSVKGRFTISRDNAKNSVYLQMNSLRAEDTAVYYCYYFRTGSGTSKSYWGQGTLVTVSS

SEQ ID NO: 670 - protein, 119 aa, synthetic construct
EVQLVESGGGLVQPGGSLTLSCVASGSTSYINAMGWYRRAPGKQRELVAGISSDGSNMYADSVKGRFTISRDNAKNSVYLQMNSLRAEDTAVYYCYYFSNMSGTTRRYWGQGTLVTVSS

SEQ ID NO: 671 - protein, 119 aa, synthetic construct
EVQLVESGGGLVQPGGSLTLSCVASGSTSSVNALGWYRRAPGKQRELVAGISSDGSKVYTDSVKGRFTISRDNAKNSVYLQMNSLRAEDTAVYYCYYFRTVPGSAMGYWGQGTLVTVSS

SEQ ID NO: 672 - protein, 119 aa, synthetic construct
EVQLVESGGGLVQPGGSLTLSCVASGSTSLSNAVGWYRRAPGKQRELVAGISSDGSKVSAESVKGRFTISRDNAKNSVYLQMNSLRAEDTAVYYCYYFRAESGSSMGYWGQGTLVTVSS

SEQ ID NO: 673 - protein, 119 aa, synthetic construct
EVQLVESGGGLVQPGGSLTLSCVASGSTSSTNAIGWYRRAPGKQRELVAGISSDGSKVYDDSVKGRFTISRDNAKNSVYLQMNSLRAEDTAVYYCYYFRTLYGSSRSYWGQGTLVTVSS

SEQ ID NO: 674 - protein, 119 aa, synthetic construct
EVQLVESGGGLVQPGGSLTLSCVASGLTSTINAMGWYRRAPGKQRELVAGISSDGSKVYDDSVKGRFTISRDNAKNSVYLQMNSLRAEDTAVYYCYYFSPFSGSDTGYWGQGTLVTVSS

SEQ ID NO: 675 - protein, 119 aa, synthetic construct
EVQLVESGGGLVQPGGSLTLSCVASGVSPSKNAIGWYRRAPGKQRELVAGISSDGSAVYVGSVKGRFTISRDNAKNSVYLQMNSLRAEDTAVYYCYYFSTFSGSSISYWGQGTLVTVSS

SEQ ID NO: 676 - protein, 119 aa, synthetic construct
EVQLVESGGGLVQPGGSLTLSCVASGSTSSINAVGWYRRAPGKQRELVAGISSDGSYVYSESVKGRFTISRDNAKNSVYLQMNSLRAEDTAVYYCYYFRTLAGSEMRYWGQGTLVTVSS

SEQ ID NO: 677 - protein, 119 aa, synthetic construct
EVQLVESGGGLVQPGGSLTLSCVASGSTTMNNAMAWYRRAPGKQRELVAGISSDSSHVYADSVKGRFTISRDNAKNSVYLQMNSLRAEDTAVYYCYYFRTVSGSGVRYWGQGTLVTVSS

SEQ ID NO: 678 - protein, 119 aa, synthetic construct
EVQLVESGGGLVQPGGSLTLSCVASGSTSKINAIGWYRRAPGKQRELVAGISSDSSIVYTDSVKGRFTISRDNAKNSVYLQMNSLRAEDTAVYYCYYFRPGAGHSNSYWGQGTLVTVSS

SEQ ID NO: 679 - protein, 119 aa, synthetic construct
EVQLVESGGGLVQPGGSLTLSCVASGQTTALNAMGWYRRAPGKQRELVAGISSDGSEVNTDSVKGRFTISRDNAKNSVYLQMNSLRAEDTAVYYCYYFRRASGTAMSYWGQGTLVTVSS

SEQ ID NO: 680 - protein, 119 aa, synthetic construct
EVQLVESGGGLVQPGGSLTLSCVASGATSSINAIGWYRRAPGKQRELVAGISSDGSKLSSDSVKGRFTISRDNAKNSVYLQMNSLRAEDTAVYYCYYFTSASGTDLSYWGQGTLVTVSS

SEQ ID NO: 681 - protein, 119 aa, synthetic construct
EVQLVESGGGLVQPGGSLTLSCVASGSTSTINAMGWYRRAPGKQRELVAGISSDNSKVYADSVKGRFTISRDNAKNSVYLQMNSLRAEDTAVYYCYYFRSANGSSKRYWGQGTLVTVSS

SEQ ID NO: 682 - protein, 119 aa, synthetic construct
EVQLVESGGGLVQPGGSLTLSCVASGSTSSINAMGWYRRAPGKQRELVAGISSDGSRVYFDSVKGRFTISRDNAKNSVYLQMNSLRAEDTAVYYCYYFKTIAGAGMRYWGQGTLVTVSS

SEQ ID NO: 683 - protein, 119 aa, synthetic construct
EVQLVESGGGLVQPGGSLTLSCVASGSTSLVNAMGWYRRAPGKQRELVAGISSDGSLVYAESVKGRFTISRDNAKNSVYLQMNSLRAEDTAVYYCYYFRYGSGSSLSYWGQGTLVTVSS

SEQ ID NO: 684 - protein, 119 aa, synthetic construct
EVQLVESGGGLVQPGGSLTLSCVASGSTSLNNAIGWYRRAPGKQRELVAGISSDGSVVYVDSVKGRFTISRDNAKNSVYLQMNSLRAEDTAVYYCYYFRTVPGASMKYWGQGTLVTVSS

SEQ ID NO: 685 - protein, 119 aa, synthetic construct
EVQLVESGGGLVQPGGSLTLSCVASGSTSPVNAMAWYRRAPGKQRELVAGISSDGSKVYVDSVKGRFTISRDNAKNSVYLQMNSLRAEDTAVYYCYYFRTVDGSAISYWGQGTLVTVSS

SEQ ID NO: 686 - protein, 119 aa, synthetic construct
EVQLVESGGGLVQPGGSLTLSCVASGTTSSMNAIGWYRRAPGKQRELVAGISSDGSKLYDESVKGRFTISRDNAKNSVYLQMNSLRAEDTAVYYCYYFRTVKGSGGSYWGQGTLVTVSS

SEQ ID NO: 687 - protein, 119 aa, synthetic construct
EVQLVESGGGLVQPGGSLTLSCVASGETSSINAMAWYRRAPGKQRELVAGISSDYSKLYADSVKGRFTISRDNAKNSVYLQMNSLRAEDTAVYYCYYFRTVSGSSRGYWGQGTLVTVSS

SEQ ID NO: 688 - protein, 119 aa, synthetic construct
EVQLVESGGGLVQPGGSLTLSCVASGSTSTINAIGWYRRAPGKQRELVAGISSDSSKVYTESVKGRFTISRDNAKNSVYLQMNSLRAEDTAVYYCYYFRPGPGSQMAYWGQGTLVTVSS

SEQ ID NO: 689 - protein, 119 aa, synthetic construct
EVQLVESGGGLVQPGGSLTLSCVASGSTYSMNAMGWYRRAPGKQRELVAGISSDGSQVYVDSVKGRFTISRDNAKNSVYLQMNSLRAEDTAVYYCYYFRTVAGSASGYWGQGTLVTVSS

SEQ ID NO: 690 - protein, 119 aa, synthetic construct; identical to SEQ ID NO: 521

SEQ ID NO: 691 - protein, 119 aa, synthetic construct
EVQLVESGGGLVQPGGSLTLSCVASGSTSTINAIGWYRRAPGKQRELVAGISSDGSKVYVDSVKGRFTISRDNAKNSVYLQMNSLRAEDTAVYYCYYFINLKGSSMAYWGQGTLVTVSS

SEQ ID NO: 692 - protein, 119 aa, synthetic construct
EVQLVESGGGLVQPGGSLTLSCVASGSTSSINAIGWYRRAPGKQRELVAGISSDGSKVYADSVKGRFTISRDNAKNSVYLQMNSLRAEDTAVYYCYYFRMVTGSYGGYWGQGTLVTVSS

SEQ ID NO: 693 - protein, 119 aa, synthetic construct
EVQLVESGGGLVQPGGSLTLSCVASGSISSINAMGWYRRAPGKQRELVAGISSDGSSVYADSVKGRFTISRDNAKNSVYLQMNSLRAEDTAVYYCYYFKSSYGLPMRYWGQGTLVTVSS

SEQ ID NO: 694 - protein, 119 aa, synthetic construct
EVQLVESGGGLVQPGGSLTLSCVASGSTQVNNAMAWYRRAPGKQRELVAGISSDGSQVYYGSVKGRFTISRDNAKNSVYLQMNSLRAEDTAVYYCYYFKTVSGQSLRYWGQGTLVTVSS

SEQ ID NO: 695 - protein, 119 aa, synthetic construct
EVQLVESGGGLVQPGGSLTLSCVASGSTASFNAMAWYRRAPGKQRELVAGISSDGSKVYTDSVKGRFTISRDNAKNSVYLQMNSLRAEDTAVYYCYYFRTVTGRAARYWGQGTLVTVSS

SEQ ID NO: 696 - protein, 119 aa, synthetic construct
EVQLVESGGGLVQPGGSLTLSCVASGSPLSINAIGWYRRAPGKQRELVAGISSDGSKVSADSVKGRFTISRDNAKNSVYLQMNSLRAEDTAVYYCYYFGPAIGASRTYWGQGTLVTVSS

SEQ ID NO: 697 - protein, 119 aa, synthetic construct
EVQLVESGGGLVQPGGSLTLSCVASGSTTFINAIGWYRRAPGKQRELVAGISSDGSKVYEDSVKGRFTISRDNAKNSVYLQMNSLRAEDTAVYYCYYFRTVSGAPKSYWGQGTLVTVSS

SEQ ID NO: 698 - protein, 119 aa, synthetic construct
EVQLVESGGGLVQPGGSLTLSCVASGSTSSINAIGWYRRAPGKQRELVAGISSDRSKVYADSVKGRFTISRDNAKNSVYLQMNSLRAEDTAVYYCYYFHTVSGSSMSYWGQGTLVTVSS

SEQ ID NO: 699 - protein, 119 aa, synthetic construct
EVQLVESGGGLVQPGGSLTLSCVASGETDTINAVGWYRRAPGKQRELVAGISSDGSKVYAESVKGRFTISRDNAKNSVYLQMNSLRAEDTAVYYCYYFRRLEGYSNRYWGQGTLVTVSS

SEQ ID NO: 700 - protein, 119 aa, synthetic construct
EVQLVESGGGLVQPGGSLTLSCVASGSTSPINAIGWYRRAPGKQRELVAGISSDGSVVTTESVKGRFTISRDNAKNSVYLQMNSLRAEDTAVYYCYYFRTGSGSSMGYWGQGTLVTVSS

SEQ ID NO: 701 - protein, 119 aa, synthetic construct
EVQLVESGGGLVQPGGSLTLSCVASGSITSSNAMGWYRRAPGKQRELVAGISSDGSHVHQESVKGRFTISRDNAKNSVYLQMNSLRAEDTAVYYCYYFTTVTGSSMSYWGQGTLVTVSS

SEQ ID NO: 702 - protein, 117 aa, synthetic construct
EVQLVESGGGLVQPGGSLTLSCAASRYSVSNLSMAWYRQAPGKKRELVAGISADGSTVYVESVKGRFTISRDNAKNSVYLQMNSLRAEDTAVYYCYAYYWTERRPYWGQGTLVTVSS

SEQ ID NO: 703 - protein, 117 aa, synthetic construct
EVQLVESGGGLVQPGDSLTLSCAASMSTVSVLSMAWYRQAPGKKRELVAGISSDGSTVYIDSVKGRFTISRDNAKNSVYLQMNSLRAEDTAIYYCYAYSWDDAHPYWGQGTLVTVSS

SEQ ID NO: 704 - protein, 117 aa, synthetic construct
EVQLVESGGGLVQPGGSLTLSCAASDSYVSLLSMAWYRQAPGKKRELVAGISVDGSTHYVASVKGRFTISRDNAKNSVYLQMNSLRAEDTAVYYCYAYRWMTRLTYWGQGTLVTVSS

SEQ ID NO: 705 - protein, 117 aa, synthetic construct
EVQLVESGGGLVQPGGSLTLSCAASDSAVSVLSIAWYRQAPGKKRELVAGISTDGSKHYIDSVKGRFTISRDNAKNSVYLQMNSLRAEDTAVYYCYAYDWADAQPYWGQGTLVTVSS

SEQ ID NO: 706 - protein, 117 aa, synthetic construct
EVQLVESGGGLVQPGGSLTLSCAASHSSVTSLSLAWYRQAPGKKRELVAGISYDGSKYYAESVKGRFTISRDNAKNSVYLQMNSLRAEDTAVYYCYAYSWTDRLPYWGQGTLVTVSS

SEQ ID NO: 707 - protein, 117 aa, synthetic construct
EVQLVESGGGLVQPGGSLTLSCAASDSVVKFLSMAWYRQAPGKKRELVAGISANGSRTYMESVKGRFTISRDNAKNSVYLQMNSLRAEDTAVYYCYAYRWATRLPYWGQGTLVTVSS

SEQ ID NO: 708 - protein, 117 aa, synthetic construct
EVQLVESGGGLVQPGGSLTLSCAASDPSVWNLSMAWYRQAPGKKRELVAGISPDGSTDYVDSVKGRFTISRDNAKNSVYLQMNSLRAEDTAVYYCYAYKWSNRLPYWGQGTLVTVSS

SEQ ID NO: 709 - protein, 117 aa, synthetic construct
EVQLVESGGGLVQPGGSLTLSCAASGTSVMLLSLAWYRQAPGKKRELVAGISPNGSAVYTESVKGRFTISRDNAKNSVYLQMNSLRAEDTAVYYCYAYGWKTRQPYWGQGTLVTVSS

SEQ ID NO: 710 - protein, 117 aa, synthetic construct
EVQLVESGGGLVQPGGSLTLSCAASSSPVSNLSLAWYRQAPGKKRELVAGISPDGSTAYMESVKGRFTISRDNAKNSVYLQMNSLRAEDTAVYYCYAYRWPNRRGYWGQGTLVTVSS

SEQ ID NO: 711 - protein, 117 aa, synthetic construct
EVQLVESGGGLVQPGGSLTLSCAASWRSVLLLSVAWYRQAPGKKRELVAGISNDGSTDYIDSVKGRFTISRDNAKNSVYLQMNSLRAEDTAVYYCYAYDWTTRQRYWGQGTLVTVSS

SEQ ID NO: 712 - protein, 117 aa, synthetic construct
EVQLVESGGGLVQPGGSLTLSCAASSSSVQYLSMAWYRQAPGKKRELVAGISTDGSAVYFDSVKGRFTISRDNAKNSVYLQMNSLRAEDTAVYYCYAYNWSYAQPYWGQGTLVTVSS

SEQ ID NO: 713 - protein, 117 aa, synthetic construct
EVQLVESGGGLVQPGGSLTLSCAASGTSVSLLSLAWYRQAPGKKRELVAGISTGGSTHYIESVKGRFTISRDNAKNSVYLQMNSLRAEDTAVYYCYAYNWTDSLQYWGQGTLVTVSS

SEQ ID NO: 714 - protein, 117 aa, synthetic construct
EVQLVESGGGLVQPGGSLTLSCAASLSSVSNLSIAWYRQAPGKKRELVAGISTDGSTVYIDSVKGRFTISRDNAKNSVYLQMNSLRAEDTAVYYCYAYSWTTSLPYWGQGTLVTVSS

SEQ ID NO: 715 - protein, 117 aa, synthetic construct
EVQLVESGGGLVQPGGSLTLSCAASMYSVSFLSMAWYRQAPGKKRELVAGISNEGSTYYMDSVKGRFTISRDNAKNSVYLQMNSLRAEDTAVYYCYAYKWRSRSTYWGQGTLVTVSS

SEQ ID NO: 716 - protein, 117 aa, synthetic construct
EVQLVESGGGLVQPGGSLTLSCAASKSSVSHLSLAWYRQAPGKKRELVAGISADGSHVYTNSVKGRFTISRDNAKNSVYLQMNSLRAEDTAVYYCYAYSQTTRDPYWGQGTLVTVSS

SEQ ID NO: 717 - protein, 117 aa, synthetic construct
EVQLVESGGGLVQPGGSLTLSCAASYTSVLDLSIAWYRQAPGKKRELVAGISDDGSRYYTDSVKGRFTISRDNAKNSVYLQMNSLRAEDTAVYYCYAYRWTARDTYWGQGTLVTVSS

SEQ ID NO: 718 - protein, 117 aa, synthetic construct
EVQLVESGGGLVQPGGSLTLSCAASMSDVSFLSMAWYRQAPGKKRELVAGISAEGSTLYMESVKGRFTISRDNAKNSVYLQMNSLRAEDTAVYYCYAYRWTSRLSYWGQGTLVTVSS

SEQ ID NO: 719 - protein, 117 aa, synthetic construct
EVQLVESGGGLVQPGGSLTLSCAASESSVSFLSSAWYRQAPGKKRELVAGISTDGSTVYIDSVKGRFTISRDNAKNSVYLQMNSLRAEDTAVYYCYAYSWTTRSRYWGQGTLVTVSS

SEQ ID NO: 720 - protein, 117 aa, synthetic construct
EVQLVESGGGLVQPGGSLTLSCAASGDSVSLLSMAWYRQAPGKKRELVAGISANGSTSYIDSVKGRFTISRDNAKNSVYLQMNSLRAEDTAVYYCYAYNWTSRYRYWGQGTLVTVSS

SEQ ID NO: 721 - protein, 117 aa, synthetic construct
EVQLVESGGGLVQPGGSLTLSCAASGSDVWYLSLAWYRQAPGKKRELVAGISDDGSRHYIESVKGRFTISRDNAKNSVYLQMNSLRAEDTAVYYCYAYSWKTRFPYWGQGTLVTVSS

SEQ ID NO: 722 - protein, 117 aa, synthetic construct
EVQLVESGGGLVQPGGSLTLSCAASKSAVAFLSIAWYRQAPGKKRELVAGISPDGSTVYIESVKGRFTISRDNAKNSVYLQMNSLRAEDTAVYYCYAYSWTTRYPYWGQGTLVTVSS

SEQ ID NO: 723 - protein, 117 aa, synthetic construct
EVQLVESGGGLVQPGGSLTLSCAASFSAVAYLSMAWYRQAPGKKRELVAGISDDGSTVYVDSVKGRFTISRDNAKNSVYLQMNSLRAEDTAVYYCYAYEWTNALPYWGQGTLVTVSS

SEQ ID NO: 724 - protein, 117 aa, synthetic construct
EVQLVESGGGLVQPGGSLTLSCAASVYSVYDLSTAWYRQAPGKKRELVAGISDDGSTVYFDSVKGRFTISRDNAKNSVYLQMNSLRAEDTAVYYCYAYSWITRSPYWGQGTLVTVSS

SEQ ID NO: 725 - protein, 117 aa, synthetic construct
EVQLVESGGGLVQPGGSLTLSCAASGDSVSFLSMAWYRQAPGKKRELVAGISDEGSTVYIGSVKGRFTISRDNAKNSVYLQMNSLRAEDTAVYYCYAYSWTTRRQYWGQGTLVTVSS

SEQ ID NO: 726 - protein, 117 aa, synthetic construct
EVQLVESGGGLVQPGGSLTLSCAASSSSVSLLSLAWYRQAPGKKRELVAGISDDGSIVYMDSVKGRFTISRDNAKNSVYLQMNSLRAEDTAVYYCYAYSWITRSPYWGQGTLVTVSS

SEQ ID NO: 727 - protein, 117 aa, synthetic construct
EVQLVESGGGLVQPGGSLTLSCAASADSVSFLSIAWYRQAPGKKRELVAGISDDGSKHYFDSVKGRFTISRDNAKNSVYLQMNSLRAEDTAVYYCYAYRWEESRQYWGQGTLVTVSS

SEQ ID NO: 728 - protein, 117 aa, synthetic construct
EVQLVESGGGLVQPGGSLTLSCAASASSVTLLSIAWYRQAPGKKRELVAGISTDGSTDYLHSVKGRFTISRDNAKNSVYLQMNSLRAEDTAVYYCYAYTWTTRLPYWGQGTLVTVTS

SEQ ID NO: 729 - protein, 117 aa, synthetic construct; identical to SEQ ID NO: 727

SEQ ID NO: 730 - protein, 117 aa, synthetic construct
EVQLVESGGGLVQPGGSLTLSCAASGTSVWLLSMAWYRQAPGKKRELVAGISYDGSTVYVESVKGRFTISRDNAKNSVYLQMNSLRAEDTAVYYCYAYSWTTRQPYWGQGTLVTVSS

SEQ ID NO: 731 - protein, 117 aa, synthetic construct
EVQLVESGGGLVQPGGSLTLSCAASGSSVSILSIAWYRQAPGKKRELVAGISDDGSTVYIDSVKGRFTISRDNAKNSVYLQMNSLRAEDTAVYYCYAYVWGTRLPYWGQGTLVTVSS

SEQ ID NO: 732 - protein, 117 aa, synthetic construct
EVQLVESGGGLVQPGGSLTLSCAASGTAVSNLSIAWYRQAPGKKRELVAGISDDGSTVYVDSVKGRFTISRDNAKNSVYLQMNSLRAEDTAVYYCYAYEWTNALPYWGQGTLVTVSS

SEQ ID NO: 733 - protein, 117 aa, synthetic construct
EVQLVESGGGLVQPGGSLTLSCAASGSAVSMLSLAWYRQAPGKKRELVAGISDDGSQVYIDSVKGRFTISRDNAKNSVYLQMNSLRAEDTAVYYCYAYRWEDALTYWGQGTLVTVSS

SEQ ID NO: 734 - protein, 117 aa, synthetic construct
EVQLVESGGGLVQPGGSLTLSCAASGMTVFFLSMAWYRQAPGKKRELVAGISVDGSTVYSDSVKGRFTISRDNAKNSVYLQMNSLRAEDTAVYYCYAYSWTTRYPYWGQGTLVTVSS

SEQ ID NO: 735 - protein, 117 aa, synthetic construct
EVQLVESGGGLVQPGGSLTLSCAASQYSVTFLSVAWYRQAPGKKRELVAGISDDGSNVYIDSVKGRFTISRDNAKNSVYLQMNSLRAEDTAVYYCYAYSWIDSLRYWGQGTLVTVSS

SEQ ID NO: 736 - protein, 117 aa, synthetic construct
EVQLVESGGGLVQPGGSLTLSCAASGETVSFLSLAWYRQAPGKKRELVAGISTDGSTVYFVSVKGRFTISRDNAKNSVYLQMNSLRAEDTAVYYCYAYSWTTPRAYWGQGTLVTVSS

SEQ ID NO: 737 - protein, 121 aa, synthetic construct
QVQLQESGGGLVQAGGSLRLSCAASGSIAYIYTMDWYRQAPGKQRELVATSTRDGNVDYAESVKGRFTISRDNAKNTVYLQMNSLKPEDTAVYYCNADLRTAVDLIRANYWGQGTQVTVSS

SEQ ID NO: 738 - protein, 125 aa, synthetic construct
QVQLQESGGGLVQAGDSLRLSCVVSGRTDSWYVMGWFRQAPGKDREFVAGVSWSYGNTYYADSVKGRFTASRDNAKNTAYLQMNSLNAEDTAVYYCAARVSREVIPTRWDLYNYWGQGTQVTVSS

SEQ ID NO: 739 - protein, 114 aa, synthetic construct
QVQLQESGGGSVQPGGSLRVSCVVSRTIISINAMTWYHQAPGKRRELVAIITSGGETNYADSVKGRFTISRDNAKNTAYLQMNNLKPEDTGVYYCNVVPPLGSWGQGTQVTVSS

SEQ ID NO: 740 - protein, 127 aa, synthetic construct
QVQLQESGGGRVQAGGSLRLSCSASARTLRLYAVGWFRQAPGKEREFVAGIGRSERTYYTDSVKGRFTLSRDNAKNTVFLEMNDLEPEDTAVYFCALTFQTTDMVDVPTTQHEYDYWGRGTQVTVSS

SEQ ID NO: 741 - protein, 121 aa, synthetic construct
QVQLQESGGGLVQAGGSLRLSCAASGSIAYIYTMDWYRQAPGKQRELVATSTHDGNTDYADSVKGRFTISRDNVKNTVYLQMNSLKPEDTAVYYCNADLRTAVDLIRANYWGQGTQVTVSS

SEQ ID NO: 742 - protein, 121 aa, synthetic construct
QVQLQESGGGLVQPGGSLRLSCAASGSIAYIYTMDWYRQAPGKQRELVATSTRDGNTDYADSVKGRFTISRDNAKDTVYLQMNSLKPEDTAVYYCNADLRTAVDLIRANYWGQGTQVTVSS

SEQ ID NO: 743 - protein, 119 aa, synthetic construct
QVQLQESGGGLVQAGGSLTLSCAASGRYQMAWFRQAPEKEREFVGTISSGDSTWYTNSVKGRFAISRDSARNTVYLQMNDLKPEDTAIYYCAAALYYRDSRRAADYPYWGQGTQVTVSS

SEQ ID NO: 744 - protein, 127 aa, synthetic construct
QVQLQESGGGRVQAGESLRLSCSTSTRTLKLYAVGWFRQAPGKERDFVAGIGRSERIYYIDSVKGRFTLSRDNAKNTVFLEMNDLEPEDTAVYFCAATFQTSDNVGVPTVQHEYDYWGQGTQVTVSS

SEQ ID NO: 745 - protein, 121 aa, synthetic construct
QVQLQESGGGLVQAGGSLRLSCAASGSIAYIYTMDWYRQAPGKQRELVATSTHDGNTDYADSVKGRFTISRDNAKNTVTLQMNSLKPEDTAVYYCNADLRTAVDLIRANYWGQGTQVTVSS

SEQ ID NO: 746 - protein, 121 aa, synthetic construct
QVQLQESGGGLVQAGGSLRLSCAASGSIAYIYTMDWYRQAPGKQRELVATFTRDGNTDYADSVKGRFTISRDNAKNTVYLQMNSLKPEDTAVYYCNTDLRTAVDLIRANYWGQGTQVTVSS

SEQ ID NO: 747 - protein, 121 aa, synthetic construct
QVQLQESGGGLVQAGGSLRLSCAASGSIAYIYTMDWYRQAPGKQRELVATSTHDGNTDYADSLKGRFTISRDNAKNTVYLQMNSLKPEDTAVYYCNADLRTAVDLIRANYWGQGTQVTVSS

SEQ ID NO: 748 - protein, 119 aa, synthetic construct
QVQLQESGGGSVQAGGSLRLSCAASGRYQMAWFRQAPEKEREFVGTISSGDSTWYTNSLKGRFAISRDSARDTVYLQMNDLKPEDTAVYYCAAALYYRDSRRAADYPYWGQGTQVTVSS

SEQ ID NO: 749 - protein, 114 aa, synthetic construct
QVQLQESGGGLVQTGGSLRLSCAVSGSIVTINAMTWYRQAPGKRRELVAIITSGGETNYADSVKGRFTISRDNAKNTAHLQMNSLNPEDTGVYYCNVVPPLGSWGQGTQVTVSS

SEQ ID NO: 750 - protein, 114 aa, synthetic construct
QVQLQESGGGLVQTGGSLRLSCAVSRSIVSIKSMTWYRQAPGKRRELVALITSGGETNYSDSVKGRFTISRDNAKNTVYLQMNSLKPEDTGVYYCNVVPPLGSWGQGTQVTVSS

SEQ ID NO: 751 - protein, 125 aa, synthetic construct
QVQLQESGGGLVETGGSLRLSCAGSGSTFRHHAMAWFRQTPGKEREFVSAINDHGDRTKYLDSVRGRFTISRDNTDNMVYLQMTDLRPEDTANYSCAAGPLVDYLETTPLVYTYWGHGTQVTVSS

SEQ ID NO: 752 - protein, 121 aa, synthetic construct
QVQLQESGGGLVQAGGSLRLSCAASGSIAYIYTMDWYRQAPGKQRELVATSTHDGNTDYADSVKGRFTISRDNAKNTVYLQMNSLKPEDTAVYYCNADLRTAVDLIRANYWGQGTQVTVSS

SEQ ID NO: 753 - protein, 121 aa, synthetic construct
QVQLQESGGGSVQAGGSLTLSCAASGSIAYIYTMDWYRQAPGKQRELVATSTHDGNTDYADSVKGRFTISRDNAKNTVYLQMNSLKPEDTAVYYCNADLRTAVDLIRANYWGQGTQVTVSS

SEQ ID NO: 754 - protein, 113 aa, synthetic construct
QVQLQESGGGLVQPGGSLRLSCTASVSIFSVNAVDWYRQSPGKERELVAIMTSDGSTNYGDSVKGRFTISRDNAKNTVYLQMNNLKPEDTAVYYCNTVPPRYWGQGTQVTVSS

SEQ ID NO: 755 - protein, 121 aa, synthetic construct
QVQLQESGGGLVQPGGSLRLSCAASGSIAYIYTMDWYRQAPGKQRELVATSTRDGNIDYADSVKGRFTISRDSAKNTVYLQMSSLKPEDTAVYYCNADLRTAVDLIRANYWGQGTQVTVSS

SEQ ID NO: 756 - protein, 119 aa, synthetic construct
QVQLQESGGGLVQAGGSLTLSCAASGRYQLAWFRQAPEKVREFVGTISSGDSTWYTNSVKGRFAISRDSARNTVYLQMNDLKPEDTAVYYCAAALYYRDSRRAADYPYWGQGTQVTVSS

SEQ ID NO: 757 - protein, 119 aa, synthetic construct
QVQLQESGGGSVQAGGSLRLSCAASGRYHMAWFRQAPEKEREFVGTISSGDSTWYTNSVKGRFAISRDSARNTAYLQMNDLKPEDTAVYYCAAALYYGDSRRAADYPYWGQGTQVTVSS

SEQ ID NO: 758 - protein, 121 aa, synthetic construct
QVQLQESGGGSVQAGGSLRLSCAASGSIAYIYTMDWYRQAPGKQRELVATSTHDGNTDYTDSVKGRFTISRDNAKNTVYLQMNSLKPEDTAVYYCNADLRTAVDLIRANYWGQGTQVTVSS

SEQ ID NO: 759 - protein, 121 aa, synthetic construct
QVQLQESGGGLVQAGGSLRLSCAASGSIAYIYTMDWYRQTPGKQRELVATSTRDANTDYAGSVKGRFTISRDNAKDTVYLQMNSLKPEDTAVYYCHADLRTAVDLIRANYWGQGTQVTVSS

SEQ ID NO: 760 - protein, 119 aa, synthetic construct
QVQLQESGGGLVQAGGSLRLSCAASGRYNMAWFRQAPEKEREFVGTITSADSTWYTNSVKGRFAITQDSARNTVYLQMNDLKPEDTAVYYCAAALYYGDSRRAADYPYWGQGTQVTVSS

SEQ ID NO: 761 - protein, 119 aa, synthetic construct
QVQLQESGGGLVQPGGSLRLSCAASGRYQMAWFRQAPEKEREFVGTISSGDSTWYTNSVKGRFAISRDSARTTVYLQMNDLKPEDTAVYYCAAALYYRDSWRAADYPYWGQGTQVTVSS

SEQ ID NO: 762 - protein, 119 aa, synthetic construct
QVQLQESGGGLVQPGESLRLSCAATGRYHLAWFRQAPEKEREFVGTITSADSTWYTNSVKGRFAITRDSARNTVYLQMNDLKPEDTAVYYCAAALYYGDSRRAADYPYWGQGTQVTVSS

SEQ ID NO: 763 - protein, 124 aa, synthetic construct
QVQLQESGGGLVQAGGSLKLSCADSGRSFSNYIMGWFRQAPGKEREFVAGLGWSPGNTYYADSVKGRFTISRDNAKNMVYLQMNSLNPEDTAVYYCAARRGDVIYTTPWNYVYWGQGTQVTVSS

SEQ ID NO: 764 - protein, 121 aa, synthetic construct
QVQLQESGGGLVQAGGSLRLSCAASGSIAYIYTMDWYRQAPGKQRELVATSTHDGNTDYADSVKGRFTISRDNAKNTVYLQMNSLKPEDTAVYYCNADLRTPVDLIRANYWGQGTQVTVSS

SEQ ID NO: 765 - protein, 119 aa, synthetic construct
QVQLQESGGGSVQAGGSLRLSCAAPGRYQMAWFRQAPEKEREFVGTISSGDSTWYTNSVKGRFAISRDSARNTVYLQMNDLKSEDTAVYYCAAALYYRDSRRAIDYPYWGQGTQVTVSS

SEQ ID NO: 766 - protein, 122 aa, synthetic construct
QVQLQESGGGSVQAGGSLRLSCAASGLTFSSYAMAWFRQAPGKQRELVARITSGGTTDYADSVKGRFTISRDNAKNTMYLQMNSLKPEDTAVYYCAADLTYRNLLLKLPHYWGQGTQVTVSS

SEQ ID NO: 767 - protein, 121 aa, synthetic construct
QVQLQESGGGLVQAGGSLRLSCAASGNIAYIYTMDWYRQAPGKQRELVATSTHDGSTDYADSVKGRFTISRDNAKNTVYLQMNSLKPEDTAVYYCNADLRTPVDLIRANYWGQGTQVTVSS

SEQ ID NO: 768 - protein, 121 aa, synthetic construct
QVQLQESGGGLVQPGGSLRLSCAASGSIAYIYTMDWYRQAPGKQRELVATSTWDGNTDYADSVKGRFTISRDNAKNTVYLQMNSLKPEDTAVYYCNADLRTAVDLIRANYWGQGTQVTVSS

SEQ ID NO: 769 - protein, 121 aa, synthetic construct
QVQLQESGGGLVQAGGSLRLSCAASGSIAYIYTMDWYRQAPGKQRELVATSTHDGNTDYADSVKGRFTISRDNAKNTVYLQMSSLKPDDTAVYYCNADLRTAVDLIRANYWGQGTQVTVSS

SEQ ID NO: 770 - protein, 123 aa, synthetic construct
QVQLQESGGGLVQAGESLSLSCAASGNDFVITDMHWYRQAPGKQREWVATITRFATTNYADSVKGRFTISRDNAKNTWYLQMNSLKPDDTAVYYCKAIGLRGVPDVNRQFEVWGQGTQVTVSS

SEQ ID NO: 771 - protein, 121 aa, synthetic construct
QVQLQESGGGLVQAGGSLRLSCAASGAIAYIYGMGWYRQAPGNQRELVAAISSGGSTDYADSVKGRFTISRDNAKNTVYLQMSSLKPEDTAVYYCNADVRTSRNLVRSDYWGQGTQVTVSS

SEQ ID NO: 772 - protein, 119 aa, synthetic construct
QVQLQESGGGLVQAGGSLRLSCAASGRYHTAWFRQAPEKEREFVGTISSGDSTWYTNSVKGRFAISRDSARNTVYLQMNDLKPEDTAVYYCAAALYYGDSRRAGDYPYWGQGTQVTVSS

SEQ ID NO: 773 - protein, 121 aa, synthetic construct
QVQLQESGGGLVQAGGSLRLSCAASGNIAYIYTMNWYRQAPGKQRELVATSTHAGNTDYADSVKGRFTISRDNAKNTVYLQMNSLKPEDTAVYYCNVDLRTAVDLIRANYWGQGTQVTVSS

SEQ ID NO: 774 - protein, 121 aa, synthetic construct
QVQLQESGGGLVQPGGSLRLSCAASGNIAYIYTMGWYRQAPGKQRELVATSTHDGNSDYADSVKGRFTISRDNAKNTVYLQMNTLKPDDTAVYYCNADLRTPVDRIRGNFWGQGTQVTVSS

SEQ ID NO: 775 - protein, 121 aa, synthetic construct
EVQLLESGGGLVQPGGSLTLSCAASGSIAYIYTMDWYRQAPGKQRELVATSTRDGNVDYADSVKGRFTISRDNSKNTLYLQMNSLRAEDTAVYYCNADLRTAVDLIRANYWGLGTQVTVSS

SEQ ID NO: 776 - protein, 125 aa, synthetic construct
EVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSS

SEQ ID NO: 777 - protein, 127 aa, synthetic construct
EVQLLESGGGLVQPGGSLTLSCAASARTLRLYAVGWFRQAPGKEREFVAGIGRSERTYYTDSVKGRFTISRDNSKNTLYLQMNSLRAEDTAVYYCALTFQTTDMVDVPTTQHEYDYWGLGTQVTVSS

SEQ ID NO: 778 - protein, 114 aa, synthetic construct
EVQLLESGGGLVQPGGSLTLSCAASGSIVTINAMTWYRQAPGKRRELVAIITSGGETNYADSVKGRFTISRDNSKNTLYLQMNSLRAEDTAVYYCNVVPPLGSWGLGTQVTVSS

SEQ ID NO: 779 - protein, 119 aa, synthetic construct
EVQLLESGGGLVQPGGSLTLSCAASGRYHMAWFRQAPGKEREFVGTISSGDSTWYTNSVKGRFTISRDNSKNTLYLQMNSLRAEDTAVYYCAAALYYGDSRRAADYPYWGLGTQVTVSS

SEQ ID NO: 780 - protein, 125 aa, synthetic construct
EVQLLESGGGLVQPGGSLTLSCAASGSTFRHHAMAWFRQTPGKEREFVSAINDHGDRTKYLDSVKGRFTISRDNSKNTLYLQMNSLRAEDTAVYYCAAGPLVDYLETTPLVYTYWGLGTQVTVSS

SEQ ID NO: 781 - protein, 124 aa, synthetic construct
EVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSS

SEQ ID NO: 782 - protein, 122 aa, synthetic construct
EVQLLESGGGLVQPGGSLTLSCAASGLTFSSYAMAWFRQAPGKQRELVARITSGGTTDYADSVKGRFTISRDNSKNTLYLQMNSLRAEDTAVYYCAADLTYRNLLLKLPHYWGLGTQVTVSS

SEQ ID NO: 783 - protein, 113 aa, synthetic construct
EVQLLESGGGLVQPGGSLTLSCAASVSIFSVNAVDWYRQSPGKERELVAIMTSDGSTNYDDSVKGRFTISRDNSKNTLYLQMNSLRAEDTAVYYCNTVPPRYWGLGTQVTVSS

SEQ ID NO: 784 - protein, 123 aa, synthetic construct
EVQLLESGGGLVQPGGSLTLSCAASGNDFVITDMHWYRQAPGKQREWVATITRFATTNYADSVKGRFTISRDNSKNTLYLQMNSLRAEDTAVYYCKAIGLRGVPDVNRQFEVWGLGTQVTVSS

SEQ ID NO: 785 - protein, 121 aa, synthetic construct
EVQLLESGGGLVQPGGSLTLSCAASGAIAYIYGMGWYRQAPGKQRELVAAISSGGSTDYADSVKGRFTISRDNSKNTLYLQMNSLRAEDTAVYYCNADVRTSRNLVRSDYWGLGTQVTVSS

SEQ ID NO: 786 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 787 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRSGGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 788 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTGRAAGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 789 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPPASSGRAGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 790 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPIPVQGRAHGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 791 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 792 - protein, 503 aa, synthetic construct; identical to SEQ ID NO: 49

SEQ ID NO: 793 - protein, 513 aa, synthetic construct
EVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSGGGGSGGGSEVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 794 - protein, 249 aa, synthetic construct; identical to SEQ ID NO: 56

SEQ ID NO: 795 - protein, 5 aa, Homo sapiens
APGKG

SEQ ID NO: 796 - protein, 143 aa, synthetic construct; identical to SEQ ID NO: 54

SEQ ID NO: 797 - protein, 143 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRSGGGGGGT

SEQ ID NO: 798 - protein, 143 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTGRAAGGGGGT

SEQ ID NO: 799 - protein, 142 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPPASSGRAGGGGGT

SEQ ID NO: 800 - protein, 142 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPIPVQGRAHGGGGT

SEQ ID NO: 801 - protein, 6 aa, synthetic construct
QDGNEE

SEQ ID NO: 802 - protein, 9 aa, synthetic construct
QDGNEEMGG

SEQ ID NO: 803 - protein, 9 aa, synthetic construct
QDGNEEMGG

SEQ ID NO: 804 - protein, 119 aa, synthetic construct
QVQLQESGGGLVQAGGSLRLSCAASGFIFRAASMAWYRQSPGNERELVASISSGAFTNYADSVKARFTISRDNAKNTVYLQMNSLKPEDTAVYFCGATFLRSDGHHTINGQGTQVTVSS

SEQ ID NO: 805 - protein, 119 aa, synthetic construct
QVQLQESGGGLVQAGGSLRLSCAASGFIFRAASMGWFRQSPGNERELVATVSSGDFTNYADSVKGRFTISRDNAKNTVYLQMNSLKPEDTAVYFCGATFVRSDGHHTIYGQGTQVTVSS

SEQ ID NO: 806 - protein, 119 aa, synthetic construct
QVQLQESGGGLVQAGGSLRLSCAASGFIFRAASMDWYRQFPGNERESIATISSGGFTNYADSVKGRFTISRDNAKNTVYLQMNSLKPEDTAVYFCGATFLRSDGHHTINGQGTQVTVSS

SEQ ID NO: 807 - protein, 119 aa, synthetic construct
QVQLQESGGGLVQAGGSLRLSCAASGFIFRAASMGWFRQSPGNERELVATVSSGGFTNYADSVKGRFTISRDNAKNTVYLQMNSLKPEDTAVYFCGATFVRSDGHHTIYGQGTQVTVSS

SEQ ID NO: 808 - protein, 119 aa, synthetic construct
QVQLQESGGGLVQAGGSLRLSCAASGFIFRAASMDWYRQSPGTQPELVATISSTGFTNYANSVKGRFTISRDNAKNTVYLQMNSLKPEDTAVYFCGATFLRSDGQHSIYGQGTQVTVSS

SEQ ID NO: 809 - protein, 124 aa, synthetic construct
QVQLQESGGGLVHTGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSS

SEQ ID NO: 810 - protein, 124 aa, synthetic construct
QVQLQESGGGLVQAGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSS

SEQ ID NO: 811 - protein, 124 aa, synthetic construct
QVQLQESGGGLVHTGGSLRLSCAFSGDTFLRYAMGWFRQAPGKEREFVAAITWNGGNTDYADSLKGRFTISRDNTKNTVYLQMNSLRPEDTAVYYCAADLTFGLASSHYQYDYWGQGTQVTVSS

SEQ ID NO: 812 - protein, 124 aa, synthetic construct
QVQLQESGGGLVQPGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSS

SEQ ID NO: 813 - protein, 112 aa, synthetic construct
QVQLQESGGGSVLAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSS

SEQ ID NO: 814 - protein, 112 aa, synthetic construct
QVQLQESGGGSVQAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSS

SEQ ID NO: 815 - protein, 112 aa, synthetic construct
QVQLQESGGGLVQPGGSLRLSCAASGFTFSDWAMSWVRQAPGKGLEWVSGIHYGDHTTHYADFVKGRFTISRDDAKNTLYLQMNSLKPEDTAVYYCARGSTKGQGTQVTVSS

SEQ ID NO: 816 - protein, 112 aa, synthetic construct
QVQLQESGGGLVQPGGSLRLSCAASGFTFSDWAMSWVRQAPGKGLEWVSSIHYGDHTTHYADFVKGRFTISRDDAKNTLYLQMNSLKPEDTAVYYCEKGTTRGQGTQVTVSS

SEQ ID NO: 817 - protein, 119 aa, synthetic construct
QVQLQESGGGLVQAGGSLKLSCAASGNVFRAATMAWYRQAPEKQREMVATIASGGTTNYADFVKGRFTISRDNAKNTVYLQMNTLKPEDTAVYYCNAGYLTSLGPKNYWGQGTQVTVSS

SEQ ID NO: 818 - protein, 119 aa, synthetic construct
  VARIANT 44: note = Any amino acid
QVQLQESGGGLVQPGGSLRLSCAASGNVFRAATMAWYRQAPEKXREMVATIASGGTTNYADFVKGRFTISRDNAKNTVYLQMNTLKPEDTAVYYCNAGYLTSLGPKNYWGQGTQVTVSS

SEQ ID NO: 819 - protein, 119 aa, synthetic construct
QVQLQESGGGLVQAGGSLRLSCAASGNVFRAATMAWYRQVPEKQREMVATIASGGTTNYADFVKGRFTISRDNAKNTVYLQMNTLKPEDTAVYYCNALYLTSLGPKSYWGQGTQVTVSS

SEQ ID NO: 820 - protein, 111 aa, synthetic construct
QVQLQESGGGLVQPGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSS

SEQ ID NO: 821 - protein, 111 aa, synthetic construct
QVQLQESGGGLVQAGGSLRLSCAASGFAFGNHWMYWYRQAPGRGRELVASISSGGSTNYVDSVKGRFTISRDNARNTVYLQMYSLRPEDTAVYYCGTSDNWGQGTQVTVSS

SEQ ID NO: 822 - protein, 111 aa, synthetic construct
QVQLQESGGGLVQAGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSS

SEQ ID NO: 823 - protein, 119 aa, synthetic construct
QVQLQESGGGLVQAGGSLRLSCAASGFIFRAASISWYRQSPGNERELVATINSGGFTNYADSVLGRFTISRDNAKNTGYLQMNSLKPEDTAVYFCAATFLRSDGQPPIWGQGTQVTVSS

SEQ ID NO: 824 - protein, 119 aa, synthetic construct
QVQLQESGGGLVQAGGSLRLSCAASGFIFRAASMGWYRQSPGNERELVATINSGGFTNYADSVKGRFTISRDNAKNTGYLQMNSLKPEDTAVYFCAATFLRSDGQPPIWGQGTQVTVSS

SEQ ID NO: 825 - protein, 122 aa, synthetic construct
QVQLQESGGGLVQAGGSLRLSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSS

SEQ ID NO: 826 - protein, 122 aa, synthetic construct
QVQLQESGGGLVQPGASLRVSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSS

SEQ ID NO: 827 - protein, 122 aa, synthetic construct
QVQLQESGGGLVQPGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSS

SEQ ID NO: 828 - protein, 122 aa, synthetic construct
QVQLQESGGGLVQTGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSS

SEQ ID NO: 829 - protein, 119 aa, synthetic construct
QVQLQESGGGLVQAGGSLRLSCAASGSVFRANVMGWYRQAPGKQHELVARIDPGGTTTYADPVKGRFTISRDNAKKTVYLQMNSLKPDDTAVYYCNAIILLSGGPKDYWGQGTQVTVSS

SEQ ID NO: 830 - protein, 120 aa, synthetic construct
QVQLQESGGGLVQAGGSLRLSCAPSGRTSSIFGMGWFRQAPGKEREFVASINWSGGSTSYADSVKGRFTISRDNAKNEMYLQMNSLKFEDTAVYVCAAAVLTNKPSWNFWGQGTQVTVSS

SEQ ID NO: 831 - protein, 121 aa, synthetic construct
QVQLQESGGGLVQAGGSLRLSCAASGPIFSDTIRTMGWYRQAAGKQRELVATIASFPSRTNYVDSVKGRFTISRDIAKNTVYLQMDSLKPEDTAVYYCNVDLASIPTKTYWGQGTQVTVSS

SEQ ID NO: 832 - protein, 120 aa, synthetic construct
QVQLQESGGGLVQAGGSLRLSCAASGSIFGINAMGWYRQAPGKQRESVAFITIGGNTNYLDSVKGRFTISRDNAKNTVYLQMNGLKPEDTAVYYCNTNPPLILTAGGLYWGQGTQVTVSS

SEQ ID NO: 833 - protein, 121 aa, synthetic construct
QVQLQESGGGLVQPGGSLRLSCATSANRFNINVMGWYRQAPGQQRELVATINIGGSTDYADSVKGRFTISRDNAKNTVYLQLSDLKPEDTAVYYCNVKLRVSGPTGPNVYWGQGTQVTVSS

SEQ ID NO: 834 - protein, 116 aa, synthetic construct
QVQLQESGGGLVQAGGSLKLSCTASGTILSTMAWYRQAPGKQRELVATISRGGTTNYSDSVKGRFAISRDSTKNTVYLQMNSLKPEDTAVYYCNTPLTDYGMGYNWGQGTQVTVSS

SEQ ID NO: 835 - protein, 120 aa, synthetic construct
QVQLQESGGGLVQAGGSLRLSCAVSGSIFSLNTLAWYRQAPGRQRDLIARITGGGTTVYADSVKGRFTISRDNAKNTVYLQMNSLKPEDTAVYYCNLMVRHPSGSTYEYWGQGTQVTVSS

SEQ ID NO: 836 - protein, 119 aa, synthetic construct
QVQLQESGGGLVQAGGSLRLSCAASGIIFRGTTMGWFRQAPGKQRESVASISPLGTTSYSGSVEGRFTVSRDNAKNTLFLQMNSLKSEDTAVYYCNAIQVTNVGPRVYWGQGTQVTVSS

SEQ ID NO: 837 - protein, 124 aa, synthetic construct
QVQLQESGGGLVQPGGSLRLSCASSGFTLDDYTIGWFRQAPGKEREGVSCISRRDDSTYYADSVKGRFTISRDNAKNTVDLQMISLRPEDTAVYYCAATPRSYTLRCLGKFDFQGQGTQVTVSS

SEQ ID NO: 838 - protein, 121 aa, synthetic construct
QVQLQESGGGLVQAGGSLRLSCAASGNIVRMTNMAWYRQAPGKQREFVATISAGGSTTYVDSVKDRFTISRDNTKNTVYLQMNYLKPEDTAVYYCATGSILTNRGAIPGSWGHGTQVTVSS

SEQ ID NO: 839 - protein, 127 aa, synthetic construct
QVQLQESGGGLVQAGGSLRLSCAAPGFAFNDHAILWFRQAPGKEREGVSEICRDGTTYYTDSVKGRFTISSDNAKNTVYLQMNSVKTDDTAVYYCAVDRRRYYCSGNRAFSSDYYYWGQGTQVTVSS

SEQ ID NO: 840 - protein, 119 aa, synthetic construct
QVQLQESGGGLVQAGGSLRLSCVHSGSIFRASTMAWYRQAPGKQRELVAQIMSGGGTNYAGSVKGRFTISRDNANNTVYLQMNSLKPEDTAVYYCNAAQITSWGPKVYWGQGTQVTVSS

SEQ ID NO: 841 - protein, 122 aa, synthetic construct
QVQLQESGGGLVQPGGSLRLSCAASGRINSINTMGWYRQAPGNQRELVAEITRGGTTNYADSVQGRYAISRDNAKNLVYLQMNSLKPEDTDVYYCNAQTFPTFSRPTGLDYWGQGTQVTVSS

SEQ ID NO: 842 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGSVFRANVMGWYRQAPGKQHELVARIDPGGTTTYADPVKGRFTISRDNAKKTVYLQMNSLKPDDTAVYYCNAIILLSGGPKDYWGQGTQVTVSSHHHHHH

SEQ ID NO: 843 - protein, 395 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCASSGFTLDDYTIGWFRQAPGKEREGVSCISRRDDSTYYADSVKGRFTISRDNAKNTVDLQMISLRPEDTAVYYCAATPRSYTLRCLGKFDFQGQGTQVTVSSHHHHHH

SEQ ID NO: 844 - protein, 392 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGNIVRMTNMAWYRQAPGKQREFVATISAGGSTTYVDSVKDRFTISRDNTKNTVYLQMNYLKPEDTAVYYCATGSILTNRGAIPGSWGHGTQVTVSSHHHHHH

SEQ ID NO: 845 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASISWYRQSPGNERELVATINSGGFTNYADSVLGRFTISRDNAKNTGYLQMNSLKPEDTAVYFCAATFLRSDGQPPIWGQGTQVTVSSHHHHHH

SEQ ID NO: 846 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGNVFRAATMAWYRQVPEKQREMVATIASGGTTNYADFVKGRFTISRDNAKNTVYLQMNTLKPEDTAVYYCNALYLTSLGPKSYWGQGTQVTVSSHHHHHH

SEQ ID NO: 847 - protein, 398 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAAPGFAFNDHAILWFRQAPGKEREGVSEICRDGTTYYTDSVKGRFTISSDNAKNTVYLQMNSVKTDDTAVYYCAVDRRRYYCSGNRAFSSDYYYWGQGTQVTVSSHHHHHH

SEQ ID NO: 848 - protein, 382 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSSHHHHHH

SEQ ID NO: 849 - protein, 395 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSSHHHHHH

SEQ ID NO: 850 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASMDWYRQFPGNERESIATISSGGFTNYADSVKGRFTISRDNAKNTVYLQMNSLKPEDTAVYFCGATFLRSDGHHTINGQGTQVTVSSHHHHHH

SEQ ID NO: 851 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASMGWFRQSPGNERELVATVSSGGFTNYADSVKGRFTISRDNAKNTVYLQMNSLKPEDTAVYFCGATFVRSDGHHTIYGQGTQVTVSSHHHHHH

SEQ ID NO: 852 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCVHSGSIFRASTMAWYRQAPGKQRELVAQIMSGGGTNYAGSVKGRFTISRDNANNTVYLQMNSLKPEDTAVYYCNAAQITSWGPKVYWGQGTQVTVSSHHHHHH

SEQ ID NO: 853 - protein, 383 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGFTFSDWAMSWVRQAPGKGLEWVSSIHYGDHTTHYADFVKGRFTISRDDAKNTLYLQMNSLKPEDTAVYYCEKGTTRGQGTQVTVSSHHHHHH

SEQ ID NO: 854 - protein, 393 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGASLRVSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSSHHHHHH

SEQ ID NO: 855 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASMDWYRQSPGTQPELVATISSTGFTNYANSVKGRFTISRDNAKNTVYLQMNSLKPEDTAVYFCGATFLRSDGQHSIYGQGTQVTVSSHHHHHH

SEQ ID NO: 856 - protein, 393 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGRINSINTMGWYRQAPGNQRELVAEITRGGTTNYADSVQGRYAISRDNAKNLVYLQMNSLKPEDTDVYYCNAQTFPTFSRPTGLDYWGQGTQVTVSSHHHHHH

SEQ ID NO: 857 - protein, 393 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSSHHHHHH

SEQ ID NO: 858 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLKLSCAASGNVFRAATMAWYRQAPEKQREMVATIASGGTTNYADFVKGRFTISRDNAKNTVYLQMNTLKPEDTAVYYCNAGYLTSLGPKNYWGQGTQVTVSSHHHHHH

SEQ ID NO: 859 - protein, 383 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGSVQAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSSHHHHHH

SEQ ID NO: 860 - protein, 391 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAPSGRTSSIFGMGWFRQAPGKEREFVASINWSGGSTSYADSVKGRFTISRDNAKNEMYLQMNSLKFEDTAVYVCAAAVLTNKPSWNFWGQGTQVTVSSHHHHHH

SEQ ID NO: 861 - protein, 392 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGPIFSDTIRTMGWYRQAAGKQRELVATIASFPSRTNYVDSVKGRFTISRDIAKNTVYLQMDSLKPEDTAVYYCNVDLASIPTKTYWGQGTQVTVSSHHHHHH

SEQ ID NO: 862 - protein, 391 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGSIFGINAMGWYRQAPGKQRESVAFITIGGNTNYLDSVKGRFTISRDNAKNTVYLQMNGLKPEDTAVYYCNTNPPLILTAGGLYWGQGTQVTVSSHHHHHH

SEQ ID NO: 863 - protein, 392 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCATSANRFNINVMGWYRQAPGQQRELVATINIGGSTDYADSVKGRFTISRDNAKNTVYLQLSDLKPEDTAVYYCNVKLRVSGPTGPNVYWGQGTQVTVSSHHHHHH

SEQ ID NO: 864 - protein, 387 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLKLSCTASGTILSTMAWYRQAPGKQRELVATISRGGTTNYSDSVKGRFAISRDSTKNTVYLQMNSLKPEDTAVYYCNTPLTDYGMGYNWGQGTQVTVSSHHHHHH

SEQ ID NO: 865 - protein, 391 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAVSGSIFSLNTLAWYRQAPGRQRDLIARITGGGTTVYADSVKGRFTISRDNAKNTVYLQMNSLKPEDTAVYYCNLMVRHPSGSTYEYWGQGTQVTVSSHHHHHH

SEQ ID NO: 866 - protein, 383 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQPGGSLRLSCAASGFTFSDWAMSWVRQAPGKGLEWVSGIHYGDHTTHYADFVKGRFTISRDDAKNTLYLQMNSLKPEDTAVYYCARGSTKGQGTQVTVSSHHHHHH

SEQ ID NO: 867 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGFIFRAASMAWYRQSPGNERELVASISSGAFTNYADSVKARFTISRDNAKNTVYLQMNSLKPEDTAVYFCGATFLRSDGHHTINGQGTQVTVSSHHHHHH

SEQ ID NO: 868 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSQVQLQESGGGLVQAGGSLRLSCAASGIIFRGTTMGWFRQAPGKQRESVASISPLGTTSYSGSVEGRFTVSRDNAKNTLFLQMNSLKSEDTAVYYCNAIQVTNVGPRVYWGQGTQVTVSSHHHHHH

SEQ ID NO: 869 - protein, 387 aa, synthetic construct
GGGSQVQLQESGGGLVQAGGSLRLSCAASGSVFRANVMGWYRQAPGKQHELVARIDPGGTTTYADPVKGRFTISRDNAKKTVYLQMNSLKPDDTAVYYCNAIILLSGGPKDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 870 - protein, 392 aa, synthetic construct
GGGSQVQLQESGGGLVQPGGSLRLSCASSGFTLDDYTIGWFRQAPGKEREGVSCISRRDDSTYYADSVKGRFTISRDNAKNTVDLQMISLRPEDTAVYYCAATPRSYTLRCLGKFDFQGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 871 - protein, 389 aa, synthetic construct
GGGSQVQLQESGGGLVQAGGSLRLSCAASGNIVRMTNMAWYRQAPGKQREFVATISAGGSTTYVDSVKDRFTISRDNTKNTVYLQMNYLKPEDTAVYYCATGSILTNRGAIPGSWGHGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 872 - protein, 387 aa, synthetic construct
GGGSQVQLQESGGGLVQAGGSLRLSCAASGFIFRAASISWYRQSPGNERELVATINSGGFTNYADSVLGRFTISRDNAKNTGYLQMNSLKPEDTAVYFCAATFLRSDGQPPIWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 873 - protein, 387 aa, synthetic construct
GGGSQVQLQESGGGLVQAGGSLRLSCAASGNVFRAATMAWYRQVPEKQREMVATIASGGTTNYADFVKGRFTISRDNAKNTVYLQMNTLKPEDTAVYYCNALYLTSLGPKSYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 874 - protein, 395 aa, synthetic construct
GGGSQVQLQESGGGLVQAGGSLRLSCAAPGFAFNDHAILWFRQAPGKEREGVSEICRDGTTYYTDSVKGRFTISSDNAKNTVYLQMNSVKTDDTAVYYCAVDRRRYYCSGNRAFSSDYYYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 875 - protein, 379 aa, synthetic construct
GGGSQVQLQESGGGLVQPGGSLRLSCAASGFAFGNHWMYWYRQAPGRGRELVASISSGGSTNYVDSVKGRFTISRDNARNTVYLQMYSLKPEDTAVYYCGTSDNWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 876 - protein, 392 aa, synthetic construct
GGGSQVQLQESGGGLVQPGGSLRLSCAASGDTFLRYAMGWFRQAPGKEREFVAAITWNGGNTDYAGSLKGRFTISRDNTKNTVYLQMNSLKPEDTAVYYCAADLTFGLASSHYQYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 877 - protein, 387 aa, synthetic construct
GGGSQVQLQESGGGLVQAGGSLRLSCAASGFIFRAASMDWYRQFPGNERESIATISSGGFTNYADSVKGRFTISRDNAKNTVYLQMNSLKPEDTAVYFCGATFLRSDGHHTINGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 878 - protein, 387 aa, synthetic construct
GGGSQVQLQESGGGLVQAGGSLRLSCAASGFIFRAASMGWFRQSPGNERELVATVSSGGFTNYADSVKGRFTISRDNAKNTVYLQMNSLKPEDTAVYFCGATFVRSDGHHTIYGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 879 - protein, 387 aa, synthetic construct
GGGSQVQLQESGGGLVQAGGSLRLSCVHSGSIFRASTMAWYRQAPGKQRELVAQIMSGGGTNYAGSVKGRFTISRDNANNTVYLQMNSLKPEDTAVYYCNAAQITSWGPKVYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 880 - protein, 380 aa, synthetic construct
GGGSQVQLQESGGGLVQPGGSLRLSCAASGFTFSDWAMSWVRQAPGKGLEWVSSIHYGDHTTHYADFVKGRFTISRDDAKNTLYLQMNSLKPEDTAVYYCEKGTTRGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 881 - protein, 390 aa, synthetic construct
GGGSQVQLQESGGGLVQPGASLRVSCAASEYILSMYRMAWYRQAPGKVRELVADMSSGGTTNYADFVKGRFTISRDNDRNTVYLQMNRLQPEDTAAYYCNVAGRTGPPSYDAFNNWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 882 - protein, 387 aa, synthetic construct
GGGSQVQLQESGGGLVQAGGSLRLSCAASGFIFRAASMDWYRQSPGTQPELVATISSTGFTNYANSVKGRFTISRDNAKNTVYLQMNSLKPEDTAVYFCGATFLRSDGQHSIYGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 883 - protein, 390 aa, synthetic construct
GGGSQVQLQESGGGLVQPGGSLRLSCAASGRINSINTMGWYRQAPGNQRELVAEITRGGTTNYADSVQGRYAISRDNAKNLVYLQMNSLKPEDTDVYYCNAQTFPTFSRPTGLDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 884 - protein, 390 aa, synthetic construct
GGGSQVQLQESGGGLVQPGGSLRLSCAASESISSFIAVGWYRQAPGKERELVAGINRSGFTYYTDSVKGRFSISRDNAKNTVLLQMTSLKPEDTAVYYCNAGGLYFSNAYTQGDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 885 - protein, 387 aa, synthetic construct
GGGSQVQLQESGGGLVQAGGSLKLSCAASGNVFRAATMAWYRQAPEKQREMVATIASGGTTNYADFVKGRFTISRDNAKNTVYLQMNTLKPEDTAVYYCNAGYLTSLGPKNYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 886 - protein, 380 aa, synthetic construct
GGGSQVQLQESGGGSVQAGGSLRLSCAASGFTFSSYYMSWVRQAPGKGLEWVSGIHYTGDWTNYADSVKGRFTISRDNAKNELYLEMNNLKPEDTAVYYCARGSDKGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 887 - protein, 388 aa, synthetic construct
GGGSQVQLQESGGGLVQAGGSLRLSCAPSGRTSSIFGMGWFRQAPGKEREFVASINWSGGSTSYADSVKGRFTISRDNAKNEMYLQMNSLKFEDTAVYVCAAAVLTNKPSWNFWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 888 - protein, 389 aa, synthetic construct
GGGSQVQLQESGGGLVQAGGSLRLSCAASGPIFSDTIRTMGWYRQAAGKQRELVATIASFPSRTNYVDSVKGRFTISRDIAKNTVYLQMDSLKPEDTAVYYCNVDLASIPTKTYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 889 - protein, 388 aa, synthetic construct
GGGSQVQLQESGGGLVQAGGSLRLSCAASGSIFGINAMGWYRQAPGKQRESVAFITIGGNTNYLDSVKGRFTISRDNAKNTVYLQMNGLKPEDTAVYYCNTNPPLILTAGGLYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 890 - protein, 389 aa, synthetic construct
GGGSQVQLQESGGGLVQPGGSLRLSCATSANRFNINVMGWYRQAPGQQRELVATINIGGSTDYADSVKGRFTISRDNAKNTVYLQLSDLKPEDTAVYYCNVKLRVSGPTGPNVYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 891 - protein, 384 aa, synthetic construct
GGGSQVQLQESGGGLVQAGGSLKLSCTASGTILSTMAWYRQAPGKQRELVATISRGGTTNYSDSVKGRFAISRDSTKNTVYLQMNSLKPEDTAVYYCNTPLTDYGMGYNWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 892 - protein, 388 aa, synthetic construct
GGGSQVQLQESGGGLVQAGGSLRLSCAVSGSIFSLNTLAWYRQAPGRQRDLIARITGGGTTVYADSVKGRFTISRDNAKNTVYLQMNSLKPEDTAVYYCNLMVRHPSGSTYEYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 893 - protein, 380 aa, synthetic construct
GGGSQVQLQESGGGLVQPGGSLRLSCAASGFTFSDWAMSWVRQAPGKGLEWVSGIHYGDHTTHYADFVKGRFTISRDDAKNTLYLQMNSLKPEDTAVYYCARGSTKGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 894 - protein, 387 aa, synthetic construct
GGGSQVQLQESGGGLVQAGGSLRLSCAASGFIFRAASMAWYRQSPGNERELVASISSGAFTNYADSVKARFTISRDNAKNTVYLQMNSLKPEDTAVYFCGATFLRSDGHHTINGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 895 - protein, 387 aa, synthetic construct
GGGSQVQLQESGGGLVQAGGSLRLSCAASGIIFRGTTMGWFRQAPGKQRESVASISPLGTTSYSGSVEGRFTVSRDNAKNTLFLQMNSLKSEDTAVYYCNAIQVTNVGPRVYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLHHHHHH

SEQ ID NO: 896 - protein, 111 aa, synthetic construct
EVQLVESGGGLVQPGGSLTLSCAASGFAFGNHWMYWYRQAPGRGRELVASISSGGSTNYVDSVKGRFTISRDNAKNTLYLQMNSLRAEDTAVYYCGTSDNWGQGTLVTVSS

SEQ ID NO: 897 - protein, 119 aa, synthetic construct
EVQLLESGGGLVQPGGSLTLSCAASGFIFRAASMDWYRQFPGNERESIATISSGGFTNYADSVKGRFTISRDNSKNTLYLQMNSLRAEDTAVYYCGATFLRSDGHHTINGQGTLVTVSS

SEQ ID NO: 898 - protein, 119 aa, synthetic construct
EVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSS

SEQ ID NO: 899 - protein, 382 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLVESGGGLVQPGGSLTLSCAASGFAFGNHWMYWYRQAPGRGRELVASISSGGSTNYVDSVKGRFTISRDNAKNTLYLQMNSLRAEDTAVYYCGTSDNWGQGTLVTVSSHHHHHH

SEQ ID NO: 900 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMDWYRQFPGNERESIATISSGGFTNYADSVKGRFTISRDNSKNTLYLQMNSLRAEDTAVYYCGATFLRSDGHHTINGQGTLVTVSSHHHHHH

SEQ ID NO: 901 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 902 - protein, 12 aa, Homo sapiens
QDGNEEMGGITQ

SEQ ID NO: 903 - protein, 6 aa, synthetic construct
LVQPGN

SEQ ID NO: 904 - protein, 6 aa, synthetic construct
DSVKGR

SEQ ID NO: 905 - protein, 6 aa, synthetic construct
SLRPED

SEQ ID NO: 906 - protein, 507 aa, synthetic construct
EVQLVESGGGLVQPGNSLRLSCAASGFTFSKFGMSWVRQAPGKGLEWVSSISGSGRDTLYADSVKGRFTISRDNAKTTLYLQMNSLRPEDTAVYYCTIGGSLSVSSQGTLVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 907 - protein, 383 aa, synthetic construct
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 908 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 909 - protein, 10 aa, synthetic construct
PQGSTARSAG

SEQ ID NO: 910 - protein, 10 aa, synthetic construct
VANLASTGRA

SEQ ID NO: 911 - protein, 9 aa, synthetic construct
PLGLSTGRA

SEQ ID NO: 912 - protein, 8 aa, synthetic construct
PQASTGRA

SEQ ID NO: 913 - protein, 10 aa, synthetic construct
PKPQASTGRA

SEQ ID NO: 914 - protein, 10 aa, synthetic construct
PTGLESTGRA

SEQ ID NO: 915 - protein, 10 aa, synthetic construct
PENFFSTGRA

SEQ ID NO: 916 - protein, 10 aa, synthetic construct
AATLKSTGRA

SEQ ID NO: 917 - protein, 10 aa, synthetic construct
PQDLLSTGRA

SEQ ID NO: 918 - protein, 9 aa, synthetic construct
PAGLSTGRA

SEQ ID NO: 919 - protein, 9 aa, synthetic construct
VLGLSTGRA

SEQ ID NO: 920 - protein, 9 aa, synthetic construct
VANLASTGA

SEQ ID NO: 921 - protein, 8 aa, synthetic construct
PLGLSTGA

SEQ ID NO: 922 - protein, 7 aa, synthetic construct
PQASTGA

SEQ ID NO: 923 - protein, 9 aa, synthetic construct
PKPQASTGA

SEQ ID NO: 924 - protein, 9 aa, synthetic construct
PTGLESTGA

SEQ ID NO: 925 - protein, 9 aa, synthetic construct
PENFFSTGA

SEQ ID NO: 926 - protein, 9 aa, synthetic construct
AATLKSTGA

SEQ ID NO: 927 - protein, 9 aa, synthetic construct
PQDLLSTGA

SEQ ID NO: 928 - protein, 8 aa, synthetic construct
PAGLSTGA

SEQ ID NO: 929 - protein, 8 aa, synthetic construct
VLGLSTGA

SEQ ID NO: 930 - protein, 9 aa, synthetic construct
RTRYEDGGS

SEQ ID NO: 931 - protein, 9 aa, synthetic construct
PQASTGGSG

SEQ ID NO: 932 - protein, 19 aa, synthetic construct
GGGGPQASTGRSGGGGGGT

SEQ ID NO: 933 - protein, 19 aa, synthetic construct
GGGGPQGSTGRAAGGGGGT

SEQ ID NO: 934 - protein, 19 aa, synthetic construct
GGGGPQGSTARSAGGGGGT

SEQ ID NO: 935 - protein, 18 aa, synthetic construct
GGGGPIPVQGRAHGGGGT

SEQ ID NO: 936 - protein, 19 aa, synthetic construct
GGGGVANLASTGRAGGGGT

SEQ ID NO: 937 - protein, 18 aa, synthetic construct
GGGGPLGLSTGRAGGGGT

SEQ ID NO: 938 - protein, 16 aa, synthetic construct
GGGGPQASTGRAGGGT

SEQ ID NO: 939 - protein, 18 aa, synthetic construct
GGGGPKPQASTGRAGGGT

SEQ ID NO: 940 - protein, 18 aa, synthetic construct
GGGGPTGLESTGRAGGGT

SEQ ID NO: 941 - protein, 18 aa, synthetic construct
GGGGPENFFSTGRAGGGT

SEQ ID NO: 942 - protein, 18 aa, synthetic construct
GGGGAATLKSTGRAGGGT

SEQ ID NO: 943 - protein, 18 aa, synthetic construct
GGGGPQDLLSTGRAGGGT

SEQ ID NO: 944 - protein, 17 aa, synthetic construct
GGGGPAGLSTGRAGGGT

SEQ ID NO: 945 - protein, 17 aa, synthetic construct
GGGGVLGLSTGRAGGGT

SEQ ID NO: 946 - protein, 17 aa, synthetic construct
GGGGVANLASTGAGGGT

SEQ ID NO: 947 - protein, 16 aa, synthetic construct
GGGGPLGLSTGAGGGT

SEQ ID NO: 948 - protein, 15 aa, synthetic construct
GGGGPQASTGAGGGT

SEQ ID NO: 949 - protein, 17 aa, synthetic construct
GGGGPKPQASTGAGGGT

SEQ ID NO: 950 - protein, 17 aa, synthetic construct
GGGGPTGLESTGAGGGT

SEQ ID NO: 951 - protein, 17 aa, synthetic construct
GGGGPENFFSTGAGGGT

SEQ ID NO: 952 - protein, 17 aa, synthetic construct
GGGGAATLKSTGAGGGT

SEQ ID NO: 953 - protein, 17 aa, synthetic construct
GGGGPQDLLSTGAGGGT

SEQ ID NO: 954 - protein, 16 aa, synthetic construct
GGGGPAGLSTGAGGGT

SEQ ID NO: 955 - protein, 16 aa, synthetic construct
GGGGVLGLSTGAGGGT

SEQ ID NO: 956 - protein, 17 aa, synthetic construct
GGGGRTRYEDGGSGGGT

SEQ ID NO: 957 - protein, 17 aa, synthetic construct
GGGGPQASTGGSGGGGT

SEQ ID NO: 958 - protein, 19 aa, synthetic construct
GGGGKPLGLQARVVGGGGS

SEQ ID NO: 959 - protein, 19 aa, synthetic construct
GGGGPQASTGRSGGGGGGS

SEQ ID NO: 960 - protein, 19 aa, synthetic construct
GGGGPQGSTGRAAGGGGGS

SEQ ID NO: 961 - protein, 19 aa, synthetic construct
GGGGPQGSTARSAGGGGGS

SEQ ID NO: 962 - protein, 18 aa, synthetic construct
GGGGPIPVQGRAHGGGGS

SEQ ID NO: 963 - protein, 19 aa, synthetic construct
GGGGVANLASTGRAGGGGS

SEQ ID NO: 964 - protein, 18 aa, synthetic construct
GGGGPLGLSTGRAGGGGS

SEQ ID NO: 965 - protein, 17 aa, synthetic construct
GGGGPQASTGRAGGGTS

SEQ ID NO: 966 - protein, 18 aa, synthetic construct
GGGGPKPQASTGRAGGGS

SEQ ID NO: 967 - protein, 18 aa, synthetic construct
GGGGPTGLESTGRAGGGS

SEQ ID NO: 968 - protein, 18 aa, synthetic construct
GGGGPENFFSTGRAGGGS

SEQ ID NO: 969 - protein, 18 aa, synthetic construct
GGGGAATLKSTGRAGGGS

SEQ ID NO: 970 - protein, 18 aa, synthetic construct
GGGGPQDLLSTGRAGGGS

SEQ ID NO: 971 - protein, 17 aa, synthetic construct
GGGGPAGLSTGRAGGGS

SEQ ID NO: 972 - protein, 17 aa, synthetic construct
GGGGVLGLSTGRAGGGS

SEQ ID NO: 973 - protein, 17 aa, synthetic construct
GGGGVANLASTGAGGGS

SEQ ID NO: 974 - protein, 16 aa, synthetic construct
GGGGPLGLSTGAGGGS

SEQ ID NO: 975 - protein, 15 aa, synthetic construct
GGGGPQASTGAGGGS

SEQ ID NO: 976 - protein, 17 aa, synthetic construct
GGGGPKPQASTGAGGGS

SEQ ID NO: 977 - protein, 17 aa, synthetic construct
GGGGPTGLESTGAGGGS

SEQ ID NO: 978 - protein, 17 aa, synthetic construct
GGGGPENFFSTGAGGGS

SEQ ID NO: 979 - protein, 17 aa, synthetic construct
GGGGAATLKSTGAGGGS

SEQ ID NO: 980 - protein, 17 aa, synthetic construct
GGGGPQDLLSTGAGGGS

SEQ ID NO: 981 - protein, 16 aa, synthetic construct
GGGGPAGLSTGAGGGS

SEQ ID NO: 982 - protein, 16 aa, synthetic construct
GGGGVLGLSTGAGGGS

SEQ ID NO: 983 - protein, 17 aa, synthetic construct
GGGGRTRYEDGGSGGGS

SEQ ID NO: 984 - protein, 17 aa, synthetic construct
GGGGPQASTGGSGGGGS

SEQ ID NO: 985 - protein, 5 aa, synthetic construct
VANLA

SEQ ID NO: 986 - protein, 4 aa, synthetic construct
PLGL

SEQ ID NO: 987 - protein, 4 aa, synthetic construct
PQAS

SEQ ID NO: 988 - protein, 5 aa, synthetic construct
PKPQA

SEQ ID NO: 989 - protein, 5 aa, synthetic construct
PTGLE

SEQ ID NO: 990 - protein, 5 aa, synthetic construct
PENFF

SEQ ID NO: 991 - protein, 5 aa, synthetic construct
AATLK

SEQ ID NO: 992 - protein, 5 aa, synthetic construct
PQDLL

SEQ ID NO: 993 - protein, 4 aa, synthetic construct
PAGL

SEQ ID NO: 994 - protein, 4 aa, synthetic construct
VLGL

SEQ ID NO: 995 - protein, 6 aa, synthetic construct
RTRYED

SEQ ID NO: 996 - protein, 5 aa, synthetic construct
KPLGL

SEQ ID NO: 997 - protein, 119 aa, synthetic construct
EVQLLESGGGLVQPGGSLTLSCAASGFIFRAASMDWYRQFPGNERESIATISSGGFTNYADSVKGRFTISRDNSKNTLYLQMNSLRAEDTAVYYCGATFLRSDGHHTIWGQGTLVTVSS

SEQ ID NO: 998 - protein, 119 aa, synthetic construct
EVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSS

SEQ ID NO: 999 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRSGGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1000 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTGRAAGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1001 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTARSAGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1002 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGGSGGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1003 - protein, 525 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPENFFSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1004 - protein, 525 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGAATLKSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1005 - protein, 525 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQDLLSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1006 - protein, 524 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVLGLSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1007 - protein, 524 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVANLASTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1008 - protein, 523 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVLGLSTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1009 - protein, 525 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGRTRYEDGGSGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1010 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 1011 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGSGGGGSGGVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1012 - protein, 390 aa, synthetic construct
VVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1013 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1014 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRSGGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1015 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTGRAAGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1016 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTARSAGGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1017 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGGPIPVQGRAHGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1018 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVANLASTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1019 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPLGLSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1020 - protein, 529 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1021 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPKPQASTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1022 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPTGLESTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1023 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPENFFSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1024 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGAATLKSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1025 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQDLLSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1026 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPAGLSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1027 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVLGLSTGRAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1028 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVANLASTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1029 - protein, 529 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPLGLSTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1030 - protein, 528 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1031 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPKPQASTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1032 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPTGLESTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1033 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPENFFSTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1034 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGAATLKSTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1035 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQDLLSTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1036 - protein, 529 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPAGLSTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1037 - protein, 529 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVLGLSTGAGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1038 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRSGGGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1039 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTGRAAGGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1040 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTARSAGGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1041 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGGSGGGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1042 - protein, 525 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPENFFSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1043 - protein, 525 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGAATLKSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1044 - protein, 525 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQDLLSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1045 - protein, 524 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVLGLSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1046 - protein, 524 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVANLASTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1047 - protein, 523 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVLGLSTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1048 - protein, 525 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGRTRYEDGGSGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTIWGQGTLVTVSSHHHHHH

SEQ ID NO: 1049 - protein, 526 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 1050 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGKPLGLQARVVGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1051 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRSGGGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1052 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTGRAAGGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1053 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQGSTARSAGGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1054 - protein, 532 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGGPIPVQGRAHGGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1055 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVANLASTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1056 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPLGLSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1057 - protein, 529 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1058 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPKPQASTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1059 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPTGLESTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1060 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPENFFSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1061 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGAATLKSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1062 - protein, 531 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQDLLSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1063 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPAGLSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1064 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVLGLSTGRAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1065 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVANLASTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1066 - protein, 529 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPLGLSTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1067 - protein, 528 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQASTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1068 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPKPQASTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1069 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPTGLESTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1070 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPENFFSTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1071 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGAATLKSTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1072 - protein, 530 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPQDLLSTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1073 - protein, 529 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGPAGLSTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1074 - protein, 529 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGVLGLSTGAGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGEVQLLESGGGLVQPGGSLTLSCAASGRTDSWYVMGWFRQAPGKDREFVAGVSWSYGNTYYADSVKGRFTISRDNSKNTLYLQMNSLRAEDTAVYYCAARVSREVIPTRWDLYNYWGLGTQVTVSSHHHHHH

SEQ ID NO: 1075 - protein, 372 aa, synthetic construct
MALPVTALLLPLALLLHAARPEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1076 - protein, 522 aa, synthetic construct
MALPVTALLLPLALLLHAARPEQKLISEEDLEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGSGGGQAGVVGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1077 - protein, 18 aa, synthetic construct
GGGGPLGLQAGVVGGGGT

SEQ ID NO: 1078 - protein, 522 aa, synthetic construct
MALPVTALLLPLALLLHAARPEQKLISEEDLEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGPLGLQAGVVGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1079 - protein, 18 aa, synthetic construct
GGGGPLNLQAGVVGGGGT

SEQ ID NO: 1080 - protein, 522 aa, synthetic construct
MALPVTALLLPLALLLHAARPEQKLISEEDLEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGPLNLQAGVVGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1081 - protein, 18 aa, synthetic construct
GGGGPAGLQAGVVGGGGT

SEQ ID NO: 1082 - protein, 522 aa, synthetic construct
MALPVTALLLPLALLLHAARPEQKLISEEDLEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGPAGLQAGVVGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1083 - protein, 18 aa, synthetic construct
GGGGPANLQAGVVGGGGT

SEQ ID NO: 1084 - protein, 522 aa, synthetic construct
MALPVTALLLPLALLLHAARPEQKLISEEDLEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGPANLQAGVVGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1085 - protein, 511 aa, synthetic construct
MALPVTALLLPLALLLHAARPEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1086 - protein, 17 aa, synthetic construct
GGGGEYSGLSDGGGGGT

SEQ ID NO: 1087 - protein, 511 aa, synthetic construct
MALPVTALLLPLALLLHAARPEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGEYSGLSDGGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1088 - protein, 17 aa, synthetic construct
GGGGKPAEFTVDGGGGT

SEQ ID NO: 1089 - protein, 17 aa, synthetic construct
GGGGAISELFAKGGGGT

SEQ ID NO: 1090 - protein, 522 aa, synthetic construct
MALPVTALLLPLALLLHAARPEQKLISEEDLEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGSGGGGSGGGGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1091 - protein, 15 aa, synthetic construct
GGGGGDFLAEGGGGT

SEQ ID NO: 1092 - protein, 519 aa, synthetic construct
MALPVTALLLPLALLLHAARPEQKLISEEDLEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGGDFLAEGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1093 - protein, 15 aa, synthetic construct
GGGGAGPLIVGGGGT

SEQ ID NO: 1094 - protein, 519 aa, synthetic construct
MALPVTALLLPLALLLHAARPEQKLISEEDLEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGAGPLIVGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1095 - protein, 15 aa, synthetic construct
GGGGLRELHLGGGGT

SEQ ID NO: 1096 - protein, 519 aa, synthetic construct
MALPVTALLLPLALLLHAARPEQKLISEEDLEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGLRELHLGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1097 - protein, 516 aa, synthetic construct
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGGGGPPASSGRAGGGGGGTEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1098 - protein, 514 aa, synthetic construct
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGGGGLSGRSDNHGGGGTEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFASDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1099 - protein, 516 aa, synthetic construct
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGGGGPPASSGRAGGGGGGTEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFASDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1100 - protein, 514 aa, synthetic construct
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFASDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1101 - protein, 377 aa, synthetic construct
MALPVTALLLPLALLLHAARPEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFASDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1102 - protein, 511 aa, synthetic construct
MALPVTALLLPLALLLHAARPEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGKPAEFTVDGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1103 - protein, 511 aa, synthetic construct
MALPVTALLLPLALLLHAARPEVQLVESGGGLVQPGNSLRLSCAASGFTFSKFGMSWVRQRQSTFMAMDMPILEWVSSISGSGRDTLYADSVKGRFTISRDNAKTTLYLQMNSLRPEDTAVYYCTIGGSLSVSSQGTLVTVSSGGGGAISELFAKGGGGTEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1104 - protein, 253 aa, synthetic construct
MALPVTALLLPLALLLHAARPTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1105 - protein, 377 aa, synthetic construct
MALPVTALLLPLALLLHAARPEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1106 - protein, 514 aa, synthetic construct
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGSGGSGGGGSGGGGGSGEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1107 - protein, 514 aa, synthetic construct
MALPVTALLLPLALLLHAARPEVQLVESGGGLVQPGNSLRLSCAASGFTFSKFGMSWVRQANPWVPPREGLEWVSSISGSGRDTLYADSVKGRFTISRDNAKTTLYLQMNSLRPEDTAVYYCTIGGSLSVSSQGTLVTVSSGGGGLSGRSDNHGGGGTEVQLLESGGGLVQPGGSLTLSCAASGRSFSNYIMGWFRQAPGKEREFVAGLGWSPGNTYYADSVKGRFTISRDNSKNTLYLQMNSLRAEDTAVYYCAARRGDVIYTTPWNYVYWGLGTQVTVSSTSDYKDDDDKTTTPAPRPPTPAPTIASQPLSLRPEACRPAAGGAVHTRGLDFACDIYIWAPLAGTCGVLLLSLVITLYKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1108 - protein, 8 aa, synthetic construct
EYSGLSDG

SEQ ID NO: 1109 - protein, 8 aa, synthetic construct
KPAEFTVD

SEQ ID NO: 1110 - protein, 8 aa, synthetic construct
AISELFAK

SEQ ID NO: 1111 - protein, 6 aa, synthetic construct
GDFLAE

SEQ ID NO: 1112 - protein, 6 aa, synthetic construct
AGPLIV

SEQ ID NO: 1113 - protein, 6 aa, synthetic construct
LRELHL

SEQ ID NO: 1114 - protein, 4 aa, synthetic construct
PLGL

SEQ ID NO: 1115 - protein, 4 aa, synthetic construct
PLNL

SEQ ID NO: 1116 - protein, 4 aa, synthetic construct
PAGL

SEQ ID NO: 1117 - protein, 4 aa, synthetic construct
PANL

SEQ ID NO: 1118 - protein, 119 aa, synthetic construct; identical to SEQ ID NO: 998

SEQ ID NO: 1119 - protein, 10 aa, synthetic construct
PQASTGGSGG

SEQ ID NO: 1120 - protein, 8 aa, synthetic construct
GPLGIAGQ

SEQ ID NO: 1121 - protein, 8 aa, synthetic construct
AVRWLLTA

SEQ ID NO: 1122 - protein, 522 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGGDFLAEGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 1123 - protein, 522 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGAGPLIVGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 1124 - protein, 522 aa, synthetic construct
EVQLVESGGGLVQPGNSLRLSCAASGFTFSKFGMSWVRQGGGGGLDGNEEPGGLEWVSSISGSGRDTLYADSVKGRFTISRDNAKTTLYLQMNSLRPEDTAVYYCTIGGSLSVSSQGTLVTVSSGGGGLRELHLGGGGTQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGSEVQLLESGGGLVQPGGSLTLSCAASGFIFRAASMAWYRQSPGNERELVASISSGAFTNYADSVKARFTISRDNSKNTLYLQMNSLRAEDTAVYYCGATFLRSDGHHTINGQGTLVTVSSHHHHHH

SEQ ID NO: 1125 - protein, 19 aa, synthetic construct
GGGGPQASTGGSGGGGGGT

SEQ ID NO: 1126 - protein, 19 aa, synthetic construct
GGGGPQASTGGSGGGGGGS

SEQ ID NO: 1127 - protein, 15 aa, synthetic construct
GGGGLRELHLGGGGS

SEQ ID NO: 1128 - protein, 15 aa, synthetic construct
GGGGLRELHLGGGGT

SEQ ID NO: 1129 - protein, 114 aa, synthetic construct
QVQLQESGGGLVQAGGSLRLSCAASGSVSMFNSMGWHRQPPGKQRELVAIITSGGSSNYADTVKGRFTISRDNTKNTVYLQMNDLKPEDTAVYYCFYYFQSSYWGQGTQVTVSS

SEQ ID NO: 1130 - protein, 119 aa, synthetic construct
EVQLVESGGGLVQPGGSLTLSCVASGKTSSINAMGWYRRAPGKQRELVAGISSDGSKVYADSVKGRFTISRDNAKNSVYLQMNSLRAEDTAVYYCYYFRTVSGVSMRYWGQGTLVTVSS

SEQ ID NO: 1131 - protein, 119 aa, synthetic construct
EVQLVESGGGLVQPGGSLTLSCVASGSTSSINAVGWYRRAPGKQRELVAGISSDGSKVYRDSVKGRFTISRDNAKNSVYLQMNSLRAEDTAVYYCYYFRTVQGSSMRYWGQGTLVTVSS

SEQ ID NO: 1132 - protein, 6 aa, synthetic construct
RRRRRR

SEQ ID NO: 1133 - protein, 5 aa, synthetic construct
GGGGS

SEQ ID NO: 1134 - protein, 5 aa, synthetic construct
GGGGT

SEQ ID NO: 1135 - protein, 6 aa, synthetic construct
HHHHHH

SEQ ID NO: 1136 - protein, 9 aa, synthetic construct
GGGGSGGGS

SEQ ID NO: 1137 - protein, 18 aa, synthetic construct
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 14: note = Any amino acid
GGGGXXXXXXXXXXGGGG

SEQ ID NO: 1138 - protein, 8 aa, synthetic construct
  VARIANT 4: note = Any amino acid
CFCXLCFR

SEQ ID NO: 1139 - protein, 6 aa, synthetic construct
IVLYEW

SEQ ID NO: 1140 - protein, 6 aa, synthetic construct
CFMCYW

SEQ ID NO: 1141 - protein, 7 aa, synthetic construct
FYLAWAA

SEQ ID NO: 1142 - protein, 8 aa, synthetic construct
GLSNRSGC

SEQ ID NO: 1143 - protein, 8 aa, synthetic construct
  VARIANT 8: note = Any amino acid
FFNSEQFX

SEQ ID NO: 1144 - protein, 8 aa, synthetic construct
VYMFNLVD

SEQ ID NO: 1145 - protein, 8 aa, synthetic construct
FGPYNLVS

SEQ ID NO: 1146 - protein, 7 aa, synthetic construct
  VARIANT 2: note = Any amino acid
GXTDGGT

SEQ ID NO: 1147 - protein, 8 aa, synthetic construct
SICIVSFY

SEQ ID NO: 1148 - protein, 6 aa, synthetic construct
  VARIANT 5: note = Any amino acid
  VARIANT 6: note = Any amino acid
LSRPXX

SEQ ID NO: 1149 - protein, 8 aa, synthetic construct
  VARIANT 7: note = Any amino acid
FALQCPXL

SEQ ID NO: 1150 - protein, 8 aa, synthetic construct
  VARIANT 2: note = Any amino acid
SXMSMSLF

SEQ ID NO: 1151 - protein, 9 aa, synthetic construct
  VARIANT 3: note = Any amino acid
GFXIDVFIC

SEQ ID NO: 1152 - protein, 6 aa, synthetic construct
LFQPGN

SEQ ID NO: 1153 - protein, 8 aa, synthetic construct
  VARIANT 3: note = Any amino acid
HYXRISAM

SEQ ID NO: 1154 - protein, 6 aa, synthetic construct
  VARIANT 4: note = Any amino acid
LVQXGN

SEQ ID NO: 1155 - protein, 8 aa, synthetic construct
MLTGFPFG

SEQ ID NO: 1156 - protein, 5 aa, synthetic construct
STQDM

SEQ ID NO: 1157 - protein, 5 aa, synthetic construct
KQHSN

SEQ ID NO: 1158 - protein, 5 aa, synthetic construct
  VARIANT 1: note = Any amino acid
XAPLV

SEQ ID NO: 1159 - protein, 8 aa, synthetic construct
DMWGANCS

SEQ ID NO: 1160 - protein, 8 aa, synthetic construct
INVGSPAY

SEQ ID NO: 1161 - protein, 8 aa, synthetic construct
CSVGISRQ

SEQ ID NO: 1162 - protein, 8 aa, synthetic construct
LRGGLHRV

SEQ ID NO: 1163 - protein, 6 aa, synthetic construct
QLETPC

SEQ ID NO: 1164 - protein, 7 aa, synthetic construct
KLSTIWM

SEQ ID NO: 1165 - protein, 7 aa, synthetic construct
DSGLTLR

SEQ ID NO: 1166 - protein, 7 aa, synthetic construct
  VARIANT 4: note = Any amino acid
  VARIANT 6: note = Any amino acid
  VARIANT 7: note = Any amino acid
FLIXFXX

SEQ ID NO: 1167 - protein, 5 aa, synthetic construct
TNTAS

SEQ ID NO: 1168 - protein, 7 aa, synthetic construct
CPPAIMP

SEQ ID NO: 1169 - protein, 6 aa, synthetic construct
  VARIANT 5: note = Any amino acid
IVGNXT

SEQ ID NO: 1170 - protein, 7 aa, synthetic construct
  VARIANT 5: note = Any amino acid
NVTLXWV

SEQ ID NO: 1171 - protein, 7 aa, synthetic construct
ISLPVMS

SEQ ID NO: 1172 - protein, 7 aa, synthetic construct
LYEPRAE

SEQ ID NO: 1173 - protein, 8 aa, synthetic construct
  VARIANT 8: note = Any amino acid
YGNNCVLX

SEQ ID NO: 1174 - protein, 5 aa, synthetic construct
  VARIANT 2: note = Any amino acid
AXGKG

SEQ ID NO: 1175 - protein, 5 aa, synthetic construct
  VARIANT 1: note = Any amino acid
XEIWW

SEQ ID NO: 1176 - protein, 6 aa, synthetic construct
SSMIQM

SEQ ID NO: 1177 - protein, 7 aa, synthetic construct
LLLIFKF

SEQ ID NO: 1178 - protein, 9 aa, synthetic construct
FLILALTKP

SEQ ID NO: 1179 - protein, 6 aa, synthetic construct
SKWACG

SEQ ID NO: 1180 - protein, 7 aa, synthetic construct
NYRWISL

SEQ ID NO: 1181 - protein, 9 aa, synthetic construct
SVLFGVAYL

SEQ ID NO: 1182 - protein, 8 aa, synthetic construct
MRRPCVIS

SEQ ID NO: 1183 - protein, 6 aa, synthetic construct
LLWHWD

SEQ ID NO: 1184 - protein, 9 aa, synthetic construct
RGLIDNDYQ

SEQ ID NO: 1185 - protein, 6 aa, synthetic construct
DSVKGL

SEQ ID NO: 1186 - protein, 7 aa, synthetic construct
LINLITE

SEQ ID NO: 1187 - protein, 6 aa, synthetic construct
SDNILY

SEQ ID NO: 1188 - protein, 8 aa, synthetic construct
TPRTPTRP

SEQ ID NO: 1189 - protein, 7 aa, synthetic construct
FLCFDLQ

SEQ ID NO: 1190 - protein, 6 aa, synthetic construct
VARAYD

SEQ ID NO: 1191 - protein, 6 aa, synthetic construct
  VARIANT 4: note = Any amino acid
DSVXGR

SEQ ID NO: 1192 - protein, 6 aa, synthetic construct
  VARIANT 1: note = Any amino acid
XVACGR

SEQ ID NO: 1193 - protein, 6 aa, synthetic construct
SLHPED

SEQ ID NO: 1194 - protein, 7 aa, synthetic construct
  VARIANT 5: note = Any amino acid
  VARIANT 7: note = Any amino acid
LMRIXMX

SEQ ID NO: 1195 - protein, 8 aa, synthetic construct
  VARIANT 5: note = Any amino acid
QLMIXHFI

SEQ ID NO: 1196 - protein, 7 aa, synthetic construct
VPSYRVA

SEQ ID NO: 1197 - protein, 8 aa, synthetic construct
VTEMNAMS

SEQ ID NO: 1198 - protein, 6 aa, synthetic construct
LKRTVY

SEQ ID NO: 1199 - protein, 9 aa, synthetic construct
  VARIANT 4: note = Any amino acid
SPDXRQYSV

SEQ ID NO: 1200 - protein, 6 aa, synthetic construct
SLCPED

SEQ ID NO: 1201 - protein, 6 aa, synthetic construct
  VARIANT 1: note = Any amino acid
  VARIANT 5: note = Any amino acid
XFLMXY

SEQ ID NO: 1202 - protein, 7 aa, synthetic construct
YRNHNRN

SEQ ID NO: 1203 - protein, 6 aa, synthetic construct
  VARIANT 4: note = Any amino acid
SLRXED

SEQ ID NO: 1204 - protein, 7 aa, synthetic construct
  VARIANT 6: note = Any amino acid
NTTARXC

SEQ ID NO: 1205 - protein, 7 aa, synthetic construct
GLINSIA

SEQ ID NO: 1206 - protein, 6 aa, synthetic construct
LIQPGN

SEQ ID NO: 1207 - protein, 7 aa, synthetic construct
VAVLRGE

SEQ ID NO: 1208 - protein, 7 aa, synthetic construct
AAADNGV

SEQ ID NO: 1209 - protein, 6 aa, synthetic construct
  VARIANT 6: note = Any amino acid
PGGEGX

SEQ ID NO: 1210 - protein, 5 aa, synthetic construct
YKEWD

SEQ ID NO: 1211 - protein, 5 aa, synthetic construct
  VARIANT 4: note = Any amino acid
ARRXG

SEQ ID NO: 1212 - protein, 6 aa, synthetic construct
RGSEGG

SEQ ID NO: 1213 - protein, 6 aa, synthetic construct
  VARIANT 1: note = Any amino acid
XLHGGS

SEQ ID NO: 1214 - protein, 5 aa, synthetic construct
LTNTS

SEQ ID NO: 1215 - protein, 7 aa, synthetic construct
KQQSHIM

SEQ ID NO: 1216 - protein, 6 aa, synthetic construct
  VARIANT 3: note = Any amino acid
LPXMKR

SEQ ID NO: 1217 - protein, 5 aa, synthetic construct
VGHPK

SEQ ID NO: 1218 - protein, 5 aa, synthetic construct
TVTTP

SEQ ID NO: 1219 - protein, 6 aa, synthetic construct
SNSLNS

SEQ ID NO: 1220 - protein, 5 aa, synthetic construct
TSSRT

SEQ ID NO: 1221 - protein, 5 aa, synthetic construct
VANLY

SEQ ID NO: 1222 - protein, 5 aa, synthetic construct
SEAKT

SEQ ID NO: 1223 - protein, 5 aa, synthetic construct
APGEG

SEQ ID NO: 1224 - protein, 7 aa, synthetic construct
NMIRYPK

SEQ ID NO: 1225 - protein, 5 aa, synthetic construct
RALNL

SEQ ID NO: 1226 - protein, 5 aa, synthetic construct
TSRNG

SEQ ID NO: 1227 - protein, 5 aa, synthetic construct
TNLSY

SEQ ID NO: 1228 - protein, 7 aa, synthetic construct
VKKSQNS

SEQ ID NO: 1229 - protein, 6 aa, synthetic construct
ADDRTH

SEQ ID NO: 1230 - protein, 5 aa, synthetic construct
  VARIANT 4: note = Any amino acid
APNXV

SEQ ID NO: 1231 - protein, 5 aa, synthetic construct
  VARIANT 1: note = Any amino acid
XSISP

SEQ ID NO: 1232 - protein, 6 aa, synthetic construct
DSDKGR

SEQ ID NO: 1233 - protein, 7 aa, synthetic construct
  VARIANT 4: note = Any amino acid
RPGXGLG

SEQ ID NO: 1234 - protein, 7 aa, synthetic construct
GAYFTGR

SEQ ID NO: 1235 - protein, 6 aa, synthetic construct
DSVRGR

SEQ ID NO: 1236 - protein, 6 aa, synthetic construct
YSVKGR

SEQ ID NO: 1237 - protein, 6 aa, synthetic construct
  VARIANT 1: note = Any amino acid
  VARIANT 5: note = Any amino acid
XSADXG

SEQ ID NO: 1238 - protein, 6 aa, synthetic construct
SLPPQD

SEQ ID NO: 1239 - protein, 8 aa, synthetic construct
  VARIANT 8: note = Any amino acid
FPNKAINX

SEQ ID NO: 1240 - protein, 6 aa, synthetic construct
  VARIANT 2: note = Any amino acid
SXNPQD

SEQ ID NO: 1241 - protein, 6 aa, synthetic construct
SLPPED

SEQ ID NO: 1242 - protein, 4 aa, synthetic construct
PQGS

SEQ ID NO: 1243 - protein, 5 aa, synthetic construct
AATKL